SEQUENCE LISTING (193 sequences)

SEQ ID NO: 1 - protein, 355 aa, Homo sapiens
  source 1..355: note = Human CCR8 protein
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 2 - protein, 34 aa, synthetic construct
  source 1..34: note = Sulfated peptide derived from human CCR8
  MOD_RES 17: note = sulfated tyrosine
MDYTLDLSVTTVTDYYYPDIFSSPCDAELIQTNG

SEQ ID NO: 3 - protein, 34 aa, synthetic construct; identical to SEQ ID NO: 2
  source 1..34: note = sulfated peptide derived from human CCR8
  MOD_RES 15: note = sulfated tyrosine
  MOD_RES 17: note = sulfated tyrosine

SEQ ID NO: 4 - protein, 34 aa, synthetic construct; identical to SEQ ID NO: 2
  source 1..34: note = Sulfated peptide derived from human CCR8
  MOD_RES 16..17: note = sulfated tyrosine

SEQ ID NO: 5 - protein, 34 aa, synthetic construct; identical to SEQ ID NO: 2
  source 1..34: note = Sulfated peptide derived from human CCR8
  MOD_RES 15..17: note = sulfated tyrosine

SEQ ID NO: 6 - protein, 5 aa, synthetic construct
  source 1..5: note = CDR sequence of anti-CCR8 antibody
PYAMN

SEQ ID NO: 7 - protein, 19 aa, synthetic construct
  source 1..19: note = CDR sequence of anti-CCR8 antibody
RIRSKSNNYATYYADSVKD

SEQ ID NO: 8 - protein, 17 aa, synthetic construct
  source 1..17: note = CDR sequence of anti-CCR8 antibody
GREAYYRYDGDYYAMDY

SEQ ID NO: 9 - protein, 16 aa, synthetic construct
  source 1..16: note = CDR sequence of anti-CCR8 antibody
RSSKSLLHSNGNTYLY

SEQ ID NO: 10 - protein, 7 aa, synthetic construct
  source 1..7: note = CDR sequence of anti-CCR8 antibody
RMSNLAS

SEQ ID NO: 11 - protein, 9 aa, synthetic construct
  source 1..9: note = CDR sequence of anti-CCR8 antibody
MQHLEYPFT

SEQ ID NO: 12 - protein, 5 aa, synthetic construct
  source 1..5: note = CDR sequence of anti-CCR8 antibody
TYAMN

SEQ ID NO: 13 - protein, 17 aa, synthetic construct
  source 1..17: note = CDR sequence of anti-CCR8 antibody
GREAYYRYDGGYYAMDY

SEQ ID NO: 14 - protein, 5 aa, synthetic construct
  source 1..5: note = CDR sequence of anti-CCR8 antibody
EYTIH

SEQ ID NO: 15 - protein, 16 aa, synthetic construct
  source 1..16: note = CDR sequence of anti-CCR8 antibody
GINPNGDTRYDQKFKG

SEQ ID NO: 16 - protein, 14 aa, synthetic construct
  source 1..14: note = CDR sequence of anti-CCR8 antibody
VARFYGISPYAMDY

SEQ ID NO: 17 - protein, 16 aa, synthetic construct
  source 1..16: note = CDR sequence of anti-CCR8 antibody
RCTQSLLHSNGDTYLH

SEQ ID NO: 18 - protein, 7 aa, synthetic construct
  source 1..7: note = CDR sequence of anti-CCR8 antibody
KVSNRFS

SEQ ID NO: 19 - protein, 9 aa, synthetic construct
  source 1..9: note = CDR sequence of anti-CCR8 antibody
SQSTHVPYT

SEQ ID NO: 20 - protein, 5 aa, synthetic construct
  source 1..5: note = CDR sequence of anti-CCR8 antibody
AYAMN

SEQ ID NO: 21 - protein, 19 aa, synthetic construct
  source 1..19: note = CDR sequence of anti-CCR8 antibody
RIRSKSNDYATYYGDSVKD

SEQ ID NO: 22 - protein, 16 aa, synthetic construct
  source 1..16: note = CDR sequence of anti-CCR8 antibody
TSSKSLLHSNGNTYLY

SEQ ID NO: 23 - protein, 9 aa, synthetic construct
  source 1..9: note = CDR sequence of anti-CCR8 antibody
MQHREYPFT

SEQ ID NO: 24 - protein, 5 aa, synthetic construct
  source 1..5: note = CDR sequence of anti-CCR8 antibody
DYVVT

SEQ ID NO: 25 - protein, 16 aa, synthetic construct
  source 1..16: note = CDR sequence of anti-CCR8 antibody
VIWGGGNTYYNSDLKS

SEQ ID NO: 26 - protein, 9 aa, synthetic construct
  source 1..9: note = CDR sequence of anti-CCR8 antibody
RHRDYALDY

SEQ ID NO: 27 - protein, 16 aa, synthetic construct
  source 1..16: note = CDR sequence of anti-CCR8 antibody
RSSQSLVYSNGNTYLH

SEQ ID NO: 28 - protein, 9 aa, synthetic construct
  source 1..9: note = CDR sequence of anti-CCR8 antibody
SQSTYVPPT

SEQ ID NO: 29 - protein, 5 aa, synthetic construct
  source 1..5: note = CDR sequence of anti-CCR8 antibody
DYTLH

SEQ ID NO: 30 - protein, 17 aa, synthetic construct
  source 1..17: note = CDR sequence of anti-CCR8 antibody
GITPKNGDTRYDPRFKD

SEQ ID NO: 31 - protein, 14 aa, synthetic construct
  source 1..14: note = CDR sequence of anti-CCR8 antibody
VARFYGVSPYAMDY

SEQ ID NO: 32 - protein, 9 aa, synthetic construct
  source 1..9: note = CDR sequence of anti-CCR8 antibody
SQTTHVPYT

SEQ ID NO: 33 - protein, 5 aa, synthetic construct
  source 1..5: note = CDR sequence of anti-CCR8 antibody
NYRMH

SEQ ID NO: 34 - protein, 19 aa, synthetic construct
  source 1..19: note = CDR sequence of anti-CCR8 antibody
VIKVKSDNYGANYAESVKG

SEQ ID NO: 35 - protein, 9 aa, synthetic construct
  source 1..9: note = CDR sequence of anti-CCR8 antibody
PDGFSPFVY

SEQ ID NO: 36 - protein, 11 aa, synthetic construct
  source 1..11: note = CDR sequence of anti-CCR8 antibody
KASQDINSYLS

SEQ ID NO: 37 - protein, 7 aa, synthetic construct
  source 1..7: note = CDR sequence of anti-CCR8 antibody
RANRLVD

SEQ ID NO: 38 - protein, 9 aa, synthetic construct
  source 1..9: note = CDR sequence of anti-CCR8 antibody
LQYGEFPPT

SEQ ID NO: 39 - protein, 19 aa, synthetic construct
  source 1..19: note = CDR sequence of anti-CCR8 antibody
RIRSKSNNYATYYGDSVKD

SEQ ID NO: 40 - protein, 17 aa, synthetic construct
  source 1..17: note = CDR sequence of anti-CCR8 antibody
KSSQSLLNSSNQKNYLA

SEQ ID NO: 41 - protein, 7 aa, synthetic construct
  source 1..7: note = CDR sequence of anti-CCR8 antibody
FASTRES

SEQ ID NO: 42 - protein, 9 aa, synthetic construct
  source 1..9: note = CDR sequence of anti-CCR8 antibody
QQHYSTPYT

SEQ ID NO: 43 - protein, 17 aa, synthetic construct
  source 1..17: note = CDR sequence of anti-CCR8 antibody
GINPNNGNTRYDQKFKG

SEQ ID NO: 44 - protein, 14 aa, synthetic construct
  source 1..14: note = CDR sequence of anti-CCR8 antibody
VARSSGSGPYAMDY

SEQ ID NO: 45 - protein, 16 aa, synthetic construct
  source 1..16: note = CDR sequence of anti-CCR8 antibody
RCTQSLLHTNGDTYLH

SEQ ID NO: 46 - protein, 5 aa, synthetic construct
  source 1..5: note = CDR sequence of anti-CCR8 antibody
GYNMH

SEQ ID NO: 47 - protein, 17 aa, synthetic construct
  source 1..17: note = CDR sequence of anti-CCR8 antibody
AISPGKGDTSYNLKFKG

SEQ ID NO: 48 - protein, 8 aa, synthetic construct
  source 1..8: note = CDR sequence of anti-CCR8 antibody
SGGTPFAY

SEQ ID NO: 49 - protein, 16 aa, synthetic construct
  source 1..16: note = CDR sequence of anti-CCR8 antibody
RSSQSLVHSNGDTYLY

SEQ ID NO: 50 - protein, 5 aa, synthetic construct
  source 1..5: note = CDR sequence of anti-CCR8 antibody
ANAMN

SEQ ID NO: 51 - protein, 12 aa, synthetic construct
  source 1..12: note = CDR sequence of anti-CCR8 antibody
GSDNYIFYAMDY

SEQ ID NO: 52 - protein, 5 aa, synthetic construct
  source 1..5: note = CDR sequence of anti-CCR8 antibody
TNAMN

SEQ ID NO: 53 - protein, 19 aa, synthetic construct
  source 1..19: note = CDR sequence of anti-CCR8 antibody
RIRSKSNYYATYYADSVKD

SEQ ID NO: 54 - protein, 12 aa, synthetic construct
  source 1..12: note = CDR sequence of anti-CCR8 antibody
GREMGNYYSMDY

SEQ ID NO: 55 - protein, 11 aa, synthetic construct
  source 1..11: note = CDR sequence of anti-CCR8 antibody
PTYPGSSGFAY

SEQ ID NO: 56 - protein, 11 aa, synthetic construct
  source 1..11: note = CDR sequence of anti-CCR8 antibody
RASQDISNYLN

SEQ ID NO: 57 - protein, 7 aa, synthetic construct
  source 1..7: note = CDR sequence of anti-CCR8 antibody
YTSRLHS

SEQ ID NO: 58 - protein, 9 aa, synthetic construct
  source 1..9: note = CDR sequence of anti-CCR8 antibody
QQGNKFPPT

SEQ ID NO: 59 - protein, 5 aa, synthetic construct
  source 1..5: note = CDR sequence of anti-CCR8 antibody
EYTMH

SEQ ID NO: 60 - protein, 17 aa, synthetic construct
  source 1..17: note = CDR sequence of anti-CCR8 antibody
GINPNNGDTNYNQKFMG

SEQ ID NO: 61 - protein, 10 aa, synthetic construct
  source 1..10: note = CDR of anti-CCR8 antibody
RLLRRGAMDY

SEQ ID NO: 62 - protein, 15 aa, synthetic construct
  source 1..15: note = CDR sequence of anti-CCR8 antibody
RASENVEYYGTSLMQ

SEQ ID NO: 63 - protein, 7 aa, synthetic construct
  source 1..7: note = CDR sequence of anti-CCR8 antibody
AASNVDS

SEQ ID NO: 64 - protein, 9 aa, synthetic construct
  source 1..9: note = CDR sequence of anti-CCR8 antibody
QQSRKVPWT

SEQ ID NO: 65 - protein, 5 aa, synthetic construct
  source 1..5: note = CDR sequence of anti-CCR8 antibody
GSYMH

SEQ ID NO: 66 - protein, 17 aa, synthetic construct
  source 1..17: note = CDR sequence of anti-CCR8 antibody
RINPYNGATSYNQNFKD

SEQ ID NO: 67 - protein, 8 aa, synthetic construct
  source 1..8: note = CDR sequence of anti-CCR8 antibody
TLLRLLDY

SEQ ID NO: 68 - protein, 16 aa, synthetic construct
  source 1..16: note = CDR sequence of anti-CCR8 antibody
RSSKSLLHSNGITYLY

SEQ ID NO: 69 - protein, 7 aa, synthetic construct
  source 1..7: note = CDR sequence of anti-CCR8 antibody
QMSNLAS

SEQ ID NO: 70 - protein, 9 aa, synthetic construct
  source 1..9: note = CDR sequence of anti-CCR8 antibody
AQNLELPWT

SEQ ID NO: 71 - protein, 5 aa, synthetic construct
  source 1..5: note = CDR sequence of anti-CCR8 antibody
SYWMH

SEQ ID NO: 72 - protein, 17 aa, synthetic construct
  source 1..17: note = CDR sequence of anti-CCR8 antibody
NIWPGSASTNYDEKFKN

SEQ ID NO: 73 - protein, 8 aa, synthetic construct
  source 1..8: note = CDR sequence of anti-CCR8 antibody
GGKGAMDY

SEQ ID NO: 74 - protein, 16 aa, synthetic construct
  source 1..16: note = CDR sequence of anti-CCR8 antibody
RSSQSLENSYGNTYLN

SEQ ID NO: 75 - protein, 7 aa, synthetic construct
  source 1..7: note = CDR sequence of anti-CCR8 antibody
RVSNRFS

SEQ ID NO: 76 - protein, 9 aa, synthetic construct
  source 1..9: note = CDR sequence of anti-CCR8 antibody
LQVTHVPPT

SEQ ID NO: 77 - protein, 5 aa, synthetic construct
  source 1..5: note = CDR sequence of anti-CCR8 antibody
TYAMH

SEQ ID NO: 78 - protein, 17 aa, synthetic construct
  source 1..17: note = CDR sequence of anti-CCR8 antibody
HINPSSGYSNYNQKFKD

SEQ ID NO: 79 - protein, 10 aa, synthetic construct
  source 1..10: note = CDR sequence of anti-CCR8 antibody
SEVRRGYFDV

SEQ ID NO: 80 - protein, 11 aa, synthetic construct
  source 1..11: note = CDR sequence of anti-CCR8 antibody
RTSQDIRNYLN

SEQ ID NO: 81 - protein, 9 aa, synthetic construct
  source 1..9: note = CDR sequence of anti-CCR8 antibody
QQGNTLPPT

SEQ ID NO: 82 - protein, 147 aa, synthetic construct
  source 1..147: note = Variable domain sequence of anti-CCR8 antibody
MLLGLKWVFFVVFYQGVLCEVQLLESGGGLVQPKGSLKLSCAASGFTFNPYAMNWVRQAPGRGLEWVARIRSKSNNYATYYADSVKDRFTISRDDSQDMLYLQMNNLKTEDTAMYYCVRGREAYYRYDGDYYAMDYWGQGTSVTVSS

SEQ ID NO: 83 - protein, 132 aa, synthetic construct
  source 1..132: note = Variable domain sequence of anti-CCR8 antibody
MRCLAEFLGLLVLWIPGAIGDIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 84 - protein, 147 aa, synthetic construct
  source 1..147: note = Variable domain sequence of anti-CCR8 antibody
MLLGLKWVFFVVFYQGVHCEVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRGREAYYRYDGGYYAMDYWGQGTSVTVSS

SEQ ID NO: 85 - protein, 141 aa, synthetic construct
  source 1..141: note = Variable domain sequence of anti-CCR8 antibody
MGWSWIFLFLLSGTAGVLSKVQLQQSGPELVKPGASVKISCKTSGYTFTEYTIHWVQQIHGKSPEWIGGINPNGDTRYDQKFKGKATLTIDKSSSTAYMELRSLTSEDSAVYYCARVARFYGISPYAMDYWGQGTSVTVSS

SEQ ID NO: 86 - protein, 131 aa, synthetic construct
  source 1..131: note = Variable domain sequence of anti-CCR8 antibody
MKLPVRLLVLMFWIPASSSDVVMTQTPLSLPVSLGDQASISCRCTQSLLHSNGDTYLHWYLQKPGQSPNLLIYKVSNRFSGVPDRFSGSGSGTDFTLKINRVEAEDLGVYFCSQSTHVPYTFGGGTNLEIK

SEQ ID NO: 87 - protein, 147 aa, synthetic construct
  source 1..147: note = Variable domain sequence of anti-CCR8 antibody
MLLGLKWVFFVVFYQGVHCEVQLVESGGGLVQPTGSLRLSCAASGFTFNAYAMNWVRQAPGKGLEWVARIRSKSNDYATYYGDSVKDRFTISRDDSQSMLYLQMNNLKTEDTAMYYCVRGREAYYRYDGGYYAMDYWGQGTSVTVSS

SEQ ID NO: 88 - protein, 132 aa, synthetic construct
  source 1..132: note = Variable domain sequence of anti-CCR8 antibody
MRCLAEFLGLLVLWIPGAIGDIVMTQAAPSVSVTPGESVSISCTSSKSLLHSNGNTYLYWFLQRPGQSPQLLIYRMSNLASGVPDRFSGSGSGTAFTLRISRVEAEDVGVYYCMQHREYPFTFGSGTKLEIK

SEQ ID NO: 89 - protein, 136 aa, synthetic construct
  source 1..136: note = Variable domain sequence of anti-CCR8 antibody
MAVLGLLLCLVTFPSCVLSQVQLKESGPGLVAPSQSLSITCTVSGLSMNDYVVTWIRQPPGKGLEWLGVIWGGGNTYYNSDLKSRLSITKDNSKSQVFFKMSSLQTDDTAVYYCARRHRDYALDYWGQGISVTVSS

SEQ ID NO: 90 - protein, 131 aa, synthetic construct
  source 1..131: note = Variable domain sequence of anti-CCR8 antibody
MKLPVRLLVLMFWIPASSSDVVMTQTPLSLPVSLGDQASISCRSSQSLVYSNGNTYLHWYLQKPGQSPKLLIYKVSNRFSGVPDRFSGSGSGTDFTLKISRVEAEDLGVYFCSQSTYVPPTFGGGTKLEIK

SEQ ID NO: 91 - protein, 142 aa, synthetic construct
  source 1..142: note = Variable domain sequence of anti-CCR8 antibody
MGWSWIFLFLLSGTAGVLSEVQLQQSGPELLKPGTSVKISCTTSGYTFSDYTLHWVKQSHGKSLEWIGGITPKNGDTRYDPRFKDKATLTIDKSSSAAYMELRSLTSEDSAVYYCARVARFYGVSPYAMDYWGQGASVTVSS

SEQ ID NO: 92 - protein, 131 aa, synthetic construct
  source 1..131: note = Variable domain sequence of anti-CCR8 antibody
MKLPVRLLVLMFWIPVSNSDVVMTQTPLSLPVSLGDQASISCRCTQSLLHSNGDTYLHWYLQKPGQSPKLLIYKVSNRFSGVPDRFSGSGSGTDFTLEITRVEAEDLGVYFCSQTTHVPYTFGGGTKLEIK

SEQ ID NO: 93 - protein, 139 aa, synthetic construct
  source 1..139: note = Variable domain sequence of anti-CCR8 antibody
MELGLSWVFLVALLNGVQCQVHLVETGGGLVRPGNSLKLSCVTSGFTLSNYRMHWLRQPPGKRLEWIAVIKVKSDNYGANYAESVKGRFTISRDDAKSSVYLQMNRLREEDTATYYCSRPDGFSPFVYWGQGTLVTVSA

SEQ ID NO: 94 - protein, 129 aa, synthetic construct
  source 1..129: note = Variable domain sequence of anti-CCR8 antibody
MDMRTPAQFLGILLLWFPGIKCDIKMTQSPSSMYASLGERVTITCKASQDINSYLSWFQQKPGKSPKTLIYRANRLVDGVPSRFSGSGSGQDYFLTISSLEYEDMGIYYCLQYGEFPPTFGAGTKLELK

SEQ ID NO: 95 - protein, 147 aa, synthetic construct
  source 1..147: note = Variable domain sequence of anti-CCR8 antibody
MLLGLKWVFFVVFYQGVHCEVQLVESGGGLVQPKGSLRLSCAASGFTFNAYAMNWVRQAPGKGLEWVARIRSKSNNYATYYGDSVKDRFTISRDDSQTMLYLQMNNLKTEDTAMYYCVRGREAYYRYDGDYYAMDYWGQGTSVTVSS

SEQ ID NO: 96 - protein, 133 aa, synthetic construct
  source 1..133: note = Variable domain sequence of anti-CCR8 antibody
MESQTQVLMFLLLWVSGACADIVMTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPYTFGGGTKLEIK

SEQ ID NO: 97 - protein, 147 aa, synthetic construct
  source 1..147: note = Variable domain sequence of anti-CCR8 antibody
MLLGLKWVFFVVFYQGVHCEVQLVESGGGLVQPKGSLKLSCAASGFTFNAYAMNWVRQAPGKGLEWLARIRSKSNNYATYYADSVKDRFTISRDDSQSMFYLQMNNLKSEDTAMYYCVRGREAYYRYDGGYYAMDYWGQGTSVTVSS

SEQ ID NO: 98 - protein, 132 aa, synthetic construct
  source 1..132: note = Variable domain sequence of anti-CCR8 antibody
MRCLAEFLGLLVLWIPGAIGEIVMTQAALSAPVTPGESVSMSCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 99 - protein, 142 aa, synthetic construct
  source 1..142: note = Variable domain sequence of anti-CCR8 antibody
MGWSWIFLFLLSGTAGVLSEVQLQQSGPELVKPGASVKISCKTSGYTFTEYTIHWVKQSHGESLEWVGGINPNNGNTRYDQKFKGKATLTIDKSSSPAYMELRSLTSEDSAVYYCARVARSSGSGPYAMDYWGQGTSVTVSS

SEQ ID NO: 100 - protein, 131 aa, synthetic construct
  source 1..131: note = Variable domain sequence of anti-CCR8 antibody
MKLPVRLLVLMFWIPVSSSDVVMTQTPLSLPVSLGDQASISCRCTQSLLHTNGDTYLHWYLQKPGQSPKLLIYKVSNRFSGVPDRFSGSGSGTDFTLKISRVEAEDLGVYFCSQSTHVPYTFGGGTKLEIK

SEQ ID NO: 101 - protein, 136 aa, synthetic construct
  source 1..136: note = Variable domain sequence of anti-CCR8 antibody
MGWSCIILFLVATATDVHSQVQLQQPGAELVKPGASVKMSCKATGYTFTGYNMHWVKQTPGQGLEWIGAISPGKGDTSYNLKFKGKATLTTDKSSSTAYMQLSSLTSADSAVYYCARSGGTPFAYWGQGTLVTVSA

SEQ ID NO: 102 - protein, 131 aa, synthetic construct
  source 1..131: note = Variable domain sequence of anti-CCR8 antibody
MKLPVRLLVLMFWIPASSSDVVMTQTPLSLPVSLGDQASISCRSSQSLVHSNGDTYLYWYLQKPGQSPKLLIYKVSNRFSGVPDRFSGSGSGTDFTLKINRVEAEDLGVYFCSQSTHVPYTFGGGTKLEIK

SEQ ID NO: 103 - protein, 142 aa, synthetic construct
  source 1..142: note = Variable domain sequence of anti-CCR8 antibody
MLLGLKWVFFVVFYQGVHCEVQLVETGGGLVQPKGSLKLSCAASGFTFNANAMNWVRQAPGKGLEWVARIRSKSNNYATYYADSVKDRFTISRDDSQSMLYLQMNNLKTEDTAMYYCVRGSDNYIFYAMDYWGQGTSVTVSS

SEQ ID NO: 104 - protein, 132 aa, synthetic construct
  source 1..132: note = Variable domain sequence of anti-CCR8 antibody
MRCLAEFLGLLVLWIPGAIGDIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGGGTKLEIK

SEQ ID NO: 105 - protein, 142 aa, synthetic construct
  source 1..142: note = Variable domain sequence of anti-CCR8 antibody
MLLGLKWVFFVVFYQGVHCEVQLVETGGGLVQPKGSLKLSCAASGFTFNTNAMNWVRQAPGKGLEWVARIRSKSNYYATYYADSVKDRFTISRDDSQSMLYLQMNNLKTEDTAMYYCVRGREMGNYYSMDYWGQGTSVTVSS

SEQ ID NO: 106 - protein, 141 aa, synthetic construct
  source 1..141: note = Variable domain sequence of anti-CCR8 antibody
MELGLSWVFLVALLNGVQCQVQLVETGGGLVRPGNSLKLSCVTSGFTFSNYRMHWLRQPPGKRLEWIAVIKVKSDNYGANYAESVKGRFTISRDDSKSSVYLQVNRLREEDTATYYCSSPTYPGSSGFAYWGQGTLVTVSA

SEQ ID NO: 107 - protein, 127 aa, synthetic construct
  source 1..127: note = Variable domain sequence of anti-CCR8 antibody
MMSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEQEDIATYFCQQGNKFPPTFGGGTKLEIN

SEQ ID NO: 108 - protein, 138 aa, synthetic construct
  source 1..138: note = Variable domain sequence of anti-CCR8 antibody
MGWSWIFLFLLSGTAGVLSEVQLQQSGPELVKPGASVKISCKTSGYTFTEYTMHWVKQSHGKSLEWIGGINPNNGDTNYNQKFMGKATLTVDKSSSTAYMELRSLTSEDSAVYYCVRRLLRRGAMDYWGQGTSVTVSS

SEQ ID NO: 109 - protein, 131 aa, synthetic construct
  source 1..131: note = Variable domain sequence of anti-CCR8 antibody
MESDTLLLWVLLLWVPGSTGDIVLTQSPASLAVSLGQRATISCRASENVEYYGTSLMQWFQQKPGQPPKLLIYAASNVDSGVPARFSGSGSGTDFSLNIHPVEEDDIAMYFCQQSRKVPWTFGGGTKLDIK

SEQ ID NO: 110 - protein, 136 aa, synthetic construct
  source 1..136: note = Variable domain sequence of anti-CCR8 antibody
MGWSWIFLFLLSGTAGVLSEVQLQQSGPELVKPGASVKISCKASVYSFTGSYMHWVKQSHVKSLEWIGRINPYNGATSYNQNFKDKASLTVDKSSSTAYMELHSLTSEDSAVYYCATTLLRLLDYWGQGTTLTVSS

SEQ ID NO: 111 - protein, 132 aa, synthetic construct
  source 1..132: note = Variable domain sequence of anti-CCR8 antibody
MRFSAQLLGLLVLWIPGSTADIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 112 - protein, 136 aa, synthetic construct
  source 1..136: note = Variable domain sequence of anti-CCR8 antibody
MGWSSIILFLVATASGVHSQVQLQQPGSELVRPGASVKLSCKASGYTFTSYWMHWVKQRPGQGLEWIGNIWPGSASTNYDEKFKNKATLTVDTSSSTAYMQLSSLTSEDSAVYYCIRGGKGAMDYWSQGTSVTVSS

SEQ ID NO: 113 - protein, 131 aa, synthetic construct
  source 1..131: note = Variable domain sequence of anti-CCR8 antibody
MKLPVRLLVLMFWIPVSSSDVVMTQIPLSLPVSLGDQASISCRSSQSLENSYGNTYLNWYLQKPGQSPQLLIYRVSNRFSGVLDRFSGSGSGTDFTLKISRVEAEDLGVYFCLQVTHVPPTFGAGTKLELK

SEQ ID NO: 114 - protein, 138 aa, synthetic construct
  source 1..138: note = Variable domain sequence of anti-CCR8 antibody
MERHWIFLLLLSVTAGVHSQVQVQQSGAELARPGASVKMSCKASGYTFTTYAMHWVKQRPGQGLEWIGHINPSSGYSNYNQKFKDKATLTADKSSSTAYMQLSSLTSEDSAVYYCARSEVRRGYFDVWGAGTTVTVSS

SEQ ID NO: 115 - protein, 127 aa, synthetic construct
  source 1..127: note = Variable domain sequence of anti-CCR8 antibody
MMSSAQFLGLLLLCFQGTRCDIQMTQTTSSLSASLGDRVTISCRTSQDIRNYLNWYQQKPDGTVKLLISYTSRLHSGVPSRFSGSGSGTDYSLTISNLEQEDFATYFCQQGNTLPPTFGGGTRVEIK

SEQ ID NO: 116 - protein, 34 aa, synthetic construct; identical to SEQ ID NO: 2
  source 1..34: note = Peptide derived from the N-terminal extracellular domain of human CCR8

SEQ ID NO: 117 - protein, 34 aa, synthetic construct; identical to SEQ ID NO: 2
  source 1..34: note = Sulfated peptide derived from human CCR8
  MOD_RES 15: note = sulfated tyrosine

SEQ ID NO: 118 - protein, 34 aa, synthetic construct; identical to SEQ ID NO: 2
  source 1..34: note = Sulfated peptide derived from human CCR8
  MOD_RES 16: note = sulfated tyrosine

SEQ ID NO: 119 - protein, 34 aa, synthetic construct; identical to SEQ ID NO: 2
  source 1..34: note = Sulfated peptide derived from human CCR8
  MOD_RES 15..16: note = sulfated tyrosine

SEQ ID NO: 120 - protein, 122 aa, synthetic construct
  source 1..122: note = Human CCR8 specific benchmark antibody Ref#1 - VH region
EVQLLESGGGLVQPGGSLRLSCAASGFTFSSYGMHWVRQAPGKGLEWVSAINWNGGSTGYADSVKGRFTISRDNSKNTLYLQMNSLRAEDTAVYYCARGHHSGYDGRFFDYWGQGTLVTVSS

SEQ ID NO: 121 - protein, 111 aa, synthetic construct
  source 1..111: note = Human CCR8 specific benchmark antibody Ref#1 - VL region
QSVLTQPPSVSGAPGQRVTISCTGSSSNIGAGYNVHWYQQLPGTAPKLLIYTNNRRPSGVPDRFSGSKSGTSASLAISGLRSEDEADYYCAAWDASLSGWVFGGGTKLTVL

SEQ ID NO: 122 - protein, 8 aa, synthetic construct
  source 1..8: note = CDR sequence of anti-CCR8 antibody
GFTFNTYA

SEQ ID NO: 123 - protein, 10 aa, synthetic construct
  source 1..10: note = CDR sequence of anti-CCR8 antibody
IRSKSNNYAT

SEQ ID NO: 124 - protein, 19 aa, synthetic construct
  source 1..19: note = CDR sequence of anti-CCR8 antibody
VRGREAYYRYDGGYYAMDV

SEQ ID NO: 125 - protein, 11 aa, synthetic construct
  source 1..11: note = CDR sequence of anti-CCR8 antibody
KSLLHSNGNTY

SEQ ID NO: 126 - protein, 8 aa, synthetic construct
  source 1..8: note = CDR sequence of anti-CCR8 antibody
GFTFNAYA

SEQ ID NO: 127 - protein, 10 aa, synthetic construct
  source 1..10: note = CDR sequence of anti-CCR8 antibody
IRSKSNDYAT

SEQ ID NO: 128 - protein, 8 aa, synthetic construct
  source 1..8: note = CDR sequence of anti-CCR8 antibody
GYTFTEYT

SEQ ID NO: 129 - protein, 8 aa, synthetic construct
  source 1..8: note = CDR sequence of anti-CCR8 antibody
INPNNGNT

SEQ ID NO: 130 - protein, 16 aa, synthetic construct
  source 1..16: note = CDR sequence of anti-CCR8 antibody
ARVARSSGSGPYAMDY

SEQ ID NO: 131 - protein, 11 aa, synthetic construct
  source 1..11: note = CDR sequence of anti-CCR8 antibody
QSLLHTNGDTY

SEQ ID NO: 132 - protein, 107 aa, synthetic construct
  source 1..107: note = Variable domain sequence of anti-CCR8 antibody
DIQMTQSPSSLSASVGDRVTITCRASQDISNYLNWYQQKPDGTVKLLIYYTSRLHTGVPSRFSGSGSGTDYTLTISNLQQEDIATYFCQQGNKFPPTFGGGTKVEIN

SEQ ID NO: 133 - protein, 19 aa, synthetic construct
  source 1..19: note = CDR sequence of anti-CCR8 antibody
VRGREAYYRYDGGYYAMDY

SEQ ID NO: 134 - protein, 8 aa, synthetic construct
  source 1..8: note = CDR sequence of anti-CCR8 antibody
GFTFSNYR

SEQ ID NO: 135 - protein, 10 aa, synthetic construct
  source 1..10: note = CDR sequence of anti-CCR8 antibody
IKVKSDNYGA

SEQ ID NO: 136 - protein, 13 aa, synthetic construct
  source 1..13: note = CDR sequence of anti-CCR8 antibody
SSPTYPGSSGFAY

SEQ ID NO: 137 - protein, 6 aa, synthetic construct
  source 1..6: note = CDR sequence of anti-CCR8 antibody
QDISNY

SEQ ID NO: 138 - protein, 128 aa, synthetic construct
  source 1..128: note = Variable domain sequence of anti-CCR8 antibody
EVQLVESGGGLVQPGRSLRLSCTASGFTFNTYAMNWVRQAPGKGLEWVARIRSKSNNYATYYAASVKGRFTISRDDSKSIAYLQMNSLKTEDTAVYYCVRGREAYYRYDGGYYAMDVWGQGTTVTVSS

SEQ ID NO: 139 - protein, 112 aa, synthetic construct
  source 1..112: note = Variable domain sequence of anti-CCR8 antibody
DIVMTQAAPSLPVTPGESASISCRSSKSLLHSNGNTYLYWFLQKPGQSPQLLIYRMSNLASGVPDRFSGSGSGTAFTLKISRVEAEDVGVYYCMQHLEYPFTFGSGTKLEIK

SEQ ID NO: 140 - protein, 128 aa, synthetic construct
  source 1..128: note = Variable domain sequence of anti-CCR8 antibody
EVQLLESGGGLVQPGGSLRLSCAASGFTFNAYAMNWVRQAPGKGLEWVARIRSKSNDYATYYGDSVKGRFTISRDNSKNTLYLQMNSLRAEDTAVYYCVRGREAYYRYDGGYYAMDVWGQGTTVTVSS

SEQ ID NO: 141 - protein, 112 aa, synthetic construct
  source 1..112: note = Variable domain sequence of anti-CCR8 antibody
DIVMTQAAPSLSVTPGESASISCTSSKSLLHSNGNTYLYWFLQKPGQSPQLLIYRMSNLASGVPDRFSGSGSGTAFTLKISRVEAEDVGVYYCMQHREYPFTFGSGTKLEIK

SEQ ID NO: 142 - protein, 112 aa, synthetic construct
  source 1..112: note = Variable domain sequence of anti-CCR8 antibody
DIVMTQSPLSLPVTPGEPASISCRSSKSLLHSNGNTYLYWFLQKPGQSPQLLIYRVSNLASGVPDRFSGSGSGTDFTLKISRVEAEDVGVYYCMQHREYPFTFGQGTKLEIK

SEQ ID NO: 143 - protein, 123 aa, synthetic construct
  source 1..123: note = Variable domain sequence of anti-CCR8 antibody
QVQLVQSGPEVVKPGASVKVSCKTSGYTFTEYTIHWVRQAPGQSLEWVGGINPNNGNTRYDQKFQGRVTITRDKSASTAYMELRSLTSEDTAVYYCARVARSSGSGPYAMDYWGQGTTVTVSS

SEQ ID NO: 144 - protein, 112 aa, synthetic construct
  source 1..112: note = Variable domain sequence of anti-CCR8 antibody
DVVMTQSPLSLPVTLGDQASISCRCSQSLLHTNGDTYLHWYLQRPGQSPRLLIYKVSNRFSGVPDRFSGSGSGTDFTLKISRVEAEDVGVYFCSQSTHVPYTFGGGTKVEIK

SEQ ID NO: 145 - protein, 123 aa, synthetic construct
  source 1..123: note = Variable domain sequence of anti-CCR8 antibody
QVQLQQSGPEVVKPGASVKVSCKTSGYTFTEYTIHWVRQSHGQSLEWVGGINPNNGNTRYDQKFKGRVTITIDKSSSTAYMELRSLTSEDTAVYYCARVARSSGSGPYAMDYWGQGTTVTVSS

SEQ ID NO: 146 - protein, 122 aa, synthetic construct
  source 1..122: note = Variable domain sequence of anti-CCR8 antibody
QVQLVESGGGVVQPGRSLRLSCAASGFTFSNYRMHWVRQAPGKGLEWIAVIKVKSDNYGANYADSVKGRFTISRDNSKNTLYLQMNSLRAEDTAVYYCSSPTYPGSSGFAYWGQGTLVTVSS

SEQ ID NO: 147 - protein, 128 aa, synthetic construct
  source 1..128: note = Variable domain sequence of anti-CCR8 antibody
EVQLVESGGGLVQPTGSLRLSCAASGFTFNAYAMNWVRQAPGKGLEWVARIRSKSNDYATYYGDSVKDRFTISRDDSQSMLYLQMNNLKTEDTAMYYCVRGREAYYRYDGGYYAMDYWGQGTSVTVSS

SEQ ID NO: 148 - protein, 112 aa, synthetic construct
  source 1..112: note = Variable domain sequence of anti-CCR8 antibody
DIVMTQAAPSVSVTPGESVSISCTSSKSLLHSNGNTYLYWFLQRPGQSPQLLIYRMSNLASGVPDRFSGSGSGTAFTLRISRVEAEDVGVYYCMQHREYPFTFGSGTKLEIK

SEQ ID NO: 149 - protein, 123 aa, synthetic construct
  source 1..123: note = Variable domain sequence of anti-CCR8 antibody
EVQLQQSGPELVKPGASVKISCKTSGYTFTEYTIHWVKQSHGESLEWVGGINPNNGNTRYDQKFKGKATLTIDKSSSPAYMELRSLTSEDSAVYYCARVARSSGSGPYAMDYWGQGTSVTVSS

SEQ ID NO: 150 - protein, 112 aa, synthetic construct
  source 1..112: note = Variable domain sequence of anti-CCR8 antibody
DVVMTQTPLSLPVSLGDQASISCRCTQSLLHTNGDTYLHWYLQKPGQSPKLLIYKVSNRFSGVPDRFSGSGSGTDFTLKISRVEAEDLGVYFCSQSTHVPYTFGGGTKLEIK

SEQ ID NO: 151 - protein, 128 aa, synthetic construct
  source 1..128: note = Variable domain sequence of anti-CCR8 antibody
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRGREAYYRYDGGYYAMDYWGQGTSVTVSS

SEQ ID NO: 152 - protein, 112 aa, synthetic construct
  source 1..112: note = Variable domain sequence of anti-CCR8 antibody
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 153 - protein, 122 aa, synthetic construct
  source 1..122: note = Variable domain sequence of anti-CCR8 antibody
QVQLVETGGGLVRPGNSLKLSCVTSGFTFSNYRMHWLRQPPGKRLEWIAVIKVKSDNYGANYAESVKGRFTISRDDSKSSVYLQVNRLREEDTATYYCSSPTYPGSSGFAYWGQGTLVTVSA

SEQ ID NO: 154 - protein, 107 aa, synthetic construct
  source 1..107: note = Variable domain sequence of anti-CCR8 antibody
DIQMTQTTSSLSASLGDRVTISCRASQDISNYLNWYQQKPDGTVKLLIYYTSRLHSGVPSRFSGSGSGTDYSLTISNLEQEDIATYFCQQGNKFPPTFGGGTKLEIN

SEQ ID NO: 155 - protein, 119 aa, synthetic construct
  source 1..119: note = Human CCR8 specific benchmark antibody Ref#2 - VH region
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARAVRNRFRFDYWGQGTLVTVSS

SEQ ID NO: 156 - protein, 111 aa, synthetic construct
  source 1..111: note = Human CCR8 specific benchmark antibody Ref#2 - VL region
QSALTQPASVSGSPGQSITISCTGTSSDVGSYNLVSWYQQHPGKAPKLMIYEVSKRPSGVSNRFSGSKSGNTASLTISGLQAEDEADYYCSSYAGSSTFVVFGGGTKLTVL

SEQ ID NO: 157 - protein, 123 aa, synthetic construct
  source 1..123: note = Human CCR8 specific benchmark antibody Ref#3 - VH region
EVQLVESGGGLVQPGGSLKLSCAASGFTFNTYAMNWVRQASGKGLEWVARIRSKANNYATYYADSVKDRFTISRDDSKNTLYLQMNNLKTEDTAVYYCVRDRSRGEDYAMDYWGQGTLVTVSS

SEQ ID NO: 158 - protein, 112 aa, synthetic construct
  source 1..112: note = Human CCR8 specific benchmark antibody Ref#3 - VL region
DIVMTQSPLSLPVTPGEPASISCRSSKSLLHSNANTYLYWFLQKPGQSPQLLIYRMSNLASGVPDRFSGSGSGTAFTLKISRVEAEDVGVYYCMQHLEYPFTFGQGTKLEIK

SEQ ID NO: 159 - protein, 457 aa, synthetic construct
  source 1..457: note = Heavy chain sequence of anti-CCR8 antibody
EVQLVESGGGLVQPGRSLRLSCTASGFTFNTYAMNWVRQAPGKGLEWVARIRSKSNNYATYYAASVKGRFTISRDDSKSIAYLQMNSLKTEDTAVYYCVRGREAYYRYDGGYYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 219 aa, synthetic construct
  source 1..219: note = Light chain sequence of anti-CCR8 antibody
DIVMTQAAPSLPVTPGESASISCRSSKSLLHSNGNTYLYWFLQKPGQSPQLLIYRMSNLASGVPDRFSGSGSGTAFTLKISRVEAEDVGVYYCMQHLEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 457 aa, synthetic construct
  source 1..457: note = Heavy chain sequence of anti-CCR8 antibody
EVQLLESGGGLVQPGGSLRLSCAASGFTFNAYAMNWVRQAPGKGLEWVARIRSKSNDYATYYGDSVKGRFTISRDNSKNTLYLQMNSLRAEDTAVYYCVRGREAYYRYDGGYYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 219 aa, synthetic construct
  source 1..219: note = Light chain sequence of anti-CCR8 antibody
DIVMTQAAPSLSVTPGESASISCTSSKSLLHSNGNTYLYWFLQKPGQSPQLLIYRMSNLASGVPDRFSGSGSGTAFTLKISRVEAEDVGVYYCMQHREYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 457 aa, synthetic construct
  source 1..457: note = Heavy chain sequence of anti-CCR8 antibody
EVQLLESGGGLVQPGGSLRLSCAASGFTFNAYAMNWVRQAPGKGLEWVARIRSKSNDYATYYGDSVKGRFTISRDNSKNTLYLQMNSLRAEDTAVYYCVRGREAYYRYDGGYYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 219 aa, synthetic construct
  source 1..219: note = Light chain sequence of anti-CCR8 antibody
DIVMTQSPLSLPVTPGEPASISCRSSKSLLHSNGNTYLYWFLQKPGQSPQLLIYRVSNLASGVPDRFSGSGSGTDFTLKISRVEAEDVGVYYCMQHREYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 452 aa, synthetic construct
  source 1..452: note = Heavy chain sequence of anti-CCR8 antibody
QVQLVQSGPEVVKPGASVKVSCKTSGYTFTEYTIHWVRQAPGQSLEWVGGINPNNGNTRYDQKFQGRVTITRDKSASTAYMELRSLTSEDTAVYYCARVARSSGSGPYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 219 aa, synthetic construct
  source 1..219: note = Light chain sequence of anti-CCR8 antibody
DVVMTQSPLSLPVTLGDQASISCRCSQSLLHTNGDTYLHWYLQRPGQSPRLLIYKVSNRFSGVPDRFSGSGSGTDFTLKISRVEAEDVGVYFCSQSTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 452 aa, synthetic construct
  source 1..452: note = Heavy chain sequence of anti-CCR8 antibody
QVQLQQSGPEVVKPGASVKVSCKTSGYTFTEYTIHWVRQSHGQSLEWVGGINPNNGNTRYDQKFKGRVTITIDKSSSTAYMELRSLTSEDTAVYYCARVARSSGSGPYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 451 aa, synthetic construct
  source 1..451: note = Heavy chain sequence of anti-CCR8 antibody
QVQLVESGGGVVQPGRSLRLSCAASGFTFSNYRMHWVRQAPGKGLEWIAVIKVKSDNYGANYADSVKGRFTISRDNSKNTLYLQMNSLRAEDTAVYYCSSPTYPGSSGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 214 aa, synthetic construct
  source 1..214: note = Light chain sequence of anti-CCR8 antibody
DIQMTQSPSSLSASVGDRVTITCRASQDISNYLNWYQQKPDGTVKLLIYYTSRLHTGVPSRFSGSGSGTDYTLTISNLQQEDIATYFCQQGNKFPPTFGGGTKVEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 457 aa, synthetic construct
  source 1..457: note = Heavy chain sequence of anti-CCR8 antibody
EVQLVESGGGLVQPTGSLRLSCAASGFTFNAYAMNWVRQAPGKGLEWVARIRSKSNDYATYYGDSVKDRFTISRDDSQSMLYLQMNNLKTEDTAMYYCVRGREAYYRYDGGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 219 aa, synthetic construct
  source 1..219: note = Light chain sequence of anti-CCR8 antibody
DIVMTQAAPSVSVTPGESVSISCTSSKSLLHSNGNTYLYWFLQRPGQSPQLLIYRMSNLASGVPDRFSGSGSGTAFTLRISRVEAEDVGVYYCMQHREYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 457 aa, synthetic construct
  source 1..457: note = Heavy chain sequence of anti-CCR8 antibody
EVQLVESGGGLVQPTGSLRLSCAASGFTFNAYAMNWVRQAPGKGLEWVARIRSKSNDYATYYGDSVKDRFTISRDDSQSMLYLQMNNLKTEDTAMYYCVRGREAYYRYDGGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 457 aa, synthetic construct
  source 1..457: note = Heavy chain sequence of anti-CCR8 antibody
EVQLVESGGGLVQPTGSLRLSCAASGFTFNAYAMNWVRQAPGKGLEWVARIRSKSNDYATYYGDSVKDRFTISRDDSQSMLYLQMNNLKTEDTAMYYCVRGREAYYRYDGGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 457 aa, synthetic construct
  source 1..457: note = Heavy chain sequence of anti-CCR8 antibody
EVQLVESGGGLVQPTGSLRLSCAASGFTFNAYAMNWVRQAPGKGLEWVARIRSKSNDYATYYGDSVKDRFTISRDDSQSMLYLQMNNLKTEDTAMYYCVRGREAYYRYDGGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPG

SEQ ID NO: 175 - protein, 457 aa, synthetic construct
  source 1..457: note = Heavy chain sequence of anti-CCR8 antibody
EVQLVESGGGLVQPTGSLRLSCAASGFTFNAYAMNWVRQAPGKGLEWVARIRSKSNDYATYYGDSVKDRFTISRDDSQSMLYLQMNNLKTEDTAMYYCVRGREAYYRYDGGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNWALPAPISKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPG

SEQ ID NO: 176 - protein, 452 aa, synthetic construct
  source 1..452: note = Heavy chain sequence of anti-CCR8 antibody
EVQLQQSGPELVKPGASVKISCKTSGYTFTEYTIHWVKQSHGESLEWVGGINPNNGNTRYDQKFKGKATLTIDKSSSPAYMELRSLTSEDSAVYYCARVARSSGSGPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 219 aa, synthetic construct
  source 1..219: note = Light chain sequence of anti-CCR8 antibody
DVVMTQTPLSLPVSLGDQASISCRCTQSLLHTNGDTYLHWYLQKPGQSPKLLIYKVSNRFSGVPDRFSGSGSGTDFTLKISRVEAEDLGVYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 452 aa, synthetic construct
  source 1..452: note = Heavy chain sequence of anti-CCR8 antibody
EVQLQQSGPELVKPGASVKISCKTSGYTFTEYTIHWVKQSHGESLEWVGGINPNNGNTRYDQKFKGKATLTIDKSSSPAYMELRSLTSEDSAVYYCARVARSSGSGPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 452 aa, synthetic construct
  source 1..452: note = Heavy chain sequence of anti-CCR8 antibody
EVQLQQSGPELVKPGASVKISCKTSGYTFTEYTIHWVKQSHGESLEWVGGINPNNGNTRYDQKFKGKATLTIDKSSSPAYMELRSLTSEDSAVYYCARVARSSGSGPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 180 - protein, 452 aa, synthetic construct
  source 1..452: note = Heavy chain sequence of anti-CCR8 antibody
EVQLQQSGPELVKPGASVKISCKTSGYTFTEYTIHWVKQSHGESLEWVGGINPNNGNTRYDQKFKGKATLTIDKSSSPAYMELRSLTSEDSAVYYCARVARSSGSGPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPG

SEQ ID NO: 181 - protein, 452 aa, synthetic construct
  source 1..452: note = Heavy chain sequence of anti-CCR8 antibody
EVQLQQSGPELVKPGASVKISCKTSGYTFTEYTIHWVKQSHGESLEWVGGINPNNGNTRYDQKFKGKATLTIDKSSSPAYMELRSLTSEDSAVYYCARVARSSGSGPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNWALPAPISKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPG

SEQ ID NO: 182 - protein, 457 aa, synthetic construct
  source 1..457: note = Heavy chain sequence of anti-CCR8 antibody
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRGREAYYRYDGGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 183 - protein, 219 aa, synthetic construct
  source 1..219: note = Light chain sequence of anti-CCR8 antibody
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 457 aa, synthetic construct
  source 1..457: note = Heavy chain sequence of anti-CCR8 antibody
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRGREAYYRYDGGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 185 - protein, 457 aa, synthetic construct
  source 1..457: note = Heavy chain sequence of anti-CCR8 antibody
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRGREAYYRYDGGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 186 - protein, 457 aa, synthetic construct
  source 1..457: note = Heavy chain sequence of anti-CCR8 antibody
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRGREAYYRYDGGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPG

SEQ ID NO: 187 - protein, 457 aa, synthetic construct
  source 1..457: note = Heavy chain sequence of anti-CCR8 antibody
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRGREAYYRYDGGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNWALPAPISKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPG

SEQ ID NO: 188 - protein, 451 aa, synthetic construct
  source 1..451: note = Heavy chain sequence of anti-CCR8 antibody
QVQLVETGGGLVRPGNSLKLSCVTSGFTFSNYRMHWLRQPPGKRLEWIAVIKVKSDNYGANYAESVKGRFTISRDDSKSSVYLQVNRLREEDTATYYCSSPTYPGSSGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 189 - protein, 214 aa, synthetic construct
  source 1..214: note = Light chain sequence of anti-CCR8 antibody
DIQMTQTTSSLSASLGDRVTISCRASQDISNYLNWYQQKPDGTVKLLIYYTSRLHSGVPSRFSGSGSGTDYSLTISNLEQEDIATYFCQQGNKFPPTFGGGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 190 - protein, 451 aa, synthetic construct
  source 1..451: note = Heavy chain sequence of anti-CCR8 antibody
QVQLVETGGGLVRPGNSLKLSCVTSGFTFSNYRMHWLRQPPGKRLEWIAVIKVKSDNYGANYAESVKGRFTISRDDSKSSVYLQVNRLREEDTATYYCSSPTYPGSSGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 191 - protein, 451 aa, synthetic construct
  source 1..451: note = Heavy chain sequence of anti-CCR8 antibody
QVQLVETGGGLVRPGNSLKLSCVTSGFTFSNYRMHWLRQPPGKRLEWIAVIKVKSDNYGANYAESVKGRFTISRDDSKSSVYLQVNRLREEDTATYYCSSPTYPGSSGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 192 - protein, 451 aa, synthetic construct
  source 1..451: note = Heavy chain sequence of anti-CCR8 antibody
QVQLVETGGGLVRPGNSLKLSCVTSGFTFSNYRMHWLRQPPGKRLEWIAVIKVKSDNYGANYAESVKGRFTISRDDSKSSVYLQVNRLREEDTATYYCSSPTYPGSSGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPG

SEQ ID NO: 193 - protein, 451 aa, synthetic construct
  source 1..451: note = Heavy chain sequence of anti-CCR8 antibody
QVQLVETGGGLVRPGNSLKLSCVTSGFTFSNYRMHWLRQPPGKRLEWIAVIKVKSDNYGANYAESVKGRFTISRDDSKSSVYLQVNRLREEDTATYYCSSPTYPGSSGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNWALPAPISKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPG

(19) United States
(12) Patent Application Publication
(10) Pub. No.: US 2026/0132208 A1
(43) Pub. Date: May 14, 2026

(54) ANTI-CCR8 MONOCLONAL ANTIBODIES AND THEIR THERAPEUTIC USE

(71) Applicant: DOMAIN THERAPEUTICS, Illkirch-Graffenstaden (FR)

(72) Inventors: Christel FRANCHET, Illkirch-Graffenstaden (FR); María Delores GARCÍA FERNÁNDEZ, Illkirch-Graffenstaden (FR); Malaury SCHAPPLER, Illkirch-Graffenstaden (FR); Nathalie LENNE, Illkirch-Graffenstaden (FR); Orphée Blanchard, Illkirch-Graffenstaden (FR); Xavier LEROY, Illkirch-Graffenstaden (FR); Claudine Vermot-Desroches, Illkirch-Graffenstaden (FR); Julien ISOARD, Illkirch-Graffenstaden (FR); Yannick NIZET, Illkirch-Graffenstaden (FR)

(73) Assignee: Domain Therapeutics, Illkirch-Graffenstaden (FR)

(21) Appl. No.: 19/113,554

(22) PCT Filed: Sep. 21, 2023
(86) PCT No.: PCT/EP2023/076155

§ 371 Date: Mar. 20, 2025

(30) Foreign Application Priority Data
Sep. 21, 2022 (EP) 22306389.2

(51) Int. Cl. C07K 16/28; A61K 35/17; A61K 40/11; A61K 40/31; A61K 40/42; A61K 45/06; A61P 35/00

(52) U.S. Cl. CPC C07K 16/2866; A61K 35/17; A61K 40/11; A61K 40/31; A61K 40/4202; A61K 45/06; A61P 35/00; A61K 2239/13; C07K 2317/33; C07K 2317/41; C07K 2317/52; C07K 2317/565; C07K 2317/622; C07K 2317/72; C07K 2317/732; C07K 2317/734; C07K 2317/76

(57) ABSTRACT

The present invention relates to a monoclonal antibody or an antigen-binding fragment thereof, which specifically binds to human CCR8 and is an antagonist, particularly an insurmountable antagonist, of the CCL1-CCR8 signaling pathway. The invention also relates to the antibody or antigen-binding fragment for use in therapy, particularly in the treatment of cancer.

The present invention relates to a monoclonal antibody or an antigen-binding fragment thereof, which specifically binds to human CCR8 and is an antagonist, particularly an insurmountable antagonist, of the CCL1-CCR8 signaling pathway. The invention also relates to the antibody or antigen-binding fragment for use in therapy, particularly in the treatment of cancer.

Chemokine (C-C motif) receptor 8 (CCR8) (which is also called CKRL1, CMKBR8, or CMKBRL2) belongs to the G protein-coupled receptor (GPCR) family. CCR8 is primarily expressed on tumor regulatory T (Treg) cells, a type of immuno-suppressive cell found in the tumor microenvironment. Regulatory T (Treg) cells are one of the integral components of the adaptive immune system whereby they contribute to maintaining tolerance to self-antigens and preventing auto-immune diseases (Vignali D A A et al., Nature Reviews Immunology, 2008, 8(7): 523-32, doi: 10.1038/nri2343).

However, Treg cells are also found to be highly enriched in the tumor microenvironment of many different cancers (Colombo M P et al., Nature Reviews Cancer, 2007, 7(11): 880-7, doi: 10.1038/nrc2250; Nishikawa H et al., Current Opinion in Immunology, 2014, 27: 1-7, doi: 10.1016/j.coi.2013.12.005). Based on the transcriptional landscape of tumor infiltrating T regulatory cells (TITR), it has been found that CCR8 positive TITR were highly immune suppressive and defined as a specific signature molecule (De Simone M et al., Immunity, 2016, 45(5): 1135-47, doi: 10.1016/j.immuni.2016.10.021).

Multiple strategies have been proposed to modulate Treg cells in the tumor microenvironment to enhance therapeutic benefit (Elpek K G et al., The Journal of Immunology, 2007, 178(11): 6840-8, doi: 10.4049/jimmunol. 178.11.6840). In the tumor microenvironment, Treg cells contribute to immune escape by reducing tumor-associated antigen (TAA)-specific T-cell immunity, thereby preventing effective anti-tumor activity. High tumor infiltration by Tregs is hence often associated with an invasive phenotype and poor prognosis in cancer patients (Shang B et al., Scientific Reports, 2015, 5:15179, doi: 10.1038/srep15179; Plitas G et al., Immunity, 2016, 45(5): 1122-34, doi: 10.1016/j.immuni.2016.10.032).

Due to the high and relatively specific expression of CCR8 on tumor infiltrating Tregs, CCR8 represents an attractive immunotherapeutic target to inhibit Treg cells trafficking triggered with CCL1 to tumor sites without disturbing recruitment of other effector T cells that do not express CCR8. Moreover, the use of depleting anti-CCR8 mAb with enhanced cytotoxic activity (i.e., ADCC, CDC, ADCP) can also reduce the number of CCR8 immuno-suppressive cells in a tumor. Up to date, several monoclonal antibodies against CCR8 have been used for the modulation and depletion of this Treg population in the treatment of cancer.

Indeed, CCR8 expression on tumor resident Tregs has been reported in various types of human cancers, including breast cancer, bladder cancer, colorectal cancer, lung cancer, pancreatic cancer, melanoma, and angiosarcoma (Tanaka A et al., Eur J Immunol, 2019, 49(8): 1140-6, doi: 10.1002/eji.201847659; De Simone M et al., Immunity, 2016, 45(5): 1135-47, doi: 10.1016/j.immuni.2016.10.021; Plitas G et al., Immunity, 2016, 45(5): 1122-34, doi: 10.1016/j.immuni.2016.10.032; Wang T et al., Cancer Immunol Immunother, 2020, 69(9): 1855-67, doi: 10.1007/s00262-020-02583-y; Campbell J R et al., Cancer Res, 2021, 81(11): 2983-94, doi: 10.1158/0008-5472.CAN-20-3585; Magnuson A M et al., Proc Natl Acad Sci USA, 2018, 115(45): E10672-81, doi: 10.1073/pnas. 1810580115; Tirosh I et al., Science, 2016, 352(6282): 189-96, doi: 10.1126/science.aad0501; Islam S A et al., J Exp Med, 2013, 210(10): 1889-98, doi: 10.1084/jem.20130240). Interestingly, in patients with pancreatic cancer, high CCR8+ Treg numbers correlated with more advanced stages of the disease and a decreased overall survival (Yi G et al., Science Bulletin, 2018, 63(15): 972-81, doi: 10.1016/j.scib.2018.05.028). These findings further highlight the broad clinical applicability of CCR8 blocking and/or depleting antibodies in the treatment of a variety of cancers.

Moreover, CCR8 expression has also been reported on T tumor cells, such as, e.g., in cutaneous T-cell lymphomas (CTCL) (Giustiniani J et al., Blood Adv, 2022, 6(11): 3507-12, doi: 10.1182/bloodadvances.2021006512). Mycosis fungoides (MF) and Sézary syndrome (SS) are the most frequent cutaneous T-cell lymphomas. It has been revealed that CCR8 is overexpressed at the cell surface of CTCL peripheral blood tumor cells and is involved in Sézary cell activation and proliferation (Giustiniani J et al., loc. cit.). An increased CCR8 expression was furthermore detected on adult T-cell leukemia/lymphoma patients (Zheng D et al., Front Immunol, 2022, 13:808347, doi: 10.3389/fimmu.2022.808347). Adult T-cell leukemia/lymphoma (ATLL) and peripheral T cell lymphoma (PTCL) are major subtypes of T-cell lymphoma (Hotta T, Hematology, 2005, 10 Suppl 1:193-6, doi: 10.1080/10245330512331390393). ATLL is a malignancy of mature T lymphocytes that is triggered by human T-cell lymphotropic virus type I (HTLV-1) (Uchiyama T et al., Blood, 1977, 50(3): 481-92, doi: 10.1182/blood.V50.3.481.481; Ishitsuka K et al., Lancet Oncol, 2014, 15(11): e517-26, doi: 10.1016/S1470-2045(14)70202-5). Therefore, CCR8 is considered to be a therapeutic target in distinct aggressive T-cell lymphoma subtypes.

Recent studies highlight the clinical applicability of CCR8 blocking and/or depleting antibodies, either as a single agent or in combination with other forms of cancer treatment (e.g., radiotherapy, chemotherapies and/or immunotherapies), and also of corresponding antibody-drug conjugates (ADCs). In particular, different combination approaches based on immunotherapies are under investigation, such as combinations with checkpoint inhibitors, costimulatory molecules, kinase inhibitors, chimeric antigen receptor (CAR) cell-based therapies, and cancer vaccines. Combining multiple immunotherapies for cancer will be critical for improving the therapeutic outcome for future clinical trials.

Notably, a synergistic antitumor effect has been reported for the combination of anti-CCR8 monoclonal antibodies with anti-PD-1 monoclonal antibodies (Van Damme H et al., J Immunother Cancer, 2021, 9(2): e001749, doi: 10.1136/jitc-2020-001749). The reduction in tumor growth of the combination therapy could be attributed to a more immunogenic tumor microenvironment (TME) rich in effector CD8+ T cells. This altered balance between suppressive Tregs and effector CD8+ T cells has been reported to be crucial for an effective antitumor immune response (Van Damme H et al., loc. cit.; Tanaka A et al., Eur J Immunol, 2019, 49(8): 1140-6, doi: 10.1002/eji.201847659).

Therapeutic approaches combining the use of anti-CCR8 monoclonal antibodies with cancer vaccines have also proven to be of particular interest. For example, initial in vivo evidence was provided that a Listeria-based cancer vaccine immunotherapy targeting the classical AH1 tumor-associated antigen can be coupled with a CCR8 monoclonal antibody, supporting the use of this combination strategy in future clinical trials (Villarreal D O et al., Cancer Res, 2018, 78(18): 5340-8, doi: 10.1158/0008-5472.CAN-18-1119).

Moreover, chimeric antigen receptor (CAR) T cells have been successfully used in the therapy of B cell leukemia and lymphoma, but still have many challenges in their use for treating T cell malignancies and also for solid tumors, such as the lack of unique tumor antigens, their limitation of T cell expansion, and the need for third-party donors or genome editing (Benmebarek M R et al., Int J Mol Sci, 2019, 20(6): 1283, doi: 10.3390/ijms20061283). CAR T cell therapy often remains ineffective in solid tumors, due largely to poor infiltration and T cell suppression at the tumor site. Treg cells suppress the immune response via inhibitory factors such as transforming growth factor-beta (TGF-β) (Plitas G et al., Immunity, 2016, 45(5): 1122-34, doi: 10.1016/j.immuni.2016.10.032; Barsheshet Y et al., Proc Natl Acad Sci USA, 2017, 114(23): 6086-91, doi: 10.1073/pnas. 1621280114). Anti-CCR8 CAR T cells have further been shown to prolong survival in ATLL tumor-bearing mouse models without impairing T cell expansion (Zheng D et al., Front Immunol, 2022, 13:808347, doi: 10.3389/fimmu.2022.808347). Recent studies have demonstrated that CCR8-engineered T cells improve CAR T cell therapy for pancreatic cancer (Cadilha B L et al., Sci Adv, 2021, 7(24): eabi5781, doi: 10.1126/sciadv.abi5781). The therapeutic potential of this approach could extend to other Treg-rich solid tumor entities where limited infiltration into the tumor and intratumoral T cell proliferation prevent therapeutic success.

Further reports point to possible combination therapies using anti-CCR8 monoclonal antibodies together with Treg depleting monoclonal antibodies targeting, e.g., CD25 (Onizuka S et al., Cancer Res, 1999, 59(13): 3128-33; Rech A J et al., Sci Transl Med, 2012, 4(134): 134ra62, doi: 10.1126/scitranslmed.3003330; Shimizu J et al., J Immunol, 1999, 163(10): 5211-8) or CCR4 (Sugiyama D et al., Proc Natl Acad Sci USA, 2013, 110(44): 17945-50, doi: 10.1073/pnas.1316796110).

Anti-CCR8 monoclonal antibodies, both as a monotherapy and in the context of co-therapeutic approaches, have thus been proposed for the treatment of a wide range of cancers.

Yet, antibodies targeting CCR8 may also be used for other therapeutic applications beyond the treatment of cancer. Human CCR8 is expressed only in lymphoid organs and in the thymus (Napolitano M et al., J Immunol, 1996, 157(7): 2759-63; Samson M et al., Eur J Immunol, 1996, 26(12): 3021-8, doi: 10.1002/eji. 1830261230; Zaballos A et al., Biochem Biophys Res Commun, 1996, 227(3): 846-53, doi: 10.1006/bbrc. 1996.1595). CCR8 is not only expressed on T regs but also by a subset of memory CD4+ T cells enriched in Th2 cells (Chensue S W et al., J Exp Med, 2001, 193(5): 573-84, doi: 10.1084/jem.193.5.573). NKT cells also express CCR4 and CCR8 (Harner S et al., PLOS One, 2011, 6(1): e15714, doi: 10.1371/journal.pone.0015714). The presence of CCR8 on inflammatory macrophages in human chronic obstructive pulmonary disease (COPD) has also been reported (Reimer M K et al., Clin Vaccine Immunol, 2011, 18(12): 2050-9, doi: 10.1128/CVI.05275-11). In vitro studies have demonstrated that CCR8 expression is regulated by T cell receptor (TCR) engagement and the skin tissue microenvironment (McCully M L et al., J Immunol, 2018, 200(5): 1639-50, doi: 10.4049/jimmunol.1701377). The CCR8 expression on these different immune cell subsets suggests future therapeutic approaches to target CCR8 for immune-oncology, autoimmunity, inflammation or in the context of bacterial load.

Antibodies that recognize CCR8 and corresponding therapeutic applications have been described in the literature (see, e.g., WO 2007/044756, WO 2018/112032, WO 2018/112033, WO 2018/181425 or EP 3 431 105, WO 2020/138489 or EP 3 903 817, WO 2021/142002 or US 2021/0238292, WO 2021/152186, WO 2021/194942, WO 2021/260210, WO 2022/003156, WO 2022/042690, or WO 2022/078277).

CCL1, one of the ligands of CCR8, acts as a potent attractant for a subset of T-regulatory (Treg) cells (Barsheshet Y et al., Proc Natl Acad Sci USA, 2017, 114(23): 6086-91, doi: 10.1073/pnas. 1621280114). CCL1 is produced by activated monocytes/macrophages and T-lymphocytes (Selvan R S et al., Eur J Immunol, 1997, 27(3): 687-94, doi: 10.1002/eji. 1830270317), from fibroblasts (Yeh C R et al., Am J Cancer Res, 2015, 5(3): 1146-57) and Th2 cells (Zingoni A et al., J Immunol, 1998, 161(2): 547-51). In cancer, the CCL1-CCR8 autocrine loop has been shown to have a protective function by enabling lymphoma and T cell leukemia cells to avoid apoptosis in vitro (Van Snick J et al., J Immunol, 1996, 157(6): 2570-6; Ruckes T et al., Blood, 2001, 98(4): 1150-9, doi: 10.1182/blood.v98.4.1150) and to play a role in T cell transformation (Tamgüney G et al., Oncogene, 2004, 23(52): 8475-85, doi: 10.1038/sj.onc.1207903). As a cytokine, it has the capacity of affecting Tregs themselves in an autocrine manner, or other cells in the vicinity of Tregs, in a paracrine manner. Autocrine effects are likely because Tregs express the receptor for CCL1, i.e. CCR8, and migrate in response to ligand binding (De Simone M et al., Immunity, 2016, 45(5): 1135-47, doi: 10.1016/j.immuni.2016.10.021). Interestingly, Tregs that migrate in response to CCL1 are also more suppressive (De Simone M et al., Immunity, 2016, 45(5): 1135-47, doi: 10.1016/j.immuni.2016.10.021).

Due to the key functions of CCL1, the present inventors considered it to be highly advantageous to preserve the antagonistic effect of anti-CCR8 monoclonal antibodies (mAbs) on CCL1 induced signaling pathways whatever the CCL1 concentration. To address this key feature, the present inventors selected therapeutic anti-CCR8 mAbs based on the criterion of having an insurmountable antagonistic activity.

Thus, surprisingly, the inventors obtained a number of antibodies showing insurmountable antagonism activity, which can be the result of allosteric or orthosteric blockade. The insurmountable mAb antagonists of the present invention have indeed the capacity to depress the maximal response of CCR8 to CCL1, irrespective of CCL1 concentrations. Consequently, the antibodies according to the present invention allow broader pharmacological responses in various physiopathological conditions where different CCL1 concentrations apply. Due to the insurmountability of their antagonist activities, these mAbs are less dependent on CCL1 concentrations, which renders them highly advantageous for therapeutic use. The present inventors have thus provided insurmountable antagonist antibodies allowing to identify a novel strategy to block Treg conversion and suppressive function, without compromising the antagonistic mAb activity even in the presence of various CCL1 concentrations, with highest potential therapeutic benefit.

Accordingly, the present invention provides a monoclonal antibody or an antigen-binding fragment thereof, wherein said antibody or antigen-binding fragment specifically binds to human CCR8 and is an antagonist of the CCL1-CCR8 signaling pathway. Preferably, the antibody or antigen-binding fragment is an insurmountable antagonist of the CCL1-CCR8 signaling pathway.

In the context of the present invention, antibodies that specifically bind to human CCR8 have been generated, which exhibit highly advantageous properties. In particular, the antibodies (and antigen-binding fragments) provided herein act as insurmountable antagonists of the CCL1-CCR8 signaling pathway, as also demonstrated in Example 14. Due to this insurmountable antagonist activity, the antibodies (and antigen-binding fragments) according to the invention exert a particularly potent, sustained and uniform therapeutic effect, even in the presence of high concentrations of 10 the endogenous ligand CCL1, which renders them highly advantageous for use in therapy, particularly for the treatment of cancer. Indeed, this insurmountability makes the antibody or antigen-binding fragment resistant to various concentrations of the endogenous ligand of CCR8 present in the tumor-microenvironment, and therefore less dependent on the type of cancer/tumor and the patient to be treated. The advantages of insurmountable antagonists in the context of anti-cancer or anti-inflammatory drugs have also been affirmed in the literature (see, e.g., Houthuys E et al, Cancer Res, 2018, 78(13_Supplement): LB-291, doi: 10.1158/1538-7445.AM2018-LB-291; Ortiz Zacarias N V et al, J Med Chem, 2019, 62(24): 11035-53, doi: 10.1021/acs.jmedchem.9b00742; Richard-Bildstein S et al, J Med Chem, 2020, 63(24): 15864-82, doi: 10.1021/acs.jmedchem.0c01588).

The antibodies (and antigen-binding antibody fragments) according to the present invention are furthermore advantageous in that they exert a potent antagonistic activity, particularly an insurmountable antagonist activity, on the CCL1-CCR8 signaling pathway not only at a neutral physiological pH of 7.4 but also at an acidic pH as it is typically found in the tumor microenvironment (TME). Thus, as reported in the literature, the interstitial extracellular pH (pHe) in tumors is rather acidic (pH 6.2-6.9) compared with normal tissues (pH 7.3-7.4) (Griffiths J R, Br J Cancer, 1991, 64(3): 425-27, doi: 10.1038/bjc.1991.326; Wike-Hooley J L et al., Radiother Oncol, 1984, 2(4): 343-66, doi: 10.1016/s0167-8140(84)80077-8), which is caused in part by the overstimulation of several ion transporters, such as Na⁺/H⁺ exchanger (NHE; Griffiths J R, loc. cit.), Na⁺-dependent and independent HCO₃⁻/Cl⁻ exchangers and the mono-carboxylate transporter, which increase H⁺ ions in the extracellular space and acidify the pHe in tumors (Madshus I_{H}, Biochem J, 1988, 250(1): 1-8, doi: 10.1042/bj2500001). In addition, most cancer cells rely on aerobic glycolysis to generate the energy needed for cellular processes, a phenomenon named the Warburg effect, which is one of the 30 principal factors inducing an acidic TME in the tumor extracellular region (Warburg O, Science, 1956, 123(3191): 309-14, doi: 10.1126/science. 123.3191.309). It has also been reported that the acidic TME is related to tumor progression and metastasis (Cardone R A et al., Nat Rev Cancer, 2005, 5(10): 786-95, doi: 10.1038/nrc1713; Xie R et al., Oncol Rep, 2017, 37(3): 1451-60, doi: 10.3892/or.2017.5386). Moreover, the acidic TME impairs the responses of tumors to some anti-cancer chemotherapies (Mahoney B P et al., Biochem Pharmacol, 2003, 66(7): 1207-18, doi: 10.1016/s0006-2952(03)00467-2). It is therefore highly advantageous for therapeutic antibodies to have preserved or even preferential activity under the acidic pH conditions of the TME. Remarkably, the antibodies (and antigen-binding fragments) provided in accordance with the present invention have been found to exert highly potent antagonist activity even at the acidic pH that is present in the TME, which makes these antibodies especially well suited for the therapeutic treatment of cancer. In particular, the antibodies (and antigen-binding fragments) according 40 to the invention have been found to exhibit potent activity as antagonists of the CCL1-CCR8 signaling pathway, even under experimental conditions mimicking the acidic tumor microenvironment, as also demonstrated in Example 14. Accordingly, the monoclonal antibody (or antigen-binding fragment thereof) according to the invention, which specifically binds to human CCR8 and is an antagonist, particularly an insurmountable antagonist, of the CCL1-CCR8 signaling pathway, preferably has an antagonistic activity (particularly an insurmountable antagonistic activity) on the CCL1-CCR8 signaling pathway at a pH within the range from 6.2 to 6.9, more preferably at a pH of 6.5. Moreover, the antibody or antigen-binding fragment according to the invention preferably has an antagonistic activity (particularly an insurmountable antagonistic activity) on CCL1-induced CCR8-Gi2 signaling at a pH within the range from 6.2 to 6.9, more preferably at a pH of 6.5.

A number of corresponding exemplary antibodies are provided herein, which have been characterized in terms of their amino acid sequence. In line with this, the present invention particularly relates to a monoclonal antibody or an antigen-binding fragment thereof, which specifically binds to human CCR8, and which has the CDRs and/or which has the heavy chain variable domain (VH) and the light chain variable domain (VL) as described in any of the embodiments set out in the present specification. The invention further provides a monoclonal antibody or an antigen-binding fragment thereof, which specifically binds to human CCR8, and which has the heavy-chain and light-chain sequences as set out in any of the embodiments described herein.

The invention also relates to a nucleic acid encoding the heavy chain and/or the light chain of the antibody or antigen-binding fragment provided herein. The nucleic acid may be, e.g., mRNA.

The invention further relates to a vector (which is preferably an expression vector) comprising the nucleic acid according to the invention. Moreover, the invention relates to a host cell comprising the nucleic acid or the vector according to the invention.

The invention furthermore relates to a method of producing the antibody or antigen-binding fragment according to the invention, the method comprising culturing the host cell provided herein and isolating the antibody or antigen-binding fragment. The host cell may be, in particular, a CHO cell (e.g., a CHO-K1 cell). The invention also relates to an antibody or antigen-binding fragment (as described herein above), which is obtainable (or obtained) by this method.

The present invention further relates to a composition (which is preferably a pharmaceutical composition) comprising the antibody or antigen-binding fragment according to the invention or the nucleic acid according to the invention.

The invention likewise provides a lipid particle comprising one or more nucleic acids according to the invention (which nucleic acids may be, e.g., mRNA).

The present invention further relates to an antibody or antigen-binding fragment (as described herein) for use in therapy (or for use as a medicament), particularly for use in the treatment of cancer. The invention likewise relates to a nucleic acid (as described herein), a composition (as described herein), or a lipid particle (as described herein), for use in therapy (or for use as a medicament), particularly for use in the treatment of cancer.

Moreover, the invention relates to the use of an antibody or antigen-binding fragment (as described herein) for the manufacture of a medicament for the treatment of cancer. The invention also relates to the use of a nucleic acid (as described herein), a composition (as described herein), or a lipid particle (as described herein) for the manufacture of a medicament for the treatment of cancer.

The invention further provides a method of treating cancer in a subject in need thereof, the method comprising administering to the subject a therapeutically effective amount of an antibody or antigen-binding fragment (as described herein). The subject to be treated may be, in particular, a human being. The invention likewise relates to a method of treating cancer in a subject in need thereof, the method comprising administering to the subject a therapeutically effective amount of (i) a nucleic acid (as described herein), (ii) a composition (as described herein), or (iii) a lipid particle (as described herein). The subject to be treated in any of these methods may be, in particular, a human being.

The present invention is illustrated by the following figures:

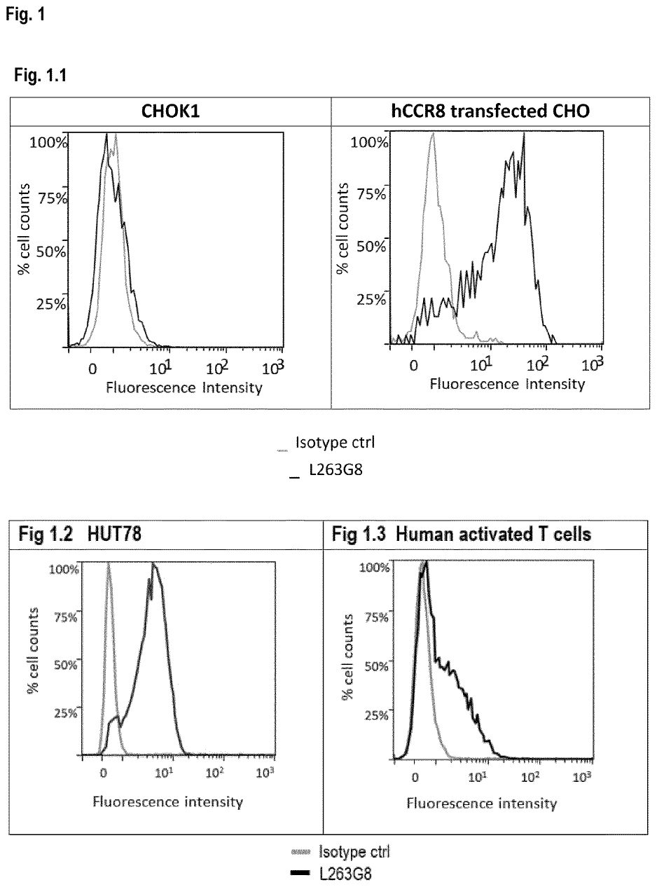

FIG. 1 shows the cellular reactivity of the reference anti-CCR8 mAb L263G8 on hCCR8 transfected CHO cells (FIG. 1.1), HUT78 cells (FIG. 1.2) and human activated T cells (FIG. 1.3) by flow cytometry. Representative experiment. See Examples 7, 8 and 9.

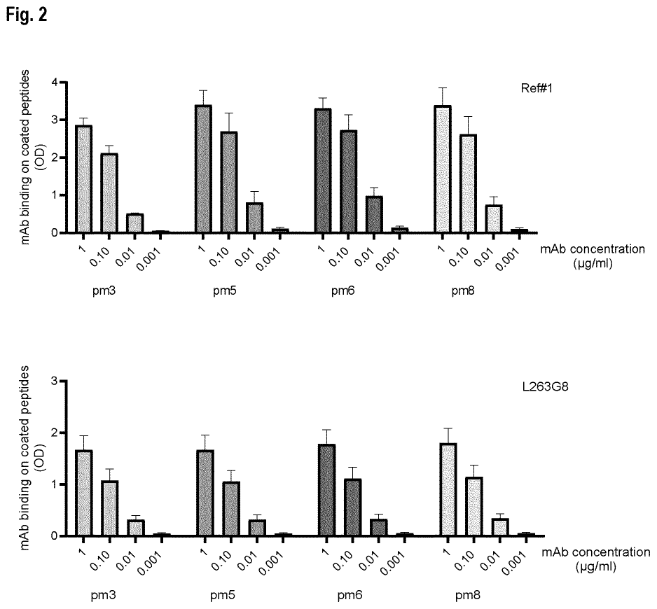

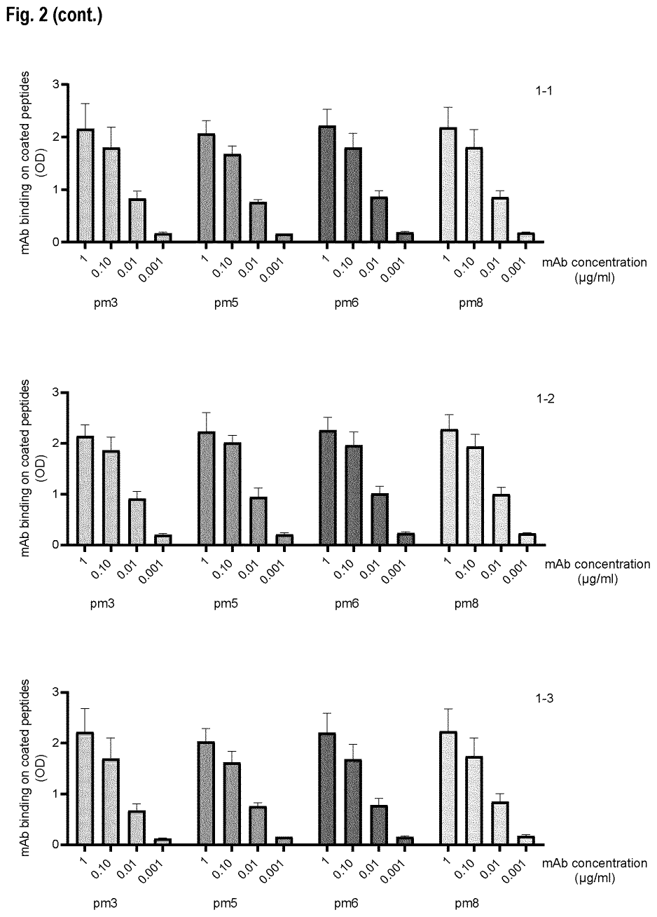

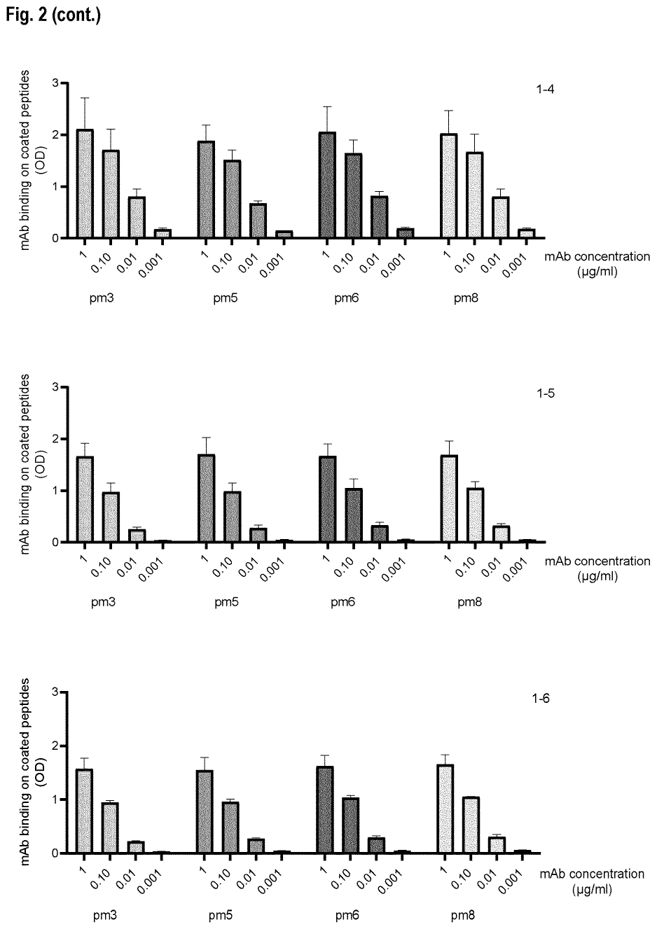

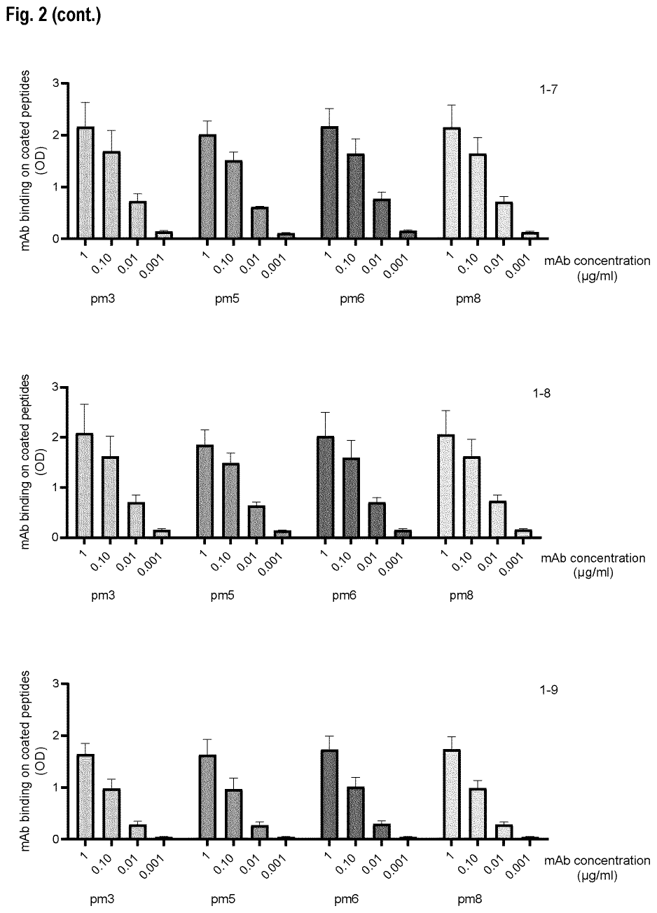

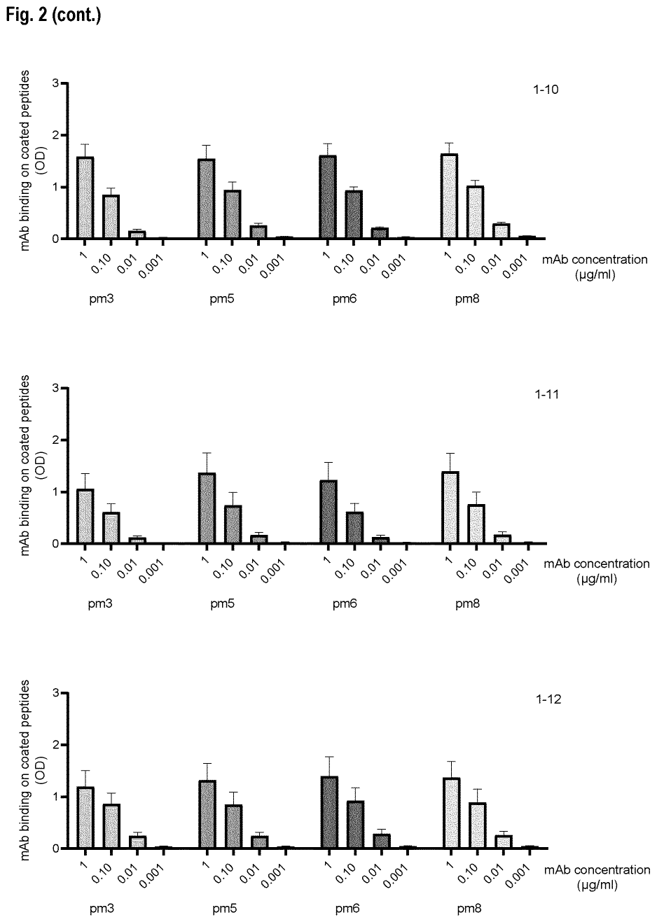

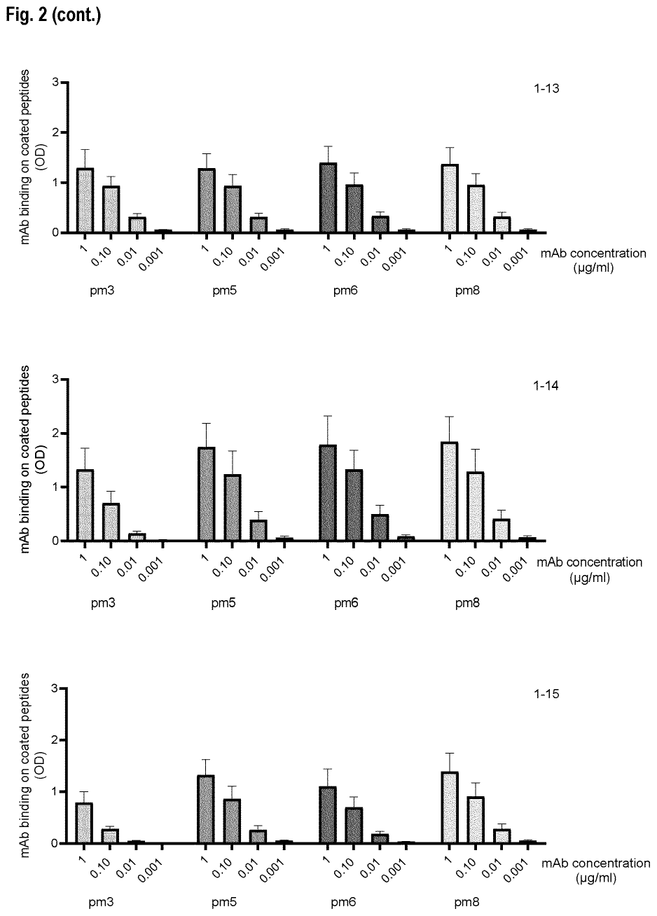

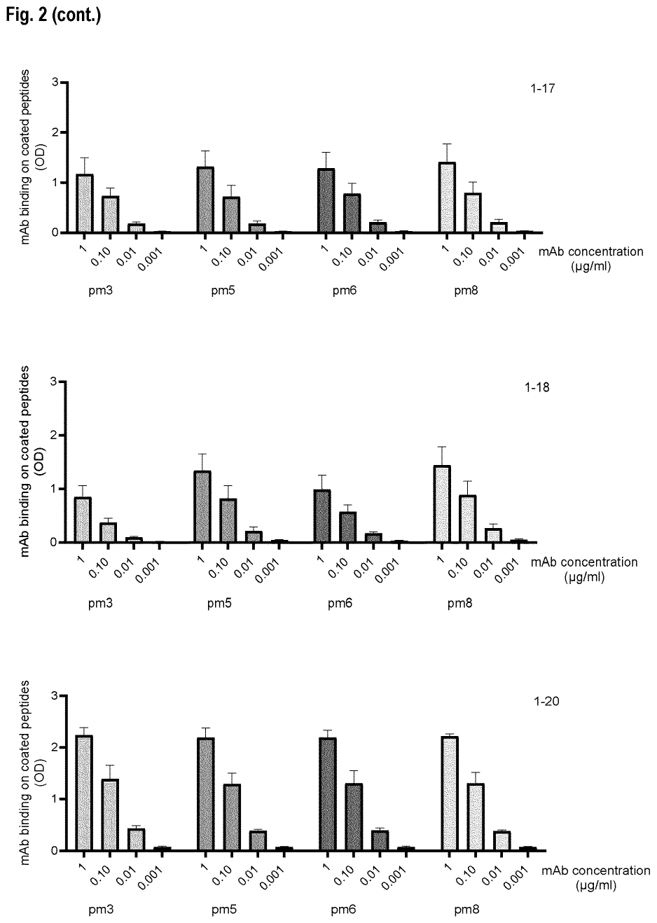

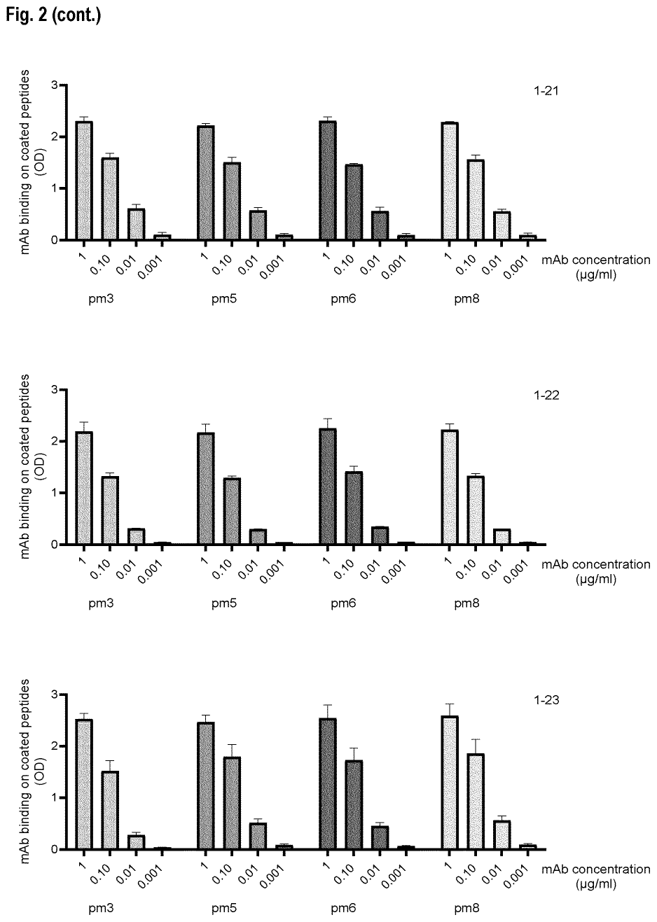

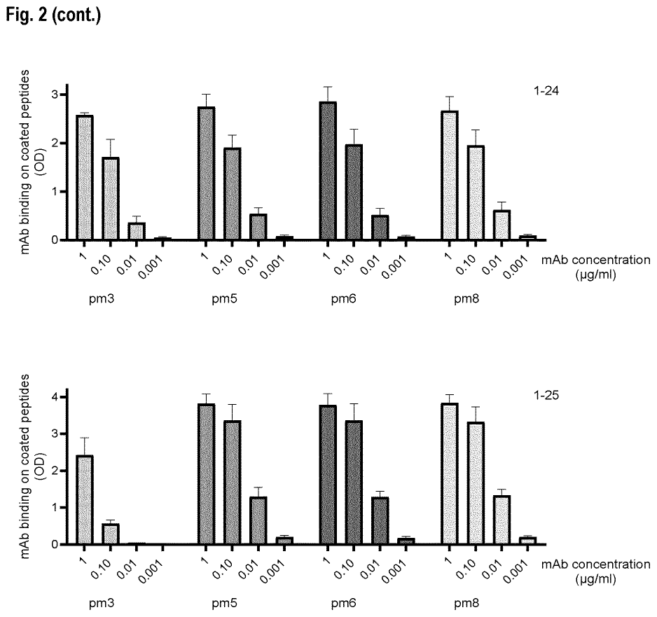

FIG. 2 shows the mAb reactivity on the CCR8 related peptides pm3, pm5, pm6 and pm8 analyzed by ELISA. Mean+/−SD on two independent experiments. See Example 10.

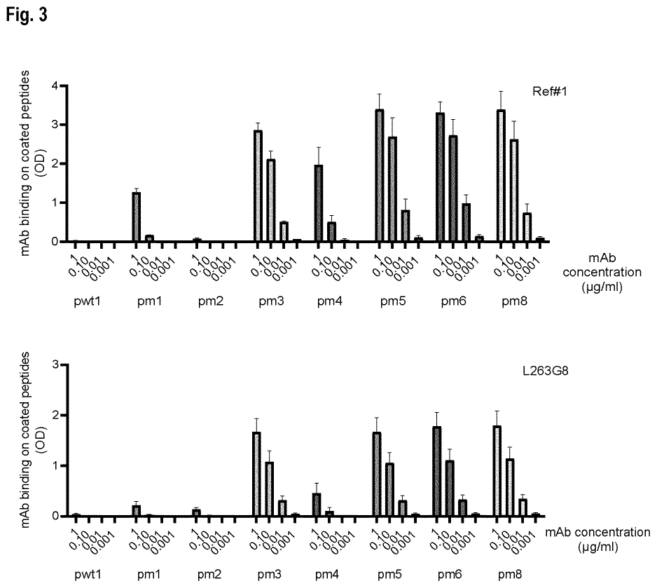

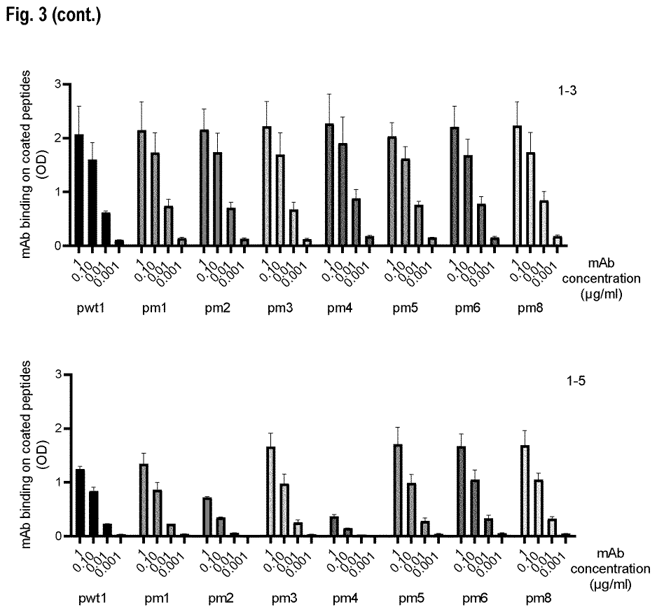

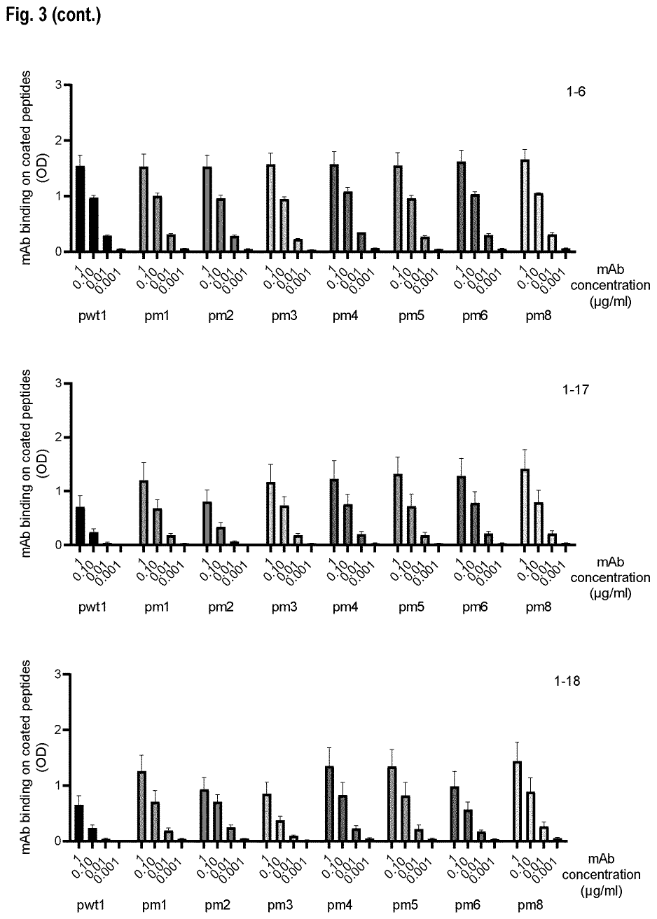

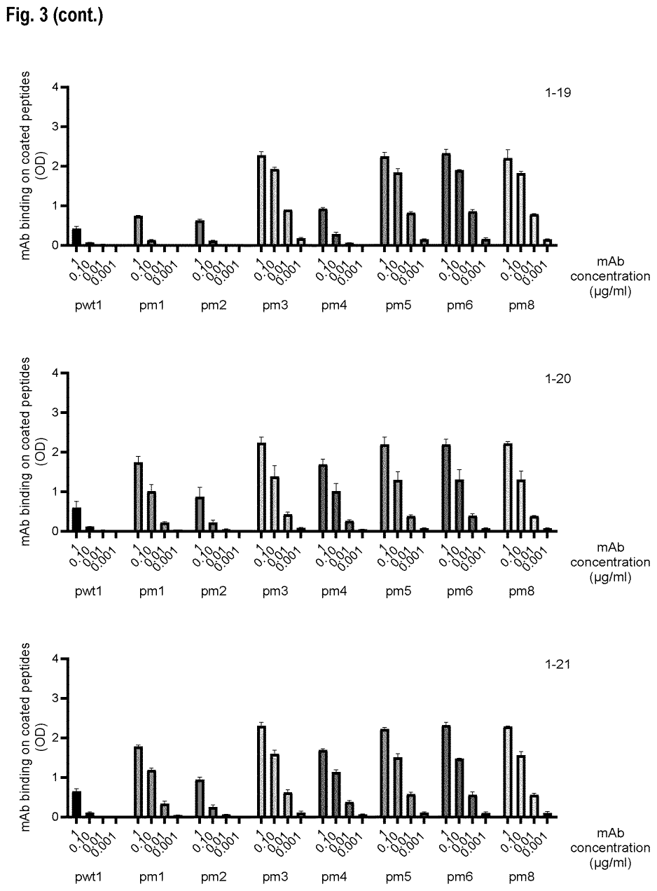

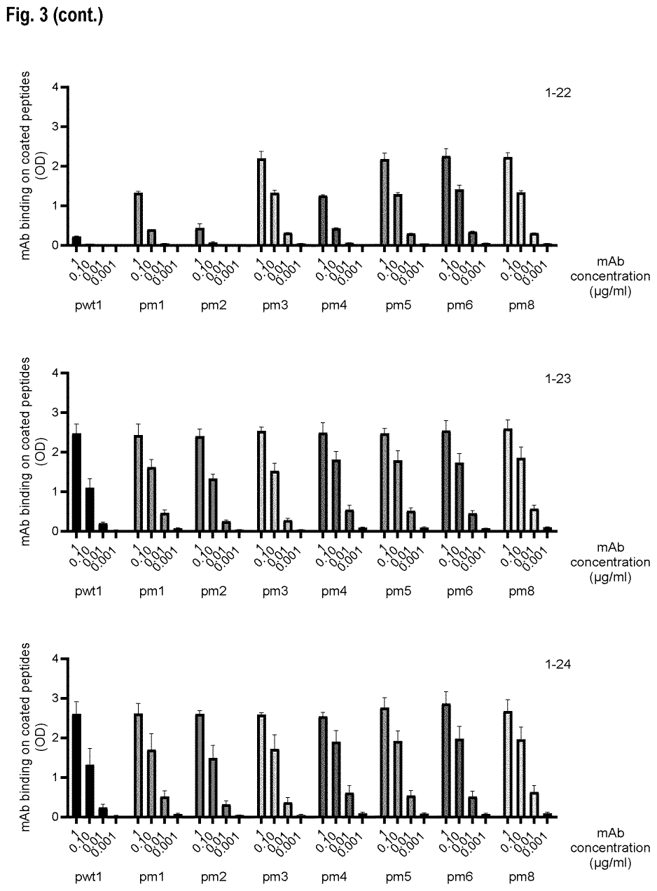

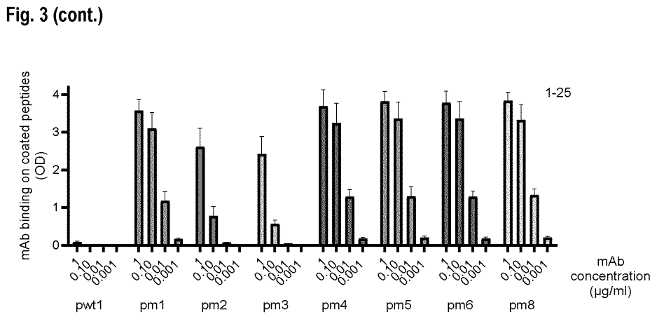

FIG. 3 shows the mAb reactivity on an extended panel of CCR8 related peptides analyzed by ELISA. Mean+/−SD on two independent experiments. See Example 10.

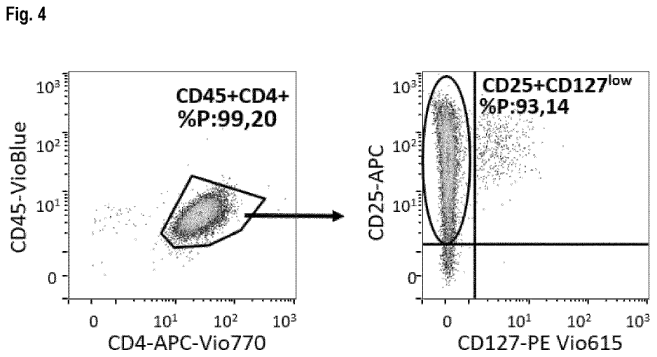

FIG. 4 shows the immunophenotyping of human activated T cells composed of at least 80% of CD45+CD4+CD25+CD127^{low} cells. Representative experiment. See Example 11.

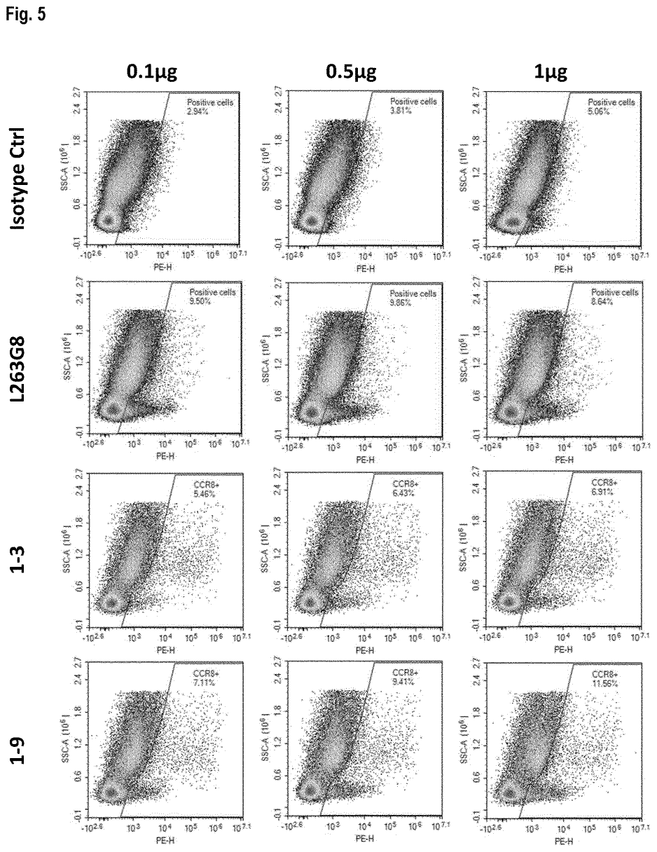

FIG. 5 illustrates the mAb reactivity on lymphocytes from ascitic ovarian cancers by flow cytometry. Representative experiment. See Example 11.

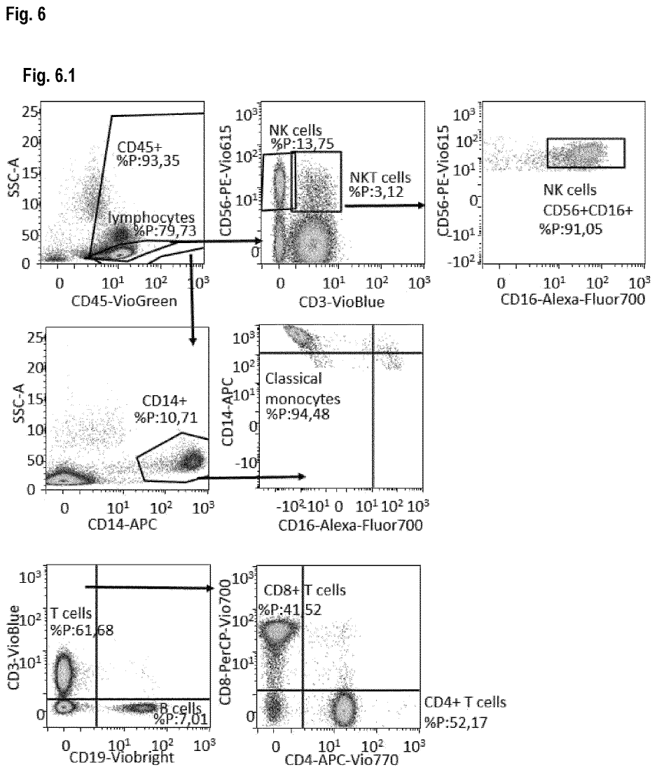

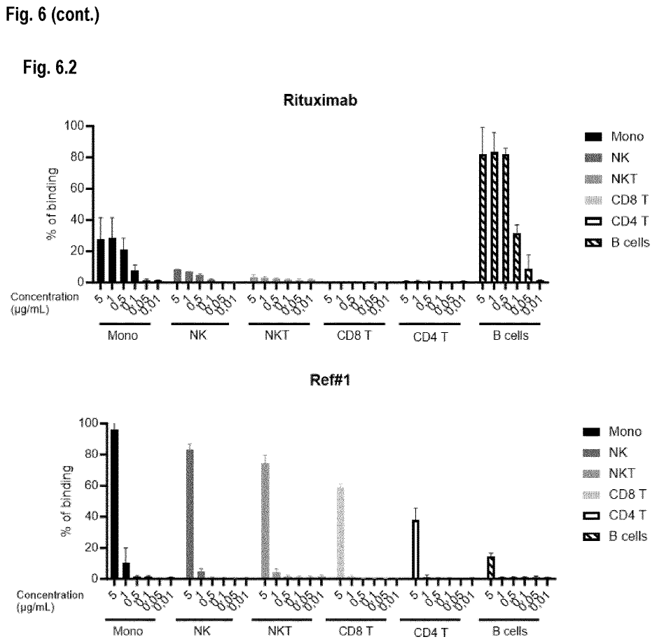

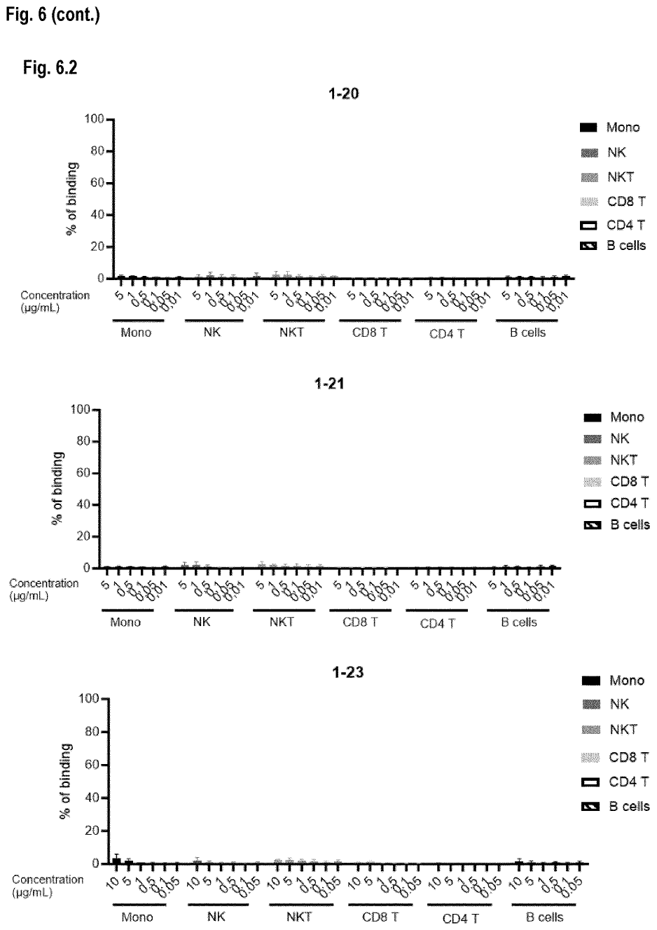

FIG. 6 shows mAb reactivity on Peripheral Blood Mononuclear Cells (PBMC) from healthy donors. FIG. 6.1 presents the gating strategy allowing the discrimination of the different immune cells within PBMC. FIG. 6.2 shows anti-CCR8 mAb binding on PBMC. Mean of percentage of binding on different immune cells from two independent donors. See Example 12.

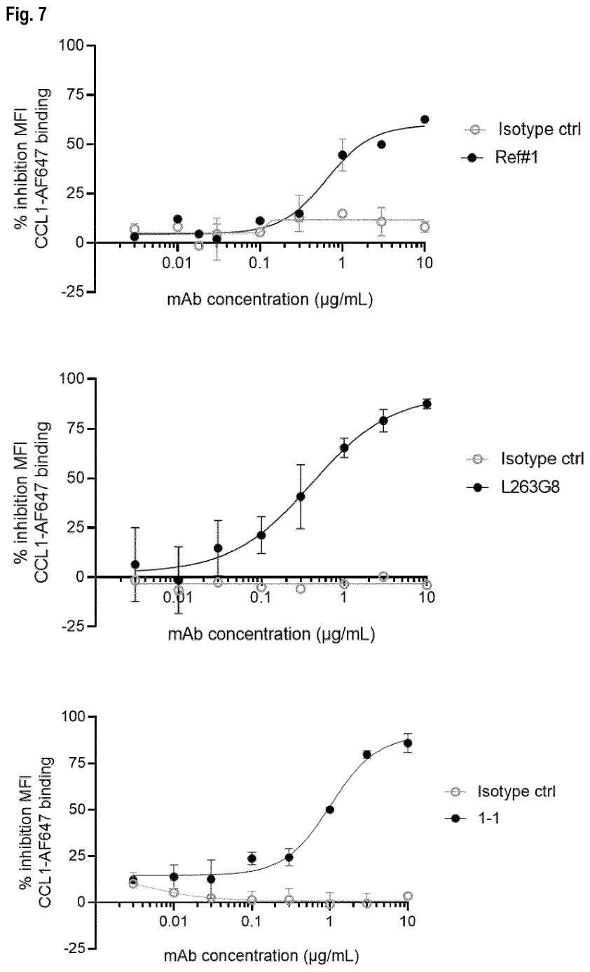

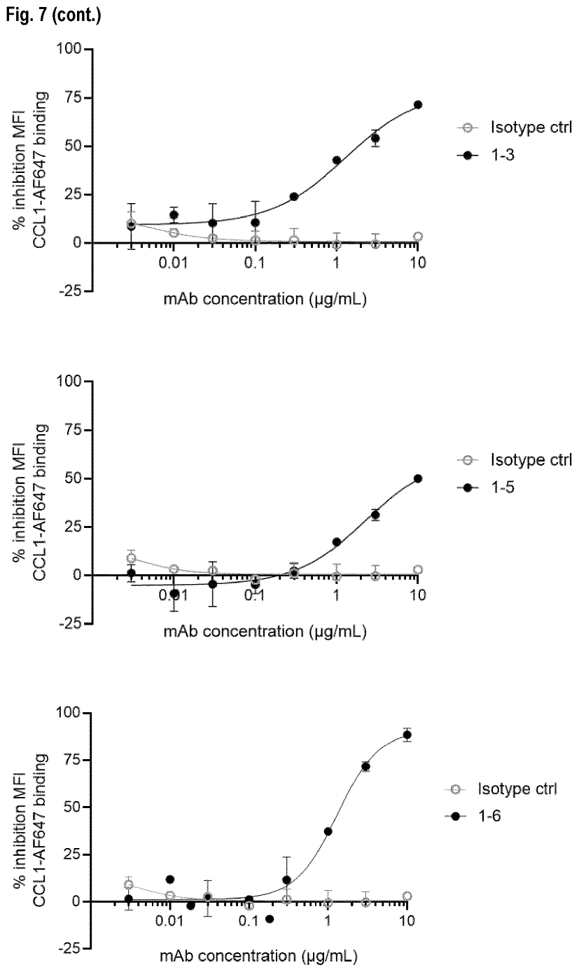

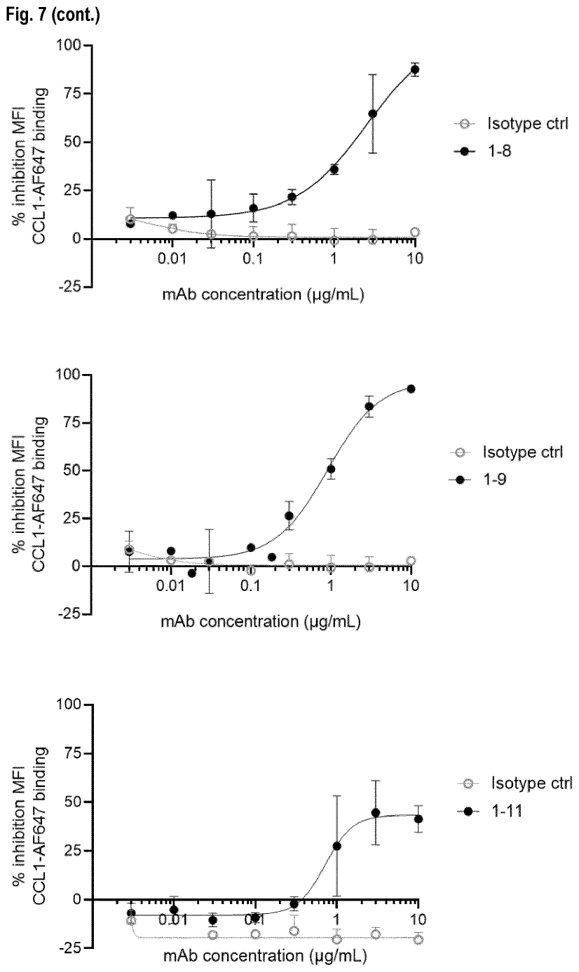

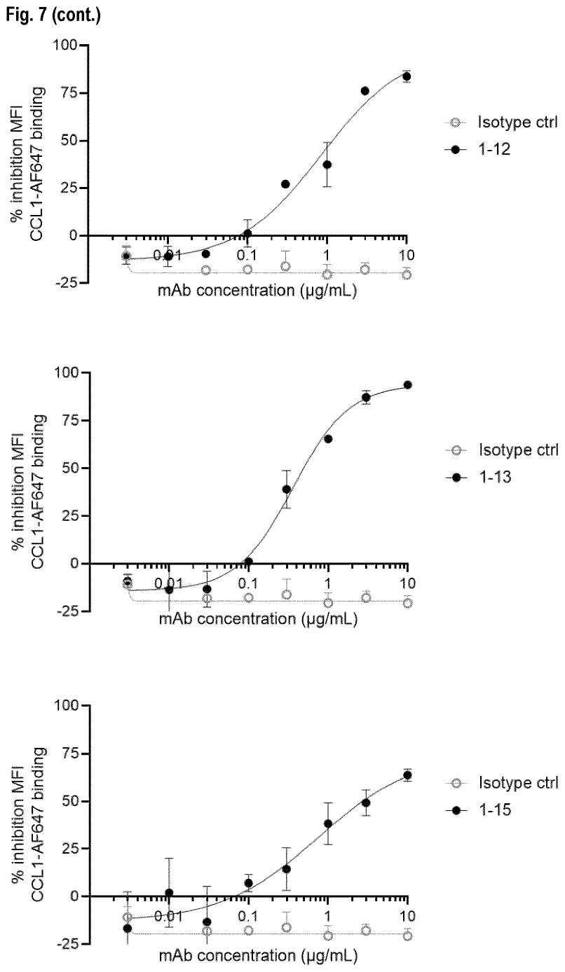

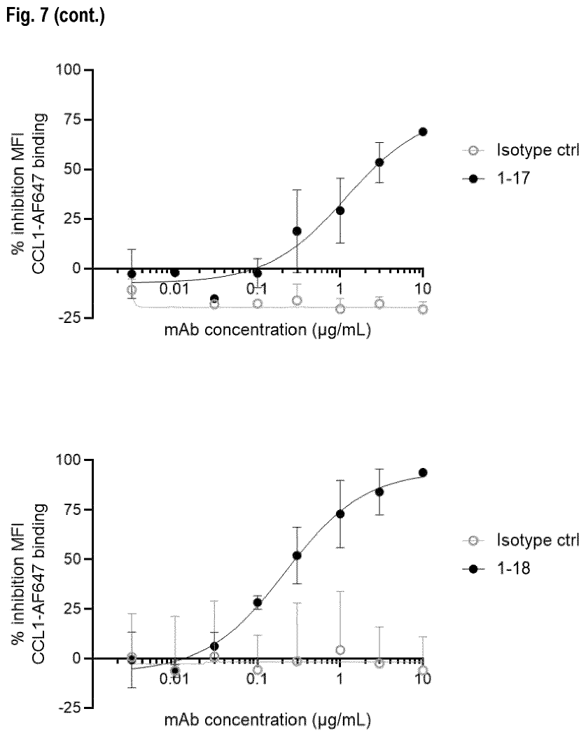

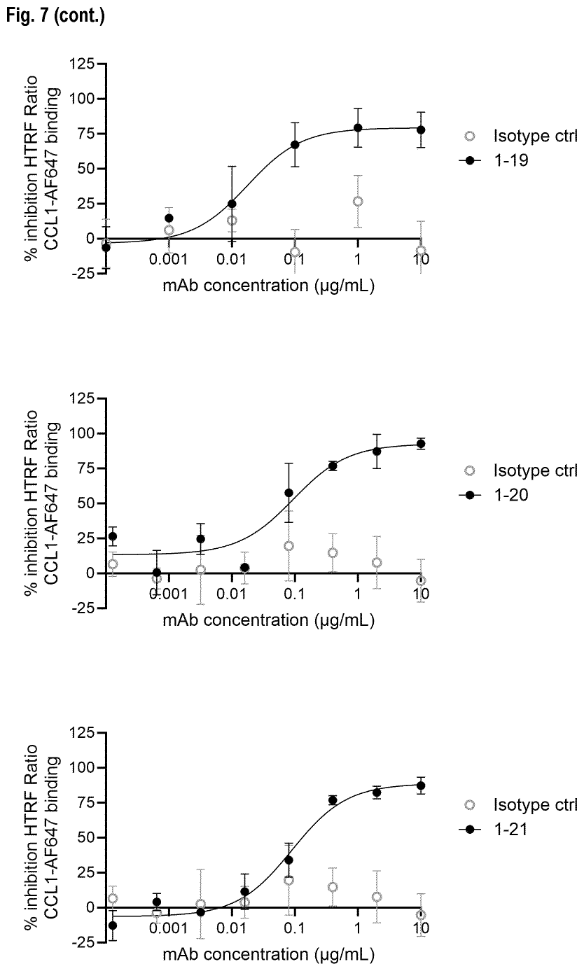

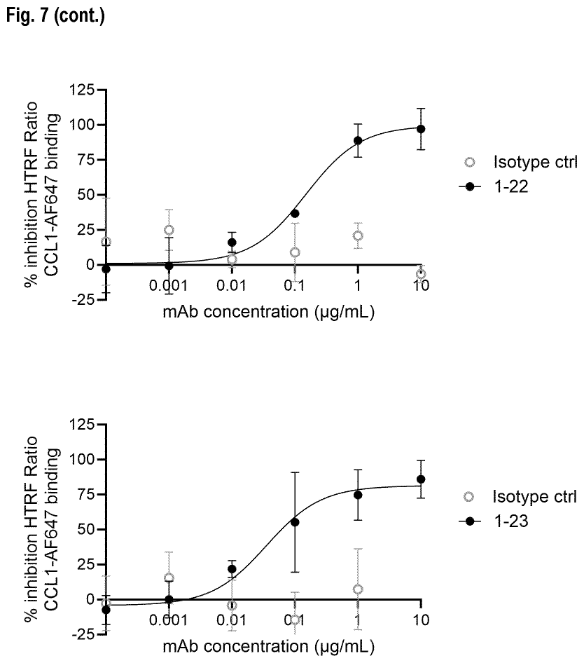

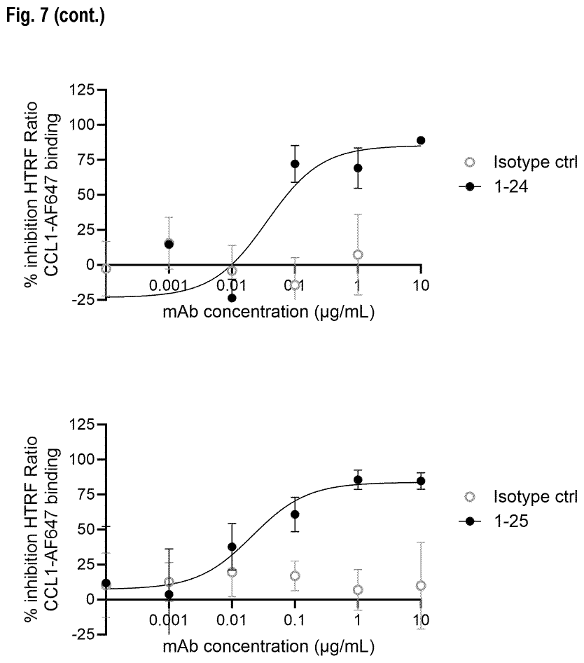

FIG. 7 shows the mAb impact on CCL1 binding on hCCR8 analyzed by flow cytometry and/or HTRF on hCCR8 transfected CHO cell line and HEK-293 cell line, respectively. Mean of two independent experiments. See Examples 13.1 and 13.2.

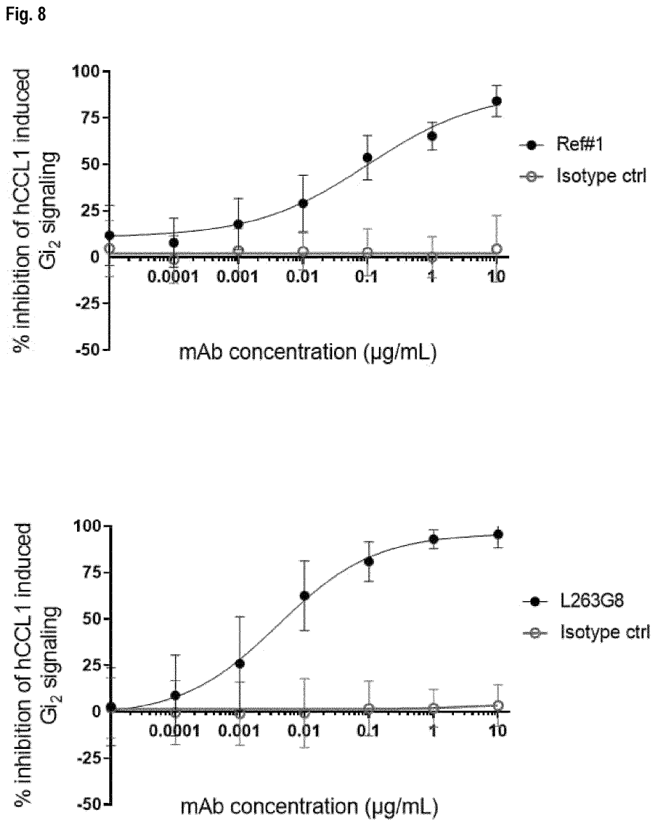

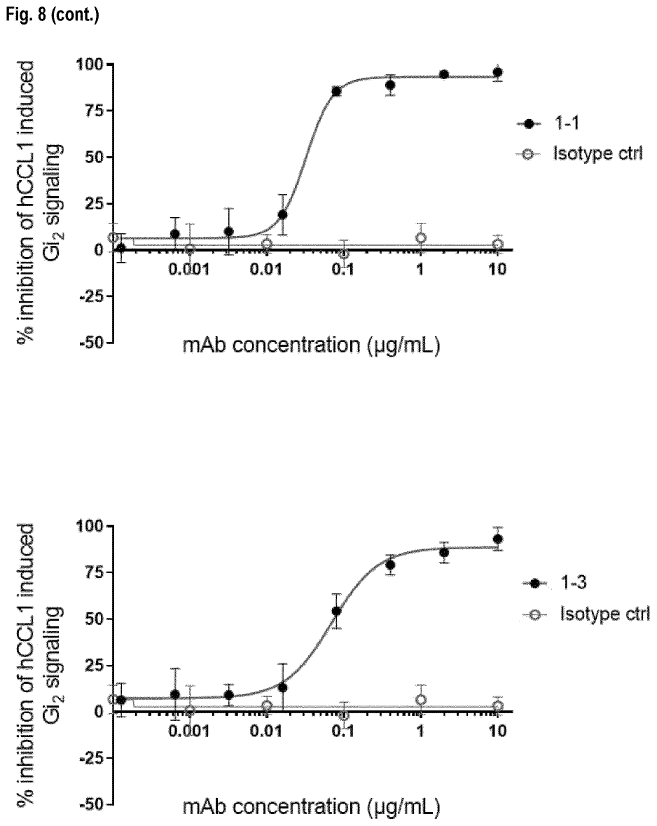

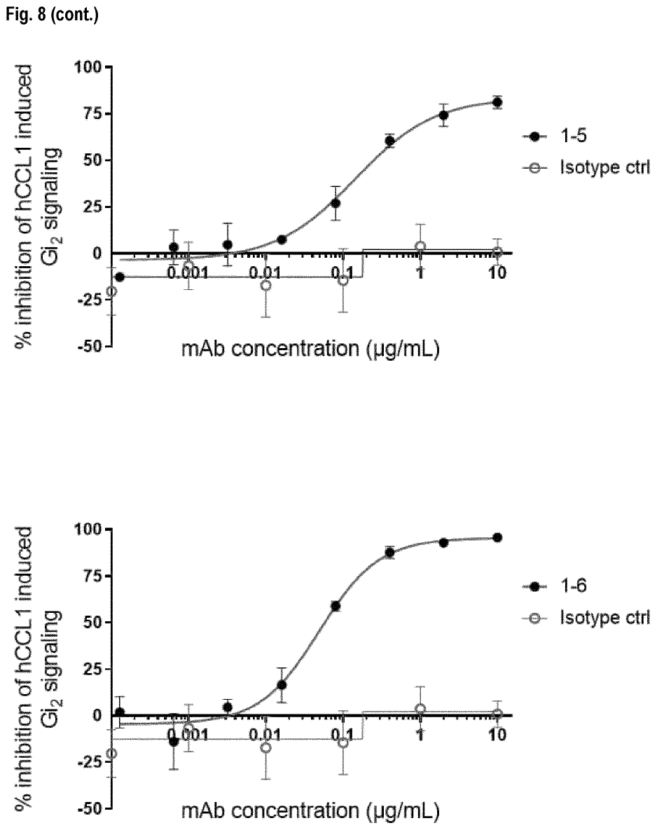

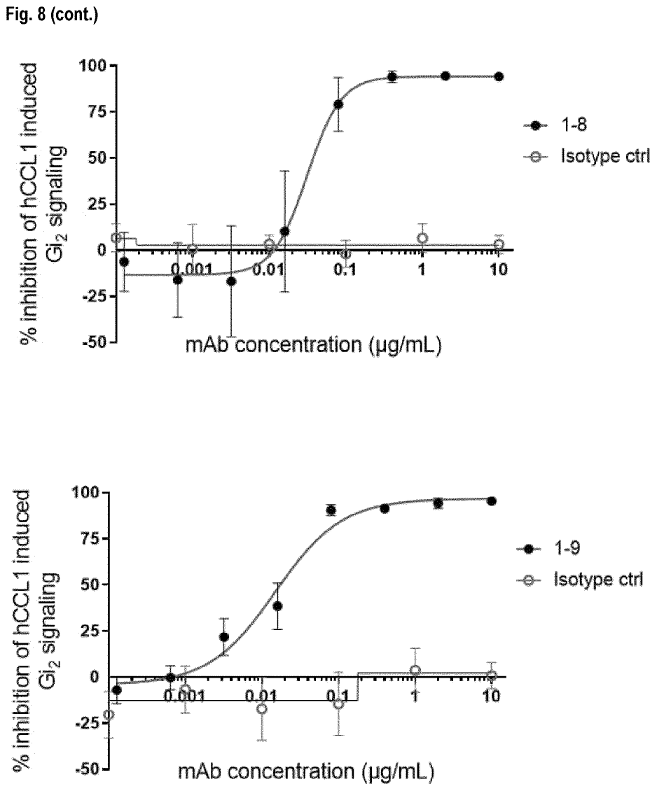

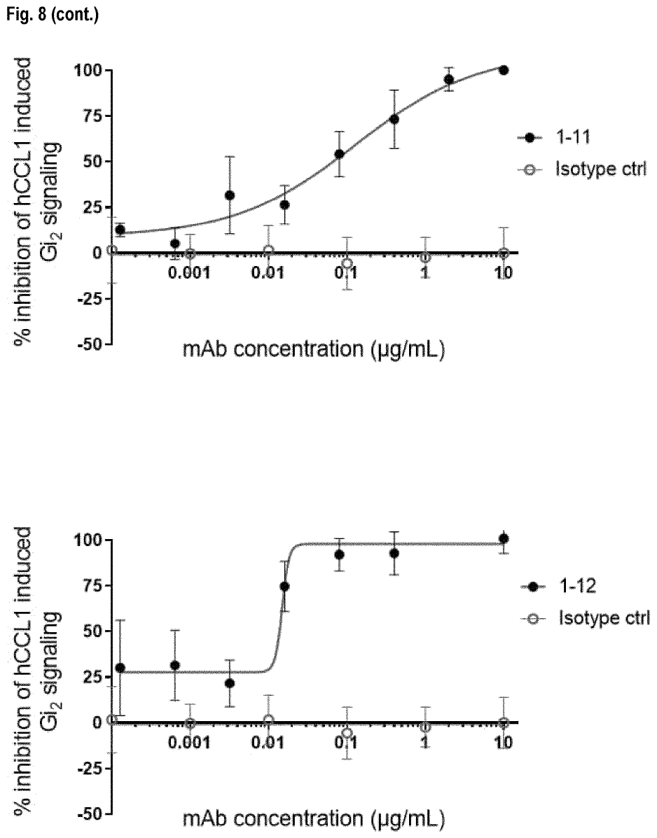

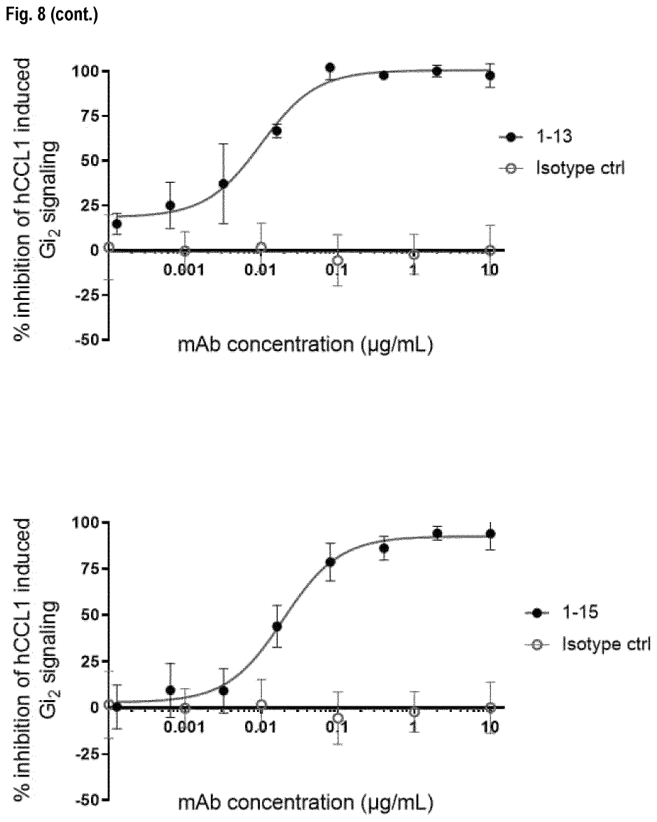

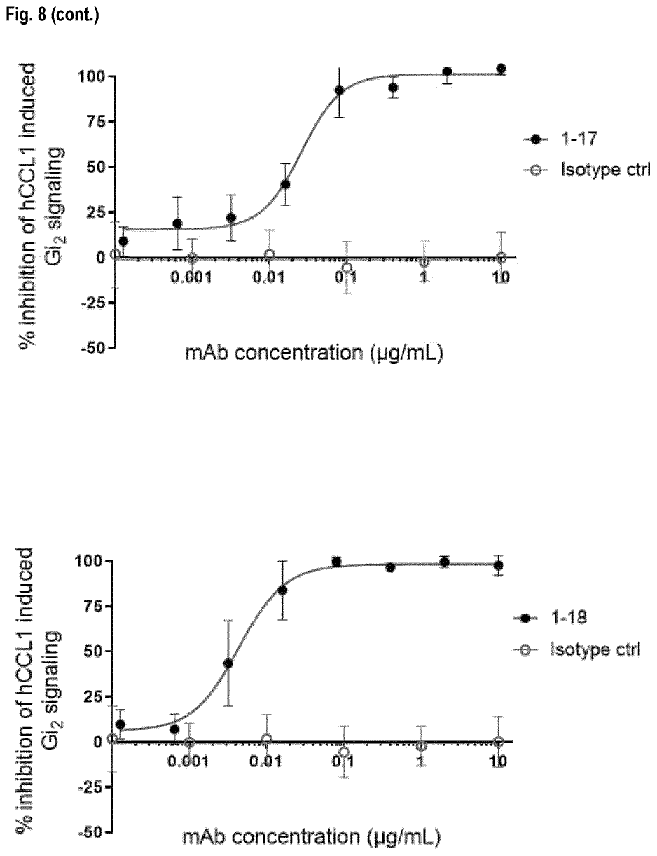

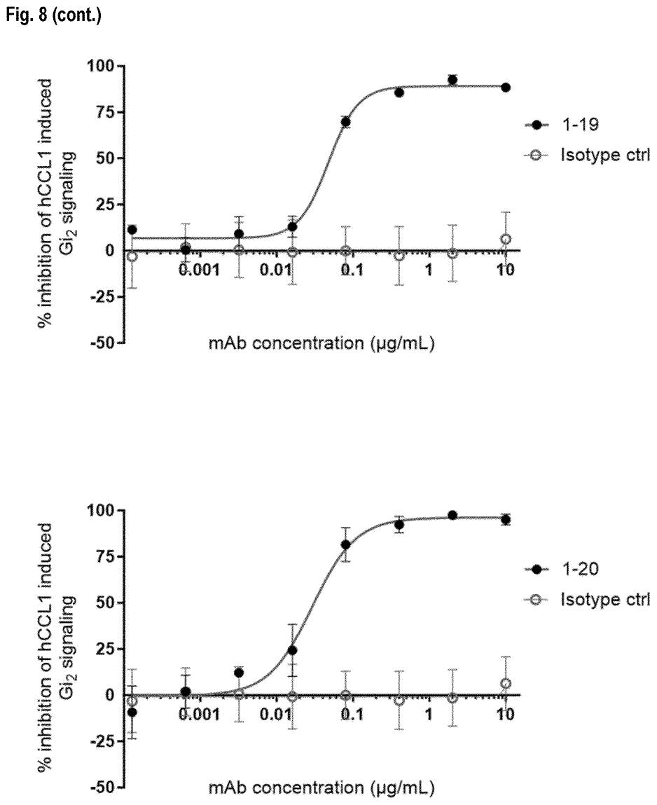

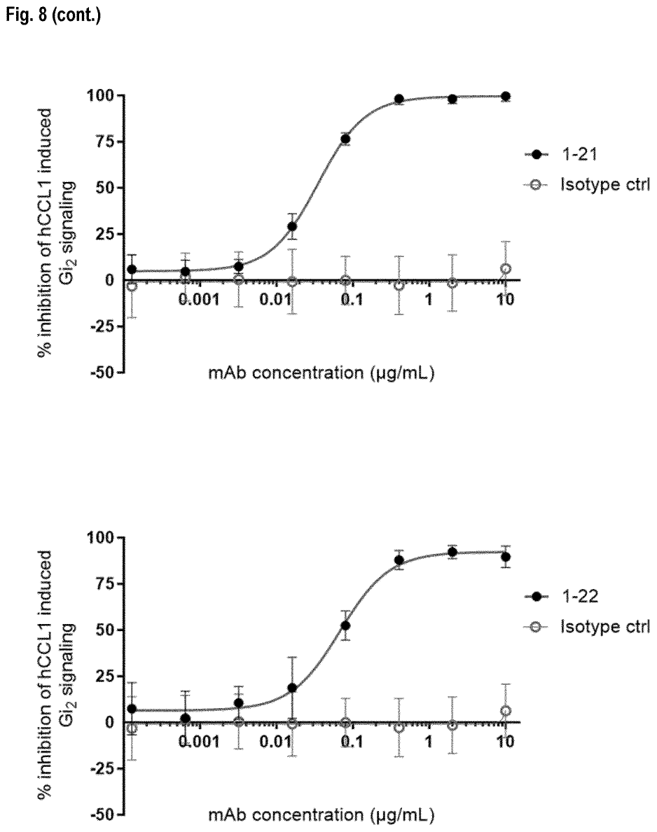

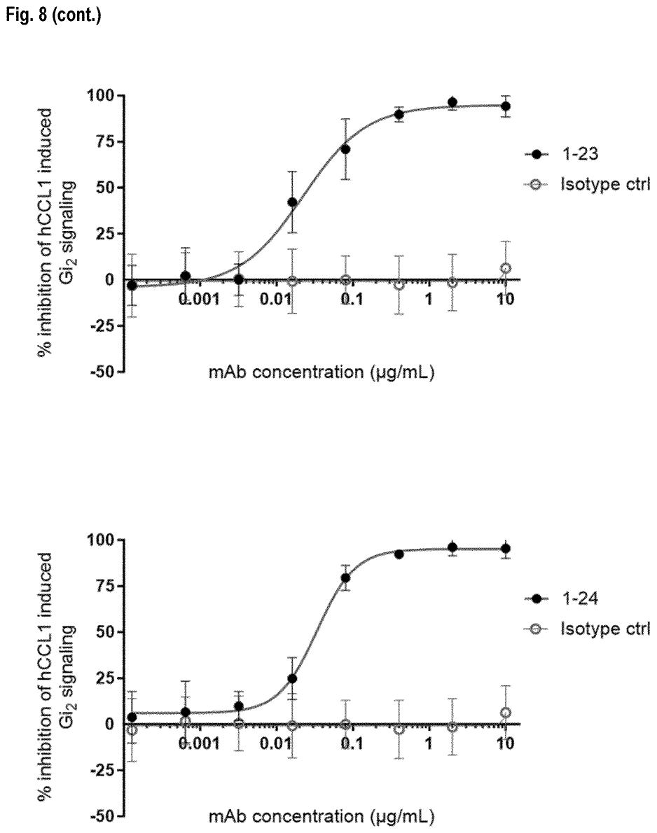

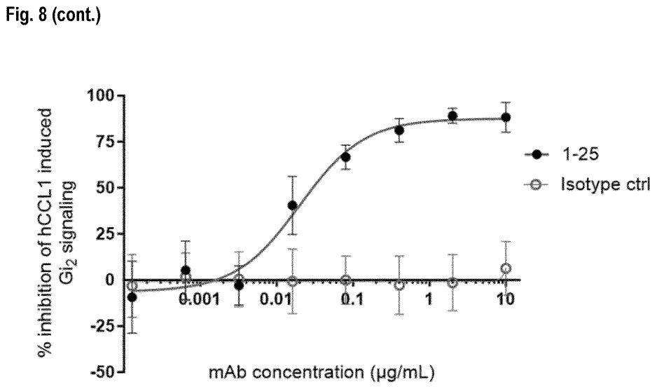

FIG. 8 shows the mAb impact on CCL1 induced hCCR8 Gi2 signaling pathway analyzed by BRET on transiently transfected HEK 293 cell line. Mean+/−SD on two independent experiments. See Example 14.

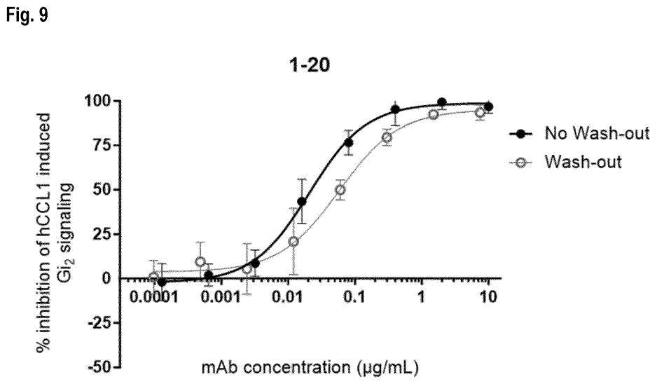

FIG. 9 shows the action time of mAb on CCL1 induced hCCR8 Gi2 signaling pathway in transiently transfected HEK 293 cell line. The assay is done with and without a wash-out step to remove the excess of unbound antibodies in the solution. Six hours post mAb wash-out, mAb maintained their antagonist activity on hCCR8, indicating a long action time on CCR8. Mean+/−SD on two independent experiments. See Example 14.

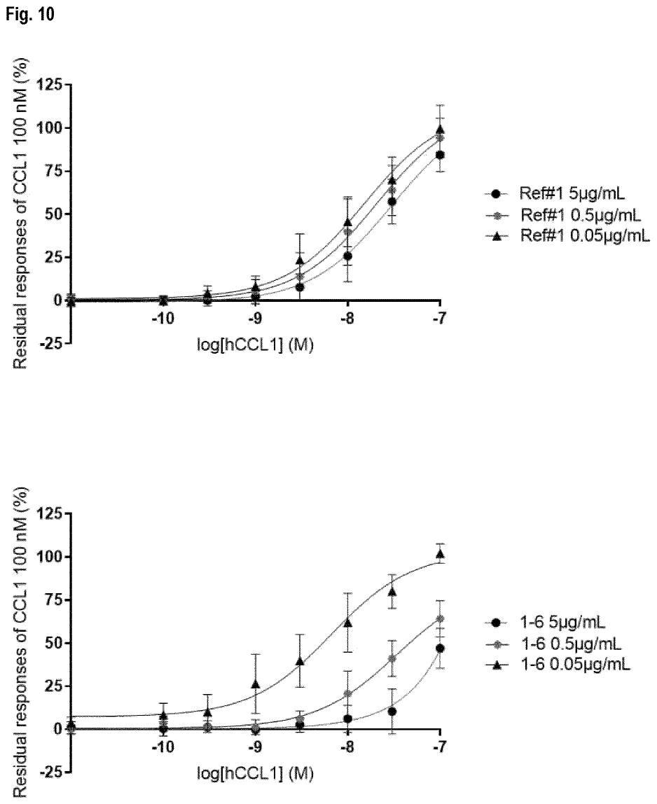

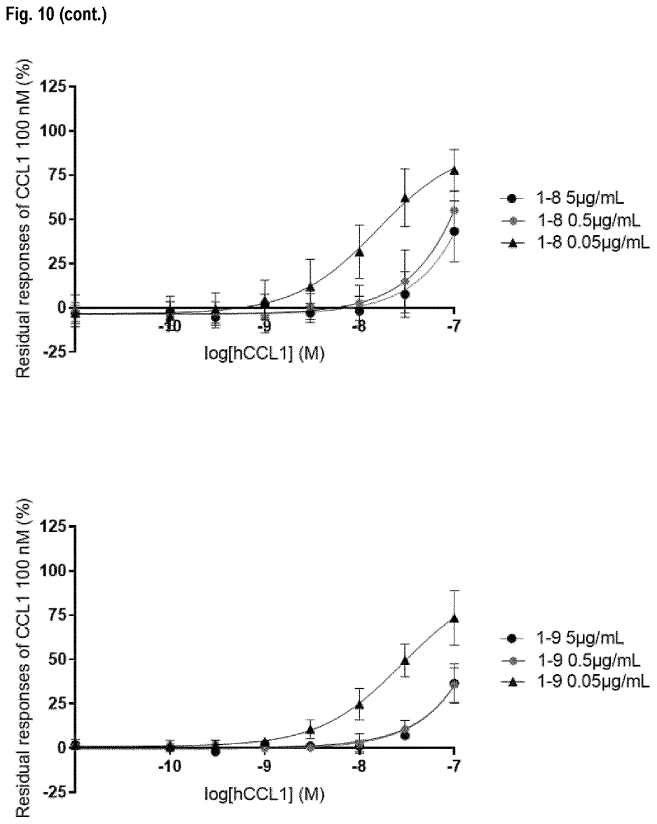

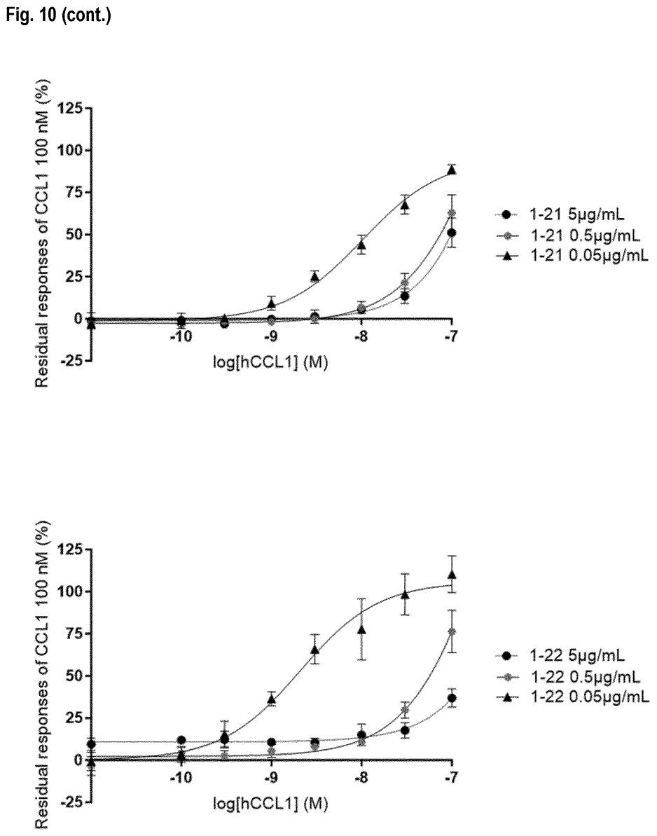

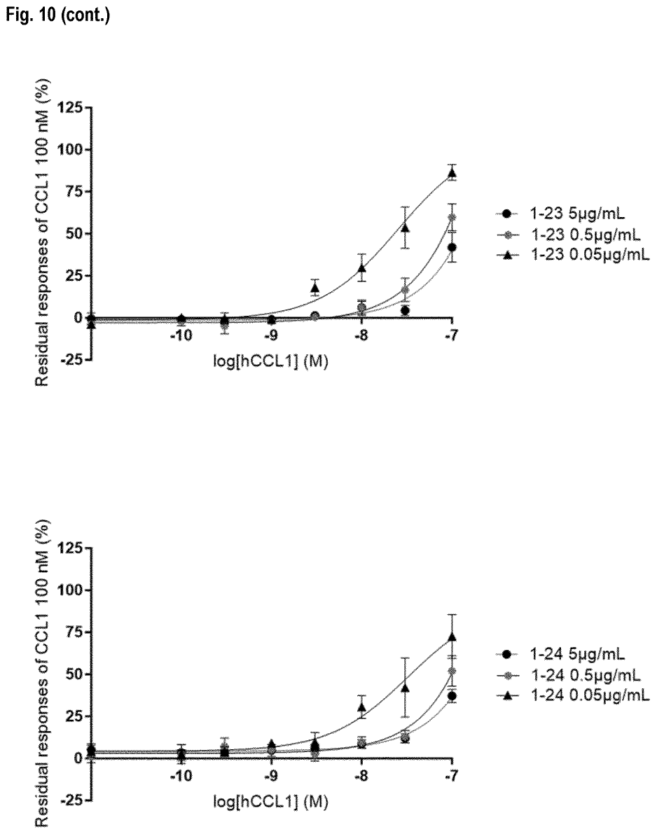

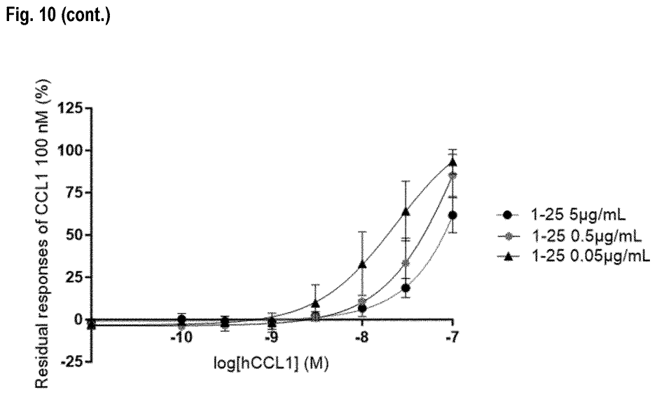

FIG. 10 shows the mAb characterization related to insurmountable antagonist effect on CCL1 induced Gi2 signaling pathway analyzed by BRET on transiently transfected HEK 293 cell line. Representative experiment performed with duplicates. See Example 14.

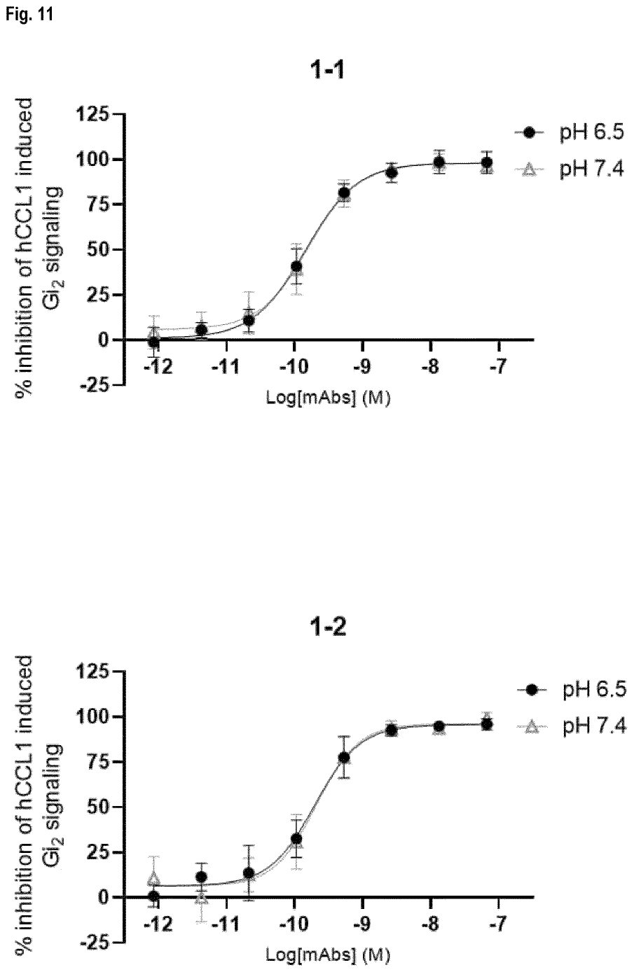

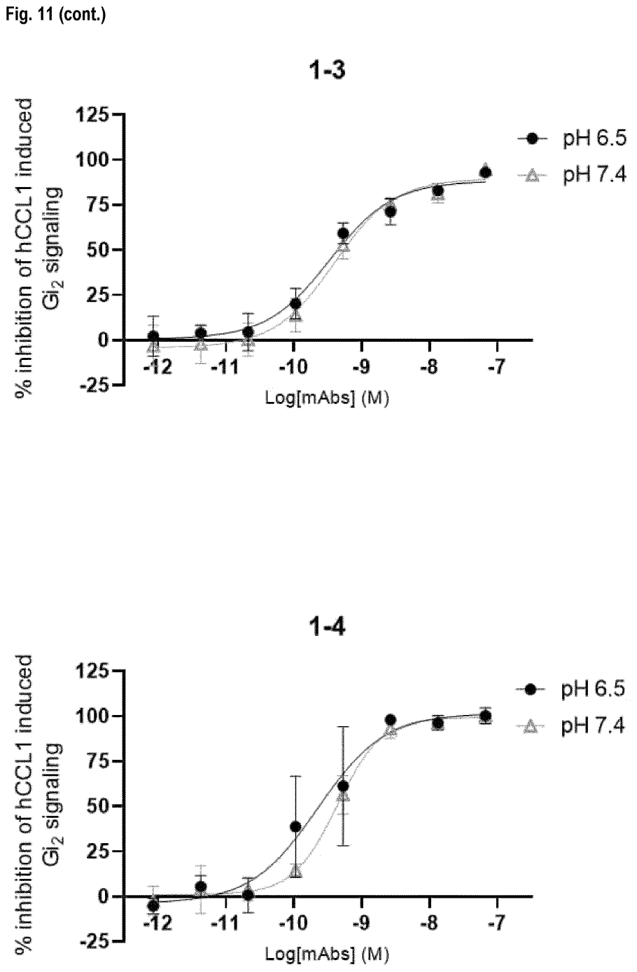

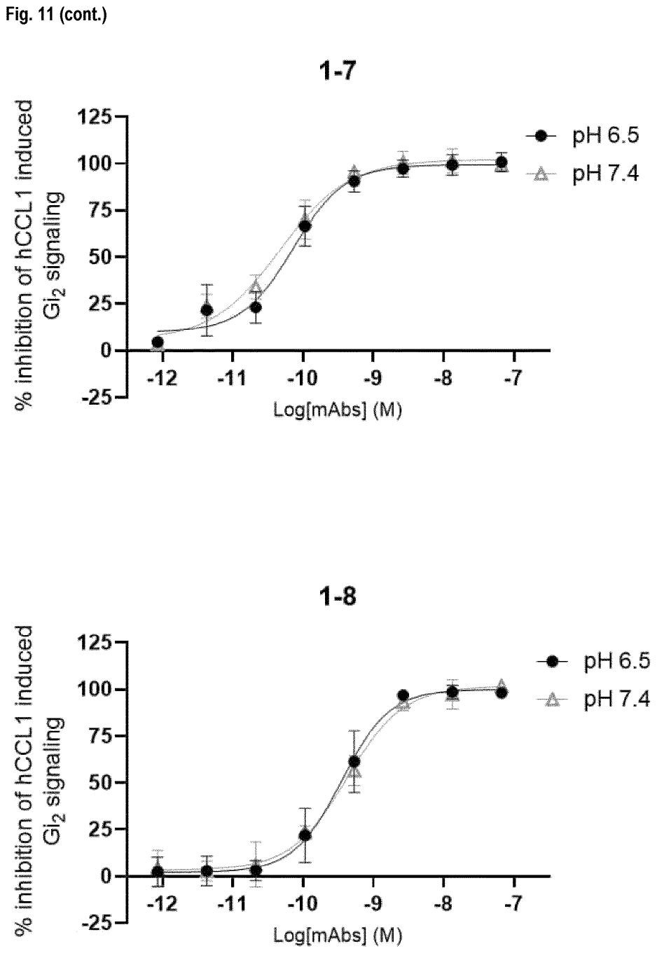

FIG. 11 shows the mAb impact on CCL1 induced hCCR8 Gi2 signaling pathway analyzed by BRET on transiently transfected HEK 293 cell line at pH 6.5 or pH 7.4. Mean+/−SD on two independent experiments. See Example 14.

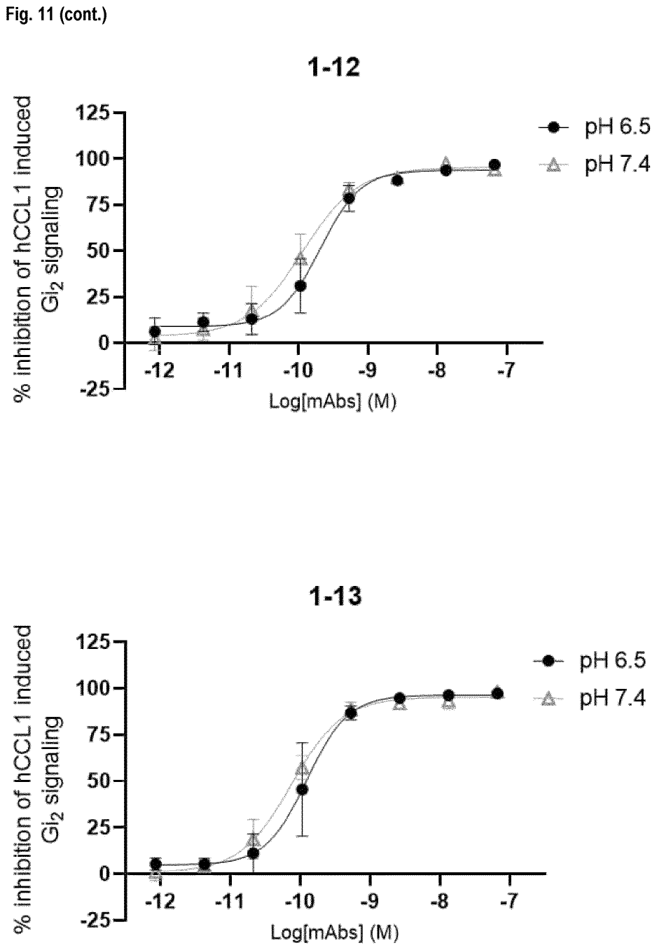

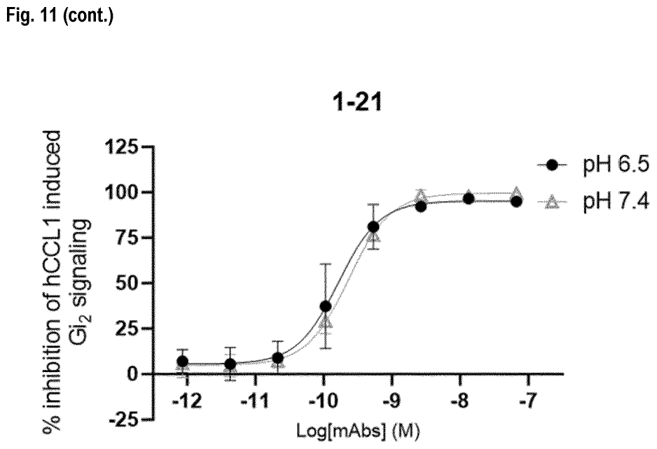

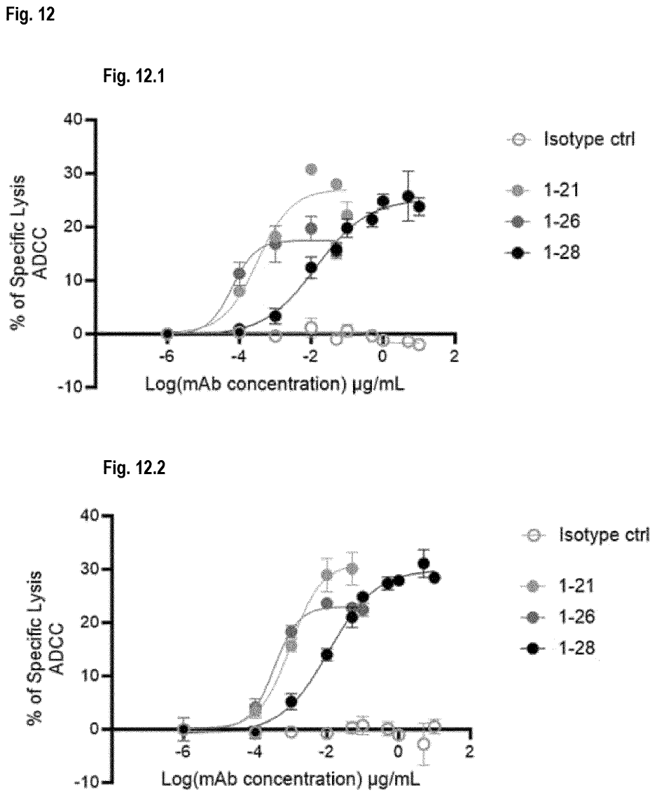

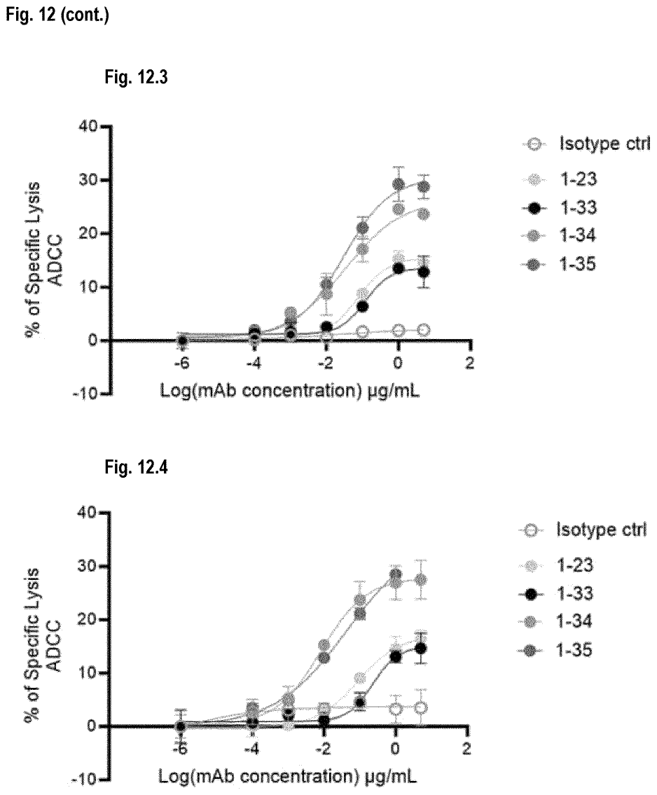

FIG. 12 shows the antibody-dependent cellular cytotoxicity (ADCC) effect of the antibodies according to the invention on CCR8 expressing HUT78 cell line. FIGS. 12.1 to 12.4 show dose response curves of percentage of specific lysis ADCC induced by anti-CCR8 mAbs. See Example 16.

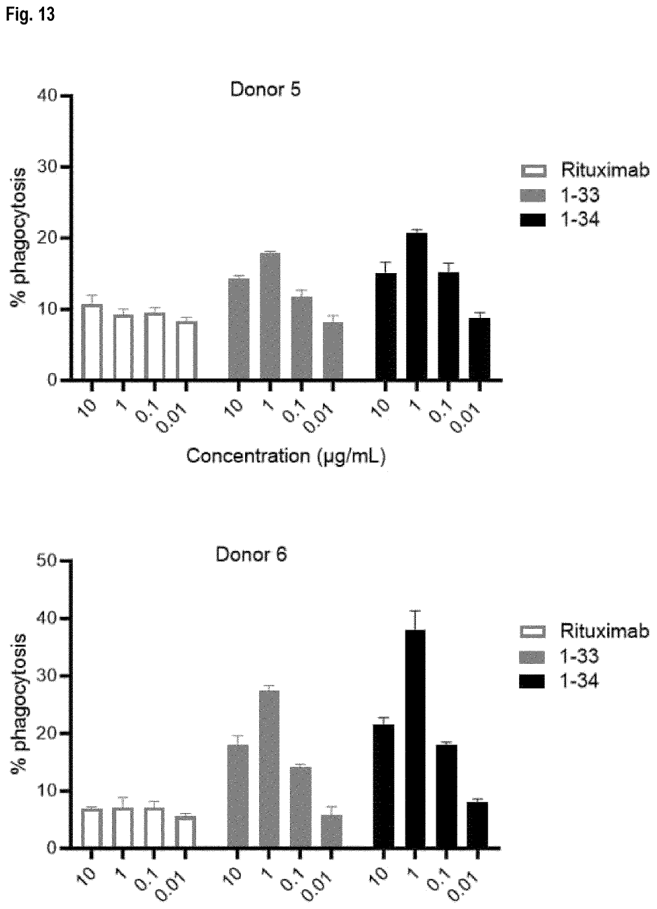

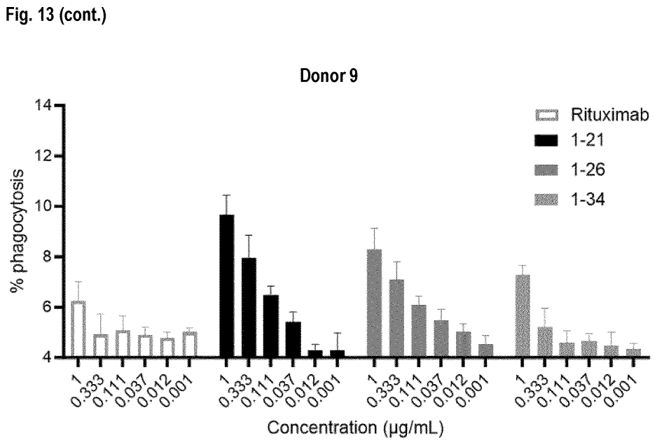

FIG. 13 shows the antibody-dependent cellular phagocytosis (ADCP) activities of anti-CCR8 antibodies on HUT78 as target cells and Monocyte-Derived Macrophages as effector cells. Percentage of phagocytosis of three independent donors. See Example 17.

As described above, the present invention relates to a monoclonal antibody or an antigen-binding fragment thereof, wherein said antibody or antigen-binding fragment specifically binds to human CCR8 and is an antagonist of the CCL1-CCR8 signaling pathway; preferably wherein the antibody or antigen-binding fragment is an insurmountable antagonist of the CCL1-CCR8 signaling pathway.

For the sake of brevity, the monoclonal antibodies (or antigen-binding fragments thereof) provided in accordance with the present invention, which specifically bind to human CCR8, are also interchangeably referred to herein as “anti-CCR8 antibodies” or “anti-CCR8 mAbs”.

Antibodies are well-known in the art and are also referred to as immunoglobulin molecules. In general, immunoglobulin molecules are capable of specifically binding to a target (such as, in the present case, human CCR8) via at least one antigen recognition site, which is typically located in the variable region of the immunoglobulin molecule. The term “antibody”, as used herein, encompasses not only intact (e.g., full-length) antibodies, particularly monoclonal antibodies, but also antigen-binding fragments thereof as well as any modified antibodies, antibody constructs, and fusion proteins (or other molecules) comprising one or more antigen-binding antibody portions or fragments. Non-limiting examples of antigen-binding antibody fragments, as known in the art, include Fab, Fab′, F(ab′)₂, or Fv, a single-chain antibody (e.g., a single-chain variable fragment (scFv)), a heavy-chain antibody, a single-domain antibody (e.g., a nanobody, a single VH domain antibody (or V_{H}H fragment), or an IgNAR single-domain antibody (or V_{NAR} fragment)), a multi-specific antibody (e.g., a bispecific antibody), or a diabody. Moreover, any such antibodies may be, e.g., murine antibodies, human (or “fully human”) antibodies, humanized antibodies, or chimeric antibodies. An antibody may be, in particular, an antibody of a specific class, such as, e.g., IgG, IgM, IgA, IgD, or IgE (or any subclass thereof, such as, e.g., IgG1, IgG2, IgG3, or IgG4; or IgA1 or IgA2). Accordingly, an antibody according to the present invention may be, e.g., an antibody of the IgG class (e.g., IgG1, IgG2, IgG3 or IgG4) which is composed of a light chain and a heavy chain. While the present disclosure includes explicit references to an “antigen-binding fragment” (of an antibody), it will be understood that, unless specifically indicated otherwise or contradicted by context, any reference to an “antibody” includes a specific reference to the corresponding intact (or full-length) antibody as well as a specific reference to an antigen-binding fragment of the corresponding antibody, and preferably refers to the corresponding intact (or full-length) antibody.

The term “monoclonal antibody” is used herein according to its well-known and understood meaning in the art. Monoclonal antibodies can be obtained by different techniques known in the art and, accordingly, are not limited with respect to the method by which they have been obtained. For example, monoclonal antibodies can be made by the hybridoma method (see, e.g., Kohler G et al., Nature, 1975, 256, 495-7; Freysdottir J, Methods Mol Med, 2000, 40:267-79, doi: 10.1385/1-59259-076-4:267; or Hnasko R M et al., Methods Mol Biol, 2015, 1318:15-28, doi: 10.1007/978-1-4939-2742-5_2). Monoclonal antibodies, including fully human as well as humanized antibodies, can also be generated in transgenic animals (particularly transgenic mice), e.g., using commercially available mice that have been engineered to express specific human immunoglobulins. Corresponding transgenic mice include, e.g., XenoMouse®, HuMAb-Mouse®, TransChromo (TC) Mouse™, VelociMouse®, OmniMouse®, Kymouse™, AlivaMab-Mouse, Trianni-Mouse®, or Merus MeMo® Mouse; see also, e.g., Foltz I N et al., Immunol Rev, 2016, 270(1): 51-64, doi: 10.1111/imr.12409; Murphy A J et al., Proc Natl Acad Sci USA, 2014, 111(14): 5153-8, doi: 10.1073/pnas.1324022111; Lonberg N, Handb Exp Pharmacol, 2008, 181(181): 69-97, doi: 10.1007/978-3-540-73259-4_4; Ma B et al., “Transgenic Animals for the Generation of Human Antibodies”, in: Rüker F et al. (eds), “Introduction to Antibody Engineering”, 2021, Springer, doi: 10.1007/978-3-030-54630-4_5; WO 91/09967; WO 92/011018; WO 94/04679; WO 98/45332; or US 2021/0040182. Monoclonal antibodies can further be made by recombinant antibody library display technologies, including, e.g., by phage display, yeast display, or ribosome/mRNA display (see, e.g., Winter G et al., Annu Rev Immunol, 1994, 12:433-55, doi: 10.1146/annurev.iy. 12.040194.002245; Hammers C M et al., J Invest Dermatol, 2014, 134(2): e17, doi: 10.1038/jid.2013.521; Boder E T et al., Arch Biochem Biophys, 2012, 526(2): 99-106, doi: 10.1016/j.abb.2012.03.009; Sheehan J et al., Microbiol Spectr, 2015, 3(1): AID-0028-2014, doi: 10.1128/microbiolspec.AID-0028-2014; Feldhaus M J et al., J Immunol Methods, 2004, 290(1-2): 69-80, doi: 10.1016/j.jim.2004.04.009; Hoogenboom H R, Nat Biotechnol, 2005, 23(9): 1105-16, doi: 10.1038/nbt1126; He M et al., Expert Rev Proteomics, 2005, 2(3): 421-30, doi: 10.1586/14789450.2.3.421; U.S. Pat. Nos. 5,565,332; 5,580,717; 5,733,743; or U.S. Pat. No. 6,265,150). Each one of the documents mentioned in this paragraph is incorporated herein by reference in its entirety.

An antibody molecule typically comprises a heavy chain variable region (V_{H}) and a light chain variable region (V_{L}), which are involved in antigen binding. Such V_{H} and V_{L} regions can be further divided into (i) hypervariable regions known as “complementarity-determining regions” or “CDRs”, and (ii) more conserved regions which are also known as “framework regions” or “FRs” (or synonymously “FWs”). Typically, each V_{H} or V_{L} region is composed of three CDRs and four FRs, which are arranged in the following order (from the N-terminus to the C-terminus): FR1-CDR1-FR2-CDR2-FR3-CDR3-FR4. While it is usually evident from the context whether the CDRs or the FRs of the heavy chain or light chain variable region are referred to, CDRs and FRs can be distinguished herein with the indicator “H” or “L”. Thus, for example, the CDRs and FRs of a heavy chain variable domain can be referenced as follows (from the N-terminus to the C-terminus): [FR-H1]-(CDR-H1)-[FR-H2]-(CDR-H2)-[FR-H3]-(CDR-H3)-[FR-H4]. Likewise, the CDRs and FRs of a light chain variable domain can be referenced as follows (from the N-terminus to the C-terminus): [FR-L1]-(CDR-L1)-[FR-L2]-(CDR-L2)-[FR-L3]-(CDR-L3)-[FR-L4]. The CDRs and the framework regions can be identified or assigned using methods/numbering schemes known in the art, including, e.g., the Kabat definition, the Chothia definition (including, in particular, the so-called “pre-1989/post-1997 Chothia definition”, as described in Al-Lazikani et al., 1997 which is referenced herein below), the Martin (enhanced Chothia) definition, the AbM definition, the contact definition, or the IMGT definition; see, e.g., Kabat E A et al., “Sequences of proteins of immunological interest”, fifth edition, 1991, US Department of Health and Human Services, National Institutes of Health (NIH) publication no. 91-3242; Chothia C et al., Nature, 1989, 342(6252): 877-83, doi: 10.1038/342877a0; Chothia C et al., J Mol Biol, 1987, 196(4): 901-17, doi: 10.1016/0022-2836(87) 90412-8; Al-Lazikani B et al., J Mol Biol, 1997, 273(4): 927-48, doi: 10.1006/jmbi. 1997.1354; Almagro J C, J Mol Recognit, 2004, 17(2): 132-43, doi: 10.1002/jmr.659; Martin A C et al., Proc Natl Acad Sci USA, 1989, 86(23): 9268-72, doi: 10.1073/pnas.86.23.9268; Rees A R et al., “Antibody combining sites: structure and prediction”, in Sternberg M J E (ed.): “Protein Structure Prediction”, Oxford University Press, Oxford, 1996, 141-72; Lefranc M P, Immunol Today, 1997, 18(11): 509, doi: 10.1016/s0167-5699(97) 01163-8; Lefranc M P, Immunologist, 1999, 7(4): 132-6; Lefranc M P et al., Dev Comp Immunol, 2003, 27(1): 55-77, doi: 10.1016/s0145-305x(02)00039-3; Lefranc M P et al., Dev Comp Immunol, 2005, 29(3): 185-203, doi: 10.1016/j.dci.2004.07.003; Lefranc M P et al., Dev Comp Immunol, 2005, 29(11): 917-38, doi: 10.1016/j.dci.2005.03.003; Lefranc M P, Cold Spring Harb Protoc, 2011, 2011(6), doi: 10.1101/pdb.ip85; Lefranc M P, Cold Spring Harb Protoc, 2011, 2011(6). doi: 10.1101/pdb.ip86; or Lefranc M P et al., Nucleic Acids Res, 2015, 43 (Database issue):D413-22, doi: 10.1093/nar/gku1056; each of which is incorporated herein by reference in its entirety. Unless explicitly indicated otherwise, the CDR sequences are identified herein according to the IMGT numbering system (or “IMGT unique numbering”); see, e.g., any of the above-mentioned references by Lefranc M P.

As explained above, the anti-CCR8 antibodies according to the present invention may be, e.g., humanized antibodies or fully human antibodies. A corresponding humanized antibody typically is a human immunoglobulin (as recipient antibody), wherein the CDR sequences of said human immunoglobulin are partially or completely (preferably completely) replaced by the CDR sequences from a non-human antibody (the donor antibody having the desired binding properties), e.g., from a murine antibody. The human immunoglobulin (the recipient antibody) may be, for example, a human IgG (e.g., human IgG1, human IgG2, human IgG3, or human IgG4), a human IgM, a human IgA (e.g., human IgA1 or human IgA2), a human IgD, or a human IgE; preferably, the human immunoglobulin is a human IgG, more preferably a human IgG1 or a human IgG4, even more preferably a human IgG1. In addition, in some embodiments, one or more framework region (FR) residues of the human immunoglobulin are replaced by corresponding non-human residues. Furthermore, the humanized antibody may optionally comprise one or more amino acid residues that are found neither in the recipient antibody nor in the “imported” CDR or framework sequences but are included to further refine and optimize the antibody's performance. In some embodiments, the humanized antibody comprises at least one, preferably two variable domains from a human immunoglobulin (e.g., from human IgG, such as human IgG1 or IgG4, particularly human IgG1), wherein all CDRs correspond to those of a non-human (e.g., a murine) immunoglobulin while all (or substantially all) of the FR regions are those of the human immunoglobulin. The humanized antibody preferably also comprises a human immunoglobulin constant region (or Fc region), or at least a portion thereof. The generation of humanized antibodies may also involve affinity maturation. Methods and techniques for generating humanized antibodies are well-known in the art and include those described herein above and/or those described in: Almagro J C et al., Front Biosci, 2008, 13:1619-33, doi: 10.2741/2786; Kim J H et al., Methods Mol Biol, 2012, 907:237-45, doi: 10.1007/978-1-61779-974-7_13; Safdari Y et al., Biotechnol Genet Eng Rev, 2013, 29:175-86, doi: 10.1080/02648725.2013.801235; or Kuramochi T et al., Methods Mol Biol, 2014, 1060:123-37, doi: 10.1007/978-1-62703-586-6_7; each of which is incorporated herein by reference.

Thus, the present invention provides a humanized form (or humanized version) of any of the antibodies or antigen-binding fragments described herein, including any one of the antibodies described in the examples section as well as any one of the antibodies (or antigen-binding fragments) according to any of the options (A-1) to (A-25) described herein below. In particular, the antibody (or antigen-binding fragment) according to the invention may comprise the six CDRs as defined in any one of the options (A-1) to (A-25), and may further comprise a human acceptor framework (e.g., a human immunoglobulin framework). Exemplary humanized antibodies are also provided herein, including the antibodies having the VH and VL sequences set out in any of the options (B-19) to (B-24) below, and particularly the antibodies 1-19 to 1-26 as described in the examples section.

In some embodiments, the anti-CCR8 antibodies according to the present invention are chimeric antibodies, e.g., antibodies having a variable region (or part of variable region) from a first species (such as, e.g., mouse), and a constant region from a second species (preferably human). Typically, both the light-chain and heavy-chain variable regions of a chimeric antibody correspond to the variable regions of an antibody from one non-human mammalian species (such as, e.g., mouse, rat, or rabbit), while the constant regions of the chimeric antibody correspond to (or are homologous to) the constant regions of a human antibody. Optionally, one or more amino acid substitutions/replacements or modifications can be made in the variable region and/or the constant region. Methods and techniques for the generation of chimeric antibodies are well-known in the art and include those described herein above.

As explained above, the monoclonal antibody (or antigen-binding fragment thereof) according to the present invention specifically binds to human CCR8. The notion that an antibody “specifically binds” to a certain target antigen (or an epitope thereof) is well-known in the art. In particular, an antibody can be said to “specifically bind” to a certain target antigen (or epitope) if it binds to said target antigen (or epitope) with greater affinity, avidity, more readily, and/or with greater duration (preferably with greater affinity) than it binds to other alternative antigens. Moreover, an antibody that “specifically binds” to a certain epitope (of an antigen) may be an antibody that binds to this epitope with greater affinity, avidity, more readily, and/or with greater duration (preferably with greater affinity) than it binds to other epitopes of the same antigen. It will be understood that “specific binding” does not necessarily require exclusive binding to the corresponding target, although such exclusive (or nearly exclusive) binding is generally desirable. Accordingly, an antibody that specifically binds to a first antigen may or may not specifically bind to a second antigen (which is different from the first antigen). In some embodiments, an antibody that “specifically binds” to a target antigen does not (or does not significantly) bind to other antigens (or, analogously, an antibody that “specifically binds” to a certain epitope may not, or may not significantly, bind to other epitopes in the same antigen), which may be reflected, e.g., in that only baseline binding activity can be detected for other antigens (or other epitopes). In some embodiments, the anti-CCR8 antibodies according to the present invention may thus exhibit some (residual) binding activity for targets other than human CCR8, but only at significantly reduced levels relative to the binding activity for human CCR8. For example, the property that the monoclonal antibody (or antigen-binding fragment thereof) according to the invention “specifically binds” to human CCR8 may be characterized by the antibody (or the antigen-binding fragment) having an affinity for the target antigen (human CCR8) that is at least 10-fold, preferably at least 20-fold, more preferably at least 50-fold, even more preferably at least 100-fold, greater (i.e., more affine) than the affinity for a non-target antigen; the affinity can be determined and expressed, e.g., as a K_{D} value, whereby a lower K_{D} value indicates a greater affinity. In some embodiments, the antibody (or the antigen-binding fragment) according to the invention may exhibit no detectable binding to a non-target antigen.

As used herein, the term “epitope” refers to the site on a target antigen that is recognized and bound by an antibody. An epitope may be linear and, in that case, may typically have a length of 6 to 15 amino acid residues. Alternatively, an epitope can be conformational. The epitope to which an antibody (or an antigen-binding fragment) binds can be determined by routine methods, e.g., by epitope mapping methods, as also described further below.

The term “CCR8” refers to the CC chemokine receptor 8 (which is also known as C-C motif chemokine receptor type 8). CC chemokine receptors (CCRs) belong to the family of G protein-coupled receptors (GPCRs) which have seven transmembrane helices; they specifically bind to cytokines of the CC chemokine family. CCR8 may also be referred to as CCR-8, CY6, GPRCY6, TER1, CDw198, CKRL1, CMKBR8, CMKBRL2, or CC-CKR-8. Unless indicated otherwise or contradicted by context, “CCR8” refers to the CCR8 protein (which is encoded by the CCR8 gene). Moreover, “CCR8” refers to human CCR8 (“hCCR8”) or homologs thereof, including mammalian CCR8 homologs or non-mammalian CCR8 homologs; corresponding examples include, in particular, murine CCR8 (“mCCR8”), rat CCR8, cynomolgus monkey CCR8, rhesus macaque CCR8, chimpanzee CCR8, chicken CCR8, dog CCR8, or cattle CCR8. “CCR8” preferably refers to human CCR8. The human CCR8 gene is described, e.g., under NCBI gene ID 1237, or Ensembl ID ENSG00000179934, or HGNC gene ID 1609. The human CCR8 protein and its amino acid sequence are described, e.g., under Uniprot accession number P51685, or as NCBI reference sequence NP_005192.1. In particular, human CCR8 protein may have (or consist of) the sequence of the human isoform 1 (P51685-1) or human isoform 2 (P51685-2) as described in Uniprot. Human CCR8 may also refer to a protein encoded by the mRNA described as NCBI reference sequence NM_005201.4. Preferably, human CCR8 refers to a protein having (or consisting of) the following amino acid sequence:

(SEQ ID NO: 1)
MDYTLDLSVTTVTDYYYPDIFSSPCDAELIQTNGKLLLAVFYCLLF
VFSLLGNSLVILVLVVCKKLRSITDVYLLNLALSDLLFVFSFPFQ
TYYLLDQWVFGTVMCKVVSGFYYIGFYSSMFFITLMSVDRYLAWH
AVYALKVRTIRMGTTLCLAVWLTAIMATIPLLVFYQVASEDGVLQ
CYSFYNQQTLKWKIFTNFKMNILGLLIPFTIFMFCYIKILHQLKR
CQNHNKTKAIRLVLIVVIASLLFWVPFNVVLFLTSLHSMHILDGC
SISQQLTYATHVTEIISFTHCCVNPVIYAFVGEKFKKHLSEIFQK
SCSQIFNYLGRQMPRESCEKSSSCQQHSSRSSSVDYIL

The sequences of other mammalian or non-mammalian homologs of human CCR8 are also described in Uniprot or NCBI (or other databases known in the art). Thus, murine (mouse) CCR8 and its sequence are described, e.g., under Uniprot accession number P56484 or under NCBI reference sequence NP_031746.1; rat CCR8 and its sequence are described, e.g., under NCBI reference sequence XP_008764924.1; cynomolgus monkey CCR8 and its sequence are described, e.g., under Uniprot accession number G7NYJ2 or under NCBI reference sequence XP_015300839.1; rhesus macaque CCR8 and its sequence are described, e.g., under Uniprot accession number 097665 or under NCBI reference sequence XP_001084047.1; chimpanzee CCR8 and its sequence are described, e.g., under NCBI reference sequence XP_526178.3; chicken CCR8 and its sequence are described, e.g., under NCBI reference sequence NP_001026162.1; dog CCR8 and its sequence are described, e.g., under NCBI reference sequence XP_542719.1; cattle CCR8 and its sequence are described, e.g., under NCBI reference sequence NP_001181891.1. Different isoforms or variants of CCR8 which may exist in some species are each specifically comprised by the term CCR8. The CCR8 protein may also be subject to modifications, such as posttranslational modifications, or may be unmodified. In particular, posttranslational modifications of CCR8 have been reported, e.g., in: Gutiérrez J et al., Journal of Biological Chemistry, 2004, 279(15): 14726-33, doi: 10.1074/jbc.M309689200; or Ludeman J P et al., British Journal of Pharmacology, 2014, 171(5): 1167-79, doi: 10.1111/bph.12455). These CCR8 post-translational modifications can include tyrosine-sulfations catalyzed by enzymes such as tyrosylprotein sulfotransferase-1 or 2 (TPST-1 or TPST-2; see, e.g., Danan L M et al., J Am Soc Mass Spectrom, 2008, 19(10): 1459-66, doi: 10.1016/j.jasms.2008.06.021). Interestingly, TPST-1 and the sulfation of a chemokine receptor, e.g., CXCR4, have been associated with metastatic potential of neopharyngeal carcinoma (Xu J et al., PLOS One, 2013, 8(3): e56114, doi: 10.1371/journal.pone.0056114). Such posttranslational modifications have also been reported to have critical roles in pathological conditions as it has been previously demonstrated for another chemokine receptor, e.g., CCR5, in the context of HIV entry (Farzan M et al., Cell, 1999, 96(5): 667-76, doi: 10.1016/s0092-8674(00)80577-2). Moreover, recombinant forms or synthetic forms of CCR8 are likewise encompassed by the term CCR8. Each of the sequences described under the above-mentioned reference numbers, accession numbers or ID numbers is individually incorporated herein by reference.

As explained above, the monoclonal antibody (or antigen-binding fragment thereof) according to the present invention specifically binds to human CCR8, particularly to human CCR8 which is expressed on the surface of a cell. Preferably, the antibody (or antigen-binding fragment) specifically binds to an extracellular domain of human CCR8. Accordingly, the antibody (or antigen-binding fragment) may specifically bind to an epitope formed from any one or more extracellular portion(s) of human CCR8 (i.e., one or more of those parts of human CCR8 that extend from the plasma membrane into the extracellular space), including (i) the N-terminal extracellular portion (which extends from the N-terminus to the first transmembrane (TM) helix of the human CCR8 protein), (ii) the first extracellular loop (which connects the second TM helix to the third TM helix), (iii) the second extracellular loop (which connects the fourth TM helix to the fifth TM helix), and/or (iv) the third extracellular loop (which connects the sixth TM helix to the seventh TM helix) of human CCR8. The different portions of CCR8, including its three extracellular loops, are known in the art (see, e.g., Barington L et al., J Biol Chem, 2016, 291(31): 16208-20, doi: 10.1074/jbc.M115.706747). In some embodiments, the antibody (or antigen-binding fragment) specifically binds to the N-terminal extracellular portion of human CCR8. Accordingly, in some embodiments, the antibody (or antigen-binding fragment) specifically binds to an epitope within the N-terminal extracellular portion of human CCR8. In some embodiments, the antibody (or antigen-binding fragment) specifically binds to the first extracellular loop of human CCR8. Accordingly, in some embodiments, the antibody (or antigen-binding fragment) specifically binds to an epitope within the first extracellular loop of human CCR8. In some embodiments, the antibody (or antigen-binding fragment) specifically binds to the second extracellular loop of human CCR8. Accordingly, in some embodiments, the antibody (or antigen-binding fragment) specifically binds to an epitope within the second extracellular loop of human CCR8. In some embodiments, the antibody (or antigen-binding fragment) specifically binds to the third extracellular loop of human CCR8. Accordingly, in some embodiments, the antibody (or antigen-binding fragment) specifically binds to an epitope within the third extracellular loop of human CCR8.

Preferably, the antibody (or antigen-binding fragment) specifically binds to the N-terminal 20 to 50 amino acid residues of SEQ ID NO: 1 (i.e., the first 20 to 50 amino acid residues as counted from the N-terminus of SEQ ID NO: 1), particularly the N-terminal 30 to 40 amino acid residues of SEQ ID NO: 1, more particularly the N-terminal 34 amino acid residues of SEQ ID NO: 1. Accordingly, it is preferred that the antibody (or antigen-binding fragment) specifically binds to an epitope within the N-terminal 20 to 50 amino acid residues of SEQ ID NO: 1 (i.e., within the first 20 to 50 amino acid residues as counted from the N-terminus of SEQ ID NO: 1), particularly an epitope within the N-terminal 30 to 40 amino acid residues of SEQ ID NO: 1, more particularly an epitope within the N-terminal 34 amino acid residues of SEQ ID NO: 1.

Human CCR8 contains several tyrosine (Y) residues, particularly in its N-terminal extracellular portion, which tyrosine residues can be present in sulfated or non-sulfated form. The sulfation of tyrosine residues in a protein is a posttranslational modification where a sulfate group is added to the corresponding tyrosine residue, so that the side-chain hydroxy group (—OH) of the tyrosine residue is converted into a sulfate group (—O—SO₃H) (see, e.g., Moore K L, J Biol Chem, 2003, 278(27): 24243-6, doi: 10.1074/jbc.R300008200; or Moore K L, Proc Natl Acad Sci USA, 2009, 106(35): 14741-2, doi: 10.1073/pnas.0908376106). The sulfation of tyrosine residues can be catalyzed by enzymes such as tyrosylprotein sulfotransferase-1 or 2 (TPST-1 or TPST-2) (see, e.g., Danan L M et al., J Am Soc Mass Spectrom, 2008, 19(10): 1459-66, doi: 10.1016/j.jasms.2008.06.021). In certain types of cancer, the CCR8 expressed by tumor-infiltrating Treg cells and/or by the cancer cells may have one or more sulfated tyrosine (Y) residues, particularly in one or more of the positions corresponding to Y15, Y16 and/or Y17 of SEQ ID NO: 1, whereas these tyrosine residues may be present in non-sulfated form in other types of cancer. It is therefore desirable that the antibody (or antigen-binding fragment) according to the invention is capable of specifically binding to human CCR8, regardless of whether the corresponding tyrosine residues are present in sulfated or non-sulfated form. The capability of an antibody to specifically bind to CCR8 having sulfated or non-sulfated tyrosine residues can be determined by any suitable binding assay or experiment (e.g., as described herein below in the examples). While such binding assays can be conducted with different sulfated (or non-sulfated) forms of the complete CCR8 protein (particularly the complete human CCR8 protein), it is also possible—and typically more convenient—to use different sulfated (or non-sulfated) forms of a partial sequence of the CCR8 protein (which may also be referred to as CCR8 fragment or CCR8 peptide) that encompasses the corresponding tyrosine residue(s). For example, in such binding assays, it is possible to use a peptide consisting of a partial sequence of about 20 to about 50 amino acid residues (preferably about 30 to about 40 amino acid residues) from SEQ ID NO: 1 (preferably from the N-terminal extracellular portion of SEQ ID NO: 1), which encompasses the amino acid residues Y15, Y16 and Y17 of SEQ ID NO: 1. More preferably, a peptide comprising (or, preferably, consisting of) the N-terminal 20 to 50 amino acid residues of SEQ ID NO: 1 (i.e., the first 20 to 50 amino acid residues that SEQ ID NO: 1 starts with) can be used, even more preferably a peptide comprising (or, in particular, consisting of) the N-terminal 30 to 40 amino acid residues of SEQ ID NO: 1. Yet even more preferably, a peptide/protein comprising (or, in particular, consisting of) the 34 N-terminal amino acid residues of SEQ ID NO: 1 can be used. It will be understood that different sulfated (or non-sulfated) forms of such a peptide, wherein one or more of the tyrosine residues Y15, Y16 and Y17 is/are sulfated or non-sulfated (e.g., wherein Y17 is sulfated and wherein Y15 and Y16 are each independently sulfated or non-sulfated), are typically employed in binding experiments in order to determine the capability of an antibody to specifically bind to the different forms of the peptide and, consequently, to determine the capability of the antibody to specifically bind to sulfated and/or non-sulfated forms of CCR8. The preparation of corresponding exemplary sulfated and non-sulfated CCR8 peptides is described in Example 1. Accordingly, in some embodiments, one or more (e.g., all) of the peptides as described in Example 1 can be used. Other sulfated and non-sulfated CCR8 peptides can be prepared, e.g., in accordance with, or in analogy to, the procedures described in: Seibert C et al., Methods Enzymol, 2016, 570:357-88, doi: 10.1016/bs.mie.2015.09.004; which is incorporated herein by reference.

Thus, in some embodiments, the antibody (or antigen-binding fragment) according to the invention specifically binds to one or more (preferably two or more; more preferably three or more; even more preferably all) of the following:
human CCR8 or a partial sequence thereof, wherein said partial sequence comprises or consists of the amino acid sequence MDYTLDLSVTTVTDYYYPDIFSSPCDAELIQTNG (SEQ ID NO: 2), having a sulfated tyrosine residue in position Y17 and non-sulfated tyrosine residues in the positions Y15 and Y16;
human CCR8 or a partial sequence thereof, wherein said partial sequence comprises or consists of the amino acid sequence MDYTLDLSVTTVTDYYYPDIFSSPCDAELIQTNG (SEQ ID NO: 3), having sulfated tyrosine residues in the positions Y15 and Y17 and a non-sulfated tyrosine residue in position Y16;
human CCR8 or a partial sequence thereof, wherein said partial sequence comprises or consists of the amino acid sequence MDYTLDLSVTTVTDYYYPDIFSSPCDAELIQTNG (SEQ ID NO: 4), having sulfated tyrosine residues in the positions Y16 and Y17 and a non-sulfated tyrosine residue in position Y15; and
human CCR8 or a partial sequence thereof, wherein said partial sequence comprises or consists of the amino acid sequence MDYTLDLSVTTVTDYYYPDIFSSPCDAELIQTNG (SEQ ID NO: 5), having sulfated tyrosine residues in the positions Y15, Y16 and Y17.

Accordingly, the present invention provides a monoclonal antibody or an antigen-binding fragment thereof, which specifically binds to human CCR8 and is an antagonist, particularly an insurmountable antagonist, of the CCL1-CCR8 signaling pathway, wherein said antibody or said antigen-binding fragment specifically binds to one or more (preferably two or more; more preferably three or more; even more preferably all) of the following:
human CCR8 or a partial sequence thereof, wherein said partial sequence comprises or consists of SEQ ID NO: 2, having a sulfated tyrosine residue in position Y17 and non-sulfated tyrosine residues in the positions Y15 and Y16;
human CCR8 or a partial sequence thereof, wherein said partial sequence comprises or consists of SEQ ID NO: 3, having sulfated tyrosine residues in the positions Y15 and Y17 and a non-sulfated tyrosine residue in position Y16;
human CCR8 or a partial sequence thereof, wherein said partial sequence comprises or consists of SEQ ID NO: 4, having sulfated tyrosine residues in the positions Y16 and Y17 and a non-sulfated tyrosine residue in position Y15; and
human CCR8 or a partial sequence thereof, wherein said partial sequence comprises or consists of SEQ ID NO: 5, having sulfated tyrosine residues in the positions Y15, Y16 and Y17.

It is preferred that the antibody (or antigen-binding fragment) according to the invention specifically binds to one or more (preferably two or more; more preferably three or more; even more preferably all) of the following:
a peptide consisting of the sequence MDYTLDLSVTTVTDYYYPDIFSSPCDAELIQTNG (SEQ ID NO: 2), having a sulfated tyrosine residue in position Y17 and non-sulfated tyrosine residues in the positions Y15 and Y16;
a peptide consisting of the sequence MDYTLDLSVTTVTDYYYPDIFSSPCDAELIQTNG (SEQ ID NO: 3), having sulfated tyrosine residues in the positions Y15 and Y17 and a non-sulfated tyrosine residue in position Y16;
a peptide consisting of the sequence MDYTLDLSVTTVTDYYYPDIFSSPCDAELIQTNG (SEQ ID NO: 4), having sulfated tyrosine residues in the positions Y16 and Y17 and a non-sulfated tyrosine residue in position Y15; and
a peptide consisting of the sequence MDYTLDLSVTTVTDYYYPDIFSSPCDAELIQTNG (SEQ ID NO: 5), having sulfated tyrosine residues in the positions Y15, Y16 and Y17.

More preferably, the antibody (or antigen-binding fragment) according to the invention specifically binds to one or more non-sulfated forms of human CCR8 (i.e., one or more forms/variants of human CCR8 wherein at least one of the tyrosine residues in the positions Y15, Y16 and Y17 is non-sulfated, particularly wherein at least one of the tyrosine residues in the positions Y15 and Y16 is non-sulfated). It is thus particularly preferred that the antibody (or antigen-binding fragment) according to the invention specifically binds to one or more (preferably two or more; more preferably all) of the following:
human CCR8 or a partial sequence thereof, wherein said partial sequence comprises or consists of the amino acid sequence MDYTLDLSVTTVTDYYYPDIFSSPCDAELIQTNG (SEQ ID NO: 2), having a sulfated tyrosine residue in position Y17 and non-sulfated tyrosine residues in the positions Y15 and Y16;
human CCR8 or a partial sequence thereof, wherein said partial sequence comprises or consists of the amino acid sequence MDYTLDLSVTTVTDYYYPDIFSSPCDAELIQTNG (SEQ ID NO: 3), having sulfated tyrosine residues in the positions Y15 and Y17 and a non-sulfated tyrosine residue in position Y16; and
human CCR8 or a partial sequence thereof, wherein said partial sequence comprises or consists of the amino acid sequence MDYTLDLSVTTVTDYYYPDIFSSPCDAELIQTNG (SEQ ID NO: 4), having sulfated tyrosine residues in the positions Y16 and Y17 and a non-sulfated tyrosine residue in position Y15.

Accordingly, in particularly preferred embodiments, the present invention provides a monoclonal antibody or an antigen-binding fragment thereof, which specifically binds to human CCR8 and is an antagonist, particularly an insurmountable antagonist, of the CCL1-CCR8 signaling pathway, wherein said antibody or said antigen-binding fragment specifically binds to one or more (preferably two or more; more preferably all) of the following:
human CCR8 or a partial sequence thereof, wherein said partial sequence comprises or consists of SEQ ID NO: 2, having a sulfated tyrosine residue in position Y17 and non-sulfated tyrosine residues in the positions Y15 and Y16;
human CCR8 or a partial sequence thereof, wherein said partial sequence comprises or consists of SEQ ID NO: 3, having sulfated tyrosine residues in the positions Y15 and Y17 and a non-sulfated tyrosine residue in position Y16; and
human CCR8 or a partial sequence thereof, wherein said partial sequence comprises or consists of SEQ ID NO: 4, having sulfated tyrosine residues in the positions Y16 and Y17 and a non-sulfated tyrosine residue in position Y15.

It is particularly preferred that the antibody (or antigen-binding fragment) according to the invention specifically binds to one or more (preferably two or more; more preferably all) of the following:
a peptide consisting of the sequence MDYTLDLSVTTVTDYYYPDIFSSPCDAELIQTNG (SEQ ID NO: 2), having a sulfated tyrosine residue in position Y17 and non-sulfated tyrosine residues in the positions Y15 and Y16;
a peptide consisting of the sequence MDYTLDLSVTTVTDYYYPDIFSSPCDAELIQTNG (SEQ ID NO: 3), having sulfated tyrosine residues in the positions Y15 and Y17 and a non-sulfated tyrosine residue in position Y16; and
a peptide consisting of the sequence MDYTLDLSVTTVTDYYYPDIFSSPCDAELIQTNG (SEQ ID NO: 4), having sulfated tyrosine residues in the positions Y16 and Y17 and a non-sulfated tyrosine residue in position Y15.

Preferably, the antibody or antigen-binding fragment according to the invention binds to human CCR8 with a dissociation constant (K_{D}) of about 100 nM or less, more preferably with a K_{D} of about 50 nM or less, even more preferably with a K_{D} of about 30 nM or less, even more preferably with a K_{D} of about 20 nM or less, even more preferably with a K_{D} of about 15 nM or less, yet even more preferably with a K_{D} of about 10 nM or less. In particular, it is preferred that the antibody or antigen-binding fragment according to the invention specifically binds to its target antigen (e.g., any specific antigen mentioned herein above or below) with a K_{D} of about 100 nM or less, more preferably about 50 nM or less, even more preferably about 30 nM or less, even more preferably about 20 nM or less, even more preferably about 15 nM or less, yet even more preferably about 10 nM or less. The dissociation constant (K_{D}) is commonly used as a measure for the affinity (or the binding activity) of an antibody, particularly for an antibody's affinity for its target antigen. As understood in the art, the K_{D} value of an antibody for a target is inversely proportional to its affinity (or binding activity) for that target. Therefore, an antibody or antibody binding fragment that specifically binds to its antigen, e.g., with a K_{D} of “at least” 50 nM or with a K_{D} of 50 nM “or better” will generally be understood as binding to its antigen with a K_{D} of 50 nM “or less”. Alternatively, or additionally, the antibody or antigen-binding fragment according to the invention may specifically binds to its target antigen with an association rate (kₐ or kₒₙ) of about 1×10⁵ M⁻¹ s⁻¹ or greater, preferably with a kₐ of about 2×10⁵ M⁻¹ s⁻¹ or greater, more preferably with a kₐ of about 5×10⁵ M⁻¹ s⁻¹ or greater.

The binding properties, including the binding specificity or affinity, of the antibodies and antigen-binding fragments provided herein may be established by any suitable method known in the art and/or any method as described herein, which allows the quantification of binding parameters. Methods for analyzing the binding specificity and binding parameters of an antibody or antigen-binding fragment are described, e.g. in: Harlow E et al., “Using Antibodies: A Laboratory Manual”, 1999, Cold Spring Harbor Laboratory Press; or Greenfield E A, “Antibodies: A Laboratory Manual”, second edition, 2014, Cold Spring Harbor Laboratory Press (which are each incorporated herein by reference). Non-limiting examples of suitable studies include binding studies and/or blocking/competition studies with structurally and/or functionally closely related molecules. These studies can be carried out by methods such as, e.g., FACS analysis, flow cytometric titration analysis (FACS titration), surface plasmon resonance (SPR; e.g., using BIAcore™), isothermal titration calorimetry (ITC), fluorescence titration, or by radiolabeled ligand binding assays. Further methods include, e.g., any of Western blots, ELISA (e.g., competition ELISA), RIA, ECL, and IRMA tests. The specificity and selectivity of the antibodies and antigen-binding fragments of the invention are preferably determined by measuring antibody affinity, e.g., by determining the dissociation constant (K_{D}). Where the K_{D} is determined, it is preferably measured using surface plasmon resonance spectroscopy, e.g., with BIAcore®. In particular, the dissociation constant (K_{D}) may be determined using a BIAcore® surface plasmon resonance assay, whereby the antigen (e.g., human CCR8) is immobilized on biosensor chips using an injection flow rate of about 5 μl/min and a temperature of about 25° C. to obtain a density of about 10 response units (RU), and whereby the antibody (or antigen-binding fragment) is subsequently injected at a flow rate of about 25 μl/min and a temperature of about 25° C. In addition, or alternatively, the dissociation constant (K_{D}) may be determined using a BIAcore® surface plasmon resonance assay, following the approach described in Murphy M et al., Curr Protoc Protein Sci, 2006, Chapter 19: Unit 19.14, doi: 10.1002/0471142301.ps1914s45.

In preferred embodiments, the antibody (or antigen-binding fragment) according to the invention specifically binds to human CCR8 and to at least one other mammalian (non-human) CCR8, e.g., it specifically binds to human CCR8 and to cynomolgus CCR8, or it specifically binds to human CCR8 and to murine CCR8. Such species cross-reactivity is advantageous, as it considerably facilitates the development of the corresponding antibody (or antigen-binding fragment) into a medicinal product.

The term “antagonist”, as used herein when referring to an antagonist of a specific receptor or an antagonist of a specific receptor signaling pathway, denotes a substance/agent that binds to the corresponding receptor and inhibits, blocks, prevents or reduces the corresponding biological response, i.e., a substance/agent which inhibits, blocks, prevents or reduces the signal transduction that would otherwise be elicited by the binding of a ligand to the receptor. For example, an “antagonist of the CCL1-CCR8 signaling pathway” refers to a substance/agent (e.g., an antibody or an or antigen-binding fragment thereof) that inhibits, blocks, prevents or reduces (or, in other words, is capable of inhibiting, blocking, preventing or reducing) the signal transduction elicited by the binding of the ligand CCL1 to the receptor CCR8 (preferably the binding of human CCL1 to human CCR8).

The term “insurmountable antagonist”, as used herein, refers to an antagonist, as defined above, whose effect on the corresponding receptor depresses/reduces the maximal response of an agonist (see, e.g., Kenakin T, “A Pharmacology Primer”, Fifth Edition, Academic Press (2018)). Accordingly, even the addition of the agonist/ligand in excess (relative to the antagonist) does not fully overcome the inhibiting/blocking effect of the antagonist on the receptor. In other words, the insurmountable antagonist reduces the magnitude of the maximal response that can be elicited by the corresponding agonist/ligand (which can be determined, e.g., by establishing dose-response curves with different doses/concentrations of agonist/ligand). In principle, an insurmountable antagonist may bind to an allosteric site of the receptor (i.e., to a binding site which is different from the active site of the receptor where the ligand binds; in this case, the antagonist does not compete with the ligand for binding to the active site of the receptor), or alternatively it may bind to the active site of the receptor; the present invention specifically and individually relates to each of these meanings.

The antagonist activity (or antagonistic effect) of the antibody or antigen-binding fragment according to the present invention on the CCL1-CCR8 signaling pathway can be determined using methods or assays known in the art or approaches based on such known methods or assays (see, e.g., Liu L et al., Biochem Pharmacol, 2021, 188:114565, doi: 10.1016/j.bcp.2021.114565; or Avet C et al., Elife, 2022, 11: e74101, doi: 10.7554/eLife.74101; each of which is incorporated herein by reference). For example, the property of the antibody or antigen-binding fragment according to the invention of being an antagonist of the CCL1-CCR8 signaling pathway can be determined by testing the capability of the antibody or antigen-binding fragment to inhibit, block, prevent or reduce the signal transduction elicited by the binding of the ligand CCL1 (particularly human CCL1) to the receptor CCR8 (particularly human CCR8), preferably by testing the capability to inhibit, block, prevent or reduce the CCR8-Gi₂ signaling induced by CCL1. By way of example, any one of the assays described in Example 14 may be used in order to determine whether an antibody or antigen-binding fragment is an antagonist of the CCL1-CCR8 signaling pathway. When using an assay as described in Example 14 (or any other assay or method, e.g., as mentioned herein above), an antibody or antigen-binding fragment can be confirmed to be an antagonist of the CCL1-CCR8 signaling pathway if any level of inhibition, reduction, prevention or blocking of CCL1-CCR8 signaling is observed. Preferably, however, the antibody or antigen-binding fragment according to the invention provides at least a 10% inhibition of the CCL1-CCR8 signaling pathway (i.e., reduces CCL1-CCR8 signaling by at least 10%, relative to the level of CCL1-CCR8 signaling without said antibody or antigen-binding fragment (“positive control”)), more preferably at least a 20% inhibition, even more preferably at least a 30% inhibition, even more preferably at least a 40% inhibition, even more preferably at least a 50% inhibition, even more preferably at least a 60% inhibition, even more preferably at least a 70% inhibition, even more preferably at least an 80% inhibition, yet even more preferably at least a 90% inhibition of the CCL1-CCR8 signaling pathway; such percent inhibition can be determined using any of the aforementioned methods/assays, such as, e.g., an assay as described in Example 14. In the case of an “insurmountable” antagonist of the CCL1-CCR8 signaling pathway, the inhibition (or reduction, prevention or blocking) of the CCL1-CCR8 signaling pathway cannot be reversed completely by addition of the ligand CCL1 in excess (e.g., in about 10-fold molar excess, preferably in about 50-fold molar excess, more preferably in about 100-fold molar excess, even more preferably in about 180-fold molar excess, yet even more preferably in about 940-fold molar excess) relative to the antibody or antigen-binding fragment. The property/characteristic of an antibody or antigen-binding fragment of being an insurmountable antagonist of the CCL1-CCR8 signaling pathway can be determined, e.g., using any one of the assays described in Example 14 (e.g., the assay according to protocol #1, #2, #3 or #4 as described in Example 14).

As explained above, the antibody or antigen-binding fragment according to the invention is an antagonist (preferably an insurmountable antagonist) of the CCL1-CCR8 signaling pathway. In particular, it is preferred that the antibody or antigen-binding fragment is an antagonist (more preferably an insurmountable antagonist) of CCL1-induced CCR8-Gi₂ signaling. Thus, it is particularly preferred that the antibody or antigen-binding fragment according to the invention is an insurmountable antagonist of CCL1-induced CCR8-Gi₂ signaling.

The antagonistic effect of the antibody or antigen-binding fragment according to the invention on CCL1-induced CCR8-Gi₂ signaling can be determined using methods or assays known in the art or approaches based on such known methods or assays (including, e.g., any of the assays/methods described herein above). For example, the property of the antibody or antigen-binding fragment according to the invention of being an antagonist of CCL1-induced CCR8-Gi₂ signaling can be determined by testing the capability of the antibody or antigen-binding fragment to inhibit, block, prevent or reduce the CCR8-Gi₂ signaling induced by the binding of the ligand CCL1 (particularly human CCL1). By way of example, any one of the assays described in Example 14 may be used in order to determine whether an antibody or antigen-binding fragment is an antagonist of CCL1-induced CCR8-Gi₂ signaling. When using an assay as described in Example 14 (or any other assay or method, e.g., as mentioned herein above), an antibody or antigen-binding fragment can be confirmed to be an antagonist of CCL1-induced CCR8-Gi₂ signaling if any level of inhibition, reduction, prevention or blocking of CCL1-induced CCR8-Gi₂ signaling is observed. Preferably, however, the antibody or antigen-binding fragment according to the invention provides at least a 10% inhibition of CCL1-induced CCR8-Gi₂ signaling (i.e., reduces the signaling by at least 10%, relative to the level of signaling without said antibody or antigen-binding fragment (“positive control”)), more preferably at least a 20% inhibition, even more preferably at least a 30% inhibition, even more preferably at least a 40% inhibition, even more preferably at least a 50% inhibition, even more preferably at least a 60% inhibition, even more preferably at least a 70% inhibition, even more preferably at least an 80% inhibition, yet even more preferably at least a 90% inhibition of CCL1-induced CCR8-Gi₂ signaling; such percent inhibition can be determined using any of the aforementioned methods/assays, such as, e.g., an assay as described in Example 14. In the case of an “insurmountable” antagonist of CCL1-induced CCR8-Gi₂ signaling, the inhibition (or reduction, prevention or blocking) of the corresponding signaling pathway (i.e., CCL1-induced CCR8-Gi₂ signaling) cannot be reversed completely by addition of the ligand CCL1 in excess (e.g., in about 10-fold molar excess, preferably in about 50-fold molar excess, more preferably in about 100-fold molar excess, even more preferably in about 180-fold molar excess, yet even more preferably in about 940-fold molar excess) relative to the antibody or antigen-binding fragment. In particular, the property/characteristic of an antibody or antigen-binding fragment of being an insurmountable antagonist of CCL1-induced CCR8-Gi₂ signaling can be determined, e.g., using any one of the assays described in Example 14 (e.g., the assay according to protocol #1, #2, #3 or #4 as described in Example 14).

It is preferred that the antibody or antigen-binding fragment according to the invention inhibits the binding of CCL1 to CCR8 (preferably the binding of human CCL1 to human CCR8) with an IC₅₀ of about 20 nM or less (e.g., about 1 nM to about 20 nM), more preferably with an IC₅₀ of about 13 nM or less, even more preferably with an IC₅₀ of about 10 nM or less, yet even more preferably with an IC₅₀ of about 6 nM or less. In particular, it is preferred that the antibody (or antigen-binding fragment) according to the invention inhibits the binding of hCCL1 to hCCR8 expressed at a cell surface (e.g., expressed at the cell surface on hCCR8-transfected CHO cells) with an IC₅₀ of about 20 nM or less (e.g., about 1 nM to about 20 nM), more preferably with an IC₅₀ of about 13 nM or less, even more preferably with an IC₅₀ of about 10 nM or less, yet even more preferably with an IC₅₀ of about 6 nM or less. This inhibition can be determined, e.g., using the assay described in Example 13.

In preferred embodiments, the antibody (or antigen-binding fragment) according to the invention comprises one or more of the CDRs (preferably all three heavy-chain CDRs and/or all three light-chain CDRs; more preferably all six CDRs) of any one of the exemplary antibodies described in the examples section herein below. In particular, it is preferred that the antibody (or antigen-binding fragment) according to the invention comprises:
(A-1) a heavy chain variable domain (VH) comprising a CDR-H1 having the amino acid sequence PYAMN (SEQ ID NO: 6), a CDR-H2 having the amino acid sequence RIRSKSNNYATYYADSVKD (SEQ ID NO: 7), and a CDR-H3 having the amino acid sequence GREAYYRYDGDYYAMDY (SEQ ID NO: 8); and/or (preferably; and)
   a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(A-2) a heavy chain variable domain (VH) comprising a CDR-H1 having the amino acid sequence TYAMN (SEQ ID NO: 12), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and/or (preferably; and)
   a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(A-3) a heavy chain variable domain (VH) comprising a CDR-H1 having the amino acid sequence EYTIH (SEQ ID NO: 14), a CDR-H2 having the amino acid sequence GINPNGDTRYDQKFKG (SEQ ID NO: 15), and a CDR-H3 having the amino acid sequence VARFYGISPYAMDY (SEQ ID NO: 16); and/or (preferably; and)
   a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQSTHVPYT (SEQ ID NO: 19); or
(A-4) a heavy chain variable domain (VH) comprising a CDR-H1 having the amino acid sequence AYAMN (SEQ ID NO: 20), a CDR-H2 having the amino acid sequence RIRSKSNDYATYYGDSVKD (SEQ ID NO: 21), and a CDR-H3 having the amino acid sequence GREAYYRYDGGYYAMDY (SEQ ID NO: 13); and/or (preferably; and)
   a light chain variable domain (VL) comprising a CDR-L1 having the amino acid sequence TSSKSLLHSNGNTYLY (SEQ ID NO: 22), a CDR-L2 having the amino acid sequence RMSNLAS (SEQ ID NO: 10), and a CDR-L3 having the amino acid sequence MQHREYPFT (SEQ ID NO: 23); or
(A-5) a heavy chain variable domain (VH) comprising a CDR-H1 having the amino acid sequence DYVVT (SEQ ID NO: 24), a CDR-H2 having the amino acid sequence VIWGGGNTYYNSDLKS (SEQ ID NO: 25), and a CDR-H3 having the amino acid sequence RHRDYALDY (SEQ ID NO: 26); and/or (preferably; and)
   a light chain variable domain (VL) comprising a CDR-L1 having the amino acid sequence RSSQSLVYSNGNTYLH (SEQ ID NO: 27), a CDR-L2 having the amino acid sequence KVSNRFS (SEQ ID NO: 18), and a CDR-L3 having the amino acid sequence SQSTYVPPT (SEQ ID NO: 28); or
(A-6) a heavy chain variable domain (VH) comprising a CDR-H1 having the amino acid sequence DYTLH (SEQ ID NO: 29), a CDR-H2 having the amino acid sequence GITPKNGDTRYDPRFKD (SEQ ID NO: 30), and a CDR-H3 having the amino acid sequence VARFYGVSPYAMDY (SEQ ID NO: 31); and/or (preferably; and)
   a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQTTHVPYT (SEQ ID NO: 32); or
(A-7) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PDGFSPFVY (SEQ ID NO: 35); and/or (preferably; and)
   a light chain variable domain (VL) comprising a CDR-L1 having the amino acid sequence KASQDINSYLS (SEQ ID NO: 36), a CDR-L2 having the amino acid sequence RANRLVD (SEQ ID NO: 37), and a CDR-L3 having the amino acid sequence LQYGEFPPT (SEQ ID NO: 38); or
(A-8) a heavy chain variable domain (VH) comprising a CDR-H1 having the amino acid sequence AYAMN (SEQ ID NO: 20), a CDR-H2 having the amino acid sequence RIRSKSNNYATYYGDSVKD (SEQ ID NO: 39), and a CDR-H3 having the amino acid sequence GREAYYRYDGDYYAMDY (SEQ ID NO: 8); and/or (preferably; and)
   a light chain variable domain (VL) comprising a CDR-L1 having the amino acid sequence KSSQSLLNSSNQKNYLA (SEQ ID NO: 40), a CDR-L2 having the amino acid sequence FASTRES (SEQ ID NO: 41), and a CDR-L3 having the amino acid sequence QQHYSTPYT (SEQ ID NO: 42); or
(A-9) a heavy chain variable domain (VH) comprising a CDR-H1 having the amino acid sequence AYAMN (SEQ ID NO: 20), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and/or (preferably; and)
   a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(A-10) a heavy chain variable domain (VH) comprising a CDR-H1 having the amino acid sequence EYTIH (SEQ ID NO: 14), a CDR-H2 having the amino acid sequence GINPNNGNTRYDQKFKG (SEQ ID NO: 43), and a CDR-H3 having the amino acid sequence VARSSGSGPYAMDY (SEQ ID NO: 44); and/or (preferably; and)
   a light chain variable domain (VL) comprising a CDR-L1 having the amino acid sequence RCTQSLLHTNGDTYLH (SEQ ID NO: 45), a CDR-L2 having the amino acid sequence KVSNRFS (SEQ ID NO: 18), and a CDR-L3 having the amino acid sequence SQSTHVPYT (SEQ ID NO: 19); or
(A-11) a heavy chain variable domain (VH) comprising a CDR-H1 having the amino acid sequence GYNMH (SEQ ID NO: 46), a CDR-H2 having the amino acid sequence AISPGKGDTSYNLKFKG (SEQ ID NO: 47), and a CDR-H3 having the amino acid sequence SGGTPFAY (SEQ ID NO: 48); and/or (preferably; and)
   a light chain variable domain (VL) comprising a CDR-L1 having the amino acid sequence RSSQSLVHSNGDTYLY (SEQ ID NO: 49), a CDR-L2 having the amino acid sequence KVSNRFS (SEQ ID NO: 18), and a CDR-L3 having the amino acid sequence SQSTHVPYT (SEQ ID NO: 19); or
(A-12) a heavy chain variable domain (VH) comprising a CDR-H1 having the amino acid sequence ANAMN (SEQ ID NO: 50), a CDR-H2 having the amino acid sequence RIRSKSNNYATYYADSVKD (SEQ ID NO: 7), and a CDR-H3 having the amino acid sequence GSDNYIFYAMDY (SEQ ID NO: 51); and/or (preferably; and)
   a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(A-13) a heavy chain variable domain (VH) comprising a CDR-H1 having the amino acid sequence TNAMN (SEQ ID NO: 52), a CDR-H2 having the amino acid sequence RIRSKSNYYATYYADSVKD (SEQ ID NO: 53), and a CDR-H3 having the amino acid sequence GREMGNYYSMDY (SEQ ID NO: 54); and/or (preferably; and)
   a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(A-14) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PTYPGSSGFAY (SEQ ID NO: 55); and/or (preferably; and)
   a light chain variable domain (VL) comprising a CDR-L1 having the amino acid sequence RASQDISNYLN (SEQ ID NO: 56), a CDR-L2 having the amino acid sequence YTSRLHS (SEQ ID NO: 57), and a CDR-L3 having the amino acid sequence QQGNKFPPT (SEQ ID NO: 58); or
(A-15) a heavy chain variable domain (VH) comprising a CDR-H1 having the amino acid sequence EYTMH (SEQ ID NO: 59), a CDR-H2 having the amino acid sequence GINPNNGDTNYNQKFMG (SEQ ID NO: 60), and a CDR-H3 having the amino acid sequence RLLRRGAMDY (SEQ ID NO: 61); and/or (preferably; and)
   a light chain variable domain (VL) comprising a CDR-L1 having the amino acid sequence RASENVEYYGTSLMQ (SEQ ID NO: 62), a CDR-L2 having the amino acid sequence AASNVDS (SEQ ID NO: 63), and a CDR-L3 having the amino acid sequence QQSRKVPWT (SEQ ID NO: 64); or
(A-16) a heavy chain variable domain (VH) comprising a CDR-H1 having the amino acid sequence GSYMH (SEQ ID NO: 65), a CDR-H2 having the amino acid sequence RINPYNGATSYNQNFKD (SEQ ID NO: 66), and a CDR-H3 having the amino acid sequence TLLRLLDY (SEQ ID NO: 67); and/or (preferably; and) a light chain variable domain (VL) comprising a CDR-L1 having the amino acid sequence RSSKSLLHSNGITYLY (SEQ ID NO: 68), a CDR-L2 having the amino acid sequence QMSNLAS (SEQ ID NO: 69), and a CDR-L3 having the amino acid sequence AQNLELPWT (SEQ ID NO: 70); or
(A-17) a heavy chain variable domain (VH) comprising a CDR-H1 having the amino acid sequence SYWMH (SEQ ID NO: 71), a CDR-H2 having the amino acid sequence NIWPGSASTNYDEKFKN (SEQ ID NO: 72), and a CDR-H3 having the amino acid sequence GGKGAMDY (SEQ ID NO: 73); and/or (preferably; and)
   a light chain variable domain (VL) comprising a CDR-L1 having the amino acid sequence RSSQSLENSYGNTYLN (SEQ ID NO: 74), a CDR-L2 having the amino acid sequence RVSNRFS (SEQ ID NO: 75), and a CDR-L3 having the amino acid sequence LQVTHVPPT (SEQ ID NO: 76); or
(A-18) a heavy chain variable domain (VH) comprising a CDR-H1 having the amino acid sequence TYAMH (SEQ ID NO: 77), a CDR-H2 having the amino acid sequence HINPSSGYSNYNQKFKD (SEQ ID NO: 78), and a CDR-H3 having the amino acid sequence SEVRRGYFDV (SEQ ID NO: 79); and/or (preferably; and)
   a light chain variable domain (VL) comprising a CDR-L1 having the amino acid sequence RTSQDIRNYLN (SEQ ID NO: 80), a CDR-L2 having the amino acid sequence YTSRLHS (SEQ ID NO: 57), and a CDR-L3 having the amino acid sequence QQGNTLPPT (SEQ ID NO: 81); or
(A-19) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDV (SEQ ID NO: 124); and/or (preferably; and)
   a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11); or
(A-20) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and/or (preferably; and)
   a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23); or
(A-21) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and/or (preferably; and)
   a light chain variable domain (VL) comprising a CDR-L1 having the amino acid sequence KSLLHSNGNTY (SEQ ID NO: 125), a CDR-L2 having the amino acid sequence RVS, and a CDR-L3 having the amino acid sequence MQHREYPFT (SEQ ID NO: 23); or
(A-22) a heavy chain variable domain (VH) comprising a CDR-H1 having the amino acid sequence GYTFTEYT (SEQ ID NO: 128), a CDR-H2 having the amino acid sequence INPNNGNT (SEQ ID NO: 129), and a CDR-H3 having the amino acid sequence ARVARSSGSGPYAMDY (SEQ ID NO: 130); and/or (preferably; and)
   a light chain variable domain (VL) comprising a CDR-L1 having the amino acid sequence QSLLHTNGDTY (SEQ ID NO: 131), a CDR-L2 having the amino acid sequence KVS, and a CDR-L3 having the amino acid sequence SQSTHVPYT (SEQ ID NO: 19); or
(A-23) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDY (SEQ ID NO: 133); and/or (preferably; and)
   a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23); or
(A-24) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDY (SEQ ID NO: 133); and/or (preferably; and)
   a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11); or
(A-25) a heavy chain variable domain (VH) comprising a CDR-H1 having the amino acid sequence GFTFSNYR (SEQ ID NO: 134), a CDR-H2 having the amino acid sequence IKVKSDNYGA (SEQ ID NO: 135), and a CDR-H3 having the amino acid sequence SSPTYPGSSGFAY (SEQ ID NO: 136); and/or (preferably; and)
   a light chain variable domain (VL) comprising a CDR-L1 having the amino acid sequence QDISNY (SEQ ID NO: 137), a CDR-L2 having the amino acid sequence YTS, and a CDR-L3 having the amino acid sequence QQGNKFPPT (SEQ ID NO: 58).

The above-mentioned CDR sequences (according to IMGT numbering) are also summarized in the following table:

| | | | SEQ | |
|---|---|---|---|---|
| | | | ID | |
| | Antibody | CDR | NO: | Amino acid sequence |
| (A-1) | 1-1 | CDR-H1 | 6 | PYAMN |
| | | CDR-H2 | 7 | RIRSKSNNYATYYADSVKD |
| | | CDR-H3 | 8 | GREAYYRYDGDYYAMDY |
| | | CDR-L1 | 9 | RSSKSLLHSNGNTYLY |
| | | CDR-L2 | 10 | RMSNLAS |
| | | CDR-L3 | 11 | MQHLEYPFT |
| (A-2) | 1-2 | CDR-H1 | 12 | TYAMN |
| | | CDR-H2 | 7 | RIRSKSNNYATYYADSVKD |
| | | CDR-H3 | 13 | GREAYYRYDGGYYAMDY |
| | | CDR-L1 | 9 | RSSKSLLHSNGNTYLY |
| | | CDR-L2 | 10 | RMSNLAS |
| | | CDR-L3 | 11 | MQHLEYPFT |
| (A-3) | 1-3 | CDR-H1 | 14 | EYTIH |
| | | CDR-H2 | 15 | GINPNGDTRYDQKFKG |
| | | CDR-H3 | 16 | VARFYGISPYAMDY |
| | | CDR-L1 | 17 | RCTQSLLHSNGDTYLH |
| | | CDR-L2 | 18 | KVSNRFS |
| | | CDR-L3 | 19 | SQSTHVPYT |
| (A-4) | 1-4 | CDR-H1 | 20 | AYAMN |
| | | CDR-H2 | 21 | RIRSKSNDYATYYGDSVKD |
| | | CDR-H3 | 13 | GREAYYRYDGGYYAMDY |
| | | CDR-L1 | 22 | TSSKSLLHSNGNTYLY |
| | | CDR-L2 | 10 | RMSNLAS |
| | | CDR-L3 | 23 | MQHREYPFT |
| (A-5) | 1-5 | CDR-H1 | 24 | DYVVT |
| | | CDR-H2 | 25 | VIWGGGNTYYNSDLKS |
| | | CDR-H3 | 26 | RHRDYALDY |
| | | CDR-L1 | 27 | RSSQSLVYSNGNTYLH |
| | | CDR-L2 | 18 | KVSNRFS |
| | | CDR-L3 | 28 | SQSTYVPPT |
| (A-6) | 1-6 | CDR-H1 | 29 | DYTLH |
| | | CDR-H2 | 30 | GITPKNGDTRYDPRFKD |
| | | CDR-H3 | 31 | VARFYGVSPYAMDY |
| | | CDR-L1 | 17 | RCTQSLLHSNGDTYLH |
| | | CDR-L2 | 18 | KVSNRFS |
| | | CDR-L3 | 32 | SQTTHVPYT |
| (A-7) | 1-7 | CDR-H1 | 33 | NYRMH |
| | | CDR-H2 | 34 | VIKVKSDNYGANYAESVKG |
| | | CDR-H3 | 35 | PDGFSPFVY |
| | | CDR-L1 | 36 | KASQDINSYLS |
| | | CDR-L2 | 37 | RANRLVD |
| | | CDR-L3 | 38 | LQYGEFPPT |
| (A-8) | 1-8 | CDR-H1 | 20 | AYAMN |
| | | CDR-H2 | 39 | RIRSKSNNYATYYGDSVKD |
| | | CDR-H3 | 8 | GREAYYRYDGDYYAMDY |
| | | CDR-L1 | 40 | KSSQSLLNSSNQKNYLA |
| | | CDR-L2 | 41 | FASTRES |
| | | CDR-L3 | 42 | QQHYSTPYT |
| (A-9) | 1-9 | CDR-H1 | 20 | AYAMN |
| | | CDR-H2 | 7 | RIRSKSNNYATYYADSVKD |
| | | CDR-H3 | 13 | GREAYYRYDGGYYAMDY |
| | | CDR-L1 | 9 | RSSKSLLHSNGNTYLY |
| | | CDR-L2 | 10 | RMSNLAS |
| | | CDR-L3 | 11 | MQHLEYPFT |
| (A-10) | 1-10 | CDR-H1 | 14 | EYTIH |
| | | CDR-H2 | 43 | GINPNNGNTRYDQKFKG |
| | | CDR-H3 | 44 | VARSSGSGPYAMDY |
| | | CDR-L1 | 45 | RCTQSLLHTNGDTYLH |
| | | CDR-L2 | 18 | KVSNRFS |
| | | CDR-L3 | 19 | SQSTHVPYT |
| (A-11) | 1-11 | CDR-H1 | 46 | GYNMH |
| | | CDR-H2 | 47 | AISPGKGDTSYNLKFKG |
| | | CDR-H3 | 48 | SGGTPFAY |
| | | CDR-L1 | 49 | RSSQSLVHSNGDTYLY |
| | | CDR-L2 | 18 | KVSNRFS |
| | | CDR-L3 | 19 | SQSTHVPYT |
| (A-12) | 1-12 | CDR-H1 | 50 | ANAMN |
| | | CDR-H2 | 7 | RIRSKSNNYATYYADSVKD |
| | | CDR-H3 | 51 | GSDNYIFYAMDY |
| | | CDR-L1 | 9 | RSSKSLLHSNGNTYLY |
| | | CDR-L2 | 10 | RMSNLAS |
| | | CDR-L3 | 11 | MQHLEYPFT |
| (A-13) | 1-13 | CDR-H1 | 52 | TNAMN |
| | | CDR-H2 | 53 | RIRSKSNYYATYYADSVKD |
| | | CDR-H3 | 54 | GREMGNYYSMDY |
| | | CDR-L1 | 9 | RSSKSLLHSNGNTYLY |
| | | CDR-L2 | 10 | RMSNLAS |
| | | CDR-L3 | 11 | MQHLEYPFT |
| (A-14) | 1-14 | CDR-H1 | 33 | NYRMH |
| | | CDR-H2 | 34 | VIKVKSDNYGANYAESVKG |
| | | CDR-H3 | 55 | PTYPGSSGFAY |
| | | CDR-L1 | 56 | RASQDISNYLN |
| | | CDR-L2 | 57 | YTSRLHS |
| | | CDR-L3 | 58 | QQGNKFPPT |
| (A-15) | 1-15 | CDR-H1 | 59 | EYTMH |
| | | CDR-H2 | 60 | GINPNNGDTNYNQKFMG |
| | | CDR-H3 | 61 | RLLRRGAMDY |
| | | CDR-L1 | 62 | RASENVEYYGTSLMQ |
| | | CDR-L2 | 63 | AASNVDS |
| | | CDR-L3 | 64 | QQSRKVPWT |
| (A-16) | 1-16 | CDR-H1 | 65 | GSYMH |
| | | CDR-H2 | 66 | RINPYNGATSYNQNFKD |
| | | CDR-H3 | 67 | TLLRLLDY |
| | | CDR-L1 | 68 | RSSKSLLHSNGITYLY |
| | | CDR-L2 | 69 | QMSNLAS |
| | | CDR-L3 | 70 | AQNLELPWT |
| (A-17) | 1-17 | CDR-H1 | 71 | SYWMH |
| | | CDR-H2 | 72 | NIWPGSASTNYDEKFKN |
| | | CDR-H3 | 73 | GGKGAMDY |
| | | CDR-L1 | 74 | RSSQSLENSYGNTYLN |
| | | CDR-L2 | 75 | RVSNRFS |
| | | CDR-L3 | 76 | LQVTHVPPT |
| (A-18) | 1-18 | CDR-H1 | 77 | TYAMH |
| | | CDR-H2 | 78 | HINPSSGYSNYNQKFKD |
| | | CDR-H3 | 79 | SEVRRGYFDV |
| | | CDR-L1 | 80 | RTSQDIRNYLN |
| | | CDR-L2 | 57 | YTSRLHS |
| | | CDR-L3 | 81 | QQGNTLPPT |
| (A-19) | 1-19 | CDR-H1 | 122 | GFTFNTYA |
| | | CDR-H2 | 123 | IRSKSNNYAT |
| | | CDR-H3 | 124 | VRGREAYYRYDGGYYAMDV |
| | | CDR-L1 | 125 | KSLLHSNGNTY |
| | | CDR-L2 | — | RMS |
| | | CDR-L3 | 11 | MQHLEYPFT |
| (A-20) | 1-20, 1-21, 1-26 | CDR-H1 | 126 | GFTFNAYA |
| | | CDR-H2 | 127 | IRSKSNDYAT |
| | | CDR-H3 | 124 | VRGREAYYRYDGGYYAMDV |
| | | CDR-L1 | 125 | KSLLHSNGNTY |
| | | CDR-L2 | — | RMS |
| | | CDR-L3 | 23 | MQHREYPFT |
| (A-21) | 1-22 | CDR-H1 | 126 | GFTFNAYA |
| | | CDR-H2 | 127 | IRSKSNDYAT |
| | | CDR-H3 | 124 | VRGREAYYRYDGGYYAMDV |
| | | CDR-L1 | 125 | KSLLHSNGNTY |
| | | CDR-L2 | — | RVS |
| | | CDR-L3 | 23 | MQHREYPFT |
| (A-22) | 1-23, 1-24, 1-33, | CDR-H1 | 128 | GYTFTEYT |
| | 1-34, 1-35, 1-36, | CDR-H2 | 129 | INPNNGNT |
| | 1-37, 1-38 | CDR-H3 | 130 | ARVARSSGSGPYAMDY |
| | | CDR-L1 | 131 | QSLLHTNGDTY |
| | | CDR-L2 | — | KVS |
| | | CDR-L3 | 19 | SQSTHVPYT |
| (A-23) | 1-27, 1-28, 1-29, | CDR-H1 | 126 | GFTFNAYA |
| | 1-30, 1-31, 1-32 | CDR-H2 | 127 | IRSKSNDYAT |
| | | CDR-H3 | 133 | VRGREAYYRYDGGYYAMDY |
| | | CDR-L1 | 125 | KSLLHSNGNTY |
| | | CDR-L2 | — | RMS |
| | | CDR-L3 | 23 | MQHREYPFT |
| (A-24) | 1-39, 1-40, 1-41, | CDR-H1 | 122 | GFTFNTYA |
| | 1-42, 1-43, 1-44 | CDR-H2 | 123 | IRSKSNNYAT |
| | | CDR-H3 | 133 | VRGREAYYRYDGGYYAMDY |
| | | CDR-L1 | 125 | KSLLHSNGNTY |
| | | CDR-L2 | — | RMS |
| | | CDR-L3 | 11 | MQHLEYPFT |
| (A-25) | 1-25, 1-45, 1-46, | CDR-H1 | 134 | GFTFSNYR |
| | 1-47, 1-48, 1-49, | CDR-H2 | 135 | IKVKSDNYGA |
| | 1-50 | CDR-H3 | 136 | SSPTYPGSSGFAY |
| | | CDR-L1 | 137 | QDISNY |
| | | CDR-L2 | — | YTS |
| | | CDR-L3 | 58 | QQGNKFPPT |

As is known in the art, deviation from the specific CDR sequences of an exemplary antibody is possible while still retaining the functionality and the specific binding exhibited by the corresponding exemplary antibody, e.g., as in standard humanization protocols. Such variants that have one or more amino acid substitutions/replacements in the CDRs and maintain the desired functional properties (as generally described herein in relation to the antibodies according to the invention) can be readily identified using routine techniques known in the art.

Accordingly, in a further embodiment, the antibody (or antigen-binding fragment) according to the invention comprises a VH domain comprising a CDR-H1, CDR-H2 and CDR-H3 as well as a VL domain comprising a CDR-L1, CDR-L2 and CDR-L3, as defined in any one of the above-described options (A-1) to (A-25), wherein a single amino acid residue in each one of these CDRs (preferably in one, two or three of these CDRs; more preferably in one or two of the CDRs; even more preferably in only one of the CDRs) is optionally substituted by another amino acid residue (i.e., is optionally replaced by a different amino acid residue). The resulting variant is an antibody (or antigen-binding fragment) according to the invention, i.e., a monoclonal antibody (or an antigen-binding fragment thereof) which specifically binds to human CCR8 and is an antagonist (preferably an insurmountable antagonist) of the CCL1-CCR8 signaling pathway. Thus, in this embodiment, the antibody (or antigen-binding fragment) according to the invention comprises a VH domain comprising a CDR-H1, CDR-H2 and CDR-H3 as well as a VL domain comprising a CDR-L1, CDR-L2 and CDR-L3, as defined in any one of the above-described options (A-1) to (A-25), wherein in each of said CDR-H1, said CDR-H2, said CDR-H3, said CDR-L1, said CDR-L2, and said CDR-L3, a single amino acid residue is optionally substituted (i.e., replaced) by a different amino acid residue; preferably wherein in one, two or three CDRs selected from said CDR-H1, said CDR-H2, said CDR-H3, said CDR-L1, said CDR-L2, and said CDR-L3, a single amino acid residue is optionally substituted (i.e., replaced) by a different amino acid residue; more preferably wherein in one or two CDRs selected from said CDR-H1, said CDR-H2, said CDR-H3, said CDR-L1, said CDR-L2, and said CDR-L3, a single amino acid residue is optionally substituted (i.e., replaced) by a different amino acid residue.

It is preferred that any such single amino acid substitution is a conservative amino acid substitution (i.e., a substitution/replacement with another amino acid that has similar physicochemical properties as the original amino acid), more preferably a highly conservative amino acid substitution (i.e., a substitution/replacement with another amino acid that has highly similar physicochemical properties as the original amino acid). Accordingly, whenever a single amino acid residue in any CDR is optionally substituted (i.e., replaced) by another amino acid residue, it is preferred that said other amino acid residue is selected using the following conservative amino acid substitution rules, more preferably using the following highly conservative amino acid substitution rules:

Conservative amino acid substitution rules

| Original amino acid | Substitution/replacement amino acid |
|---|---|
| glycine (Gly) | Ala, Val, Leu, Ile, Phe, Tyr, Trp, or Met |
| alanine (Ala) | Gly, Val, Leu, Ile, Phe, Tyr, Trp, or Met |
| valine (Val) | Gly, Ala, Leu, Ile, Phe, Tyr, Trp, or Met |
| leucine (Leu) | Gly, Ala, Val, Ile, Phe, Tyr, Trp, or Met |
| isoleucine (Ile) | Gly, Ala, Val, Leu, Phe, Tyr, Trp, or Met |
| phenylalanine (Phe) | Gly, Ala, Val, Leu, Ile, Tyr, Trp, or Met |
| tyrosine (Tyr) | Gly, Ala, Val, Leu, Ile, Phe, Trp, or Met |
| tryptophan (Trp) | Gly, Ala, Val, Leu, Ile, Phe, Tyr, or Met |
| methionine (Met) | Gly, Ala, Val, Leu, Ile, Phe, Tyr, or Trp |
| serine (Ser) | Thr, Asn, or Gln |
| threonine (Thr) | Ser, Asn, or Gln |
| asparagine (Asn) | Ser, Thr, or Gln |
| glutamine (Gln) | Ser, Thr, or Asn |
| arginine (Arg) | Lys or His |
| lysine (Lys) | Arg or His |
| histidine (His) | Arg or Lys |
| aspartic acid (Asp) | Glu |
| glutamic acid (Glu) | Asp |
| cysteine (Cys) | Ser or Ala |
| proline (Pro) | Ala |

Highly conservative amino acid substitution rules

| Original amino acid | Substitution/replacement amino acid |
|---|---|
| glycine (Gly) | Ala |
| alanine (Ala) | Gly, Val, Leu, or Ile |
| valine (Val) | Ala, Leu, or Ile |
| leucine (Leu) | Ala, Val, or Ile |
| isoleucine (Ile) | Ala, Val, or Leu |
| phenylalanine (Phe) | Tyr or Trp |
| tyrosine (Tyr) | Phe or Trp |
| tryptophan (Trp) | Phe or Tyr |
| methionine (Met) | Val, Leu, or Ile |
| serine (Ser) | Thr |
| threonine (Thr) | Ser |
| asparagine (Asn) | Gln |
| glutamine (Gln) | Asn |
| arginine (Arg) | Lys |
| lysine (Lys) | Arg |
| histidine (His) | Arg |
| aspartic acid (Asp) | Glu |
| glutamic acid (Glu) | Asp |
| cysteine (Cys) | Ser |
| proline (Pro) | Ala |

Moreover, in preferred embodiments, the antibody (or antigen-binding fragment) according to the invention comprises a heavy chain variable domain (VH) and/or a light chain variable domain (VL), wherein said VH domain and said VL domain each have an amino acid sequence with at least 80% (more preferably at least 85%, even more preferably at least 90%, even more preferably at least 92%, even more preferably at least 95%, yet even more preferably at least 97%, still more preferably 100%) sequence identity to the VH domain and the VL domain, respectively, of any one of the exemplary antibodies described in the examples section herein below. In particular, it is preferred that the antibody (or antigen-binding fragment) according to the invention (which, as explained above, specifically binds to human CCR8 and is an antagonist, particularly an insurmountable antagonist, of the CCL1-CCR8 signaling pathway) comprises:
(B-1) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MLLGLKWVFFVVFYQGVLCEVQLLESGGGLVQPKGSLKLSCAASGFTFNPYAMNWVRQAPGRGLEWVA RIRSKSNNYATYYADSVKDRFTISRDDSQDMLYLQMNNLKTEDTAMYYCVRGREAYYRYDGDYYAMDYW GQGTSVTVSS (SEQ ID NO: 82); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MRCLAEFLGLLVLWIPGAIGDIVMTQAAPSVPVTPGESVSISCRSSKSLLHSNGNTYLYWFLQRPGQSPQL LIYRMSNLASGVPDRFSGSGSGTAFTLRISRVEAEDVGVYYCMQHLEYPFTFGSGTKLEIK (SEQ ID NO: 83); or
(B-2) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MLLGLKWVFFVVFYQGVHCEVQLVESGGGLVQPKGSLKLSCAASGFTFNTYAMNWVRQAPGKGLEWVA RIRSKSNNYATYYADSVKDRFTISRDDSQSMLYLQMNNLKTEDTAMYYCVRGREAYYRYDGGYYAMDYW GQGTSVTVSS (SEQ ID NO: 84); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MRCLAEFLGLLVLWIPGAIGDIVMTQAAPSVPVTPGESVSISCRSSKSLLHSNGNTYLYWFLQRPGQSPQL LIYRMSNLASGVPDRFSGSGSGTAFTLRISRVEAEDVGVYYCMQHLEYPFTFGSGTKLEIK (SEQ ID NO: 83); or
(B-3) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MGWSWIFLFLLSGTAGVLSKVQLQQSGPELVKPGASVKISCKTSGYTFTEYTIHWVQQIHGKSPEWIGGIN PNGDTRYDQKFKGKATLTIDKSSSTAYMELRSLTSEDSAVYYCARVARFYGISPYAMDYWGQGTSVTVSS (SEQ ID NO: 85); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MKLPVRLLVLMFWIPASSSDVVMTQTPLSLPVSLGDQASISCRCTQSLLHSNGDTYLHWYLQKPGQSPNL LIYKVSNRFSGVPDRFSGSGSGTDFTLKINRVEAEDLGVYFCSQSTHVPYTFGGGTNLEIK (SEQ ID NO: 86); or
(B-4) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MLLGLKWVFFVVFYQGVHCEVQLVESGGGLVQPTGSLRLSCAASGFTFNAYAMNWVRQAPGKGLEWVA RIRSKSNDYATYYGDSVKDRFTISRDDSQSMLYLQMNNLKTEDTAMYYCVRGREAYYRYDGGYYAMDYW GQGTSVTVSS (SEQ ID NO: 87); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MRCLAEFLGLLVLWIPGAIGDIVMTQAAPSVSVTPGESVSISCTSSKSLLHSNGNTYLYWFLQRPGQSPQL LIYRMSNLASGVPDRFSGSGSGTAFTLRISRVEAEDVGVYYCMQHREYPFTFGSGTKLEIK (SEQ ID NO: 88); or
(B-5) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MAVLGLLLCLVTFPSCVLSQVQLKESGPGLVAPSQSLSITCTVSGLSMNDYVVTWIRQPPGKGLEWLGVIW GGGNTYYNSDLKSRLSITKDNSKSQVFFKMSSLQTDDTAVYYCARRHRDYALDYWGQGISVTVSS (SEQ ID NO: 89); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MKLPVRLLVLMFWIPASSSDVVMTQTPLSLPVSLGDQASISCRSSQSLVYSNGNTYLHWYLQKPGQSPKL LIYKVSNRFSGVPDRFSGSGSGTDFTLKISRVEAEDLGVYFCSQSTYVPPTFGGGTKLEIK (SEQ ID NO: 90); or
(B-6) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MGWSWIFLFLLSGTAGVLSEVQLQQSGPELLKPGTSVKISCTTSGYTFSDYTLHWVKQSHGKSLEWIGGIT PKNGDTRYDPRFKDKATLTIDKSSSAAYMELRSLTSEDSAVYYCARVARFYGVSPYAMDYWGQGASVTV SS (SEQ ID NO: 91); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MKLPVRLLVLMFWIPVSNSDVVMTQTPLSLPVSLGDQASISCRCTQSLLHSNGDTYLHWYLQKPGQSPKL LIYKVSNRFSGVPDRFSGSGSGTDFTLEITRVEAEDLGVYFCSQTTHVPYTFGGGTKLEIK (SEQ ID NO: 92); or
(B-7) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MELGLSWVFLVALLNGVQCQVHLVETGGGLVRPGNSLKLSCVTSGFTLSNYRMHWLRQPPGKRLEWIAVI KVKSDNYGANYAESVKGRFTISRDDAKSSVYLQMNRLREEDTATYYCSRPDGFSPFVYWGQGTLVTVSA (SEQ ID NO: 93); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MDMRTPAQFLGILLLWFPGIKCDIKMTQSPSSMYASLGERVTITCKASQDINSYLSWFQQKPGKSPKTLIYR ANRLVDGVPSRFSGSGSGQDYFLTISSLEYEDMGIYYCLQYGEFPPTFGAGTKLELK (SEQ ID NO: 94); or
(B-8) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MLLGLKWVFFVVFYQGVHCEVQLVESGGGLVQPKGSLRLSCAASGFTFNAYAMNWVRQAPGKGLEWVA RIRSKSNNYATYYGDSVKDRFTISRDDSQTMLYLQMNNLKTEDTAMYYCVRGREAYYRYDGDYYAMDYW GQGTSVTVSS (SEQ ID NO: 95); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MESQTQVLMFLLLWVSGACADIVMTQSPSSLAMSVGQKVTMSCKSSQSLLNSSNQKNYLAWYQQKPGQ SPKLLVYFASTRESGVPDRFIGSGSGTDFTLTISSVQAEDLADYFCQQHYSTPYTFGGGTKLEIK (SEQ ID NO: 96); or
(B-9) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MLLGLKWVFFVVFYQGVHCEVQLVESGGGLVQPKGSLKLSCAASGFTFNAYAMNWVRQAPGKGLEWLA RIRSKSNNYATYYADSVKDRFTISRDDSQSMFYLQMNNLKSEDTAMYYCVRGREAYYRYDGGYYAMDYW GQGTSVTVSS (SEQ ID NO: 97); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MRCLAEFLGLLVLWIPGAIGEIVMTQAALSAPVTPGESVSMSCRSSKSLLHSNGNTYLYWFLQRPGQSPQL LIYRMSNLASGVPDRFSGSGSGTAFTLRISRVEAEDVGVYYCMQHLEYPFTFGSGTKLEIK (SEQ ID NO: 98); or
(B-10) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MGWSWIFLFLLSGTAGVLSEVQLQQSGPELVKPGASVKISCKTSGYTFTEYTIHWVKQSHGESLEWVGGI NPNNGNTRYDQKFKGKATLTIDKSSSPAYMELRSLTSEDSAVYYCARVARSSGSGPYAMDYWGQGTSVT VSS (SEQ ID NO: 99); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MKLPVRLLVLMFWIPVSSSDVVMTQTPLSLPVSLGDQASISCRCTQSLLHTNGDTYLHWYLQKPGQSPKLL IYKVSNRFSGVPDRFSGSGSGTDFTLKISRVEAEDLGVYFCSQSTHVPYTFGGGTKLEIK (SEQ ID NO: 100); or
(B-11) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MGWSCIILFLVATATDVHSQVQLQQPGAELVKPGASVKMSCKATGYTFTGYNMHWVKQTPGQGLEWIGA ISPGKGDTSYNLKFKGKATLTTDKSSSTAYMQLSSLTSADSAVYYCARSGGTPFAYWGQGTLVTVSA (SEQ ID NO: 101); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MKLPVRLLVLMFWIPASSSDVVMTQTPLSLPVSLGDQASISCRSSQSLVHSNGDTYLYWYLQKPGQSPKL LIYKVSNRFSGVPDRFSGSGSGTDFTLKINRVEAEDLGVYFCSQSTHVPYTFGGGTKLEIK (SEQ ID NO: 102); or
(B-12) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MLLGLKWVFFVVFYQGVHCEVQLVETGGGLVQPKGSLKLSCAASGFTFNANAMNWVRQAPGKGLEWVA RIRSKSNNYATYYADSVKDRFTISRDDSQSMLYLQMNNLKTEDTAMYYCVRGSDNYIFYAMDYWGQGTS VTVSS (SEQ ID NO: 103); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MRCLAEFLGLLVLWIPGAIGDIVMTQAAPSVPVTPGESVSISCRSSKSLLHSNGNTYLYWFLQRPGQSPQL LIYRMSNLASGVPDRFSGSGSGTAFTLRISRVEAEDVGVYYCMQHLEYPFTFGGGTKLEIK (SEQ ID NO: 104); or
(B-13) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MLLGLKWVFFVVFYQGVHCEVQLVETGGGLVQPKGSLKLSCAASGFTFNTNAMNWVRQAPGKGLEWVA RIRSKSNYYATYYADSVKDRFTISRDDSQSMLYLQMNNLKTEDTAMYYCVRGREMGNYYSMDYWGQGT SVTVSS (SEQ ID NO: 105); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MRCLAEFLGLLVLWIPGAIGDIVMTQAAPSVPVTPGESVSISCRSSKSLLHSNGNTYLYWFLQRPGQSPQL LIYRMSNLASGVPDRFSGSGSGTAFTLRISRVEAEDVGVYYCMQHLEYPFTFGSGTKLEIK (SEQ ID NO: 83); or
(B-14) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MELGLSWVFLVALLNGVQCQVQLVETGGGLVRPGNSLKLSCVTSGFTFSNYRMHWLRQPPGKRLEWIAV IKVKSDNYGANYAESVKGRFTISRDDSKSSVYLQVNRLREEDTATYYCSSPTYPGSSGFAYWGQGTLVTV SA (SEQ ID NO: 106); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MMSSAQFLGLLLLCFQGTRCDIQMTQTTSSLSASLGDRVTISCRASQDISNYLNWYQQKPDGTVKLLIYYT SRLHSGVPSRFSGSGSGTDYSLTISNLEQEDIATYFCQQGNKFPPTFGGGTKLEIN (SEQ ID NO: 107); or
(B-15) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MGWSWIFLFLLSGTAGVLSEVQLQQSGPELVKPGASVKISCKTSGYTFTEYTMHWVKQSHGKSLEWIGGI NPNNGDTNYNQKFMGKATLTVDKSSSTAYMELRSLTSEDSAVYYCVRRLLRRGAMDYWGQGTSVTVSS (SEQ ID NO: 108); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MESDTLLLWVLLLWVPGSTGDIVLTQSPASLAVSLGQRATISCRASENVEYYGTSLMQWFQQKPGQPPKL LIYAASNVDSGVPARFSGSGSGTDFSLNIHPVEEDDIAMYFCQQSRKVPWTFGGGTKLDIK (SEQ ID NO: 109); or
(B-16) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MGWSWIFLFLLSGTAGVLSEVQLQQSGPELVKPGASVKISCKASVYSFTGSYMHWVKQSHVKSLEWIGRI NPYNGATSYNQNFKDKASLTVDKSSSTAYMELHSLTSEDSAVYYCATTLLRLLDYWGQGTTLTVSS (SEQ ID NO: 110); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MRFSAQLLGLLVLWIPGSTADIVMTQAAFSNPVTLGTSASISCRSSKSLLHSNGITYLYWYLQKPGQSPQLL IYQMSNLASGVPDRESSSGSGTDFTLRISRVEAEDVGVYYCAQNLELPWTFGGGTKLEIK (SEQ ID NO: 111); or
(B-17) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MGWSSIILFLVATASGVHSQVQLQQPGSELVRPGASVKLSCKASGYTFTSYWMHWVKQRPGQGLEWIGN IWPGSASTNYDEKFKNKATLTVDTSSSTAYMQLSSLTSEDSAVYYCIRGGKGAMDYWSQGTSVTVSS (SEQ ID NO: 112); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MKLPVRLLVLMFWIPVSSSDVVMTQIPLSLPVSLGDQASISCRSSQSLENSYGNTYLNWYLQKPGQSPQLL IYRVSNRFSGVLDRFSGSGSGTDFTLKISRVEAEDLGVYFCLQVTHVPPTFGAGTKLELK (SEQ ID NO: 113); or
(B-18) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MERHWIFLLLLSVTAGVHSQVQVQQSGAELARPGASVKMSCKASGYTFTTYAMHWVKQRPGQGLEWIG HINPSSGYSNYNQKFKDKATLTADKSSSTAYMQLSSLTSEDSAVYYCARSEVRRGYFDVWGAGTTVTVSS (SEQ ID NO: 114); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence MMSSAQFLGLLLLCFQGTRCDIQMTQTTSSLSASLGDRVTISCRTSQDIRNYLNWYQQKPDGTVKLLISYT SRLHSGVPSRFSGSGSGTDYSLTISNLEQEDFATYFCQQGNTLPPTFGGGTRVEIK (SEQ ID NO: 115); or
(B-19) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence EVOLVESGGGLVQPGRSLRLSCTASGFTFNTYAMNWVRQAPGKGLEWVARIRSKSNNYATYYAASVKGR FTISRDDSKSIAYLQMNSLKTEDTAVYYCVRGREAYYRYDGGYYAMDVWGQGTTVTVSS (SEQ ID NO: 138); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence DIVMTQAAPSLPVTPGESASISCRSSKSLLHSNGNTYLYWFLQKPGQSPQLLIYRMSNLASGVPDRFSGSG SGTAFTLKISRVEAEDVGVYYCMQHLEYPFTFGSGTKLEIK (SEQ ID NO: 139); or
(B-20) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence EVQLLESGGGLVQPGGSLRLSCAASGFTFNAYAMNWVRQAPGKGLEWVARIRSKSNDYATYYGDSVKG RFTISRDNSKNTLYLQMNSLRAEDTAVYYCVRGREAYYRYDGGYYAMDVWGQGTTVTVSS (SEQ ID NO: 140); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence DIVMTQAAPSLSVTPGESASISCTSSKSLLHSNGNTYLYWFLQKPGQSPQLLIYRMSNLASGVPDRFSGSG SGTAFTLKISRVEAEDVGVYYCMQHREYPFTFGSGTKLEIK (SEQ ID NO: 141); or
(B-21) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence EVQLLESGGGLVQPGGSLRLSCAASGFTFNAYAMNWVRQAPGKGLEWVARIRSKSNDYATYYGDSVKG RFTISRDNSKNTLYLQMNSLRAEDTAVYYCVRGREAYYRYDGGYYAMDVWGQGTTVTVSS (SEQ ID NO: 140); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence DIVMTQSPLSLPVTPGEPASISCRSSKSLLHSNGNTYLYWFLQKPGQSPQLLIYRVSNLASGVPDRFSGSG SGTDFTLKISRVEAEDVGVYYCMQHREYPFTFGQGTKLEIK (SEQ ID NO: 142); or
(B-22) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence QVQLVQSGPEVVKPGASVKVSCKTSGYTFTEYTIHWVRQAPGQSLEWVGGINPNNGNTRYDQKFQGRV TITRDKSASTAYMELRSLTSEDTAVYYCARVARSSGSGPYAMDYWGQGTTVTVSS (SEQ ID NO: 143); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence DVVMTQSPLSLPVTLGDQASISCRCSQSLLHTNGDTYLHWYLQRPGQSPRLLIYKVSNRFSGVPDRFSGS GSGTDFTLKISRVEAEDVGVYFCSQSTHVPYTFGGGTKVEIK (SEQ ID NO: 144); or
(B-23) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence QVQLQQSGPEVVKPGASVKVSCKTSGYTFTEYTIHWVRQSHGQSLEWVGGINPNNGNTRYDQKFKGRV TITIDKSSSTAYMELRSLTSEDTAVYYCARVARSSGSGPYAMDYWGQGTTVTVSS (SEQ ID NO: 145); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence DVVMTQSPLSLPVTLGDQASISCRCSQSLLHTNGDTYLHWYLQRPGQSPRLLIYKVSNRFSGVPDRFSGS GSGTDFTLKISRVEAEDVGVYFCSQSTHVPYTFGGGTKVEIK (SEQ ID NO: 144); or
(B-24) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence QVQLVESGGGVVQPGRSLRLSCAASGFTFSNYRMHWVRQAPGKGLEWIAVIKVKSDNYGANYADSVKG RFTISRDNSKNTLYLQMNSLRAEDTAVYYCSSPTYPGSSGFAYWGQGTLVTVSS (SEQ ID NO: 146); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence DIQMTQSPSSLSASVGDRVTITCRASQDISNYLNWYQQKPDGTVKLLIYYTSRLHTGVPSRFSGSGSGTDY TLTISNLQQEDIATYFCQQGNKFPPTFGGGTKVEIN (SEQ ID NO: 132); or
(B-25) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence EVOLVESGGGLVQPTGSLRLSCAASGFTFNAYAMNWVRQAPGKGLEWVARIRSKSNDYATYYGDSVKD RFTISRDDSQSMLYLQMNNLKTEDTAMYYCVRGREAYYRYDGGYYAMDYWGQGTSVTVSS (SEQ ID NO: 147); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence DIVMTQAAPSVSVTPGESVSISCTSSKSLLHSNGNTYLYWFLQRPGQSPQLLIYRMSNLASGVPDRFSGS GSGTAFTLRISRVEAEDVGVYYCMQHREYPFTFGSGTKLEIK (SEQ ID NO: 148); or
(B-26) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence EVQLQQSGPELVKPGASVKISCKTSGYTFTEYTIHWVKQSHGESLEWVGGINPNNGNTRYDQKFKGKATL TIDKSSSPAYMELRSLTSEDSAVYYCARVARSSGSGPYAMDYWGQGTSVTVSS (SEQ ID NO: 149); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence DVVMTQTPLSLPVSLGDQASISCRCTQSLLHTNGDTYLHWYLQKPGQSPKLLIYKVSNRFSGVPDRFSGS GSGTDFTLKISRVEAEDLGVYFCSQSTHVPYTFGGGTKLEIK (SEQ ID NO: 150); or
(B-27) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence EVOLVESGGGLVQPKGSLKLSCAASGFTFNTYAMNWVRQAPGKGLEWVARIRSKSNNYATYYADSVKDR FTISRDDSQSMLYLQMNNLKTEDTAMYYCVRGREAYYRYDGGYYAMDYWGQGTSVTVSS (SEQ ID NO: 151); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence DIVMTQAAPSVPVTPGESVSISCRSSKSLLHSNGNTYLYWFLQRPGQSPQLLIYRMSNLASGVPDRFSGS GSGTAFTLRISRVEAEDVGVYYCMQHLEYPFTFGSGTKLEIK (SEQ ID NO: 152); or
(B-28) a heavy chain variable domain (VH) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence QVQLVETGGGLVRPGNSLKLSCVTSGFTFSNYRMHWLRQPPGKRLEWIAVIKVKSDNYGANYAESVKGR FTISRDDSKSSVYLQVNRLREEDTATYYCSSPTYPGSSGFAYWGQGTLVTVSA (SEQ ID NO: 153); and/or (preferably; and)
   a light chain variable domain (VL) having an amino acid sequence with at least 80% (more preferably at least 85%, even more preferably at least 90%, even more preferably at least 92%, even more preferably at least 95%, yet even more preferably at least 97%, still more preferably 100%) sequence identity to the amino acid sequence DIQMTQTTSSLSASLGDRVTISCRASQDISNYLNWYQQKPDGTVKLLIYYTSRLHSGVPSRFSGSGSGTDY SLTISNLEQEDIATYFCQQGNKFPPTFGGGTKLEIN (SEQ ID NO: 154).

For each of the above-described options (B-1) to (B-28), the VH domain and the VL domain are each defined by a percent sequence identity to a certain reference sequence, and preferred values (lower endpoints) are indicated for the percent sequence identity in each case. While the sequence identity for any particular domain can, in principle, be selected independently from the sequence identity for any other domain, it is generally preferred that the same percent values (lower endpoints) are selected for the sequence identity of the VH domain and for the sequence identity of the VL domain of the same antibody (or antigen-binding fragment). Thus, for example, if in option (B-1) the VH domain is chosen to have an amino acid sequence with “at least 90%” sequence identity to SEQ ID NO: 82, then it is preferred to choose the same percent sequence identity for the VL domain, i.e., to choose the VL domain as having an amino acid sequence with “at least 90%” sequence identity to SEQ ID NO: 83. This analogously applies to the percent sequence identities of any other pairs of VH and VL domains disclosed herein.

The above-mentioned VH and VL domain sequences are also summarized in the following table:

| | | | SEQ | |
|---|---|---|---|---|
| | | Variable | ID | |
| | Antibody | domain | NO: | Aminoa cid sequence |
| (B-1) | 1-1 | VH | 82 | MLLGLKWVFFVVFYQGVLCEVQLLESGGGLVQPKGSLKLS |
| | | | | CAASGFTFNPYAMNWVRQAPGRGLEWVARIRSKSNNYAT |
| | | | | YYADSVKDRFTISRDDSQDMLYLQMNNLKTEDTAMYYCVR |
| | | | | GREAYYRYDGDYYAMDYWGQGTSVTVSS |
| | | VL | 83 | MRCLAEFLGLLVLWIPGAIGDIVMTQAAPSVPVTPGESVSIS |
| | | | | CRSSKSLLHSNGNTYLYWFLQRPGQSPQLLIYRMSNLASG |
| | | | | VPDRFSGSGSGTAFTLRISRVEAEDVGVYYCMQHLEYPFT |
| | | | | FGSGTKLEIK |
| (B-2) | 1-2 | VH | 84 | MLLGLKWVFFVVFYQGVHCEVQLVESGGGLVQPKGSLKLS |
| | | | | CAASGFTFNTYAMNWVRQAPGKGLEWVARIRSKSNNYAT |
| | | | | YYADSVKDRFTISRDDSQSMLYLQMNNLKTEDTAMYYCVR |
| | | | | GREAYYRYDGGYYAMDYWGQGTSVTVSS |
| | | VL | 83 | MRCLAEFLGLLVLWIPGAIGDIVMTQAAPSVPVTPGESVSIS |
| | | | | CRSSKSLLHSNGNTYLYWFLQRPGQSPQLLIYRMSNLASG |
| | | | | VPDRFSGSGSGTAFTLRISRVEAEDVGVYYCMQHLEYPFT |
| | | | | FGSGTKLEIK |
| (B-3) | 1-3 | VH | 85 | MGWSWIFLFLLSGTAGVLSKVQLQQSGPELVKPGASVKIS |
| | | | | CKTSGYTFTEYTIHWVQQIHGKSPEWIGGINPNGDTRYDQK |
| | | | | FKGKATLTIDKSSSTAYMELRSLTSEDSAVYYCARVARFYGI |
| | | | | SPYAMDYWGQGTSVTVSS |
| | | VL | 86 | MKLPVRLLVLMFWIPASSSDWVMTQTPLSLPVSLGDQASIS |
| | | | | CRCTQSLLHSNGDTYLHWYLQKPGQSPNLLIYKVSNRFSG |
| | | | | VPDRFSGSGSGTDFTLKINRVEAEDLGVYFCSQSTHVPYTF |
| | | | | GGGTNLEIK |
| (B-4) | 1-4 | VH | 87 | MLLGLKWVFFVVFYQGVHCEVQLVESGGGLVQPTGSLRLS |
| | | | | CAASGFTFNAYAMNWVRQAPGKGLEWVARIRSKSNDYAT |
| | | | | YYGDSVKDRFTISRDDSQSMLYLQMNNLKTEDTAMYYCVR |
| | | | | GREAYYRYDGGYYAMDYWGQGTSVTVSS |
| | | VL | 88 | MRCLAEFLGLLVLWIPGAIGDIVMTQAAPSVSVTPGESVSIS |
| | | | | CTSSKSLLHSNGNTYLYWFLQRPGQSPQLLIYRMSNLASG |
| | | | | VPDRFSGSGSGTAFTLRISRVEAEDVGVYYCMQHREYPFT |
| | | | | FGSGTKLEIK |
| (B-5) | 1-5 | VH | 89 | MAVLGLLLCLVTFPSCVLSQVQLKESGPGLVAPSQSLSITC |
| | | | | TVSGLSMNDYVVTWIRQPPGKGLEWLGVIWGGGNTYYNS |
| | | | | DLKSRLSITKDNSKSQVFFKMSSLQTDDTAVYYCARRHRDY |
| | | | | ALDYWGQGISVTVSS |
| | | VL | 90 | MKLPVRLLVLMFWIPASSSDWMTQTPLSLPVSLGDQASIS |
| | | | | CRSSQSLVYSNGNTYLHWYLQKPGQSPKLLIYKVSNRFSG |
| | | | | VPDRFSGSGSGTDFTLKISRVEAEDLGVYFCSQSTYVPPTF |
| | | | | GGGTKLEIK |
| (B-6) | 1-6 | VH | 91 | MGWSWIFLFLLSGTAGVLSEVQLQQSGPELLKPGTSVKISC |
| | | | | TTSGYTFSDYTLHWVKQSHGKSLEWIGGITPKNGDTRYDP |
| | | | | RFKDKATLTIDKSSSAAYMELRSLTSEDSAVYYCARVARFY |
| | | | | GVSPYAMDYWGQGASVTVSS |
| | | VL | 92 | MKLPVRLLVLMFWIPVSNSDVVMTQTPLSLPVSLGDQASIS |
| | | | | CRCTQSLLHSNGDTYLHWYLQKPGQSPKLLIYKVSNRFSG |
| | | | | VPDRFSGSGSGTDFTLEITRVEAEDLGVYFCSQTTHVPYTF |
| | | | | GGGTKLEIK |
| (B-7) | 1-7 | VH | 93 | MELGLSWVFLVALLNGVQCQVHLVETGGGLVRPGNSLKLS |
| | | | | CVTSGFTLSNYRMHWLRQPPGKRLEWIAVIKVKSDNYGAN |
| | | | | YAESVKGRFTISRDDAKSSVYLQMNRLREEDTATYYCSRP |
| | | | | DGFSPFVYWGQGTLVTVSA |
| | | VL | 94 | MDMRTPAQFLGILLLWFPGIKCDIKMTQSPSSMYASLGERV |
| | | | | TITCKASQDINSYLSWFQQKPGKSPKTLIYRANRLVDGVPS |
| | | | | RFSGSGSGQDYFLTISSLEYEDMGIYYCLQYGEFPPTFGAG |
| | | | | TKLELK |
| (B-8) | 1-8 | VH | 95 | MLLGLKWVFFVVFYQGVHCEVQLVESGGGLVQPKGSLRLS |
| | | | | CAASGFTFNAYAMNWVRQAPGKGLEWVARIRSKSNNYAT |
| | | | | YYGDSVKDRFTISRDDSQTMLYLQMNNLKTEDTAMYYCVR |
| | | | | GREAYYRYDGDYYAMDYWGQGTSVTVSS |
| | | VL | 96 | MESQTQVLMFLLLWVSGACADIVMTQSPSSLAMSVGQKVT |
| | | | | MSCKSSQSLLNSSNQKNYLAWYQQKPGQSPKLLVYFAST |
| | | | | RESGVPDRFIGSGSGTDFTLTISSVQAEDLADYFCQQHYST |
| | | | | PYTFGGGTKLEIK |
| (B-9) | 1-9 | VH | 97 | MLLGLKWVFFVVFYQGVHCEVQLVESGGGLVQPKGSLKLS |
| | | | | CAASGFTFNAYAMNWVRQAPGKGLEWLARIRSKSNNYAT |
| | | | | YYADSVKDRFTISRDDSQSMFYLQMNNLKSEDTAMYYCVR |
| | | | | GREAYYRYDGGYYAMDYWGQGTSVTVSS |
| | | VL | 98 | MRCLAEFLGLLVLWIPGAIGEIVMTQAALSAPVTPGESVSM |
| | | | | SCRSSKSLLHSNGNTYLYWFLQRPGQSPQLLIYRMSNLAS |
| | | | | GVPDRFSGSGSGTAFTLRISRVEAEDVGVYYCMQHLEYPF |
| | | | | TFGSGTKLEIK |
| (B-10) | 1-10 | VH | 99 | MGWSWIFLFLLSGTAGVLSEVQLQQSGPELVKPGASVKIS |
| | | | | CKTSGYTFTEYTIHWVKQSHGESLEWVGGINPNNGNTRYD |
| | | | | QKFKGKATLTIDKSSSPAYMELRSLTSEDSAVYYCARVARS |
| | | | | SGSGPYAMDYWGQGTSVTVSS |
| | | VL | 100 | MKLPVRLLVLMFWIPVSSSDWVMTQTPLSLPVSLGDQASIS |
| | | | | CRCTQSLLHTNGDTYLHWYLQKPGQSPKLLIYKVSNRFSG |
| | | | | VPDRFSGSGSGTDFTLKISRVEAEDLGVYFCSQSTHVPYTF |
| | | | | GGGTKLEIK |
| (B-11) | 1-11 | VH | 101 | MGWSCIILFLVATATDVHSQVQLQQPGAELVKPGASVKMS |
| | | | | CKATGYTFTGYNMHWVKQTPGQGLEWIGAISPGKGDTSY |
| | | | | NLKFKGKATLTTDKSSSTAYMQLSSLTSADSAVYYCARSG |
| | | | | GTPFAYWGQGTLVTVSA |
| | | VL | 102 | MKLPVRLLVLMFWIPASSSDWVMTQTPLSLPVSLGDQASIS |
| | | | | CRSSQSLVHSNGDTYLYWYLQKPGQSPKLLIYKVSNRFSG |
| | | | | VPDRFSGSGSGTDFTLKINRVEAEDLGVYFCSQSTHVPYTF |
| | | | | GGGTKLEIK |
| (B-12) | 1-12 | VH | 103 | MLLGLKWVFFWVFYQGVHCEVQLVETGGGLVQPKGSLKLS |
| | | | | CAASGFTFNANAMNWVRQAPGKGLEWVARIRSKSNNYAT |
| | | | | YYADSVKDRFTISRDDSQSMLYLQMNNLKTEDTAMYYCVR |
| | | | | GSDNYIFYAMDYWGQGTSVTVSS |
| | | VL | 104 | MRCLAEFLGLLVLWIPGAIGDIVMTQAAPSVPVTPGESVSIS |
| | | | | CRSSKSLLHSNGNTYLYWFLQRPGQSPQLLIYRMSNLASG |
| | | | | VPDRFSGSGSGTAFTLRISRVEAEDVGVYYCMQHLEYPFT |
| | | | | FGGGTKLEIK |
| (B-13) | 1-13 | VH | 105 | MLLGLKWVFFVVFYQGVHCEVQLVETGGGLVQPKGSLKLS |
| | | | | CAASGFTFNTNAMNWVRQAPGKGLEWVARIRSKSNYYAT |
| | | | | YYADSVKDRFTISRDDSQSMLYLQMNNLKTEDTAMYYCVR |
| | | | | GREMGNYYSMDYWGQGTSVTVSS |
| | | VL | 83 | MRCLAEFLGLLVLWIPGAIGDIVMTQAAPSVPVTPGESVSIS |
| | | | | CRSSKSLLHSNGNTYLYWFLQRPGQSPQLLIYRMSNLASG |
| | | | | VPDRFSGSGSGTAFTLRISRVEAEDVGVYYCMQHLEYPFT |
| | | | | FGSGTKLEIK |
| (B-14) | 1-14 | VH | 106 | MELGLSWVFLVALLNGVQCQVQLVETGGGLVRPGNSLKLS |
| | | | | CVTSGFTFSNYRMHWLRQPPGKRLEWIAVIKVKSDNYGAN |
| | | | | YAESVKGRFTISRDDSKSSVYLQVNRLREEDTATYYCSSPT |
| | | | | YPGSSGFAYWGQGTLVTVSA |
| | | VL | 107 | MMSSAQFLGLLLLCFQGTRCDIQMTQTTSSLSASLGDRVTI |
| | | | | SCRASQDISNYLNWYQQKPDGTVKLLIYYTSRLHSGVPSRF |
| | | | | SGSGSGTDYSLTISNLEQEDIATYFCQQGNKFPPTFGGGTK |
| | | | | LEIN |
| (B-15) | 1-15 | VH | 108 | MGWSWIFLFLLSGTAGVLSEVQLQQSGPELVKPGASVKIS |
| | | | | CKTSGYTFTEYTMHWVKQSHGKSLEWIGGINPNNGDTNYN |
| | | | | QKFMGKATLTVDKSSSTAYMELRSLTSEDSAVYYCVRRLL |
| | | | | RRGAMDYWGQGTSVTVSS |
| | | VL | 109 | MESDTLLLWVLLLWVPGSTGDIVLTQSPASLAVSLGQRATI |
| | | | | SCRASENVEYYGTSLMQWFQQKPGQPPKLLIYAASNVDSG |
| | | | | VPARFSGSGSGTDFSLNIHPVEEDDIAMYFCQQSRKVPWT |
| | | | | FGGGTKLDIK |
| (B-16) | 1-16 | VH | 110 | MGWSWIFLFLLSGTAGVLSEVQLQQSGPELVKPGASVKIS |
| | | | | CKASVYSFTGSYMHWVKQSHVKSLEWIGRINPYNGATSYN |
| | | | | QNFKDKASLTVDKSSSTAYMELHSLTSEDSAVYYCATTLLR |
| | | | | LLDYWGQGTTLTVSS |
| | | VL | 111 | MRFSAQLLGLLVLWIPGSTADIVMTQAAFSNPVTLGTSASIS |
| | | | | CRSSKSLLHSNGITYLYWYLQKPGQSPQLLIYQMSNLASGV |
| | | | | PDRFSSSGSGTDFTLRISRVEAEDVGVYYCAQNLELPWTF |
| | | | | GGGTKLEIK |
| (B-17) | 1-17 | VH | 112 | MGWSSIILFLVATASGVHSQVQLQQPGSELVRPGASVKLS |
| | | | | CKASGYTFTSYWMHWVKQRPGQGLEWIGNIWPGSASTNY |
| | | | | DEKFKNKATLTVDTSSSTAYMQLSSLTSEDSAVYYCIRGGK |
| | | | | GAMDYWSQGTSVTVSS |
| | | VL | 113 | MKLPVRLLVLMFWIPVSSSDWMTQIPLSLPVSLGDQASISC |
| | | | | RSSQSLENSYGNTYLNWYLQKPGQSPQLLIYRVSNRFSGV |
| | | | | LDRFSGSGSGTDFTLKISRVEAEDLGVYFCLQVTHVPPTFG |
| | | | | AGTKLELK |
| (B-18) | 1-18 | VH | 114 | MERHWIFLLLLSVTAGVHSQVQVQQSGAELARPGASVKMS |
| | | | | CKASGYTFTTYAMHWVKQRPGQGLEWIGHINPSSGYSNY |
| | | | | NQKFKDKATLTADKSSSTAYMQLSSLTSEDSAVYYCARSE |
| | | | | VRRGYFDVWGAGTTVTVSS |
| | | VL | 115 | MMSSAQFLGLLLLCFQGTRCDIQMTQTTSSLSASLGDRVTI |
| | | | | SCRTSQDIRNYLNWYQQKPDGTVKLLISYTSRLHSGVPSRF |
| | | | | SGSGSGTDYSLTISNLEQEDFATYFCQQGNTLPPTFGGGT |
| | | | | RVEIK |
| (B-19) | 1-19 | VH | 138 | EVQLVESGGGLVQPGRSLRLSCTASGFTFNTYAMNWVRQ |
| | | | | APGKGLEWVARIRSKSNNYATYYAASVKGRFTISRDDSKSI |
| | | | | AYLQMNSLKTEDTAVYYCVRGREAYYRYDGGYYAMDVWG |
| | | | | QGTTVTVSS |
| | | VL | 139 | DIVMTQAAPSLPVTPGESASISCRSSKSLLHSNGNTYLYWF |
| | | | | LQKPGQSPQLLIYRMSNLASGVPDRFSGSGSGTAFTLKISR |
| | | | | VEAEDVGVYYCMQHLEYPFTFGSGTKLEIK |
| (B-20) | 1-20, | VH | 140 | EVQLLESGGGLVQPGGSLRLSCAASGFTFNAYAMNWVRQ |
| | 1-21 | | | APGKGLEWVARIRSKSNDYATYYGDSVKGRFTISRDNSKN |
| | 1-26 | | | TLYLQMNSLRAEDTAVYYCVRGREAYYRYDGGYYAMDVW |
| | | | | GQGTTVTVSS |
| | | VL | 141 | DIVMTQAAPSLSVTPGESASISCTSSKSLLHSNGNTYLYWF |
| | | | | LQKPGQSPQLLIYRMSNLASGVPDRFSGSGSGTAFTLKISR |
| | | | | VEAEDVGVYYCMQHREYPFTFGSGTKLEIK |
| (B-21) | 1-22 | VH | 140 | EVQLLESGGGLVQPGGSLRLSCAASGFTFNAYAMNWVRQ |
| | | | | APGKGLEWVARIRSKSNDYATYYGDSVKGRFTISRDNSKN |
| | | | | TLYLQMNSLRAEDTAVYYCVRGREAYYRYDGGYYAMDVW |
| | | | | GQGTTVTVSS |
| | | VL | 142 | DIVMTQSPLSLPVTPGEPASISCRSSKSLLHSNGNTYLYWF |
| | | | | LQKPGQSPQLLIYRVSNLASGVPDRFSGSGSGTDFTLKISR |
| | | | | VEAEDVGVYYCMQHREYPFTFGQGTKLEIK |
| (B-22) | 1-23 | VH | 143 | QVQLVQSGPEVVKPGASVKVSCKTSGYTFTEYTIHWVRQA |
| | | | | PGQSLEWVGGINPNNGNTRYDQKFQGRVTITRDKSASTAY |
| | | | | MELRSLTSEDTAVYYCARVARSSGSGPYAMDYWGQGTTV |
| | | | | TVSS |
| | | VL | 144 | DWVMTQSPLSLPVTLGDQASISCRCSQSLLHTNGDTYLHW |
| | | | | YLQRPGQSPRLLIYKVSNRFSGVPDRFSGSGSGTDFTLKIS |
| | | | | RVEAEDVGVYFCSQSTHVPYTFGGGTKVEIK |
| (B-23) | 1-24 | VH | 145 | QVQLQQSGPEVVKPGASVKVSCKTSGYTFTEYTIHWVRQS |
| | | | | HGQSLEWVGGINPNNGNTRYDQKFKGRVTITIDKSSSTAY |
| | | | | MELRSLTSEDTAVYYCARVARSSGSGPYAMDYWGQGTTV |
| | | | | TVSS |
| | | VL | 144 | DVVMTQSPLSLPVTLGDQASISCRCSQSLLHTNGDTYLHW |
| | | | | YLQRPGQSPRLLIYKVSNRFSGVPDRFSGSGSGTDFTLKIS |
| | | | | RVEAEDVGVYFCSQSTHVPYTFGGGTKVEIK |
| (B-24) | 1-25 | VH | 146 | QVQLVESGGGVVQPGRSLRLSCAASGFTFSNYRMHWVRQ |
| | | | | APGKGLEWIAVIKVKSDNYGANYADSVKGRFTISRDNSKNT |
| | | | | LYLQMNSLRAEDTAVYYCSSPTYPGSSGFAYWGQGTLVTV |
| | | | | SS |
| | | VL | 132 | DIQMTQSPSSLSASVGDRVTITCRASQDISNYLNWYQQKP |
| | | | | DGTVKLLIYYTSRLHTGVPSRFSGSGSGTDYTLTISNLQQE |
| | | | | DIATYFCQQGNKFPPTFGGGTKVEIN |
| (B-25) | 1-27, | VH | 147 | EVQLVESGGGLVQPTGSLRLSCAASGFTFNAYAMNWVRQ |
| | 1-28, | | | APGKGLEWVARIRSKSNDYATYYGDSVKDRFTISRDDSQS |
| | 1-29, | | | MLYLQMNNLKTEDTAMYYCVRGREAYYRYDGGYYAMDY |
| | 1-30, | | | WGQGTSVTVSS |
| | 1-31, | VL | 148 | DIVMTQAAPSVSVTPGESVSISCTSSKSLLHSNGNTYLYWF |
| | 1-32 | | | LQRPGQSPQLLIYRMSNLASGVPDRFSGSGSGTAFTLRISR |
| | | | | VEAEDVGVYYCMQHREYPFTFGSGTKLEIK |
| (B-26) | 1-33, | VH | 149 | EVQLQQSGPELVKPGASVKISCKTSGYTFTEYTIHWVKQSH |
| | 1-34, | | | GESLEWVGGINPNNGNTRYDQKFKGKATLTIDKSSSPAYM |
| | 1-35, | | | ELRSLTSEDSAVYYCARVARSSGSGPYAMDYWGQGTSVT |
| | 1-36, | | | VSS |
| | 1-37, | VL | 150 | DVVMTQTPLSLPVSLGDQASISCRCTQSLLHTNGDTYLHW |
| | 1-38 | | | YLQKPGQSPKLLIYKVSNRFSGVPDRFSGSGSGTDFTLKIS |
| | | | | RVEAEDLGVYFCSQSTHVPYTFGGGTKLEIK |
| (B-27) | 1-39, | VH | 151 | EVQLVESGGGLVQPKGSLKLSCAASGFTFNTYAMNWVRQ |
| | 1-40, | | | APGKGLEWVARIRSKSNNYATYYADSVKDRFTISRDDSQS |
| | 1-41, | | | MLYLQMNNLKTEDTAMYYCVRGREAYYRYDGGYYAMDY |
| | 1-42 | | | WGQGTSVTVSS |
| | 1-43, | VL | 152 | DIVMTQAAPSVPVTPGESVSISCRSSKSLLHSNGNTYLYWF |
| | 1-44 | | | LQRPGQSPQLLIYRMSNLASGVPDRFSGSGSGTAFTLRISR |
| | | | | VEAEDVGVYYCMQHLEYPFTFGSGTKLEIK |
| (B-28) | 1-45, | VH | 153 | QVQLVETGGGLVRPGNSLKLSCVTSGFTFSNYRMHWLRQ |
| | 1-46, | | | PPGKRLEWIAVIKVKSDNYGANYAESVKGRFTISRDDSKSS |
| | 1-47, | | | VYLQVNRLREEDTATYYCSSPTYPGSSGFAYWGQGTLVTV |
| | 1-48, | | | SA |
| | 1-49, | VL | 154 | DIQMTQTTSSLSASLGDRVTISCRASQDISNYLNWYQQKPD |
| | 1-50 | | | GTVKLLIYYTSRLHSGVPSRFSGSGSGTDYSLTISNLEQEDI |
| | | | | ATYFCQQGNKFPPTFGGGTKLEIN |

In particularly preferred embodiments, the antibody (or antigen-binding fragment) according to the invention comprises a heavy chain variable domain (VH) and a light chain variable domain (VL), both of which are cumulatively defined by: (i) the above-described options (A-1) and (B-1); or (ii) the above-described options (A-2) and (B-2); or (iii) the above-described options (A-3) and (B-3); or (iv) the above-described options (A-4) and (B-4); or (v) the above-described options (A-5) and (B-5); or (vi) the above-described options (A-6) and (B-6); or (vii) the above-described options (A-7) and (B-7); or (viii) the above-described options (A-8) and (B-8); or (ix) the above-described options (A-9) and (B-9); or (x) the above-described options (A-10) and (B-10); or (xi) the above-described options (A-11) and (B-11); or (xii) the above-described options (A-12) and (B-12); or (xiii) the above-described options (A-13) and (B-13); or (xiv) the above-described options (A-14) and (B-14); or (xv) the above-described options (A-15) and (B-15); or (xvi) the above-described options (A-16) and (B-16); or (xvii) the above-described options (A-17) and (B-17); or (xviii) the above-described options (A-18) and (B-18); or (xix) the above-described options (A-19) and (B-19); or (xx) the above-described options (A-20) and (B-20); or (xxi) the above-described options (A-21) and (B-21); or (xxii) the above-described options (A-22) and (B-22); or (xxiii) the above-described options (A-22) and (B-23); or (xxiv) the above-described options (A-25) and (B-24); or (xxv) the above-described options (A-23) and (B-25); or (xxvi) the above-described options (A-22) and (B-26); or (xxvii) the above-described options (A-24) and (B-27); or (xxviii) the above-described options (A-25) and (B-28).

In preferred embodiments, the antibody (or antigen-binding fragment) according to the invention comprises a heavy chain (HC) and/or a light chain (LC), wherein said HC and said LC each have an amino acid sequence with at least 90% (more preferably at least 93%, even more preferably at least 95%, even more preferably at least 97%, even more preferably at least 98%, yet even more preferably at least 99%, still more preferably 100%) sequence identity to the HC and the LC, respectively, of any one of the exemplary antibodies described in the examples section herein below. In particular, it is preferred that the antibody (or antigen-binding fragment) according to the invention comprises:
(C-1) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVOLVESGGGLVQPGRSLRLSCTASGFTFNTYAMNWVRQAPGKGLEWVARIRSKSNNYATYYAASVKGR FTISRDDSKSIAYLQMNSLKTEDTAVYYCVRGREAYYRYDGGYYAMDVWGQGTTVTVSSASTKGPSVFPL APSSKSTSGGTAALGCLVKDYFPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPKDTLMISRTPEVTCVVVDVSHE DPEVKFNWYVDGVEVHNAKTKPREEQYNSTYRVVSVLTVLHQDWLNGKEYKCKVSNKALPAPIEKTISKA KGQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 159); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSLPVTPGESASISCRSSKSLLHSNGNTYLYWFLQKPGQSPQLLIYRMSNLASGVPDRFSGSG SGTAFTLKISRVEAEDVGVYYCMQHLEYPFTFGSGTKLEIKRTVAAPSVFIFPPSDEQLKSGTASVVCLLNN FYPREAKVQWKVDNALQSGNSQESVTEQDSKDSTYSLSSTLTLSKADYEKHKVYACEVTHQGLSSPVTK SFNRGEC (SEQ ID NO: 160); or
(C-2) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVQLLESGGGLVQPGGSLRLSCAASGFTFNAYAMNWVRQAPGKGLEWVARIRSKSNDYATYYGDSVKG RFTISRDNSKNTLYLQMNSLRAEDTAVYYCVRGREAYYRYDGGYYAMDVWGQGTTVTVSSASTKGPSVF PLAPSSKSTSGGTAALGCLVKDYFPEPVTVSWNSGALTSGVHTFPAVLQSSGLYSLSSVVTVPSSSLGTQ TYICNVNHKPSNTKVDKKVEPKSCDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPPVLDSDGSFFLY SKLTVDKSRWQQGNVFSCSVMHEALHNHYTQKSLSLSPG (SEQ ID NO: 161); and/or (preferably; and) a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSLSVTPGESASISCTSSKSLLHSNGNTYLYWFLQKPGQSPQLLIYRMSNLASGVPDRFSGSG SGTAFTLKISRVEAEDVGVYYCMQHREYPFTFGSGTKLEIKRTVAAPSVFIFPPSDEQLKSGTASVVCLLNN FYPREAKVQWKVDNALQSGNSQESVTEQDSKDSTYSLSSTLTLSKADYEKHKVYACEVTHQGLSSPVTK SFNRGEC (SEQ ID NO: 162); or
(C-3) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVQLLESGGGLVQPGGSLRLSCAASGFTFNAYAMNWVRQAPGKGLEWVARIRSKSNDYATYYGDSVKG RFTISRDNSKNTLYLQMNSLRAEDTAVYYCVRGREAYYRYDGGYYAMDVWGQGTTVTVSSASTKGPSVF PLAPSSKSTSGGTAALGCLVKDYFPEPVTVSWNSGALTSGVHTFPAVLQSSGLYSLSSVVTVPSSSLGTQ TYICNVNHKPSNTKVDKKVEPKSCDKTHTCPPCPAPELLGGPDVFLFPPKPKDTLMISRTPEVTCVVVDVS HEDPEVKFNWYVDGVEVHNAKTKPREEQYNSTYRVVSVLTVLHQDWLNGKEYKCKVSNKALPAPEEKTI SKAKGQPREPQVYTLPPSRDELTKNQVSLTCLVKGFYPSDIAVEWESNGQPENNYKTTPPVLDSDGSFFL YSKLTVDKSRWQQGNVFSCSVMHEALHNHYTQKSLSLSPG (SEQ ID NO: 163); and/or (preferably; and) a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSLSVTPGESASISCTSSKSLLHSNGNTYLYWFLQKPGQSPQLLIYRMSNLASGVPDRFSGSG SGTAFTLKISRVEAEDVGVYYCMQHREYPFTFGSGTKLEIKRTVAAPSVFIFPPSDEQLKSGTASVVCLLNN FYPREAKVQWKVDNALQSGNSQESVTEQDSKDSTYSLSSTLTLSKADYEKHKVYACEVTHQGLSSPVTK SFNRGEC (SEQ ID NO: 162); or
(C-4) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVQLLESGGGLVQPGGSLRLSCAASGFTFNAYAMNWVRQAPGKGLEWVARIRSKSNDYATYYGDSVKG RFTISRDNSKNTLYLQMNSLRAEDTAVYYCVRGREAYYRYDGGYYAMDVWGQGTTVTVSSASTKGPSVF PLAPSSKSTSGGTAALGCLVKDYFPEPVTVSWNSGALTSGVHTFPAVLQSSGLYSLSSVVTVPSSSLGTQ TYICNVNHKPSNTKVDKKVEPKSCDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPPVLDSDGSFFLY SKLTVDKSRWQQGNVFSCSVMHEALHNHYTQKSLSLSPG (SEQ ID NO: 161); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQSPLSLPVTPGEPASISCRSSKSLLHSNGNTYLYWFLQKPGQSPQLLIYRVSNLASGVPDRFSGSG SGTDFTLKISRVEAEDVGVYYCMQHREYPFTFGQGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 164); or
(C-5) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence QVQLVQSGPEVVKPGASVKVSCKTSGYTFTEYTIHWVRQAPGQSLEWVGGINPNNGNTRYDQKFQGRV TITRDKSASTAYMELRSLTSEDTAVYYCARVARSSGSGPYAMDYWGQGTTVTVSSASTKGPSVFPLAPSS KSTSGGTAALGCLVKDYFPEPVTVSWNSGALTSGVHTFPAVLQSSGLYSLSSVVTVPSSSLGTQTYICNVN HKPSNTKVDKKVEPKSCDKTHTCPPCPAPELLGGPSVFLFPPKPKDTLMISRTPEVTCVVVDVSHEDPEVK FNWYVDGVEVHNAKTKPREEQYNSTYRVVSVLTVLHQDWLNGKEYKCKVSNKALPAPIEKTISKAKGQPR EPQVYTLPPSRDELTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 165); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DVVMTQSPLSLPVTLGDQASISCRCSQSLLHTNGDTYLHWYLQRPGQSPRLLIYKVSNRFSGVPDRFSGS GSGTDFTLKISRVEAEDVGVYFCSQSTHVPYTFGGGTKVEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 166); or
(C-6) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence QVQLQQSGPEVVKPGASVKVSCKTSGYTFTEYTIHWVRQSHGQSLEWVGGINPNNGNTRYDQKFKGRV TITIDKSSSTAYMELRSLTSEDTAVYYCARVARSSGSGPYAMDYWGQGTTVTVSSASTKGPSVFPLAPSS KSTSGGTAALGCLVKDYFPEPVTVSWNSGALTSGVHTFPAVLQSSGLYSLSSVVTVPSSSLGTQTYICNVN HKPSNTKVDKKVEPKSCDKTHTCPPCPAPELLGGPSVFLFPPKPKDTLMISRTPEVTCVVVDVSHEDPEVK FNWYVDGVEVHNAKTKPREEQYNSTYRVVSVLTVLHQDWLNGKEYKCKVSNKALPAPIEKTISKAKGQPR EPQVYTLPPSRDELTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 167); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DVVMTQSPLSLPVTLGDQASISCRCSQSLLHTNGDTYLHWYLQRPGQSPRLLIYKVSNRFSGVPDRFSGS GSGTDFTLKISRVEAEDVGVYFCSQSTHVPYTFGGGTKVEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 166); or
(C-7) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence QVQLVESGGGVVQPGRSLRLSCAASGFTFSNYRMHWVRQAPGKGLEWIAVIKVKSDNYGANYADSVKG RFTISRDNSKNTLYLQMNSLRAEDTAVYYCSSPTYPGSSGFAYWGQGTLVTVSSASTKGPSVFPLAPSSK STSGGTAALGCLVKDYFPEPVTVSWNSGALTSGVHTFPAVLQSSGLYSLSSVVTVPSSSLGTQTYICNVN HKPSNTKVDKKVEPKSCDKTHTCPPCPAPELLGGPSVFLFPPKPKDTLMISRTPEVTCVVVDVSHEDPEVK FNWYVDGVEVHNAKTKPREEQYNSTYRVVSVLTVLHQDWLNGKEYKCKVSNKALPAPIEKTISKAKGQPR EPQVYTLPPSRDELTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 168); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIQMTQSPSSLSASVGDRVTITCRASQDISNYLNWYQQKPDGTVKLLIYYTSRLHTGVPSRFSGSGSGTDY TLTISNLQQEDIATYFCQQGNKFPPTFGGGTKVEINRTVAAPSVFIFPPSDEQLKSGTASVVCLLNNFYPRE AKVQWKVDNALQSGNSQESVTEQDSKDSTYSLSSTLTLSKADYEKHKVYACEVTHQGLSSPVTKSFNRG EC (SEQ ID NO: 169); or
(C-8) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVOLVESGGGLVQPTGSLRLSCAASGFTFNAYAMNWVRQAPGKGLEWVARIRSKSNDYATYYGDSVKD RFTISRDDSQSMLYLQMNNLKTEDTAMYYCVRGREAYYRYDGGYYAMDYWGQGTSVTVSSASTKGPSV FPLAPSSKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFPPKPKDTLMISRTPEVTCVVVDV SHEDPEVKFNWYVDGVEVHNAKTKPREEQYNSTYRVVSVLTVLHQDWLNGKEYKCKVSNKALPAPIEKTI SKAKGQPREPQVYTLPPSRDELTKNQVSLTCLVKGFYPSDIAVEWESNGQPENNYKTTPPVLDSDGSFFL YSKLTVDKSRWQQGNVFSCSVMHEALHNHYTQKSLSLSPG (SEQ ID NO: 170); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSVSVTPGESVSISCTSSKSLLHSNGNTYLYWFLQRPGQSPQLLIYRMSNLASGVPDRFSGS GSGTAFTLRISRVEAEDVGVYYCMQHREYPFTFGSGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 171); or
(C-9) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVOLVESGGGLVQPTGSLRLSCAASGFTFNAYAMNWVRQAPGKGLEWVARIRSKSNDYATYYGDSVKD RFTISRDDSQSMLYLQMNNLKTEDTAMYYCVRGREAYYRYDGGYYAMDYWGQGTSVTVSSASTKGPSV FPLAPSSKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPDVFLFPPKPKDTLMISRTPEVTCVVVDV SHEDPEVKFNWYVDGVEVHNAKTKPREEQYNSTYRVVSVLTVLHQDWLNGKEYKCKVSNKALPAPEEKT ISKAKGQPREPQVYTLPPSRDELTKNQVSLTCLVKGFYPSDIAVEWESNGQPENNYKTTPPVLDSDGSFFL YSKLTVDKSRWQQGNVFSCSVMHEALHNHYTQKSLSLSPG (SEQ ID NO: 172); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSVSVTPGESVSISCTSSKSLLHSNGNTYLYWFLQRPGQSPQLLIYRMSNLASGVPDRFSGS GSGTAFTLRISRVEAEDVGVYYCMQHREYPFTFGSGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 171); or
(C-10) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVOLVESGGGLVQPTGSLRLSCAASGFTFNAYAMNWVRQAPGKGLEWVARIRSKSNDYATYYGDSVKD RFTISRDDSQSMLYLQMNNLKTEDTAMYYCVRGREAYYRYDGGYYAMDYWGQGTSVTVSSASTKGPSV FPLAPSSKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPDVFLFPPKPKDTLMISRTPEVTCVVVDV SHEDPEVKFNWYVDGVEVHNAKTKPREEQYNSTYRVVSVLTVLHQDWLNGKEYKCKVSNKALPLPEEKTI SKAKGQPREPQVYTLPPSRDELTKNQVSLTCLVKGFYPSDIAVEWESNGQPENNYKTTPPVLDSDGSFFL YSKLTVDKSRWQQGNVFSCSVMHEALHNHYTQKSLSLSPG (SEQ ID NO: 173); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSVSVTPGESVSISCTSSKSLLHSNGNTYLYWFLQRPGQSPQLLIYRMSNLASGVPDRFSGS GSGTAFTLRISRVEAEDVGVYYCMQHREYPFTFGSGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 171); or
(C-11) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVOLVESGGGLVQPTGSLRLSCAASGFTFNAYAMNWVRQAPGKGLEWVARIRSKSNDYATYYGDSVKD RFTISRDDSQSMLYLQMNNLKTEDTAMYYCVRGREAYYRYDGGYYAMDYWGQGTSVTVSSASTKGPSV FPLAPSSKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELVGGPSVFLLPPKPKDTLMISRTPEVTCVVVDV SHEDPEVKFNWYVDGVEVHNAKTKPPEEQYNSTLRVVSVLTVLHQDWLNGKEYKCKVSNKALPAPIEKTI SKAKGQPREPQVYTLPPSRDELTKNQVSLTCLVKGFYPSDIAVEWESNGQPENNYKTTPLVLDSDGSFFL YSKLTVDKSRWQQGNVFSCSVMHEALHNHYTQKSLSLSPG (SEQ ID NO: 174); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSVSVTPGESVSISCTSSKSLLHSNGNTYLYWFLQRPGQSPQLLIYRMSNLASGVPDRFSGS GSGTAFTLRISRVEAEDVGVYYCMQHREYPFTFGSGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 171); or
(C-12) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVOLVESGGGLVQPTGSLRLSCAASGFTFNAYAMNWVRQAPGKGLEWVARIRSKSNDYATYYGDSVKD RFTISRDDSQSMLYLQMNNLKTEDTAMYYCVRGREAYYRYDGGYYAMDYWGQGTSVTVSSASTKGPSV FPLAPSSKSTSGGTAALGCLVKDYFPEPVTVSWNSGALTSGVHTFPAVLQSSGLYSLSSVTVPSSSLGT QTYICNVNHKPSNTKVDKKVEPKSCDKTHTCPPCPAPELVGGPSVFLLPPKPKDTLMISRTPEVTCVVVDV SHEDPEVKFNWYVDGVEVHNAKTKPPEEQYNSTLRVVSVLTVLHQDWLNGKEYKCKVSNWALPAPISKTI SKAKGQPREPQVYTLPPSRDELTKNQVSLTCLVKGFYPSDIAVEWESNGQPENNYKTTPLVLDSDGSFFL YSKLTVDKSRWQQGNVFSCSVMHEALHNHYTQKSLSLSPG (SEQ ID NO: 175); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSVSVTPGESVSISCTSSKSLLHSNGNTYLYWFLQRPGQSPQLLIYRMSNLASGVPDRFSGS GSGTAFTLRISRVEAEDVGVYYCMQHREYPFTFGSGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 171); or
(C-13) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVQLQQSGPELVKPGASVKISCKTSGYTFTEYTIHWVKQSHGESLEWVGGINPNNGNTRYDQKFKGKATL TIDKSSSPAYMELRSLTSEDSAVYYCARVARSSGSGPYAMDYWGQGTSVTVSSASTKGPSVFPLAPSSKS TSGGTAALGCLVKDYFPEPVTVSWNSGALTSGVHTFPAVLQSSGLYSLSSVVTVPSSSLGTQTYICNVNH KPSNTKVDKKVEPKSCDKTHTCPPCPAPELLGGPSVFLFPPKPKDTLMISRTPEVTCVVVDVSHEDPEVKF NWYVDGVEVHNAKTKPREEQYNSTYRVVSVLTVLHQDWLNGKEYKCKVSNKALPAPIEKTISKAKGQPRE PQVYTLPPSRDELTKNQVSLTCLVKGFYPSDIAVEWESNGQPENNYKTTPPVLDSDGSFFLYSKLTVDKSR WQQGNVFSCSVMHEALHNHYTQKSLSLSPG (SEQ ID NO: 176); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DVVMTQTPLSLPVSLGDQASISCRCTQSLLHTNGDTYLHWYLQKPGQSPKLLIYKVSNRFSGVPDRFSGS GSGTDFTLKISRVEAEDLGVYFCSQSTHVPYTFGGGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 177); or
(C-14) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVQLQQSGPELVKPGASVKISCKTSGYTFTEYTIHWVKQSHGESLEWVGGINPNNGNTRYDQKFKGKATL TIDKSSSPAYMELRSLTSEDSAVYYCARVARSSGSGPYAMDYWGQGTSVTVSSASTKGPSVFPLAPSSKS TSGGTAALGCLVKDYFPEPVTVSWNSGALTSGVHTFPAVLQSSGLYSLSSVVTVPSSSLGTQTYICNVNH KPSNTKVDKKVEPKSCDKTHTCPPCPAPELLGGPDVFLFPPKPKDTLMISRTPEVTCVVVDVSHEDPEVKF NWYVDGVEVHNAKTKPREEQYNSTYRVVSVLTVLHQDWLNGKEYKCKVSNKALPAPEEKTISKAKGQPR EPQVYTLPPSRDELTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 178); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DWVMTQTPLSLPVSLGDQASISCRCTQSLLHTNGDTYLHWYLQKPGQSPKLLIYKVSNRFSGVPDRFSGS GSGTDFTLKISRVEAEDLGVYFCSQSTHVPYTFGGGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 177); or
(C-15) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVQLQQSGPELVKPGASVKISCKTSGYTFTEYTIHWVKQSHGESLEWVGGINPNNGNTRYDQKFKGKATL TIDKSSSPAYMELRSLTSEDSAVYYCARVARSSGSGPYAMDYWGQGTSVTVSSASTKGPSVFPLAPSSKS TSGGTAALGCLVKDYFPEPVTVSWNSGALTSGVHTFPAVLQSSGLYSLSSVVTVPSSSLGTQTYICNVNH KPSNTKVDKKVEPKSCDKTHTCPPCPAPELLGGPDVFLFPPKPKDTLMISRTPEVTCVVVDVSHEDPEVKF NWYVDGVEVHNAKTKPREEQYNSTYRVVSVLTVLHQDWLNGKEYKCKVSNKALPLPEEKTISKAKGQPR EPQVYTLPPSRDELTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 179); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DWVMTQTPLSLPVSLGDQASISCRCTQSLLHTNGDTYLHWYLQKPGQSPKLLIYKVSNRFSGVPDRFSGS GSGTDFTLKISRVEAEDLGVYFCSQSTHVPYTFGGGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 177); or
(C-16) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVQLQQSGPELVKPGASVKISCKTSGYTFTEYTIHWVKQSHGESLEWVGGINPNNGNTRYDQKFKGKATL TIDKSSSPAYMELRSLTSEDSAVYYCARVARSSGSGPYAMDYWGQGTSVTVSSASTKGPSVFPLAPSSKS TSGGTAALGCLVKDYFPEPVTVSWNSGALTSGVHTFPAVLQSSGLYSLSSVVTVPSSSLGTQTYICNVNH KPSNTKVDKKVEPKSCDKTHTCPPCPAPELVGGPSVFLLPPKPKDTLMISRTPEVTCVVVDVSHEDPEVKF NWYVDGVEVHNAKTKPPEEQYNSTLRVVSVLTVLHQDWLNGKEYKCKVSNKALPAPIEKTISKAKGQPRE PQVYTLPPSRDELTKNQVSLTCLVKGFYPSDIAVEWESNGQPENNYKTTPLVLDSDGSFFLYSKLTVDKSR WQQGNVFSCSVMHEALHNHYTQKSLSLSPG (SEQ ID NO: 180); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DWVMTQTPLSLPVSLGDQASISCRCTQSLLHTNGDTYLHWYLQKPGQSPKLLIYKVSNRFSGVPDRFSGS GSGTDFTLKISRVEAEDLGVYFCSQSTHVPYTFGGGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 177); or
(C-17) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVQLQQSGPELVKPGASVKISCKTSGYTFTEYTIHWVKQSHGESLEWVGGINPNNGNTRYDQKFKGKATL TIDKSSSPAYMELRSLTSEDSAVYYCARVARSSGSGPYAMDYWGQGTSVTVSSASTKGPSVFPLAPSSKS TSGGTAALGCLVKDYFPEPVTVSWNSGALTSGVHTFPAVLQSSGLYSLSSVVTVPSSSLGTQTYICNVNH KPSNTKVDKKVEPKSCDKTHTCPPCPAPELVGGPSVFLLPPKPKDTLMISRTPEVTCVVVDVSHEDPEVKF NWYVDGVEVHNAKTKPPEEQYNSTLRVVSVLTVLHQDWLNGKEYKCKVSNWALPAPISKTISKAKGQPRE PQVYTLPPSRDELTKNQVSLTCLVKGFYPSDIAVEWESNGQPENNYKTTPLVLDSDGSFFLYSKLTVDKSR WQQGNVFSCSVMHEALHNHYTQKSLSLSPG (SEQ ID NO: 181); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DVVMTQTPLSLPVSLGDQASISCRCTQSLLHTNGDTYLHWYLQKPGQSPKLLIYKVSNRFSGVPDRFSGS GSGTDFTLKISRVEAEDLGVYFCSQSTHVPYTFGGGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 177); or
(C-18) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVOLVESGGGLVQPKGSLKLSCAASGFTFNTYAMNWVRQAPGKGLEWVARIRSKSNNYATYYADSVKDR FTISRDDSQSMLYLQMNNLKTEDTAMYYCVRGREAYYRYDGGYYAMDYWGQGTSVTVSSASTKGPSVF PLAPSSKSTSGGTAALGCLVKDYFPEPVTVSWNSGALTSGVHTFPAVLQSSGLYSLSSVVTVPSSSLGTQ TYICNVNHKPSNTKVDKKVEPKSCDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPPVLDSDGSFFLY SKLTVDKSRWQQGNVFSCSVMHEALHNHYTQKSLSLSPG (SEQ ID NO: 182); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSVPVTPGESVSISCRSSKSLLHSNGNTYLYWFLQRPGQSPQLLIYRMSNLASGVPDRFSGS GSGTAFTLRISRVEAEDVGVYYCMQHLEYPFTFGSGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 183); or
(C-19) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVOLVESGGGLVQPKGSLKLSCAASGFTFNTYAMNWVRQAPGKGLEWVARIRSKSNNYATYYADSVKDR FTISRDDSQSMLYLQMNNLKTEDTAMYYCVRGREAYYRYDGGYYAMDYWGQGTSVTVSSASTKGPSVF PLAPSSKSTSGGTAALGCLVKDYFPEPVTVSWNSGALTSGVHTFPAVLQSSGLYSLSSVVTVPSSSLGTQ TYICNVNHKPSNTKVDKKVEPKSCDKTHTCPPCPAPELLGGPDVFLFPPKPKDTLMISRTPEVTCVVVDVS HEDPEVKFNWYVDGVEVHNAKTKPREEQYNSTYRVVSVLTVLHQDWLNGKEYKCKVSNKALPAPEEKTI SKAKGQPREPQVYTLPPSRDELTKNQVSLTCLVKGFYPSDIAVEWESNGQPENNYKTTPPVLDSDGSFFL YSKLTVDKSRWQQGNVFSCSVMHEALHNHYTQKSLSLSPG (SEQ ID NO: 184); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSVPVTPGESVSISCRSSKSLLHSNGNTYLYWFLQRPGQSPQLLIYRMSNLASGVPDRFSGS GSGTAFTLRISRVEAEDVGVYYCMQHLEYPFTFGSGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 183); or
(C-20) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVOLVESGGGLVQPKGSLKLSCAASGFTFNTYAMNWVRQAPGKGLEWVARIRSKSNNYATYYADSVKDR FTISRDDSQSMLYLQMNNLKTEDTAMYYCVRGREAYYRYDGGYYAMDYWGQGTSVTVSSASTKGPSVF PLAPSSKSTSGGTAALGCLVKDYFPEPVTVSWNSGALTSGVHTFPAVLQSSGLYSLSSVVTVPSSSLGTQ TYICNVNHKPSNTKVDKKVEPKSCDKTHTCPPCPAPELLGGPDVFLFPPKPKDTLMISRTPEVTCVVVDVS HEDPEVKFNWYVDGVEVHNAKTKPREEQYNSTYRVVSVLTVLHQDWLNGKEYKCKVSNKALPLPEEKTIS KAKGQPREPQVYTLPPSRDELTKNQVSLTCLVKGFYPSDIAVEWESNGQPENNYKTTPPVLDSDGSFFLY SKLTVDKSRWQQGNVFSCSVMHEALHNHYTQKSLSLSPG (SEQ ID NO: 185); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSVPVTPGESVSISCRSSKSLLHSNGNTYLYWFLQRPGQSPQLLIYRMSNLASGVPDRFSGS GSGTAFTLRISRVEAEDVGVYYCMQHLEYPFTFGSGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 183); or
(C-21) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVQLVESGGGLVQPKGSLKLSCAASGFTFNTYAMNWVRQAPGKGLEWVARIRSKSNNYATYYADSVKDR FTISRDDSQSMLYLQMNNLKTEDTAMYYCVRGREAYYRYDGGYYAMDYWGQGTSVTVSSASTKGPSVF PLAPSSKSTSGGTAALGCLVKDYFPEPVTVSWNSGALTSGVHTFPAVLQSSGLYSLSSVVTVPSSSLGTQ TYICNVNHKPSNTKVDKKVEPKSCDKTHTCPPCPAPELVGGPSVFLLPPKPKDTLMISRTPEVTCVVVDVS HEDPEVKFNWYVDGVEVHNAKTKPPEEQYNSTLRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPLVLDSDGSFFLY SKLTVDKSRWQQGNVFSCSVMHEALHNHYTQKSLSLSPG (SEQ ID NO: 186); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSVPVTPGESVSISCRSSKSLLHSNGNTYLYWFLQRPGQSPQLLIYRMSNLASGVPDRFSGS GSGTAFTLRISRVEAEDVGVYYCMQHLEYPFTFGSGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 183); or
(C-22) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence EVOLVESGGGLVQPKGSLKLSCAASGFTFNTYAMNWVRQAPGKGLEWVARIRSKSNNYATYYADSVKDR FTISRDDSQSMLYLQMNNLKTEDTAMYYCVRGREAYYRYDGGYYAMDYWGQGTSVTVSSASTKGPSVF PLAPSSKSTSGGTAALGCLVKDYFPEPVTVSWNSGALTSGVHTFPAVLQSSGLYSLSSVVTVPSSSLGTQ TYICNVNHKPSNTKVDKKVEPKSCDKTHTCPPCPAPELVGGPSVFLLPPKPKDTLMISRTPEVTCVVVDVS HEDPEVKFNWYVDGVEVHNAKTKPPEEQYNSTLRVVSVLTVLHQDWLNGKEYKCKVSNWALPAPISKTIS KAKGQPREPQVYTLPPSRDELTKNQVSLTCLVKGFYPSDIAVEWESNGQPENNYKTTPLVLDSDGSFFLY SKLTVDKSRWQQGNVFSCSVMHEALHNHYTQKSLSLSPG (SEQ ID NO: 187); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIVMTQAAPSVPVTPGESVSISCRSSKSLLHSNGNTYLYWFLQRPGQSPQLLIYRMSNLASGVPDRFSGS GSGTAFTLRISRVEAEDVGVYYCMQHLEYPFTFGSGTKLEIKRTVAAPSVFIFPPSDEQLKSGTASVVCLLN NFYPREAKVQWKVDNALQSGNSQESVTEQDSKDSTYSLSSTLTLSKADYEKHKVYACEVTHQGLSSPVT KSFNRGEC (SEQ ID NO: 183); or
(C-23) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence QVQLVETGGGLVRPGNSLKLSCVTSGFTFSNYRMHWLRQPPGKRLEWIAVIKVKSDNYGANYAESVKGR FTISRDDSKSSVYLQVNRLREEDTATYYCSSPTYPGSSGFAYWGQGTLVTVSAASTKGPSVFPLAPSSKS TSGGTAALGCLVKDYFPEPVTVSWNSGALTSGVHTFPAVLQSSGLYSLSSVVTVPSSSLGTQTYICNVNH KPSNTKVDKKVEPKSCDKTHTCPPCPAPELLGGPSVFLFPPKPKDTLMISRTPEVTCVVVDVSHEDPEVKF NWYVDGVEVHNAKTKPREEQYNSTYRVVSVLTVLHQDWLNGKEYKCKVSNKALPAPIEKTISKAKGQPRE PQVYTLPPSRDELTKNQVSLTCLVKGFYPSDIAVEWESNGQPENNYKTTPPVLDSDGSFFLYSKLTVDKSR WQQGNVFSCSVMHEALHNHYTQKSLSLSPG (SEQ ID NO: 188); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIQMTQTTSSLSASLGDRVTISCRASQDISNYLNWYQQKPDGTVKLLIYYTSRLHSGVPSRFSGSGSGTDY SLTISNLEQEDIATYFCQQGNKFPPTFGGGTKLEINRTVAAPSVFIFPPSDEQLKSGTASVVCLLNNFYPRE AKVQWKVDNALQSGNSQESVTEQDSKDSTYSLSSTLTLSKADYEKHKVYACEVTHQGLSSPVTKSFNRG EC (SEQ ID NO: 189); or
(C-24) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence QVQLVETGGGLVRPGNSLKLSCVTSGFTFSNYRMHWLRQPPGKRLEWIAVIKVKSDNYGANYAESVKGR FTISRDDSKSSVYLQVNRLREEDTATYYCSSPTYPGSSGFAYWGQGTLVTVSAASTKGPSVFPLAPSSKS TSGGTAALGCLVKDYFPEPVTVSWNSGALTSGVHTFPAVLQSSGLYSLSSVVTVPSSSLGTQTYICNVNH KPSNTKVDKKVEPKSCDKTHTCPPCPAPELLGGPDVFLFPPKPKDTLMISRTPEVTCVVVDVSHEDPEVKF NWYVDGVEVHNAKTKPREEQYNSTYRVVSVLTVLHQDWLNGKEYKCKVSNKALPAPEEKTISKAKGQPR EPQVYTLPPSRDELTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 190); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIQMTQTTSSLSASLGDRVTISCRASQDISNYLNWYQQKPDGTVKLLIYYTSRLHSGVPSRFSGSGSGTDY SLTISNLEQEDIATYFCQQGNKFPPTFGGGTKLEINRTVAAPSVFIFPPSDEQLKSGTASVVCLLNNFYPRE AKVQWKVDNALQSGNSQESVTEQDSKDSTYSLSSTLTLSKADYEKHKVYACEVTHQGLSSPVTKSFNRG EC (SEQ ID NO: 189); or
(C-25) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence QVQLVETGGGLVRPGNSLKLSCVTSGFTFSNYRMHWLRQPPGKRLEWIAVIKVKSDNYGANYAESVKGR FTISRDDSKSSVYLQVNRLREEDTATYYCSSPTYPGSSGFAYWGQGTLVTVSAASTKGPSVFPLAPSSKS TSGGTAALGCLVKDYFPEPVTVSWNSGALTSGVHTFPAVLQSSGLYSLSSWVTVPSSSLGTQTYICNVNH KPSNTKVDKKVEPKSCDKTHTCPPCPAPELLGGPDVFLFPPKPKDTLMISRTPEVTCVVVDVSHEDPEVKF NWYVDGVEVHNAKTKPREEQYNSTYRVVSVLTVLHQDWLNGKEYKCKVSNKALPLPEEKTISKAKGQPR EPQVYTLPPSRDELTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 191); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIQMTQTTSSLSASLGDRVTISCRASQDISNYLNWYQQKPDGTVKLLIYYTSRLHSGVPSRFSGSGSGTDY SLTISNLEQEDIATYFCQQGNKFPPTFGGGTKLEINRTVAAPSVFIFPPSDEQLKSGTASVVCLLNNFYPRE AKVQWKVDNALQSGNSQESVTEQDSKDSTYSLSSTLTLSKADYEKHKVYACEVTHQGLSSPVTKSFNRG EC (SEQ ID NO: 189); or
(C-26) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence QVQLVETGGGLVRPGNSLKLSCVTSGFTFSNYRMHWLRQPPGKRLEWIAVIKVKSDNYGANYAESVKGR FTISRDDSKSSVYLQVNRLREEDTATYYCSSPTYPGSSGFAYWGQGTLVTVSAASTKGPSVFPLAPSSKS TSGGTAALGCLVKDYFPEPVTVSWNSGALTSGVHTFPAVLQSSGLYSLSSVVTVPSSSLGTQTYICNVNH KPSNTKVDKKVEPKSCDKTHTCPPCPAPELVGGPSVFLLPPKPKDTLMISRTPEVTCVVVDVSHEDPEVKF NWYVDGVEVHNAKTKPPEEQYNSTLRVVSVLTVLHQDWLNGKEYKCKVSNKALPAPIEKTISKAKGQPRE PQVYTLPPSRDELTKNQVSLTCLVKGFYPSDIAVEWESNGQPENNYKTTPLVLDSDGSFFLYSKLTVDKSR WQQGNVFSCSVMHEALHNHYTQKSLSLSPG (SEQ ID NO: 192); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIQMTQTTSSLSASLGDRVTISCRASQDISNYLNWYQQKPDGTVKLLIYYTSRLHSGVPSRFSGSGSGTDY SLTISNLEQEDIATYFCQQGNKFPPTFGGGTKLEINRTVAAPSVFIFPPSDEQLKSGTASVVCLLNNFYPRE AKVQWKVDNALQSGNSQESVTEQDSKDSTYSLSSTLTLSKADYEKHKVYACEVTHQGLSSPVTKSFNRG EC (SEQ ID NO: 189); or
(C-27) a heavy chain (HC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence QVQLVETGGGLVRPGNSLKLSCVTSGFTFSNYRMHWLRQPPGKRLEWIAVIKVKSDNYGANYAESVKGR FTISRDDSKSSVYLQVNRLREEDTATYYCSSPTYPGSSGFAYWGQGTLVTVSAASTKGPSVFPLAPSSKS TSGGTAALGCLVKDYFPEPVTVSWNSGALTSGVHTFPAVLQSSGLYSLSSVVTVPSSSLGTQTYICNVNH KPSNTKVDKKVEPKSCDKTHTCPPCPAPELVGGPSVFLLPPKPKDTLMISRTPEVTCVVVDVSHEDPEVKF NWYVDGVEVHNAKTKPPEEQYNSTLRVVSVLTVLHQDWLNGKEYKCKVSNWALPAPISKTISKAKGQPRE PQVYTLPPSRDELTKNQVSLTCLVKGFYPSDIAVEWESNGQPENNYKTTPLVLDSDGSFFLYSKLTVDKSR WQQGNVFSCSVMHEALHNHYTQKSLSLSPG (SEQ ID NO: 193); and/or (preferably; and)
   a light chain (LC) having an amino acid sequence with at least 90% (more preferably at least 93%, even more preferably at least 95%, even more preferably at least 97%, even more preferably at least 98%, yet even more preferably at least 99%, still more preferably 100%) sequence identity to the amino acid sequence DIQMTQTTSSLSASLGDRVTISCRASQDISNYLNWYQQKPDGTVKLLIYYTSRLHSGVPSRFSGSGSGTDY SLTISNLEQEDIATYFCQQGNKFPPTFGGGTKLEINRTVAAPSVFIFPPSDEQLKSGTASVVCLLNNFYPRE AKVQWKVDNALQSGNSQESVTEQDSKDSTYSLSSTLTLSKADYEKHKVYACEVTHQGLSSPVTKSFNRG EC (SEQ ID NO: 189).

For each of the above-described options (C-1) to (C-27), the heavy chain and the light chain are each defined by a percent sequence identity to a certain reference sequence, and preferred values (lower endpoints) are indicated for the percent sequence identity in each case. While the sequence identity for any chain can, in principle, be selected independently from the sequence identity for the respective other chain, it is generally preferred that the same percent values (lower endpoints) are selected for the sequence identity of the heavy chain and for the sequence identity of the light chain of the same antibody (or antigen-binding fragment). Thus, for example, if in option (C-3) the heavy chain is chosen to have an amino acid sequence with “at least 99%” sequence identity to SEQ ID NO: 163, then it is preferred to choose the same percent sequence identity for the light chain, i.e., to choose the light chain as having an amino acid sequence with “at least 99%” sequence identity to SEQ ID NO: 162. This analogously applies to the percent sequence identities of any other pairs of heavy-chain and light-chain sequences disclosed herein.

The above-mentioned heavy-chain (HC) and light-chain (LC) sequences are also summarized in the following table:

| | | | SEQ ID | |
|---|---|---|---|---|
| | Antibody | Chain | NO: | Amino acid sequence |
| (C-1) | 1-19 | HC | 159 | EVQLVESGGGLVQPGRSLRLSCTASGFTFNTYAMNWVRQ |
| | | | | APGKGLEWVARIRSKSNNYATYYAASVKGRFTISRDDSKSI |
| | | | | AYLQMNSLKTEDTAVYYCVRGREAYYRYDGGYYAMDVWG |
| | | | | QGTTVTVSSASTKGPSVFPLAPSSKSTSGGTAALGCLVKDY |
| | | | | FPEPVTVSWNSGALTSGVHTFPAVLQSSGLYSLSSVVTVPS |
| | | | | SSLGTQTYICNVNHKPSNTKVDKKVEPKSCDKTHTCPPCPA |
| | | | | PELLGGPSVFLFPPKPKDTLMISRTPEVTCVVVDVSHEDPE |
| | | | | VKFNWYVDGVEVHNAKTKPREEQYNSTYRVVSVLTVLHQD |
| | | | | WLNGKEYKCKVSNKALPAPIEKTISKAKGQPREPQVYTLPP |
| | | | | SRDELTKNQVSLTCLVKGFYPSDIAVEWESNGQPENNYKT |
| | | | | TPPVLDSDGSFFLYSKLTVDKSRWQQGNVFSCSVMHEALH |
| | | | | NHYTQKSLSLSPG |
| | | LC | 160 | DIVMTQAAPSLPVTPGESASISCRSSKSLLHSNGNTYLYWF |
| | | | | LQKPGQSPQLLIYRMSNLASGVPDRFSGSGSGTAFTLKISR |
| | | | | VEAEDVGVYYCMQHLEYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-2) | 1-20 | HC | 161 | EVQLLESGGGLVQPGGSLRLSCAASGFTFNAYAMNWVRQ |
| | | | | APGKGLEWVARIRSKSNDYATYYGDSVKGRFTISRDNSKN |
| | | | | TLYLQMNSLRAEDTAVYYCVRGREAYYRYDGGYYAMDVW |
| | | | | GQGTTVTVSSASTKGPSVFPLAPSSKSTSGGTAALGCLVK |
| | | | | DYFPEPVTVSWNSGALTSGVHTFPAVLQSSGLYSLSSVVT |
| | | | | VPSSSLGTQTYICNVNHKPSNTKVDKKVEPKSCDKTHTCPP |
| | | | | CPAPELLGGPSVFLFPPKPKDTLMISRTPEVTCVVVDVSHE |
| | | | | DPEVKFNWYVDGVEVHNAKTKPREEQYNSTYRVVSVLTVL |
| | | | | HQDWLNGKEYKCKVSNKALPAPIEKTISKAKGQPREPQVYT |
| | | | | LPPSRDELTKNQVSLTCLVKGFYPSDIAVEWESNGQPENN |
| | | | | YKTTPPVLDSDGSFFLYSKLTVDKSRWQQGNVFSCSVMHE |
| | | | | ALHNHYTQKSLSLSPG |
| | | LC | 162 | DIVMTQAAPSLSVTPGESASISCTSSKSLLHSNGNTYLYWF |
| | | | | LQKPGQSPQLLIYRMSNLASGVPDRFSGSGSGTAFTLKISR |
| | | | | VEAEDVGVYYCMQHREYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-3) | 1-21, | HC | 163 | EVQLLESGGGLVQPGGSLRLSCAASGFTFNAYAMNWVRQ |
| | 1-26 | | | APGKGLEWVARIRSKSNDYATYYGDSVKGRFTISRDNSKN |
| | | | | TLYLQMNSLRAEDTAVYYCVRGREAYYRYDGGYYAMDVW |
| | | | | GQGTTVTVSSASTKGPSVFPLAPSSKSTSGGTAALGCLVK |
| | | | | DYFPEPVTVSWNSGALTSGVHTFPAVLQSSGLYSLSSVVT |
| | | | | VPSSSLGTQTYICNVNHKPSNTKVDKKVEPKSCDKTHTCPP |
| | | | | CPAPELLGGPDVFLFPPKPKDTLMISRTPEVTCVVVDVSHE |
| | | | | DPEVKFNWYVDGVEVHNAKTKPREEQYNSTYRVVSVLTVL |
| | | | | HQDWLNGKEYKCKVSNKALPAPEEKTISKAKGQPREPQVY |
| | | | | TLPPSRDELTKNQVSLTCLVKGFYPSDIAVEWESNGQPEN |
| | | | | NYKTTPPVLDSDGSFFLYSKLTVDKSRWQQGNVFSCSVMH |
| | | | | EALHNHYTQKSLSLSPG |
| | | LC | 162 | DIVMTQAAPSLSVTPGESASISCTSSKSLLHSNGNTYLYWF |
| | | | | LQKPGQSPQLLIYRMSNLASGVPDRFSGSGSGTAFTLKISR |
| | | | | VEAEDVGVYYCMQHREYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-4) | 1-22 | HC | 161 | EVQLLESGGGLVQPGGSLRLSCAASGFTFNAYAMNWVRQ |
| | | | | APGKGLEWVARIRSKSNDYATYYGDSVKGRFTISRDNSKN |
| | | | | TLYLQMNSLRAEDTAVYYCVRGREAYYRYDGGYYAMDVW |
| | | | | GQGTTVTVSSASTKGPSVFPLAPSSKSTSGGTAALGCLVK |
| | | | | DYFPEPVTVSWNSGALTSGVHTFPAVLQSSGLYSLSSVVT |
| | | | | VPSSSLGTQTYICNVNHKPSNTKVDKKVEPKSCDKTHTCPP |
| | | | | CPAPELLGGPSVFLFPPKPKDTLMISRTPEVTCVVVDVSHE |
| | | | | DPEVKFNWYVDGVEVHNAKTKPREEQYNSTYRVVSVLTVL |
| | | | | HQDWLNGKEYKCKVSNKALPAPIEKTISKAKGQPREPQVYT |
| | | | | LPPSRDELTKNQVSLTCLVKGFYPSDIAVEWESNGQPENN |
| | | | | YKTTPPVLDSDGSFFLYSKLTVDKSRWQQGNVFSCSVMHE |
| | | | | ALHNHYTQKSLSLSPG |
| | | LC | 164 | DIVMTQSPLSLPVTPGEPASISCRSSKSLLHSNGNTYLYWF |
| | | | | LQKPGQSPQLLIYRVSNLASGVPDRFSGSGSGTDFTLKISR |
| | | | | VEAEDVGVYYCMQHREYPFTFGQGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-5) | 1-23 | HC | 165 | QVQLVQSGPEVVKPGASVKVSCKTSGYTFTEYTIHWVRQA |
| | | | | PGQSLEWVGGINPNNGNTRYDQKFQGRVTITRDKSASTAY |
| | | | | MELRSLTSEDTAVYYCARVARSSGSGPYAMDYWGQGTTV |
| | | | | TVSSASTKGPSVFPLAPSSKSTSGGTAALGCLVKDYFPEPV |
| | | | | TVSWNSGALTSGVHTFPAVLQSSGLYSLSSVVTVPSSSLGT |
| | | | | QTYICNVNHKPSNTKVDKKVEPKSCDKTHTCPPCPAPELLG |
| | | | | GPSVFLFPPKPKDTLMISRTPEVTCVVVDVSHEDPEVKFNW |
| | | | | YVDGVEVHNAKTKPREEQYNSTYRWSVLTVLHQDWLNGK |
| | | | | EYKCKVSNKALPAPIEKTISKAKGQPREPQVYTLPPSRDELT |
| | | | | KNQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPG |
| | | LC | 166 | DVVMTQSPLSLPVTLGDQASISCRCSQSLLHTNGDTYLHW |
| | | | | YLQRPGQSPRLLIYKVSNRFSGVPDRFSGSGSGTDFTLKIS |
| | | | | RVEAEDVGVYFCSQSTHVPYTFGGGTKVEIKRTVAAPSVFI |
| | | | | FPPSDEQLKSGTASWCLLNNFYPREAKVQWKVDNALQSG |
| | | | | NSQESVTEQDSKDSTYSLSSTLTLSKADYEKHKVYACEVTH |
| | | | | QGLSSPVTKSFNRGEC |
| (C-6) | 1-24 | HC | 167 | QVQLQQSGPEVVKPGASVKVSCKTSGYTFTEYTIHWVRQS |
| | | | | HGQSLEWVGGINPNNGNTRYDQKFKGRVTITIDKSSSTAY |
| | | | | MELRSLTSEDTAVYYCARVARSSGSGPYAMDYWGQGTTV |
| | | | | TVSSASTKGPSVFPLAPSSKSTSGGTAALGCLVKDYFPEPV |
| | | | | TVSWNSGALTSGVHTFPAVLQSSGLYSLSSVVTVPSSSLGT |
| | | | | QTYICNVNHKPSNTKVDKKVEPKSCDKTHTCPPCPAPELLG |
| | | | | GPSVFLFPPKPKDTLMISRTPEVTCVVVDVSHEDPEVKFNW |
| | | | | YVDGVEVHNAKTKPREEQYNSTYRVVSVLTVLHQDWLNGK |
| | | | | EYKCKVSNKALPAPIEKTISKAKGQPREPQVYTLPPSRDELT |
| | | | | KNQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPG |
| | | LC | 166 | DVVMTQSPLSLPVTLGDQASISCRCSQSLLHTNGDTYLHW |
| | | | | YLQRPGQSPRLLIYKVSNRFSGVPDRFSGSGSGTDFTLKIS |
| | | | | RVEAEDVGVYFCSQSTHVPYTFGGGTKVEIKRTVAAPSVFI |
| | | | | FPPSDEQLKSGTASWVCLLNNFYPREAKVQWKVDNALQSG |
| | | | | NSQESVTEQDSKDSTYSLSSTLTLSKADYEKHKVYACEVTH |
| | | | | QGLSSPVTKSFNRGEC |
| (C-7) | 1-25 | HC | 168 | QVQLVESGGGVVQPGRSLRLSCAASGFTFSNYRMHWVRQ |
| | | | | APGKGLEWIAVIKVKSDNYGANYADSVKGRFTISRDNSKNT |
| | | | | LYLQMNSLRAEDTAVYYCSSPTYPGSSGFAYWGQGTLVTV |
| | | | | SSASTKGPSVFPLAPSSKSTSGGTAALGCLVKDYFPEPVTV |
| | | | | SWNSGALTSGVHTFPAVLQSSGLYSLSSVVTVPSSSLGTQ |
| | | | | TYICNVNHKPSNTKVDKKVEPKSCDKTHTCPPCPAPELLGG |
| | | | | PSVFLFPPKPKDTLMISRTPEVTCVVVDVSHEDPEVKFNWY |
| | | | | VDGVEVHNAKTKPREEQYNSTYRVVSVLTVLHQDWLNGKE |
| | | | | YKCKVSNKALPAPIEKTISKAKGQPREPQVYTLPPSRDELTK |
| | | | | NQVSLTCLVKGFYPSDIAVEWESNGQPENNYKTTPPVLDS |
| | | | | DGSFFLYSKLTVDKSRWQQGNVFSCSVMHEALHNHYTQK |
| | | | | SLSLSPG |
| | | LC | 169 | DIQMTQSPSSLSASVGDRVTITCRASQDISNYLNWYQQKPD |
| | | | | GTVKLLIYYTSRLHTGVPSRFSGSGSGTDYTLTISNLQQEDI |
| | | | | ATYFCQQGNKFPPTFGGGTKVEINRTVAAPSVFIFPPSDEQ |
| | | | | LKSGTASVVCLLNNFYPREAKVQWKVDNALQSGNSQESVT |
| | | | | EQDSKDSTYSLSSTLTLSKADYEKHKVYACEVTHQGLSSPV |
| | | | | TKSFNRGEC |
| (C-8) | 1-27, | HC | 170 | EVQLVESGGGLVQPTGSLRLSCAASGFTFNAYAMNWVRQ |
| | 1-29 | | | APGKGLEWVARIRSKSNDYATYYGDSVKDRFTISRDDSQS |
| | | | | MLYLQMNNLKTEDTAMYYCVRGREAYYRYDGGYYAMDY |
| | | | | WGQGTSVTVSSASTKGPSVFPLAPSSKSTSGGTAALGCLV |
| | | | | KDYFPEPVTVSWNSGALTSGVHTFPAVLQSSGLYSLSSVV |
| | | | | TVPSSSLGTQTYICNVNHKPSNTKVDKKVEPKSCDKTHTCP |
| | | | | PCPAPELLGGPSVFLFPPKPKDTLMISRTPEVTCVVVDVSH |
| | | | | EDPEVKFNWYVDGVEVHNAKTKPREEQYNSTYRVVSVLTV |
| | | | | LHQDWLNGKEYKCKVSNKALPAPIEKTISKAKGQPREPQVY |
| | | | | TLPPSRDELTKNQVSLTCLVKGFYPSDIAVEWESNGQPEN |
| | | | | NYKTTPPVLDSDGSFFLYSKLTVDKSRWQQGNVFSCSVMH |
| | | | | EALHNHYTQKSLSLSPG |
| | | LC | 171 | DIVMTQAAPSVSVTPGESVSISCTSSKSLLHSNGNTYLYWF |
| | | | | LQRPGQSPQLLIYRMSNLASGVPDRFSGSGSGTAFTLRISR |
| | | | | VEAEDVGVYYCMQHREYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-9) | 1-28 | HC | 172 | EVQLVESGGGLVQPTGSLRLSCAASGFTFNAYAMNWVRQ |
| | | | | APGKGLEWVARIRSKSNDYATYYGDSVKDRFTISRDDSQS |
| | | | | MLYLQMNNLKTEDTAMYYCVRGREAYYRYDGGYYAMDY |
| | | | | WGQGTSVTVSSASTKGPSVFPLAPSSKSTSGGTAALGCLV |
| | | | | KDYFPEPVTVSWNSGALTSGVHTFPAVLQSSGLYSLSSVV |
| | | | | TVPSSSLGTQTYICNVNHKPSNTKVDKKVEPKSCDKTHTCP |
| | | | | PCPAPELLGGPDVFLFPPKPKDTLMISRTPEVTCVVVDVSH |
| | | | | EDPEVKFNWYVDGVEVHNAKTKPREEQYNSTYRVVSVLTV |
| | | | | LHQDWLNGKEYKCKVSNKALPAPEEKTISKAKGQPREPQV |
| | | | | YTLPPSRDELTKNQVSLTCLVKGFYPSDIAVEWESNGQPEN |
| | | | | NYKTTPPVLDSDGSFFLYSKLTVDKSRWQQGNVFSCSVMH |
| | | | | EALHNHYTQKSLSLSPG |
| | | LC | 171 | DIVMTQAAPSVSVTPGESVSISCTSSKSLLHSNGNTYLYWF |
| | | | | LQRPGQSPQLLIYRMSNLASGVPDRFSGSGSGTAFTLRISR |
| | | | | VEAEDVGVYYCMQHREYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-10) | 1-30 | HC | 173 | EVQLVESGGGLVQPTGSLRLSCAASGFTFNAYAMNWVRQ |
| | | | | APGKGLEWVARIRSKSNDYATYYGDSVKDRFTISRDDSQS |
| | | | | MLYLQMNNLKTEDTAMYYCVRGREAYYRYDGGYYAMDY |
| | | | | WGQGTSVTVSSASTKGPSVFPLAPSSKSTSGGTAALGCLV |
| | | | | KDYFPEPVTVSWNSGALTSGVHTFPAVLQSSGLYSLSSVV |
| | | | | TVPSSSLGTQTYICNVNHKPSNTKVDKKVEPKSCDKTHTCP |
| | | | | PCPAPELLGGPDVFLFPPKPKDTLMISRTPEVTCVVVDVSH |
| | | | | EDPEVKFNWYVDGVEVHNAKTKPREEQYNSTYRVVSVLTV |
| | | | | LHQDWLNGKEYKCKVSNKALPLPEEKTISKAKGQPREPQV |
| | | | | YTLPPSRDELTKNQVSLTCLVKGFYPSDIAVEWESNGQPEN |
| | | | | NYKTTPPVLDSDGSFFLYSKLTVDKSRWQQGNVFSCSVMH |
| | | | | EALHNHYTQKSLSLSPG |
| | | LC | 171 | DIVMTQAAPSVSVTPGESVSISCTSSKSLLHSNGNTYLYWF |
| | | | | LQRPGQSPQLLIYRMSNLASGVPDRFSGSGSGTAFTLRISR |
| | | | | VEAEDVGVYYCMQHREYPFTFGSGTKLEIKRTVAAPSVFIF |
| | | | | PPSDEQLKSGTASWVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-11) | 1-31 | HC | 174 | EVQLVESGGGLVQPTGSLRLSCAASGFTFNAYAMNWVRQ |
| | | | | APGKGLEWVARIRSKSNDYATYYGDSVKDRFTISRDDSQS |
| | | | | MLYLQMNNLKTEDTAMYYCVRGREAYYRYDGGYYAMDY |
| | | | | WGQGTSVTVSSASTKGPSVFPLAPSSKSTSGGTAALGCLV |
| | | | | KDYFPEPVTVSWNSGALTSGVHTFPAVLQSSGLYSLSSVV |
| | | | | TVPSSSLGTQTYICNVNHKPSNTKVDKKVEPKSCDKTHTCP |
| | | | | PCPAPELVGGPSVFLLPPKPKDTLMISRTPEVTCVVVDVSH |
| | | | | EDPEVKFNWYVDGVEVHNAKTKPPEEQYNSTLRVVSVLTV |
| | | | | LHQDWLNGKEYKCKVSNKALPAPIEKTISKAKGQPREPQVY |
| | | | | TLPPSRDELTKNQVSLTCLVKGFYPSDIAVEWESNGQPEN |
| | | | | NYKTTPLVLDSDGSFFLYSKLTVDKSRWQQGNVFSCSVMH |
| | | | | EALHNHYTQKSLSLSPG |
| | | LC | 171 | DIVMTQAAPSVSVTPGESVSISCTSSKSLLHSNGNTYLYWF |
| | | | | LQRPGQSPQLLIYRMSNLASGVPDRFSGSGSGTAFTLRISR |
| | | | | VEAEDVGVYYCMQHREYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-12) | 1-32 | HC | 175 | EVQLVESGGGLVQPTGSLRLSCAASGFTFNAYAMNWVRQ |
| | | | | APGKGLEWVARIRSKSNDYATYYGDSVKDRFTISRDDSQS |
| | | | | MLYLQMNNLKTEDTAMYYCVRGREAYYRYDGGYYAMDY |
| | | | | WGQGTSVTVSSASTKGPSVFPLAPSSKSTSGGTAALGCLV |
| | | | | KDYFPEPVTVSWNSGALTSGVHTFPAVLQSSGLYSLSSVV |
| | | | | TVPSSSLGTQTYICNVNHKPSNTKVDKKVEPKSCDKTHTCP |
| | | | | PCPAPELVGGPSVFLLPPKPKDTLMISRTPEVTCVVVDVSH |
| | | | | EDPEVKFNWYVDGVEVHNAKTKPPEEQYNSTLRVVSVLTV |
| | | | | LHQDWLNGKEYKCKVSNWALPAPISKTISKAKGQPREPQV |
| | | | | YTLPPSRDELTKNQVSLTCLVKGFYPSDIAVEWESNGQPEN |
| | | | | NYKTTPLVLDSDGSFFLYSKLTVDKSRWQQGNVFSCSVMH |
| | | | | EALHNHYTQKSLSLSPG |
| | | LC | 171 | DIVMTQAAPSVSVTPGESVSISCTSSKSLLHSNGNTYLYWF |
| | | | | LQRPGQSPQLLIYRMSNLASGVPDRFSGSGSGTAFTLRISR |
| | | | | VEAEDVGVYYCMQHREYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-13) | 1-33, | HC | 176 | EVQLQQSGPELVKPGASVKISCKTSGYTFTEYTIHWVKQSH |
| | 1-35 | | | GESLEWVGGINPNNGNTRYDQKFKGKATLTIDKSSSPAYM |
| | | | | ELRSLTSEDSAVYYCARVARSSGSGPYAMDYWGQGTSVT |
| | | | | VSSASTKGPSVFPLAPSSKSTSGGTAALGCLVKDYFPEPVT |
| | | | | VSWNSGALTSGVHTFPAVLQSSGLYSLSSVVTVPSSSLGT |
| | | | | QTYICNVNHKPSNTKVDKKVEPKSCDKTHTCPPCPAPELLG |
| | | | | GPSVFLFPPKPKDTLMISRTPEVTCVVVDVSHEDPEVKFNW |
| | | | | YVDGVEVHNAKTKPREEQYNSTYRWSVLTVLHQDWLNGK |
| | | | | EYKCKVSNKALPAPIEKTISKAKGQPREPQVYTLPPSRDELT |
| | | | | KNQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPG |
| | | LC | 177 | DVVMTQTPLSLPVSLGDQASISCRCTQSLLHTNGDTYLHW |
| | | | | YLQKPGQSPKLLIYKVSNRFSGVPDRFSGSGSGTDFTLKIS |
| | | | | RVEAEDLGVYFCSQSTHVPYTFGGGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-14) | 1-34 | HC | 178 | EVQLQQSGPELVKPGASVKISCKTSGYTFTEYTIHWVKQSH |
| | | | | GESLEWVGGINPNNGNTRYDQKFKGKATLTIDKSSSPAYM |
| | | | | ELRSLTSEDSAVYYCARVARSSGSGPYAMDYWGQGTSVT |
| | | | | VSSASTKGPSVFPLAPSSKSTSGGTAALGCLVKDYFPEPVT |
| | | | | VSWNSGALTSGVHTFPAVLQSSGLYSLSSVVTVPSSSLGT |
| | | | | QTYICNVNHKPSNTKVDKKVEPKSCDKTHTCPPCPAPELLG |
| | | | | GPDVFLFPPKPKDTLMISRTPEVTCVWVDVSHEDPEVKFNW |
| | | | | YVDGVEVHNAKTKPREEQYNSTYRVVSVLTVLHQDWLNGK |
| | | | | EYKCKVSNKALPAPEEKTISKAKGQPREPQVYTLPPSRDEL |
| | | | | TKNQVSLTCLVKGFYPSDIAVEWESNGQPENNYKTTPPVL |
| | | | | DSDGSFFLYSKLTVDKSRWQQGNVFSCSVMHEALHNHYT |
| | | | | QKSLSLSPG |
| | | LC | 177 | DVVMTQTPLSLPVSLGDQASISCRCTQSLLHTNGDTYLHW |
| | | | | YLQKPGQSPKLLIYKVSNRFSGVPDRFSGSGSGTDFTLKIS |
| | | | | RVEAEDLGVYFCSQSTHVPYTFGGGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-15) | 1-36 | HC | 179 | EVQLQQSGPELVKPGASVKISCKTSGYTFTEYTIHWVKQSH |
| | | | | GESLEWVGGINPNNGNTRYDQKFKGKATLTIDKSSSPAYM |
| | | | | ELRSLTSEDSAVYYCARVARSSGSGPYAMDYWGQGTSVT |
| | | | | VSSASTKGPSVFPLAPSSKSTSGGTAALGCLVKDYFPEPVT |
| | | | | VSWNSGALTSGVHTFPAVLQSSGLYSLSSVVTVPSSSLGT |
| | | | | QTYICNVNHKPSNTKVDKKVEPKSCDKTHTCPPCPAPELLG |
| | | | | GPDVFLFPPKPKDTLMISRTPEVTCVVVDVSHEDPEVKFNW |
| | | | | YVDGVEVHNAKTKPREEQYNSTYRWSVLTVLHQDWLNGK |
| | | | | EYKCKVSNKALPLPEEKTISKAKGQPREPQVYTLPPSRDEL |
| | | | | TKNQVSLTCLVKGFYPSDIAVEWESNGQPENNYKTTPPVL |
| | | | | DSDGSFFLYSKLTVDKSRWQQGNVFSCSVMHEALHNHYT |
| | | | | QKSLSLSPG |
| | | LC | 177 | DVVMTQTPLSLPVSLGDQASISCRCTQSLLHTNGDTYLHW |
| | | | | YLQKPGQSPKLLIYKVSNRFSGVPDRFSGSGSGTDFTLKIS |
| | | | | RVEAEDLGVYFCSQSTHVPYTFGGGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-16) | 1-37 | HC | 180 | EVQLQQSGPELVKPGASVKISCKTSGYTFTEYTIHWVKQSH |
| | | | | GESLEWVGGINPNNGNTRYDQKFKGKATLTIDKSSSPAYM |
| | | | | ELRSLTSEDSAVYYCARVARSSGSGPYAMDYWGQGTSVT |
| | | | | VSSASTKGPSVFPLAPSSKSTSGGTAALGCLVKDYFPEPVT |
| | | | | VSWNSGALTSGVHTFPAVLQSSGLYSLSSVVTVPSSSLGT |
| | | | | QTYICNVNHKPSNTKVDKKVEPKSCDKTHTCPPCPAPELV |
| | | | | GGPSVFLLPPKPKDTLMISRTPEVTCVWDVSHEDPEVKFN |
| | | | | WYVDGVEVHNAKTKPPEEQYNSTLRVVSVLTVLHQDWLN |
| | | | | GKEYKCKVSNKALPAPIEKTISKAKGQPREPQVYTLPPSRD |
| | | | | ELTKNQVSLTCLVKGFYPSDIAVEWESNGQPENNYKTTPLV |
| | | | | LDSDGSFFLYSKLTVDKSRWQQGNVFSCSVMHEALHNHYT |
| | | | | QKSLSLSPG |
| | | LC | 177 | DWVMTQTPLSLPVSLGDQASISCRCTQSLLHTNGDTYLHW |
| | | | | YLQKPGQSPKLLIYKVSNRFSGVPDRFSGSGSGTDFTLKIS |
| | | | | RVEAEDLGVYFCSQSTHVPYTFGGGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-17) | 1-38 | HC | 181 | EVQLQQSGPELVKPGASVKISCKTSGYTFTEYTIHWVKQSH |
| | | | | GESLEWVGGINPNNGNTRYDQKFKGKATLTIDKSSSPAYM |
| | | | | ELRSLTSEDSAVYYCARVARSSGSGPYAMDYWGQGTSVT |
| | | | | VSSASTKGPSVFPLAPSSKSTSGGTAALGCLVKDYFPEPVT |
| | | | | VSWNSGALTSGVHTFPAVLQSSGLYSLSSVVTVPSSSLGT |
| | | | | QTYICNVNHKPSNTKVDKKVEPKSCDKTHTCPPCPAPELV |
| | | | | GGPSVFLLPPKPKDTLMISRTPEVTCVVVDVSHEDPEVKFN |
| | | | | WYVDGVEVHNAKTKPPEEQYNSTLRVVSVLTVLHQDWLN |
| | | | | GKEYKCKVSNWALPAPISKTISKAKGQPREPQVYTLPPSRD |
| | | | | ELTKNQVSLTCLVKGFYPSDIAVEWESNGQPENNYKTTPLV |
| | | | | LDSDGSFFLYSKLTVDKSRWQQGNVFSCSVMHEALHNHYT |
| | | | | QKSLSLSPG |
| | | LC | 177 | DVVMTQTPLSLPVSLGDQASISCRCTQSLLHTNGDTYLHW |
| | | | | YLQKPGQSPKLLIYKVSNRFSGVPDRFSGSGSGTDFTLKIS |
| | | | | RVEAEDLGVYFCSQSTHVPYTFGGGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-18) | 1-39, | HC | 182 | EVQLVESGGGLVQPKGSLKLSCAASGFTFNTYAMNWVRQ |
| | 1-41 | | | APGKGLEWVARIRSKSNNYATYYADSVKDRFTISRDDSQS |
| | | | | MLYLQMNNLKTEDTAMYYCVRGREAYYRYDGGYYAMDY |
| | | | | WGQGTSVTVSSASTKGPSVFPLAPSSKSTSGGTAALGCLV |
| | | | | KDYFPEPVTVSWNSGALTSGVHTFPAVLQSSGLYSLSSVV |
| | | | | TVPSSSLGTQTYICNVNHKPSNTKVDKKVEPKSCDKTHTCP |
| | | | | PCPAPELLGGPSVFLFPPKPKDTLMISRTPEVTCVVVDVSH |
| | | | | EDPEVKFNWYVDGVEVHNAKTKPREEQYNSTYRVVSVLTV |
| | | | | LHQDWLNGKEYKCKVSNKALPAPIEKTISKAKGQPREPQVY |
| | | | | TLPPSRDELTKNQVSLTCLVKGFYPSDIAVEWESNGQPEN |
| | | | | NYKTTPPVLDSDGSFFLYSKLTVDKSRWQQGNVFSCSVMH |
| | | | | EALHNHYTQKSLSLSPG |
| | | LC | 183 | DIVMTQAAPSVPVTPGESVSISCRSSKSLLHSNGNTYLYWF |
| | | | | LQRPGQSPQLLIYRMSNLASGVPDRFSGSGSGTAFTLRISR |
| | | | | VEAEDVGVYYCMQHLEYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-19) | 1-40 | HC | 184 | EVQLVESGGGLVQPKGSLKLSCAASGFTFNTYAMNWVRQ |
| | | | | APGKGLEWVARIRSKSNNYATYYADSVKDRFTISRDDSQS |
| | | | | MLYLQMNNLKTEDTAMYYCVRGREAYYRYDGGYYAMDY |
| | | | | WGQGTSVTVSSASTKGPSVFPLAPSSKSTSGGTAALGCLV |
| | | | | KDYFPEPVTVSWNSGALTSGVHTFPAVLQSSGLYSLSSVV |
| | | | | TVPSSSLGTQTYICNVNHKPSNTKVDKKVEPKSCDKTHTCP |
| | | | | PCPAPELLGGPDVFLFPPKPKDTLMISRTPEVTCVVVDVSH |
| | | | | EDPEVKFNWYVDGVEVHNAKTKPREEQYNSTYRVVSVLTV |
| | | | | LHQDWLNGKEYKCKVSNKALPAPEEKTISKAKGQPREPQV |
| | | | | YTLPPSRDELTKNQVSLTCLVKGFYPSDIAVEWESNGQPEN |
| | | | | NYKTTPPVLDSDGSFFLYSKLTVDKSRWQQGNVFSCSVMH |
| | | | | EALHNHYTQKSLSLSPG |
| | | LC | 183 | DIVMTQAAPSVPVTPGESVSISCRSSKSLLHSNGNTYLYWF |
| | | | | LQRPGQSPQLLIYRMSNLASGVPDRFSGSGSGTAFTLRISR |
| | | | | VEAEDVGVYYCMQHLEYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-20) | 1-42 | HC | 185 | EVQLVESGGGLVQPKGSLKLSCAASGFTFNTYAMNWVRQ |
| | | | | APGKGLEWVARIRSKSNNYATYYADSVKDRFTISRDDSQS |
| | | | | MLYLQMNNLKTEDTAMYYCVRGREAYYRYDGGYYAMDY |
| | | | | WGQGTSVTVSSASTKGPSVFPLAPSSKSTSGGTAALGCLV |
| | | | | KDYFPEPVTVSWNSGALTSGVHTFPAVLQSSGLYSLSSVV |
| | | | | TVPSSSLGTQTYICNVNHKPSNTKVDKKVEPKSCDKTHTCP |
| | | | | PCPAPELLGGPDVFLFPPKPKDTLMISRTPEVTCVVVDVSH |
| | | | | EDPEVKFNWYVDGVEVHNAKTKPREEQYNSTYRVVSVLTV |
| | | | | LHQDWLNGKEYKCKVSNKALPLPEEKTISKAKGQPREPQV |
| | | | | YTLPPSRDELTKNQVSLTCLVKGFYPSDIAVEWESNGQPEN |
| | | | | NYKTTPPVLDSDGSFFLYSKLTVDKSRWQQGNVFSCSVMH |
| | | | | EALHNHYTQKSLSLSPG |
| | | LC | 183 | DIVMTQAAPSVPVTPGESVSISCRSSKSLLHSNGNTYLYWF |
| | | | | LQRPGQSPQLLIYRMSNLASGVPDRFSGSGSGTAFTLRISR |
| | | | | VEAEDVGVYYCMQHLEYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-21) | 1-43 | HC | 186 | EVQLVESGGGLVQPKGSLKLSCAASGFTFNTYAMNWVRQ |
| | | | | APGKGLEWVARIRSKSNNYATYYADSVKDRFTISRDDSQS |
| | | | | MLYLQMNNLKTEDTAMYYCVRGREAYYRYDGGYYAMDY |
| | | | | WGQGTSVTVSSASTKGPSVFPLAPSSKSTSGGTAALGCLV |
| | | | | KDYFPEPVTVSWNSGALTSGVHTFPAVLQSSGLYSLSSVV |
| | | | | TVPSSSLGTQTYICNVNHKPSNTKVDKKVEPKSCDKTHTCP |
| | | | | PCPAPELVGGPSVFLLPPKPKDTLMISRTPEVTCVVVDVSH |
| | | | | EDPEVKFNWYVDGVEVHNAKTKPPEEQYNSTLRVVSVLTV |
| | | | | LHQDWLNGKEYKCKVSNKALPAPIEKTISKAKGQPREPQVY |
| | | | | TLPPSRDELTKNQVSLTCLVKGFYPSDIAVEWESNGQPEN |
| | | | | NYKTTPLVLDSDGSFFLYSKLTVDKSRWQQGNVFSCSVMH |
| | | | | EALHNHYTQKSLSLSPG |
| | | LC | 183 | DIVMTQAAPSVPVTPGESVSISCRSSKSLLHSNGNTYLYWF |
| | | | | LQRPGQSPQLLIYRMSNLASGVPDRFSGSGSGTAFTLRISR |
| | | | | VEAEDVGVYYCMQHLEYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-22) | 1-44 | HC | 187 | EVQLVESGGGLVQPKGSLKLSCAASGFTFNTYAMNWVRQ |
| | | | | APGKGLEWVARIRSKSNNYATYYADSVKDRFTISRDDSQS |
| | | | | MLYLQMNNLKTEDTAMYYCVRGREAYYRYDGGYYAMDY |
| | | | | WGQGTSVTVSSASTKGPSVFPLAPSSKSTSGGTAALGCLV |
| | | | | KDYFPEPVTVSWNSGALTSGVHTFPAVLQSSGLYSLSSVV |
| | | | | TVPSSSLGTQTYICNVNHKPSNTKVDKKVEPKSCDKTHTCP |
| | | | | PCPAPELVGGPSVFLLPPKPKDTLMISRTPEVTCVVVDVSH |
| | | | | EDPEVKFNWYVDGVEVHNAKTKPPEEQYNSTLRVVSVLTV |
| | | | | LHQDWLNGKEYKCKVSNWALPAPISKTISKAKGQPREPQV |
| | | | | YTLPPSRDELTKNQVSLTCLVKGFYPSDIAVEWESNGQPEN |
| | | | | NYKTTPLVLDSDGSFFLYSKLTVDKSRWQQGNVFSCSVMH |
| | | | | EALHNHYTQKSLSLSPG |
| | | LC | 183 | DIVMTQAAPSVPVTPGESVSISCRSSKSLLHSNGNTYLYWF |
| | | | | LQRPGQSPQLLIYRMSNLASGVPDRFSGSGSGTAFTLRISR |
| | | | | VEAEDVGVYYCMQHLEYPFTFGSGTKLEIKRTVAAPSVFIF |
| | | | | PPSDEQLKSGTASVVCLLNNFYPREAKVQWKVDNALQSGN |
| | | | | SQESVTEQDSKDSTYSLSSTLTLSKADYEKHKVYACEVTHQ |
| | | | | GLSSPVTKSFNRGEC |
| (C-23) | 1-45, | HC | 188 | QVQLVETGGGLVRPGNSLKLSCVTSGFTFSNYRMHWLRQ |
| | 1-47 | | | PPGKRLEWIAVIKVKSDNYGANYAESVKGRFTISRDDSKSS |
| | | | | VYLQVNRLREEDTATYYCSSPTYPGSSGFAYWGQGTLVTV |
| | | | | SAASTKGPSVFPLAPSSKSTSGGTAALGCLVKDYFPEPVTV |
| | | | | SWNSGALTSGVHTFPAVLQSSGLYSLSSVVTVPSSSLGTQ |
| | | | | TYICNVNHKPSNTKVDKKVEPKSCDKTHTCPPCPAPELLGG |
| | | | | PSVFLFPPKPKDTLMISRTPEVTCVVVDVSHEDPEVKFNWY |
| | | | | VDGVEVHNAKTKPREEQYNSTYRVVSVLTVLHQDWLNGKE |
| | | | | YKCKVSNKALPAPIEKTISKAKGQPREPQVYTLPPSRDELTK |
| | | | | NQVSLTCLVKGFYPSDIAVEWESNGQPENNYKTTPPVLDS |
| | | | | DGSFFLYSKLTVDKSRWQQGNVFSCSVMHEALHNHYTQK |
| | | | | SLSLSPG |
| | | LC | 189 | DIQMTQTTSSLSASLGDRVTISCRASQDISNYLNWYQQKPD |
| | | | | GTVKLLIYYTSRLHSGVPSRFSGSGSGTDYSLTISNLEQEDI |
| | | | | ATYFCQQGNKFPPTFGGGTKLEINRTVAAPSVFIFPPSDEQ |
| | | | | LKSGTASVVCLLNNFYPREAKVQWKVDNALQSGNSQESVT |
| | | | | EQDSKDSTYSLSSTLTLSKADYEKHKVYACEVTHQGLSSPV |
| | | | | TKSFNRGEC |
| (C-24) | 1-46 | HC | 190 | QVQLVETGGGLVRPGNSLKLSCVTSGFTFSNYRMHWLRQ |
| | | | | PPGKRLEWIAVIKVKSDNYGANYAESVKGRFTISRDDSKSS |
| | | | | VYLQVNRLREEDTATYYCSSPTYPGSSGFAYWGQGTLVTV |
| | | | | SAASTKGPSVFPLAPSSKSTSGGTAALGCLVKDYFPEPVTV |
| | | | | SWNSGALTSGVHTFPAVLQSSGLYSLSSVVTVPSSSLGTQ |
| | | | | TYICNVNHKPSNTKVDKKVEPKSCDKTHTCPPCPAPELLGG |
| | | | | PDVFLFPPKPKDTLMISRTPEVTCVVVDVSHEDPEVKFNWY |
| | | | | VDGVEVHNAKTKPREEQYNSTYRVVSVLTVLHQDWLNGKE |
| | | | | YKCKVSNKALPAPEEKTISKAKGQPREPQVYTLPPSRDELT |
| | | | | KNQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPG |
| | | LC | 189 | DIQMTQTTSSLSASLGDRVTISCRASQDISNYLNWYQQKPD |
| | | | | GTVKLLIYYTSRLHSGVPSRFSGSGSGTDYSLTISNLEQEDI |
| | | | | ATYFCQQGNKFPPTFGGGTKLEINRTVAAPSVFIFPPSDEQ |
| | | | | LKSGTASVVCLLNNFYPREAKVQWKVDNALQSGNSQESVT |
| | | | | EQDSKDSTYSLSSTLTLSKADYEKHKVYACEVTHQGLSSPV |
| | | | | TKSFNRGEC |
| (C-25) | 1-48 | HC | 191 | QVQLVETGGGLVRPGNSLKLSCVTSGFTFSNYRMHWLRQ |
| | | | | PPGKRLEWIAVIKVKSDNYGANYAESVKGRFTISRDDSKSS |
| | | | | VYLQVNRLREEDTATYYCSSPTYPGSSGFAYWGQGTLVTV |
| | | | | SAASTKGPSVFPLAPSSKSTSGGTAALGCLVKDYFPEPVTV |
| | | | | SWNSGALTSGVHTFPAVLQSSGLYSLSSVVTVPSSSLGTQ |
| | | | | TYICNVNHKPSNTKVDKKVEPKSCDKTHTCPPCPAPELLGG |
| | | | | PDVFLFPPKPKDTLMISRTPEVTCVVVDVSHEDPEVKFNWY |
| | | | | VDGVEVHNAKTKPREEQYNSTYRVVSVLTVLHQDWLNGKE |
| | | | | YKCKVSNKALPLPEEKTISKAKGQPREPQVYTLPPSRDELT |
| | | | | KNQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPG |
| | | LC | 189 | DIQMTQTTSSLSASLGDRVTISCRASQDISNYLNWYQQKPD |
| | | | | GTVKLLIYYTSRLHSGVPSRFSGSGSGTDYSLTISNLEQEDI |
| | | | | ATYFCQQGNKFPPTFGGGTKLEINRTVAAPSVFIFPPSDEQ |
| | | | | LKSGTASWVCLLNNFYPREAKVQWKVDNALQSGNSQESVT |
| | | | | EQDSKDSTYSLSSTLTLSKADYEKHKVYACEVTHQGLSSPV |
| | | | | TKSFNRGEC |
| (C-26) | 1-49 | HC | 192 | QVQLVETGGGLVRPGNSLKLSCVTSGFTFSNYRMHWLRQ |
| | | | | PPGKRLEWIAVIKVKSDNYGANYAESVKGRFTISRDDSKSS |
| | | | | VYLQVNRLREEDTATYYCSSPTYPGSSGFAYWGQGTLVTV |
| | | | | SAASTKGPSVFPLAPSSKSTSGGTAALGCLVKDYFPEPVTV |
| | | | | SWNSGALTSGVHTFPAVLQSSGLYSLSSVVTVPSSSLGTQ |
| | | | | TYICNVNHKPSNTKVDKKVEPKSCDKTHTCPPCPAPELVG |
| | | | | GPSVFLLPPKPKDTLMISRTPEVTCVVVDVSHEDPEVKFNW |
| | | | | YVDGVEVHNAKTKPPEEQYNSTLRVVSVLTVLHQDWLNGK |
| | | | | EYKCKVSNKALPAPIEKTISKAKGQPREPQVYTLPPSRDELT |
| | | | | KNQVSLTCLVKGFYPSDIAVEWESNGQPENNYKTTPLVLDS |
| | | | | DGSFFLYSKLTVDKSRWQQGNVFSCSVMHEALHNHYTQK |
| | | | | SLSLSPG |
| | | LC | 189 | DIQMTQTTSSLSASLGDRVTISCRASQDISNYLNWYQQKPD |
| | | | | GTVKLLIYYTSRLHSGVPSRFSGSGSGTDYSLTISNLEQEDI |
| | | | | ATYFCQQGNKFPPTFGGGTKLEINRTVAAPSVFIFPPSDEQ |
| | | | | LKSGTASVVCLLNNFYPREAKVQWKVDNALQSGNSQESVT |
| | | | | EQDSKDSTYSLSSTLTLSKADYEKHKVYACEVTHQGLSSPV |
| | | | | TKSFNRGEC |
| (C-27) | 1-50 | HC | 193 | QVQLVETGGGLVRPGNSLKLSCVTSGFTFSNYRMHWLRQ |
| | | | | PPGKRLEWIAVIKVKSDNYGANYAESVKGRFTISRDDSKSS |
| | | | | VYLQVNRLREEDTATYYCSSPTYPGSSGFAYWGQGTLVTV |
| | | | | SAASTKGPSVFPLAPSSKSTSGGTAALGCLVKDYFPEPVTV |
| | | | | SWNSGALTSGVHTFPAVLQSSGLYSLSSVVTVPSSSLGTQ |
| | | | | TYICNVNHKPSNTKVDKKVEPKSCDKTHTCPPCPAPELVG |
| | | | | GPSVFLLPPKPKDTLMISRTPEVTCVVVDVSHEDPEVKFNW |
| | | | | YVDGVEVHNAKTKPPEEQYNSTLRVVSVLTVLHQDWLNGK |
| | | | | EYKCKVSNWALPAPISKTISKAKGQPREPQVYTLPPSRDEL |
| | | | | TKNQVSLTCLVKGFYPSDIAVEWESNGQPENNYKTTPLVLD |
| | | | | SDGSFFLYSKLTVDKSRWQQGNVFSCSVMHEALHNHYTQ |
| | | | | KSLSLSPG |
| | | LC | 189 | DIQMTQTTSSLSASLGDRVTISCRASQDISNYLNWYQQKPD |
| | | | | GTVKLLIYYTSRLHSGVPSRFSGSGSGTDYSLTISNLEQEDI |
| | | | | ATYFCQQGNKFPPTFGGGTKLEINRTVAAPSVFIFPPSDEQ |
| | | | | LKSGTASVVCLLNNFYPREAKVQWKVDNALQSGNSQESVT |
| | | | | EQDSKDSTYSLSSTLTLSKADYEKHKVYACEVTHQGLSSPV |
| | | | | TKSFNRGEC |

In particularly preferred embodiments, the antibody (or antigen-binding fragment) according to the invention comprises a heavy chain and a light chain, both of which are cumulatively defined by: (i) the above-described options (A-19) and (C-1); or (ii) the above-described options (A-20) and (C-2); or (iii) the above-described options (A-20) and (C-3); or (iv) the above-described options (A-21) and (C-4); or (v) the above-described options (A-22) and (C-5); or (vi) the above-described options (A-22) and (C-6); or (vii) the above-described options (A-25) and (C-7); or (viii) the above-described options (A-23) and (C-8); or (ix) the above-described options (A-23) and (C-9); or (x) the above-described options (A-23) and (C-10); or (xi) the above-described options (A-23) and (C-11); or (xii) the above-described options (A-23) and (C-12); or (xiii) the above-described options (A-22) and (C-13); or (xiv) the above-described options (A-22) and (C-14); or (xv) the above-described options (A-22) and (C-15); or (xvi) the above-described options (A-22) and (C-16); or (xvii) the above-described options (A-22) and (C-17); or (xviii) the above-described options (A-24) and (C-18); or (xix) the above-described options (A-24) and (C-19); or (xx) the above-described options (A-24) and (C-20); or (xxi) the above-described options (A-24) and (C-21); or (xxii) the above-described options (A-24) and (C-22); or (xxiii) the above-described options (A-25) and (C-23); or (xxiv) the above-described options (A-25) and (C-24); or (xxv) the above-described options (A-25) and (C-25); or (xxvi) the above-described options (A-25) and (C-26); or (xxvii) the above-described options (A-25) and (C-27).

In further embodiments, the antibody (or antigen-binding fragment) according to the invention is as defined in any one of the above-described options (C-1) to (C-27), but the respective heavy-chain (HC) sequence additionally has a C-terminal lysine residue, i.e., the glycine (G) at the C-terminus of the respective HC sequence is replaced by glycine-lysine (GK).

The present invention particularly relates to a monoclonal antibody or an antigen-binding fragment thereof, which specifically binds to human CCR8, and which has the CDRs as defined in any one of the above-described options (A-1) to (A-25). Moreover, the invention likewise relates to a monoclonal antibody or an antigen-binding fragment thereof, which specifically binds to human CCR8, and which has a heavy chain variable domain (VH) and a light chain variable domain (VL) as defined in any one of the above-described options (B-1) to (B-28). The invention further relates to a monoclonal antibody or an antigen-binding fragment thereof, which specifically binds to human CCR8, and which has a heavy chain (HC) and a light chain (LC) as defined in any one of the above-described options (C-1) to (C-27).

In further embodiments, the antibody (or antigen-binding fragment) according to the invention binds to the same epitope within human CCR8 as any one of the specific antibodies described in the examples section herein below. In particular, the antibody (or antigen-binding fragment) according to the invention may bind to the same epitope within human CCR8 as an antibody (or antigen-binding fragment) comprising:
(D-1) a heavy chain variable domain (VH) having the amino acid sequence of SEQ ID NO: 82, and a light chain variable domain (VL) having the amino acid sequence of SEQ ID NO: 83; or
(D-2) a heavy chain variable domain (VH) having the amino acid sequence of SEQ ID NO: 84, and a light chain variable domain (VL) having the amino acid sequence of SEQ ID NO: 83; or
(D-3) a heavy chain variable domain (VH) having the amino acid sequence of SEQ ID NO: 85, and a light chain variable domain (VL) having the amino acid sequence of SEQ ID NO: 86; or
(D-4) a heavy chain variable domain (VH) having the amino acid sequence of SEQ ID NO: 87, and a light chain variable domain (VL) having the amino acid sequence of SEQ ID NO: 88; or
(D-5) a heavy chain variable domain (VH) having the amino acid sequence of SEQ ID NO: 89, and a light chain variable domain (VL) having the amino acid sequence of SEQ ID NO: 90; or
(D-6) a heavy chain variable domain (VH) having the amino acid sequence of SEQ ID NO: 91, and a light chain variable domain (VL) having the amino acid sequence of SEQ ID NO: 92; or
(D-7) a heavy chain variable domain (VH) having the amino acid sequence of SEQ ID NO: 93, and a light chain variable domain (VL) having the amino acid sequence of SEQ ID NO: 94; or
(D-8) a heavy chain variable domain (VH) having the amino acid sequence of SEQ ID NO: 95, and a light chain variable domain (VL) having the amino acid sequence of SEQ ID NO: 96; or
(D-9) a heavy chain variable domain (VH) having the amino acid sequence of SEQ ID NO: 97, and a light chain variable domain (VL) having the amino acid sequence of SEQ ID NO: 98; or
(D-10) a heavy chain variable domain (VH) having the amino acid sequence of SEQ ID NO: 99, and a light chain variable domain (VL) having the amino acid sequence of SEQ ID NO: 100; or
(D-11) a heavy chain variable domain (VH) having the amino acid sequence of SEQ ID NO: 101, and a light chain variable domain (VL) having the amino acid sequence of SEQ ID NO: 102; or
(D-12) a heavy chain variable domain (VH) having the amino acid sequence of SEQ ID NO: 103, and a light chain variable domain (VL) having the amino acid sequence of SEQ ID NO: 104; or
(D-13) a heavy chain variable domain (VH) having the amino acid sequence of SEQ ID NO: 105, and a light chain variable domain (VL) having the amino acid sequence of SEQ ID NO: 83; or
(D-14) a heavy chain variable domain (VH) having the amino acid sequence of SEQ ID NO: 106, and a light chain variable domain (VL) having the amino acid sequence of SEQ ID NO: 107; or
(D-15) a heavy chain variable domain (VH) having the amino acid sequence of SEQ ID NO: 108, and a light chain variable domain (VL) having the amino acid sequence of SEQ ID NO: 109; or
(D-16) a heavy chain variable domain (VH) having the amino acid sequence of SEQ ID NO: 110, and a light chain variable domain (VL) having the amino acid sequence of SEQ ID NO: 111; or
(D-17) a heavy chain variable domain (VH) having the amino acid sequence of SEQ ID NO: 112, and a light chain variable domain (VL) having the amino acid sequence of SEQ ID NO: 113; or
(D-18) a heavy chain variable domain (VH) having the amino acid sequence of SEQ ID NO: 114, and a light chain variable domain (VL) having the amino acid sequence of SEQ ID NO: 115; or
(D-19) a heavy chain variable domain (VH) having the amino acid sequence of SEQ ID NO: 138, and a light chain variable domain (VL) having the amino acid sequence of SEQ ID NO: 139; or
(D-20) a heavy chain variable domain (VH) having the amino acid sequence of SEQ ID NO: 140, and a light chain variable domain (VL) having the amino acid sequence of SEQ ID NO: 141; or
(D-21) a heavy chain variable domain (VH) having the amino acid sequence of SEQ ID NO: 140, and a light chain variable domain (VL) having the amino acid sequence of SEQ ID NO: 142; or
(D-22) a heavy chain variable domain (VH) having the amino acid sequence of SEQ ID NO: 143, and a light chain variable domain (VL) having the amino acid sequence of SEQ ID NO: 144; or
(D-23) a heavy chain variable domain (VH) having the amino acid sequence of SEQ ID NO: 145, and a light chain variable domain (VL) having the amino acid sequence of SEQ ID NO: 144; or
(D-24) a heavy chain variable domain (VH) having the amino acid sequence of SEQ ID NO: 146, and a light chain variable domain (VL) having the amino acid sequence of SEQ ID NO: 144; or
(D-25) a heavy chain variable domain (VH) having the amino acid sequence of SEQ ID NO: 147, and a light chain variable domain (VL) having the amino acid sequence of SEQ ID NO: 148; or
(D-26) a heavy chain variable domain (VH) having the amino acid sequence of SEQ ID NO: 149, and a light chain variable domain (VL) having the amino acid sequence of SEQ ID NO: 150; or
(D-27) a heavy chain variable domain (VH) having the amino acid sequence of SEQ ID NO: 151, and a light chain variable domain (VL) having the amino acid sequence of SEQ ID NO: 152; or
(D-28) a heavy chain variable domain (VH) having the amino acid sequence of SEQ ID NO: 153, and a light chain variable domain (VL) having the amino acid sequence of SEQ ID NO: 154.

The specific epitope bound by any of the antibodies (or antigen-binding fragments) referred to in (D-1) to (D-28) above can be identified by any suitable epitope mapping method known in the art (see, e.g., Morris G E (ed.), “Epitope mapping protocols”, Methods in Molecular Biology, Vol. 66, 1996, Humana Press, doi: 10.1385/0896033759; or Opuni K F M et al., Mass Spectrom Rev, 2018, 37(2): 229-241, doi: 10.1002/mas.21516; which are incorporated herein by reference). For example, peptides of varying lengths derived from human CCR8 can be screened for binding to an antibody in order to identify the smallest peptide that can specifically bind to the antibody. A corresponding peptide that specifically binds to the antibody can be identified, e.g., by mass spectrometric analysis. Alternatively, NMR spectroscopy or X-ray crystallography can be used to identify the epitope bound by an antibody. Once identified, the epitopic fragment that binds an antibody of the present invention can be used as an immunogen to obtain further antibodies that specifically bind to the same epitope.

The antibody or antigen-binding fragment according to the invention (including any one of the exemplary or preferred antibodies described herein, such as, e.g., the antibody according to any one of the above-described options (A-1) to (A-25) or any one of the above-described options (B-1) to (B-28) preferably comprises an Fc region, more preferably a human IgG Fc region (e.g., an Fc region of human IgG1, IgG2, IgG3, or IgG4), even more preferably a human IgG1 or IgG4 Fc region, yet even more preferably a human IgG1 Fc region.

The term “Fc region” is well-known in the art and typically relates to a C-terminal region of an immunoglobulin heavy chain that contains at least a portion of the constant region (and that may interact with Fc receptors). In antibodies of the human IgG, IgA or IgD class, the Fc region is typically composed of two identical polypeptide chains which include the second and third constant domains of the antibody's two heavy chains. In antibodies of the human IgM or IgE class, the Fc region is typically composed of two identical polypeptide chains, each of which includes the second, third and fourth heavy-chain constant domains of the respective antibody. The term “Fc region” includes native sequence Fc regions and variant Fc regions. As explained above, the “Fc region” is preferably a human IgG Fc region (e.g., a human IgG1 Fc region), which may extend from the Cys-226 residue (or from the Pro-230 residue) to the C-terminus of each heavy chain. Unless stated otherwise, the numbering of amino acid residues in the Fc region is indicated herein according to the EU numbering system (which is also known as EU index; see, e.g., Kabat E A et al., “Sequences of proteins of immunological interest”, fifth edition, 1991, US Department of Health and Human Services, National Institutes of Health (NIH) publication no. 91-3242).

Notably, the C-terminal lysine residue (Lys-447) of a human IgG (e.g., IgG1) Fc region may be present or may be absent. The cleavage of C-terminal lysine from the Fc region of antibodies is well-known in the art (see, e.g., Harris R J, J Chromatogr A, 1995, 705(1): 129-34, doi: 10.1016/0021-9673(94) 01255-d; Dick L W Jr, Biotechnol Bioeng, 2008, 100(6): 1132-43, doi: 10.1002/bit.21855; Liu H et al., Biotechnol Prog, 2016, 32(5): 1103-12, doi: 10.1002/btpr.2327; or Faid V et al., Eur J Pharm Sci, 2021, 159:105730, doi: 10.1016/j.ejps.2021.105730) and may occur to a greater or lesser extent (or not at all), depending on how the respective antibody is produced. For example, when IgG antibodies are recombinantly produced in CHO cells, the endogenous carboxypeptidases from the CHO cells may cleave the terminal lysine residue from the heavy-chain C-terminus of the IgG antibody, which can result in a heterogenous population of antibodies having a C-terminal lysine residue on each of the two heavy chains, antibodies having only one C-terminal lysine residue (i.e., on only one of the two heavy chains), and antibodies not having any C-terminal lysine residue on the two heavy chains. The production of homogenous populations of antibodies either having or not having a C-terminal lysine residue can be achieved, e.g., by appropriately adjusting the antibody production process, as known in the art. The present invention specifically relates to an antibody (or antigen-binding fragment) as described herein, which has an Fc region (particularly a human IgG Fc region, e.g., a human IgG1 Fc region) that contains a C-terminal lysine residue. Yet, the invention also specifically relates to an antibody (or antigen-binding fragment) as described herein, which has an Fc region (particularly a human IgG Fc region, e.g., a human IgG1 Fc region) that lacks a C-terminal lysine residue, in particular, that does not contain any C-terminal lysine residue. Moreover, the invention also relates to an antibody (or antigen-binding fragment) as described herein, which has an Fc region (particularly a human IgG Fc region, e.g., a human IgG1 Fc region) that lacks the C-terminal glycine-lysine residues (i.e., residues 446 and 447 according to the EU index), particularly an Fc region that does not have any C-terminal glycine-lysine residues.

It is preferred that the antibody or antigen-binding fragment (including any one of the exemplary or preferred antibodies described herein, such as, e.g., the antibody according to any one of the above-described options (A-1) to (A-25) or any one of the above-described options (B-1) to (B-28)) has antibody-dependent cellular cytotoxicity (ADCC) activity, complement-dependent cytotoxicity (CDC) activity, and/or antibody-dependent cellular phagocytosis (ADCP) activity. More preferably, the antibody or antigen-binding fragment has ADCC activity and/or ADCP activity, even more preferably ADCC activity.

As is known in the art, the level of core fucosylation of the Fc region of an antibody (or an antigen-binding fragment) affects its binding to Fcγ receptors (FcγR) on effector cells, particularly FcγRIIIa, and thereby its ADCC activity and/or its ADCP activity. In particular, it has been reported that antibodies having decreased core fucose levels or completely afucosylated antibodies (which do not have any fucose units) exhibit considerably increased ADCC activity via an increased affinity of the (IgG) Fc region for FcγRIIIa on immune cells (see, e.g., Okazaki A et al., J Mol Biol, 2004, 336(5): 1239-49, doi: 10.1016/j.jmb.2004.01.007; Jiang X R et al., Nat Rev Drug Discov, 2011, 10(2): 101-11, doi: 10.1038/nrd3365; WO 03/035835; or EP 1 469 065 A1), and that a decrease or absence of core fucose also promotes ADCP activity mediated by FcγRIIIa-positive monocytes and macrophages (see, e.g., Golay J et al., Blood, 2013, 122(20): 3482-91, doi: 10.1182/blood-2013-05-504043; or Herter S et al., J Immunol, 2014, 192(5): 2252-60, doi: 10.4049/jimmunol. 1301249).

Accordingly, it is preferred that the antibody or antigen-binding fragment according to the invention (including any one of the exemplary or preferred antibodies described herein, such as, e.g., the antibody according to any one of the above-described options (A-1) to (A-25), any one of the above-described options (B-1) to (B-28), or any one of the above-described options (C-1) to (C-27) comprises an Fc region having a reduced core fucose level or having no core fucose units at all. It is particularly preferred that the antibody (or antigen-binding fragment) according to the invention comprises a hypofucosylated or an afucosylated Fc region (particularly an afucosylated human IgG Fc region, more preferably an afucosylated human IgG1 or IgG4 Fc region, even more preferably an afucosylated human IgG1 Fc region). Such hypofucosylated or afucosylated antibodies (or antigen-binding fragments) exhibit increased ADCC, CDC and/or ADCP activity and are therefore particularly effective in depleting CCR8-expressing (CCR8-positive) tumor-infiltrating T regulatory cells.

Antibodies with an Fc region (e.g., a human IgG Fc region, such as IgG1 or IgG4) that lack core fucose or have greatly reduced core fucose levels can be produced by glycoengineering techniques, as known in the art. For example, hypofucosylated or afucosylated antibodies can be obtained by using a production cell line that overexpresses N-acetylglucosaminyltransferase III (GnTIII) in the Golgi apparatus (or, ideally, that overexpresses both GnTIII and Golgi α-mannosidase II (αManII)), which results in the generation of bisected oligosaccharide structures at the Fc region of the antibody and suppresses fucosylation (see, e.g., Ferrara C et al., Biotechnol Bioeng, 2006, 93(5): 851-61, doi: 10.1002/bit.20777). Alternatively, afucosylated antibodies can be obtained, e.g., using a fucosyltransferase-deficient production cell line (e.g., a fucosyltransferase-deficient CHO cell line); such fucosyltransferase-deficient cell lines can be generated, for example, by silencing the expression of α-1,6-fucosyltransferase (FUT8) or by disrupting both FUT8 alleles via homologous recombination (see, e.g., Mori K et al., Biotechnol Bioeng, 2004, 88(7): 901-8, doi: 10.1002/bit.20326; or Yamane-Ohnuki N et al., Biotechnol Bioeng, 2004, 87(5): 614-22, doi: 10.1002/bit.20151). Moreover, cell lines in which the Golgi GDP-fucose transporter gene (Slc35c1) has been inactivated, so as to eliminate fucosylation reactions in the Golgi apparatus, can also be used to produce afucosylated antibodies (see, e.g., Chan K F et al., Biotechnol J, 2016, 11(3): 399-414, doi: 10.1002/biot.201500331). As a further alternative, fucosylation inhibitors, such as 2-fluorofucose or 5-alkynylfucose (particularly 2-deoxy-2-fluoro-L-fucose), can also be used during recombinant expression in order to generate an afucosylated antibody (see, e.g., Okeley N M et al., Proc Natl Acad Sci USA, 2013, 110(14): 5404-9, doi: 10.1073/pnas. 1222263110). Besides the above-mentioned techniques, further approaches to produce afucosylated antibodies (or antigen-binding fragments) have also been described in the literature (see, e.g., Yamane-Ohnuki N et al., MAbs, 2009, 1(3): 230-6, doi: 10.4161/mabs. 1.3.8328; Yu X et al., BioDrugs, 2017, 31(3): 151-166, doi: 10.1007/s40259-017-0223-8; or Pereira N A et al., MAbs, 2018, 10(5): 693-711, doi: 10.1080/19420862.2018.1466767). The present invention thus relates to the production of the antibody or antigen-binding fragment according to the invention, using a production cell line for hypofucosylation or afucosylation (i.e., a host cell for hypofucosylation or afucosylation), such as, e.g., (i) a production cell line (or host cell) that overexpresses N-acetylglucosaminyltransferase III (GnTIII) in the Golgi apparatus (preferably that overexpresses both N-acetylglucosaminyltransferase III (GnTIII) and Golgi α-mannosidase II (αManII) in the Golgi apparatus), (ii) a fucosyltransferase-deficient production cell line (or host cell), (iii) a production cell line (or host cell) wherein the Golgi GDP-fucose transporter gene (Slc35c1) is inactivated, or (iv) a production cell line (or host cell) exposed to (or incubated with) one or more fucosylation inhibitors (e.g., 2-fluorofucose or 5-alkynylfucose, particularly 2-deoxy-2-fluoro-L-fucose); the invention likewise relates to an antibody or antigen-binding fragment obtainable (or obtained) by using such a production cell line (or host cell).

It is furthermore preferred that the antibody or antigen-binding fragment according to the invention (including any one of the exemplary or preferred antibodies described herein, such as, e.g., the antibody according to any one of the above-described options (A-1) to (A-25) or any one of the above-described options (B-1) to (B-28)) comprises an Fc region (as described herein above) having one or more mutations enhancing ADCC activity, CDC activity and/or ADCP activity. Such mutations have been described in the literature (see, e.g., Wang X et al., Protein Cell, 2018, 9(1): 63-73, doi: 10.1007/s13238-017-0473-8; Chiu M L et al., Antibodies (Basel), 2019, 8(4): 55, doi: 10.3390/antib8040055; or Liu R et al., Antibodies (Basel), 2020, 9(4): 64, doi: 10.3390/antib9040064). For instance, an antibody (or antigen-binding fragment) comprising a human IgG1 Fc region can be used, which has one or more (preferably two or more, three or more, four or more, five or more, six or more, seven or more, or even all) mutations selected from S132I, L142P, A162V, S166N, S219Y, K222N, H224L, T225S, P227S, P232S, E233D (or E233G), L235V, G236A, S239D, V2401, F241L, F243L (or F243I), K246T (or K246I), P247H (or P247L), K248M, L251F, R255L (or R255Q), E258D (or E258G), H268D, D270E, F275Y, V279L, V281M, V282M, V284A, G285E, K288N (or K288M), K290E (or K290T), P291S, R292P (or R292L or R292G), S298A (or S298N), Y300L, S304G, V305I, E308D, N315I, K317N, E318K (or E318D), Y319F, K320E, K326E (or K326N or K326W), A330L (or A330S), 1332E, E333A (or E333S), K334A (or K334E or K334N or K334I), A339V, Q347H, M352L, P353Q, T359N, T366S (or T366N), K370N, G371D, F372Y (or F372L), S375C, 1377F (or 1377N), V379L (or V379M), E380D, W381R, N384K, G385E, E389G, K392R, T394M, P395S, P396L (or P396H), V397M, L398V (or L398Q), S400P, D401V, S4071 (or S407R), K409R, K414N, S415I, N421K, and S440N, particularly from L235V, G236A, S239D, F243L, R292P, S298A, Y300L, V305I, A330L, 1332E, E333A, K334A, 1332E, and P396L (numbering according to EU index; including, e.g., the combinations S239D/1332E, S239D/A330L/1332E, G236A/S239D/1332E, F243L/R292P/Y300L, F243L/R292P/Y300L/P396L, F243L/R292P/Y300L/V305I/P396L, L235V/F243L/R292P/Y300L/P396L, L235V/F243L/R292P/Y300L/K326W/E333S/P396L, S239D/F243L/R292P/Y300L/V305I/A330L/1332E/P396L, or S298A/E333A/K334A); such mutations result in an enhanced interaction with human FcγRIIIa and, thus, an enhanced ADCC activity and/or ADCP activity (see, e.g., Lazar G A et al., Proc Natl Acad Sci USA, 2006, 103(11): 4005-10, doi: 10.1073/pnas.0508123103; Stavenhagen J B et al., Cancer Res, 2007, 67(18): 8882-90, doi: 10.1158/0008-5472.CAN-07-0696; or WO 2004/063351). Also an antibody (or antigen-binding fragment) comprising a human IgG1 Fc region can be used, which has one or more of the mutations (including any of the combinations of mutations) disclosed in WO 2004/063351, particularly in Table 2, 3 or 4 of WO 2004/063351 (which is incorporated herein by reference in its entirety). Moreover, an antibody (or antigen-binding fragment) comprising a human IgG1 Fc region can be used, which has one or more (preferably two or more, three or more, four or more, or even all) mutations selected from S267E, H268F, S324T, K326A (or K326W), E333A, and E345R (numbering according to EU index; including, e.g., the combinations K326A/E333A, K326W/E333A, H268F/S324T, S267E/H268F, S267E/S324T, E345R, or S267E/H268F/S324T); such mutations result in an enhanced CDC activity. It is particularly preferred that an antibody (or antigen-binding fragment) according to the invention comprises a human IgG1 Fc region having any of the following mutations: (i) S239D/1332E; (ii) S239D/A330L/1332E; (iii) L235V/F243L/R292P/Y300L/P396L; or (iv) L235V/F243L/R292P/Y300L/K326W/E333S/P396L (numbering according to EU index). Even more preferably, an antibody (or antigen-binding fragment) according to the invention comprises a human IgG1 Fc region having the S239D/1332E mutation (numbering according to EU index).

It is preferred that the antibody or antigen-binding fragment according to the invention (including any one of the exemplary or preferred antibodies described herein, such as, e.g., the antibody according to any one of the above-described options (A-1) to (A-25) or any one of the above-described options (B-1) to (B-28) has a depleting activity against CCR8-positive immune cells.

It is furthermore preferred that the antibody or antigen-binding fragment according to the invention (including any one of the exemplary or preferred antibodies described herein, such as, e.g., the antibody according to any one of the above-described options (A-1) to (A-25) or any one of the above-described options (B-1) to (B-28)) has one or more (preferably all) of the following cellular activities:
depleting CCR8-positive cells (preferably CCR8-positive T cells and/or CCR8-positive macrophages, more preferably CCR8-positive T regulatory cells and/or CCR8-positive natural killer T cells (NKT cells); even more preferably CCR8-positive T regulatory cells; still more preferably CCR8-positive tumor-infiltrating T regulatory cells);
inhibiting the CCL1-induced migration of CCR8-positive cells (preferably CCR8-positive T cells and/or CCR8-positive macrophages, more preferably CCR8-positive T regulatory cells and/or CCR8-positive natural killer T cells (NKT cells); even more preferably CCR8-positive T regulatory cells); and/or
inhibiting the CCL1-induced activation of CCR8-positive cells (preferably CCR8-positive T cells and/or CCR8-positive macrophages, more preferably CCR8-positive T regulatory cells and/or CCR8-positive natural killer T cells (NKT cells); even more preferably CCR8-positive T regulatory cells).

These cellular activities can be assessed using any suitable assay, e.g., as described in the examples herein below and/or in the literature.

Thus, it is preferred that the antibody or antigen-binding fragment according to the invention (including any one of the exemplary or preferred antibodies described herein) has an activity of depleting CCR8-positive cells (i.e., depletes CCR8-positive cells), preferably an activity of depleting CCR8-positive T cells and/or CCR8-positive macrophages, more preferably an activity of depleting CCR8-positive T regulatory cells and/or CCR8-positive natural killer T cells (NKT cells), even more preferably an activity of depleting CCR8-positive T regulatory cells (particularly CCR8-positive tumor-infiltrating T regulatory cells).

It is furthermore preferred that the antibody or antigen-binding fragment according to the invention (including any one of the exemplary or preferred antibodies described herein) has an activity of inhibiting the CCL1-induced migration of CCR8-positive cells (i.e., inhibits the CCL1-induced migration of CCR8-positive cells), preferably an activity of inhibiting the CCL1-induced migration of CCR8-positive T cells and/or CCR8-positive macrophages, more preferably an activity of inhibiting the CCL1-induced migration of CCR8-positive T regulatory cells and/or CCR8-positive natural killer T cells (NKT cells), even more preferably an activity of inhibiting the CCL1-induced migration of CCR8-positive T regulatory cells.

Moreover, it is preferred that the antibody or antigen-binding fragment according to the invention (including any one of the exemplary or preferred antibodies described herein) has an activity of inhibiting the CCL1-induced activation of CCR8-positive cells (i.e., inhibits the CCL1-induced activation of CCR8-positive cells), preferably an activity of inhibiting the CCL1-induced activation of CCR8-positive T cells and/or CCR8-positive macrophages, more preferably an activity of inhibiting the CCL1-induced activation of CCR8-positive T regulatory cells and/or CCR8-positive natural killer T cells (NKT cells), even more preferably an activity of inhibiting the CCL1-induced activation of CCR8-positive T regulatory cells.

It is also preferred that the antibody or antigen-binding fragment according to the invention (including any one of the exemplary or preferred antibodies described herein) has an activity of inhibiting the CCL1-induced internalization of CCR8.

Furthermore, it is preferred that the antibody or antigen-binding fragment according to the invention (including any one of the exemplary or preferred antibodies described herein, such as, e.g., the antibody according to any one of the above-described options (A-1) to (A-25) or any one of the above-described options (B-1) to (B-28)) has a depleting activity against CCR8-positive cancer cells. Preferably, said CCR8-positive cancer cells are from lymphoma, more preferably from cutaneous T-cell lymphoma, even more preferably from Sézary syndrome.

It is further preferred that the antibody or antigen-binding fragment according to the invention does not bind to peripheral immune cells from a healthy donor. In particular, it is preferred that the antibody or antigen-binding fragment does not bind to peripheral immune cells extracted from PBMCs from a healthy donor. This can be assessed by flow cytometry, e.g., using the protocol described in Example 12.

The present invention also relates to the antibody or antigen-binding fragment provided herein (including any one of the exemplary or preferred antibodies or antigen-binding fragment described herein, such as, e.g., those according to any one of the above-described options (A-1) to (A-25) or any one of the above-described options (B-1) to (B-28), wherein the antibody or antigen-binding fragment is incorporated into a chimeric antigen receptor (CAR). Chimeric antigen receptors (CARs) typically combine antigen-binding and immune cell (e.g., T cell or NK cell) activating functions into a single receptor and can be used for CAR cell therapy (particularly for CAR T cell therapy, including CAR alpha-beta-T cell therapy or CAR gamma-delta-T cell therapy, or for CAR NK cell therapy); see, e.g., Yong C S M et al., Immunol Cell Biol, 2017, 95(4): 356-63, doi: 10.1038/icb.2016.128; Newick K et al., Annu Rev Med, 2017, 68: 139-52, doi: 10.1146/annurev-med-062315-120245; Miliotou A N et al., Curr Pharm Biotechnol, 2018,19(1): 5-18, doi: 10.2174/1389201019666180418095526; Martinez M et al., Front Immunol, 2019, 10: 128, doi: 10.3389/fimmu.2019.00128; Ahmad A, Int J Mol Sci, 2020, 21(12): 4303, doi: 10.3390/ijms21124303; Hong M et al., Cancer Cell, 2020, 38(4): 473-88, doi: 10.1016/j.ccell.2020.07.005; Xie G et al., EBioMedicine, 2020, 59: 102975, doi: 10.1016/j.ebiom.2020.102975; Khawar M B et al., Front Immunol, 2021, 12: 707542, doi: 10.3389/fimmu.2021.707542; Qin V M et al., Cancers (Basel), 2021, 13(3): 404, doi: 10.3390/cancers13030404; Saura-Esteller J et al., Front Immunol, 2022, 13: 915837, doi: 10.3389/fimmu.2022.915837; or Zhang L et al., Biomark Res, 2022, 10(1): 12, doi: 10.1186/s40364-022-00364-6 (wherein each of the aforementioned publications is incorporated herein by reference). CARs are typically composed of four regions, i.e., (i) an antigen recognition domain (which may be, e.g., a single-chain variable fragment (scFv) or a single-domain antibody), (ii) an extracellular hinge region, (iii) a transmembrane domain, and (iv) an intracellular immune cell signaling domain. The intracellular immune cell signaling domain is preferably an intracellular T cell signaling domain (particularly an intracellular alpha-beta T cell signaling domain; e.g., a CD3-zeta cytoplasmic domain, which may be combined with one or more co-stimulatory domains, such as, e.g., co-stimulatory domains derived from CD28, 4-1BB, CD27, OX40, ICOS, or any combination thereof), an intracellular gamma-delta-T cell signaling domain (e.g., a CD3-zeta cytoplasmic domain, which may be combined with one or more co-stimulatory domains, such as, e.g., co-stimulatory domains derived from CD28, 4-1BB, CD27, OX40, ICOS, or any combination thereof), or an intracellular natural killer cell (NK cell) signaling domain (e.g., a CD3-zeta cytoplasmic domain, a DAP10 cytoplasmic domain and/or a DAP12 cytoplasmic domain, any of which may be combined with one or more co-stimulatory domains, such as, e.g., co-stimulatory domains derived from CD28, 4-1BB, 2B4, CD27, OX40, ICOS, or any combination thereof). The present invention thus also provides a chimeric antigen receptor (CAR) comprising an antibody or antigen-binding fragment according to the invention. In particular, the present invention provides a chimeric antigen receptor (CAR) comprising (or, preferably, consisting of) the following moieties (preferably in this order): an antigen recognition domain which is an antibody or antigen-binding fragment according to the invention (e.g., a single-chain variable fragment (scFv) or a single-domain antibody, preferably a single-chain variable fragment which is defined in accordance with any one of the above-described options (A-1) to (A-25) or any one of the above-described options (B-1) to (B-28); an extracellular hinge region; a transmembrane domain; and an intracellular signaling domain (particularly an intracellular immune cell signaling domain, such as an intracellular T cell or NK cell signaling domain; e.g., a CD3-zeta cytoplasmic domain, preferably a CD3-zeta cytoplasmic domain combined with one or more co-stimulatory domains, such as, e.g., CD28, 4-1BB, CD27, OX40, ICOS or any combination thereof). The invention further relates to an immune cell (e.g., a T cell (including, e.g., an alpha-beta-T cell or a gamma-delta-T cell) or a natural killer (NK) cell, preferably a T cell) expressing a chimeric antigen receptor (CAR) according to the invention. Such cells can be produced, e.g., by obtaining immune cells (e.g., T cells or NK cells) from the blood of the subject/patient to be treated or from the blood of a healthy donor (who should be of the same species as the subject/patient to be treated, and is preferably a human), and by modifying said immune cells to express a CAR according to the invention. The present invention also relates to therapeutic applications of such CARs and of immune cells (particularly T cells or NK cells) expressing such CARs, particularly their use in the treatment of cancer. Accordingly, the present invention relates to the same therapeutic uses and the same methods of treatment as described herein in connection with the antibody or antigen-binding fragment according to the invention, wherein said uses or said methods comprise the administration of an immune cell (preferably a T cell (e.g., an alpha-beta-T cell or a gamma-delta-T cell) or an NK cell) expressing a chimeric antigen receptor (CAR) according to the invention (instead of the administration of the antibody or antigen-binding fragment according to the invention).

The present invention further relates to the antibody or antigen-binding fragment provided herein (including any one of the exemplary or preferred antibodies or antigen-binding fragment described herein, such as, e.g., those according to any one of the above-described options (A-1) to (A-25), any one of the above-described options (B-1) to (B-28), or any one of the above-described options (C-1) to (C-27)), wherein the antibody or antigen-binding fragment is incorporated into an antibody-drug conjugate (ADC). Thus, the present invention provides an antibody-drug conjugate comprising (preferably consisting of) an antibody or antigen-binding fragment according to the invention, a linker, and a drug. The preparation of antibody-drug conjugates is well-known in the art and has been described, e.g., in: Casi G et al., J Control Release, 2012, 161(2): 422-8, doi: 10.1016/j.jconrel.2012.01.026; Zolot R S et al., Nat Rev Drug Discov, 2013, 12(4): 259-60, doi: 10.1038/nrd3980; Perez H L et al., Drug Discov Today, 2014, 19(7): 869-81, doi: 10.1016/j.drudis.2013.11.004; Thomas A et al., Lancet Oncol, 2016, 17(6): e254-e262, doi: 10.1016/S1470-2045(16) 30030-4; Beck A et al., Nat Rev Drug Discov, 2017, 16(5): 315-337, doi: 10.1038/nrd.2016.268; Chau C H et al., Lancet, 2019, 394(10200): 793-804, doi: 10.1016/S0140-6736(19) 31774-X; Joubert N et al., Pharmaceuticals (Basel), 2020, 13(9): 245, doi: 10.3390/ph13090245; Khongorzul P et al., Mol Cancer Res, 2020, 18(1): 3-19, doi: 10.1158/1541-7786.MCR-19-0582; Baah S et al., Molecules, 2021, 26(10): 2943, doi: 10.3390/molecules26102943; or Drago J Z et al., Nat Rev Clin Oncol, 2021, 18(6): 327-344, doi: 10.1038/s41571-021-00470-8 (all of which are incorporated herein by reference in their entirety). The drug comprised in an antibody-drug conjugate according to the present invention is preferably an anticancer drug (such as, e.g., any of the anticancer drugs described herein, particularly as described in the context of combination treatments), more preferably a cytotoxic drug (or cytotoxic anticancer drug). It will be understood that a prodrug (or precursor) of a cytotoxic drug (or cytotoxic anticancer drug) can also be used as the drug comprised in an antibody-drug conjugate according to the invention. A number of drugs for ADCs are known in the art and can be used for the antibody-drug conjugate according to the invention, including any of the drugs mentioned in any of the documents referenced in this paragraph. In particular, the drug may be, e.g., a microtubulin inhibitor (such as, e.g., monomethyl auristatin A (MMAE), monomethyl auristatin F (MMAF), or mertansine), a DNA binder (such as, e.g., calicheamicin), a topoisomerase 1 inhibitor (such as, e.g., SN-38 or exatecan), or a glucocorticoid receptor modulator (GRM; such as, e.g., dexamethasone, budesonide, or any of the GRMs referred to in Hobson A D et al., J Med Chem, 2022, 65(6): 4500-4533, doi: 10.1021/acs.jmedchem. 1c02099, which is incorporated herein by reference). In the antibody-drug conjugate, the linker couples the antibody (or antigen-binding fragment) to the drug. The linker may be cleavable or non-cleavable. For example, the linker may be a cleavable linker containing a disulfide linkage, a hydrazone linkage, or a peptide linkage, or it may be a non-cleavable linker containing a thioether linkage. Suitable linkers are known in the art and have been described, e.g., in: Nolting B, Methods Mol Biol, 2013, 1045:71-100, doi: 10.1007/978-1-62703-541-5_5; Lu J et al., Int J Mol Sci, 2016, 17(4): 561, doi: 10.3390/ijms17040561; Tsuchikama K et al., Protein Cell, 2018, 9(1): 33-46, doi: 10.1007/s13238-016-0323-0; Bargh J D et al., Chem Soc Rev, 2019, 48(16): 4361-4374, doi: 10.1039/c8cs00676h; Bargh J D et al., Chem Sci, 2020, 11(9): 2375-2380, doi: 10.1039/c9sc06410a; Bargh J D et al., Chem Commun (Camb), 2021, 57(28): 3457-3460, doi: 10.1039/d1cc00957e; Su Z et al., Acta Pharm Sin B, 2021, 11(12): 3889-3907, doi: 10.1016/j.apsb.2021.03.042; Sheyi R et al., Pharmaceutics, 2022, 14(2): 396, doi: 10.3390/pharmaceutics14020396; or Bulger P G et al., Org Process Res Dev, 2023, 27(7): 1248-1257, doi: 10.1021/acs.oprd.3c00136 (all of which are incorporated herein by reference in their entirety); any of the linkers mentioned in any of these documents or in any of the other documents referenced in this paragraph can be used as a linker of an antibody-drug conjugate according to the present invention.

The antibodies and antigen-binding fragments according to the present invention may be prepared by a variety of techniques routinely used in the art. For example, antibodies can be prepared by immunizing a non-human animal with an antigen of interest (e.g., a CCR8 peptide as described herein above or in Example 1) and subsequently isolating antigen-reactive antibody-producing B-cells. To this end, human CCR8 or a partial sequence thereof, e.g., any of the CCR8 peptides described herein, can be used to identify positive clones, i.e., to identify clones producing antibodies that specifically bind to human CCR8 or the respective partial sequence (or the respective CCR8 peptide). Methods of isolating and selecting clones that produce antibodies having desired characteristics are well-known in the art. For example, for immunization of a non-human animal, the immunogen used (e.g., a CCR8 peptide) may be coupled to an adjuvant-carrier (such as, e.g., keyhole limpet hemocyanin, KLH) and/or may be administered together with an adjuvant composition (such as, e.g., Freund's complete adjuvant or Freund's incomplete adjuvant) to improve immunogenicity. Animals can be immunized according to a standard schedule, such as weekly, monthly or a combination of weekly and monthly immunizations, depending on the animal, the antigen and the antibody titer. To determine the immune response of the animals, antibody titer in serum can be tested according to standard procedures. The peripheral blood mononuclear cell (PBMC) fraction of positive animals can be isolated and antigen-reactive B-cells can be purified using standard techniques, such as, e.g., ELISA or column-based techniques to purify reactive B-cells from serum (see, e.g., Seeber S et al., PLOS One, 2014, 9(2): e86184, doi: 10.1371/journal.pone.0086184). Clones binding the antigen of interest can then be selected for subsequent recombinant processing.

Other examples of methods suitable for producing or isolating antibodies and antibody antigen-binding fragments according to the invention include methods that select a recombinant antibody from a peptide or protein library (e.g., a bacteriophage, ribosome, oligonucleotide, RNA, cDNA, or yeast display library) using binding activities of interest. For example, antibodies or antigen-binding fragments can be selected from such libraries by selecting for specific binding to human CCR8 or a partial sequence thereof (e.g., any of the CCR8 peptides described herein, including those described in Example 1). Corresponding display libraries are well-known in the art and are available from various commercial vendors including, e.g., MorphoSys (Planegg, Germany) or Bioinvent (Lund, Sweden). Selected clones can then be processed according to routine methods for subsequent recombinant processing.

Accordingly, the present invention also provides a nucleic acid molecule encoding an anti-CCR8 antibody (or antigen-binding fragment) described herein, including also a nucleic acid molecule encoding an anti-CCR8 heavy chain and/or light chain variable domain as disclosed herein. In particular, the invention relates to one or more nucleic acid molecules encoding the heavy chain and the light chain of the antibody or antigen-binding fragment provided herein. In this regard, the term “nucleic acid molecule” (or synonymous terms such as “nucleic acid”, “nucleic acid sequence”, or “polynucleotide”) includes genomic DNA, cDNA or RNA (e.g., mRNA) capable of driving expression of an antibody or antigen-binding fragment of the invention. The nucleic acid molecules of the invention may be of natural or of synthetic or semi-synthetic origin. Thus, for example, the nucleic acid molecules may be synthesized according to conventional protocols of organic chemistry, according to recombinant methods, or produced semi-synthetically, e.g., by combining chemical synthesis and recombinant methods.

In particular, the invention provides a nucleic acid (or polynucleotide) encoding:
(1) a heavy chain variable domain (VH) having the sequence of SEQ ID NO: 82 and/or a light chain variable domain (VL) having the sequence of SEQ ID NO: 83; or
(2) a heavy chain variable domain (VH) having the sequence of SEQ ID NO: 84 and/or a light chain variable domain (VL) having the sequence of SEQ ID NO: 83; or
(3) a heavy chain variable domain (VH) having the sequence of SEQ ID NO: 85 and/or a light chain variable domain (VL) having the sequence of SEQ ID NO: 86; or
(4) a heavy chain variable domain (VH) having the sequence of SEQ ID NO: 87 and/or a light chain variable domain (VL) having the sequence of SEQ ID NO: 88; or
(5) a heavy chain variable domain (VH) having the sequence of SEQ ID NO: 89 and/or a light chain variable domain (VL) having the sequence of SEQ ID NO: 90; or
(6) a heavy chain variable domain (VH) having the sequence of SEQ ID NO: 91 and/or a light chain variable domain (VL) having the sequence of SEQ ID NO: 92; or
(7) a heavy chain variable domain (VH) having the sequence of SEQ ID NO: 93 and/or a light chain variable domain (VL) having the sequence of SEQ ID NO: 94; or
(8) a heavy chain variable domain (VH) having the sequence of SEQ ID NO: 95 and/or a light chain variable domain (VL) having the sequence of SEQ ID NO: 96; or
(9) a heavy chain variable domain (VH) having the sequence of SEQ ID NO: 97 and/or a light chain variable domain (VL) having the sequence of SEQ ID NO: 98; or
(10) a heavy chain variable domain (VH) having the sequence of SEQ ID NO: 99 and/or a light chain variable domain (VL) having the sequence of SEQ ID NO: 100; or
(11) a heavy chain variable domain (VH) having the sequence of SEQ ID NO: 101 and/or a light chain variable domain (VL) having the sequence of SEQ ID NO: 102; or
(12) a heavy chain variable domain (VH) having the sequence of SEQ ID NO: 103 and/or a light chain variable domain (VL) having the sequence of SEQ ID NO: 104; or
(13) a heavy chain variable domain (VH) having the sequence of SEQ ID NO: 105 and/or a light chain variable domain (VL) having the sequence of SEQ ID NO: 83; or
(14) a heavy chain variable domain (VH) having the sequence of SEQ ID NO: 106 and/or a light chain variable domain (VL) having the sequence of SEQ ID NO: 107; or
(15) a heavy chain variable domain (VH) having the sequence of SEQ ID NO: 108 and/or a light chain variable domain (VL) having the sequence of SEQ ID NO: 109; or
(16) a heavy chain variable domain (VH) having the sequence of SEQ ID NO: 110 and/or a light chain variable domain (VL) having the sequence of SEQ ID NO: 111; or
(17) a heavy chain variable domain (VH) having the sequence of SEQ ID NO: 112 and/or a light chain variable domain (VL) having the sequence of SEQ ID NO: 113; or
(18) a heavy chain variable domain (VH) having the sequence of SEQ ID NO: 114 and/or a light chain variable domain (VL) having the sequence of SEQ ID NO: 115; or
(19) a heavy chain variable domain (VH) having the sequence of SEQ ID NO: 138 and/or a light chain variable domain (VL) having the sequence of SEQ ID NO: 139; or
(20) a heavy chain variable domain (VH) having the sequence of SEQ ID NO: 140 and/or a light chain variable domain (VL) having the sequence of SEQ ID NO: 141; or
(21) a heavy chain variable domain (VH) having the sequence of SEQ ID NO: 140 and/or a light chain variable domain (VL) having the sequence of SEQ ID NO: 142; or
(22) a heavy chain variable domain (VH) having the sequence of SEQ ID NO: 143 and/or a light chain variable domain (VL) having the sequence of SEQ ID NO: 144; or
(23) a heavy chain variable domain (VH) having the sequence of SEQ ID NO: 145 and/or a light chain variable domain (VL) having the sequence of SEQ ID NO: 144; or
(24) a heavy chain variable domain (VH) having the sequence of SEQ ID NO: 146 and/or a light chain variable domain (VL) having the sequence of SEQ ID NO: 132; or
(25) a heavy chain variable domain (VH) having the sequence of SEQ ID NO: 147 and/or a light chain variable domain (VL) having the sequence of SEQ ID NO: 148; or
(26) a heavy chain variable domain (VH) having the sequence of SEQ ID NO: 149 and/or a light chain variable domain (VL) having the sequence of SEQ ID NO: 150; or
(27) a heavy chain variable domain (VH) having the sequence of SEQ ID NO: 151 and/or a light chain variable domain (VL) having the sequence of SEQ ID NO: 152; or
(28) a heavy chain variable domain (VH) having the sequence of SEQ ID NO: 153 and/or a light chain variable domain (VL) having the sequence of SEQ ID NO: 154; or
(29) a heavy chain (HC) having the sequence of SEQ ID NO: 159 and/or a light chain (LC) having the sequence of SEQ ID NO: 160; or
(30) a heavy chain (HC) having the sequence of SEQ ID NO: 161 and/or a light chain (LC) having the sequence of SEQ ID NO: 162; or
(31) a heavy chain (HC) having the sequence of SEQ ID NO: 163 and/or a light chain (LC) having the sequence of SEQ ID NO: 162; or
(32) a heavy chain (HC) having the sequence of SEQ ID NO: 161 and/or a light chain (LC) having the sequence of SEQ ID NO: 164; or
(33) a heavy chain (HC) having the sequence of SEQ ID NO: 165 and/or a light chain (LC) having the sequence of SEQ ID NO: 166; or
(34) a heavy chain (HC) having the sequence of SEQ ID NO: 167 and/or a light chain (LC) having the sequence of SEQ ID NO: 166; or
(35) a heavy chain (HC) having the sequence of SEQ ID NO: 168 and/or a light chain (LC) having the sequence of SEQ ID NO: 169; or
(36) a heavy chain (HC) having the sequence of SEQ ID NO: 170 and/or a light chain (LC) having the sequence of SEQ ID NO: 171; or
(37) a heavy chain (HC) having the sequence of SEQ ID NO: 172 and/or a light chain (LC) having the sequence of SEQ ID NO: 171; or
(38) a heavy chain (HC) having the sequence of SEQ ID NO: 173 and/or a light chain (LC) having the sequence of SEQ ID NO: 171; or
(39) a heavy chain (HC) having the sequence of SEQ ID NO: 174 and/or a light chain (LC) having the sequence of SEQ ID NO: 171; or
(40) a heavy chain (HC) having the sequence of SEQ ID NO: 175 and/or a light chain (LC) having the sequence of SEQ ID NO: 171; or
(41) a heavy chain (HC) having the sequence of SEQ ID NO: 176 and/or a light chain (LC) having the sequence of SEQ ID NO: 177; or
(42) a heavy chain (HC) having the sequence of SEQ ID NO: 178 and/or a light chain (LC) having the sequence of SEQ ID NO: 177; or
(43) a heavy chain (HC) having the sequence of SEQ ID NO: 179 and/or a light chain (LC) having the sequence of SEQ ID NO: 177; or
(44) a heavy chain (HC) having the sequence of SEQ ID NO: 180 and/or a light chain (LC) having the sequence of SEQ ID NO: 177; or
(45) a heavy chain (HC) having the sequence of SEQ ID NO: 181 and/or a light chain (LC) having the sequence of SEQ ID NO: 177; or
(46) a heavy chain (HC) having the sequence of SEQ ID NO: 182 and/or a light chain (LC) having the sequence of SEQ ID NO: 183; or
(47) a heavy chain (HC) having the sequence of SEQ ID NO: 184 and/or a light chain (LC) having the sequence of SEQ ID NO: 183; or
(48) a heavy chain (HC) having the sequence of SEQ ID NO: 185 and/or a light chain (LC) having the sequence of SEQ ID NO: 183; or
(49) a heavy chain (HC) having the sequence of SEQ ID NO: 186 and/or a light chain (LC) having the sequence of SEQ ID NO: 183; or
(50) a heavy chain (HC) having the sequence of SEQ ID NO: 187 and/or a light chain (LC) having the sequence of SEQ ID NO: 183; or
(51) a heavy chain (HC) having the sequence of SEQ ID NO: 188 and/or a light chain (LC) having the sequence of SEQ ID NO: 189; or
(52) a heavy chain (HC) having the sequence of SEQ ID NO: 190 and/or a light chain (LC) having the sequence of SEQ ID NO: 189; or
(53) a heavy chain (HC) having the sequence of SEQ ID NO: 191 and/or a light chain (LC) having the sequence of SEQ ID NO: 189; or
(54) a heavy chain (HC) having the sequence of SEQ ID NO: 192 and/or a light chain (LC) having the sequence of SEQ ID NO: 189; or
(55) a heavy chain (HC) having the sequence of SEQ ID NO: 193 and/or a light chain (LC) having the sequence of SEQ ID NO: 189.

The present invention also provides vectors comprising nucleic acid molecules encoding the antibodies or antigen-binding fragments of the invention. As used herein, the term “vector” relates, in particular, to a circular or linear nucleic acid molecule that can autonomously replicate in a host cell into which it has been introduced. Examples of vectors suitable to be used in the present invention include cosmids, plasmids (e.g., naked plasmids, or plasmids contained in liposomes), viruses (e.g., lentiviruses, retroviruses, adenoviruses, or adeno-associated viruses), or bacteriophages. Various further suitable vectors are known in the art. Suitable vectors and their use are also described, e.g., in Green M R et al., “Molecular Cloning: A Laboratory Manual”, fourth edition, 2012, Cold Spring Harbor Laboratory Press. It will be understood that further nucleic acid sequences may be present in the vectors, e.g., sequences controlling vector replication and/or the expression of the desired sequence(s) in the host cell system. For example, the vectors may comprise the nucleic acid molecule encoding an antibody or antigen-binding fragment of the invention operably linked to, and/or under the control of, one or more regulatory sequences. Such regulatory sequences include DNA sequences that promote (or are necessary for) the expression of coding sequences to which they are operably linked. The vectors may also contain control sequences affecting, e.g., expression and/or replication. Such regulatory and control sequences can be appropriately chosen depending on the host cell to be used. For example, in prokaryotes, control sequences typically include promoters, ribosomal binding sites, and terminators. In eukaryotes, control sequences typically include promoters, terminators and, in some instances, enhancers, transactivators and/or transcription factors.

The vectors according to the present invention are preferably expression vectors. An expression vector is capable of directing the replication and the expression of a nucleic acid molecule of the invention in a host cell and, thus, may provide for the expression of, e.g., the heavy chain and/or light chain variable domains of any one of the antibodies described herein. In some embodiments, a vector may comprise further sequences to ensure that not only the heavy and light chain variable domains are expressed, but also the remaining heavy and light chain constant regions, such that a full-length antibody (e.g., a full-length IgG antibody) is expressed. Suitable expression vectors have been described in the literature can be readily determined for any particular cell system using routine methods. Preferably, the vectors provided herein comprise a polynucleotide encoding an antibody or antigen-binding fragment according to the invention, which is operably linked to one or more regulatory or control sequences. The vectors provided herein preferably further comprise a promoter. The vectors may also comprise a selection marker gene, a replication origin, and/or a termination signal for transcription. Expression vectors can be used to drive transient or constitutive expression in a host cell.

The nucleic acid molecules and/or vectors of the invention can be designed for transfection of prokaryotic or eukaryotic host cells by any methods known in the art or described herein. Examples of such methods include chemical-based methods (e.g., using polyethyleneimine, calcium phosphate, liposomes, or DEAE-dextran), nonchemical methods (e.g., electroporation, nucleofection, sonoporation, optical transfection, gene electrotransfer, or hydrodynamic delivery), particle-based methods (e.g., gene gun, magnetofection, or impalefection), phage vector-based methods, or viral methods. For example, expression vectors derived from viruses such as retroviruses, vaccinia virus, adeno-associated viruses, herpes viruses, Semliki Forest virus, or bovine papilloma virus may be used for transfection of the nucleic acid molecules into a targeted cell population. Moreover, baculoviral systems can also be used as vector in eukaryotic expression systems.

Prokaryotic hosts include all bacteria which can be transformed, transduced or transfected with DNA or RNA molecules for the expression of a protein. In particular, prokaryotic hosts may include gram-negative as well as gram-positive bacteria such as, e.g., E. coli, S. typhimurium, Serratia marcescens, Corynebacterium spp. (e.g., Corynebacterium glutamicum), Pseudomonas spp. (Pseudomonas fluorescens), Lactobacillus spp., Streptomyces spp., Salmonella spp., or Bacillus subtilis. Eukaryotic hosts include, e.g., yeast, higher plant, insect and mammalian cells. Examples of suitable mammalian host cells include, in particular, Hela cells, HEK293 cells, H9 cells, Per.C6 cells, Jurkat cells, mouse NIH3T3 cells, NS/0 cells, SP2/0 cells, C127 cells, COS cells (e.g., COS 1 or COS 7 cells), CV1 cells, quail QC1-3 cells, mouse L cells, mouse sarcoma cells, Bowes melanoma cells, or Chinese hamster ovary (CHO) cells, preferably CHO cells (e.g., CHO-K1 cells). A host cell according to the invention may be a directly engineered cell, e.g., a cell having undergone direct transfection with a vector or polynucleotide as disclosed herein, or it may be a daughter cell or progeny of the directly transfected cell.

When recombinant expression vectors encoding the antibody heavy chain and/or light chain variable domains as disclosed herein are introduced into host cells, the antibodies or antigen-binding fragments can be produced by culturing the host cells for a period of time sufficient to allow for expression of the antibody or antigen-binding fragment in the host cell or, preferably, to allow for secretion of the antibody or antigen-binding fragment into the culture medium in which the host cells are grown. Antibodies and/or antigen-binding fragments can be recovered from the culture medium using standard protein purification methods. Accordingly, the invention also provides a method for the production of an antibody (or an antigen-binding fragment) according to the invention, the method comprising culturing a host cell comprising a polynucleotide encoding the antibody or antigen-binding fragment or an expression vector containing said polynucleotide. The method may further comprise a step of isolating the expressed antibody or antigen-binding fragment from the culture (e.g., from the cell fraction and/or from the culture medium). The invention further provides an antibody or antigen-binding fragment obtainable by any of the methods described herein.

While it is possible to express the antibodies of the invention in either prokaryotic or eukaryotic host cells, expression of antibodies in eukaryotic cells is preferred, and expression in mammalian host cells is even more preferred, because such eukaryotic cells (and particularly mammalian cells) can be more readily used to assemble and secrete a properly folded and immunologically active antibody. Examples of mammalian host cells that can be used for expressing the antibodies or antigen-binding fragments of the present invention include, in particular, Chinese Hamster Ovary (CHO) cells, NS0 myeloma cells, COS cells, or SP2 cells. It is preferred that CHO cells (e.g., CHO-K1 cells) are used as host cells.

The transformed host cells can be grown in bioreactors and cultured according to techniques known in the art to achieve optimal cell growth. The antibody or antigen-binding fragment of the invention can then be isolated from the cell fraction or growth medium by conventional methods such as, e.g., affinity chromatography, gel filtration (size exclusion chromatography), anion exchange chromatography, cation exchange chromatography, hydrophobic interaction chromatography, high-pressure liquid chromatography (HPLC), reversed-phase HPLC, or immunoprecipitation.

Recombinantly produced antibodies or antigen-binding fragments may have variable regions and constant regions (if present) derived from the germline immunoglobulin sequences of the species from which they were isolated after standard immunization and selection procedures known in the art, e.g., may comprise the germline immunoglobulin sequences of a mouse, rat or rabbit. However, the antibody sequences can be subjected to in vitro mutagenesis wherein the CDR sequences are combined with framework sequences from another species, e.g., from a human, as is known in the process of humanization. Therefore, the amino acid sequences of the variable heavy-chain and light-chain domains can also be sequences that, while derived from and related to germline heavy-chain or light-chain sequences, may not naturally exist within any endogenous antibody germline repertoire.

Antibody derivatives can be produced, for example, by adding exogenous sequences to modify immunogenicity or reduce, enhance or modify binding, affinity, on-rate, off-rate, avidity, specificity, half-life, or any other suitable characteristic.

Also provided are humanized versions of the antibodies disclosed herein, particularly antibodies (and antigen-binding fragments thereof) comprising the CDRs disclosed herein above. Humanization refers to recombinantly engineering an antibody using CDRs derived from a non-human donor immunoglobulin, in the context of human-derived framework regions and constant domains. During the engineering, framework and/or CDR residues may be altered to preserve binding affinity and activity. Methods to humanize antibodies are well known in the art, as also described herein above. In particular, the present invention provides a human IgG antibody (e.g., a complete/intact human IgG1, IgG2, IgG3 or IgG4 antibody, particularly an IgG1 antibody) wherein the six CDRs are replaced by the six CDRs described herein above in any one of the options (A-1) to (A-25).

The present invention further provides a composition (preferably a pharmaceutical composition) comprising one or more pharmaceutically acceptable excipients and: (i) an antibody or an antigen-binding fragment thereof according to the invention; or (ii) a nucleic acid (or polynucleotide) encoding an antibody or antigen-binding fragment according to (i); or (iii) a vector comprising a polynucleotide according to (ii); or (iv) a host cell comprising a nucleic acid (or polynucleotide) according to (ii) and/or a vector according to (iii) that expresses an antibody or antigen-binding fragment according to (i).

The present invention furthermore relates to diagnostic applications of the antibodies and antigen-binding fragments provided herein. In particular, the antibodies and antigen-binding fragments according to the invention can be used as markers/reagents in immunohistochemistry, e.g., in order to diagnose cancer by testing a sample (e.g., a biopsy) from a subject and/or to stratify subjects/patients for cancer treatment and/or to assess treatment efficacy. Thus, the present invention relates to the in vitro use of an antibody or antigen-binding fragment (as described herein) in a diagnostic method, particularly in a method of diagnosing cancer in a subject using a sample (e.g., a blood sample or a biopsy sample, particularly a cancer/tumor biopsy sample) obtained from the subject. The invention likewise relates to an antibody or antigen-binding fragment (as described herein) for use in diagnosis, particularly for use in an in vivo method of diagnosing cancer in a subject, and relates to corresponding diagnostic methods.

As explained above, the present invention also relates to an antibody or antigen-binding fragment (as described herein) for use in the treatment of cancer. The invention further relates to the use of an antibody or antigen-binding fragment for the manufacture of a medicament for the treatment of cancer, as well as a method of treating cancer in a subject in need thereof, the method comprising administering to the subject a therapeutically effective amount of an antibody or antigen-binding fragment. The invention likewise relates to a corresponding treatment of cancer using a nucleic acid, a composition, a lipid particle, an antibody-drug conjugate, or an immune cell (particularly a T cell (e.g., an alpha-beta-T cell or a gamma-delta-T cell) or an NK cell) expressing a chimeric antigen receptor (CAR), as described herein.

The cancer to be treated in accordance with the present invention may be a solid cancer or a hematological cancer. Preferably, the cancer may be selected from lung cancer (e.g., small cell lung cancer or non-small cell lung cancer), renal cancer (or kidney cancer; e.g., renal carcinoma), gastrointestinal cancer, stomach cancer (or gastric cancer; e.g., gastric adenocarcinoma), colorectal cancer (e.g., colorectal carcinoma), colon cancer, anal cancer, genitourinary cancer, bladder cancer, urothelial cancer (e.g., urothelial carcinoma), liver cancer (e.g., hepatocellular carcinoma), pancreatic cancer (e.g., pancreatic adenocarcinoma or pancreatic ductal adenocarcinoma), ovarian cancer, cervical cancer, endometrial cancer, vaginal cancer, vulvar cancer, ovarian cancer (e.g., ovarian carcinoma), uterine cancer (e.g., uterine corpus cancer or uterine sarcoma), prostate cancer (e.g., hormone-refractory prostate cancer), testicular cancer, biliary tract cancer (or bile duct cancer; e.g., cholangiocarcinoma), hepatobiliary cancer, gallbladder cancer (e.g., gallbladder carcinoma), thyroid cancer (e.g., papillary thyroid cancer, follicular thyroid cancer, medullary thyroid cancer, or anaplastic thyroid cancer), neuroblastoma, brain cancer (e.g., glioblastoma), breast cancer (e.g., triple-negative breast cancer, including in particular COX-2 expressing triple-negative breast cancer, or breast cancer having a BRCA1 and/or BRCA2 gene mutation), head and/or neck cancer (e.g., head and neck squamous cell carcinoma), skin cancer, melanoma, Merkel-cell cancer (e.g., Merkel-cell carcinoma), epidermoid cancer, squamous cell cancer (or squamous cell carcinoma; including, e.g., oral squamous cell carcinoma/squamous-cell mouth carcinoma, squamous-cell skin cancer, squamous-cell lung carcinoma, squamous-cell thyroid carcinoma, squamous-cell esophageal carcinoma, or squamous-cell vaginal carcinoma), bone cancer (e.g., osteosarcoma or osteogenic sarcoma), fibrosarcoma, Ewing's sarcoma, Kaposi's sarcoma, malignant mesothelioma, esophageal cancer, laryngeal cancer, mouth cancer, thymic cancer (e.g., thymoma), neuroendocrine cancer (e.g., neuroendocrine carcinoma), goblet cell cancer (e.g., goblet cell carcinoid), spleen cancer, hematological cancer, leukemia (e.g., acute lymphoblastic leukemia, acute myeloid leukemia, chronic lymphocytic leukemia, or chronic myeloid leukemia), lymphoma (e.g., Hodgkin lymphoma or non-Hodgkin lymphoma, such as, e.g., follicular lymphoma or diffuse large B-cell lymphoma), and multiple myeloma. The cancer to be treated may further be a carcinoma or a sarcoma. Moreover, the cancer to be treated (including any one of the aforementioned specific types of cancer) may be a CCR8-positive cancer (particularly a cancer wherein CCR8 is expressed by the cancer cells, by cells of the tumor microenvironment, and/or by tumor-infiltrating cells (e.g., by tumor-infiltrating Treg cells). The cancer to be treated (including any one of the aforementioned specific types of cancer) may also be a metastatic cancer.

As explained above, the cancer to be treated in accordance with the present invention may be a hematological cancer (e.g., a lymphoma or a leukemia). In that case, the hematological cancer is preferably selected from: Hodgkin's lymphoma, including, e.g., nodular sclerosing subtype of Hodgkin's lymphoma, mixed-cellularity subtype of Hodgkin's lymphoma, lymphocyte-rich subtype of Hodgkin's lymphoma, or lymphocyte-depleted subtype of Hodgkin's lymphoma; non-Hodgkin's lymphoma, including, e.g., follicular non-Hodgkin's lymphoma, mantle cell lymphoma, or diffuse non-Hodgkin's lymphoma (e.g., diffuse large B-cell lymphoma or Burkitt's lymphoma); nodular lymphocyte predominant Hodgkin's lymphoma; peripheral/cutaneous T-cell lymphoma, including, e.g., mycosis fungoides, Sézary syndrome, T-zone lymphoma, lymphoepithelioid lymphoma (e.g., Lennert's lymphoma), or peripheral T-cell lymphoma; lymphosarcoma; a malignant immunoproliferative disorder, including, e.g., Waldenström's macroglobulinemia, alpha heavy chain disease, gamma heavy chain disease (e.g., Franklin's disease), or an immunoproliferative small intestinal disease (e.g., Mediterranean disease); multiple myeloma, including, e.g., Kahler's disease, or myelomatosis; plasma cell leukemia; lymphoid leukemia, including, e.g., acute lymphoblastic leukemia, chronic lymphocytic leukemia, subacute lymphocytic leukemia, prolymphocytic leukemia, hairy-cell leukemia (e.g., leukemic reticuloendotheliosis), or adult T-cell leukemia; myeloid leukemia, including, e.g., acute myeloid leukemia, chronic myeloid leukemia, subacute myeloid leukemia, myeloid sarcoma (e.g., chloroma, or granulocytic sarcoma), acute promyelocytic leukemia, or acute myelomonocytic leukemia; a myeloproliferative neoplastic disorder, including, e.g., polycythemia vera, essential thrombocythemia, or idiopathic myelofibrosis; monocytic leukemia; acute erythraemia or erythroleukemia, including, e.g., acute erythraemic myelosis, or Di Guglielmo's disease; chronic erythraemia, including, e.g., Heilmeyer-Schöner disease; acute megakaryoblastic leukemia; mast cell leukemia; acute panmyelosis; acute myelofibrosis; and Letterer-Siwe disease.

It is particularly preferred that the cancer to be treated in accordance with the present invention is selected from ovarian cancer, colorectal cancer, colon cancer, gastric cancer, esophageal cancer (e.g., esophageal squamous cell carcinoma, or esophageal adenocarcinoma), breast cancer (e.g., triple-negative breast cancer), lung cancer (e.g., non-small cell lung cancer), bladder cancer, uterine cancer (e.g., uterine corpus cancer), urothelial cancer (e.g., urothelial carcinoma), Kaposi's sarcoma, skin cancer (e.g., melanoma), head and/or neck cancer (e.g., head and neck squamous cell carcinoma), renal cancer (e.g., renal cell carcinoma), and lymphoma (e.g., non-Hodgkin lymphoma, such as cutaneous T-cell lymphoma (e.g., Sézary syndrome); even more preferably, the cancer is ovarian cancer, colorectal cancer, gastric cancer, breast cancer, or cutaneous T-cell lymphoma (e.g., Sézary syndrome).

The present invention furthermore relates to the use of an antibody or antigen-binding fragment provided herein as an antagonist (preferably an insurmountable antagonist) of the CCL1-CCR8 signaling pathway. The antibodies and antigen-binding fragments provided herein may be used as a research tool for antagonizing (preferably insurmountably antagonizing) the CCL1-CCR8 signaling pathway. Accordingly, the invention refers to the in vitro use of an antibody or antigen-binding fragment provided herein as an antagonist (preferably an insurmountable antagonist) of the CCL1-CCR8 signaling pathway, and particularly to the in vitro use of an antibody or antigen-binding fragment provided herein as a research tool agent acting as an antagonist (preferably an insurmountable antagonist) of the CCL1-CCR8 signaling pathway. The invention likewise relates to a method, particularly an in vitro method, of antagonizing (preferably insurmountably antagonizing) the CCL1-CCR8 signaling pathway, the method comprising the application of an antibody or antigen-binding fragment described herein. The invention further relates to a method of antagonizing (preferably insurmountably antagonizing) the CCL1-CCR8 signaling pathway, the method comprising applying an antibody or antigen-binding fragment provided herein to a test sample (e.g., a biological sample) or a test animal (i.e., a non-human test animal). The invention also provides a method of antagonizing (preferably insurmountably antagonizing) the CCL1-CCR8 signaling pathway, the method comprising contacting a test sample (e.g., a biological sample) or a test animal (i.e., a non-human test animal) with an antibody or antigen-binding fragment described herein. The terms “sample”, “test sample” and “biological sample” include, without being limited thereto: a cell, a cell culture or a cellular or subcellular extract; biopsied material obtained from an animal (e.g., a human), or an extract thereof. It is to be understood that the term “in vitro” is used in this specific context in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc. Moreover, it will be understood that the aforementioned in vitro uses and methods can also be characterized as “non-therapeutic” in vitro uses and methods.

The antibodies (or antigen-binding fragments) or the nucleic acids provided herein may be administered as such, or may be formulated as medicaments (or pharmaceutical compositions). The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

In particular, the pharmaceutical compositions may comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. In general, the pharmaceutical compositions can be formulated, e.g., as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The antibodies, the nucleic acids or the pharmaceutical compositions according to the present invention (referred to as “active agents” in the following) may be administered to a subject by any convenient route of administration, whether systemically/peripherally or at the site of desired action. In general, corresponding routes of administration include, in particular, oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

It is preferred that the antibodies, nucleic acids, or pharmaceutical compositions are administered parenterally, particularly by intravenous administration (e.g., by intravenous injection or intravenous infusion).

Examples of parenteral administration include one or more of: intravenously, intraarterially, intraperitoneally, intrathecally, intraventricularly, intraurethrally, intrasternally, intracardially, intracranially, intramuscularly or subcutaneously administering the active agents or pharmaceutical compositions, and/or by using infusion techniques.

For parenteral administration, the active agents can be used in the form of a sterile aqueous solution which may contain other substances, for example, enough salts or glucose to make the solution isotonic with blood. The aqueous solutions can be suitably buffered (preferably to a pH of from 3 to 9), if necessary. The preparation of suitable parenteral formulations under sterile conditions is readily accomplished by standard pharmaceutical techniques well known to those skilled in the art.

Said active agents or pharmaceutical compositions may also be administered orally, e.g., in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmelose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, this may be combined with various sweetening or flavoring agents, coloring matter or dyes, with emulsifying and/or suspending agents and with diluents such as water, ethanol, propylene glycol and glycerin, and combinations thereof.

For oral administration, the active agents or pharmaceutical compositions may be administered by oral ingestion, particularly by swallowing. The active agents or pharmaceutical compositions can thus be administered to pass through the mouth into the gastrointestinal tract, which can also be referred to as “oral-gastrointestinal” administration.

Alternatively, said active agents or pharmaceutical compositions can be administered in the form of a suppository or pessary, or may be applied topically in the form of a gel, hydrogel, lotion, solution, cream, ointment or dusting powder. The active agents of the present invention may also be dermally or transdermally administered, for example, by the use of a skin patch.

Said active agents or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped agents.

In particular, the present invention also relates to lipid particles (including, e.g., lipid nanoparticles or liposomes) containing one or more nucleic acids of the invention. The nucleic acids according to the invention may thus be provided in the form of a lipid particle. Such lipid particles and their preparation are known in the art and have been described, e.g., in: Müller R H et al., Eur J Pharm Biopharm, 2000, 50(1): 161-77, doi: 10.1016/s0939-6411(00) 00087-4; Saupe A et al., “Solid Lipid Nanoparticles”, in: Mozafari M R (ed.), “Nanocarrier Technologies”, 2006, Springer, doi: 10.1007/978-1-4020-5041-1_3; Cullis P R et al., Mol Ther, 2017, 25(7): 1467-75, doi: 10.1016/j.ymthe.2017.03.013; Hou X et al., Nat Rev Mater, 2021, 6(12): 1078-94, doi: 10.1038/s41578-021-00358-0; Tenchov R et al., ACS Nano, 2021, 15(11): 16982-17015, doi: 10.1021/acsnano.1c04996; or Sultana A et al., Medicine in Drug Discovery, 2022, 15:100134, doi: 10.1016/j.medidd.2022.100134; each of which is incorporated herein by reference.

The present invention thus relates to the active agents or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. A particularly preferred route of administration is parenteral administration (e.g., intravenous administration).

Typically, a physician will determine the actual dosage which will be most suitable for an individual subject. The specific dose level and frequency of dosage for any particular individual subject may be varied and will depend upon a variety of factors including the activity of the specific active agent employed, the metabolic stability and length of action of that agent, the age, body weight, general health, sex, diet, mode and time of administration, rate of excretion, drug combination, the severity of the particular condition, and the individual subject undergoing therapy.

The antibody (or antigen-binding fragment) or the nucleic acid according to the present invention, or a corresponding pharmaceutical composition, can be administered in monotherapy (e.g., without concomitantly administering any further therapeutic agents, or without concomitantly administering any further therapeutic agents against the same disease that is to be treated with the antibody or the nucleic acid). However, the antibody (or antigen-binding fragment) or the nucleic acid, or a corresponding pharmaceutical composition, can also be administered in combination with one or more further therapeutic agents. If the antibody or nucleic acid is used in combination with a second therapeutic agent active against the same disease or condition (e.g., cancer), the dose of each agent may differ from that when the corresponding agent is used alone, in particular, a lower dose of either one or both agents may be used. The combination of the antibody or nucleic acid with one or more further therapeutic agents may comprise the simultaneous/concomitant administration of the antibody or nucleic acid and the further therapeutic agent(s) (either in a single pharmaceutical formulation or in separate pharmaceutical formulations), or the sequential/separate administration of the antibody or nucleic acid and the further therapeutic agent(s). If administration is sequential, either the antibody or nucleic acid according to the invention or the one or more further therapeutic agents may be administered first. If administration is simultaneous, the one or more further therapeutic agents may be included in the same pharmaceutical formulation as the antibody or nucleic acid, or they may be administered in two or more different (separate) pharmaceutical formulations. The use of separate pharmaceutical formulations is particularly advantageous if the antibody or nucleic acid and the further therapeutic agent(s) are administered via different routes of administration, e.g., if the antibody or nucleic acid is administered parenterally (e.g., intravenously) while the further therapeutic agent(s) is/are administered orally.

Preferably, the one or more further therapeutic agents to be administered in combination with an antibody (or antigen-binding fragment) or a nucleic acid according to the present invention are one or more anticancer drugs. The anticancer drug(s) to be administered in combination with an antibody or nucleic acid according to the invention may be selected, e.g., from: a tumor angiogenesis inhibitor (e.g., a protease inhibitor, an epidermal growth factor receptor kinase inhibitor, or a vascular endothelial growth factor receptor kinase inhibitor); a cytotoxic drug (e.g., an antimetabolite, such as purine and pyrimidine analog antimetabolites); an antimitotic agent (e.g., a microtubule stabilizing drug or an antimitotic alkaloid); a platinum coordination complex; an anti-tumor antibiotic; an alkylating agent (e.g., a nitrogen mustard or a nitrosourea); an endocrine agent (e.g., an adrenocorticosteroid, an androgen, an anti-androgen, an estrogen, an anti-estrogen, an aromatase inhibitor, a gonadotropin-releasing hormone agonist, or a somatostatin analog); or a compound that targets an enzyme or receptor that is overexpressed and/or otherwise involved in a specific metabolic pathway that is deregulated (or misregulated) in the tumor cell (e.g., ATP and GTP phosphodiesterase inhibitors, histone deacetylase inhibitors, protein kinase inhibitors (such as serine, threonine and tyrosine kinase inhibitors, e.g., Abelson protein tyrosine kinase inhibitors) and the various growth factors, their receptors and corresponding kinase inhibitors (such as epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors)); methionine, aminopeptidase inhibitors, proteasome inhibitors, cyclooxygenase inhibitors (e.g., cyclooxygenase-1 or cyclooxygenase-2 inhibitors), topoisomerase inhibitors (e.g., topoisomerase I inhibitors or topoisomerase II inhibitors), poly ADP ribose polymerase inhibitors (PARP inhibitors), epidermal growth factor receptor (EGFR) inhibitors/antagonists, targeted cancer therapies, cellular cancer therapies, and cancer vaccines.

An alkylating agent which can be used as an anticancer drug in combination with an antibody or nucleic acid of the present invention may be, for example, a nitrogen mustard (such as cyclophosphamide, mechlorethamine (chlormethine), uramustine, melphalan, chlorambucil, ifosfamide, bendamustine, or trofosfamide), a nitrosourea (such as carmustine, streptozocin, fotemustine, lomustine, nimustine, prednimustine, ranimustine, or semustine), an alkyl sulfonate (such as busulfan, mannosulfan, or treosulfan), an aziridine (such as hexamethylmelamine (altretamine), triethylenemelamine, ThioTEPA (N,N′N′-triethylenethiophosphoramide), carboquone, or triaziquone), a hydrazine (such as procarbazine), a triazene (such as dacarbazine), or an imidazotetrazine (such as temozolomide). A platinum coordination complex which can be used as an anticancer drug in combination with an antibody or nucleic acid of the present invention may be, for example, cisplatin, carboplatin, nedaplatin, oxaliplatin, satraplatin, or triplatin tetranitrate. A cytotoxic drug which can be used as an anticancer drug in combination with an antibody or nucleic acid of the present invention may be, for example, an antimetabolite, including folic acid analogue antimetabolites (such as aminopterin, methotrexate, pemetrexed, or raltitrexed), purine analogue antimetabolites (such as cladribine, clofarabine, fludarabine, 6-mercaptopurine (including its prodrug form azathioprine), pentostatin, or 6-thioguanine), and pyrimidine analogue antimetabolites (such as cytarabine, decitabine, 5-fluorouracil (including its prodrug forms capecitabine and tegafur), floxuridine, gemcitabine, enocitabine, or sapacitabine). An antimitotic agent which can be used as an anticancer drug in combination with an antibody or nucleic acid of the present invention may be, for example, a taxane (such as docetaxel, larotaxel, ortataxel, paclitaxel/taxol, tesetaxel, or nab-paclitaxel (e.g., Abraxane®)), a Vinca alkaloid (such as vinblastine, vincristine, vinflunine, vindesine, or vinorelbine), an epothilone (such as epothilone A, epothilone B, epothilone C, epothilone D, epothilone E, or epothilone F) or an epothilone B analogue (such as ixabepilone/azaepothilone B). An anti-tumor antibiotic which can be used as an anticancer drug in combination with an antibody or nucleic acid of the present invention may be, for example, an anthracycline (such as aclarubicin, daunorubicin, doxorubicin, epirubicin, idarubicin, amrubicin, pirarubicin, valrubicin, or zorubicin), an anthracenedione (such as mitoxantrone, or pixantrone) or an anti-tumor antibiotic isolated from Streptomyces (such as actinomycin (including actinomycin D), bleomycin, mitomycin (including mitomycin C), or plicamycin). A tyrosine kinase inhibitor which can be used as an anticancer drug in combination with an antibody or nucleic acid of the present invention may be, for example, axitinib, bosutinib, cediranib, dasatinib, erlotinib, gefitinib, imatinib, lapatinib, lestaurtinib, nilotinib, semaxanib, sorafenib, sunitinib, axitinib, nintedanib, ponatinib, vandetanib, or vemurafenib. A topoisomerase inhibitor which can be used as an anticancer drug in combination with an antibody or nucleic acid of the present invention may be, for example, a topoisomerase I inhibitor (such as irinotecan, topotecan, camptothecin, belotecan, rubitecan, or lamellarin D) or a topoisomerase II inhibitor (such as amsacrine, etoposide, etoposide phosphate, teniposide, or doxorubicin). A PARP inhibitor which can be used as an anticancer drug in combination with an antibody or nucleic acid of the present invention may be, for example, niraparib, olaparib, rucaparib, talazoparib, veliparib, pamiparib (BGB-290), BMN-673, CEP 9722, MK 4827, E7016, or 3-aminobenzamide. An EGFR inhibitor/antagonist which can be used as an anticancer drug in combination with an antibody or nucleic acid of the present invention may be, for example, gefitinib, erlotinib, lapatinib, afatinib, neratinib, osimertinib, brigatinib, dacomitinib, vandetanib, pelitinib, canertinib, icotinib, poziotinib, ABT-414, AV-412, PD 153035, PKI-166, BMS-690514, CUDC-101, AP26113, XL647, cetuximab, panitumumab, zalutumumab, nimotuzumab, or matuzumab. Further anticancer drugs may also be used in combination with an antibody or nucleic acid of the present invention. The anticancer drugs may comprise biological or chemical molecules, like TNF-related apoptosis-inducing ligand (TRAIL), tamoxifen, amsacrine, bexarotene, estramustine, irofulven, trabectedin, cetuximab, panitumumab, tositumomab, alemtuzumab, bevacizumab, edrecolomab, gemtuzumab, alvocidib, seliciclib, aminolevulinic acid, methyl aminolevulinate, efaproxiral, porfimer sodium, talaporfin, temoporfin, verteporfin, alitretinoin, tretinoin, anagrelide, arsenic trioxide, atrasentan, bortezomib, carmofur, celecoxib, demecolcine, elesclomol, elsamitrucin, etoglucid, lonidamine, lucanthone, masoprocol, mitobronitol, mitoguazone, mitotane, oblimersen, omacetaxine, sitimagene, ceradenovec, tegafur, testolactone, tiazofurine, tipifarnib, vorinostat, iniparib, or copanlisib.

Also biological drugs, like antibodies or antibody fragments/constructs directed against cancer or tumor markers/factors/cytokines involved in proliferative diseases can be employed in cotherapy approaches with the an antibody or nucleic acid of the invention. Examples of such biological molecules are anti-HER2 antibodies (e.g. trastuzumab, Herceptin®), anti-CD20 antibodies (e.g. Rituximab, Rituxan®, MabThera®, Reditux®), anti-CD19/CD3 constructs, or anti-TNF antibodies (see, e.g., Taylor P C, Curr Opin Pharmacol, 2003, 3(3): 323-328). Further antibodies and/or antibody fragments/constructs to be used in cotherapy approaches with any of the antibodies or nucleic acids of the invention can be found, e.g., in: Taylor P C, Curr Opin Pharmacol, 2003, 3(3): 323-328; or Roxana A, Maedica, 2006, 1(1): 63-65.

An anticancer drug which can be used in combination with an antibody or nucleic acid of the present invention may also be an immune-oncology therapeutic (such as an antibody or an antibody fragment/construct) targeting, e.g., any one of CTLA-4, PD-1, PD-L1, TIGIT, TIM3, LAG3, OX40, ICOS, CSF1R, IDO, CD40, CD25, or CCR4. Such immune-oncology therapeutics include, e.g., an anti-CTLA-4 antibody (particularly an antagonistic or pathway-blocking anti-CTLA-4 antibody; e.g., ipilimumab or tremelimumab), an anti-PD-1 antibody (particularly an antagonistic or pathway-blocking anti-PD-1 antibody; e.g., nivolumab (BMS-936558), pembrolizumab (MK-3475), pidilizumab (CT-011), cemiplimab, dostarlimab, spartalizumab, camrelizumab, sintilimab, tislelizumab, toripalimab, zimberelimab, AMP-224, AMP-514 (or MEDI0680), JTX-4014, INCMGA00012 (or MGA012), or APE02058), an anti-PD-L1 antibody (particularly a pathway-blocking anti-PD-L1 antibody; e.g., atezolizumab, avelumab, durvalumab, KN035, CK-301, BMS-936559, MEDI4736, MPDL3280A (RG7446), MDX-1105, MEDI6469, or bintrafusp alfa), an anti-TIGIT antibody (e.g., tiragolumab, vibostolimab, domvanalimab, etigilimab, BMS-986207, EOS-448, COM902, ASP8374, SEA-TGT, BGB-A1217, IBI939, or M6223), an anti-TIM3 antibody (particularly a pathway-blocking anti-TIM3 antibody; e.g., cobolimab (or TSR-022), sabatolimab (or MBG453), BMS-986258, Sym023, TQB2618, LY3321367, or SHR-1702), an anti-LAG3 antibody (particularly an antagonistic or pathway-blocking anti-LAG3 antibody; e.g., relatlimab (or BMS-986016), ieramilimab, encelimab, tebotelimab, REGN3767, FS118, IMP701, or IMP731), an anti-OX40 antibody (particularly an agonistic anti-OX40 antibody; e.g., ivuxolimab (or PF-8600), MEDI0562, MEDI6383, MEDI6469, INCAGNO1949, ABBV-368, BAT6026, BGB-A445, YH-002, BMS 986178, INBRX-106, IBI101, or MOXR0916), an anti-ICOS antibody (particularly an agonistic anti-ICOS antibody; e.g., feladilimab (or GSK3359609), vopratelimab (or JTX-2011), BMS-986226, alomfilimab (or SAR445256 or KY1044), MEDI-570, or XmAb23104), an anti-CSF1R antibody (particularly a pathway-blocking anti-CSF1R antibody; e.g., IMC-CS4 or RG7155), an anti-IDO antibody (particularly a pathway-blocking anti-IDO antibody), an anti-CD40 antibody (particularly an agonistic anti-CD40 antibody; e.g., CP-870,893 or Chi Lob 7/4), an anti-CD25 antibody (particularly a Treg-depleting anti-CD25 antibody; e.g., daclizumab, inolimomab, or RG6292), or an anti-CCR4 antibody (particularly a Treg-depleting anti-CCR4 antibody; e.g., mogamulizumab). Further examples of immuno-oncology therapeutics include any of anti-HER2 antibodies (e.g., trastuzumab), anti-CD20 antibodies (e.g., rituximab), anti-CD19/CD3 constructs, and anti-TNF antibodies.

In particular, it is preferred that an antibody or nucleic acid of the present invention, or a corresponding pharmaceutical composition or a lipid particle, is administered in combination with an immune checkpoint inhibitor, more preferably an antibody (or an antigen-binding fragment thereof) directed against any of CTLA-4, PD-1, PD-L1, TIGIT, TIM3, LAG3, OX40, or ICOS. Corresponding preferred examples include, but are not limited to, any one of the anti-CTLA-4 antibodies ipilimumab or tremelimumab, any one of the anti-PD-1 antibodies nivolumab, pembrolizumab, pidilizumab, cemiplimab, dostarlimab, spartalizumab, camrelizumab, sintilimab, tislelizumab, toripalimab, zimberelimab, AMP-224, AMP-514, JTX-4014, INCMGA00012, or APE02058, any one of the anti-PD-L1 antibodies atezolizumab, avelumab, durvalumab, KN035, CK-301, BMS-936559, MEDI4736, MPDL3280A, MDX-1105, MEDI6469 or bintrafusp alfa, any one of the anti-TIGIT antibodies tiragolumab, vibostolimab, domvanalimab, etigilimab, BMS-986207, EOS-448, COM902, ASP8374, SEA-TGT, BGB-A1217, IBI939 or M6223, any one of the anti-TIM3 antibodies cobolimab, sabatolimab, BMS-986258, Sym023, TQB2618, LY3321367, or SHR-1702, any one of the anti-LAG3 antibodies relatlimab, ieramilimab, encelimab, tebotelimab, REGN3767, FS118, IMP701, or IMP731, any one of the anti-OX40 antibodies ivuxolimab, MEDI0562, MEDI6383, MEDI6469, INCAGNO1949, ABBV-368, BAT6026, BGB-A445, YH-002, BMS 986178, INBRX-106, IBI101, or MOXR0916, and/or any one of the anti-ICOS antibodies alomfilimab, feladilimab, vopratelimab, BMS-986226, MEDI-570, or XmAb23104. The present invention thus relates to an antibody or nucleic acid (as described herein), or a pharmaceutical composition or a lipid particle, for use in the treatment of cancer, wherein the antibody or the nucleic acid (or the pharmaceutical composition or the lipid particle) is to be administered in combination with one or more immune checkpoint inhibitors, wherein said one or more immune checkpoint inhibitors are preferably selected from anti-CTLA-4 antibodies, anti-PD-1 antibodies, anti-PD-L1 antibodies, anti-TIGIT antibodies, anti-TIM3 antibodies, anti-LAG3 antibodies, anti-OX40 antibodies and/or anti-ICOS antibodies; more preferably, said one or more immune checkpoint inhibitors are selected from ipilimumab, tremelimumab, nivolumab, pembrolizumab, pidilizumab, cemiplimab, dostarlimab, spartalizumab, camrelizumab, sintilimab, tislelizumab, toripalimab, zimberelimab, AMP-224, AMP-514, JTX-4014, INCMGA00012, APE02058, atezolizumab, avelumab, durvalumab, KN035, CK-301, BMS-936559, MEDI4736, MPDL3280A, MDX-1105, MEDI6469, bintrafusp alfa, tiragolumab, vibostolimab, domvanalimab, etigilimab, BMS-986207, EOS-448, COM902, ASP8374, SEA-TGT, BGB-A1217, IBI939, M6223, cobolimab, sabatolimab, BMS-986258, Sym023, TQB2618, LY3321367, SHR-1702, relatlimab, ieramilimab, encelimab, tebotelimab, REGN3767, FS118, IMP701, IMP731, ivuxolimab, MEDI0562, MEDI6383, MEDI6469, INCAGN01949, ABBV-368, BAT6026, BGB-A445, YH-002, BMS 986178, INBRX-106, IBI101, MOXR0916, alomfilimab, feladilimab, vopratelimab, BMS-986226, MEDI-570, and XmAb23104.

Accordingly, in preferred embodiments, the present invention relates to the antibody (or antigen-binding fragment) for use in the treatment of cancer, wherein said treatment comprises the combined administration of one or more immune checkpoint inhibitors. The invention also relates to any of the nucleic acids, compositions or lipid particles described herein for use in the treatment of cancer, wherein said treatment comprises the combined administration of one or more immune checkpoint inhibitors. The invention further relates to the use of an antibody or antigen-binding fragment according to the invention (or a nucleic acid, a composition, or a lipid particle according to the invention) for the manufacture of a medicament for the treatment of cancer, wherein said treatment comprises the combined administration of one or more immune checkpoint inhibitors. The invention likewise relates to a method of treating cancer in a subject in need thereof, the method comprising administering to the subject a therapeutically effective amount of an antibody or antigen-binding fragment according to the invention (or a nucleic acid, a composition, or a lipid particle according to the invention) in combination with a therapeutically effective amount of one or more immune checkpoint inhibitors. Said one or more immune checkpoint inhibitors are preferably selected from anti-CTLA-4 antibodies, anti-PD-1 antibodies, anti-PD-L1 antibodies, anti-TIGIT antibodies, anti-TIM3 antibodies, anti-LAG3 antibodies, anti-OX40 antibodies, and anti-ICOS antibodies (or any combination thereof). More preferably said one or more immune checkpoint inhibitors are selected from ipilimumab, tremelimumab, nivolumab, pembrolizumab, pidilizumab, cemiplimab, dostarlimab, spartalizumab, camrelizumab, sintilimab, tislelizumab, toripalimab, zimberelimab, AMP-224, AMP-514, JTX-4014, INCMGA00012, APE02058, atezolizumab, avelumab, durvalumab, KN035, CK-301, BMS-936559, MEDI4736, MPDL3280A, MDX-1105, MEDI6469, bintrafusp alfa, tiragolumab, vibostolimab, domvanalimab, etigilimab, BMS-986207, EOS-448, COM902, ASP8374, SEA-TGT, BGB-A1217, IBI 939, M6223, cobolimab, sabatolimab, BMS-986258, Sym023, TQB2618, LY3321367, SHR-1702, relatlimab, ieramilimab, encelimab, tebotelimab, REGN3767, FS118, IMP701, IMP731, ivuxolimab, MEDI0562, MEDI6383, MEDI6469, INCAGNO1949, ABBV-368, BAT6026, BGB-A445, YH-002, BMS 986178, INBRX-106, IBI101, MOXR0916, alomfilimab, feladilimab, vopratelimab, BMS-986226, MEDI-570, and XmAb23104.

As explained above, for the treatment of cancer, the antibody or nucleic acid according to the present invention (or a corresponding pharmaceutical composition or a lipid particle) may also be used in combination with targeted cancer therapy, e.g., by administering, in combination with said antibody or nucleic acid, one or more therapeutic agents targeting genes or proteins that support the survival and/or growth of cancer cells, preferably one or more tyrosine kinase inhibitors (TKI). The tyrosine kinase inhibitors to be administered in combination with the antibody or nucleic acid according to the present invention are not particularly limited, but are preferably one or more agents selected from anaplastic lymphoma kinase (ALK) inhibitors (such as, e.g., alectinib, alkotinib, belizatinib, brigatinib, ceritinib, crizotinib, ensartinib, entrectinib, foritinib, lorlatinib, repotrectinib, CEP-37440, TQ-B3139, PLB1003, or TPX-0131), Bcr-Abl tyrosine-kinase inhibitors (such as, e.g., asciminib, bafetinib, bosutinib, dasatinib, imatinib, nilotinib, ponatinib, or rebastinib), EGFR inhibitors/antagonists (such as, e.g., afatinib, brigatinib, canertinib, dacomitinib, erlotinib, gefitinib, icotinib, lapatinib, neratinib, osimertinib, pelitinib, vandetanib, ABT-414, AP26113, AV-412, BMS-690514, CUDC-101, PD 153035, PKI-166, XL647, cetuximab, matuzumab, nimotuzumab, panitumumab, or zalutumumab), FMS-like tyrosine kinase 3 (FLT3) inhibitors (such as, e.g., gilteritinib, lestaurtinib, midostaurin, quizartinib, sorafenib, or sunitinib), fibroblast growth factor receptor (FGFR) inhibitors/antagonists (such as, e.g., erdafitinib, BGJ398, PD173074, or ZD4547), Janus kinase (JAK) inhibitors (such as, e.g., abrocitinib, baricitinib, cerdulatinib, delgocitinib, deucravacitinib, fedratinib, filgotinib, gandotinib, lestaurtinib, momelotinib, oclacitinib, pacritinib, peficitinib, ruxolitinib, tofacitinib (or tasocitinib), upadacitinib, CHZ868, or cucurbitacin I (or JSI-124)), neurotrophic tyrosine receptor kinase (NTRK) inhibitors (such as, e.g., entrectinib, larotrectinib, repotrectinib, or selitrectinib), vascular endothelial growth factor receptor (VEGFR) inhibitors/antagonists (such as, e.g., apatinib (or rivoceranib), axitinib, bevacizumab, cabozantinib, cediranib, intedanib, itraconazole, lenvatinib, lucitanib, motesanib, nintedanib, pazopanib, regorafenib, semaxanib, sorafenib, sunitinib, vandetanib, or vatalanib), BRAF inhibitors (such as, e.g., dabrafenib, encorafenib, sorafenib, vemurafenib, GDC-0879, or PLX-4720), Bruton's tyrosine kinase (BTK) inhibitors (such as, e.g., acalabrutinib, evobrutinib, fenebrutinib, ibrutinib, pirtobrutinib, spebrutinib, tirabrutinib, tolebrutinib, zanubrutinib, ABBV-105, GS-4059 (or ONO-4059), or HM71224), mitogen-activated protein kinase kinase (MEK) inhibitors (such as, e.g., binimetinib, cobimetinib, selumetinib, trametinib, CI-1040, PD035901, PD-325901, or TAK-733), and/or cyclin-dependent-kinase 4/6 (CDK4/6) inhibitors (such as, e.g., abemaciclib, palbociclib, ribociclib, trilaciclib, voruciclib, or zotiraciclib).

The antibody or nucleic acid according to the present invention (or a corresponding pharmaceutical composition or a lipid particle) may also be used, for the treatment of cancer, in combination with cellular cancer therapy, e.g., in combination with chimeric antigen receptor (CAR) cell-based therapy, particularly CAR T cell therapy (e.g., using CAR alpha-beta-T cells or CAR gamma-delta-T cells) or CAR NK cell therapy. Corresponding examples include, in particular, axicabtagene ciloleucel, brexucabtagene autoleucel, ciltacabtegene autoleucel, idecabtagene vicleucel, lisocabtagene maraleucel, or tisagenlecleucel.

Moreover, one or more cancer vaccines may also be used in combination with an antibody or nucleic acid of the present invention (or a corresponding pharmaceutical composition or a lipid particle) for the treatment of cancer. Such cancer vaccines may be, e.g., cell-based cancer vaccines, protein- or peptide-based cancer vaccines, or nucleic acid-based cancer vaccines. Corresponding examples include, in particular, AdV-tk, Bacillus Calmette-Guérin (BCG) vaccine, BiovaxID, DC-adenovirus p53 vaccine, GI-6301, GVAX, NeoVax, NeuVax, oncophage (or HSPPC-96), sipuleucel-T, or talimogene laherparepvec.

The combinations referred to above may conveniently be presented for use in the form of a pharmaceutical formulation. The individual components of such combinations may be administered either sequentially or simultaneously/concomitantly in separate or combined pharmaceutical formulations by any convenient route. When administration is sequential, either the antibody or nucleic acid of the present invention (or the corresponding composition or lipid particle) or the further therapeutic agent(s) may be administered first. When administration is simultaneous, the combination may be administered either in the same pharmaceutical composition or in different pharmaceutical compositions. When combined in the same formulation, it will be appreciated that the two or more active agents must be stable and compatible with each other and the other components of the formulation. When formulated separately, they may be provided in any convenient formulation and may be administered by any convenient route.

The antibody or nucleic acid of the present invention can also be administered in combination with physical therapy, such as radiotherapy. Radiotherapy may commence before, after, or simultaneously with administration of the antibody or nucleic acid. For example, radiotherapy may commence about 1 to 10 minutes, about 1 to 10 hours, or about 24 to 72 hours after administration of the antibody or the nucleic acid. The subject/patient is exposed to radiation, preferably gamma radiation, whereby the radiation may be provided in a single dose or in multiple doses that are administered over several hours, days and/or weeks. Gamma radiation may be delivered according to standard radiotherapeutic protocols using standard dosages and regimens.

The present invention thus relates to an antibody or a nucleic acid (as provided herein), or a corresponding pharmaceutical composition or a lipid particle, for use in the treatment of cancer, wherein the antibody or the nucleic acid (or the pharmaceutical composition or the lipid particle) is to be administered in combination with one or more anticancer drugs (including any one or more of the specific anticancer drugs described herein above) and/or in combination with radiotherapy.

Yet, the antibody or the nucleic acid according to the invention can also be used in monotherapy, particularly in the monotherapeutic treatment of cancer (i.e., without administering any other anticancer agents until the treatment with the antibody or the nucleic acid is terminated). Accordingly, the invention also relates to an antibody or a nucleic acid (as described herein) for use in the monotherapeutic treatment of cancer.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

As used herein, the term “treatment” (or “treating”) in relation to a disease or disorder (e.g., cancer) refers to the management and care of a patient for the purpose of combating the disease or disorder, such as to reverse, alleviate, inhibit or delay the disease or disorder, or one or more symptoms of such disease or disorder. It also refers to the administration of a substance (e.g., an antibody or a nucleic acid) or a composition for the purpose of preventing the onset of symptoms of the disease or disorder, alleviating such symptoms, or eliminating the disease or disorder. The “treatment” may thus be, e.g., curative, ameliorating, palliative, or prophylactic. However, the present invention also specifically relates to the non-prophylactic treatment of any disease/disorder described herein, including in particular the non-prophylactic treatment of cancer. It is thus preferred that the “treatment” is curative, ameliorating or palliative.

As used herein, the terms “optional”, “optionally” and “may” denote that the indicated feature may be present but can also be absent. Whenever the term “optional”, “optionally” or “may” is used, the present invention specifically and individually relates to both possibilities, i.e., that the corresponding feature is present or, alternatively, that the corresponding feature is absent. For example, if a component of a composition is indicated to be “optional”, the invention specifically relates to both possibilities, i.e., that the corresponding component is present (contained in the composition) or that the corresponding component is absent from the composition.

As used herein, unless explicitly indicated otherwise or contradicted by context, the terms “a”, “an” and “the” are used interchangeably with “one or more” and “at least one”. Thus, for example, a composition comprising “an” excipient can be interpreted as referring to a composition comprising “one or more” excipients.

As used herein, the term “amino acid” refers, in particular, to any one of the 20 standard proteinogenic α-amino acids (i.e., Ala, Arg, Asn, Asp, Cys, Glu, Gln, Gly, His, Ile, Leu, Lys, Met, Phe, Pro, Ser, Thr, Trp, Tyr, and Val) but also to non-proteinogenic and/or non-standard α-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., γ-aminobutyric acid, isoglutamine, or statine) and/or δ-amino acids as well as any other compound comprising at least one carboxylic acid group and at least one amino group. Unless defined otherwise, an “amino acid” preferably refers to an α-amino acid, more preferably to any one of the 20 standard proteinogenic α-amino acids (which can be present as the L-isomer or the D-isomer, and are preferably present as the L-isomer).

The terms “peptide”, “polypeptide” and “protein” are used herein interchangeably and refer to a polymer of two or more amino acids linked via amide bonds that are formed between an amino group of one amino acid and a carboxyl group of another amino acid. The amino acids comprised in the peptide or protein, which are also referred to as amino acid residues, may be selected from the 20 standard proteinogenic-amino acids (i.e., Ala, Arg, Asn, Asp, Cys, Glu, Gln, Gly, His, Ile, Leu, Lys, Met, Phe, Pro, Ser, Thr, Trp, Tyr, and Val) but also from non-proteinogenic and/or non-standard α-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., γ-aminobutyric acid or isoglutamine) and/or ò-amino acids. Preferably, the amino acid residues comprised in the peptide or protein are selected from α-amino acids, more preferably from the 20 standard proteinogenic α-amino acids (which can be present as the L-isomer or the D-isomer, and are preferably all present as the L-isomer). The peptide or protein may be unmodified or may be modified, e.g., at its N-terminus, at its C-terminus and/or at a functional group in the side chain of any of its amino acid residues (particularly at the side chain functional group of one or more Lys, His, Ser, Thr, Tyr, Cys, Asp, Glu, and/or Arg residues). Such modifications may include, e.g., post-translational modifications and/or the attachment of any of the protecting groups described for the corresponding functional groups in: Wuts P G & Greene T W, Greene's protective groups in organic synthesis, John Wiley & Sons, 2006. Such modifications may also include the covalent attachment of one or more polyethylene glycol (PEG) chains (forming a PEGylated peptide or protein), the glycosylation and/or the acylation with one or more fatty acids (e.g., one or more C8-30 alkanoic or alkenoic acids; forming a fatty acid acylated peptide or protein). Moreover, such modified peptides or proteins may also include peptidomimetics, provided that they contain at least two amino acids that are linked via an amide bond (formed between an amino group of one amino acid and a carboxyl group of another amino acid). The amino acid residues comprised in the peptide or protein may, e.g., be present as a linear molecular chain (forming a linear peptide or protein) or may form one or more rings (corresponding to a cyclic peptide or protein). The peptide or protein may also form oligomers consisting of two or more identical or different molecules.

All amino acid sequences provided herein are presented in N→C direction, i.e., starting with the N-terminus (the most N-terminal amino acid residue) and ending with the C-terminus (the most C-terminal amino acid residue).

Moreover, unless explicitly indicated otherwise or contradicted by context, any reference to a sequence (or partial sequence) from a defined nucleotide sequence or a defined amino acid sequence (e.g., defined by its SEQ ID) is to be understood as relating to a continuous sequence (or a continuous partial sequence) from the corresponding defined sequence. Thus, for example, any reference to a partial sequence of X to Y amino acid residues from a larger amino acid sequence defined by its SEQ ID is to be understood as relating to a continuous (uninterrupted) sequence of X to Y amino acid residues from the defined larger amino acid sequence.

The term “% sequence identity”, as used herein, e.g., in connection with amino acid sequences of proteins/peptides and/or in connection with nucleotide sequences of nucleic acid molecules, describes the number of matches of identical amino acid residues (or nucleotides) of two or more aligned sequences as compared to the number of amino acid residues (or nucleotides) making up the overall length of the compared sequences (or the overall compared portions thereof). Using an alignment of two or more sequences or subsequences, the percentage of amino acid residues (or nucleotides) that are the same may be determined when the (sub) sequences are compared and aligned for maximum correspondence over a window of comparison, or over a designated region as measured using a sequence comparison algorithm as known in the art, or when manually aligned and visually inspected. Examples of algorithms that can be used for determining sequence identity include, in particular, those based on the NCBI BLAST algorithm (Altschul S F et al., Nucleic Acids Res, 1997, 25(17): 3389-402, doi: 10.1093/nar/25.17.3389), CLUSTALW (Thompson J D et al., Nucleic Acids Res, 1994, 22(22): 4673-80, doi: 10.1093/nar/22.22.4673), or FASTA (Pearson W R et al., Proc Natl Acad Sci USA, 1988, 85(8): 2444-8, doi: 10.1073/pnas.85.8.2444). Although the FASTA algorithm typically does not consider internal non-matching deletions or additions in sequences, i.e. gaps, in its calculation, this can be corrected manually to avoid an overestimation of the % sequence identity. CLUSTALW, however, does take sequence gaps into account in its identity calculations.

Furthermore, it is to be understood that wherever a numerical range is provided/described herein, all values and subranges encompassed by the respective numerical range are specifically provided by the invention. Accordingly, the present invention specifically and individually relates to each value that falls within a numerical range described herein, as well as each and any subrange encompassed by a numerical range described herein.

As used herein, the term “about” preferably refers to +10% of the indicated numerical value, more preferably to +5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint-5% to the upper endpoint +5%, and even more preferably to the range defined by the exact numerical values of the lower endpoint and the upper endpoint. If the term “about” is used in connection with the endpoint of an open-ended range, it preferably refers to the corresponding range starting from the lower endpoint-10% or from the upper endpoint+10%, more preferably to the range starting from the lower endpoint-5% or from the upper endpoint+5%, and even more preferably to the open-ended range defined by the exact numerical value of the corresponding endpoint. If the term “about” is used in connection with a parameter that is quantified in integers, such as the number of nucleotides in a given nucleic acid or the number of amino acid residues in a given peptide or protein, the numbers corresponding to +10% or +5% of the indicated numerical value are to be rounded to the nearest integer (using the tie-breaking rule “round half up”). Thus, for example, the expression “about 25 amino acid residues” preferably refers to the range of 23 to 28 amino acid residues, more preferably to the range of 24 to 26 amino acid residues, and even more preferably refers to the specific value of 25 amino acid residues.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes (i.e., also provides a specific disclosure of) the meaning of “A consisting essentially of B and C” and the meaning of “A consisting of B and C” (i.e., no other components than B and C are comprised in A).

As used herein, the term “having” (or “has” or “have”), particularly when used in connection with an amino acid sequence or a nucleotide sequence, means “comprising”. In addition, the term “having” also includes the preferred meaning “consisting of”. Accordingly, an expression such as, e.g., “a VH domain having the sequence . . . ” indicates that the VH domain “comprises” the indicated sequence, and that it preferably “consists of” the indicated sequence.

Unless specifically indicated otherwise or contradicted by context, all properties and parameters (including functional parameters) referred to herein are preferably to be determined (and, likewise, all assays and tests referred to herein are preferably to be conducted) at standard conditions, particularly at standard ambient temperature and pressure conditions, e.g., at a temperature of 25° C. (298.15 K) and at an absolute pressure of 101.325 kPa (1 atm). Assays determining a biological activity (e.g., an enzymatic activity) can also be conducted at a temperature of 37° C.

Moreover, unless indicated otherwise, any reference to a database, an industry standard, a pharmacopeia, or a manufacturer's manual refers to the corresponding latest version that was available either at the filing date or at the earliest priority date (which may also be referred to as the earliest filing date) of the present specification, preferably the latest version that was available at the earliest priority date of the present specification.

All amino acid sequences and all nucleotide sequences referred to herein are also described in a concurrently filed sequence listing, which is incorporated herein by reference in its entirety. In the case of conflict between any sequence specified in the present disclosure and the corresponding sequence specified in the accompanying sequence listing, the present invention specifically and individually relates to each one of the respective sequences.

It is to be understood that the present invention specifically and individually relates to each and every combination of features and embodiments described herein, including any combination of general and/or preferred features/embodiments. Thus, in accordance with the present invention, each such combination is specifically envisaged herein.

In this specification, a number of documents including patent applications, scientific literature and manufacturers' manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The present invention particularly relates to the following items:
1. A monoclonal antibody or an antigen-binding fragment thereof, wherein said antibody or antigen-binding fragment specifically binds to human CCR8 and is an insurmountable antagonist of the CCL1-CCR8 signaling pathway.
2. The antibody or antigen-binding fragment according to item 1, wherein the antibody or antigen-binding fragment is an insurmountable antagonist of CCL1-induced CCR8-Gi₂ signaling.
3. The antibody or antigen-binding fragment according to item 1 or 2, wherein the antibody or antigen-binding fragment has an insurmountable antagonistic activity on the CCL1-CCR8 signaling pathway at a pH of 6.2 to 6.9, preferably at a pH of 6.5.
4. The antibody or antigen-binding fragment according to any one of items 1 to 3, wherein the antibody or antigen-binding fragment has an insurmountable antagonistic activity on CCL1-induced CCR8-Gi₂ signaling at a pH of 6.2 to 6.9, preferably at a pH of 6.5.
5. The antibody or antigen-binding fragment according to any one of items 1 to 4, wherein the antibody or antigen-binding fragment specifically binds to an extracellular domain of human CCR8.
6. The antibody or antigen-binding fragment according to any one of items 1 to 5, wherein the antibody or antigen-binding fragment specifically binds to one or more of the following:
   human CCR8 or a partial sequence thereof, wherein said partial sequence comprises or consists of the amino acid sequence MDYTLDLSVTTVTDYYYPDIFSSPCDAELIQTNG (SEQ ID NO: 2), having a sulfated tyrosine residue in position Y17 and non-sulfated tyrosine residues in the positions Y15 and Y16;
   human CCR8 or a partial sequence thereof, wherein said partial sequence comprises or consists of the amino acid sequence MDYTLDLSVTTVTDYYYPDIFSSPCDAELIQTNG (SEQ ID NO: 3), having sulfated tyrosine residues in the positions Y15 and Y17 and a non-sulfated tyrosine residue in position Y16;
   human CCR8 or a partial sequence thereof, wherein said partial sequence comprises or consists of the amino acid sequence MDYTLDLSVTTVTDYYYPDIFSSPCDAELIQTNG (SEQ ID NO: 4), having sulfated tyrosine residues in the positions Y16 and Y17 and a non-sulfated tyrosine residue in position Y15; and
   human CCR8 or a partial sequence thereof, wherein said partial sequence comprises or consists of the amino acid sequence MDYTLDLSVTTVTDYYYPDIFSSPCDAELIQTNG (SEQ ID NO: 5), having sulfated tyrosine residues in the positions Y15, Y16 and Y17.
7. The antibody or antigen-binding fragment according to any one of items 1 to 6, wherein the antibody or antigen-binding fragment specifically binds to two or more of the following:
   human CCR8 or a partial sequence thereof, wherein said partial sequence comprises or consists of the amino acid sequence MDYTLDLSVTTVTDYYYPDIFSSPCDAELIQTNG (SEQ ID NO: 2), having a sulfated tyrosine residue in position Y17 and non-sulfated tyrosine residues in the positions Y15 and Y16;
   human CCR8 or a partial sequence thereof, wherein said partial sequence comprises or consists of the amino acid sequence MDYTLDLSVTTVTDYYYPDIFSSPCDAELIQTNG (SEQ ID NO: 3), having sulfated tyrosine residues in the positions Y15 and Y17 and a non-sulfated tyrosine residue in position Y16;
   human CCR8 or a partial sequence thereof, wherein said partial sequence comprises or consists of the amino acid sequence MDYTLDLSVTTVTDYYYPDIFSSPCDAELIQTNG (SEQ ID NO: 4), having sulfated tyrosine residues in the positions Y16 and Y17 and a non-sulfated tyrosine residue in position Y15; and
   human CCR8 or a partial sequence thereof, wherein said partial sequence comprises or consists of the amino acid sequence MDYTLDLSVTTVTDYYYPDIFSSPCDAELIQTNG (SEQ ID NO: 5), having sulfated tyrosine residues in the positions Y15, Y16 and Y17.
8. The antibody or antigen-binding fragment according to any one of items 1 to 7, wherein the antibody or antigen-binding fragment specifically binds to all of the following:
   human CCR8 or a partial sequence thereof, wherein said partial sequence comprises or consists of the amino acid sequence MDYTLDLSVTTVTDYYYPDIFSSPCDAELIQTNG (SEQ ID NO: 2), having a sulfated tyrosine residue in position Y17 and non-sulfated tyrosine residues in the positions Y15 and Y16;
   human CCR8 or a partial sequence thereof, wherein said partial sequence comprises or consists of the amino acid sequence MDYTLDLSVTTVTDYYYPDIFSSPCDAELIQTNG (SEQ ID NO: 3), having sulfated tyrosine residues in the positions Y15 and Y17 and a non-sulfated tyrosine residue in position Y16;
   human CCR8 or a partial sequence thereof, wherein said partial sequence comprises or consists of the amino acid sequence MDYTLDLSVTTVTDYYYPDIFSSPCDAELIQTNG (SEQ ID NO: 4), having sulfated tyrosine residues in the positions Y16 and Y17 and a non-sulfated tyrosine residue in position Y15; and
   human CCR8 or a partial sequence thereof, wherein said partial sequence comprises or consists of the amino acid sequence MDYTLDLSVTTVTDYYYPDIFSSPCDAELIQTNG (SEQ ID NO: 5), having sulfated tyrosine residues in the positions Y15, Y16 and Y17.
9. The antibody or antigen-binding fragment according to any one of items 1 to 8, wherein the antibody or antigen-binding fragment inhibits the binding of human CCL1 to human CCR8 with an IC₅₀ of about 20 nM or less, preferably with an IC₅₀ of about 10 nM or less.
10. The antibody or antigen-binding fragment according to any one of items 1 to 9, wherein the antibody or antigen-binding fragment comprises:
   (1) a heavy chain variable domain (VH) comprising a CDR-H1 having the amino acid sequence PYAMN (SEQ ID NO: 6), a CDR-H2 having the amino acid sequence RIRSKSNNYATYYADSVKD (SEQ ID NO: 7), and a CDR-H3 having the amino acid sequence GREAYYRYDGDYYAMDY (SEQ ID NO: 8); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); wherein in each of said CDR-H1, said CDR-H2, said CDR-H3, said CDR-L1, said CDR-L2, and said CDR-L3, a single amino acid residue is optionally replaced by a different amino acid residue; or
   (2) a heavy chain variable domain (VH) comprising a CDR-H1 having the amino acid sequence TYAMN (SEQ ID NO: 12), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); wherein in each of said CDR-H1, said CDR-H2, said CDR-H3, said CDR-L1, said CDR-L2, and said CDR-L3, a single amino acid residue is optionally replaced by a different amino acid residue; or
   (3) a heavy chain variable domain (VH) comprising a CDR-H1 having the amino acid sequence EYTIH (SEQ ID NO: 14), a CDR-H2 having the amino acid sequence GINPNGDTRYDQKFKG (SEQ ID NO: 15), and a CDR-H3 having the amino acid sequence VARFYGISPYAMDY (SEQ ID NO: 16); and
      a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQSTHVPYT (SEQ ID NO: 19); wherein in each of said CDR-H1, said CDR-H2, said CDR-H3, said CDR-L1, said CDR-L2, and said CDR-L3, a single amino acid residue is optionally replaced by a different amino acid residue; or
   (4) a heavy chain variable domain (VH) comprising a CDR-H1 having the amino acid sequence AYAMN (SEQ ID NO: 20), a CDR-H2 having the amino acid sequence RIRSKSNDYATYYGDSVKD (SEQ ID NO: 21), and a CDR-H3 having the amino acid sequence GREAYYRYDGGYYAMDY (SEQ ID NO: 13); and
      a light chain variable domain (VL) comprising a CDR-L1 having the amino acid sequence TSSKSLLHSNGNTYLY (SEQ ID NO: 22), a CDR-L2 having the amino acid sequence RMSNLAS (SEQ ID NO: 10), and a CDR-L3 having the amino acid sequence MQHREYPFT (SEQ ID NO: 23); wherein in each of said CDR-H1, said CDR-H2, said CDR-H3, said CDR-L1, said CDR-L2, and said CDR-L3, a single amino acid residue is optionally replaced by a different amino acid residue; or
   (5) a heavy chain variable domain (VH) comprising a CDR-H1 having the amino acid sequence DYVVT (SEQ ID NO: 24), a CDR-H2 having the amino acid sequence VIWGGGNTYYNSDLKS (SEQ ID NO: 25), and a CDR-H3 having the amino acid sequence RHRDYALDY (SEQ ID NO: 26); and
      a light chain variable domain (VL) comprising a CDR-L1 having the amino acid sequence RSSQSLVYSNGNTYLH (SEQ ID NO: 27), a CDR-L2 having the amino acid sequence KVSNRFS (SEQ ID NO: 18), and a CDR-L3 having the amino acid sequence SQSTYVPPT (SEQ ID NO: 28); wherein in each of said CDR-H1, said CDR-H2, said CDR-H3, said CDR-L1, said CDR-L2, and said CDR-L3, a single amino acid residue is optionally replaced by a different amino acid residue; or
   (6) a heavy chain variable domain (VH) comprising a CDR-H1 having the amino acid sequence DYTLH (SEQ ID NO: 29), a CDR-H2 having the amino acid sequence GITPKNGDTRYDPRFKD (SEQ ID NO: 30), and a CDR-H3 having the amino acid sequence VARFYGVSPYAMDY (SEQ ID NO: 31); and
      a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQTTHVPYT (SEQ ID NO: 32); wherein in each of said CDR-H1, said CDR-H2, said CDR-H3, said CDR-L1, said CDR-L2, and said CDR-L3, a single amino acid residue is optionally replaced by a different amino acid residue; or
   (7) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PDGFSPFVY (SEQ ID NO: 35); and
      a light chain variable domain (VL) comprising a CDR-L1 having the amino acid sequence KASQDINSYLS (SEQ ID NO: 36), a CDR-L2 having the amino acid sequence RANRLVD (SEQ ID NO: 37), and a CDR-L3 having the amino acid sequence LQYGEFPPT (SEQ ID NO: 38);
      wherein in each of said CDR-H1, said CDR-H2, said CDR-H3, said CDR-L1, said CDR-L2, and said CDR-L3, a single amino acid residue is optionally replaced by a different amino acid residue; or
   (8) a heavy chain variable domain (VH) comprising a CDR-H1 having the amino acid sequence AYAMN (SEQ ID NO: 20), a CDR-H2 having the amino acid sequence RIRSKSNNYATYYGDSVKD (SEQ ID NO: 39), and a CDR-H3 having the amino acid sequence GREAYYRYDGDYYAMDY (SEQ ID NO: 8); and
      a light chain variable domain (VL) comprising a CDR-L1 having the amino acid sequence KSSQSLLNSSNQKNYLA (SEQ ID NO: 40), a CDR-L2 having the amino acid sequence FASTRES (SEQ ID NO: 41), and a CDR-L3 having the amino acid sequence QQHYSTPYT (SEQ ID NO: 42); wherein in each of said CDR-H1, said CDR-H2, said CDR-H3, said CDR-L1, said CDR-L2, and said CDR-L3, a single amino acid residue is optionally replaced by a different amino acid residue; or
   (9) a heavy chain variable domain (VH) comprising a CDR-H1 having the amino acid sequence AYAMN (SEQ ID NO: 20), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); wherein in each of said CDR-H1, said CDR-H2, said CDR-H3, said CDR-L1, said CDR-L2, and said CDR-L3, a single amino acid residue is optionally replaced by a different amino acid residue; or
   (10) a heavy chain variable domain (VH) comprising a CDR-H1 having the amino acid sequence EYTIH (SEQ ID NO: 14), a CDR-H2 having the amino acid sequence GINPNNGNTRYDQKFKG (SEQ ID NO: 43), and a CDR-H3 having the amino acid sequence VARSSGSGPYAMDY (SEQ ID NO: 44); and
      a light chain variable domain (VL) comprising a CDR-L1 having the amino acid sequence RCTQSLLHTNGDTYLH (SEQ ID NO: 45), a CDR-L2 having the amino acid sequence KVSNRFS (SEQ ID NO: 18), and a CDR-L3 having the amino acid sequence SQSTHVPYT (SEQ ID NO: 19); wherein in each of said CDR-H1, said CDR-H2, said CDR-H3, said CDR-L1, said CDR-L2, and said CDR-L3, a single amino acid residue is optionally replaced by a different amino acid residue; or
   (11) a heavy chain variable domain (VH) comprising a CDR-H1 having the amino acid sequence GYNMH (SEQ ID NO: 46), a CDR-H2 having the amino acid sequence AISPGKGDTSYNLKFKG (SEQ ID NO: 47), and a CDR-H3 having the amino acid sequence SGGTPFAY (SEQ ID NO: 48); and
      a light chain variable domain (VL) comprising a CDR-L1 having the amino acid sequence RSSQSLVHSNGDTYLY (SEQ ID NO: 49), a CDR-L2 having the amino acid sequence KVSNRFS (SEQ ID NO: 18), and a CDR-L3 having the amino acid sequence SQSTHVPYT (SEQ ID NO: 19); wherein in each of said CDR-H1, said CDR-H2, said CDR-H3, said CDR-L1, said CDR-L2, and said CDR-L3, a single amino acid residue is optionally replaced by a different amino acid residue; or
   (12) a heavy chain variable domain (VH) comprising a CDR-H1 having the amino acid sequence ANAMN (SEQ ID NO: 50), a CDR-H2 having the amino acid sequence RIRSKSNNYATYYADSVKD (SEQ ID NO: 7), and a CDR-H3 having the amino acid sequence GSDNYIFYAMDY (SEQ ID NO: 51); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); wherein in each of said CDR-H1, said CDR-H2, said CDR-H3, said CDR-L1, said CDR-L2, and said CDR-L3, a single amino acid residue is optionally replaced by a different amino acid residue; or
   (13) a heavy chain variable domain (VH) comprising a CDR-H1 having the amino acid sequence TNAMN (SEQ ID NO: 52), a CDR-H2 having the amino acid sequence RIRSKSNYYATYYADSVKD (SEQ ID NO: 53), and a CDR-H3 having the amino acid sequence GREMGNYYSMDY (SEQ ID NO: 54); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); wherein in each of said CDR-H1, said CDR-H2, said CDR-H3, said CDR-L1, said CDR-L2, and said CDR-L3, a single amino acid residue is optionally replaced by a different amino acid residue; or
   (14) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PTYPGSSGFAY (SEQ ID NO: 55); and
      A light chain variable domain (VL) comprising a CDR-L1 having the amino acid sequence RASQDISNYLN (SEQ ID NO: 56), a CDR-L2 having the amino acid sequence YTSRLHS (SEQ ID NO: 57), and a CDR-L3 having the amino acid sequence QQGNKFPPT (SEQ ID NO: 58);
      wherein in each of said CDR-H1, said CDR-H2, said CDR-H3, said CDR-L1, said CDR-L2, and said CDR-L3, a single amino acid residue is optionally replaced by a different amino acid residue; or
   (15) a heavy chain variable domain (VH) comprising a CDR-H1 having the amino acid sequence EYTMH (SEQ ID NO: 59), a CDR-H2 having the amino acid sequence GINPNNGDTNYNQKFMG (SEQ ID NO: 60), and a CDR-H3 having the amino acid sequence RLLRRGAMDY (SEQ ID NO: 61); and
      a light chain variable domain (VL) comprising a CDR-L1 having the amino acid sequence RASENVEYYGTSLMQ (SEQ ID NO: 62), a CDR-L2 having the amino acid sequence AASNVDS (SEQ ID NO: 63), and a CDR-L3 having the amino acid sequence QQSRKVPWT (SEQ ID NO: 64); wherein in each of said CDR-H1, said CDR-H2, said CDR-H3, said CDR-L1, said CDR-L2, and said CDR-L3, a single amino acid residue is optionally replaced by a different amino acid residue; or
   (16) a heavy chain variable domain (VH) comprising a CDR-H1 having the amino acid sequence GSYMH (SEQ ID NO: 65), a CDR-H2 having the amino acid sequence RINPYNGATSYNQNFKD (SEQ ID NO: 66), and a CDR-H3 having the amino acid sequence TLLRLLDY (SEQ ID NO: 67); and
      a light chain variable domain (VL) comprising a CDR-L1 having the amino acid sequence RSSKSLLHSNGITYLY (SEQ ID NO: 68), a CDR-L2 having the amino acid sequence QMSNLAS (SEQ ID NO: 69), and a CDR-L3 having the amino acid sequence AQNLELPWT (SEQ ID NO: 70); wherein in each of said CDR-H1, said CDR-H2, said CDR-H3, said CDR-L1, said CDR-L2, and said CDR-L3, a single amino acid residue is optionally replaced by a different amino acid residue; or
   (17) a heavy chain variable domain (VH) comprising a CDR-H1 having the amino acid sequence SYWMH (SEQ ID NO: 71), a CDR-H2 having the amino acid sequence NIWPGSASTNYDEKFKN (SEQ ID NO: 72), and a CDR-H3 having the amino acid sequence GGKGAMDY (SEQ ID NO: 73); and
      a light chain variable domain (VL) comprising a CDR-L1 having the amino acid sequence RSSQSLENSYGNTYLN (SEQ ID NO: 74), a CDR-L2 having the amino acid sequence RVSNRFS (SEQ ID NO: 75), and a CDR-L3 having the amino acid sequence LQVTHVPPT (SEQ ID NO: 76); wherein in each of said CDR-H1, said CDR-H2, said CDR-H3, said CDR-L1, said CDR-L2, and said CDR-L3, a single amino acid residue is optionally replaced by a different amino acid residue; or
   (18) a heavy chain variable domain (VH) comprising a CDR-H1 having the amino acid sequence TYAMH (SEQ ID NO: 77), a CDR-H2 having the amino acid sequence HINPSSGYSNYNQKFKD (SEQ ID NO: 78), and a CDR-H3 having the amino acid sequence SEVRRGYFDV (SEQ ID NO: 79); and
      a light chain variable domain (VL) comprising a CDR-L1 having the amino acid sequence RTSQDIRNYLN (SEQ ID NO: 80), a CDR-L2 having the amino acid sequence YTSRLHS (SEQ ID NO: 57), and a CDR-L3 having the amino acid sequence QQGNTLPPT (SEQ ID NO: 81);
      wherein in each of said CDR-H1, said CDR-H2, said CDR-H3, said CDR-L1, said CDR-L2, and said CDR-L3, a single amino acid residue is optionally replaced by a different amino acid residue; or
   (19) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDV (SEQ ID NO: 124); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11);
      wherein in each of said CDR-H1, said CDR-H2, said CDR-H3, said CDR-L1, said CDR-L2, and said CDR-L3, a single amino acid residue is optionally replaced by a different amino acid residue; or
   (20) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23);
      wherein in each of said CDR-H1, said CDR-H2, said CDR-H3, said CDR-L1, said CDR-L2, and said CDR-L3, a single amino acid residue is optionally replaced by a different amino acid residue; or
   (21) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
      A light chain variable domain (VL) comprising a CDR-L1 having the amino acid sequence KSLLHSNGNTY (SEQ ID NO: 125), a CDR-L2 having the amino acid sequence RVS, and a CDR-L3 having the amino acid sequence MQHREYPFT (SEQ ID NO: 23);
      wherein in each of said CDR-H1, said CDR-H2, said CDR-H3, said CDR-L1, said CDR-L2, and said CDR-L3, a single amino acid residue is optionally replaced by a different amino acid residue; or
   (22) a heavy chain variable domain (VH) comprising a CDR-H1 having the amino acid sequence GYTFTEYT (SEQ ID NO: 128), a CDR-H2 having the amino acid sequence INPNNGNT (SEQ ID NO: 129), and a CDR-H3 having the amino acid sequence ARVARSSGSGPYAMDY (SEQ ID NO: 130); and
      a light chain variable domain (VL) comprising a CDR-L1 having the amino acid sequence QSLLHTNGDTY (SEQ ID NO: 131), a CDR-L2 having the amino acid sequence KVS, and a CDR-L3 having the amino acid sequence SQSTHVPYT (SEQ ID NO: 19);
      wherein in each of said CDR-H1, said CDR-H2, said CDR-H3, said CDR-L1, said CDR-L2, and said CDR-L3, a single amino acid residue is optionally replaced by a different amino acid residue; or
   (23) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDY (SEQ ID NO: 133); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23);
      wherein in each of said CDR-H1, said CDR-H2, said CDR-H3, said CDR-L1, said CDR-L2, and said CDR-L3, a single amino acid residue is optionally replaced by a different amino acid residue; or
   (24) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDY (SEQ ID NO: 133); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11);
      wherein in each of said CDR-H1, said CDR-H2, said CDR-H3, said CDR-L1, said CDR-L2, and said CDR-L3, a single amino acid residue is optionally replaced by a different amino acid residue; or
   (25) a heavy chain variable domain (VH) comprising a CDR-H1 having the amino acid sequence GFTFSNYR (SEQ ID NO: 134), a CDR-H2 having the amino acid sequence IKVKSDNYGA (SEQ ID NO: 135), and a CDR-H3 having the amino acid sequence SSPTYPGSSGFAY (SEQ ID NO: 136); and
      a light chain variable domain (VL) comprising a CDR-L1 having the amino acid sequence QDISNY (SEQ ID NO: 137), a CDR-L2 having the amino acid sequence YTS, and a CDR-L3 having the amino acid sequence QQGNKFPPT (SEQ ID NO: 58);
      wherein in each of said CDR-H1, said CDR-H2, said CDR-H3, said CDR-L1, said CDR-L2, and said CDR-L3, a single amino acid residue is optionally replaced by a different amino acid residue.
11. The antibody or antigen-binding fragment according to any one of items 1 to 10, wherein the antibody or antigen-binding fragment comprises:
   (1) a heavy chain variable domain (VH) comprising a CDR-H1 having the amino acid sequence PYAMN (SEQ ID NO: 6), a CDR-H2 having the amino acid sequence RIRSKSNNYATYYADSVKD (SEQ ID NO: 7), and a CDR-H3 having the amino acid sequence GREAYYRYDGDYYAMDY (SEQ ID NO: 8); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
   (2) a heavy chain variable domain (VH) comprising a CDR-H1 having the amino acid sequence TYAMN (SEQ ID NO: 12), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
   (3) a heavy chain variable domain (VH) comprising a CDR-H1 having the amino acid sequence EYTIH (SEQ ID NO: 14), a CDR-H2 having the amino acid sequence GINPNGDTRYDQKFKG (SEQ ID NO: 15), and a CDR-H3 having the amino acid sequence VARFYGISPYAMDY (SEQ ID NO: 16); and
      a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQSTHVPYT (SEQ ID NO: 19); or
   (4) a heavy chain variable domain (VH) comprising a CDR-H1 having the amino acid sequence AYAMN (SEQ ID NO: 20), a CDR-H2 having the amino acid sequence RIRSKSNDYATYYGDSVKD (SEQ ID NO: 21), and a CDR-H3 having the amino acid sequence GREAYYRYDGGYYAMDY (SEQ ID NO: 13); and
      a light chain variable domain (VL) comprising a CDR-L1 having the amino acid sequence TSSKSLLHSNGNTYLY (SEQ ID NO: 22), a CDR-L2 having the amino acid sequence RMSNLAS (SEQ ID NO: 10), and a CDR-L3 having the amino acid sequence MQHREYPFT (SEQ ID NO: 23); or
   (5) a heavy chain variable domain (VH) comprising a CDR-H1 having the amino acid sequence DYVVT (SEQ ID NO: 24), a CDR-H2 having the amino acid sequence VIWGGGNTYYNSDLKS (SEQ ID NO: 25), and a CDR-H3 having the amino acid sequence RHRDYALDY (SEQ ID NO: 26); and
      a light chain variable domain (VL) comprising a CDR-L1 having the amino acid sequence RSSQSLVYSNGNTYLH (SEQ ID NO: 27), a CDR-L2 having the amino acid sequence KVSNRFS (SEQ ID NO: 18), and a CDR-L3 having the amino acid sequence SQSTYVPPT (SEQ ID NO: 28); or
   (6) a heavy chain variable domain (VH) comprising a CDR-H1 having the amino acid sequence DYTLH (SEQ ID NO: 29), a CDR-H2 having the amino acid sequence GITPKNGDTRYDPRFKD (SEQ ID NO: 30), and a CDR-H3 having the amino acid sequence VARFYGVSPYAMDY (SEQ ID NO: 31); and
      a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQTTHVPYT (SEQ ID NO: 32); or
   (7) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PDGFSPFVY (SEQ ID NO: 35); and
      a light chain variable domain (VL) comprising a CDR-L1 having the amino acid sequence KASQDINSYLS (SEQ ID NO: 36), a CDR-L2 having the amino acid sequence RANRLVD (SEQ ID NO: 37), and a CDR-L3 having the amino acid sequence LQYGEFPPT (SEQ ID NO: 38); or
   (8) a heavy chain variable domain (VH) comprising a CDR-H1 having the amino acid sequence AYAMN (SEQ ID NO: 20), a CDR-H2 having the amino acid sequence RIRSKSNNYATYYGDSVKD (SEQ ID NO: 39), and a CDR-H3 having the amino acid sequence GREAYYRYDGDYYAMDY (SEQ ID NO: 8); and
      a light chain variable domain (VL) comprising a CDR-L1 having the amino acid sequence KSSQSLLNSSNQKNYLA (SEQ ID NO: 40), a CDR-L2 having the amino acid sequence FASTRES (SEQ ID NO: 41), and a CDR-L3 having the amino acid sequence QQHYSTPYT (SEQ ID NO: 42); or
   (9) a heavy chain variable domain (VH) comprising a CDR-H1 having the amino acid sequence AYAMN (SEQ ID NO: 20), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
   (10) a heavy chain variable domain (VH) comprising a CDR-H1 having the amino acid sequence EYTIH (SEQ ID NO: 14), a CDR-H2 having the amino acid sequence GINPNNGNTRYDQKFKG (SEQ ID NO: 43), and a CDR-H3 having the amino acid sequence VARSSGSGPYAMDY (SEQ ID NO: 44); and
      a light chain variable domain (VL) comprising a CDR-L1 having the amino acid sequence RCTQSLLHTNGDTYLH (SEQ ID NO: 45), a CDR-L2 having the amino acid sequence KVSNRFS (SEQ ID NO: 18), and a CDR-L3 having the amino acid sequence SQSTHVPYT (SEQ ID NO: 19); or
   (11) a heavy chain variable domain (VH) comprising a CDR-H1 having the amino acid sequence GYNMH (SEQ ID NO: 46), a CDR-H2 having the amino acid sequence AISPGKGDTSYNLKFKG (SEQ ID NO: 47), and a CDR-H3 having the amino acid sequence SGGTPFAY (SEQ ID NO: 48); and
      a light chain variable domain (VL) comprising a CDR-L1 having the amino acid sequence RSSQSLVHSNGDTYLY (SEQ ID NO: 49), a CDR-L2 having the amino acid sequence KVSNRFS (SEQ ID NO: 18), and a CDR-L3 having the amino acid sequence SQSTHVPYT (SEQ ID NO: 19); or
   (12) a heavy chain variable domain (VH) comprising a CDR-H1 having the amino acid sequence ANAMN (SEQ ID NO: 50), a CDR-H2 having the amino acid sequence RIRSKSNNYATYYADSVKD (SEQ ID NO: 7), and a CDR-H3 having the amino acid sequence GSDNYIFYAMDY (SEQ ID NO: 51); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
   (13) a heavy chain variable domain (VH) comprising a CDR-H1 having the amino acid sequence TNAMN (SEQ ID NO: 52), a CDR-H2 having the amino acid sequence RIRSKSNYYATYYADSVKD (SEQ ID NO: 53), and a CDR-H3 having the amino acid sequence GREMGNYYSMDY (SEQ ID NO: 54); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
   (14) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PTYPGSSGFAY (SEQ ID NO: 55); and
      a light chain variable domain (VL) comprising a CDR-L1 having the amino acid sequence RASQDISNYLN (SEQ ID NO: 56), a CDR-L2 having the amino acid sequence YTSRLHS (SEQ ID NO: 57), and a CDR-L3 having the amino acid sequence QQGNKFPPT (SEQ ID NO: 58); or
   (15) a heavy chain variable domain (VH) comprising a CDR-H1 having the amino acid sequence EYTMH (SEQ ID NO: 59), a CDR-H2 having the amino acid sequence GINPNNGDTNYNQKFMG (SEQ ID NO: 60), and a CDR-H3 having the amino acid sequence RLLRRGAMDY (SEQ ID NO: 61); and
      a light chain variable domain (VL) comprising a CDR-L1 having the amino acid sequence RASENVEYYGTSLMQ (SEQ ID NO: 62), a CDR-L2 having the amino acid sequence AASNVDS (SEQ ID NO: 63), and a CDR-L3 having the amino acid sequence QQSRKVPWT (SEQ ID NO: 64); or
   (16) a heavy chain variable domain (VH) comprising a CDR-H1 having the amino acid sequence GSYMH (SEQ ID NO: 65), a CDR-H2 having the amino acid sequence RINPYNGATSYNQNFKD (SEQ ID NO: 66), and a CDR-H3 having the amino acid sequence TLLRLLDY (SEQ ID NO: 67); and
      a light chain variable domain (VL) comprising a CDR-L1 having the amino acid sequence RSSKSLLHSNGITYLY (SEQ ID NO: 68), a CDR-L2 having the amino acid sequence QMSNLAS (SEQ ID NO: 69), and a CDR-L3 having the amino acid sequence AQNLELPWT (SEQ ID NO: 70); or
   (17) a heavy chain variable domain (VH) comprising a CDR-H1 having the amino acid sequence SYWMH (SEQ ID NO: 71), a CDR-H2 having the amino acid sequence NIWPGSASTNYDEKFKN (SEQ ID NO: 72), and a CDR-H3 having the amino acid sequence GGKGAMDY (SEQ ID NO: 73); and
      a light chain variable domain (VL) comprising a CDR-L1 having the amino acid sequence RSSQSLENSYGNTYLN (SEQ ID NO: 74), a CDR-L2 having the amino acid sequence RVSNRFS (SEQ ID NO: 75), and a CDR-L3 having the amino acid sequence LQVTHVPPT (SEQ ID NO: 76); or
   (18) a heavy chain variable domain (VH) comprising a CDR-H1 having the amino acid sequence TYAMH (SEQ ID NO: 77), a CDR-H2 having the amino acid sequence HINPSSGYSNYNQKFKD (SEQ ID NO: 78), and a CDR-H3 having the amino acid sequence SEVRRGYFDV (SEQ ID NO: 79); and
      a light chain variable domain (VL) comprising a CDR-L1 having the amino acid sequence RTSQDIRNYLN (SEQ ID NO: 80), a CDR-L2 having the amino acid sequence YTSRLHS (SEQ ID NO: 57), and a CDR-L3 having the amino acid sequence QQGNTLPPT (SEQ ID NO: 81); or
   (19) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDV (SEQ ID NO: 124); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11); or
   (20) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23); or
   (21) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
      a light chain variable domain (VL) comprising a CDR-L1 having the amino acid sequence KSLLHSNGNTY (SEQ ID NO: 125), a CDR-L2 having the amino acid sequence RVS, and a CDR-L3 having the amino acid sequence MQHREYPFT (SEQ ID NO: 23); or
   (22) a heavy chain variable domain (VH) comprising a CDR-H1 having the amino acid sequence GYTFTEYT (SEQ ID NO: 128), a CDR-H2 having the amino acid sequence INPNNGNT (SEQ ID NO: 129), and a CDR-H3 having the amino acid sequence ARVARSSGSGPYAMDY (SEQ ID NO: 130); and
      a light chain variable domain (VL) comprising a CDR-L1 having the amino acid sequence QSLLHTNGDTY (SEQ ID NO: 131), a CDR-L2 having the amino acid sequence KVS, and a CDR-L3 having the amino acid sequence SQSTHVPYT (SEQ ID NO: 19); or
   (23) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDY (SEQ ID NO: 133); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23); or
   (24) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDY (SEQ ID NO: 133); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11); or
   (25) a heavy chain variable domain (VH) comprising a CDR-H1 having the amino acid sequence GFTFSNYR (SEQ ID NO: 134), a CDR-H2 having the amino acid sequence IKVKSDNYGA (SEQ ID NO: 135), and a CDR-H3 having the amino acid sequence SSPTYPGSSGFAY (SEQ ID NO: 136); and
      a light chain variable domain (VL) comprising a CDR-L1 having the amino acid sequence QDISNY (SEQ ID NO: 137), a CDR-L2 having the amino acid sequence YTS, and a CDR-L3 having the amino acid sequence QQGNKFPPT (SEQ ID NO: 58).
12. The antibody or antigen-binding fragment according to any one of items 1 to 11, wherein the antibody or antigen-binding fragment comprises:
   (1) a heavy chain variable domain (VH) having an amino acid sequence with at least 80% sequence identity to the amino acid sequence MLLGLKWVFFVVFYQGVLCEVQLLESGGGLVQPKGSLKLSCAASGFTFNPYAMNWVRQAPGRG LEWVARIRSKSNNYATYYADSVKDRFTISRDDSQDMLYLQMNNLKTEDTAMYYCVRGREAYYRY DGDYYAMDYWGQGTSVTVSS (SEQ ID NO: 82); and
      a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
   (2) a heavy chain variable domain (VH) having an amino acid sequence with at least 80% sequence identity to the amino acid sequence MLLGLKWVFFVVFYQGVHCEVQLVESGGGLVQPKGSLKLSCAASGFTFNTYAMNWVRQAPGK GLEWVARIRSKSNNYATYYADSVKDRFTISRDDSQSMLYLQMNNLKTEDTAMYYCVRGREAYYR YDGGYYAMDYWGQGTSVTVSS (SEQ ID NO: 84); and
      a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
   (3) a heavy chain variable domain (VH) having an amino acid sequence with at least 80% sequence identity to the amino acid sequence MGWSWIFLFLLSGTAGVLSKVQLQQSGPELVKPGASVKISCKTSGYTFTEYTIHWVQQIHGKSPE WIGGINPNGDTRYDQKFKGKATLTIDKSSSTAYMELRSLTSEDSAVYYCARVARFYGISPYAMDY WGQGTSVTVSS (SEQ ID NO: 85); and
      a light chain variable domain (VL) having an amino acid sequence with at least 80% sequence identity to the amino acid sequence MKLPVRLLVLMFWIPASSSDVVMTQTPLSLPVSLGDQASISCRCTQSLLHSNGDTYLHWYLQKP GQSPNLLIYKVSNRFSGVPDRFSGSGSGTDFTLKINRVEAEDLGVYFCSQSTHVPYTFGGGTNLE IK (SEQ ID NO: 86); or
   (4) a heavy chain variable domain (VH) having an amino acid sequence with at least 80% sequence identity to the amino acid sequence MLLGLKWVFFVVFYQGVHCEVQLVESGGGLVQPTGSLRLSCAASGFTFNAYAMNWVRQAPGK GLEWVARIRSKSNDYATYYGDSVKDRFTISRDDSQSMLYLQMNNLKTEDTAMYYCVRGREAYY RYDGGYYAMDYWGQGTSVTVSS (SEQ ID NO: 87); and
      a light chain variable domain (VL) having an amino acid sequence with at least 80% sequence identity to the amino acid sequence MRCLAEFLGLLVLWIPGAIGDIVMTQAAPSVSVTPGESVSISCTSSKSLLHSNGNTYLYWFLQRPG QSPQLLIYRMSNLASGVPDRFSGSGSGTAFTLRISRVEAEDVGVYYCMQHREYPFTFGSGTKLEI K (SEQ ID NO: 88); or
   (5) a heavy chain variable domain (VH) having an amino acid sequence with at least 80% sequence identity to the amino acid sequence MAVLGLLLCLVTFPSCVLSQVQLKESGPGLVAPSQSLSITCTVSGLSMNDYVVTWIRQPPGKGLE WLGVIWGGGNTYYNSDLKSRLSITKDNSKSQVFFKMSSLQTDDTAVYYCARRHRDYALDYWGQ GISVTVSS (SEQ ID NO: 89); and
      a light chain variable domain (VL) having an amino acid sequence with at least 80% sequence identity to the amino acid sequence MKLPVRLLVLMFWIPASSSDVVMTQTPLSLPVSLGDQASISCRSSQSLVYSNGNTYLHWYLQKP GQSPKLLIYKVSNRFSGVPDRFSGSGSGTDFTLKISRVEAEDLGVYFCSQSTYVPPTFGGGTKLEI K (SEQ ID NO: 90); or
   (6) a heavy chain variable domain (VH) having an amino acid sequence with at least 80% sequence identity to the amino acid sequence MGWSWIFLFLLSGTAGVLSEVQLQQSGPELLKPGTSVKISCTTSGYTFSDYTLHWVKQSHGKSL EWIGGITPKNGDTRYDPRFKDKATLTIDKSSSAAYMELRSLTSEDSAVYYCARVARFYGVSPYAM DYWGQGASVTVSS (SEQ ID NO: 91); and
      a light chain variable domain (VL) having an amino acid sequence with at least 80% sequence identity to the amino acid sequence MKLPVRLLVLMFWIPVSNSDVVMTQTPLSLPVSLGDQASISCRCTQSLLHSNGDTYLHWYLQKP GQSPKLLIYKVSNRFSGVPDRFSGSGSGTDFTLEITRVEAEDLGVYFCSQTTHVPYTFGGGTKLEI K (SEQ ID NO: 92); or
   (7) a heavy chain variable domain (VH) having an amino acid sequence with at least 80% sequence identity to the amino acid sequence MELGLSWVFLVALLNGVQCQVHLVETGGGLVRPGNSLKLSCVTSGFTLSNYRMHWLRQPPGKR LEWIAVIKVKSDNYGANYAESVKGRFTISRDDAKSSVYLQMNRLREEDTATYYCSRPDGFSPFVY WGQGTLVTVSA (SEQ ID NO: 93); and
      a light chain variable domain (VL) having an amino acid sequence with at least 80% sequence identity to the amino acid sequence MDMRTPAQFLGILLLWFPGIKCDIKMTQSPSSMYASLGERVTITCKASQDINSYLSWFQQKPGKS PKTLIYRANRLVDGVPSRFSGSGSGQDYFLTISSLEYEDMGIYYCLQYGEFPPTFGAGTKLELK (SEQ ID NO: 94); or
   (8) a heavy chain variable domain (VH) having an amino acid sequence with at least 80% sequence identity to the amino acid sequence MLLGLKWVFFVVFYQGVHCEVQLVESGGGLVQPKGSLRLSCAASGFTFNAYAMNWVRQAPGK GLEWVARIRSKSNNYATYYGDSVKDRFTISRDDSQTMLYLQMNNLKTEDTAMYYCVRGREAYYR YDGDYYAMDYWGQGTSVTVSS (SEQ ID NO: 95); and
      a light chain variable domain (VL) having an amino acid sequence with at least 80% sequence identity to the amino acid sequence MESQTQVLMFLLLWVSGACADIVMTQSPSSLAMSVGQKVTMSCKSSQSLLNSSNQKNYLAWYQ QKPGQSPKLLVYFASTRESGVPDRFIGSGSGTDFTLTISSVQAEDLADYFCQQHYSTPYTFGGGT KLEIK (SEQ ID NO: 96); or
   (9) a heavy chain variable domain (VH) having an amino acid sequence with at least 80% sequence identity to the amino acid sequence MLLGLKWVFFVVFYQGVHCEVQLVESGGGLVQPKGSLKLSCAASGFTFNAYAMNWVRQAPGK GLEWLARIRSKSNNYATYYADSVKDRFTISRDDSQSMFYLQMNNLKSEDTAMYYCVRGREAYYR YDGGYYAMDYWGQGTSVTVSS (SEQ ID NO: 97); and
      a light chain variable domain (VL) having an amino acid sequence with at least 80% sequence identity to the amino acid sequence MRCLAEFLGLLVLWIPGAIGEIVMTQAALSAPVTPGESVSMSCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 98); or
   (10) a heavy chain variable domain (VH) having an amino acid sequence with at least 80% sequence identity to the amino acid sequence MGWSWIFLFLLSGTAGVLSEVQLQQSGPELVKPGASVKISCKTSGYTFTEYTIHWVKQSHGESLE WVGGINPNNGNTRYDQKFKGKATLTIDKSSSPAYMELRSLTSEDSAVYYCARVARSSGSGPYAM DYWGQGTSVTVSS (SEQ ID NO: 99); and
      a light chain variable domain (VL) having an amino acid sequence with at least 80% sequence identity to the amino acid sequence MKLPVRLLVLMFWIPVSSSDVVMTQTPLSLPVSLGDQASISCRCTQSLLHTNGDTYLHWYLQKPG QSPKLLIYKVSNRFSGVPDRFSGSGSGTDFTLKISRVEAEDLGVYFCSQSTHVPYTFGGGTKLEIK (SEQ ID NO: 100); or
   (11) a heavy chain variable domain (VH) having an amino acid sequence with at least 80% sequence identity to the amino acid sequence MGWSCIILFLVATATDVHSQVQLQQPGAELVKPGASVKMSCKATGYTFTGYNMHWVKQTPGQG LEWIGAISPGKGDTSYNLKFKGKATLTTDKSSSTAYMQLSSLTSADSAVYYCARSGGTPFAYWG QGTLVTVSA (SEQ ID NO: 101); and
      a light chain variable domain (VL) having an amino acid sequence with at least 80% sequence identity to the amino acid sequence MKLPVRLLVLMFWIPASSSDVVMTQTPLSLPVSLGDQASISCRSSQSLVHSNGDTYLYWYLQKP GQSPKLLIYKVSNRFSGVPDRFSGSGSGTDFTLKINRVEAEDLGVYFCSQSTHVPYTFGGGTKLE IK (SEQ ID NO: 102); or
   (12) a heavy chain variable domain (VH) having an amino acid sequence with at least 80% sequence identity to the amino acid sequence MLLGLKWVFFVVFYQGVHCEVQLVETGGGLVQPKGSLKLSCAASGFTFNANAMNWVRQAPGK GLEWVARIRSKSNNYATYYADSVKDRFTISRDDSQSMLYLQMNNLKTEDTAMYYCVRGSDNYIF YAMDYWGQGTSVTVSS (SEQ ID NO: 103); and
      a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGGGTKL EIK (SEQ ID NO: 104); or
   (13) a heavy chain variable domain (VH) having an amino acid sequence with at least 80% sequence identity to the amino acid sequence MLLGLKWVFFVVFYQGVHCEVQLVETGGGLVQPKGSLKLSCAASGFTFNTNAMNWVRQAPGK GLEWVARIRSKSNYYATYYADSVKDRFTISRDDSQSMLYLQMNNLKTEDTAMYYCVRGREMGN YYSMDYWGQGTSVTVSS (SEQ ID NO: 105); and
      a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
   (14) a heavy chain variable domain (VH) having an amino acid sequence with at least 80% sequence identity to the amino acid sequence MELGLSWVFLVALLNGVQCQVQLVETGGGLVRPGNSLKLSCVTSGFTFSNYRMHWLRQPPGKR LEWIAVIKVKSDNYGANYAESVKGRFTISRDDSKSSVYLQVNRLREEDTATYYCSSPTYPGSSGF AYWGQGTLVTVSA (SEQ ID NO: 106); and
      a light chain variable domain (VL) having an amino acid sequence with at least 80% sequence identity to the amino acid sequence MMSSAQFLGLLLLCFQGTRCDIQMTQTTSSLSASLGDRVTISCRASQDISNYLNWYQQKPDGTV KLLIYYTSRLHSGVPSRFSGSGSGTDYSLTISNLEQEDIATYFCQQGNKFPPTFGGGTKLEIN (SEQ ID NO: 107); or
   (15) a heavy chain variable domain (VH) having an amino acid sequence with at least 80% sequence identity to the amino acid sequence MGWSWIFLFLLSGTAGVLSEVQLQQSGPELVKPGASVKISCKTSGYTFTEYTMHWVKQSHGKSL EWIGGINPNNGDTNYNQKFMGKATLTVDKSSSTAYMELRSLTSEDSAVYYCVRRLLRRGAMDY WGQGTSVTVSS (SEQ ID NO: 108); and
      a light chain variable domain (VL) having an amino acid sequence with at least 80% sequence identity to the amino acid sequence MESDTLLLWVLLLWVPGSTGDIVLTQSPASLAVSLGQRATISCRASENVEYYGTSLMQWFQQKP GQPPKLLIYAASNVDSGVPARFSGSGSGTDFSLNIHPVEEDDIAMYFCQQSRKVPWTFGGGTKL DIK (SEQ ID NO: 109); or
   (16) a heavy chain variable domain (VH) having an amino acid sequence with at least 80% sequence identity to the amino acid sequence MGWSWIFLFLLSGTAGVLSEVQLQQSGPELVKPGASVKISCKASVYSFTGSYMHWVKQSHVKSL EWIGRINPYNGATSYNQNFKDKASLTVDKSSSTAYMELHSLTSEDSAVYYCATTLLRLLDYWGQ GTTLTVSS (SEQ ID NO: 110); and
      a light chain variable domain (VL) having an amino acid sequence with at least 80% sequence identity to the amino acid sequence MRFSAQLLGLLVLWIPGSTADIVMTQAAFSNPVTLGTSASISCRSSKSLLHSNGITYLYWYLQKPG QSPQLLIYQMSNLASGVPDRESSSGSGTDFTLRISRVEAEDVGVYYCAQNLELPWTFGGGTKLEI K (SEQ ID NO: 111); or
   (17) a heavy chain variable domain (VH) having an amino acid sequence with at least 80% sequence identity to the amino acid sequence MGWSSIILFLVATASGVHSQVQLQQPGSELVRPGASVKLSCKASGYTFTSYWMHWVKQRPGQG LEWIGNIWPGSASTNYDEKFKNKATLTVDTSSSTAYMQLSSLTSEDSAVYYCIRGGKGAMDYWS QGTSVTVSS (SEQ ID NO: 112); and
      a light chain variable domain (VL) having an amino acid sequence with at least 80% sequence identity to the amino acid sequence MKLPVRLLVLMFWIPVSSSDVVMTQIPLSLPVSLGDQASISCRSSQSLENSYGNTYLNWYLQKPG QSPQLLIYRVSNRFSGVLDRFSGSGSGTDFTLKISRVEAEDLGVYFCLQVTHVPPTFGAGTKLEL K (SEQ ID NO: 113); or
   (18) a heavy chain variable domain (VH) having an amino acid sequence with at least 80% sequence identity to the amino acid sequence MERHWIFLLLLSVTAGVHSQVQVQQSGAELARPGASVKMSCKASGYTFTTYAMHWVKQRPGQ GLEWIGHINPSSGYSNYNQKFKDKATLTADKSSSTAYMQLSSLTSEDSAVYYCARSEVRRGYFD VWGAGTTVTVSS (SEQ ID NO: 114); and
      a light chain variable domain (VL) having an amino acid sequence with at least 80% sequence identity to the amino acid sequence MMSSAQFLGLLLLCFQGTRCDIQMTQTTSSLSASLGDRVTISCRTSQDIRNYLNWYQQKPDGTV KLLISYTSRLHSGVPSRFSGSGSGTDYSLTISNLEQEDFATYFCQQGNTLPPTFGGGTRVEIK (SEQ ID NO: 115); or
   (19) a heavy chain variable domain (VH) having an amino acid sequence with at least 80% sequence identity to the amino acid sequence EVOLVESGGGLVQPGRSLRLSCTASGFTFNTYAMNWVRQAPGKGLEWVARIRSKSNNYATYYA ASVKGRFTISRDDSKSIAYLQMNSLKTEDTAVYYCVRGREAYYRYDGGYYAMDVWGQGTTVTV SS (SEQ ID NO: 138); and
      a light chain variable domain (VL) having an amino acid sequence with at least 80% sequence identity to the amino acid sequence DIVMTQAAPSLPVTPGESASISCRSSKSLLHSNGNTYLYWFLQKPGQSPQLLIYRMSNLASGVPD RFSGSGSGTAFTLKISRVEAEDVGVYYCMQHLEYPFTFGSGTKLEIK (SEQ ID NO: 139); or
   (20) a heavy chain variable domain (VH) having an amino acid sequence with at least 80% sequence identity amino acid to the sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SS (SEQ ID NO: 140); and
      a light chain variable domain (VL) having an amino acid sequence with at least 80% sequence identity to the amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIK (SEQ ID NO: 141); or
   (21) a heavy chain variable domain (VH) having an amino acid sequence with at least 80% sequence identity to the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SS (SEQ ID NO: 140); and
      a light chain variable domain (VL) having an amino acid sequence with at least 80% sequence identity to the amino acid sequence DIVMTQSPLSLPVTPGEPASISCRSSKSLLHSNGNTYLYWFLQKPGQSPQLLIYRVSNLASGVPD RFSGSGSGTDFTLKISRVEAEDVGVYYCMQHREYPFTFGQGTKLEIK (SEQ ID NO: 142); or
   (22) a heavy chain variable domain (VH) having an amino acid sequence with at least 80% sequence identity to the amino acid sequence QVQLVQSGPEVVKPGASVKVSCKTSGYTFTEYTIHWVRQAPGQSLEWVGGINPNNGNTRYDQK FQGRVTITRDKSASTAYMELRSLTSEDTAVYYCARVARSSGSGPYAMDYWGQGTTVTVSS (SEQ ID NO: 143); and
      a light chain variable domain (VL) having an amino acid sequence with at least 80% sequence identity to the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIK (SEQ ID NO: 144); or
   (23) a heavy chain variable domain (VH) having an amino acid sequence with at least 80% sequence identity to the amino acid sequence QVQLQQSGPEVVKPGASVKVSCKTSGYTFTEYTIHWVRQSHGQSLEWVGGINPNNGNTRYDQK FKGRVTITIDKSSSTAYMELRSLTSEDTAVYYCARVARSSGSGPYAMDYWGQGTTVTVSS (SEQ ID NO: 145); and
      a light chain variable domain (VL) having an amino acid sequence with at least 80% sequence identity to the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIK (SEQ ID NO: 144); or
   (24) a heavy chain variable domain (VH) having an amino acid sequence with at least 80% sequence identity to the amino acid sequence QVQLVESGGGVVQPGRSLRLSCAASGFTFSNYRMHWVRQAPGKGLEWIAVIKVKSDNYGANYA DSVKGRFTISRDNSKNTLYLQMNSLRAEDTAVYYCSSPTYPGSSGFAYWGQGTLVTVSS (SEQ ID NO: 146); and
      a light chain variable domain (VL) having an amino acid sequence with at least 80% sequence identity to the amino acid sequence DIQMTQSPSSLSASVGDRVTITCRASQDISNYLNWYQQKPDGTVKLLIYYTSRLHTGVPSRFSGS GSGTDYTLTISNLQQEDIATYFCQQGNKFPPTFGGGTKVEIN (SEQ ID NO: 132); or
   (25) a heavy chain variable domain (VH) having an amino acid sequence with at least 80% sequence identity to the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSS (SEQ ID NO: 147); and
      a light chain variable domain (VL) having an amino acid sequence with at least 80% sequence identity to the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIK (SEQ ID NO: 148); or
   (26) a heavy chain variable domain (VH) having an amino acid sequence with at least 80% sequence identity to the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSS (SEQ ID NO: 149); and
      a light chain variable domain (VL) having an amino acid sequence with at least 80% sequence identity to the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIK (SEQ ID NO: 150); or
   (27) a heavy chain variable domain (VH) having an amino acid sequence with at least 80% sequence identity to the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSS (SEQ ID NO: 151); and
      a light chain variable domain (VL) having an amino acid sequence with at least 80% sequence identity to the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIK (SEQ ID NO: 152); or
   (28) a heavy chain variable domain (VH) having an amino acid sequence with at least 80% sequence identity to the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSA (SEQ ID NO: 153); and
      a light chain variable domain (VL) having an amino acid sequence with at least 80% sequence identity to the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEIN (SEQ ID NO: 154).
13. The antibody or antigen-binding fragment according to any one of items 1 to 12, wherein the antibody or antigen-binding fragment comprises:
   (1) a heavy chain (HC) having the amino acid sequence EVOLVESGGGLVQPGRSLRLSCTASGFTFNTYAMNWVRQAPGKGLEWVARIRSKSNNYATYYA ASVKGRFTISRDDSKSIAYLQMNSLKTEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 159); and
      a light chain (LC) having the amino acid sequence DIVMTQAAPSLPVTPGESASISCRSSKSLLHSNGNTYLYWFLQKPGQSPQLLIYRMSNLASGVPD RFSGSGSGTAFTLKISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 160); or
   (2) a heavy chain the amino acid sequence (HC) having EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 161); and
      a light chain (LC) having the amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 162); or
   (3) a heavy chain (HC) having the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSWVTVPSSSLGTQTYICNVNHKPSNTKVDKKVEPKSCDKTHTCPPCPAPELLGGPDVFLFP PKPKDTLMISRTPEVTCVVVDVSHEDPEVKFNWYVDGVEVHNAKTKPREEQYNSTYRVVSVLTV LHQDWLNGKEYKCKVSNKALPAPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 163); and
      a light chain (LC) having the amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 162); or
   (4) a heavy chain (HC) having the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 161); and
      a light chain (LC) having the amino acid sequence DIVMTQSPLSLPVTPGEPASISCRSSKSLLHSNGNTYLYWFLQKPGQSPQLLIYRVSNLASGVPD RFSGSGSGTDFTLKISRVEAEDVGVYYCMQHREYPFTFGQGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 164); or
   (5) a heavy chain (HC) having the amino acid sequence QVQLVQSGPEVVKPGASVKVSCKTSGYTFTEYTIHWVRQAPGQSLEWVGGINPNNGNTRYDQK FQGRVTITRDKSASTAYMELRSLTSEDTAVYYCARVARSSGSGPYAMDYWGQGTTVTVSSASTK GPSVFPLAPSSKSTSGGTAALGCLVKDYFPEPVTVSWNSGALTSGVHTFPAVLQSSGLYSLSSV VTVPSSSLGTQTYICNVNHKPSNTKVDKKVEPKSCDKTHTCPPCPAPELLGGPSVFLFPPKPKDT LMISRTPEVTCVVVDVSHEDPEVKFNWYVDGVEVHNAKTKPREEQYNSTYRVVSVLTVLHQDWL NGKEYKCKVSNKALPAPIEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 165); and
      a light chain (LC) having the amino acid sequence DWVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO: 166); or
   (6) a heavy chain (HC) having the amino acid sequence QVQLQQSGPEVVKPGASVKVSCKTSGYTFTEYTIHWVRQSHGQSLEWVGGINPNNGNTRYDQK FKGRVTITIDKSSSTAYMELRSLTSEDTAVYYCARVARSSGSGPYAMDYWGQGTTVTVSSASTK GPSVFPLAPSSKSTSGGTAALGCLVKDYFPEPVTVSWNSGALTSGVHTFPAVLQSSGLYSLSSV VTVPSSSLGTQTYICNVNHKPSNTKVDKKVEPKSCDKTHTCPPCPAPELLGGPSVFLFPPKPKDT LMISRTPEVTCVVVDVSHEDPEVKFNWYVDGVEVHNAKTKPREEQYNSTYRVVSVLTVLHQDWL NGKEYKCKVSNKALPAPIEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 167); and
      a light chain (LC) having the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO: 166); or
   (7) a heavy chain (HC) having the amino acid sequence QVQLVESGGGVVQPGRSLRLSCAASGFTFSNYRMHWVRQAPGKGLEWIAVIKVKSDNYGANYA DSVKGRFTISRDNSKNTLYLQMNSLRAEDTAVYYCSSPTYPGSSGFAYWGQGTLVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPSVFLFPPKPKDTLM ISRTPEVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 168); and
      a light chain (LC) having the amino acid sequence DIQMTQSPSSLSASVGDRVTITCRASQDISNYLNWYQQKPDGTVKLLIYYTSRLHTGVPSRFSGS GSGTDYTLTISNLQQEDIATYFCQQGNKFPPTFGGGTKVEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 169); or
   (8) a heavy chain (HC) having the amino acid sequence EVQLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPSVFLF PPKPKDTLMISRTPEVTCVVVDVSHEDPEVKFNWYVDGVEVHNAKTKPREEQYNSTYRVVSVLT VLHQDWLNGKEYKCKVSNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 170); and
      a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
   (9) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPAPEEKTISKAKGQPREPQVYTLPPSRDELTKNQVSLTCLVKG FYPSDIAVEWESNGQPENNYKTTPPVLDSDGSFFLYSKLTVDKSRWQQGNVFSCSVMHEALHN HYTQKSLSLSPG (SEQ ID NO: 172); and
      a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
   (10) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPLPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 173); and
      a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
   (11) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSWVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNKALPAPIEKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 174); and
      a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
   (12) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNWALPAPISKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 175); and
      a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
   (13) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPSVFLFPPKPKDTLM ISRTPEVTCVWVDVSHEDPEVKFNWYVDGVEVHNAKTKPREEQYNSTYRVVSVLTVLHQDWLN GKEYKCKVSNKALPAPIEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 176); and
      a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
   (14) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPDVFLFPPKPKDTLM ISRTPEVTCVVVDVSHEDPEVKFNWYVDGVEVHNAKTKPREEQYNSTYRVVSVLTVLHQDWLN GKEYKCKVSNKALPAPEEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 178); and
      a light chain (LC) having the amino acid sequence DWVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
   (15) a heavy chain (HC) having the acid sequence amino EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPDVFLFPPKPKDTLM ISRTPEVTCVVVDVSHEDPEVKFNWYVDGVEVHNAKTKPREEQYNSTYRVVSVLTVLHQDWLN GKEYKCKVSNKALPLPEEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 179); and
      a light chain (LC) having the amino acid sequence DWVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
   (16) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELVGGPSVFLLPPKPKDTLM ISRTPEVTCVVVDVSHEDPEVKFNWYVDGVEVHNAKTKPPEEQYNSTLRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPLVLDSDGSFFLYSKLTVDKSRWQQGNVFSCSVMHEALHNHYTQKSLSLSP G (SEQ ID NO: 180); and
      a light chain (LC) having the amino acid sequence DWVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASWVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
   (17) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELVGGPSVFLLPPKPKDTLM ISRTPEVTCVVVDVSHEDPEVKFNWYVDGVEVHNAKTKPPEEQYNSTLRVVSVLTVLHQDWLNG KEYKCKVSNWALPAPISKTISKAKGQPREPQVYTLPPSRDELTKNQVSLTCLVKGFYPSDIAVEW ESNGQPENNYKTTPLVLDSDGSFFLYSKLTVDKSRWQQGNVFSCSVMHEALHNHYTQKSLSLS PG (SEQ ID NO: 181); and
      a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
   (18) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVTVPSSSLGTQTYICNVNHKPSNTKVDKKVEPKSCDKTHTCPPCPAPELLGGPSVFLF PPKPKDTLMISRTPEVTCVVVDVSHEDPEVKFNWYVDGVEVHNAKTKPREEQYNSTYRVVSVLT VLHQDWLNGKEYKCKVSNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 182); and
      a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
   (19) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSWVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPAPEEKTISKAKGQPREPQVYTLPPSRDELTKNQVSLTCLVKG FYPSDIAVEWESNGQPENNYKTTPPVLDSDGSFFLYSKLTVDKSRWQQGNVFSCSVMHEALHN HYTQKSLSLSPG (SEQ ID NO: 184); and
      a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
   (20) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPLPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 185); and
      a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
   (21) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSWVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNKALPAPIEKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 186); and
      a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
   (22) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNWALPAPISKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 187); and
      a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
   (23) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPSVFLFPPKPKDTLMI SRTPEVTCVVVDVSHEDPEVKFNWYVDGVEVHNAKTKPREEQYNSTYRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPPVLDSDGSFFLYSKLTVDKSRWQQGNVFSCSVMHEALHNHYTQKSLSLSP G (SEQ ID NO: 188); and
      a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
   (24) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPDVFLFPPKPKDTLMI SRTPEVTCVVVDVSHEDPEVKFNWYVDGVEVHNAKTKPREEQYNSTYRVVSVLTVLHQDWLNG KEYKCKVSNKALPAPEEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 190); and
      a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
   (25) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPDVFLFPPKPKDTLMI SRTPEVTCVVVDVSHEDPEVKFNWYVDGVEVHNAKTKPREEQYNSTYRVVSVLTVLHQDWLNG KEYKCKVSNKALPLPEEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 191); and
      a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
   (26) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELVGGPSVFLLPPKPKDTLMI SRTPEVTCVVVDVSHEDPEVKFNWYVDGVEVHNAKTKPPEEQYNSTLRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPLVLDSDGSFFLYSKLTVDKSRWQQGNVFSCSVMHEALHNHYTQKSLSLSP G (SEQ ID NO: 192); and
      a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
   (27) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELVGGPSVFLLPPKPKDTLMI SRTPEVTCVVVDVSHEDPEVKFNWYVDGVEVHNAKTKPPEEQYNSTLRVVSVLTVLHQDWLNG KEYKCKVSNWALPAPISKTISKAKGQPREPQVYTLPPSRDELTKNQVSLTCLVKGFYPSDIAVEW ESNGQPENNYKTTPLVLDSDGSFFLYSKLTVDKSRWQQGNVFSCSVMHEALHNHYTQKSLSLS PG (SEQ ID NO: 193); and
      a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189).
14. A monoclonal antibody or an antigen-binding fragment thereof, wherein said antibody or antigen-binding fragment specifically binds to human CCR8, and wherein said antibody or antigen-binding fragment comprises:
   (1) a heavy chain variable domain (VH) comprising a CDR-H1 having the amino acid sequence PYAMN (SEQ ID NO: 6), a CDR-H2 having the amino acid sequence RIRSKSNNYATYYADSVKD (SEQ ID NO: 7), and a CDR-H3 having the amino acid sequence GREAYYRYDGDYYAMDY (SEQ ID NO: 8); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
   (2) a heavy chain variable domain (VH) comprising a CDR-H1 having the amino acid sequence TYAMN (SEQ ID NO: 12), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
   (3) a heavy chain variable domain (VH) comprising a CDR-H1 having the amino acid sequence EYTIH (SEQ ID NO: 14), a CDR-H2 having the amino acid sequence GINPNGDTRYDQKFKG (SEQ ID NO: 15), and a CDR-H3 having the amino acid sequence VARFYGISPYAMDY (SEQ ID NO: 16); and
      a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQSTHVPYT (SEQ ID NO: 19); or
   (4) a heavy chain variable domain (VH) comprising a CDR-H1 having the amino acid sequence AYAMN (SEQ ID NO: 20), a CDR-H2 having the amino acid sequence RIRSKSNDYATYYGDSVKD (SEQ ID NO: 21), and a CDR-H3 having the amino acid sequence GREAYYRYDGGYYAMDY (SEQ ID NO: 13); and
      a light chain variable domain (VL) comprising a CDR-L1 having the amino acid sequence TSSKSLLHSNGNTYLY (SEQ ID NO: 22), a CDR-L2 having the amino acid sequence RMSNLAS (SEQ ID NO: 10), and a CDR-L3 having the amino acid sequence MQHREYPFT (SEQ ID NO: 23); or
   (5) a heavy chain variable domain (VH) comprising a CDR-H1 having the amino acid sequence DYVVT (SEQ ID NO: 24), a CDR-H2 having the amino acid sequence VIWGGGNTYYNSDLKS (SEQ ID NO: 25), and a CDR-H3 having the amino acid sequence RHRDYALDY (SEQ ID NO: 26); and
      a light chain variable domain (VL) comprising a CDR-L1 having the amino acid sequence RSSQSLVYSNGNTYLH (SEQ ID NO: 27), a CDR-L2 having the amino acid sequence KVSNRFS (SEQ ID NO: 18), and a CDR-L3 having the amino acid sequence SQSTYVPPT (SEQ ID NO: 28); or
   (6) a heavy chain variable domain (VH) comprising a CDR-H1 having the amino acid sequence DYTLH (SEQ ID NO: 29), a CDR-H2 having the amino acid sequence GITPKNGDTRYDPRFKD (SEQ ID NO: 30), and a CDR-H3 having the amino acid sequence VARFYGVSPYAMDY (SEQ ID NO: 31); and
      a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQTTHVPYT (SEQ ID NO: 32); or
   (7) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PDGFSPFVY (SEQ ID NO: 35); and
      a light chain variable domain (VL) comprising a CDR-L1 having the amino acid sequence KASQDINSYLS (SEQ ID NO: 36), a CDR-L2 having the amino acid sequence RANRLVD (SEQ ID NO: 37), and a CDR-L3 having the amino acid sequence LQYGEFPPT (SEQ ID NO: 38); or
   (8) a heavy chain variable domain (VH) comprising a CDR-H1 having the amino acid sequence AYAMN (SEQ ID NO: 20), a CDR-H2 having the amino acid sequence RIRSKSNNYATYYGDSVKD (SEQ ID NO: 39), and a CDR-H3 having the amino acid sequence GREAYYRYDGDYYAMDY (SEQ ID NO: 8); and
      a light chain variable domain (VL) comprising a CDR-L1 having the amino acid sequence KSSQSLLNSSNQKNYLA (SEQ ID NO: 40), a CDR-L2 having the amino acid sequence FASTRES (SEQ ID NO: 41), and a CDR-L3 having the amino acid sequence QQHYSTPYT (SEQ ID NO: 42); or
   (9) a heavy chain variable domain (VH) comprising a CDR-H1 having the amino acid sequence AYAMN (SEQ ID NO: 20), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
   (10) a heavy chain variable domain (VH) comprising a CDR-H1 having the amino acid sequence EYTIH (SEQ ID NO: 14), a CDR-H2 having the amino acid sequence GINPNNGNTRYDQKFKG (SEQ ID NO: 43), and a CDR-H3 having the amino acid sequence VARSSGSGPYAMDY (SEQ ID NO: 44); and
      a light chain variable domain (VL) comprising a CDR-L1 having the amino acid sequence RCTQSLLHTNGDTYLH (SEQ ID NO: 45), a CDR-L2 having the amino acid sequence KVSNRFS (SEQ ID NO: 18), and a CDR-L3 having the amino acid sequence SQSTHVPYT (SEQ ID NO: 19); or
   (11) a heavy chain variable domain (VH) comprising a CDR-H1 having the amino acid sequence GYNMH (SEQ ID NO: 46), a CDR-H2 having the amino acid sequence AISPGKGDTSYNLKFKG (SEQ ID NO: 47), and a CDR-H3 having the amino acid sequence SGGTPFAY (SEQ ID NO: 48); and
      a light chain variable domain (VL) comprising a CDR-L1 having the amino acid sequence RSSQSLVHSNGDTYLY (SEQ ID NO: 49), a CDR-L2 having the amino acid sequence KVSNRFS (SEQ ID NO: 18), and a CDR-L3 having the amino acid sequence SQSTHVPYT (SEQ ID NO: 19); or
   (12) a heavy chain variable domain (VH) comprising a CDR-H1 having the amino acid sequence ANAMN (SEQ ID NO: 50), a CDR-H2 having the amino acid sequence RIRSKSNNYATYYADSVKD (SEQ ID NO: 7), and a CDR-H3 having the amino acid sequence GSDNYIFYAMDY (SEQ ID NO: 51); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
   (13) a heavy chain variable domain (VH) comprising a CDR-H1 having the amino acid sequence TNAMN (SEQ ID NO: 52), a CDR-H2 having the amino acid sequence RIRSKSNYYATYYADSVKD (SEQ ID NO: 53), and a CDR-H3 having the amino acid sequence GREMGNYYSMDY (SEQ ID NO: 54); and
      a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
   (14) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PTYPGSSGFAY (SEQ ID NO: 55); and
      a light chain variable domain (VL) comprising a CDR-L1 having the amino acid sequence RASQDISNYLN (SEQ ID NO: 56), a CDR-L2 having the amino acid sequence YTSRLHS (SEQ ID NO: 57), and a CDR-L3 having the amino acid sequence QQGNKFPPT (SEQ ID NO: 58); or
   (15) a heavy chain variable domain (VH) comprising a CDR-H1 having the amino acid sequence EYTMH (SEQ ID NO: 59), a CDR-H2 having the amino acid sequence GINPNNGDTNYNQKFMG (SEQ ID NO: 60), and a CDR-H3 having the amino acid sequence RLLRRGAMDY (SEQ ID NO: 61); and
      a light chain variable domain (VL) comprising a CDR-L1 having the amino acid sequence RASENVEYYGTSLMQ (SEQ ID NO: 62), a CDR-L2 having the amino acid sequence AASNVDS (SEQ ID NO: 63), and a CDR-L3 having the amino acid sequence QQSRKVPWT (SEQ ID NO: 64); or
   (16) a heavy chain variable domain (VH) comprising a CDR-H1 having the amino acid sequence GSYMH (SEQ ID NO: 65), a CDR-H2 having the amino acid sequence RINPYNGATSYNQNFKD (SEQ ID NO: 66), and a CDR-H3 having the amino acid sequence TLLRLLDY (SEQ ID NO: 67); and
      a light chain variable domain (VL) comprising a CDR-L1 having the amino acid sequence RSSKSLLHSNGITYLY (SEQ ID NO: 68), a CDR-L2 having the amino acid sequence QMSNLAS (SEQ ID NO: 69), and a CDR-L3 having the amino acid sequence AQNLELPWT (SEQ ID NO: 70); or
   (17) a heavy chain variable domain (VH) comprising a CDR-H1 having the amino acid sequence SYWMH (SEQ ID NO: 71), a CDR-H2 having the amino acid sequence NIWPGSASTNYDEKFKN (SEQ ID NO: 72), and a CDR-H3 having the amino acid sequence GGKGAMDY (SEQ ID NO: 73); and
      a light chain variable domain (VL) comprising a CDR-L1 having the amino acid sequence RSSQSLENSYGNTYLN (SEQ ID NO: 74), a CDR-L2 having the amino acid sequence RVSNRFS (SEQ ID NO: 75), and a CDR-L3 having the amino acid sequence LQVTHVPPT (SEQ ID NO: 76); or
   (18) a heavy chain variable domain (VH) comprising a CDR-H1 having the amino acid sequence TYAMH (SEQ ID NO: 77), a CDR-H2 having the amino acid sequence HINPSSGYSNYNQKFKD (SEQ ID NO: 78), and a CDR-H3 having the amino acid sequence SEVRRGYFDV (SEQ ID NO: 79); and
      a light chain variable domain (VL) comprising a CDR-L1 having the amino acid sequence RTSQDIRNYLN (SEQ ID NO: 80), a CDR-L2 having the amino acid sequence YTSRLHS (SEQ ID NO: 57), and a CDR-L3 having the amino acid sequence QQGNTLPPT (SEQ ID NO: 81); or
   (19) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDV (SEQ ID NO: 124); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11); or
   (20) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23); or
   (21) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
      a light chain variable domain (VL) comprising a CDR-L1 having the amino acid sequence KSLLHSNGNTY (SEQ ID NO: 125), a CDR-L2 having the amino acid sequence RVS, and a CDR-L3 having the amino acid sequence MQHREYPFT (SEQ ID NO: 23); or
   (22) a heavy chain variable domain (VH) comprising a CDR-H1 having the amino acid sequence GYTFTEYT (SEQ ID NO: 128), a CDR-H2 having the amino acid sequence INPNNGNT (SEQ ID NO: 129), and a CDR-H3 having the amino acid sequence ARVARSSGSGPYAMDY (SEQ ID NO: 130); and
      a light chain variable domain (VL) comprising a CDR-L1 having the amino acid sequence QSLLHTNGDTY (SEQ ID NO: 131), a CDR-L2 having the amino acid sequence KVS, and a CDR-L3 having the amino acid sequence SQSTHVPYT (SEQ ID NO: 19); or
   (23) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDY (SEQ ID NO: 133); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23); or
   (24) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDY (SEQ ID NO: 133); and
      a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11); or
   (25) a heavy chain variable domain (VH) comprising a CDR-H1 having the amino acid sequence GFTFSNYR (SEQ ID NO: 134), a CDR-H2 having the amino acid sequence IKVKSDNYGA (SEQ ID NO: 135), and a CDR-H3 having the amino acid sequence SSPTYPGSSGFAY (SEQ ID NO: 136); and
      a light chain variable domain (VL) comprising a CDR-L1 having the amino acid sequence QDISNY (SEQ ID NO: 137), a CDR-L2 having the amino acid sequence YTS, and a CDR-L3 having the amino acid sequence QQGNKFPPT (SEQ ID NO: 58).
15. A monoclonal antibody or an antigen-binding fragment thereof, wherein said antibody or antigen-binding fragment specifically binds to human CCR8, and wherein said antibody or antigen-binding fragment comprises:
   (1) a heavy chain variable domain (VH) having an amino acid sequence with at least 80% sequence identity to the amino acid sequence MLLGLKWVFFVVFYQGVLCEVQLLESGGGLVQPKGSLKLSCAASGFTFNPYAMNWVRQAPGRG LEWVARIRSKSNNYATYYADSVKDRFTISRDDSQDMLYLQMNNLKTEDTAMYYCVRGREAYYRY DGDYYAMDYWGQGTSVTVSS (SEQ ID NO: 82); and
      a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
   (2) a heavy chain variable domain (VH) having an amino acid sequence with at least 80% sequence identity to the amino acid sequence MLLGLKWVFFVVFYQGVHCEVQLVESGGGLVQPKGSLKLSCAASGFTFNTYAMNWVRQAPGK GLEWVARIRSKSNNYATYYADSVKDRFTISRDDSQSMLYLQMNNLKTEDTAMYYCVRGREAYYR YDGGYYAMDYWGQGTSVTVSS (SEQ ID NO: 84); and
      a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
   (3) a heavy chain variable domain (VH) having an amino acid sequence with at least 80% sequence identity to the acid sequence amino MGWSWIFLFLLSGTAGVLSKVQLQQSGPELVKPGASVKISCKTSGYTFTEYTIHWVQQIHGKSPE WIGGINPNGDTRYDQKFKGKATLTIDKSSSTAYMELRSLTSEDSAVYYCARVARFYGISPYAMDY WGQGTSVTVSS (SEQ ID NO: 85); and
      a light chain variable domain (VL) having an amino acid sequence with at least 80% sequence identity to the amino acid sequence MKLPVRLLVLMFWIPASSSDVVMTQTPLSLPVSLGDQASISCRCTQSLLHSNGDTYLHWYLQKP GQSPNLLIYKVSNRFSGVPDRFSGSGSGTDFTLKINRVEAEDLGVYFCSQSTHVPYTFGGGTNLE IK (SEQ ID NO: 86); or
   (4) a heavy chain variable domain (VH) having an amino acid sequence with at least 80% sequence identity to the amino acid sequence MLLGLKWVFFVVFYQGVHCEVQLVESGGGLVQPTGSLRLSCAASGFTFNAYAMNWVRQAPGK GLEWVARIRSKSNDYATYYGDSVKDRFTISRDDSQSMLYLQMNNLKTEDTAMYYCVRGREAYY RYDGGYYAMDYWGQGTSVTVSS (SEQ ID NO: 87); and
      a light chain variable domain (VL) having an amino acid sequence with at least 80% sequence identity to the amino acid sequence MRCLAEFLGLLVLWIPGAIGDIVMTQAAPSVSVTPGESVSISCTSSKSLLHSNGNTYLYWFLQRPG QSPQLLIYRMSNLASGVPDRFSGSGSGTAFTLRISRVEAEDVGVYYCMQHREYPFTFGSGTKLEI K (SEQ ID NO: 88); or
   (5) a heavy chain variable domain (VH) having an amino acid sequence with at least 80% sequence identity to the amino acid sequence MAVLGLLLCLVTFPSCVLSQVQLKESGPGLVAPSQSLSITCTVSGLSMNDYVVTWIRQPPGKGLE WLGVIWGGGNTYYNSDLKSRLSITKDNSKSQVFFKMSSLQTDDTAVYYCARRHRDYALDYWGQ GISVTVSS (SEQ ID NO: 89); and
      a light chain variable domain (VL) having an amino acid sequence with at least 80% sequence identity to the amino acid sequence MKLPVRLLVLMFWIPASSSDVVMTQTPLSLPVSLGDQASISCRSSQSLVYSNGNTYLHWYLQKP GQSPKLLIYKVSNRFSGVPDRFSGSGSGTDFTLKISRVEAEDLGVYFCSQSTYVPPTFGGGTKLEI K (SEQ ID NO: 90); or
   (6) a heavy chain variable domain (VH) having an amino acid sequence with at least 80% sequence identity to the amino acid sequence MGWSWIFLFLLSGTAGVLSEVQLQQSGPELLKPGTSVKISCTTSGYTFSDYTLHWVKQSHGKSL EWIGGITPKNGDTRYDPRFKDKATLTIDKSSSAAYMELRSLTSEDSAVYYCARVARFYGVSPYAM DYWGQGASVTVSS (SEQ ID NO: 91); and
      a light chain variable domain (VL) having an amino acid sequence with at least 80% sequence identity to the amino acid sequence MKLPVRLLVLMFWIPVSNSDVVMTQTPLSLPVSLGDQASISCRCTQSLLHSNGDTYLHWYLQKP GQSPKLLIYKVSNRFSGVPDRFSGSGSGTDFTLEITRVEAEDLGVYFCSQTTHVPYTFGGGTKLEI K (SEQ ID NO: 92); or
(7) a heavy chain variable domain (VH) having an amino acid sequence with at least 80% sequence identity to the amino acid sequence MELGLSWVFLVALLNGVQCQVHLVETGGGLVRPGNSLKLSCVTSGFTLSNYRMHWLRQPPGKR LEWIAVIKVKSDNYGANYAESVKGRFTISRDDAKSSVYLQMNRLREEDTATYYCSRPDGFSPFVY WGQGTLVTVSA (SEQ ID NO: 93); and
   a light chain variable domain (VL) having an amino acid sequence with at least 80% sequence identity to the amino acid sequence MDMRTPAQFLGILLLWFPGIKCDIKMTQSPSSMYASLGERVTITCKASQDINSYLSWFQQKPGKS PKTLIYRANRLVDGVPSRFSGSGSGQDYFLTISSLEYEDMGIYYCLQYGEFPPTFGAGTKLELK (SEQ ID NO: 94); or
(8) a heavy chain variable domain (VH) having an amino acid sequence with at least 80% sequence identity to amino acid the sequence MLLGLKWVFFVVFYQGVHCEVQLVESGGGLVQPKGSLRLSCAASGFTFNAYAMNWVRQAPGK GLEWVARIRSKSNNYATYYGDSVKDRFTISRDDSQTMLYLQMNNLKTEDTAMYYCVRGREAYYR YDGDYYAMDYWGQGTSVTVSS (SEQ ID NO: 95); and
   a light chain variable domain (VL) having an amino acid sequence with at least 80% sequence identity to the amino acid sequence MESQTQVLMFLLLWVSGACADIVMTQSPSSLAMSVGQKVTMSCKSSQSLLNSSNQKNYLAWYQ QKPGQSPKLLVYFASTRESGVPDRFIGSGSGTDFTLTISSVQAEDLADYFCQQHYSTPYTFGGGT KLEIK (SEQ ID NO: 96); or
(9) a heavy chain variable domain (VH) having an amino acid sequence with at least 80% sequence identity to the amino acid sequence MLLGLKWVFFWVFYQGVHCEVQLVESGGGLVQPKGSLKLSCAASGFTFNAYAMNWVRQAPGK GLEWLARIRSKSNNYATYYADSVKDRFTISRDDSQSMFYLQMNNLKSEDTAMYYCVRGREAYYR YDGGYYAMDYWGQGTSVTVSS (SEQ ID NO: 97); and
   a light chain variable domain (VL) having an amino acid sequence with at least 80% sequence identity to the amino acid sequence MRCLAEFLGLLVLWIPGAIGEIVMTQAALSAPVTPGESVSMSCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 98); or
(10) a heavy chain variable domain (VH) having an amino acid sequence with at least 80% sequence identity to the amino acid sequence MGWSWIFLFLLSGTAGVLSEVQLQQSGPELVKPGASVKISCKTSGYTFTEYTIHWVKQSHGESLE WVGGINPNNGNTRYDQKFKGKATLTIDKSSSPAYMELRSLTSEDSAVYYCARVARSSGSGPYAM DYWGQGTSVTVSS (SEQ ID NO: 99); and
   a light chain variable domain (VL) having an amino acid sequence with at least 80% sequence identity to the amino acid sequence MKLPVRLLVLMFWIPVSSSDVVMTQTPLSLPVSLGDQASISCRCTQSLLHTNGDTYLHWYLQKPG QSPKLLIYKVSNRFSGVPDRFSGSGSGTDFTLKISRVEAEDLGVYFCSQSTHVPYTFGGGTKLEIK (SEQ ID NO: 100); or
(11) a heavy chain variable domain (VH) having an amino acid sequence with at least 80% sequence identity to the amino acid sequence MGWSCIILFLVATATDVHSQVQLQQPGAELVKPGASVKMSCKATGYTFTGYNMHWVKQTPGQG LEWIGAISPGKGDTSYNLKFKGKATLTTDKSSSTAYMQLSSLTSADSAVYYCARSGGTPFAYWG QGTLVTVSA (SEQ ID NO: 101); and
   a light chain variable domain (VL) having an amino acid sequence with at least 80% sequence identity to the amino acid sequence MKLPVRLLVLMFWIPASSSDVVMTQTPLSLPVSLGDQASISCRSSQSLVHSNGDTYLYWYLQKP GQSPKLLIYKVSNRFSGVPDRFSGSGSGTDFTLKINRVEAEDLGVYFCSQSTHVPYTFGGGTKLE IK (SEQ ID NO: 102); or
(12) a heavy chain variable domain (VH) having an amino acid sequence with at least 80% sequence identity to the amino acid sequence MLLGLKWVFFVVFYQGVHCEVQLVETGGGLVQPKGSLKLSCAASGFTFNANAMNWVRQAPGK GLEWVARIRSKSNNYATYYADSVKDRFTISRDDSQSMLYLQMNNLKTEDTAMYYCVRGSDNYIF YAMDYWGQGTSVTVSS (SEQ ID NO: 103); and
   a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGGGTKL EIK (SEQ ID NO: 104); or
(13) a heavy chain variable domain (VH) having an amino acid sequence with at least 80% sequence identity to the amino acid sequence MLLGLKWVFFVVFYQGVHCEVQLVETGGGLVQPKGSLKLSCAASGFTFNTNAMNWVRQAPGK GLEWVARIRSKSNYYATYYADSVKDRFTISRDDSQSMLYLQMNNLKTEDTAMYYCVRGREMGN YYSMDYWGQGTSVTVSS (SEQ ID NO: 105); and
   a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
(14) a heavy chain variable domain (VH) having an amino acid sequence with at least 80% sequence identity to the amino acid sequence MELGLSWVFLVALLNGVQCQVQLVETGGGLVRPGNSLKLSCVTSGFTFSNYRMHWLRQPPGKR LEWIAVIKVKSDNYGANYAESVKGRFTISRDDSKSSVYLQVNRLREEDTATYYCSSPTYPGSSGF AYWGQGTLVTVSA (SEQ ID NO: 106); and
   a light chain variable domain (VL) having an amino acid sequence with at least 80% sequence identity to the amino acid sequence MMSSAQFLGLLLLCFQGTRCDIQMTQTTSSLSASLGDRVTISCRASQDISNYLNWYQQKPDGTV KLLIYYTSRLHSGVPSRFSGSGSGTDYSLTISNLEQEDIATYFCQQGNKFPPTFGGGTKLEIN (SEQ ID NO: 107); or
(15) a heavy chain variable domain (VH) having an amino acid sequence with at least 80% sequence identity to the amino acid sequence MGWSWIFLFLLSGTAGVLSEVQLQQSGPELVKPGASVKISCKTSGYTFTEYTMHWVKQSHGKSL EWIGGINPNNGDTNYNQKFMGKATLTVDKSSSTAYMELRSLTSEDSAVYYCVRRLLRRGAMDY WGQGTSVTVSS (SEQ ID NO: 108); and
   a light chain variable domain (VL) having an amino acid sequence with at least 80% sequence identity to the amino acid sequence MESDTLLLWVLLLWVPGSTGDIVLTQSPASLAVSLGQRATISCRASENVEYYGTSLMQWFQQKP GQPPKLLIYAASNVDSGVPARFSGSGSGTDFSLNIHPVEEDDIAMYFCQQSRKVPWTFGGGTKL DIK (SEQ ID NO: 109); or
(16) a heavy chain variable domain (VH) having an amino acid sequence with at least 80% sequence identity to the amino acid sequence MGWSWIFLFLLSGTAGVLSEVQLQQSGPELVKPGASVKISCKASVYSFTGSYMHWVKQSHVKSL EWIGRINPYNGATSYNQNFKDKASLTVDKSSSTAYMELHSLTSEDSAVYYCATTLLRLLDYWGQ GTTLTVSS (SEQ ID NO: 110); and
   a light chain variable domain (VL) having an amino acid sequence with at least 80% sequence identity to the amino acid sequence MRFSAQLLGLLVLWIPGSTADIVMTQAAFSNPVTLGTSASISCRSSKSLLHSNGITYLYWYLQKPG QSPQLLIYQMSNLASGVPDRESSSGSGTDFTLRISRVEAEDVGVYYCAQNLELPWTFGGGTKLEI K (SEQ ID NO: 111); or
(17) a heavy chain variable domain (VH) having an amino acid sequence with at least 80% sequence identity to the amino acid sequence MGWSSIILFLVATASGVHSQVQLQQPGSELVRPGASVKLSCKASGYTFTSYWMHWVKQRPGQG LEWIGNIWPGSASTNYDEKFKNKATLTVDTSSSTAYMQLSSLTSEDSAVYYCIRGGKGAMDYWS QGTSVTVSS (SEQ ID NO: 112); and
   a light chain variable domain (VL) having an amino acid sequence with at least 80% sequence identity to the amino acid sequence MKLPVRLLVLMFWIPVSSSDVVMTQIPLSLPVSLGDQASISCRSSQSLENSYGNTYLNWYLQKPG QSPQLLIYRVSNRFSGVLDRFSGSGSGTDFTLKISRVEAEDLGVYFCLQVTHVPPTFGAGTKLEL K (SEQ ID NO: 113); or
(18) a heavy chain variable domain (VH) having an amino acid sequence with at least 80% sequence identity to the amino acid sequence MERHWIFLLLLSVTAGVHSQVQVQQSGAELARPGASVKMSCKASGYTFTTYAMHWVKQRPGQ GLEWIGHINPSSGYSNYNQKFKDKATLTADKSSSTAYMQLSSLTSEDSAVYYCARSEVRRGYFD VWGAGTTVTVSS (SEQ ID NO: 114); and
   a light chain variable domain (VL) having an amino acid sequence with at least 80% sequence identity to the amino acid sequence MMSSAQFLGLLLLCFQGTRCDIQMTQTTSSLSASLGDRVTISCRTSQDIRNYLNWYQQKPDGTV KLLISYTSRLHSGVPSRFSGSGSGTDYSLTISNLEQEDFATYFCQQGNTLPPTFGGGTRVEIK (SEQ ID NO: 115); or
(19) a heavy chain variable domain (VH) having an amino acid sequence with at least 80% sequence identity to the amino acid sequence EVOLVESGGGLVQPGRSLRLSCTASGFTFNTYAMNWVRQAPGKGLEWVARIRSKSNNYATYYA ASVKGRFTISRDDSKSIAYLQMNSLKTEDTAVYYCVRGREAYYRYDGGYYAMDVWGQGTTVTV SS (SEQ ID NO: 138); and
   a light chain variable domain (VL) having an amino acid sequence with at least 80% sequence identity to the amino acid sequence DIVMTQAAPSLPVTPGESASISCRSSKSLLHSNGNTYLYWFLQKPGQSPQLLIYRMSNLASGVPD RFSGSGSGTAFTLKISRVEAEDVGVYYCMQHLEYPFTFGSGTKLEIK (SEQ ID NO: 139); or
(20) a heavy chain variable domain (VH) having an amino acid sequence with at least 80% sequence identity to the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SS (SEQ ID NO: 140); and
   a light chain variable domain (VL) having an amino acid sequence with at least 80% sequence identity to the amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIK (SEQ ID NO: 141); or
(21) a heavy chain variable domain (VH) having an amino acid sequence with at least 80% sequence identity to the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SS (SEQ ID NO: 140); and
   a light chain variable domain (VL) having an amino acid sequence with at least 80% sequence identity to the amino acid sequence DIVMTQSPLSLPVTPGEPASISCRSSKSLLHSNGNTYLYWFLQKPGQSPQLLIYRVSNLASGVPD RFSGSGSGTDFTLKISRVEAEDVGVYYCMQHREYPFTFGQGTKLEIK (SEQ ID NO: 142); or
(22) a heavy chain variable domain (VH) having an amino acid sequence with at least 80% sequence identity to the amino acid sequence QVQLVQSGPEVVKPGASVKVSCKTSGYTFTEYTIHWVRQAPGQSLEWVGGINPNNGNTRYDQK FQGRVTITRDKSASTAYMELRSLTSEDTAVYYCARVARSSGSGPYAMDYWGQGTTVTVSS (SEQ ID NO: 143); and
   a light chain variable domain (VL) having an amino acid sequence with at least 80% sequence identity to the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIK (SEQ ID NO: 144); or
(23) a heavy chain variable domain (VH) having an amino acid sequence with at least 80% sequence identity to the amino acid sequence QVQLQQSGPEVVKPGASVKVSCKTSGYTFTEYTIHWVRQSHGQSLEWVGGINPNNGNTRYDQK FKGRVTITIDKSSSTAYMELRSLTSEDTAVYYCARVARSSGSGPYAMDYWGQGTTVTVSS (SEQ ID NO: 145); and
   a light chain variable domain (VL) having an amino acid sequence with at least 80% sequence identity to the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIK (SEQ ID NO: 144); or
(24) a heavy chain variable domain (VH) having an amino acid sequence with at least 80% sequence identity to the amino acid sequence QVQLVESGGGVVQPGRSLRLSCAASGFTFSNYRMHWVRQAPGKGLEWIAVIKVKSDNYGANYA DSVKGRFTISRDNSKNTLYLQMNSLRAEDTAVYYCSSPTYPGSSGFAYWGQGTLVTVSS (SEQ ID NO: 146); and
   a light chain variable domain (VL) having an amino acid sequence with at least 80% sequence identity to the amino acid sequence DIQMTQSPSSLSASVGDRVTITCRASQDISNYLNWYQQKPDGTVKLLIYYTSRLHTGVPSRFSGS GSGTDYTLTISNLQQEDIATYFCQQGNKFPPTFGGGTKVEIN (SEQ ID NO: 132); or
(25) a heavy chain variable domain (VH) having an amino acid sequence with at least 80% sequence identity to the amino acid sequence EVQLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSS (SEQ ID NO: 147); and
   a light chain variable domain (VL) having an amino acid sequence with at least 80% sequence identity to the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIK (SEQ ID NO: 148); or
(26) a heavy chain variable domain (VH) having an amino acid sequence with at least 80% sequence identity to the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSS (SEQ ID NO: 149); and
   a light chain variable domain (VL) having an amino acid sequence with at least 80% sequence identity to the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIK (SEQ ID NO: 150); or
(27) a heavy chain variable domain (VH) having an amino acid sequence with at least 80% sequence identity to the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSS (SEQ ID NO: 151); and
   a light chain variable domain (VL) having an amino acid sequence with at least 80% sequence identity to the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIK (SEQ ID NO: 152); or
(28) a heavy chain variable domain (VH) having an amino acid sequence with at least 80% sequence identity to the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSA (SEQ ID NO: 153); and
   a light chain variable domain (VL) having an amino acid sequence with at least 80% sequence identity to the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEIN (SEQ ID NO: 154).
16. A monoclonal antibody or an antigen-binding fragment thereof, wherein said antibody or antigen-binding fragment specifically binds to human CCR8, and wherein said antibody or antigen-binding fragment comprises:
   (1) a heavy chain (HC) having the amino acid sequence EVOLVESGGGLVQPGRSLRLSCTASGFTFNTYAMNWVRQAPGKGLEWVARIRSKSNNYATYYA ASVKGRFTISRDDSKSIAYLQMNSLKTEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSWVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 159); and
      a light chain (LC) having the amino acid sequence DIVMTQAAPSLPVTPGESASISCRSSKSLLHSNGNTYLYWFLQKPGQSPQLLIYRMSNLASGVPD RFSGSGSGTAFTLKISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 160); or
   (2) a heavy chain (HC) having the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 161); and
   a light chain (LC) having the amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 162); or
(3) a heavy chain (HC) having the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSWVTVPSSSLGTQTYICNVNHKPSNTKVDKKVEPKSCDKTHTCPPCPAPELLGGPDVFLFP PKPKDTLMISRTPEVTCVVVDVSHEDPEVKFNWYVDGVEVHNAKTKPREEQYNSTYRVVSVLTV LHQDWLNGKEYKCKVSNKALPAPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 163); and
   a light chain (LC) having the amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 162); or
(4) a heavy chain the amino acid sequence (HC) having EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 161); and
   a light chain (LC) having the amino acid sequence DIVMTQSPLSLPVTPGEPASISCRSSKSLLHSNGNTYLYWFLQKPGQSPQLLIYRVSNLASGVPD RFSGSGSGTDFTLKISRVEAEDVGVYYCMQHREYPFTFGQGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 164); or
(5) a heavy chain (HC) having the amino acid sequence QVQLVQSGPEVVKPGASVKVSCKTSGYTFTEYTIHWVRQAPGQSLEWVGGINPNNGNTRYDQK FQGRVTITRDKSASTAYMELRSLTSEDTAVYYCARVARSSGSGPYAMDYWGQGTTVTVSSASTK GPSVFPLAPSSKSTSGGTAALGCLVKDYFPEPVTVSWNSGALTSGVHTFPAVLQSSGLYSLSSV VTVPSSSLGTQTYICNVNHKPSNTKVDKKVEPKSCDKTHTCPPCPAPELLGGPSVFLFPPKPKDT LMISRTPEVTCVVVDVSHEDPEVKFNWYVDGVEVHNAKTKPREEQYNSTYRVVSVLTVLHQDWL NGKEYKCKVSNKALPAPIEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 165); and
   a light chain (LC) having the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO: 166); or
(6) a heavy chain (HC) having the amino acid sequence QVQLQQSGPEVVKPGASVKVSCKTSGYTFTEYTIHWVRQSHGQSLEWVGGINPNNGNTRYDQK FKGRVTITIDKSSSTAYMELRSLTSEDTAVYYCARVARSSGSGPYAMDYWGQGTTVTVSSASTK GPSVFPLAPSSKSTSGGTAALGCLVKDYFPEPVTVSWNSGALTSGVHTFPAVLQSSGLYSLSSV VTVPSSSLGTQTYICNVNHKPSNTKVDKKVEPKSCDKTHTCPPCPAPELLGGPSVFLFPPKPKDT LMISRTPEVTCVVVDVSHEDPEVKFNWYVDGVEVHNAKTKPREEQYNSTYRVVSVLTVLHQDWL NGKEYKCKVSNKALPAPIEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 167); and
   a light chain (LC) having the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO: 166); or
(7) a heavy chain (HC) having the amino acid sequence QVQLVESGGGVVQPGRSLRLSCAASGFTFSNYRMHWVRQAPGKGLEWIAVIKVKSDNYGANYA DSVKGRFTISRDNSKNTLYLQMNSLRAEDTAVYYCSSPTYPGSSGFAYWGQGTLVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPSVFLFPPKPKDTLM ISRTPEVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 168); and
   a light chain (LC) having the amino acid sequence DIQMTQSPSSLSASVGDRVTITCRASQDISNYLNWYQQKPDGTVKLLIYYTSRLHTGVPSRFSGS GSGTDYTLTISNLQQEDIATYFCQQGNKFPPTFGGGTKVEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 169); or
(8) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVTVPSSSLGTQTYICNVNHKPSNTKVDKKVEPKSCDKTHTCPPCPAPELLGGPSVFLF PPKPKDTLMISRTPEVTCVVVDVSHEDPEVKFNWYVDGVEVHNAKTKPREEQYNSTYRVVSVLT VLHQDWLNGKEYKCKVSNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 170); and
   a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(9) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPAPEEKTISKAKGQPREPQVYTLPPSRDELTKNQVSLTCLVKG FYPSDIAVEWESNGQPENNYKTTPPVLDSDGSFFLYSKLTVDKSRWQQGNVFSCSVMHEALHN HYTQKSLSLSPG (SEQ ID NO: 172); and
   a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(10) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPLPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 173); and
   a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(11) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSWVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNKALPAPIEKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 174); and
   a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(12) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNWALPAPISKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 175); and
   a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(13) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPSVFLFPPKPKDTLM ISRTPEVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 176); and
   a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(14) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPDVFLFPPKPKDTLM ISRTPEVTCVVVDVSHEDPEVKFNWYVDGVEVHNAKTKPREEQYNSTYRVVSVLTVLHQDWLN GKEYKCKVSNKALPAPEEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 178); and
   a light chain (LC) having the amino acid sequence DWVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(15) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPDVFLFPPKPKDTLM ISRTPEVTCVVDVSHEDPEVKFNWYVDGVEVHNAKTKPREEQYNSTYRVVSVLTVLHQDWLN GKEYKCKVSNKALPLPEEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 179); and
   a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(16) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELVGGPSVFLLPPKPKDTLM ISRTPEVTCVVVDVSHEDPEVKFNWYVDGVEVHNAKTKPPEEQYNSTLRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPLVLDSDGSFFLYSKLTVDKSRWQQGNVFSCSVMHEALHNHYTQKSLSLSP G (SEQ ID NO: 180); and
   a light chain (LC) having the amino acid sequence DWVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(17) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELVGGPSVFLLPPKPKDTLM ISRTPEVTCVVVDVSHEDPEVKFNWYVDGVEVHNAKTKPPEEQYNSTLRVVSVLTVLHQDWLNG KEYKCKVSNWALPAPISKTISKAKGQPREPQVYTLPPSRDELTKNQVSLTCLVKGFYPSDIAVEW ESNGQPENNYKTTPLVLDSDGSFFLYSKLTVDKSRWQQGNVFSCSVMHEALHNHYTQKSLSLS PG (SEQ ID NO: 181); and
   a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(18) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPSVFLF PPKPKDTLMISRTPEVTCVVVDVSHEDPEVKFNWYVDGVEVHNAKTKPREEQYNSTYRVVSVLT VLHQDWLNGKEYKCKVSNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 182); and
   a light chain the amino acid sequence (LC) having DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(19) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPAPEEKTISKAKGQPREPQVYTLPPSRDELTKNQVSLTCLVKG FYPSDIAVEWESNGQPENNYKTTPPVLDSDGSFFLYSKLTVDKSRWQQGNVFSCSVMHEALHN HYTQKSLSLSPG (SEQ ID NO: 184); and
   a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(20) a heavy chain the amino acid sequence (HC) having EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPLPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 185); and
   a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(21) a (HC) having the amino heavy chain acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSWVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNKALPAPIEKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 186); and
   a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(22) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNWALPAPISKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 187); and
   a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(23) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPSVFLFPPKPKDTLMI SRTPEVTCVVVDVSHEDPEVKFNWYVDGVEVHNAKTKPREEQYNSTYRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPPVLDSDGSFFLYSKLTVDKSRWQQGNVFSCSVMHEALHNHYTQKSLSLSP G (SEQ ID NO: 188); and
   a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
(24) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPDVFLFPPKPKDTLMI SRTPEVTCVVVDVSHEDPEVKFNWYVDGVEVHNAKTKPREEQYNSTYRVVSVLTVLHQDWLNG KEYKCKVSNKALPAPEEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 190); and
   a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE
(25) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPDVFLFPPKPKDTLMI SRTPEVTCVVVDVSHEDPEVKFNWYVDGVEVHNAKTKPREEQYNSTYRVVSVLTVLHQDWLNG KEYKCKVSNKALPLPEEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 191); and
   a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
(26) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELVGGPSVFLLPPKPKDTLMI SRTPEVTCVVVDVSHEDPEVKFNWYVDGVEVHNAKTKPPEEQYNSTLRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPLVLDSDGSFFLYSKLTVDKSRWQQGNVFSCSVMHEALHNHYTQKSLSLSP G (SEQ ID NO: 192); and
   a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
(27) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELVGGPSVFLLPPKPKDTLMI SRTPEVTCVVVDVSHEDPEVKFNWYVDGVEVHNAKTKPPEEQYNSTLRVVSVLTVLHQDWLNG KEYKCKVSNWALPAPISKTISKAKGQPREPQVYTLPPSRDELTKNQVSLTCLVKGFYPSDIAVEW ESNGQPENNYKTTPLVLDSDGSFFLYSKLTVDKSRWQQGNVFSCSVMHEALHNHYTQKSLSLS PG (SEQ ID NO: 193); and
   a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189).
17. The antibody or antigen-binding fragment according to any one of items 1 to 16, wherein the antibody or antigen-binding fragment comprises an Fc region, preferably an IgG1 Fc region.
18. The antibody or antigen-binding fragment according to any one of items 1 to 17, wherein the antibody or antigen-binding fragment has ADCC, CDC and/or ADCP activity.
19. The antibody or antigen-binding fragment according to any one of items 1 to 18, wherein the antibody or antigen-binding fragment comprises a hypofucosylated or an afucosylated Fc region.
20. The antibody or antigen-binding fragment according to any one of items 1 to 19, wherein the antibody or antigen-binding fragment comprises an Fc region having one or more mutations enhancing ADCC and/or CDC and/or ADCP activity.
21. The antibody or antigen-binding fragment according to any one of items 1 to 20, wherein the antibody or antigen-binding fragment has an activity of depleting CCR8-positive cells.
22. The antibody or antigen-binding fragment according to any one of items 1 to 21, wherein the antibody or antigen-binding fragment has an activity of depleting CCR8-positive immune cells.
23. The antibody or antigen-binding fragment according to any one of items 1 to 21, wherein the antibody or antigen-binding fragment has an activity of depleting CCR8-positive cancer cells.
24. The antibody or antigen-binding fragment according to any one of items 1 to 23, wherein the antibody or antigen-binding fragment has an activity of inhibiting the CCL1-induced migration of CCR8-positive cells.
25. The antibody or antigen-binding fragment according to any one of items 1 to 24, wherein the antibody or antigen-binding fragment has an activity of inhibiting the CCL1-induced activation of CCR8-positive cells.
26. The antibody or antigen-binding fragment according to any one of items 21, 24 or 25, wherein said CCR8-positive cells are CCR8-positive T cells and/or CCR8-positive macrophages, preferably wherein said CCR8-positive cells are CCR8-positive T regulatory cells and/or CCR8-positive natural killer T cells (NKT cells), more preferably wherein said CCR8-positive cells are CCR8-positive T regulatory cells.
27. The antibody or antigen-binding fragment according to any one of items 1 to 26, wherein the antibody or antigen-binding fragment does not bind to peripheral immune cells from a healthy donor.
28. A nucleic acid encoding the heavy chain and/or the light chain of the antibody or antigen-binding fragment according to any one of items 1 to 27.
29. A vector comprising the nucleic acid according to item 28.
30. A host cell comprising the nucleic acid according to item 28 or the vector according to item 29.
31. A method of producing the antibody or antigen-binding fragment according to any one of items 1 to 27, the method comprising culturing the host cell according to item 30 and isolating the antibody or antigen-binding fragment.
32. The method of item 31, wherein the host cell is a CHO-K1 cell.
33. The method of item 31 or 32, wherein the host cell is a host cell for hypofucosylation or afucosylation.
34. An antibody or antigen-binding fragment as defined in any one of items 1 to 27, which is obtainable by the method of any one of items 31 to 33.
35. An antibody or antigen-binding fragment as defined in any one of items 1 to 27, which is obtainable by using a production cell line for hypofucosylation or afucosylation.
36. A composition comprising the antibody or antigen-binding fragment according to any one of items 1 to 27, 34 or 35 or the nucleic acid according to item 28, wherein said composition is preferably a pharmaceutical composition.
37. A lipid particle comprising one or more nucleic acids according to item 28.
38. The nucleic acid according to item 28 or the lipid particle according to item 37, wherein the nucleic acid(s) is/are mRNA.
39. A chimeric antigen receptor (CAR) comprising an antibody or antigen-binding fragment as defined in any one of items 1 to 12, 14 or 15, preferably comprising a single-chain variable fragment (scFv) according to any one of items 1 to 12, 14 or 15.
40. A chimeric antigen receptor (CAR) comprising an antigen recognition domain, an extracellular hinge region, a transmembrane domain, and an intracellular signaling domain, wherein said antigen recognition domain is an antibody or antigen-binding fragment according to any one of items 1 to 12, 14 or 15, preferably wherein said antibody or antigen-binding fragment is a single-chain variable fragment (scFv).
41. An immune cell expressing the chimeric antigen receptor according to item 39 or 40.
42. The immune cell according to item 41, which is a T cell or a natural killer cell, preferably a T cell, more preferably an alpha-beta-T cell or a gamma-delta-T cell.
43. The antibody or antigen-binding fragment according to any one of items 1 to 27, 34 or 35, the nucleic acid according to item 28 or 38, the composition according to item 36, the lipid particle according to item 37 or 38, or the immune cell according to item 41 or 42, for use in the treatment of cancer.
44. The antibody or antigen-binding fragment for use according to item 43, the nucleic acid for use according to item 43, the composition for use according to item 43, the lipid particle for use according to item 43, or the immune cell for use according to item 43, wherein said cancer is a solid cancer.
45. The antibody or antigen-binding fragment for use according to item 43, the nucleic acid for use according to item 43, the composition for use according to item 43, the lipid particle for use according to item 43, or the immune cell for use according to item 43, wherein said cancer is a hematological cancer.
46. The antibody or antigen-binding fragment for use according to item 43, the nucleic acid for use according to item 43, the composition for use according to item 43, the lipid particle for use according to item 43, or the immune cell for use according to item 43, wherein said cancer is selected from ovarian cancer, colorectal cancer, colon cancer, gastric cancer, esophageal cancer, breast cancer, lung cancer, bladder cancer, uterine cancer, urothelial cancer, Kaposi's sarcoma, skin cancer, head and/or neck cancer, renal cancer, and lymphoma.
47. The antibody or antigen-binding fragment for use according to any one of items 43 to 46, the nucleic acid for use according to any one of items 43 to 46, the composition for use according to any one of items 43 to 46, the lipid particle for use according to any one of items 43 to 46, or the immune cell for use according to any one of items 43 to 46, wherein said treatment comprises the combined administration of one or more immune checkpoint inhibitors; preferably wherein said one or more immune checkpoint inhibitors are selected from anti-CTLA-4 antibodies, anti-PD-1 antibodies, anti-PD-L1 antibodies, anti-TIGIT antibodies, anti-TIM3 antibodies, anti-LAG3 antibodies, anti-OX40 antibodies, and anti-ICOS antibodies; more preferably wherein said one or more immune checkpoint inhibitors are selected from ipilimumab, tremelimumab, nivolumab, pembrolizumab, pidilizumab, cemiplimab, dostarlimab, spartalizumab, camrelizumab, sintilimab, tislelizumab, toripalimab, zimberelimab, AMP-224, AMP-514, JTX-4014, INCMGA00012, APE02058, atezolizumab, avelumab, durvalumab, KN035, CK-301, BMS-936559, MEDI4736, MPDL3280A, MDX-1105, MEDI6469, bintrafusp alfa, tiragolumab, vibostolimab, domvanalimab, etigilimab, BMS-986207, EOS-448, COM902, ASP8374, SEA-TGT, BGB-A1217, IBI939, M6223, cobolimab, sabatolimab, BMS-986258, Sym023, TQB2618, LY3321367, SHR-1702, relatlimab, ieramilimab, encelimab, tebotelimab, REGN3767, FS118, IMP701, IMP731, ivuxolimab, MEDI0562, MEDI6383, MEDI6469, INCAGN01949, ABBV-368, BAT6026, BGB-A445, YH-002, BMS 986178, INBRX-106, IBI101, MOXR0916, alomfilimab, feladilimab, vopratelimab, BMS-986226, MEDI-570, and XmAb23104.

The invention will now be described by reference to the following examples which are merely illustrative and are not to be construed as a limitation of the scope of the present invention.

### EXAMPLES

### Abbreviations

ADCC: antibody-dependent cellular cytotoxicity
ADCP: antibody-dependent cellular phagocytosis
CD: cluster of differentiation
DIEA: N,N-diisopropylethylamine
DMF: dimethylformamide
EDTA: ethylenediaminetetraacetic acid
ELISA: enzyme-linked immunosorbent assay
ESI-HRMS: electrospray ionization high-resolution mass spectrometry
FBS: fetal bovine serum
FcR: Fc receptor
Fmoc protecting group: fluorenylmethoxycarbonyl protecting group
GM-CSF: granulocyte macrophage colony-stimulating factor
HCTU: O-(1H-6-chlorobenzotriazole-1-yl)-1,1,3,3-tetramethyluronium hexafluorophosphate
HPLC: high-performance liquid chromatography
HTRF: homogeneous time resolved fluorescence
HRP: horseradish peroxidase
IL: interleukin
mAb: monoclonal antibody
MDM: monocyte-derived macrophages
Mono: monocytes
NK: natural killer
NMP: N-methyl-2-pyrrolidone
OD: optical density
PBMC: peripheral blood mononuclear cells
PBS: phosphate-buffered saline
SD: standard deviation
TMB: 3,3′,5,5′-tetramethylbenzidine
Treg: T regulatory cells
Tris: tris(hydroxymethyl)aminomethane

### Example 1: Generation of Unmodified or Modified CCR8 Related Peptides

A peptide corresponding to a 34 amino acid sequence from the N-terminal extracellular domain of human CCR8 as well as modified versions of this peptide having one or more sulfated tyrosine residues were prepared as described in the following.

The peptides were generated by SB-peptides (Saint Egreve, France) or by Pepscan (Lelystad, The Netherlands). The peptides were assembled stepwise using Fmoc-based Solid Phase Peptide Synthesis (SPPS) on a PTI Symphony synthesizer on resin. The Fmoc protecting group was removed using 20% piperidine in DMF and free amine was coupled using tenfold excess of Fmoc amino acids and HCTU/DIEA activation in NMP/DMF (3×15 min). Peptides were de-protected and cleaved from the resin with cleavage cocktail, then precipitated out in cold diethyl ether. The resulting white solids were washed twice with diethyl ether and re-suspended. Purification of the peptides was performed on RP-HPLC C18 column. Peptides have been controlled by ESI-HRMS and HPLC on Agilent systems, then freeze-dried.

Table 1 shows the list of CCR8 related peptide sequences according to tyrosine sulfation or not at the position Tyr 15, Tyr 16 or Tyr 17. The amino acid sequence of the N-terminal extracellular domain of human CCR8 comprises the amino acid sequence MDYTLDLSVTTVTDYYYPDIFSSPCDAELIQTNG (SEQ ID NO: 116) defined as the unmodified peptide (pwt1). The other modified peptides (pm1, pm2, pm3, pm4, pm5, pm6 or pm8) contain at least one, two or three tyrosine sulfation amino acids at the position Tyr 15, Tyr 16 or Tyr 17.

TABLE 1
List of CCR8 related peptide sequences according
tyrosine sulfation (underlined Y = sulfated
tyrosine residue)

| Codification | Sequence |
|---|---|
| pwt1 | MDYTLDLSVTTVTDYYYPDIFSSPCDAELIQTNG |
| | (SEQ ID NO: 116) |
| pm1 | MDYTLDLSVTTVTDYYYPDIFSSPCDAELIQTNG |
| | (SEQ ID NO: 117) |
| pm2 | MDYTLDLSVTTVTDYYYPDIFSSPCDAELIQTNG |
| | (SEQ ID NO: 118) |
| pm3 | MDYTLDLSVTTVTDYYYPDIFSSPCDAELIQTNG |
| | (SEQ ID NO: 2) |
| pm4 | MDYTLDLSVTTVTDYYYPDIFSSPCDAELIQTNG |
| | (SEQ ID NO: 119) |
| pm5 | MDYTLDLSVTTVTDYYYPDIFSSPCDAELIQTNG |
| | (SEQ ID NO: 3) |
| pm6 | MDYTLDLSVTTVTDYYYPDIFSSPCDAELIQTNG |
| | (SEQ ID NO: 4) |
| pm8 | MDYTLDLSVTTVTDYYYPDIFSSPCDAELIQTNG |
| | (SEQ ID NO: 5) |

### Example 2: Preparation of Comparative Benchmark Antibodies

### Ref #1

Nucleotide sequences encoding the VH and VL region from the human CCR8 specific antibody “Ref #1” were retrieved from the patent application WO 2021/260210, specifically from the description of the antibody “TPP-21360” (the nucleotide sequences encoding the VH region and the VL region of TPP-21360 are disclosed in the sequence listing of WO 2021/260210 as sequence ID numbers 607 and 611, respectively, which are incorporated herein by reference; the amino acid sequence of the VH region of this benchmark antibody “Ref #1” is EVQLLESGGGLVQPGGSLRLSCAASGFTFSSYGMHWVRQAPGKGLEWVSAINWNGGSTGYADSVKGRFTISRDN SKNTLYLQMNSLRAEDTAVYYCARGHHSGYDGRFFDYWGQGTLVTVSS (SEQ ID NO: 120), and the amino acid sequence of its VL region is QSVLTQPPSVSGAPGQRVTISCTGSSSNIGAGYNVHWYQQLPGTAPKLLIYTNNRRP SGVPDRFSGSKSGTSASLAISGLRSEDEADYYCAAWDASLSGWVFGGGTKLTVL (SEQ ID NO: 121), which are also disclosed in the sequence listing of WO 2021/260210 as sequence ID numbers 599 and 603, respectively). Nucleotide sequences were gene synthesized as linear DNA fragments with appropriate flanking regions (suitable restriction enzyme recognition sites, linker sequences) (RD Biotech, Besançon, France). The DNA fragments were cloned into suitable mammalian IgG expression vectors encoding heavy and light chains of human IgG1. The humanized mAb Ref #1 was transiently produced in CHO cell line.

### Ref #2

Nucleotide sequences encoding the VH and VL region from the human CCR8 specific antibody “Ref #2” were retrieved from the patent application US 2021/0238292 A1, specifically from the description of the antibody “Anti-CCR8-1” (the amino acid sequence of the VH region of this benchmark antibody is “Ref #2” QVQLVQSGAEVKKPGASVKVSCKASGYTFTSYYMHWVRQAPGQGLEWMGIINPSGGSTSYAQKFQGRVTMTRDT STSTVYMELSSLRSEDTAVYYCARAVRNRFRFDYWGQGTLVTVSS (SEQ ID NO: 155), and the amino acid sequence of its VL region is QSALTQPASVSGSPGQSITISCTGTSSDVGSYNLVSWYQQHPGKAPKLMIYEVSKRPSGVSNRFSGSKSGNTASLT ISGLQAEDEADYYCSSYAGSSTFVVFGGGTKLTVL (SEQ ID NO: 156), which are also disclosed in the sequence listing of US 2021/0238292 A1 as sequence ID numbers 41 and 43, respectively). Nucleotide sequences were gene synthesized as linear DNA fragments with appropriate flanking regions (suitable restriction enzyme recognition sites, linker sequences) (RD Biotech, Besançon, France). The DNA fragments were cloned into suitable mammalian IgG expression vectors encoding heavy and light chains of human IgG1. The humanized mAb Ref #2 was transiently produced in CHO cell line.

### Ref #3

Nucleotide sequences encoding the VH and VL region from the human CCR8 specific antibody “Ref #3” were retrieved from the patent application WO 2022/078277, specifically from the description of the antibody “137-H3L2 (LM-108)” (the amino acid sequence of the VH region of this benchmark antibody “Ref #3” is EVOLVESGGGLVQPGGSLKLSCAASGFTFNTYAMNWVRQASGKGLEWVARIRSKANNYATYYADSVKDRFTISRD DSKNTLYLQMNNLKTEDTAVYYCVRDRSRGEDYAMDYWGQGTLVTVSS (SEQ ID NO: 157), and the amino acid sequence of its VL region is DIVMTQSPLSLPVTPGEPASISCRSSKSLLHSNANTYLYWFLQKPGQSPQLLIYRMSNLASGVPDRFSGSGSGTAFT LKISRVEAEDVGVYYCMQHLEYPFTFGQGTKLEIK (SEQ ID NO: 158), which are also disclosed in the sequence listing of WO 2022/078277 as sequence ID numbers 40 and 42, respectively). Nucleotide sequences were gene synthesized as linear DNA fragments with appropriate flanking regions (suitable restriction enzyme recognition sites, linker sequences) (RD Biotech, Besançon, France). The DNA fragments were cloned into suitable mammalian IgG expression vectors encoding heavy and light chains of human IgG1. The humanized mAb Ref #3 was transiently produced in CHO cell line.

### Example 3: Additional Control Antibodies

Different isotype “negative control” antibodies not targeting CCR8 were used according to the mAb format. For example, hIgG1 (catalog ref. 403502, Clone QA16A12, Biolegend, United Kingdom) was used as humanized negative control. As mouse isotype control mAb, mouse IgG1 (catalog ref. 401408, Clone MG1-45, Biolegend, United Kingdom), mouse IgG2a (catalog ref. 401508, Clone MG2a-55, Biolegend, United Kingdom), mouse IgG2b (catalog ref. 401216, Clone MG2b-57, Biolegend, United Kingdom) or mouse IgG3 (catalog ref. 401302, Clone MG3-35, Biolegend, United Kingdom) was used.

Rituximab (batch no. N7174B06, Evidentic, Berlin, Germany), a chimeric mAb targeting CD20, was used as negative control in the Antibody-Dependent Cellular Phagocytosis assay.

Lacutamab, a mAb targeting KIR3DL2, was used as positive control in the Antibody-Dependent Cellular Cytotoxicity assay and in the Antibody-Dependent Cellular Phagocytosis assay. The amino acid sequences of this antibody were retrieved from the IMGT mAb-DB database (IMGT/mAb-DB ID519). Nucleotide sequences were gene synthesized as linear DNA fragments with appropriate flanking regions (suitable restriction enzyme recognition sites, linker sequences) (RD Biotech, Besançon, France). The DNA fragments were cloned into suitable expression vector. The humanized mAb (lacutamab) was transiently produced in CHO cell line over 2 weeks. The antibody was purified by one step affinity chromatography on protein A and dialyzed in PBS pH 7.4.

The murine anti-human CCR8 “positive control” monoclonal antibody L263G8 (Biolegend, San Diego, California) was also used in the different studies.

### Example 4: Generation of Monoclonal Antibodies Targeting CCR8

The anti-CCR8 antibodies, i.e. murine monoclonal antibodies specific for human CCR8, were produced using standard hybridoma techniques (Zola H, “Monoclonal Antibodies: A Manual of Techniques”, CRC Press, 1987, doi: 10.1201/9781482242683). Briefly, mice received i.p. injections of peptides corresponding to the N-terminal part of the human CCR8 sequence, and more precisely to the first 34 amino acids. The splenocytes were fused with mouse myeloma cell line X63-Ag8.653. Hybridoma supernatants were screened for human CCR8 by ELISA against peptides and by flow cytometry on CHO-K1 cell line expressing in a recombinant manner human CCR8.

### Example 5: Purification of CCR8 Binders from Supernatants of Hybridoma Post Dilution Limit

Protein A chromatography from hybridoma supernatants: The hybridoma supernatants were adjusted to pH 8.3 with the equilibration buffer 0.1 M Tris and 1.5 M ammonium sulfate and then loaded onto the Protein A Sepharose Fast Flow column (GE Healthcare, Saint Cyr au Mont d'or, France). The non-binding proteins are flowed through and removed by several washings with equilibration buffer. The anti-CCR8 mAb is eluted off the Protein A column using the elution buffer 0.1 M sodium citrate at pH 3.5. Column eluent is monitored by measuring absorbance at 280 nm.

The amino acid residues in CDRs of the heavy variable domain sequence and CDRs of the light variable domain sequence of each mAb are determined using a combined definition rule by using IMGT® database. Table 2.1 shows the list of CDR sequences for each individual mAb obtained in this example.

TABLE 2.1
List of CDR sequences of anti-CCR8 mAbs
(analyzed using the IMGT® database)

| | SEQ | Sequence |
|---|---|---|
| 1-1 | ID NO: | IMGT® |

VH

| | | |
|---|---|---|
| CDR1 | 6 | PYAMN |
| CDR2 | 7 | RIRSKSNNYATYYADSVKD |
| CDR3 | 8 | GREAYYRYDGDYYAMDY |

VL

| CDR1 | 9 | RSSKSLLHSNGNTYLY |
|---|---|---|
| CDR2 | 10 | RMSNLAS |
| CDR3 | 11 | MQHLEYPFT |
| | SEQ | Sequence |
| 1-2 | ID NO: | IMGT® |

VH

| | | |
|---|---|---|
| CDR1 | 12 | TYAMN |
| CDR2 | 7 | RIRSKSNNYATYYADSVKD |
| CDR3 | 13 | GREAYYRYDGGYYAMDY |

VL

| CDR1 | 9 | RSSKSLLHSNGNTYLY |
|---|---|---|
| CDR2 | 10 | RMSNLAS |
| CDR3 | 11 | MQHLEYPFT |
| | SEQ | Sequence |
| 1-3 | ID NO: | IMGT® |

VH

| | | |
|---|---|---|
| CDR1 | 14 | EYTIH |
| CDR2 | 15 | GINPNGDTRYDQKFKG |
| CDR3 | 16 | VARFYGISPYAMDY |

VL

| CDR1 | 17 | RCTQSLLHSNGDTYLH |
|---|---|---|
| CDR2 | 18 | KVSNRFS |
| CDR3 | 19 | SQSTHVPYT |
| | SEQ | Sequence |
| 1-4 | ID NO: | IMGT® |

VH

| | | |
|---|---|---|
| CDR1 | 20 | AYAMN |
| CDR2 | 21 | RIRSKSNDYATYYGDSVKD |
| CDR3 | 13 | GREAYYRYDGGYYAMDY |

VL

| CDR1 | 22 | TSSKSLLHSNGNTYLY |
|---|---|---|
| CDR2 | 10 | RMSNLAS |
| CDR3 | 23 | MQHREYPFT |
| | SEQ | Sequence |
| 1-5 | ID NO: | IMGT® |

VH

| | | |
|---|---|---|
| CDR1 | 24 | DYVVT |
| CDR2 | 25 | VIWGGGNTYYNSDLKS |
| CDR3 | 26 | RHRDYALDY |

VL

| CDR1 | 27 | RSSQSLVYSNGNTYLH |
|---|---|---|
| CDR2 | 18 | KVSNRFS |
| CDR3 | 28 | SQSTYVPPT |
| | SEQ | Sequence |
| 1-6 | ID NO: | IMGT® |

VH

| | | |
|---|---|---|
| CDR1 | 29 | DYTLH |
| CDR2 | 30 | GITPKNGDTRYDPRFKD |
| CDR3 | 31 | VARFYGVSPYAMDY |

VL

| CDR1 | 17 | RCTQSLLHSNGDTYLH |
|---|---|---|
| CDR2 | 18 | KVSNRFS |
| CDR3 | 32 | SQTTHVPYT |
| | SEQ | Sequence |
| 1-7 | ID NO: | IMGT® |

VH

| | | |
|---|---|---|
| CDR1 | 33 | NYRMH |
| CDR2 | 34 | VIKVKSDNYGANYAESVKG |
| CDR3 | 35 | PDGFSPFVY |

VL

| CDR1 | 36 | KASQDINSYLS |
|---|---|---|
| CDR2 | 37 | RANRLVD |
| CDR3 | 38 | LQYGEFPPT |
| 1-8 | SEQ | Sequence |
| | ID NO: | IMGT® |

VH

| | | |
|---|---|---|
| CDR1 | 20 | AYAMN |
| CDR2 | 39 | RIRSKSNNYATYYGDSVKD |
| CDR3 | 8 | GREAYYRYDGDYYAMDY |

VL

| CDR1 | 40 | KSSQSLLNSSNQKNYLA |
|---|---|---|
| CDR2 | 41 | FASTRES |
| CDR3 | 42 | QQHYSTPYT |
| | SEQ | Sequence |
| 1-9 | ID NO: | IMGT® |

VH

| | | |
|---|---|---|
| CDR1 | 20 | AYAMN |
| CDR2 | 7 | RIRSKSNNYATYYADSVKD |
| CDR3 | 13 | GREAYYRYDGGYYAMDY |

VL

| CDR1 | 9 | RSSKSLLHSNGNTYLY |
|---|---|---|
| CDR2 | 10 | RMSNLAS |
| CDR3 | 11 | MQHLEYPFT |
| 1-10 | SEQ | Sequence |
| | ID NO: | IMGT® |

VH

| | | |
|---|---|---|
| CDR1 | 14 | EYTIH |
| CDR2 | 43 | GINPNNGNTRYDQKFKG |
| CDR3 | 44 | VARSSGSGPYAMDY |

VL

| CDR1 | 45 | RCTQSLLHTNGDTYLH |
|---|---|---|
| CDR2 | 18 | KVSNRFS |
| CDR3 | 19 | SQSTHVPYT |
| | SEQ | Sequence |
| 1-11 | ID NO: | IMGT® |

VH

| | | |
|---|---|---|
| CDR1 | 46 | GYNMH |
| CDR2 | 47 | AISPGKGDTSYNLKFKG |
| CDR3 | 48 | SGGTPFAY |

VL

| CDR1 | 49 | RSSQSLVHSNGDTYLY |
|---|---|---|
| CDR2 | 18 | KVSNRFS |
| CDR3 | 19 | SQSTHVPYT |
| | SEQ | Sequence |
| 1-12 | ID NO: | IMGT® |

VH

| | | |
|---|---|---|
| CDR1 | 50 | ANAMN |
| CDR2 | 7 | RIRSKSNNYATYYADSVKD |
| CDR3 | 51 | GSDNYIFYAMDY |

VL

| CDR1 | 9 | RSSKSLLHSNGNTYLY |
|---|---|---|
| CDR2 | 10 | RMSNLAS |
| CDR3 | 11 | MQHLEYPFT |
| | SEQ | Sequence |
| 1-13 | ID NO: | IMGT® |

VH

| | | |
|---|---|---|
| CDR1 | 52 | TNAMN |
| CDR2 | 53 | RIRSKSNYYATYYADSVKD |
| CDR3 | 54 | GREMGNYYSMDY |

VL

| CDR1 | 9 | RSSKSLLHSNGNTYLY |
|---|---|---|
| CDR2 | 10 | RMSNLAS |
| CDR3 | 11 | MQHLEYPFT |
| | SEQ | Sequence |
| 1-14 | ID NO: | IMGT® |

VH

| | | |
|---|---|---|
| CDR1 | 33 | NYRMH |
| CDR2 | 34 | VIKVKSDNYGANYAESVKG |
| CDR3 | 55 | PTYPGSSGFAY |

VL

| CDR1 | 56 | RASQDISNYLN |
|---|---|---|
| CDR2 | 57 | YTSRLHS |
| CDR3 | 58 | QQGNKFPPT |
| | SEQ | Sequence |
| 1-15 | ID NO: | IMGT® |

VH

| | | |
|---|---|---|
| CDR1 | 59 | EYTMH |
| CDR2 | 60 | GINPNNGDTNYNQKFMG |
| CDR3 | 61 | RLLRRGAMDY |

VL

| CDR1 | 62 | RASENVEYYGTSLMQ |
|---|---|---|
| CDR2 | 63 | AASNVDS |
| CDR3 | 64 | QQSRKVPWT |
| | SEQ | Sequence |
| 1-16 | ID NO: | IMGT® |

VH

| | | |
|---|---|---|
| CDR1 | 65 | GSYMH |
| CDR2 | 66 | RINPYNGATSYNQNFKD |
| CDR3 | 67 | TLLRLLDY |

VL

| CDR1 | 68 | RSSKSLLHSNGITYLY |
|---|---|---|
| CDR2 | 69 | QMSNLAS |
| CDR3 | 70 | AQNLELPWT |
| | SEQ | Sequence |
| 1-17 | ID NO: | IMGT® |

VH

| | | |
|---|---|---|
| CDR1 | 71 | SYWMH |
| CDR2 | 72 | NIWPGSASTNYDEKFKN |
| CDR3 | 73 | GGKGAMDY |

VL

| CDR1 | 74 | RSSQSLENSYGNTYLN |
|---|---|---|
| CDR2 | 75 | RVSNRFS |
| CDR3 | 76 | LQVTHVPPT |
| | SEQ | Sequence |
| 1-18 | ID NO: | IMGT® |

VH

| | | |
|---|---|---|
| CDR1 | 77 | TYAMH |
| CDR2 | 78 | HINPSSGYSNYNQKFKD |
| CDR3 | 79 | SEVRRGYFDV |

VL

| | | |
|---|---|---|
| CDR1 | 80 | RTSQDIRNYLN |
| CDR2 | 57 | YTSRLHS |
| CDR3 | 81 | QQGNTLPPT |

### Example 6: Generation of Chimerized, Humanized, and Fc-Optimized Anti-CCR8 Monoclonal Antibodies

### Chimerization

The chimeric antibodies were generated by traditional approaches of molecular and cell biology. Each murine variable region was synthetized as cDNA and subcloned into an expression vector containing the human constant region C kappa or human IgG1. The cloning was confirmed by sequencing prior the antibody production. The productions were done in CHO cell line by transient transfection under serum free condition. After two weeks, supernatants were harvested and the antibodies were purified by a one-step protein A chromatography. The quality of the purified antibodies was confirmed by SDS-PAGE and SEC-HPLC analysis and for endotoxin.

### Humanization

The humanization procedure of the variable regions was performed by CDR-grafting with the help of 3D modelling (Yumab GmbH). Briefly, for each murine variable sequence, the most appropriate human acceptor framework was selected based on different criteria: homology with the murine framework, developability including post-translational modifications (PTM) and pairing with the counterpart variable sequence. Then each amino acid diverging between human and murine counterparts was analysed and its impact on a 3D model was evaluated. As a result, different variable sequences were generated for both heavy and light chain, 3 to 4 sequences for each variable region. Like previously, the variable sequences were subcloned in expression vectors and the coding sequences were validated by sequencing.

The production of the humanized and the chimeric antibodies, resulting in the combinatorial of one heavy and one light chain expression vector, was done in CHO cell line by transient transfection under serum free conditions. After one week, supernatant was harvested and the antibodies were purified by a one-step protein A chromatography. The quality of the purified antibodies was confirmed by SDS-PAGE and SEC-HPLC analysis. Finally, the antigen binding capacity of each antibody was evaluated in dose response by flow cytometry on CHOK1 cell line expressing hCCR8 and compared to the response provided by the chimeric antibody. Untransfected CHO was used to evaluate the unspecific binding of the antibodies.

### Fc Optimization

mAb 1-21 has been designed by combining one of the best humanized variable regions and the most effective Fc fragment for functional activity which derives from a human IgG1 allotype G1m17 having the terminal lysine deleted and containing the SDIE mutations. Fc engineering was based on combination of mutations that are known to improve antibody functionality (ADCC/ADCP/CDC) and that have been used in antibodies tested in clinic. The mutations were introduced in the cDNA coding sequence during gene synthesis or by point mutation and the coding sequence was then sequenced by the Sanger method and compared to the theoretical sequence.

### Preparation of Afucosylated mAb

The glycoengineered antibodies were produced as afucosylated antibodies in transient transfection using fucosyltransferase inhibitor 2-deoxy-2-fluoro-L-fucose which is added 4 hours after transfection at 100 μM. The low fucose content has been validated on purified control antibody by LC-MS in a middle-up approach.

The sequences of the antibodies thus obtained—i.e., the humanized mAbs 1-19, 1-20, 1-21, 1-22, 1-23, 1-24 and 1-25, the afucosylated humanized mAb 1-26, the chimeric mAbs 1-27, 1-28, 1-30, 1-31, 1-32, 1-33, 1-34, 1-36, 1-37, 1-38, 1-39, 1-40, 1-42, 1-43, 1-44, 1-45, 1-46, 1-48, 1-49 and 1-50, and the hypofucosylated (low fucosylated) chimeric mAbs 1-29, 1-35, 1-41 and 1-47—are summarized in the following Tables 2.2 to 2.4:

TABLE 2.2
List of CDR sequences of
anti-CCR8 mAbs 1-19 to 1-50

| | SEQ | Sequence |
|---|---|---|
| 1-19 | ID NO: | IMGT® |

VH

| | | |
|---|---|---|
| CDR1 | 122 | GFTFNTYA |
| CDR2 | 123 | IRSKSNNYAT |
| CDR3 | 124 | VRGREAYYRYDGGYYAMDV |

VL

| CDR1 | 125 | KSLLHSNGNTY |
|---|---|---|
| CDR2 | — | RMS |
| CDR3 | 11 | MQHLEYPFT |
| | SEQ | Sequence |
| 1-20 | ID NO: | IMGT® |

VH

| | | |
|---|---|---|
| CDR1 | 126 | GFTFNAYA |
| CDR2 | 127 | IRSKSNDYAT |
| CDR3 | 124 | VRGREAYYRYDGGYYAMDV |

VL

| CDR1 | 125 | KSLLHSNGNTY |
|---|---|---|
| CDR2 | — | RMS |
| CDR3 | 23 | MQHREYPFT |
| | SEQ | Sequence |
| 1-21 | ID NO: | IMGT® |

VH

| | | |
|---|---|---|
| CDR1 | 126 | GFTFNAYA |
| CDR2 | 127 | IRSKSNDYAT |
| CDR3 | 124 | VRGREAYYRYDGGYYAMDV |

VL

| CDR1 | 125 | KSLLHSNGNTY |
|---|---|---|
| CDR2 | — | RMS |
| CDR3 | 23 | MQHREYPFT |
| | SEQ | Sequence |
| 1-22 | ID NO: | IMGT® |

VH

| | | |
|---|---|---|
| CDR1 | 126 | GFTFNAYA |
| CDR2 | 127 | IRSKSNDYAT |
| CDR3 | 124 | VRGREAYYRYDGGYYAMDV |

VL

| CDR1 | 125 | KSLLHSNGNTY |
|---|---|---|
| CDR2 | | RVS |
| CDR3 | 23 | MQHREYPFT |
| | SEQ | Sequence |
| 1-23 | ID NO: | IMGT® |

VH

| | | |
|---|---|---|
| CDR1 | 128 | GYTFTEYT |
| CDR2 | 129 | INPNNGNT |
| CDR3 | 130 | ARVARSSGSGPYAMDY |

VL

| CDR1 | 131 | QSLLHTNGDTY |
|---|---|---|
| CDR2 | — | KVS |
| CDR3 | 19 | SQSTHVPYT |
| | SEQ | Sequence |
| 1-24 | ID NO: | IMGT® |
| VH | | |
| CDR1 | 128 | GYTFTEYT |
| CDR2 | 129 | INPNNGNT |
| CDR3 | 130 | ARVARSSGSGPYAMDY |
| VL | | |
| CDR1 | 131 | QSLLHTNGDTY |
| CDR2 | — | KVS |
| CDR3 | 19 | SQSTHVPYT |
| | SEQ | Sequence |
| 1-25 | ID NO: | IMGT® |

VH

| | | |
|---|---|---|
| CDR1 | 134 | GFTFSNYR |
| CDR2 | 135 | IKVKSDNYGA |
| CDR3 | 136 | SSPTYPGSSGFAY |

VL

| | | |
|---|---|---|
| CDR1 | 137 | QDISNY |
| CDR2 | | YTS |
| CDR3 | 58 | QQGNKFPPT |
| | SEQ | Sequence |
| 1-26 | ID NO: | IMGT® |

VH

| | | |
|---|---|---|
| CDR1 | 126 | GFTFNAYA |
| CDR2 | 127 | IRSKSNDYAT |
| CDR3 | 124 | VRGREAYYRYDGGYYAMDV |

VL

| CDR1 | 125 | KSLLHSNGNTY |
|---|---|---|
| CDR2 | — | RMS |
| CDR3 | 23 | MQHREYPFT |
| | SEQ | Sequence |
| 1-27 | ID NO: | IMGT® |

VH

| | | |
|---|---|---|
| CDR1 | 126 | GFTFNAYA |
| CDR2 | 127 | IRSKSNDYAT |
| CDR3 | 133 | VRGREAYYRYDGGYYAMDY |

VL

| CDR1 | 125 | KSLLHSNGNTY |
|---|---|---|
| CDR2 | — | RMS |
| CDR3 | 23 | MQHREYPFT |
| | SEQ | Sequence |
| 1-28 | ID NO: | IMGT® |

VH

| | | |
|---|---|---|
| CDR1 | 126 | GFTFNAYA |
| CDR2 | 127 | IRSKSNDYAT |
| CDR3 | 133 | VRGREAYYRYDGGYYAMDY |

VL

| CDR1 | 125 | KSLLHSNGNTY |
|---|---|---|
| CDR2 | — | RMS |
| CDR3 | 23 | MQHREYPFT |
| | SEQ | Sequence |
| 1-29 | ID NO: | IMGT® |

VH

| | | |
|---|---|---|
| CDR1 | 126 | GFTFNAYA |
| CDR2 | 127 | IRSKSNDYAT |
| CDR3 | 133 | VRGREAYYRYDGGYYAMDY |

VL

| CDR1 | 125 | KSLLHSNGNTY |
|---|---|---|
| CDR2 | | RMS |
| CDR3 | 23 | MQHREYPFT |
| | SEQ | Sequence |
| 1-30 | ID NO: | IMGT® |
| VH | | |
| CDR1 | 126 | GFTFNAYA |
| CDR2 | 127 | IRSKSNDYAT |
| CDR3 | 133 | VRGREAYYRYDGGYYAMDY |
| VL | | |
| CDR1 | 125 | KSLLHSNGNTY |
| CDR2 | | RMS |
| CDR3 | 23 | MQHREYPFT |
| | SEQ | Sequence |
| 1-31 | ID NO: | IMGT® |
| VH | | |
| CDR1 | 126 | GFTFNAYA |
| CDR2 | 127 | IRSKSNDYAT |
| CDR3 | 133 | VRGREAYYRYDGGYYAMDY |
| VL | | |
| CDR1 | 125 | KSLLHSNGNTY |
| CDR2 | | RMS |
| CDR3 | 23 | MQHREYPFT |
| | SEQ | Sequence |
| 1-32 | ID NO: | IMGT® |

VH

| | | |
|---|---|---|
| CDR1 | 126 | GFTFNAYA |
| CDR2 | 127 | IRSKSNDYAT |
| CDR3 | 133 | VRGREAYYRYDGGYYAMDY |

VL

| CDR1 | 125 | KSLLHSNGNTY |
|---|---|---|
| CDR2 | | RMS |
| CDR3 | 23 | MQHREYPFT |
| | SEQ | Sequence |
| 1-33 | ID NO: | IMGT® |

VH

| | | |
|---|---|---|
| CDR1 | 128 | GYTFTEYT |
| CDR2 | 129 | INPNNGNT |
| CDR3 | 130 | ARVARSSGSGPYAMDY |

VL

| CDR1 | 131 | QSLLHTNGDTY |
|---|---|---|
| CDR2 | — | KVS |
| CDR3 | 19 | SQSTHVPYT |
| | SEQ | Sequence |
| 1-34 | ID NO: | IMGT® |

VH

| | | |
|---|---|---|
| CDR1 | 128 | GYTFTEYT |
| CDR2 | 129 | INPNNGNT |
| CDR3 | 130 | ARVARSSGSGPYAMDY |

VL

| CDR1 | 131 | QSLLHTNGDTY |
|---|---|---|
| CDR2 | — | KVS |
| CDR3 | 19 | SQSTHVPYT |
| | SEQ | Sequence |
| 1-35 | ID NO: | IMGT® |

VH

| | | |
|---|---|---|
| CDR1 | 128 | GYTFTEYT |
| CDR2 | 129 | INPNNGNT |
| CDR3 | 130 | ARVARSSGSGPYAMDY |

VL

| CDR1 | 131 | QSLLHTNGDTY |
|---|---|---|
| CDR2 | — | KVS |
| CDR3 | 19 | SQSTHVPYT |
| | SEQ | Sequence |
| 1-36 | ID NO: | IMGT® |

VH

| | | |
|---|---|---|
| CDR1 | 128 | GYTFTEYT |
| CDR2 | 129 | INPNNGNT |
| CDR3 | 130 | ARVARSSGSGPYAMDY |

VL

| CDR1 | 131 | QSLLHTNGDTY |
|---|---|---|
| CDR2 | — | KVS |
| CDR3 | 19 | SQSTHVPYT |
| | SEQ | Sequence |
| 1-37 | ID NO: | IMGT® |

VH

| | | |
|---|---|---|
| CDR1 | 128 | GYTFTEYT |
| CDR2 | 129 | INPNNGNT |
| CDR3 | 130 | ARVARSSGSGPYAMDY |

VL

| CDR1 | 131 | QSLLHTNGDTY |
|---|---|---|
| CDR2 | — | KVS |
| CDR3 | 19 | SQSTHVPYT |
| | SEQ | Sequence |
| 1-38 | ID NO: | IMGT® |

VH

| | | |
|---|---|---|
| CDR1 | 128 | GYTFTEYT |
| CDR2 | 129 | INPNNGNT |
| CDR3 | 130 | ARVARSSGSGPYAMDY |

VL

| CDR1 | 131 | QSLLHTNGDTY |
|---|---|---|
| CDR2 | — | KVS |
| CDR3 | 19 | SQSTHVPYT |
| | SEQ | Sequence |
| 1-39 | ID NO: | IMGT® |

VH

| | | |
|---|---|---|
| CDR1 | 122 | GFTFNTYA |
| CDR2 | 123 | IRSKSNNYAT |
| CDR3 | 133 | VRGREAYYRYDGGYYAMDY |

VL

| CDR1 | 125 | KSLLHSNGNTY |
|---|---|---|
| CDR2 | — | RMS |
| CDR3 | 11 | MQHLEYPFT |
| | SEQ | Sequence |
| 1-40 | ID NO: | IMGT® |

VH

| | | |
|---|---|---|
| CDR1 | 122 | GFTFNTYA |
| CDR2 | 123 | IRSKSNNYAT |
| CDR3 | 133 | VRGREAYYRYDGGYYAMDY |

VL

| CDR1 | 125 | KSLLHSNGNTY |
|---|---|---|
| CDR2 | — | RMS |
| CDR3 | 11 | MQHLEYPFT |
| | SEQ | Sequence |
| 1-41 | ID NO: | IMGT® |

VH

| | | |
|---|---|---|
| CDR1 | 122 | GFTFNTYA |
| CDR2 | 123 | IRSKSNNYAT |
| CDR3 | 133 | VRGREAYYRYDGGYYAMDY |

VL

| CDR1 | 125 | KSLLHSNGNTY |
|---|---|---|
| CDR2 | — | RMS |
| CDR3 | 11 | MQHLEYPFT |
| | SEQ | Sequence |
| 1-42 | ID NO: | IMGT® |
| VH | | |
| CDR1 | 122 | GFTFNTYA |
| CDR2 | 123 | IRSKSNNYAT |
| CDR3 | 133 | VRGREAYYRYDGGYYAMDY |
| VL | | |
| CDR1 | 125 | KSLLHSNGNTY |
| CDR2 | — | RMS |
| CDR3 | 11 | MQHLEYPFT |
| | SEQ | Sequence |
| 1-43 | ID NO: | IMGT® |

VH

| | | |
|---|---|---|
| CDR1 | 122 | GFTFNTYA |
| CDR2 | 123 | IRSKSNNYAT |
| CDR3 | 133 | VRGREAYYRYDGGYYAMDY |

VL

| CDR1 | 125 | KSLLHSNGNTY |
|---|---|---|
| CDR2 | — | RMS |
| CDR3 | 11 | MQHLEYPFT |
| | SEQ | Sequence |
| 1-44 | ID NO: | IMGT® |

VH

| | | |
|---|---|---|
| CDR1 | 122 | GFTFNTYA |
| CDR2 | 123 | IRSKSNNYAT |
| CDR3 | 133 | VRGREAYYRYDGGYYAMDY |

VL

| CDR1 | 125 | KSLLHSNGNTY |
|---|---|---|
| CDR2 | — | RMS |
| CDR3 | 11 | MQHLEYPFT |
| | SEQ | Sequence |
| 1-45 | ID NO: | IMGT® |

VH

| | | |
|---|---|---|
| CDR1 | 134 | GFTFSNYR |
| CDR2 | 135 | IKVKSDNYGA |
| CDR3 | 136 | SSPTYPGSSGFAY |

VL

| CDR1 | 137 | QDISNY |
|---|---|---|
| CDR2 | — | YTS |
| CDR3 | 58 | QQGNKFPPT |
| | SEQ | Sequence |
| 1-46 | ID NO: | IMGT® |

VH

| | | |
|---|---|---|
| CDR1 | 134 | GFTFSNYR |
| CDR2 | 135 | IKVKSDNYGA |
| CDR3 | 136 | SSPTYPGSSGFAY |

VL

| CDR1 | 137 | QDISNY |
|---|---|---|
| CDR2 | — | YTS |
| CDR3 | 58 | QQGNKFPPT |
| | SEQ | Sequence |
| 1-47 | ID NO: | IMGT® |

VH

| | | |
|---|---|---|
| CDR1 | 134 | GFTFSNYR |
| CDR2 | 135 | IKVKSDNYGA |
| CDR3 | 136 | SSPTYPGSSGFAY |

VL

| CDR1 | 137 | QDISNY |
|---|---|---|
| CDR2 | — | YTS |
| CDR3 | 58 | QQGNKFPPT |
| | SEQ | Sequence |
| 1-48 | ID NO: | IMGT® |

VH

| | | |
|---|---|---|
| CDR1 | 134 | GFTFSNYR |
| CDR2 | 135 | IKVKSDNYGA |
| CDR3 | 136 | SSPTYPGSSGFAY |

VL

| CDR1 | 137 | QDISNY |
|---|---|---|
| CDR2 | — | YTS |
| CDR3 | 58 | QQGNKFPPT |
| | SEQ | Sequence |
| 1-49 | ID NO: | IMGT® |

VH

| | | |
|---|---|---|
| CDR1 | 134 | GFTFSNYR |
| CDR2 | 135 | IKVKSDNYGA |
| CDR3 | 136 | SSPTYPGSSGFAY |

VL

| CDR1 | 137 | QDISNY |
|---|---|---|
| CDR2 | — | YTS |
| CDR3 | 58 | QQGNKFPPT |
| | SEQ | Sequence |
| 1-50 | ID NO: | IMGT® |

VH

| | | |
|---|---|---|
| CDR1 | 134 | GFTFSNYR |
| CDR2 | 135 | IKVKSDNYGA |
| CDR3 | 136 | SSPTYPGSSGFAY |

VL

| | | |
|---|---|---|
| CDR1 | 137 | QDISNY |
| CDR2 | — | YTS |
| CDR3 | 58 | QQGNKFPPT |

TABLE 2.3
List of variable domain sequences of anti-CCR8 mAbs 1-19 to 1-50

| | | SEQ | |
|---|---|---|---|
| | Variable | ID | |
| mAb | domain | NO | Amino acid sequence |
| 1-19 | VH | 138 | EVQLVESGGGLVQPGRSLRLSCTASGFTFNTYAMNWVRQAPGKGL |
| | | | EWVARIRSKSNNYATYYAASVKGRFTISRDDSKSIAYLQMNSLKT |
| | | | EDTAVYYCVRGREAYYRYDGGYYAMDVWGQGTTVTVSS |
| | VL | 139 | DIVMTQAAPSLPVTPGESASISCRSSKSLLHSNGNTYLYWFLQKP |
| | | | GQSPQLLIYRMSNLASGVPDRFSGSGSGTAFTLKISRVEAEDVGV |
| | | | YYCMQHLEYPFTFGSGTKLEIK |
| 1-20 | VH | 140 | EVQLLESGGGLVQPGGSLRLSCAASGFTFNAYAMNWVRQAPGKGL |
| | | | EWVARIRSKSNDYATYYGDSVKGRFTISRDNSKNTLYLQMNSLRA |
| | | | EDTAVYYCVRGREAYYRYDGGYYAMDVWGQGTTVTVSS |
| | VL | 141 | DIVMTQAAPSLSVTPGESASISCTSSKSLLHSNGNTYLYWFLQKP |
| | | | GQSPQLLIYRMSNLASGVPDRFSGSGSGTAFTLKISRVEAEDVGV |
| | | | YYCMQHREYPFTFGSGTKLEIK |
| 1-21 | VH | 140 | EVQLLESGGGLVQPGGSLRLSCAASGFTFNAYAMNWVRQAPGKGL |
| | | | EWVARIRSKSNDYATYYGDSVKGRFTISRDNSKNTLYLQMNSLRA |
| | | | EDTAVYYCVRGREAYYRYDGGYYAMDVWGQGTTVTVSS |
| | VL | 141 | DIVMTQAAPSLSVTPGESASISCTSSKSLLHSNGNTYLYWFLQKP |
| | | | GQSPQLLIYRMSNLASGVPDRFSGSGSGTAFTLKISRVEAEDVGV |
| | | | YYCMQHREYPFTFGSGTKLEIK |
| 1-22 | VH | 140 | EVQLLESGGGLVQPGGSLRLSCAASGFTFNAYAMNWVRQAPGKGL |
| | | | EWVARIRSKSNDYATYYGDSVKGRFTISRDNSKNTLYLQMNSLRA |
| | | | EDTAVYYCVRGREAYYRYDGGYYAMDVWGQGTTVTVSS |
| | VL | 142 | DIVMTQSPLSLPVTPGEPASISCRSSKSLLHSNGNTYLYWFLQKP |
| | | | GQSPQLLIYRVSNLASGVPDRFSGSGSGTDFTLKISRVEAEDVGV |
| | | | YYCMQHREYPFTFGQGTKLEIK |
| 1-23 | VH | 143 | QVQLVQSGPEVVKPGASVKVSCKTSGYTFTEYTIHWVRQAPGQSL |
| | | | EWVGGINPNNGNTRYDQKFQGRVTITRDKSASTAYMELRSLTSED |
| | | | TAVYYCARVARSSGSGPYAMDYWGQGTTVTVSS |
| | VL | 144 | DWVMTQSPLSLPVTLGDQASISCRCSQSLLHTNGDTYLHWYLQRP |
| | | | GQSPRLLIYKVSNRFSGVPDRFSGSGSGTDFTLKISRVEAEDVGV |
| | | | YFCSQSTHVPYTFGGGTKVEIK |
| 1-24 | VH | 145 | QVQLQQSGPEVVKPGASVKVSCKTSGYTFTEYTIHWVRQSHGQSL |
| | | | EWVGGINPNNGNTRYDQKFKGRVTITIDKSSSTAYMELRSLTSED |
| | | | TAVYYCARVARSSGSGPYAMDYWGQGTTVTVSS |
| | VL | 144 | DVVMTQSPLSLPVTLGDQASISCRCSQSLLHTNGDTYLHWYLQRP |
| | | | GQSPRLLIYKVSNRFSGVPDRFSGSGSGTDFTLKISRVEAEDVGV |
| | | | YFCSQSTHVPYTFGGGTKVEIK |
| 1-25 | VH | 146 | QVQLVESGGGVVQPGRSLRLSCAASGFTFSNYRMHWVRQAPGKGL |
| | | | EWIAVIKVKSDNYGANYADSVKGRFTISRDNSKNTLYLQMNSLRA |
| | | | EDTAVYYCSSPTYPGSSGFAYWGQGTLVTVSS |
| | VL | 132 | DIQMTQSPSSLSASVGDRVTITCRASQDISNYLNWYQQKPDGTVK |
| | | | LLIYYTSRLHTGVPSRFSGSGSGTDYTLTISNLQQEDIATYFCQQ |
| | | | GNKFPPTFGGGTKVEIN |
| 1-26 | VH | 140 | EVQLLESGGGLVQPGGSLRLSCAASGFTFNAYAMNWVRQAPGKGL |
| | | | EWVARIRSKSNDYATYYGDSVKGRFTISRDNSKNTLYLQMNSLRA |
| | | | EDTAVYYCVRGREAYYRYDGGYYAMDVWGQGTTVTVSS |
| | VL | 141 | DIVMTQAAPSLSVTPGESASISCTSSKSLLHSNGNTYLYWFLQKP |
| | | | GQSPQLLIYRMSNLASGVPDRFSGSGSGTAFTLKISRVEAEDVGV |
| | | | YYCMQHREYPFTFGSGTKLEIK |
| 1-27 | VH | 147 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSS |
| | VL | 148 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIK |
| 1-28 | VH | 147 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSS |
| | VL | 148 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIK |
| 1-29 | VH | 147 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSS |
| | VL | 148 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIK |
| 1-30 | VH | 147 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSS |
| | VL | 148 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIK |
| 1-31 | VH | 147 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSS |
| | VL | 148 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIK |
| 1-32 | VH | 147 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSS |
| | VL | 148 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIK |
| 1-33 | VH | 149 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSS |
| | VL | 150 | DWVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIK |
| 1-34 | VH | 149 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSS |
| | VL | 150 | DWVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIK |
| 1-35 | VH | 149 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSS |
| | VL | 150 | DWVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIK |
| 1-36 | VH | 149 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSS |
| | VL | 150 | DWVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIK |
| 1-37 | VH | 149 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSS |
| | VL | 150 | DWVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIK |
| 1-38 | VH | 149 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSS |
| | VL | 150 | DWVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIK |
| 1-39 | VH | 151 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSS |
| | VL | 152 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIK |
| 1-40 | VH | 151 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSS |
| | VL | 152 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIK |
| 1-41 | VH | 151 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSS |
| | VL | 152 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIK |
| 1-42 | VH | 151 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSS |
| | VL | 152 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIK |
| 1-43 | VH | 151 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSS |
| | VL | 152 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIK |
| 1-44 | VH | 151 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSS |
| | VL | 152 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIK |
| 1-45 | VH | 153 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSA |
| | VL | 154 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEIN |
| 1-46 | VH | 153 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSA |
| | VL | 154 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEIN |
| 1-47 | VH | 153 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSA |
| | VL | 154 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEIN |
| 1-48 | VH | 153 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSA |
| | VL | 154 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEIN |
| 1-49 | VH | 153 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSA |
| | VL | 154 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEIN |
| 1-50 | VH | 153 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSA |
| | VL | 154 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEIN |

TABLE 2.4
List of complete heavy-chain (HC) and light-chain
(LC) sequences of anti-CCR8 mAbs 1-19 to 1-50

| mAb | Chain | SEQ ID NO | Amino acid sequence |
|---|---|---|---|
| 1-19 | HC | 159 | EVQLVESGGGLVQPGRSLRLSCTASGFTFNTYAMNWVRQAPGKGL |
| | | | EWVARIRSKSNNYATYYAASVKGRFTISRDDSKSIAYLQMNSLKT |
| | | | EDTAVYYCVRGREAYYRYDGGYYAMDVWGQGTTVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPSVFLFPPKPKDTLMISRTPEV |
| | | | TCVVVDVSHEDPEVKFNWYVDGVEVHNAKTKPREEQYNSTYRWSV |
| | | | LTVLHQDWLNGKEYKCKVSNKALPAPIEKTISKAKGQPREPQVYT |
| | | | LPPSRDELTKNQVSLTCLVKGFYPSDIAVEWESNGQPENNYKTTP |
| | | | PVLDSDGSFFLYSKLTVDKSRWQQGNVFSCSVMHEALHNHYTQKS |
| | | | LSLSPG |
| | LC | 160 | DIVMTQAAPSLPVTPGESASISCRSSKSLLHSNGNTYLYWFLQKP |
| | | | GQSPQLLIYRMSNLASGVPDRFSGSGSGTAFTLKISRVEAEDVGV |
| | | | YYCMQHLEYPFTFGSGTKLEIKRTVAAPSVFIFPPSDEQLKSGTA |
| | | | SVVCLLNNFYPREAKVQWKVDNALQSGNSQESVTEQDSKDSTYSL |
| | | | SSTLTLSKADYEKHKVYACEVTHQGLSSPVTKSFNRGEC |
| 1-20 | HC | 161 | EVQLLESGGGLVQPGGSLRLSCAASGFTFNAYAMNWVRQAPGKGL |
| | | | EWVARIRSKSNDYATYYGDSVKGRFTISRDNSKNTLYLQMNSLRA |
| | | | EDTAVYYCVRGREAYYRYDGGYYAMDVWGQGTTVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPSVFLFPPKPKDTLMISRTPEV |
| | | | TCVVVDVSHEDPEVKFNWYVDGVEVHNAKTKPREEQYNSTYRVVS |
| | | | VLTVLHQDWLNGKEYKCKVSNKALPAPIEKTISKAKGQPREPQVY |
| | | | TLPPSRDELTKNQVSLTCLVKGFYPSDIAVEWESNGQPENNYKTT |
| | | | PPVLDSDGSFFLYSKLTVDKSRWQQGNVFSCSVMHEALHNHYTQK |
| | | | SLSLSPG |
| | LC | 162 | DIVMTQAAPSLSVTPGESASISCTSSKSLLHSNGNTYLYWFLQKP |
| | | | GQSPQLLIYRMSNLASGVPDRFSGSGSGTAFTLKISRVEAEDVGV |
| | | | YYCMQHREYPFTFGSGTKLEIKRTVAAPSVFIFPPSDEQLKSGTA |
| | | | SVVCLLNNFYPREAKVQWKVDNALQSGNSQESVTEQDSKDSTYSL |
| | | | SSTLTLSKADYEKHKVYACEVTHQGLSSPVTKSFNRGEC |
| 1-21 | HC | 163 | EVQLLESGGGLVQPGGSLRLSCAASGFTFNAYAMNWVRQAPGKGL |
| | | | EWVARIRSKSNDYATYYGDSVKGRFTISRDNSKNTLYLQMNSLRA |
| | | | EDTAVYYCVRGREAYYRYDGGYYAMDVWGQGTTVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPDVFLFPPKPKDTLMISRTPEV |
| | | | TCVVVDVSHEDPEVKFNWYVDGVEVHNAKTKPREEQYNSTYRVVS |
| | | | VLTVLHQDWLNGKEYKCKVSNKALPAPEEKTISKAKGQPREPQVY |
| | | | TLPPSRDELTKNQVSLTCLVKGFYPSDIAVEWESNGQPENNYKTT |
| | | | PPVLDSDGSFFLYSKLTVDKSRWQQGNVFSCSVMHEALHNHYTQK |
| | | | SLSLSPG |
| | LC | 162 | DIVMTQAAPSLSVTPGESASISCTSSKSLLHSNGNTYLYWFLQKP |
| | | | GQSPQLLIYRMSNLASGVPDRFSGSGSGTAFTLKISRVEAEDVGV |
| | | | YYCMQHREYPFTFGSGTKLEIKRTVAAPSVFIFPPSDEQLKSGTA |
| | | | SVVCLLNNFYPREAKVQWKVDNALQSGNSQESVTEQDSKDSTYSL |
| | | | SSTLTLSKADYEKHKVYACEVTHQGLSSPVTKSFNRGEC |
| 1-22 | HC | 161 | EVQLLESGGGLVQPGGSLRLSCAASGFTFNAYAMNWVRQAPGKGL |
| | | | EWVARIRSKSNDYATYYGDSVKGRFTISRDNSKNTLYLQMNSLRA |
| | | | EDTAVYYCVRGREAYYRYDGGYYAMDVWGQGTTVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPSVFLFPPKPKDTLMISRTPEV |
| | | | TCVVVDVSHEDPEVKFNWYVDGVEVHNAKTKPREEQYNSTYRVVS |
| | | | VLTVLHQDWLNGKEYKCKVSNKALPAPIEKTISKAKGQPREPQVY |
| | | | TLPPSRDELTKNQVSLTCLVKGFYPSDIAVEWESNGQPENNYKTT |
| | | | PPVLDSDGSFFLYSKLTVDKSRWQQGNVFSCSVMHEALHNHYTQK |
| | | | SLSLSPG |
| | LC | 164 | DIVMTQSPLSLPVTPGEPASISCRSSKSLLHSNGNTYLYWFLQKP |
| | | | GQSPQLLIYRVSNLASGVPDRFSGSGSGTDFTLKISRVEAEDVGV |
| | | | YYCMQHREYPFTFGQGTKLEIKRTVAAPSVFIFPPSDEQLKSGTA |
| | | | SVVCLLNNFYPREAKVQWKVDNALQSGNSQESVTEQDSKDSTYSL |
| | | | SSTLTLSKADYEKHKVYACEVTHQGLSSPVTKSFNRGEC |
| 1-23 | HC | 165 | QVQLVQSGPEVVKPGASVKVSCKTSGYTFTEYTIHWVRQAPGQSL |
| | | | EWVGGINPNNGNTRYDQKFQGRVTITRDKSASTAYMELRSLTSED |
| | | | TAVYYCARVARSSGSGPYAMDYWGQGTTVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKKVEPKS |
| | | | CDKTHTCPPCPAPELLGGPSVFLFPPKPKDTLMISRTPEVTCVVV |
| | | | DVSHEDPEVKFNWYVDGVEVHNAKTKPREEQYNSTYRVVSVLTVL |
| | | | HQDWLNGKEYKCKVSNKALPAPIEKTISKAKGQPREPQVYTLPPS |
| | | | RDELTKNQVSLTCLVKGFYPSDIAVEWESNGQPENNYKTTPPVLD |
| | | | SDGSFFLYSKLTVDKSRWQQGNVFSCSVMHEALHNHYTQKSLSLS |
| | | | PG |
| | LC | 166 | DWVMTQSPLSLPVTLGDQASISCRCSQSLLHTNGDTYLHWYLQRP |
| | | | GQSPRLLIYKVSNRFSGVPDRFSGSGSGTDFTLKISRVEAEDVGV |
| | | | YFCSQSTHVPYTFGGGTKVEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-24 | HC | 167 | QVQLQQSGPEVVKPGASVKVSCKTSGYTFTEYTIHWVRQSHGQSL |
| | | | EWVGGINPNNGNTRYDQKFKGRVTITIDKSSSTAYMELRSLTSED |
| | | | TAVYYCARVARSSGSGPYAMDYWGQGTTVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKKVEPKS |
| | | | CDKTHTCPPCPAPELLGGPSVFLFPPKPKDTLMISRTPEVTCVVV |
| | | | DVSHEDPEVKFNWYVDGVEVHNAKTKPREEQYNSTYRVVSVLTVL |
| | | | HQDWLNGKEYKCKVSNKALPAPIEKTISKAKGQPREPQVYTLPPS |
| | | | RDELTKNQVSLTCLVKGFYPSDIAVEWESNGQPENNYKTTPPVLD |
| | | | SDGSFFLYSKLTVDKSRWQQGNVFSCSVMHEALHNHYTQKSLSLS |
| | | | PG |
| | LC | 166 | DVVMTQSPLSLPVTLGDQASISCRCSQSLLHTNGDTYLHWYLQRP |
| | | | GQSPRLLIYKVSNRFSGVPDRFSGSGSGTDFTLKISRVEAEDVGV |
| | | | YFCSQSTHVPYTFGGGTKVEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-25 | HC | 168 | QVQLVESGGGVVQPGRSLRLSCAASGFTFSNYRMHWVRQAPGKGL |
| | | | EWIAVIKVKSDNYGANYADSVKGRFTISRDNSKNTLYLQMNSLRA |
| | | | EDTAVYYCSSPTYPGSSGFAYWGQGTLVTVSSASTKGPSVFPLAP |
| | | | SSKSTSGGTAALGCLVKDYFPEPVTVSWNSGALTSGVHTFPAVLQ |
| | | | SSGLYSLSSWTVPSSSLGTQTYICNVNHKPSNTKVDKKVEPKSCD |
| | | | KTHTCPPCPAPELLGGPSVFLFPPKPKDTLMISRTPEVTCVWVDV |
| | | | SHEDPEVKFNWYVDGVEVHNAKTKPREEQYNSTYRVVSVLTVLHQ |
| | | | DWLNGKEYKCKVSNKALPAPIEKTISKAKGQPREPQVYTLPPSRD |
| | | | ELTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPG |
| | LC | 169 | DIQMTQSPSSLSASVGDRVTITCRASQDISNYLNWYQQKPDGTVK |
| | | | LLIYYTSRLHTGVPSRFSGSGSGTDYTLTISNLQQEDIATYFCQQ |
| | | | GNKFPPTFGGGTKVEINRTVAAPSVFIFPPSDEQLKSGTASVVCL |
| | | | LNNFYPREAKVQWKVDNALQSGNSQESVTEQDSKDSTYSLSSTLT |
| | | | LSKADYEKHKVYACEVTHQGLSSPVTKSFNRGEC |
| 1-26 | HC | 163 | EVQLLESGGGLVQPGGSLRLSCAASGFTFNAYAMNWVRQAPGKGL |
| | | | EWVARIRSKSNDYATYYGDSVKGRFTISRDNSKNTLYLQMNSLRA |
| | | | EDTAVYYCVRGREAYYRYDGGYYAMDVWGQGTTVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPDVFLFPPKPKDTLMISRTPEV |
| | | | TCVVVDVSHEDPEVKFNWYVDGVEVHNAKTKPREEQYNSTYRVVS |
| | | | VLTVLHQDWLNGKEYKCKVSNKALPAPEEKTISKAKGQPREPQVY |
| | | | TLPPSRDELTKNQVSLTCLVKGFYPSDIAVEWESNGQPENNYKTT |
| | | | PPVLDSDGSFFLYSKLTVDKSRWQQGNVFSCSVMHEALHNHYTQK |
| | | | SLSLSPG |
| | LC | 162 | DIVMTQAAPSLSVTPGESASISCTSSKSLLHSNGNTYLYWFLQKP |
| | | | GQSPQLLIYRMSNLASGVPDRFSGSGSGTAFTLKISRVEAEDVGV |
| | | | YYCMQHREYPFTFGSGTKLEIKRTVAAPSVFIFPPSDEQLKSGTA |
| | | | SVVCLLNNFYPREAKVQWKVDNALQSGNSQESVTEQDSKDSTYSL |
| | | | SSTLTLSKADYEKHKVYACEVTHQGLSSPVTKSFNRGEC |
| 1-27 | HC | 170 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPSVFLFPPKPKDTLMISRTPEV |
| | | | TCVVVDVSHEDPEVKFNWYVDGVEVHNAKTKPREEQYNSTYRVVS |
| | | | VLTVLHQDWLNGKEYKCKVSNKALPAPIEKTISKAKGQPREPQVY |
| | | | TLPPSRDELTKNQVSLTCLVKGFYPSDIAVEWESNGQPENNYKTT |
| | | | PPVLDSDGSFFLYSKLTVDKSRWQQGNVFSCSVMHEALHNHYTQK |
| | | | SLSLSPG |
| | LC | 171 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIKRTVAAPSVFIFPPSDEQLKSGTA |
| | | | SVVCLLNNFYPREAKVQWKVDNALQSGNSQESVTEQDSKDSTYSL |
| | | | SSTLTLSKADYEKHKVYACEVTHQGLSSPVTKSFNRGEC |
| 1-28 | HC | 172 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPDVFLFPPKPKDTLMISRTPEV |
| | | | TCVVVDVSHEDPEVKFNWYVDGVEVHNAKTKPREEQYNSTYRVVS |
| | | | VLTVLHQDWLNGKEYKCKVSNKALPAPEEKTISKAKGQPREPQVY |
| | | | TLPPSRDELTKNQVSLTCLVKGFYPSDIAVEWESNGQPENNYKTT |
| | | | PPVLDSDGSFFLYSKLTVDKSRWQQGNVFSCSVMHEALHNHYTQK |
| | | | SLSLSPG |
| | LC | 171 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIKRTVAAPSVFIFPPSDEQLKSGTA |
| | | | SVVCLLNNFYPREAKVQWKVDNALQSGNSQESVTEQDSKDSTYSL |
| | | | SSTLTLSKADYEKHKVYACEVTHQGLSSPVTKSFNRGEC |
| 1-29 | HC | 170 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPSVFLFPPKPKDTLMISRTPEV |
| | | | TCVVVDVSHEDPEVKFNWYVDGVEVHNAKTKPREEQYNSTYRVVS |
| | | | VLTVLHQDWLNGKEYKCKVSNKALPAPIEKTISKAKGQPREPQVY |
| | | | TLPPSRDELTKNQVSLTCLVKGFYPSDIAVEWESNGQPENNYKTT |
| | | | PPVLDSDGSFFLYSKLTVDKSRWQQGNVFSCSVMHEALHNHYTQK |
| | | | SLSLSPG |
| | LC | 171 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIKRTVAAPSVFIFPPSDEQLKSGTA |
| | | | SVVCLLNNFYPREAKVQWKVDNALQSGNSQESVTEQDSKDSTYSL |
| | | | SSTLTLSKADYEKHKVYACEVTHQGLSSPVTKSFNRGEC |
| 1-30 | HC | 173 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPDVFLFPPKPKDTLMISRTPEV |
| | | | TCVVVDVSHEDPEVKFNWYVDGVEVHNAKTKPREEQYNSTYRVVS |
| | | | VLTVLHQDWLNGKEYKCKVSNKALPLPEEKTISKAKGQPREPQVY |
| | | | TLPPSRDELTKNQVSLTCLVKGFYPSDIAVEWESNGQPENNYKTT |
| | | | PPVLDSDGSFFLYSKLTVDKSRWQQGNVFSCSVMHEALHNHYTQK |
| | | | SLSLSPG |
| | LC | 171 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIKRTVAAPSVFIFPPSDEQLKSGTA |
| | | | SVVCLLNNFYPREAKVQWKVDNALQSGNSQESVTEQDSKDSTYSL |
| | | | SSTLTLSKADYEKHKVYACEVTHQGLSSPVTKSFNRGEC |
| 1-31 | HC | 174 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELVGGPSVFLLPPKPKDTLMISRTPEV |
| | | | TCVVVDVSHEDPEVKFNWYVDGVEVHNAKTKPPEEQYNSTLRVVS |
| | | | VLTVLHQDWLNGKEYKCKVSNKALPAPIEKTISKAKGQPREPQVY |
| | | | TLPPSRDELTKNQVSLTCLVKGFYPSDIAVEWESNGQPENNYKTT |
| | | | PLVLDSDGSFFLYSKLTVDKSRWQQGNVFSCSVMHEALHNHYTQK |
| | | | SLSLSPG |
| | LC | 171 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIKRTVAAPSVFIFPPSDEQLKSGTA |
| | | | SVVCLLNNFYPREAKVQWKVDNALQSGNSQESVTEQDSKDSTYSL |
| | | | SSTLTLSKADYEKHKVYACEVTHQGLSSPVTKSFNRGEC |
| 1-32 | HC | 175 | EVQLVESGGGLVQPTGSLRLSCAASGFTFNAYAMNWVRQAPGKGL |
| | | | EWVARIRSKSNDYATYYGDSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELVGGPSVFLLPPKPKDTLMISRTPEV |
| | | | TCVVVDVSHEDPEVKFNWYVDGVEVHNAKTKPPEEQYNSTLRVVS |
| | | | VLTVLHQDWLNGKEYKCKVSNWALPAPISKTISKAKGQPREPQVY |
| | | | TLPPSRDELTKNQVSLTCLVKGFYPSDIAVEWESNGQPENNYKTT |
| | | | PLVLDSDGSFFLYSKLTVDKSRWQQGNVFSCSVMHEALHNHYTQK |
| | | | SLSLSPG |
| | LC | 171 | DIVMTQAAPSVSVTPGESVSISCTSSKSLLHSNGNTYLYWFLQRP |
| | | | GQSPQLLIYRMSNLASGVPDRFSGSGSGTAFTLRISRVEAEDVGV |
| | | | YYCMQHREYPFTFGSGTKLEIKRTVAAPSVFIFPPSDEQLKSGTA |
| | | | SVVCLLNNFYPREAKVQWKVDNALQSGNSQESVTEQDSKDSTYSL |
| | | | SSTLTLSKADYEKHKVYACEVTHQGLSSPVTKSFNRGEC |
| 1-33 | HC | 176 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSSASTKGPSVFPLA |
| | | | PSSKSTSGGTAALGCLVKDYFPEPVTVSWNSGALTSGVHTFPAVL |
| | | | QSSGLYSLSSWTVPSSSLGTQTYICNVNHKPSNTKVDKKVEPKSC |
| | | | DKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLH |
| | | | QDWLNGKEYKCKVSNKALPAPIEKTISKAKGQPREPQVYTLPPSR |
| | | | DELTKNQVSLTCLVKGFYPSDIAVEWESNGQPENNYKTTPPVLDS |
| | | | DGSFFLYSKLTVDKSRWQQGNVFSCSVMHEALHNHYTQKSLSLSP |
| | | | G |
| | LC | 177 | DWVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-34 | HC | 178 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSSASTKGPSVFPLA |
| | | | PSSKSTSGGTAALGCLVKDYFPEPVTVSWNSGALTSGVHTFPAVL |
| | | | QSSGLYSLSSWTVPSSSLGTQTYICNVNHKPSNTKVDKKVEPKSC |
| | | | DKTHTCPPCPAPELLGGPDVFLFPPKPKDTLMISRTPEVTCWVDV |
| | | | SHEDPEVKFNWYVDGVEVHNAKTKPREEQYNSTYRVVSVLTVLHQ |
| | | | DWLNGKEYKCKVSNKALPAPEEKTISKAKGQPREPQVYTLPPSRD |
| | | | ELTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPG |
| | LC | 177 | DVVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-35 | HC | 176 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSSASTKGPSVFPLA |
| | | | PSSKSTSGGTAALGCLVKDYFPEPVTVSWNSGALTSGVHTFPAVL |
| | | | QSSGLYSLSSWTVPSSSLGTQTYICNVNHKPSNTKVDKKVEPKSC |
| | | | DKTHTCPPCPAPELLGGPSVFLFPPKPKDTLMISRTPEVTCVWVD |
| | | | VSHEDPEVKFNWYVDGVEVHNAKTKPREEQYNSTYRVVSVLTVLH |
| | | | QDWLNGKEYKCKVSNKALPAPIEKTISKAKGQPREPQVYTLPPSR |
| | | | DELTKNQVSLTCLVKGFYPSDIAVEWESNGQPENNYKTTPPVLDS |
| | | | DGSFFLYSKLTVDKSRWQQGNVFSCSVMHEALHNHYTQKSLSLSP |
| | | | G |
| | LC | 177 | DWVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIKRTVAAPSVFIFPPSDEQLKSGTA |
| | | | SWVCLLNNFYPREAKVQWKVDNALQSGNSQESVTEQDSKDSTYSL |
| | | | SSTLTLSKADYEKHKVYACEVTHQGLSSPVTKSFNRGEC |
| 1-36 | HC | 179 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSSASTKGPSVFPLA |
| | | | PSSKSTSGGTAALGCLVKDYFPEPVTVSWNSGALTSGVHTFPAVL |
| | | | QSSGLYSLSSWVTVPSSSLGTQTYICNVNHKPSNTKVDKKVEPKS |
| | | | CDKTHTCPPCPAPELLGGPDVFLFPPKPKDTLMISRTPEVTCWVD |
| | | | VSHEDPEVKFNWYVDGVEVHNAKTKPREEQYNSTYRVVSVLTVLH |
| | | | QDWLNGKEYKCKVSNKALPLPEEKTISKAKGQPREPQVYTLPPSR |
| | | | DELTKNQVSLTCLVKGFYPSDIAVEWESNGQPENNYKTTPPVLDS |
| | | | DGSFFLYSKLTVDKSRWQQGNVFSCSVMHEALHNHYTQKSLSLSP |
| | | | G |
| | LC | 177 | DVVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-37 | HC | 180 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSSASTKGPSVFPLA |
| | | | PSSKSTSGGTAALGCLVKDYFPEPVTVSWNSGALTSGVHTFPAVL |
| | | | QSSGLYSLSSWVTVPSSSLGTQTYICNVNHKPSNTKVDKKVEPKS |
| | | | CDKTHTCPPCPAPELVGGPSVFLLPPKPKDTLMISRTPEVTCVVV |
| | | | DVSHEDPEVKFNWYVDGVEVHNAKTKPPEEQYNSTLRVVSVLTVL |
| | | | HQDWLNGKEYKCKVSNKALPAPIEKTISKAKGQPREPQVYTLPPS |
| | | | RDELTKNQVSLTCLVKGFYPSDIAVEWESNGQPENNYKTTPLVLD |
| | | | SDGSFFLYSKLTVDKSRWQQGNVFSCSVMHEALHNHYTQKSLSLS |
| | | | PG |
| | LC | 177 | DVVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-38 | HC | 181 | EVQLQQSGPELVKPGASVKISCKTSGYTFTEYTIHWVKQSHGESL |
| | | | EWVGGINPNNGNTRYDQKFKGKATLTIDKSSSPAYMELRSLTSED |
| | | | SAVYYCARVARSSGSGPYAMDYWGQGTSVTVSSASTKGPSVFPLA |
| | | | PSSKSTSGGTAALGCLVKDYFPEPVTVSWNSGALTSGVHTFPAVL |
| | | | QSSGLYSLSSWTVPSSSLGTQTYICNVNHKPSNTKVDKKVEPKSC |
| | | | DKTHTCPPCPAPELVGGPSVFLLPPKPKDTLMISRTPEVTCVWVD |
| | | | VSHEDPEVKFNWYVDGVEVHNAKTKPPEEQYNSTLRVVSVLTVLH |
| | | | QDWLNGKEYKCKVSNWALPAPISKTISKAKGQPREPQVYTLPPSR |
| | | | DELTKNQVSLTCLVKGFYPSDIAVEWESNGQPENNYKTTPLVLDS |
| | | | DGSFFLYSKLTVDKSRWQQGNVFSCSVMHEALHNHYTQKSLSLSP |
| | | | G |
| | LC | 177 | DVVMTQTPLSLPVSLGDQASISCRCTQSLLHTNGDTYLHWYLQKP |
| | | | GQSPKLLIYKVSNRFSGVPDRFSGSGSGTDFTLKISRVEAEDLGV |
| | | | YFCSQSTHVPYTFGGGTKLEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-39 | HC | 182 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPSVFLFPPKPKDTLMISRTPEV |
| | | | TCVVVDVSHEDPEVKFNWYVDGVEVHNAKTKPREEQYNSTYRVVS |
| | | | VLTVLHQDWLNGKEYKCKVSNKALPAPIEKTISKAKGQPREPQVY |
| | | | TLPPSRDELTKNQVSLTCLVKGFYPSDIAVEWESNGQPENNYKTT |
| | | | PPVLDSDGSFFLYSKLTVDKSRWQQGNVFSCSVMHEALHNHYTQK |
| | | | SLSLSPG |
| | LC | 183 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIKRTVAAPSVFIFPPSDEQLKSGTA |
| | | | SWVCLLNNFYPREAKVQWKVDNALQSGNSQESVTEQDSKDSTYSL |
| | | | SSTLTLSKADYEKHKVYACEVTHQGLSSPVTKSFNRGEC |
| 1-40 | HC | 184 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPDVFLFPPKPKDTLMISRTPEV |
| | | | TCVVVDVSHEDPEVKFNWYVDGVEVHNAKTKPREEQYNSTYRWSV |
| | | | LTVLHQDWLNGKEYKCKVSNKALPAPEEKTISKAKGQPREPQVYT |
| | | | LPPSRDELTKNQVSLTCLVKGFYPSDIAVEWESNGQPENNYKTTP |
| | | | PVLDSDGSFFLYSKLTVDKSRWQQGNVFSCSVMHEALHNHYTQKS |
| | | | LSLSPG |
| | LC | 183 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-41 | HC | 182 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELLGGPSVFLFPPKPKDTLMISRTPEV |
| | | | TCVVVDVSHEDPEVKFNWYVDGVEVHNAKTKPREEQYNSTYRWSV |
| | | | LTVLHQDWLNGKEYKCKVSNKALPAPIEKTISKAKGQPREPQVYT |
| | | | LPPSRDELTKNQVSLTCLVKGFYPSDIAVEWESNGQPENNYKTTP |
| | | | PVLDSDGSFFLYSKLTVDKSRWQQGNVFSCSVMHEALHNHYTQKS |
| | | | LSLSPG |
| | LC | 183 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-42 | HC | 185 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVTVPSSSLGTQTYICNVNHKPSNTKVDKKV |
| | | | EPKSCDKTHTCPPCPAPELLGGPDVFLFPPKPKDTLMISRTPEVT |
| | | | CVVVDVSHEDPEVKFNWYVDGVEVHNAKTKPREEQYNSTYRWSVL |
| | | | TVLHQDWLNGKEYKCKVSNKALPLPEEKTISKAKGQPREPQVYTL |
| | | | PPSRDELTKNQVSLTCLVKGFYPSDIAVEWESNGQPENNYKTTPP |
| | | | VLDSDGSFFLYSKLTVDKSRWQQGNVFSCSVMHEALHNHYTQKSL |
| | | | SLSPG |
| | LC | 183 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-43 | HC | 186 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELVGGPSVFLLPPKPKDTLMISRTPEV |
| | | | TCVVVDVSHEDPEVKFNWYVDGVEVHNAKTKPPEEQYNSTLRVVS |
| | | | VLTVLHQDWLNGKEYKCKVSNKALPAPIEKTISKAKGQPREPQVY |
| | | | TLPPSRDELTKNQVSLTCLVKGFYPSDIAVEWESNGQPENNYKTT |
| | | | PLVLDSDGSFFLYSKLTVDKSRWQQGNVFSCSVMHEALHNHYTQK |
| | | | SLSLSPG |
| | LC | 183 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-44 | HC | 187 | EVQLVESGGGLVQPKGSLKLSCAASGFTFNTYAMNWVRQAPGKGL |
| | | | EWVARIRSKSNNYATYYADSVKDRFTISRDDSQSMLYLQMNNLKT |
| | | | EDTAMYYCVRGREAYYRYDGGYYAMDYWGQGTSVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKK |
| | | | VEPKSCDKTHTCPPCPAPELVGGPSVFLLPPKPKDTLMISRTPEV |
| | | | TCVVVDVSHEDPEVKFNWYVDGVEVHNAKTKPPEEQYNSTLRVVS |
| | | | VLTVLHQDWLNGKEYKCKVSNWALPAPISKTISKAKGQPREPQVY |
| | | | TLPPSRDELTKNQVSLTCLVKGFYPSDIAVEWESNGQPENNYKTT |
| | | | PLVLDSDGSFFLYSKLTVDKSRWQQGNVFSCSVMHEALHNHYTQK |
| | | | SLSLSPG |
| | LC | 183 | DIVMTQAAPSVPVTPGESVSISCRSSKSLLHSNGNTYLYWFLQRP |
| | | | GQSPQLLIYRMSNLASGVPDRFSGSGSGTAFTLRISRVEAEDVGV |
| | | | YYCMQHLEYPFTFGSGTKLEIKRTVAAPSVFIFPPSDEQLKSGTA |
| | | | SWCLLNNFYPREAKVQWKVDNALQSGNSQESVTEQDSKDSTYSLS |
| | | | STLTLSKADYEKHKVYACEVTHQGLSSPVTKSFNRGEC |
| 1-45 | HC | 188 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSAASTKGPSVFPLAP |
| | | | SSKSTSGGTAALGCLVKDYFPEPVTVSWNSGALTSGVHTFPAVLQ |
| | | | SSGLYSLSSWTVPSSSLGTQTYICNVNHKPSNTKVDKKVEPKSCD |
| | | | KTHTCPPCPAPELLGGPSVFLFPPKPKDTLMISRTPEVTCVWVDV |
| | | | SHEDPEVKFNWYVDGVEVHNAKTKPREEQYNSTYRVVSVLTVLHQ |
| | | | DWLNGKEYKCKVSNKALPAPIEKTISKAKGQPREPQVYTLPPSRD |
| | | | ELTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPG |
| | LC | 189 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEINRTVAAPSVFIFPPSDEQLKSGTASWCLL |
| | | | NNFYPREAKVQWKVDNALQSGNSQESVTEQDSKDSTYSLSSTLTL |
| | | | SKADYEKHKVYACEVTHQGLSSPVTKSFNRGEC |
| 1-46 | HC | 190 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSAASTKGPSVFPLAP |
| | | | SSKSTSGGTAALGCLVKDYFPEPVTVSWNSGALTSGVHTFPAVLQ |
| | | | SSGLYSLSSWTVPSSSLGTQTYICNVNHKPSNTKVDKKVEPKSCD |
| | | | KTHTCPPCPAPELLGGPDVFLFPPKPKDTLMISRTPEVTCWVDVS |
| | | | HEDPEVKFNWYVDGVEVHNAKTKPREEQYNSTYRVVSVLTVLHQD |
| | | | WLNGKEYKCKVSNKALPAPEEKTISKAKGQPREPQVYTLPPSRDE |
| | | | LTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPG |
| | LC | 189 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEINRTVAAPSVFIFPPSDEQLKSGTASWCLL |
| | | | NNFYPREAKVQWKVDNALQSGNSQESVTEQDSKDSTYSLSSTLTL |
| | | | SKADYEKHKVYACEVTHQGLSSPVTKSFNRGEC |
| 1-47 | HC | 188 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSAASTKGPSVFPLAP |
| | | | SSKSTSGGTAALGCLVKDYFPEPVTVSWNSGALTSGVHTFPAVLQ |
| | | | SSGLYSLSSVTVPSSSLGTQTYICNVNHKPSNTKVDKKVEPKSCD |
| | | | KTHTCPPCPAPELLGGPSVFLFPPKPKDTLMISRTPEVTCVWVDV |
| | | | SHEDPEVKFNWYVDGVEVHNAKTKPREEQYNSTYRVVSVLTVLHQ |
| | | | DWLNGKEYKCKVSNKALPAPIEKTISKAKGQPREPQVYTLPPSRD |
| | | | ELTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPG |
| | LC | 189 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEINRTVAAPSVFIFPPSDEQLKSGTASWVCL |
| | | | LNNFYPREAKVQWKVDNALQSGNSQESVTEQDSKDSTYSLSSTLT |
| | | | LSKADYEKHKVYACEVTHQGLSSPVTKSFNRGEC |
| 1-48 | HC | 191 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSAASTKGPSVFPLAP |
| | | | SSKSTSGGTAALGCLVKDYFPEPVTVSWNSGALTSGVHTFPAVLQ |
| | | | SSGLYSLSSWVTVPSSSLGTQTYICNVNHKPSNTKVDKKVEPKSC |
| | | | DKTHTCPPCPAPELLGGPDVFLFPPKPKDTLMISRTPEVTCVVDV |
| | | | SHEDPEVKFNWYVDGVEVHNAKTKPREEQYNSTYRVVSVLTVLHQ |
| | | | DWLNGKEYKCKVSNKALPLPEEKTISKAKGQPREPQVYTLPPSRD |
| | | | ELTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPG |
| | LC | 189 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEINRTVAAPSVFIFPPSDEQLKSGTASWCLL |
| | | | NNFYPREAKVQWKVDNALQSGNSQESVTEQDSKDSTYSLSSTLTL |
| | | | SKADYEKHKVYACEVTHQGLSSPVTKSFNRGEC |
| 1-49 | HC | 192 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSAASTKGPSVFPLAP |
| | | | SSKSTSGGTAALGCLVKDYFPEPVTVSWNSGALTSGVHTFPAVLQ |
| | | | SSGLYSLSSWTVPSSSLGTQTYICNVNHKPSNTKVDKKVEPKSCD |
| | | | KTHTCPPCPAPELVGGPSVFLLPPKPKDTLMISRTPEVTCVWDVS |
| | | | HEDPEVKFNWYVDGVEVHNAKTKPPEEQYNSTLRVVSVLTVLHQD |
| | | | WLNGKEYKCKVSNKALPAPIEKTISKAKGQPREPQVYTLPPSRDE |
| | | | LTKNQVSLTCLVKGFYPSDIAVEWESNGQPENNYKTTPLVLDSDG |
| | | | SFFLYSKLTVDKSRWQQGNVFSCSVMHEALHNHYTQKSLSLSPG |
| | LC | 189 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEINRTVAAPSVFIFPPSDEQLKSGTASWVCL |
| | | | LNNFYPREAKVQWKVDNALQSGNSQESVTEQDSKDSTYSLSSTLT |
| | | | LSKADYEKHKVYACEVTHQGLSSPVTKSFNRGEC |
| 1-50 | HC | 193 | QVQLVETGGGLVRPGNSLKLSCVTSGFTFSNYRMHWLRQPPGKRL |
| | | | EWIAVIKVKSDNYGANYAESVKGRFTISRDDSKSSVYLQVNRLRE |
| | | | EDTATYYCSSPTYPGSSGFAYWGQGTLVTVSAASTKGPSVFPLAP |
| | | | SSKSTSGGTAALGCLVKDYFPEPVTVSWNSGALTSGVHTFPAVLQ |
| | | | SSGLYSLSSWTVPSSSLGTQTYICNVNHKPSNTKVDKKVEPKSCD |
| | | | KTHTCPPCPAPELVGGPSVFLLPPKPKDTLMISRTPEVTCVWVDV |
| | | | SHEDPEVKFNWYVDGVEVHNAKTKPPEEQYNSTLRVVSVLTVLHQ |
| | | | DWLNGKEYKCKVSNWALPAPISKTISKAKGQPREPQVYTLPPSRD |
| | | | ELTKNQVSLTCLVKGFYPSDIAVEWESNGQPENNYKTTPLVLDSD |
| | | | GSFFLYSKLTVDKSRWQQGNVFSCSVMHEALHNHYTQKSLSLSPG |
| | LC | 189 | DIQMTQTTSSLSASLGDRVTISCRASQDISNYLNWYQQKPDGTVK |
| | | | LLIYYTSRLHSGVPSRFSGSGSGTDYSLTISNLEQEDIATYFCQQ |
| | | | GNKFPPTFGGGTKLEINRTVAAPSVFIFPPSDEQLKSGTASWVCL |
| | | | LNNFYPREAKVQWKVDNALQSGNSQESVTEQDSKDSTYSLSSTLT |
| | | | LSKADYEKHKVYACEVTHQGLSSPVTKSFNRGEC |

### Example 7: Generation of CCR8 Transfected CHO Cell Lines

The CHO-K1 cells (ATCC CCL61, supplied by the European Collection of Authenticated Cell Cultures (ECACC), ECACC No: 85051005) were transfected with plasmids pcDNA3.1 (+) containing sequences coding for the protein human CCR8 (hCCR8_P51685). Those sequences, optimized for an expression in human cells, were synthetized by TOPGene Technologies (Quebec, Canada). The CHOK1 cells, maintained in culture medium (HAM's F-12 Nutrient mix+10% Foetal Calf Serum+1% Penicillin/Streptomycin), were plated 24 h before the transfection (100 000 cells per well in a 6 well plate). Cells were transfected using Lipofectamine 2000 (Invitrogen, Villebon-sur-yvette, France) with 4 μg DNA in 250 μl OptiMEM and 10 μl of Lipofectamine according to the supplier's recommendations. Cell cultures were performed in the presence of 500 μg/ml G418 (Gibco, Villebon-sur-yvette, France). G418 resistant cell colonies were expanded and then stable CCR8 positive cells were sorted on a cell sorter (MACSQuant Tyto Cell Sorter; Miltenyi, Gladbach, Germany).

CCR8 expression was detected by flow cytometry using CCR8 antibodies for hCCR8 (anti-human CCR8 mAb PE conjugated, Clone L263G8; BioLegend, Amsterdam, The Netherlands).

FIG. 1.1 shows the cellular reactivity of the reference anti-CCR8 mAb L263G8 on hCCR8 transfected CHO cells.

### Example 8: HUT78, Cutaneous T Cell Lymphoma Cell Line

The established human HUT78 cell line available from MERCK (Sigma, St Quentin Fallavier, France) was grown in RPMI1640 medium (Gibco, Invitrogen by Thermofisher Scientific, Illkirch-Graffenstaden, France) supplemented with 10% heat-inactivated fetal bovine serum (FBS) (Gibco, Invitrogen by Thermofisher Scientific, Illkirch-Graffenstaden, France), 1% penicillin-streptomycin 10000 U/mL (Gibco, Invitrogen by Thermofisher Scientific, Illkirch-Graffenstaden, France). FIG. 1.2 shows the cellular reactivity of the reference anti-CCR8 mAb L263G8 on native hCCR8 from HUT78.

### Example 9: Human Activated T Cells

After PBMC isolation from Buffy Coat (Lymphoprep, Stemcell, Saint-Egreve, France) using standard procedure, isolation of T cells was performed using CD4+CD25+ Regulatory T cell isolation kit according to supplier recommendation (Miltenyi, Gladbach, Germany). Cells were then grown in RPMI1640 (Gibco, Invitrogen, Illkirch-Graffenstaden, France) medium supplemented with 10% heat-inactivated fetal bovine serum (FBS) (Gibco, Invitrogen, Illkirch-Graffenstaden, France), 1% penicillin-streptomycin 10000 U/mL (Gibco, Invitrogen, Illkirch-Graffenstaden, France), 500 IU/mL IL2 (Miltenyi, Gladbach, Germany) and activated using Treg expansion kit (Miltenyi, Gladbach, Germany). Cells were split in half every 2 to 3 days with fresh media and reactivated with Treg expansion kit at Day 9. Cells can be used from day 7 to day 23 post-isolation. Alternatively, activated T cells can be frozen at day 20/23, thawed and reactivated using Treg expansion kit and same culture protocol as fresh activated T cells. In this case, cells can be used at day 7/9 post thawing for analysis.

FIG. 1.3 shows the cellular reactivity of the reference anti-CCR8 mAb L263G8 on native hCCR8 from human activated T cells.

### Example 10: Analysis of mAb Specificity by Using CCR8 Coated Peptides by ELISA

The specific binding properties of antibodies were evaluated by ELISA with coated CCR8 related peptides (500 ng/ml) in carbonate/bicarbonate buffer. Different peptides were used as described in Table 1 (see Example 1). Each anti-CCR8 mAb was tested at 1 μg/mL then diluted at 1/10. After an incubation at room temperature during 90 min, unbound mAbs were removed by washings. Then the primary bound mAbs were revealed by using a goat polyclonal F(ab′)₂ fragment anti-mouse IgG, Fcγ fragment HRP Peroxidase-AffiniPure (Jackson Lab/Ozyme, Saint Cyr l'Ecole, France), following an incubation of 45 min at room temperature. After washings, 3,3′,5,5′-tetramethylbenzidine (TMB) (Interchim, Montluçon, France) was added for 15 min. Then the optical density (OD) was determined by using a multiplate reader (Tecan, Lyon, France) and correlated with mAb reactivity on the panel of coated peptides.

FIG. 2 shows the evaluation of mAb reactivity on the CCR8 related peptides pm3, pm5, pm6 and pm8 (see Table 1 above) analyzed by ELISA (mean+/−SD on two independent experiments). FIG. 3 furthermore illustrates the reactivity of exemplary mAbs on the complete panel of CCR8 related peptides. The results illustrate different mAb reactivity profiles according to their ability to bind the different sulfated tyrosine modified peptides.

### Example 11: Flow Cytometry Experiments for CCR8 Cellular Expression

The cellular mAb reactivity of the antibodies according to the present invention is tested on different CCR8 positive cells, such as the human CCR8 transfected CHO cell line versus wild type CHO cell line, the cutaneous T cell lymphoma HUT78 cell line, human activated T cell model and among CD3 positive lymphocytes from ascites in ovarian cancers (provided by Explicyte, Bordeaux, France).

The same staining protocol was applied on human CCR8 transfected CHO cell line versus wild type CHO cell line, cutaneous T cell lymphoma HUT78 cell line and human activated T cell model.

Briefly, 1.10⁵ cells per well in a 96 well plate were incubated with a dilution of unconjugated anti-CCR8 mAb at 10 μg/mL then diluted at 1/3.33, 4 dilution points (during 30 min at 4° C.). In human activated T cells model, prior to mAb incubation, cells were first incubated with 200 nM of hCCL1 (R&D Systems, Minneapolis, MN, USA) or buffer PBS (Invitrogen by Thermofisher Scientific, Illkirch-Graffenstaden, France) supplemented by 2% Fetal Bovine Serum (Invitrogen by Thermofisher Scientific, Illkirch-Graffenstaden, France) and 2 mM EDTA (Invitrogen by Thermofisher Scientific, Illkirch-Graffenstaden, France) during 1 hour at 4° C. To determine EC50 value, 8 concentrations of anti-CCR8 mAb were tested. Unbound antibodies were washed away with PBS (Invitrogen by Thermofisher Scientific, Illkirch-Graffenstaden, France) supplemented by 2% Fetal Bovine Serum (Invitrogen by Thermofisher Scientific, Illkirch-Graffenstaden, France) and 2 mM EDTA (Invitrogen by Thermofisher Scientific, Illkirch-Graffenstaden, France). Subsequently, cells are centrifuged (5 min at 400 g) and bound antibody is detected with F(ab′)2-Goat anti-Mouse IgG (H+L) Cross-Adsorbed Secondary Antibody, Alexa Fluor 488 (dilution 1/500) (Invitrogen by Thermofisher Scientific, Illkirch-Graffenstaden, France) or with F(ab′)₂-Goat anti-Human IgG Fc Secondary Antibody, PE (Invitrogen by Thermofisher Scientific, Illkirch-Graffenstaden, France) (dilution 1/1000) at 4° C. for 30 min. Detection reagent is washed away and cells are centrifuged (5 min at 400 g) and resuspended in 30 μL PBS. Bound detection antibody is quantified on a MacsQuant16 (Miltenyi, Gladbach, Germany) (B1 or B2 channel, 5000 events per acquisition). During the experiment, the respective isotype mAb controls (mouse IgG1, mouse IgG2a, mouse IgG2b, mouse IgG3 or human IgG1) (Biolegend, Amsterdam, The Netherlands) are included to exclude any unspecific binding events. EC50 values were determined via nonlinear fit “log (agonist) vs.response—Variable slope (four parameters)” function of Graphpad prism. EC50 values were not constantly determined in the same experiments.

The phenotype of human activated T cells model (CD45+,CD4+,CD25+,CD127^{low}) was evaluated before each experiment using a panel of antibodies. Representative phenotype of human activated T cells is shown in FIG. 4. After blocking Fc Receptors (FcR blocking Reagent, Miltenyi, Gladbach, Germany), 1.10⁵ cells per 96 wells are incubated with a panel of antibodies: Vioblue anti-human CD45, APC-Vio770 anti-human CD4, APC-anti human CD25, PE-Vio615 anti-human CD127 (all from Miltenyi, Gladbach Germany) in 100 UL FACS Buffer (PBS supplemented by 2% Fetal Bovine Serum and 2 mM EDTA (30 min, 4° C.)). Unbound antibodies were washed away using FACS Buffer and cells were centrifuged (5 min at 400 g) and resuspended in 50 UL of FACS Buffer. Immunophenotyping of cells is analyzed using a MacsQuant16 or MACSQuant X (Miltenyi, Gladbach, France). During the experiment, FMOI of CD127 and CD25 are performed to discriminate negative from positive cells. All antibodies used in these experiments are detailed in Table 3.

TABLE 3
List of antibodies used for flow cytometry

| Target | Clone | Fluorophore | Provider | Dilution |
|---|---|---|---|---|
| CCR8 | L263G8 | PE | Biolegend | 1/100 |
| CD3 | REA613 | Vioblue | Miltenyi | 1/50 |
| CD4 | REA623 | APC-Vio770 | Miltenyi | 1/200 |
| CD8 | REA734 | PerCP-Vio700 | Miltenyi | 1/400 |
| CD11b | MEM-174 | FITC | EXBIO | 1/10 |
| CD14 | REA599 | APC | Miltenyi | 1/400 |
| CD16 | REA423 | VioBrightR720 | Miltenyi | 1/100 |
| CD19 | REA675 | Viobright B515 | Miltenyi | 1/200 |
| CD25 | REA570 | APC | Miltenyi | 1/100 |
| CD45 | REA747 | Viogreen | Miltenyi | 1/200 |
| CD45 | REA747 | Vioblue | Miltenyi | 1/800 |
| CD56 | REA196 | PE-Vio615 | Miltenyi | 1/400 |
| CD127 | REA614 | PE-Vio615 | Miltenyi | 1/100 |

### Anti-CCR8 mAb Binding was First Characterized on Human CCR8 Transfected CHO Cell Line.

Table 4.1 shows mAb cellular reactivity (% of labeled cells and Mean Fluorescence Intensity fold change to isotype control) on hCCR8 transfected CHO cells analyzed by flow cytometry. The results showed that all the mAb panel of the invention selectively stained the human CCR8 transfected CHO cell line without any significant staining with the wild-type CHO cell line.

TABLE 4.1
mAb cellular reactivity (% of labeled cells and Mean Fluorescence
Intensity (MFI) fold change to isotype control) on hCCR8
transfected CHO cells analyzed by flow cytometry (representative
data from three independent experiments)

| | CHOK1 WT | | CHOK1 hCCR8 | |
|---|---|---|---|---|
| | MFI Fold change | % positive | MFI Fold change | % positive |
| Antibody | to isotype ctrl | cells | to isotype ctrl | cells |
| Ref#1 | 1.44 | 3.65 | 8.70 | 75.09 |
| L263G8 | 0.90 | 2.29 | 19.78 | 86.54 |
| 1-1 | 1.16 | 6.96 | 6.32 | 72.81 |
| 1-2 | 1.31 | 11.26 | 22.68 | 90.59 |
| 1-3 | 1.09 | 0.84 | 9.80 | 85.71 |
| 1-4 | 1.23 | 1.86 | 5.06 | 66.77 |
| 1-5 | 0.96 | 4.81 | 11.94 | 85.20 |
| 1-6 | 1.07 | 2.88 | 10.72 | 81.09 |
| 1-7 | 0.99 | 3.19 | 6.26 | 71.30 |
| 1-8 | 0.96 | 0.93 | 5.12 | 68.50 |
| 1-9 | 0.90 | 0.60 | 16.22 | 81.78 |
| 1-10 | 0.83 | 3.93 | 4.78 | 56.21 |
| 1-11 | 1.15 | 1.33 | 8.37 | 76.24 |
| 1-12 | 1.06 | 1.15 | 6.11 | 67.80 |
| 1-13 | 1.14 | 1.18 | 5.63 | 64.69 |
| 1-14 | 1.07 | 3.72 | 17.59 | 82.96 |
| 1-15 | 1.04 | 4.96 | 6.52 | 73.43 |
| 1-16 | 0.90 | 0.52 | 2.34 | 34.66 |
| 1-17 | 1.01 | 1.53 | 9.92 | 81.56 |
| 1-18 | 1.04 | 2.33 | 9.20 | 77.00 |

Table 4.2 represents EC50 values of the binding of some anti-CCR8 antibodies on the human CCR8 transfected CHO cell line. Anti-CCR8 antibodies of the invention bind to human CCR8 with an EC50 in nanomolar range.

TABLE 4.2
EC50 values of the binding of anti-CCR8 antibodies on CHOK1
hCCR8 transfected CHO cells analyzed by flow cytometry
(representative data from two independent experiments)

| Antibody | CHOK1 hCCR8 EC50 in nM |
|---|---|
| 1-19 | 0.83 |
| 1-20 | 0.29 |
| 1-21 | 0.35 |
| 1-22 | 1.21 |
| 1-23 | 0.36 |
| 1-24 | 0.36 |
| 1-25 | 0.10 |

Anti-CCR8 mAb cellular reactivity was also evaluated on native human CCR8 from HUT78 cell line by flow cytometry. In Table 4.3, Mean Fluorescence Intensity (Fold change to isotype control), the percentage of labelled cells and EC50 in nM of some anti-CCR8 antibodies are presented. These anti-CCR8 antibodies of the present invention bind to cell line HUT78, which expresses endogenously CCR8, with an EC50 in nanomolar or subnanomolar range.

TABLE 4.3
mAb cellular reactivity (% of labeled cells, Mean Fluorescence
Intensity (MFI) fold change to isotype control, EC50)
to cell line HUT78, which endogenously expresses CCR8
(representative data from two independent experiments)

HUT78

| | MFI Fold change | % of labelled | EC50 |
|---|---|---|---|
| Antibody | to isotype ctrl | cells | (nM) |
| 1-20 | 2.4 | 23 | 0.53 |
| 1-21 | 2.5 | 32 | 0.35 |
| 1-22 | 11.6 | 97 | 1.09 |
| 1-23 | 60.5 | 96 | 0.37 |
| 1-24 | 34.7 | 88 | 0.20 |
| 1-25 | 22.8 | 93 | 10.3 |
| 1-26 | 2.5 | 34 | 0.40 |

Binding characteristics of anti-CCR8 mAb cellular reactivity was further investigated by testing their binding to activated T cells. As shown in Table 4.4, we confirmed the binding of several exemplary anti-CCR8 antibodies of the invention on human activated T cells (MFI fold change to isotype ctrl >1). The EC50 value of the binding of anti-CCR8 antibodies was between 7 nM and 34 nM. For some anti-CCR8 antibodies, their binding on hCCR8 from human activated T cells was tested after a pre-saturation with 200 nM of hCCL1.

TABLE 4.4
mAb cellular reactivity (% of labeled cells, Mean Fluorescence Intensity (MFI)
fold change to isotype control, EC50) to human activated T cells which endogenously
express CCR8 (representative data from two independent experiments)

| | | | | Human activated T cells | | |
|---|---|---|---|---|---|---|
| | Human activated T cells | | | Post-CCL1 binding | | |
| | MFI Fold change | % of labelled | EC50 | MFI Fold change | % of labelled | EC50 |
| Antibody | to isotype ctrl | cells | (nM) | to isotype ctrl | cells | (nM) |
| 1-20 | 21 | 79 | 18.5 | 8.5 | 64 | 31.6 |
| 1-22 | 83.8 | 92 | 6.6 | 90.7 | 92 | 8.9 |
| 1-25 | 11 | 63 | 24.4 | 6.2 | 54 | 34.2 |

Illustration of the capacities of some mAbs to bind CCR8 on ovarian cancer ascite-derived immune cells is shown in FIG. 5. Scatter plot illustrations of mAb staining with the anti-CCR8 unconjugated antibody (Biolegend #360602) or with mAb 1-3 or 1-9 are given as examples.

### Example 12: Anti-CCR8 Antibodies Reactivity on Peripheral Blood Mononuclear Cells from Healthy Donors

The binding of anti-CCR8 antibodies on Peripheral Blood Mononuclear Cells from healthy donors was assessed within a multiplex panel that allows the discrimination of different immune cells by flow cytometry.

After blocking Fc Receptors (Human BD Fc Block, BD Pharmingen, Eysins, Switzerland), 5.10⁵ cells per 96 wells are incubated with a dilution of unconjugated anti-CCR8 mAb starting from 5 μg/mL, 6 dilution points (30 min at 4° C.). Rituximab, an anti-CD20 antibody, was used as control since it is known to bind exclusively on B cells. Subsequently, cells are centrifuged (5 min at 400 g) and bound antibody is detected with Goat anti-Human IgG Fc Secondary Antibody, PE (Invitrogen by Thermofisher Scientific, Illkirch-Graffenstaden, France) (dilution 1/1000) at 4° C. for 30 min. Detection reagent is washed away, cells are centrifuged (5 min at 400 g) and incubated with a panel of antibodies (CD45, CD3, CD4, CD8, CD14, CD16, CD19, CD56) (Miltenyi, Gladbach, Germany) during 30 minutes at 4° C. Two washing steps are performed, cells are resuspended in 50 μL of FACS buffer before being analyzed using a MACSQuant 16 (Miltenyi, Gladbach, Germany).

All antibodies used in these experiments are detailed in Table 3.

The gating strategy allowing the discrimination of immune cells that compose Peripheral Blood Mononuclear Cells is presented in FIG. 6.1. To identify the major circulating blood cell types, CD45 was used to target all leukocytes. Monocytes were discriminated based on their CD14 expression and then further divided into classical, intermediate, and non-classical monocytes via CD16. Among the lymphocytes gates, B cells were defined as CD3−CD19+ and T cells as CD3+CD19−. The T cells were divided into CD4+ and CD8+ T cells. CD3 and CD56 were used to distinguish CD56+NK cells, and a CD3+CD56+ NKT cell population. Among CD56+NK cells, CD56+CD16+ were gated. The percentage of binding of some anti-CCR8 antibodies of the invention within peripheral immune cells is presented in FIG. 6.2 (mean of percentage of binding from 2 independent donors). Contrary to Ref #1 that presents a binding on different peripheral immune cells, anti-CCR8 antibodies 1-20, 1-21 and 1-23 bind to less than 5% of each type of peripheral immune cells tested. In parallel to these experiments, the binding of anti-CCR8 mAb was confirmed on hCCR8 from HUT78.

### Example 13: MAb Impact on CCL1 Binding on CCR8

### Example 13.1: MAb Impact on CCL1 Binding on hCCR8 Transfected CHO Cell Line by Flow Cytometry

The hCCR8 transfected CHO cell line was dispatched at a density of 1.105 per well in a 96-well plate. Cells were incubated for 30 min at 4° C. with or without anti-CCR8 mAb tested at 10 μg/mL then diluted at 1/3. Unbound antibodies were washed away with PBS (Gibco, Villebon-sur-yvette, France) supplemented by 2% Fetal Bovine Serum (Invitrogen by Thermofisher Scientific, Illkirch-Graffenstaden, France) and 2 mM EDTA. Subsequently, cells were incubated with the recombinant human CCL1-AF647 (10 nM) (Almac, Edinburgh, Scotland) for 60 min at 4° C. After washing unbound ligands, CCL1 bound detection is quantified on a MacsQuant16 (Miltenyi, Gladbach, Germany) (R1 channel, 5 000 events per acquisition). The percentage inhibition of hCCL1-AF647 binding in the presence of tested mAb was calculated as follows:

$$1 - ( \frac{MF exp - MFInc}{MFIpc - MFInc} ) * 100$$

where MFI_{PC} of MFI of the positive control, i.e. MFI observed in the presence of CCL1-AF647 alone, MFIₑₓₚ is MFI of CCL1-AF647 observed after incubation with the mAb tested, and MFI_{NC} is MFI of the negative control, i.e. MFI observed by the binding of hCCL1-AF647 on a CCR8 negative cell line. For IC₅₀ determination, a concentration response curve is performed using 8 concentrations of mAb. Curves are fitted and IC₅₀ of CCL1 binding blocking activity is calculated using the sigmoidal dose-response (variable slope) model on GraphPad Prism 9 (GraphPad Software, San Diego, CA, USA).

FIG. 7 and Table 5.1 illustrate the evaluation of the anti-CCR8 mAb panel of the present invention for their potential to functionally inhibit the binding of CCR8 ligand CCL1 on CCR8 expressed at the cell surface analyzed by flow cytometry on hCCR8 transfected CHO cell line (mean of two independent experiments). Different mAb activity can be observed according to the level of CCL1 inhibition. Some of them completely prevented CCL1 binding, with IC₅₀ range around 1-20 nM.

TABLE 5.1
IC₅₀ values of anti-CCR8 mAbs for inhibiting the binding of
CCL1 to CCR8, as analyzed by flow cytometry on hCCR8 transfected
CHO cell line. Mean of two independent experiments.

| Antibody | IC₅₀ (μg/mL) | IC₅₀ (nM) |
|---|---|---|
| Ref#1 | 0.65 | 4.19 |
| L263G8 | 0.40 | 2.70 |
| 1-1 | 1.02 | 6.83 |
| 1-2 | 0.83 | 5.55 |
| 1-3 | 1.29 | 8.62 |
| 1-4 | 1.93 | 12.95 |
| 1-5 | 2.65 | 15.18 |
| 1-6 | 1.30 | 8.71 |
| 1-7 | 0.89 | 5.70 |
| 1-8 | 2.55 | 17.05 |
| 1-9 | 0.09 | 6.09 |
| 1-10 | 0.06 | 4.01 |
| 1-11 | 0.72 | 4.82 |
| 1-12 | 0.85 | 5.72 |
| 1-13 | 0.36 | 2.42 |
| 1-14 | 0.28 | 1.85 |
| 1-15 | 0.75 | 5.01 |
| 1-16 | 6.41 | 42.95 |
| 1-17 | 1.18 | 7.92 |
| 1-18 | 0.22 | 1.46 |

### Example 13.2: MAb Impact on CCL1 Binding on hCCR8 (HTRF)

The inhibition of CCL1 binding to hCCR8 was evaluated with the Tag-Lite® technology from Cisbio (Revvity). Human CCR8 is engineered with a SNAP-tag at the N-terminus of the GPCR. hCCR8 SNAP-tag is transiently transfected on HEK-293 cell line using FuGene® (Promega, Madison, WI, USA). 48 hours after transfection, cells are labeled with the substrate Lumi4-Terbium® (Cisbio, Codolet, France) which covalently reacts with the SNAP-tag, for 1 h at 37° C. Thawed or freshly labeled cells are plated in a 384 low volume white plate (GREINER Bio-One, Courtaboeuf, France) (10000 cells/well/10 μl). Subsequently, 5 μl of the mAbs (10, 1, 0.1, 0.01, 0.001, 0.0001 μg/ml) to be tested are added, followed by 5 μl of hCCL1-AF647 (Almac CAF-07, Edinburgh, Scotland) at the concentration of the Kd. The Kd was previously obtained in a saturation binding assay on the same cells. The non-specific binding is evaluated by adding at the same concentration of hCCL1-AF647 at least 100-fold molar excess of unlabeled hCCL1 (R&D Systems, Minneapolis, MN, USA). The plate is next incubated at 21° C. for 2 h. Detection of HTRF signal is performed on the multiplate reader EnVision (PerkinElmer, Waltham, MA, USA). The HTRF (665/620) ratio is calculated by EnVision software, the data exported are considered as the total binding. The specific binding is calculated then by subtracting the non-specific signal from the total binding. The Ki and IC₅₀ are calculated using the “One site-Fit Ki” and “One site-FitlogIC50” from GraphPad Prism 9 (Graphpad Software, San Diego, CA, USA), respectively.

Table 5.2 illustrates the evaluation of the anti-CCR8 mAb panel of the present invention for their potential to inhibit CCL1 binding on hCCR8 expressed at the cell surface, analyzed by HTRF on hCCR8 transfected into HEK-239 cell line (mean of two independent experiments). Anti-CCR8 mAb of invention inhibit CCL1 binding with IC₅₀ range between 0.1 and 1 nM.

TABLE 5.2
IC₅₀ and Ki values of anti-CCR8 mAbs for inhibiting
the binding of CCL1 to CCR8, as analyzed by HTRF
on hCCR8 transiently transfected HEK-293 cell
line. Mean of two independent experiments.

| Antibody | IC₅₀ (μg/mL) | IC₅₀ (nM) | Ki (μg/mL) | Ki (nM) |
|---|---|---|---|---|
| Ref#1 | 0.45 | 3.02 | 0.23 | 1.51 |
| L263G8 | 0.14 | 0.96 | 0.07 | 0.48 |
| 1-19 | 0.02 | 0.12 | 0.01 | 0.06 |
| 1-20 | 0.09 | 0.63 | 0.05 | 0.31 |
| 1-21 | 0.09 | 0.60 | 0.05 | 0.30 |
| 1-22 | 0.15 | 0.99 | 0.08 | 0.50 |
| 1-23 | 0.04 | 0.23 | 0.02 | 0.12 |
| 1-24 | 0.04 | 0.24 | 0.02 | 0.12 |
| 1-25 | 0.02 | 0.14 | 0.01 | 0.07 |

### Example 14: Functional Characterization of CCR8 Binders on CCL1 Induced Gi2 Signaling Pathways from Human CCR8

The assay used to measure mAb activity is based on bioSensAll®, a live-cell bioluminescence resonance energy transfer (BRET)-based biosensor platform. The technology monitors the plasma membrane recruitment of proteins that interact specifically with the active a subunit of a G protein (Avet C et al., Elife, 2022, 11: e74101, doi: 10.7554/eLife.74101). HEK-293 cells (Namkung Y et al., Nat Commun, 2016, 7:12178, doi: 10.1038/ncomms12178) are co-transfected using polyethylenimine (25 kDa linear, Alfa Aesar, Kandel, Germany) and a transfection mix containing plasmids (synthetized by TOPGene Technologies, Quebec, Canada) coding for human CCR8, the Gαi2 protein, the energy donor (rLucII) and the energy acceptor (rGFP). After transfection, cells are cultured in flask (7.10E06 cells per Flask 175 cm²) for 48 h at 37° C./5% CO₂. The day of the assay, cells are rinsed, detached, centrifuged, and resuspended in assay buffer (HBSS 21-023-CV, Corning, St. Quentin Fallavier, France)+0.05% (w/v) BSA FFA A7030-100g (Sigma, St. Quentin Fallavier, France). For pH assay, antibodies were retested in independent experiment. The buffer used during the test is HBSS+0.05% (w/v) BSA FFA stabilized with 50 mM MES (2-(N-morpholino) ethanesulfonic acid) and adjusted at pH 6.5 or at pH 7.4 with NaOH. Cells are then seeded in white opaque 384 well plate (Greiner 781080, Sigma, St. Quentin Fallavier, France) (20,000 cells/well/20 μl). Then, plates are equilibrated 1 hour at 22° C. before mAb addition. Investigation of agonist or antagonist mAb activity was measured in the same well. Antibodies prepared 4-fold concentrated in assay buffer are incubated with cells (10 μl/well), 1 h at room temperature. First, cells are incubated with mAbs for 1 hour, and then hCCL1 is added to the wells. The luciferase substrate (Prolume Purple, Nanolight Technology, US) is then added (10 μl/well, 2 μM final concentration) and the agonist activity is immediately detected. BRET readings are collected on Spark (TECAN, Lyon, France) with specific filters (415-455 nm for the Rluc, 505-545 nm for the GFP). Luminescence is recorded for additional 21 minutes to measure antagonist effect. These steps are common for the three protocols described below.

### Protocol #1

Cells are stimulated with 6 nM hCCL1 (R&D Systems, Minneapolis, MN, USA) corresponding to the concentration inducing 80% of the maximal agonist effect (EC80) for antagonist effect, which is added to the well using the automated device D300E (TECAN, Lyon France). For IC₅₀ determination, a concentration response curve is performed in duplicates in 2 independent experiments, using 8 concentrations (ranging over 7 logs) of mAb. Curves are fitted and IC₅₀ of antagonist activity is calculated using the sigmoidal dose-response (variable slope) model on GraphPad Prism 9 (GraphPad Software, San Diego, CA, USA).

### Protocol #2

A protocol similar to protocol #1 was used for a second series of experiments aiming at determining the insurmountable nature of the antagonist mAbs with a higher concentration of 100 nM hCCL1. The experiment is performed in duplicates. The residual responses of 100 nM hCCL1 were calculated as a percentage using the buffer response as 0% and 100 nM hCCL1 response as 100%. Comparison between the residual responses induced by 100 nM hCCL1 concentration in the absence or presence of the mAb informs about its insurmountable character.

### Protocol #3

In addition, a third protocol is also deployed to measure the potency of insurmountable mAbs, which corresponds to the Gi2 BRET assay based on the shift of a hCCL1 concentration-response curve in the presence of a mAb concentration. The use of the Schild analysis is described in the book “A pharmacology Primer”, 5th Edition, 2021, from Kenakin. The residual responses of 100 nM hCCL1 were calculated as a percentage using the buffer response as 0% and 100 nM hCCL1 response as 100%. A comparison between the responses induced by the highest hCCL1 concentration in absence or presence of the mAb informs about its insurmountable character. Curves representing residual response are fitted using the sigmoidal dose-response (3 parameter) model on GraphPad Prism 9 (GraphPad Software, San Diego, CA, USA). The equation used, called a three-parameter dose-response curve, assumes that the dose response curves have a standard slope, equal to a Hill slope (or slope factor) of 1.0, with a top and bottom which are plateaus in the units of the Y axis. Concentration-response experiments are performed in duplicate, in two independent experiments. In the case of a surmountable mAb antagonist, the top plateau value is expected to be around 100%. If the calculated value of the top plateau is greater than 100%, it reflects an insurmountable nature of the mAb antagonist.

### Protocol #4

The duration of antagonism of CCR8 inhibitors was evaluated using “wash-out” studies. The test was developed on adherent cells in order to remove pre-incubated mAbs. After transfection, cells are seeded in plate (10,000 cells per well in 384-well plate) for 48 h at 37° C./5% CO₂. The day of the test the medium is discarded using the Blue®Washer (BlueCatBio GmbH) and 20 UL of buffer is added. Then antibodies prepared 4-fold concentrated in assay buffer are incubated with cells (10 μl/well), 1 h at 22° C. For the wash-out condition, mAbs are discarded (using the Blue®Washer (BlueCatBio GmbH) and buffer (30 μL/well) is added. The plate is then incubated 6 h at 22° C. At 0 (No wash-out) and 6 h (Wash-out) post wash-out, the luciferase substrate (Prolume Purple, Nanolight Technology, US) is added (10 μL/well, 2 μM final concentration) and to determine antagonist effect cells are stimulated with 0.6 nM hCCL1 (R&D Systems, Minneapolis, MN, USA) corresponding to the concentration inducing 80% of the maximal agonist effect (EC80), which is added to the well using the automated device D300E (TECAN, Lyon France). BRET readings are collected on Spark (TECAN, Lyon, France) with specific filters (415-455 nm for the Rluc, 505-545 nm for the GFP). Luminescence is recorded for additional 21 minutes to measure antagonist effect.

A concentration response curve is performed in duplicates in 2 independent experiments, using 8 concentrations (ranging over 7 logs) of mAb. Curves are fitted and IC₅₀ of antagonist activity is calculated using the sigmoidal dose-response (variable slope) model on GraphPad Prism 9 (GraphPad Software, San Diego, CA, USA).

FIG. 8 and Table 6 illustrate the evaluation of the anti-CCR8 mAb panel of the present invention for their potential to functionally inhibit CCL1 induced hCCR8 Gi2 signaling pathway analyzed by BRET on transiently transfected HEK-293 cell line (mean+/−SD on two independent experiments). All of the tested antibodies of the present invention completely prevented CCL1 induced hCCR8 Gi2 signaling pathway, with IC₅₀ ranging from 0.004 μg/mL to 0.15 μg/mL. None of these mAbs alone triggered the hCCR8 Gi2 signaling pathway (data not shown).

TABLE 6
IC₅₀ of anti-CCR8 mAbs for inhibiting CCL1 induced hCCR8 related
Gi2 signaling (as determined using BRET from hCCR8 transfected
HEK-293 cell line). Mean of two independent experiments.

| Antibody | IC₅₀ (μg/mL) | IC₅₀ (nM) |
|---|---|---|
| Ref#1 | 0.101 | 0.675 |
| L263G8 | 0.004 | 0.026 |
| 1-1 | 0.023 | 0.153 |
| 1-2 | 0.029 | 0.191 |
| 1-3 | 0.069 | 0.462 |
| 1-4 | 0.034 | 0.227 |
| 1-5 | 0.143 | 0.954 |
| 1-6 | 0.049 | 0.328 |
| 1-7 | 0.018 | 0.117 |
| 1-8 | 0.031 | 0.209 |
| 1-9 | 0.015 | 0.1 |
| 1-10 | 0.046 | 0.306 |
| 1-11 | 0.129 | 0.86 |
| 1-12 | 0.015 | 0.099 |
| 1-13 | 0.01 | 0.067 |
| 1-14 | 0.01 | 0.066 |
| 1-15 | 0.019 | 0.129 |
| 1-16 | 0.092 | 0.616 |
| 1-17 | 0.026 | 0.172 |
| 1-18 | 0.004 | 0.029 |
| 1-19 | 0.048 | 0.318 |
| 1-20 | 0.029 | 0.194 |
| 1-21 | 0.035 | 0.232 |
| 1-22 | 0.068 | 0.452 |
| 1-23 | 0.022 | 0.148 |
| 1-24 | 0.034 | 0.224 |
| 1-25 | 0.02 | 0.134 |

Table 7 illustrates the residual responses of 100 nM hCCL1 in both protocol #2 and protocol #3. Values are given in presence of 10 μg/mL/0.4 μg/mL/0.08 μg/mL of mAbs for protocol #2, and in presence of 5 μg/ml/0.5 g/mL/0.05 μg/mL of mAbs for protocol #3. As can be observed, all the tested antibodies of the present invention depressed the maximal response induced by hCCL1 at 100 nM indicating an insurmountable nature of their antagonistic activity.

TABLE 7
hCCL1 (100 nM) residual response in %

| | Protocol #2 | | | Protocol #3 | | |
|---|---|---|---|---|---|---|
| | mAb concentration | | | mAb concentration | | |
| | 10 μg/mL | 0.4 μg/mL | 0.08 μg/mL | 5 μg/mL | 0.5 μg/mL | 0.05 μg/mL |
| | 66.7 nM | 2.67 nM | 0.53 nM | 33.3 nM | 3.33 nM | 0.33 nM |
| 1-1 | 44 | 69 | 81 | 49 | 57 | 77 |
| 1-2 | 19 | 36 | 60 | 48 | 68 | 74 |
| 1-3 | 100 | 93 | 101 | 73 | 81 | 92 |
| 1-4 | 28 | 49 | 72 | 22 | 36 | 75 |
| 1-5 | 97 | 97 | 99 | 75 | 84 | 99 |
| 1-6 | 17 | 38 | 79 | 47 | 64 | 102 |
| 1-7 | 24 | 51 | 73 | 30 | 38 | 69 |
| 1-8 | 51 | 44 | 70 | 43 | 55 | 78 |
| 1-9 | 46 | 57 | 88 | 36 | 36 | 73 |
| 1-10 | 28 | 46 | 104 | 45 | 57 | 80 |
| 1-11 | * | * | * | 84 | 98 | 102 |
| 1-12 | 68 | 85 | 99 | 48 | 75 | 86 |
| 1-13 | 21 | 35 | 60 | 38 | 39 | 69 |
| 1-14 | 48 | 89 | 93 | 50 | 68 | 92 |
| 1-15 | 95 | 102 | 99 | 67 | 97 | 102 |
| 1-16 | 84 | 96 | 97 | 73 | 81 | 96 |
| 1-17 | 56 | 83 | 99 | 45 | 90 | 116 |
| 1-18 | 53 | 68 | 52 | 59 | 62 | 76 |
| 1-20 | 30 | 38 | 62 | 39 | 63 | 94 |
| 1-21 | 40 | 54 | 74 | 51 | 63 | 89 |
| 1-22 | 31 | 77 | 94 | 33 | 76 | 110 |
| 1-23 | 32 | 55 | 67 | 42 | 60 | 86 |
| 1-24 | 41 | 66 | 77 | 37 | 52 | 73 |

(*: Not done)

Table 8 illustrates the calculated (using the sigmoidal dose-response (3 parameter) model on GraphPad Priem 9) top plateau values from the protocol #3 where hCCL1 concentration-responses were tested against a fix concentration of mAbs (5 g/mL). As can be observed, these top plateau values are above 100% for all tested antibodies of the present invention indicating an insurmountable nature of their antagonistic activity.

TABLE 8
Top plateau values extrapolated by GraphPad
Prism (% of hCCL1 (100 nM))

| Antibody | Top plateau values in presence of 5 μg/mL mAb |
|---|---|
| 1-1 | 759653 |
| 1-2 | 640153 |
| 1-3 | 663 |
| 1-4 | 313030 |
| 1-5 | 133 |
| 1-6 | 667021 |
| 1-7 | 526067 |
| 1-8 | 511361 |
| 1-9 | 532752 |
| 1-10 | 855 |
| 1-11 | 2880 |
| 1-12 | 804588 |
| 1-13 | 149 |
| 1-14 | 304570 |
| 1-15 | 134935 |
| 1-16 | 292 |
| 1-17 | 192911 |
| 1-18 | 948 |
| 1-20 | 517389 |
| 1-21 | 137660 |
| 1-22 | 76423 |
| 1-23 | 569402 |
| 1-24 | 365683 |

The insurmountable antagonist effect of several exemplary antibodies according to the invention on CCL1-induced CCR8-related Gi2 signaling is furthermore illustrated in FIG. 10.

The antagonist activity of the antibodies according to the invention was furthermore examined at pH 6.5 and PH 7.4. Thus, using the above-described pH assay according to protocol #1, several exemplary anti-CCR8 mAbs were evaluated for their potential to functionally inhibit CCL1 induced hCCR8 Gi2 signaling pathway analyzed by BRET on transiently transfected HEK-293 cell line at pH 6.5 or at pH 7.4 (mean+/−SD on two independent experiments). As reflected by the corresponding results shown in FIG. 11 and Table 9, the anti-CCR8 mAbs according to the invention were found to be potent antagonists of the CCL1-induced hCCR8 Gi2 signaling pathway, not only at a neutral pH of 7.4 but also at an acidic pH of 6.5 which is indicative of the pH in the tumor microenvironment.

TABLE 9
IC₅₀ of anti-CCR8 mAbs for inhibiting CCL1 induced hCCR8
related Gi2 signaling (as determined using BRET on hCCR8
transfected HEK cell line) at two different pH values (pH
6.5 or pH 7.4). Mean of two independent experiments.

| | |
|---|---|
| pH 6.5 | pH 7.4 |

| Antibody | IC₅₀ μg/ml | IC₅₀ NM | IC₅₀ μg/ml | IC₅₀ NM |
|---|---|---|---|---|
| 1-1 | 0.021 | 0.143 | 0.024 | 0.163 |
| 1-2 | 0.03 | 0.198 | 0.031 | 0.21 |
| 1-3 | 0.051 | 0.34 | 0.059 | 0.396 |
| 1-4 | 0.034 | 0.225 | 0.065 | 0.434 |
| 1-5 | 0.414 | 2.762 | 0.359 | 2.396 |
| 1-6 | 0.044 | 0.292 | 0.061 | 0.405 |
| 1-7 | 0.011 | 0.073 | 0.007 | 0.047 |
| 1-8 | 0.053 | 0.355 | 0.063 | 0.421 |
| 1-12 | 0.031 | 0.208 | 0.017 | 0.115 |
| 1-13 | 0.019 | 0.125 | 0.011 | 0.075 |
| 1-14 | 0.014 | 0.095 | 0.004 | 0.026 |
| 1-16 | 0.162 | 1.083 | 0.152 | 1.014 |
| 1-21 | 0.025 | 0.165 | 0.035 | 0.232 |

### Example 15: MAb Ability to Prevent CCL1-Induced Internalization of hCCR8

The assay is based on diffusion-enhanced resonance energy transfer between cell surface GPCRs labeled with a luminescent terbium cryptate donor and a fluorescein acceptor present in the culture medium (Levoye A et al., Front. Endocrinol. 6:167, doi: 10.3389/fendo.2015.00167). GPCR internalization results in a quantifiable reduction of energy transfer. Stable HEK-293 cell line expressing CCR8 SNAP-tag are seeded 24 h before the assay in a 384 low-volume well plate (GREINER Bio-One, Courtaboeuf, France) at 10,000 cells/well in 20 μl/well. The plate is placed at 37° C., 5% CO₂ for 24 h. The day of the assay, cells are labeled with SNAP-tag substrate Lumi4-Terbium® (Cisbio, Codolet, France), and incubated at 4° C. for 1 hour. Then, the substrate is washed 3 times with Tag-lite buffer 1X (Cisbio, Codolet, France). After adding 10 μl of fluorescein (Sigma, St Quentin Fallavier, France) (50 μM) to the labelled cells, the plate is incubated 5 min at 37° C., to equilibrate the temperature before the internalization monitoring. To evaluate the effect of the antibodies on the internalization induced by hCCL1 (R&D Systems, Minneapolis, MN, USA), 5 μl of antibodies (5 or 10 μg/ml) are added to the cells followed by 5 μl of hCCL1 300 nM. The different compounds are added to the plate at room temperature. The plate is then placed in the EnVision plate multireader (PerkinElmer, Waltham, MA, USA), measurements are taken every 5 min for 1 hour, then every 15 min for 2 hours at 37° C. The HTRF ratio (R) (620/520) is calculated with EnVision software. The percentages of internalization are calculated using the following formula: [(Rt−Rmin)/(Rmax−Rmin)]×100 (Rt: HTRF Ratio mAb, Rmin: HTRF Ratio buffer, Rmax: HTRF Ratio CCL1 300 nM) at the time max of CCR8 SNAP-tag internalization with CCL1 300 nM, 30-35 min. Data are analyzed using Excel and GraphPad Prism 9 (Graphpad Software, San Diego, CA, USA).

Table 10 illustrates the effect of mAbs on hCCR8 internalization induced by CCL1 (300 nM) analyzed by HTRF. The anti-CCR8 mAb panel of the present invention was found to inhibit the internalization of hCCR8 induced by CCL1 (300 nM) on HEK-293 cell line. All the antibodies were tested at 10 μg/ml and 1 μg/ml, except 1-21 that was tested at 5 μg/ml and 2 μg/ml.

TABLE 10
Effect of mAbs on hCCR8 internalization coincubated with CCL1 (300 nM). Data expressed
in percentage of hCCR8 internalization where 100% of internalization is CCL1 at
300 nM. Mean of two independent experiments (SD = standard deviation).

| | % of | | % of | | % of | | % of | |
|---|---|---|---|---|---|---|---|---|
| Antibody + | internalization | | internalization | | internalization | | internalization | |
| CCL1 | at 10 μg/ml | SD | at 5 μg/ml | SD | at 2 μg/ml | SD | at 1 μg/ml | SD |
| 1-21 | Not done | / | 46 | 7 | 57 | 7 | Not done | / |
| 1-22 | 52 | 32 | Not done | / | Not done | / | 139 | 10 |
| 1-23 | 29 | 1 | Not done | / | Not done | / | 44 | 2 |
| 1-24 | 36 | 10 | Not done | / | Not done | / | 44 | 13 |
| Ref#1 | 111 | 5 | Not done | / | Not done | / | 126 | 18 |
| Ref#2 | 90 | 3 | Not done | / | Not done | / | 100 | 19 |
| Ref#3 | 103 | 10 | Not done | / | Not done | / | 95 | 12 |

### Example 16: ADCC Assay

ADCC induced anti-CCR8 activities were assessed using HUT78 as target cells and NK cells as effector cells. PBMC were isolated from buffy coat using Ficoll-Paque™PLUSMedia (GEHealthcare) and 50 mL Leucoseptubes (GreinerBioOne) according to manufacturer's instructions. NK cells were sorted from total alive PBMCs (400-600 millions of cells) with “EasySep™ Human NK Cell Enrichment Kit” (Stemcell) according to manufacturer's instructions with RoboSep™-S(Stemcell). NK cell purity (percentage of NK cells: CD3-CD56+ cells) and expression of CD16 marker were evaluated by flow cytometry for each donor. Target cells were stained with calcein AM (Invitrogen) diluted at 10 μM (HUT-78) in RPMI medium (RPMI 1640 without phenol red (Gibco)+2% FBS+Probenecid 0.77 mg/mL (Invitrogen) according to manufacturer's instructions. Antibodies were diluted following the desired concentration in RPMI medium and incubated with target (5,000 cells) and effector cells (50,000 cells) with an Effector to Target ratio (E: T) of 10:1 in a final volume of 250 UL during 2 h at 37° C. in 5% CO₂ incubator. Supernatants are transferred to black flat-bottom 96 well plate (GreinerBioOne). Fluorescence is read with CLARIOstar spectrometer (BMGlabtech) with the settings: excitation wavelength=492 nm, emission wavelength=515 nm and bandwidth=10 nm. Determination of specific lysis %=100×(Exp release−Spontaneous release)/(Maximum release−Spontaneous release)

Exp. (experimental) release being fluorescence value obtained for target cells and effector cells in presence of tested compounds, spontaneous release (of calcein) being fluorescence value obtained with target cells in presence of effector cells, and maximum release being fluorescence value obtained for target cells and effector cells with lysis buffer.

EC50 values were determined via nonlinear fit “log (agonist) vs.response—Variable slope (four parameters)” function of Graphpad prism.

FIG. 12 shows anti-CCR8 mAb induced ADCC activities for multiple antibodies according to the invention in HUT78 expressing human CCR8 as target cells and NK cells as effector cells.

ADCC activities were characterized by maximum percentage lysis of target cells (see FIGS. 12.1 to 12.4), for all mAbs a specific lysis of target cells was observed. For some anti-CCR8 mAbs, the ADCC potency was further characterized with both maximum percentage lysis of target cells and the determination of EC50 in pM. As shown in Table 11.1 and 11.2, anti-CCR8 mAbs induced the lysis of HUT78 cell line target cells through ADCC mechanism, from 14% to 31% lysis. EC50 value for ADCC activity induced by anti-CCR8 mAbs ranging from nanomolar (1.9 nM) range to picomolar range (0.42 pM) for the more potent anti-CCR8 mAbs are presented in Table 11.1 and 11.2. Dose response curves of percentage of specific lysis ADCC are presented in FIGS. 12.1 to 12.4, for each mAb, two independent NK donors are represented.

TABLE 11.1
Anti-CCR8 mAb induced ADCC: EC50 and Max percentage
lysis using HUT78 as target cells and NK cells
(Donor 1 and Donor 2) as effector cells

| | NK cells: Donor 1 | | NK cells: Donor 2 | |
|---|---|---|---|---|
| | % Max lysis | EC50 (pM) | % Max lysis | EC50 (pM) |
| Lacutamab | 41 | 38.03 | 41 | 104.96 |
| 1-21 | 30 | 6.43 | 28 | 0.42 |
| 1-26 | 22 | 2.24 | 20 | 2.16 |
| 1-28 | 31 | 73.26 | 26 | 90.38 |

TABLE 11.2
Anti-CCR8 mAb induced ADCC: EC50 and Max percentage
lysis using HUT78 as target cells and NK cells
(Donor 3 and Donor 4) as effector cells

| | NK cells: Donor 3 | | NK cells: Donor 4 | |
|---|---|---|---|---|
| | % Max lysis | EC50 (pM) | % Max lysis | EC50 (pM) |
| Lacutamab | 40 | 3.80 | 29 | 1.71 |
| 1-23 | 15 | 504.63 | 16 | 572.56 |
| 1-33 | 14 | 792.54 | 15 | 1517.15 |
| 1-34 | 25 | 217.98 | 28 | 53.03 |
| 1-35 | 29 | 194.81 | 29 | 281.78 |
| 1-25 | 16 | 348.72 | 19 | 1906.76 |
| 1-45 | 16 | 291.38 | 18 | 405.86 |
| 1-48 | 25 | 8.22 | 30 | 48.90 |

### Example 17: ADCP Assay

ADCP induced anti-CCR8 activities were assessed using HUT78 as target cells and monocyte-derived macrophages (MDM) as effector cells.

Monocytes were purified from heparinized whole blood with monocyte enrichment cocktail using StemCell protocol. Cells were cultured at 10⁶ cells/mL in X-VIVO15 (Lonza) with 500 IU/mL of hGM-CSF (Miltenyi) for 14 days. Target cells HUT78 that express endogenously CCR8 were stained with PKH26 (Sigma) according to Sigma protocol. PKH26 labeled target cells were co-cultured with MDM (E: T Ratio 4:1) and incubated with serial dilutions of anti-CCR8 antibodies or irrelevant control (Rituximab) for 1 h at 37° C. After being washed, cells were stained with CD11b-FITC (10 μL/test) for 30 min at 4° C. Cells were analyzed by flow cytometry. The percentage of phagocytosed target cells was calculated: % of CD11b+/PKH26+ cells/(% of CD11b+/PKH26+ cells+% of CD11b−/PKH26+ cells). Rituximab is a chimeric mAb targeting CD20 and was used as negative control. Lacutamab is a humanized mAb targeting KIR3DL2 and was used as positive control.

FIG. 13 and Table 12 show ADCP activities of some anti-CCR8 antibodies according to the invention on HUT78 as target cells and MDM as effector cells. Maximum percentage of phagocytosis was determined by flow cytometry as double positive CD11b+/PKH26+ on three independent donors.

Anti-CCR8 antibodies induced the phagocytosis of HUT78 cells by monocyte-derived macrophages on two independent donors. Maximum percentage phagocytosis for 1-33 was 17.9% (Donor 5) and 27.5% (Donor 6). Maximum percentage phagocytosis for 1-34 was 20.8% (Donor 5) and 38.1% (Donor 6). The antibodies 1-21, 1-26 and 1-34 induced the phagocytosis of HUT78 (Max percentage phagocytosis from 7.27% to 10.62%) by MDM from another donor (Donor 9).

TABLE 12
Anti-CCR8 antibodies induced ADCP activities

| | Monocyte-derived | Monocyte-derived | Monocyte-derived |
|---|---|---|---|
| | macrophages - | macrophages - | macrophages - |
| | Donor 5 | Donor 6 | Donor 9 |
| Antibody | % max phagocytosis | | |
| Rituximab | 10.8 | 6.94 | 6.26 |
| Lacutamab | 29.0 | 16.1 | 10.62 |
| 1-21 | * | * | 9.65 |
| 1-26 | * | * | 8.29 |
| 1-33 | 17.9 | 27.5 | * |
| 1-34 | 20.8 | 38.1 | 7.27 |

(* Not tested)

1. A monoclonal antibody or an antigen-binding fragment thereof, wherein said antibody or antigen-binding fragment specifically binds to human CCR8 and is an insurmountable antagonist of the CCL1-CCR8 signaling pathway.

2. The antibody or antigen-binding fragment according to claim 1, wherein the antibody or antigen-binding fragment is an insurmountable antagonist of CCL1-induced CCR8-Gi₂ signaling.

3. The antibody or antigen-binding fragment according to claim 1 or 2, wherein the antibody or antigen-binding fragment inhibits the binding of human CCL1 to human CCR8 with an IC₅₀ of about 20 nM or less, preferably with an IC₅₀ of about 10 nM or less.

4. The antibody or antigen-binding fragment according to any one of claims 1 to 3, wherein the antibody or antigen-binding fragment comprises:
(1) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23);
wherein in each of said CDR-H1, said CDR-H2, said CDR-H3, said CDR-L1, said CDR-L2, and said CDR-L3, a single amino acid residue is optionally replaced by a different amino acid residue; or
(2) a heavy chain variable domain (VH) comprising a CDR-H1 having the amino acid sequence PYAMN (SEQ ID NO: 6), a CDR-H2 having the amino acid sequence RIRSKSNNYATYYADSVKD (SEQ ID NO: 7), and a CDR-H3 having the amino acid sequence GREAYYRYDGDYYAMDY (SEQ ID NO: 8); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); wherein in each of said CDR-H1, said CDR-H2, said CDR-H3, said CDR-L1, said CDR-L2, and said CDR-L3, a single amino acid residue is optionally replaced by a different amino acid residue; or
(3) a heavy chain variable domain (VH) comprising a CDR-H1 having the amino acid sequence TYAMN (SEQ ID NO: 12), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); wherein in each of said CDR-H1, said CDR-H2, said CDR-H3, said CDR-L1, said CDR-L2, and said CDR-L3, a single amino acid residue is optionally replaced by a different amino acid residue; or
(4) a heavy chain variable domain (VH) comprising a CDR-H1 having the amino acid sequence EYTIH (SEQ ID NO: 14), a CDR-H2 having the amino acid sequence GINPNGDTRYDQKFKG (SEQ ID NO: 15), and a CDR-H3 having the amino acid sequence VARFYGISPYAMDY (SEQ ID NO: 16); and
a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQSTHVPYT (SEQ ID NO: 19); wherein in each of said CDR-H1, said CDR-H2, said CDR-H3, said CDR-L1, said CDR-L2, and said CDR-L3, a single amino acid residue is optionally replaced by a different amino acid residue; or
(5) a heavy chain variable domain (VH) comprising a CDR-H1 having the amino acid sequence AYAMN (SEQ ID NO: 20), a CDR-H2 having the amino acid sequence RIRSKSNDYATYYGDSVKD (SEQ ID NO: 21), and a CDR-H3 having the amino acid sequence GREAYYRYDGGYYAMDY (SEQ ID NO: 13); and
a light chain variable domain (VL) comprising a CDR-L1 having the amino acid sequence TSSKSLLHSNGNTYLY (SEQ ID NO: 22), a CDR-L2 having the amino acid sequence RMSNLAS (SEQ ID NO: 10), and a CDR-L3 having the amino acid sequence MQHREYPFT (SEQ ID NO: 23); wherein in each of said CDR-H1, said CDR-H2, said CDR-H3, said CDR-L1, said CDR-L2, and said CDR-L3, a single amino acid residue is optionally replaced by a different amino acid residue; or
(6) a heavy chain variable domain (VH) comprising a CDR-H1 having the amino acid sequence DYVVT (SEQ ID NO: 24), a CDR-H2 having the amino acid sequence VIWGGGNTYYNSDLKS (SEQ ID NO: 25), and a CDR-H3 having the amino acid sequence RHRDYALDY (SEQ ID NO: 26); and
a light chain variable domain (VL) comprising a CDR-L1 having the amino acid sequence RSSQSLVYSNGNTYLH (SEQ ID NO: 27), a CDR-L2 having the amino acid sequence KVSNRFS (SEQ ID NO: 18), and a CDR-L3 having the amino acid sequence SQSTYVPPT (SEQ ID NO: 28); wherein in each of said CDR-H1, said CDR-H2, said CDR-H3, said CDR-L1, said CDR-L2, and said CDR-L3, a single amino acid residue is optionally replaced by a different amino acid residue; or
(7) a heavy chain variable domain (VH) comprising a CDR-H1 having the amino acid sequence DYTLH (SEQ ID NO: 29), a CDR-H2 having the amino acid sequence GITPKNGDTRYDPRFKD (SEQ ID NO: 30), and a CDR-H3 having the amino acid sequence VARFYGVSPYAMDY (SEQ ID NO: 31); and
a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQTTHVPYT (SEQ ID NO: 32); wherein in each of said CDR-H1, said CDR-H2, said CDR-H3, said CDR-L1, said CDR-L2, and said CDR-L3, a single amino acid residue is optionally replaced by a different amino acid residue; or
(8) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PDGFSPFVY (SEQ ID NO: 35); and
a light chain variable domain (VL) comprising a CDR-L1 having the amino acid sequence KASQDINSYLS (SEQ ID NO: 36), a CDR-L2 having the amino acid sequence RANRLVD (SEQ ID NO: 37), and a CDR-L3 having the amino acid sequence LQYGEFPPT (SEQ ID NO: 38);
wherein in each of said CDR-H1, said CDR-H2, said CDR-H3, said CDR-L1, said CDR-L2, and said CDR-L3, a single amino acid residue is optionally replaced by a different amino acid residue; or
(9) a heavy chain variable domain (VH) comprising a CDR-H1 having the amino acid sequence AYAMN (SEQ ID NO: 20), a CDR-H2 having the amino acid sequence RIRSKSNNYATYYGDSVKD (SEQ ID NO: 39), and a CDR-H3 having the amino acid sequence GREAYYRYDGDYYAMDY (SEQ ID NO: 8); and
a light chain variable domain (VL) comprising a CDR-L1 having the amino acid sequence KSSQSLLNSSNQKNYLA (SEQ ID NO: 40), a CDR-L2 having the amino acid sequence FASTRES (SEQ ID NO: 41), and a CDR-L3 having the amino acid sequence QQHYSTPYT (SEQ ID NO: 42); wherein in each of said CDR-H1, said CDR-H2, said CDR-H3, said CDR-L1, said CDR-L2, and said CDR-L3, a single amino acid residue is optionally replaced by a different amino acid residue; or
(10) a heavy chain variable domain (VH) comprising a CDR-H1 having the amino acid sequence AYAMN (SEQ ID NO: 20), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); wherein in each of said CDR-H1, said CDR-H2, said CDR-H3, said CDR-L1, said CDR-L2, and said CDR-L3, a single amino acid residue is optionally replaced by a different amino acid residue; or
(11) a heavy chain variable domain (VH) comprising a CDR-H1 having the amino acid sequence EYTIH (SEQ ID NO: 14), a CDR-H2 having the amino acid sequence GINPNNGNTRYDQKFKG (SEQ ID NO: 43), and a CDR-H3 having the amino acid sequence VARSSGSGPYAMDY (SEQ ID NO: 44); and
a light chain variable domain (VL) comprising a CDR-L1 having the amino acid sequence RCTQSLLHTNGDTYLH (SEQ ID NO: 45), a CDR-L2 having the amino acid sequence KVSNRFS (SEQ ID NO: 18), and a CDR-L3 having the amino acid sequence SQSTHVPYT (SEQ ID NO: 19); wherein in each of said CDR-H1, said CDR-H2, said CDR-H3, said CDR-L1, said CDR-L2, and said CDR-L3, a single amino acid residue is optionally replaced by a different amino acid residue; or
(12) a heavy chain variable domain (VH) comprising a CDR-H1 having the amino acid sequence GYNMH (SEQ ID NO: 46), a CDR-H2 having the amino acid sequence AISPGKGDTSYNLKFKG (SEQ ID NO: 47), and a CDR-H3 having the amino acid sequence SGGTPFAY (SEQ ID NO: 48); and
a light chain variable domain (VL) comprising a CDR-L1 having the amino acid sequence RSSQSLVHSNGDTYLY (SEQ ID NO: 49), a CDR-L2 having the amino acid sequence KVSNRFS (SEQ ID NO: 18), and a CDR-L3 having the amino acid sequence SQSTHVPYT (SEQ ID NO: 19); wherein in each of said CDR-H1, said CDR-H2, said CDR-H3, said CDR-L1, said CDR-L2, and said CDR-L3, a single amino acid residue is optionally replaced by a different amino acid residue; or
(13) a heavy chain variable domain (VH) comprising a CDR-H1 having the amino acid sequence ANAMN (SEQ ID NO: 50), a CDR-H2 having the amino acid sequence RIRSKSNNYATYYADSVKD (SEQ ID NO: 7), and a CDR-H3 having the amino acid sequence GSDNYIFYAMDY (SEQ ID NO: 51); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); wherein in each of said CDR-H1, said CDR-H2, said CDR-H3, said CDR-L1, said CDR-L2, and said CDR-L3, a single amino acid residue is optionally replaced by a different amino acid residue; or
(14) a heavy chain variable domain (VH) comprising a CDR-H1 having the amino acid sequence TNAMN (SEQ ID NO: 52), a CDR-H2 having the amino acid sequence RIRSKSNYYATYYADSVKD (SEQ ID NO: 53), and a CDR-H3 having the amino acid sequence GREMGNYYSMDY (SEQ ID NO: 54); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); wherein in each of said CDR-H1, said CDR-H2, said CDR-H3, said CDR-L1, said CDR-L2, and said CDR-L3, a single amino acid residue is optionally replaced by a different amino acid residue; or
(15) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PTYPGSSGFAY (SEQ ID NO: 55); and
A light chain variable domain (VL) comprising a CDR-L1 having the amino acid sequence RASQDISNYLN (SEQ ID NO: 56), a CDR-L2 having the amino acid sequence YTSRLHS (SEQ ID NO: 57), and a CDR-L3 having the amino acid sequence QQGNKFPPT (SEQ ID NO: 58); wherein in each of said CDR-H1, said CDR-H2, said CDR-H3, said CDR-L1, said CDR-L2, and said CDR-L3, a single amino acid residue is optionally replaced by a different amino acid residue; or
(16) a heavy chain variable domain (VH) comprising a CDR-H1 having the amino acid sequence EYTMH (SEQ ID NO: 59), a CDR-H2 having the amino acid sequence GINPNNGDTNYNQKFMG (SEQ ID NO: 60), and a CDR-H3 having the amino acid sequence RLLRRGAMDY (SEQ ID NO: 61); and
a light chain variable domain (VL) comprising a CDR-L1 having the amino acid sequence RASENVEYYGTSLMQ (SEQ ID NO: 62), a CDR-L2 having the amino acid sequence AASNVDS (SEQ ID NO: 63), and a CDR-L3 having the amino acid sequence QQSRKVPWT (SEQ ID NO: 64); wherein in each of said CDR-H1, said CDR-H2, said CDR-H3, said CDR-L1, said CDR-L2, and said CDR-L3, a single amino acid residue is optionally replaced by a different amino acid residue; or
(17) a heavy chain variable domain (VH) comprising a CDR-H1 having the amino acid sequence GSYMH (SEQ ID NO: 65), a CDR-H2 having the amino acid sequence RINPYNGATSYNQNFKD (SEQ ID NO: 66), and a CDR-H3 having the amino acid sequence TLLRLLDY (SEQ ID NO: 67); and
a light chain variable domain (VL) comprising a CDR-L1 having the amino acid sequence RSSKSLLHSNGITYLY (SEQ ID NO: 68), a CDR-L2 having the amino acid sequence QMSNLAS (SEQ ID NO: 69), and a CDR-L3 having the amino acid sequence AQNLELPWT (SEQ ID NO: 70); wherein in each of said CDR-H1, said CDR-H2, said CDR-H3, said CDR-L1, said CDR-L2, and said CDR-L3, a single amino acid residue is optionally replaced by a different amino acid residue; or
(18) a heavy chain variable domain (VH) comprising a CDR-H1 having the amino acid sequence SYWMH (SEQ ID NO: 71), a CDR-H2 having the amino acid sequence NIWPGSASTNYDEKFKN (SEQ ID NO: 72), and a CDR-H3 having the amino acid sequence GGKGAMDY (SEQ ID NO: 73); and
a light chain variable domain (VL) comprising a CDR-L1 having the amino acid sequence RSSQSLENSYGNTYLN (SEQ ID NO: 74), a CDR-L2 having the amino acid sequence RVSNRFS (SEQ ID NO: 75), and a CDR-L3 having the amino acid sequence LQVTHVPPT (SEQ ID NO: 76); wherein in each of said CDR-H1, said CDR-H2, said CDR-H3, said CDR-L1, said CDR-L2, and said CDR-L3, a single amino acid residue is optionally replaced by a different amino acid residue; or
(19) a heavy chain variable domain (VH) comprising a CDR-H1 having the amino acid sequence TYAMH (SEQ ID NO: 77), a CDR-H2 having the amino acid sequence HINPSSGYSNYNQKFKD (SEQ ID NO: 78), and a CDR-H3 having the amino acid sequence SEVRRGYFDV (SEQ ID NO: 79); and
a light chain variable domain (VL) comprising a CDR-L1 having the amino acid sequence RTSQDIRNYLN (SEQ ID NO: 80), a CDR-L2 having the amino acid sequence YTSRLHS (SEQ ID NO: 57), and a CDR-L3 having the amino acid sequence QQGNTLPPT (SEQ ID NO: 81); wherein in each of said CDR-H1, said CDR-H2, said CDR-H3, said CDR-L1, said CDR-L2, and said CDR-L3, a single amino acid residue is optionally replaced by a different amino acid residue; or
(20) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDV (SEQ ID NO: 124); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11); wherein in each of said CDR-H1, said CDR-H2, said CDR-H3, said CDR-L1, said CDR-L2, and said CDR-L3, a single amino acid residue is optionally replaced by a different amino acid residue; or
(21) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
A light chain variable domain (VL) comprising a CDR-L1 having the amino acid sequence KSLLHSNGNTY (SEQ ID NO: 125), a CDR-L2 having the amino acid sequence RVS, and a CDR-L3 having the amino acid sequence MQHREYPFT (SEQ ID NO: 23);
wherein in each of said CDR-H1, said CDR-H2, said CDR-H3, said CDR-L1, said CDR-L2, and said CDR-L3, a single amino acid residue is optionally replaced by a different amino acid residue; or
(22) a heavy chain variable domain (VH) comprising a CDR-H1 having the amino acid sequence GYTFTEYT (SEQ ID NO: 128), a CDR-H2 having the amino acid sequence INPNNGNT (SEQ ID NO: 129), and a CDR-H3 having the amino acid sequence ARVARSSGSGPYAMDY (SEQ ID NO: 130); and
a light chain variable domain (VL) comprising a CDR-L1 having the amino acid sequence QSLLHTNGDTY (SEQ ID NO: 131), a CDR-L2 having the amino acid sequence KVS, and a CDR-L3 having the amino acid sequence SQSTHVPYT (SEQ ID NO: 19);
wherein in each of said CDR-H1, said CDR-H2, said CDR-H3, said CDR-L1, said CDR-L2, and said CDR-L3, a single amino acid residue is optionally replaced by a different amino acid residue; or
(23) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDY (SEQ ID NO: 133); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23);
wherein in each of said CDR-H1, said CDR-H2, said CDR-H3, said CDR-L1, said CDR-L2, and said CDR-L3, a single amino acid residue is optionally replaced by a different amino acid residue; or
(24) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDY (SEQ ID NO: 133); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11);
wherein in each of said CDR-H1, said CDR-H2, said CDR-H3, said CDR-L1, said CDR-L2, and said CDR-L3, a single amino acid residue is optionally replaced by a different amino acid residue; or
(25) a heavy chain variable domain (VH) comprising a CDR-H1 having the amino acid sequence GFTFSNYR (SEQ ID NO: 134), a CDR-H2 having the amino acid sequence IKVKSDNYGA (SEQ ID NO: 135), and a CDR-H3 having the amino acid sequence SSPTYPGSSGFAY (SEQ ID NO: 136); and
a light chain variable domain (VL) comprising a CDR-L1 having the amino acid sequence QDISNY (SEQ ID NO: 137), a CDR-L2 having the amino acid sequence YTS, and a CDR-L3 having the amino acid sequence QQGNKFPPT (SEQ ID NO: 58);
wherein in each of said CDR-H1, said CDR-H2, said CDR-H3, said CDR-L1, said CDR-L2, and said CDR-L3, a single amino acid residue is optionally replaced by a different amino acid residue.

5. The antibody or antigen-binding fragment according to any one of claims 1 to 4, wherein the antibody or antigen-binding fragment comprises:
(1) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23); or
(2) a heavy chain variable domain (VH) comprising a CDR-H1 having the amino acid sequence PYAMN (SEQ ID NO: 6), a CDR-H2 having the amino acid sequence RIRSKSNNYATYYADSVKD (SEQ ID NO: 7), and a CDR-H3 having the amino acid sequence GREAYYRYDGDYYAMDY (SEQ ID NO: 8); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(3) a heavy chain variable domain (VH) comprising a CDR-H1 having the amino acid sequence TYAMN (SEQ ID NO: 12), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(4) a heavy chain variable domain (VH) comprising a CDR-H1 having the amino acid sequence EYTIH (SEQ ID NO: 14), a CDR-H2 having the amino acid sequence GINPNGDTRYDQKFKG (SEQ ID NO: 15), and a CDR-H3 having the amino acid sequence VARFYGISPYAMDY (SEQ ID NO: 16); and
a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQSTHVPYT (SEQ ID NO: 19); or
(5) a heavy chain variable domain (VH) comprising a CDR-H1 having the amino acid sequence AYAMN (SEQ ID NO: 20), a CDR-H2 having the amino acid sequence RIRSKSNDYATYYGDSVKD (SEQ ID NO: 21), and a CDR-H3 having the amino acid sequence GREAYYRYDGGYYAMDY (SEQ ID NO: 13); and
a light chain variable domain (VL) comprising a CDR-L1 having the amino acid sequence TSSKSLLHSNGNTYLY (SEQ ID NO: 22), a CDR-L2 having the amino acid sequence RMSNLAS (SEQ ID NO: 10), and a CDR-L3 having the amino acid sequence MQHREYPFT (SEQ ID NO: 23); or
(6) a heavy chain variable domain (VH) comprising a CDR-H1 having the amino acid sequence DYVVT (SEQ ID NO: 24), a CDR-H2 having the amino acid sequence VIWGGGNTYYNSDLKS (SEQ ID NO: 25), and a CDR-H3 having the amino acid sequence RHRDYALDY (SEQ ID NO: 26); and
a light chain variable domain (VL) comprising a CDR-L1 having the amino acid sequence RSSQSLVYSNGNTYLH (SEQ ID NO: 27), a CDR-L2 having the amino acid sequence KVSNRFS (SEQ ID NO: 18), and a CDR-L3 having the amino acid sequence SQSTYVPPT (SEQ ID NO: 28); or
(7) a heavy chain variable domain (VH) comprising a CDR-H1 having the amino acid sequence DYTLH (SEQ ID NO: 29), a CDR-H2 having the amino acid sequence GITPKNGDTRYDPRFKD (SEQ ID NO: 30), and a CDR-H3 having the amino acid sequence VARFYGVSPYAMDY (SEQ ID NO: 31); and
a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQTTHVPYT (SEQ ID NO: 32); or
(8) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PDGFSPFVY (SEQ ID NO: 35); and
a light chain variable domain (VL) comprising a CDR-L1 having the amino acid sequence KASQDINSYLS (SEQ ID NO: 36), a CDR-L2 having the amino acid sequence RANRLVD (SEQ ID NO: 37), and a CDR-L3 having the amino acid sequence LQYGEFPPT (SEQ ID NO: 38); or
(9) a heavy chain variable domain (VH) comprising a CDR-H1 having the amino acid sequence AYAMN (SEQ ID NO: 20), a CDR-H2 having the amino acid sequence RIRSKSNNYATYYGDSVKD (SEQ ID NO: 39), and a CDR-H3 having the amino acid sequence GREAYYRYDGDYYAMDY (SEQ ID NO: 8); and
a light chain variable domain (VL) comprising a CDR-L1 having the amino acid sequence KSSQSLLNSSNQKNYLA (SEQ ID NO: 40), a CDR-L2 having the amino acid sequence FASTRES (SEQ ID NO: 41), and a CDR-L3 having the amino acid sequence QQHYSTPYT (SEQ ID NO: 42); or
(10) a heavy chain variable domain (VH) comprising a CDR-H1 having the amino acid sequence AYAMN (SEQ ID NO: 20), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(11) a heavy chain variable domain (VH) comprising a CDR-H1 having the amino acid sequence EYTIH (SEQ ID NO: 14), a CDR-H2 having the amino acid sequence GINPNNGNTRYDQKFKG (SEQ ID NO: 43), and a CDR-H3 having the amino acid sequence VARSSGSGPYAMDY (SEQ ID NO: 44); and
a light chain variable domain (VL) comprising a CDR-L1 having the amino acid sequence RCTQSLLHTNGDTYLH (SEQ ID NO: 45), a CDR-L2 having the amino acid sequence KVSNRFS (SEQ ID NO: 18), and a CDR-L3 having the amino acid sequence SQSTHVPYT (SEQ ID NO: 19); or
(12) a heavy chain variable domain (VH) comprising a CDR-H1 having the amino acid sequence GYNMH (SEQ ID NO: 46), a CDR-H2 having the amino acid sequence AISPGKGDTSYNLKFKG (SEQ ID NO: 47), and a CDR-H3 having the amino acid sequence SGGTPFAY (SEQ ID NO: 48); and
a light chain variable domain (VL) comprising a CDR-L1 having the amino acid sequence RSSQSLVHSNGDTYLY (SEQ ID NO: 49), a CDR-L2 having the amino acid sequence KVSNRFS (SEQ ID NO: 18), and a CDR-L3 having the amino acid sequence SQSTHVPYT (SEQ ID NO: 19); or
(13) a heavy chain variable domain (VH) comprising a CDR-H1 having the amino acid sequence ANAMN (SEQ ID NO: 50), a CDR-H2 having the amino acid sequence RIRSKSNNYATYYADSVKD (SEQ ID NO: 7), and a CDR-H3 having the amino acid sequence GSDNYIFYAMDY (SEQ ID NO: 51); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(14) a heavy chain variable domain (VH) comprising a CDR-H1 having the amino acid sequence TNAMN (SEQ ID NO: 52), a CDR-H2 having the amino acid sequence RIRSKSNYYATYYADSVKD (SEQ ID NO: 53), and a CDR-H3 having the amino acid sequence GREMGNYYSMDY (SEQ ID NO: 54); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(15) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PTYPGSSGFAY (SEQ ID NO: 55); and
a light chain variable domain (VL) comprising a CDR-L1 having the amino acid sequence RASQDISNYLN (SEQ ID NO: 56), a CDR-L2 having the amino acid sequence YTSRLHS (SEQ ID NO: 57), and a CDR-L3 having the amino acid sequence QQGNKFPPT (SEQ ID NO: 58); or
(16) a heavy chain variable domain (VH) comprising a CDR-H1 having the amino acid sequence EYTMH (SEQ ID NO: 59), a CDR-H2 having the amino acid sequence GINPNNGDTNYNQKFMG (SEQ ID NO: 60), and a CDR-H3 having the amino acid sequence RLLRRGAMDY (SEQ ID NO: 61); and
a light chain variable domain (VL) comprising a CDR-L1 having the amino acid sequence RASENVEYYGTSLMQ (SEQ ID NO: 62), a CDR-L2 having the amino acid sequence AASNVDS (SEQ ID NO: 63), and a CDR-L3 having the amino acid sequence QQSRKVPWT (SEQ ID NO: 64); or
(17) a heavy chain variable domain (VH) comprising a CDR-H1 having the amino acid sequence GSYMH (SEQ ID NO: 65), a CDR-H2 having the amino acid sequence RINPYNGATSYNQNFKD (SEQ ID NO: 66), and a CDR-H3 having the amino acid sequence TLLRLLDY (SEQ ID NO: 67); and
a light chain variable domain (VL) comprising a CDR-L1 having the amino acid sequence RSSKSLLHSNGITYLY (SEQ ID NO: 68), a CDR-L2 having the amino acid sequence QMSNLAS (SEQ ID NO: 69), and a CDR-L3 having the amino acid sequence AQNLELPWT (SEQ ID NO: 70); or
(18) a heavy chain variable domain (VH) comprising a CDR-H1 having the amino acid sequence SYWMH (SEQ ID NO: 71), a CDR-H2 having the amino acid sequence NIWPGSASTNYDEKFKN (SEQ ID NO: 72), and a CDR-H3 having the amino acid sequence GGKGAMDY (SEQ ID NO: 73); and
a light chain variable domain (VL) comprising a CDR-L1 having the amino acid sequence RSSQSLENSYGNTYLN (SEQ ID NO: 74), a CDR-L2 having the amino acid sequence RVSNRFS (SEQ ID NO: 75), and a CDR-L3 having the amino acid sequence LQVTHVPPT (SEQ ID NO: 76); or
(19) a heavy chain variable domain (VH) comprising a CDR-H1 having the amino acid sequence TYAMH (SEQ ID NO: 77), a CDR-H2 having the amino acid sequence HINPSSGYSNYNQKFKD (SEQ ID NO: 78), and a CDR-H3 having the amino acid sequence SEVRRGYFDV (SEQ ID NO: 79); and
a light chain variable domain (VL) comprising a CDR-L1 having the amino acid sequence RTSQDIRNYLN (SEQ ID NO: 80), a CDR-L2 having the amino acid sequence YTSRLHS (SEQ ID NO: 57), and a CDR-L3 having the amino acid sequence QQGNTLPPT (SEQ ID NO: 81); or
(20) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDV (SEQ ID NO: 124); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11); or
(21) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
a light chain variable domain (VL) comprising a CDR-L1 having the amino acid sequence KSLLHSNGNTY (SEQ ID NO: 125), a CDR-L2 having the amino acid sequence RVS, and a CDR-L3 having the amino acid sequence MQHREYPFT (SEQ ID NO: 23); or
(22) a heavy chain variable domain (VH) comprising a CDR-H1 having the amino acid sequence GYTFTEYT (SEQ ID NO: 128), a CDR-H2 having the amino acid sequence INPNNGNT (SEQ ID NO: 129), and a CDR-H3 having the amino acid sequence ARVARSSGSGPYAMDY (SEQ ID NO: 130); and
a light chain variable domain (VL) comprising a CDR-L1 having the amino acid sequence QSLLHTNGDTY (SEQ ID NO: 131), a CDR-L2 having the amino acid sequence KVS, and a CDR-L3 having the amino acid sequence SQSTHVPYT (SEQ ID NO: 19); or
(23) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDY (SEQ ID NO: 133); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23); or
(24) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDY (SEQ ID NO: 133); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11); or
(25) a heavy chain variable domain (VH) comprising a CDR-H1 having the amino acid sequence GFTFSNYR (SEQ ID NO: 134), a CDR-H2 having the amino acid sequence IKVKSDNYGA (SEQ ID NO: 135), and a CDR-H3 having the amino acid sequence SSPTYPGSSGFAY (SEQ ID NO: 136); and
a light chain variable domain (VL) comprising a CDR-L1 having the amino acid sequence QDISNY (SEQ ID NO: 137), a CDR-L2 having the amino acid sequence YTS, and a CDR-L3 having the amino acid sequence QQGNKFPPT (SEQ ID NO: 58).

6. The antibody or antigen-binding fragment according to any one of claims 1 to 5, wherein the antibody or antigen-binding fragment comprises:
(1) a heavy chain variable domain (VH) having an amino acid sequence with at least 80% sequence identity to the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SS (SEQ ID NO: 140); and
a light chain variable domain (VL) having an amino acid sequence with at least 80% sequence identity the to amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIK (SEQ ID NO: 141); or
(2) a heavy chain variable domain (VH) having an amino acid sequence with at least 80% sequence identity acid to the amino sequence MLLGLKWVFFVVFYQGVLCEVQLLESGGGLVQPKGSLKLSCAASGFTFNPYAMNWVRQAPGRG LEWVARIRSKSNNYATYYADSVKDRFTISRDDSQDMLYLQMNNLKTEDTAMYYCVRGREAYYRY DGDYYAMDYWGQGTSVTVSS (SEQ ID NO: 82); and
a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
(3) a heavy chain variable domain (VH) having an amino acid sequence with at least 80% sequence identity to the amino acid sequence MLLGLKWVFFVVFYQGVHCEVQLVESGGGLVQPKGSLKLSCAASGFTFNTYAMNWVRQAPGK GLEWVARIRSKSNNYATYYADSVKDRFTISRDDSQSMLYLQMNNLKTEDTAMYYCVRGREAYYR YDGGYYAMDYWGQGTSVTVSS (SEQ ID NO: 84); and
a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
(4) a heavy chain variable domain (VH) having an amino acid sequence with at least 80% sequence identity to the amino acid sequence MGWSWIFLFLLSGTAGVLSKVQLQQSGPELVKPGASVKISCKTSGYTFTEYTIHWVQQIHGKSPE WIGGINPNGDTRYDQKFKGKATLTIDKSSSTAYMELRSLTSEDSAVYYCARVARFYGISPYAMDY WGQGTSVTVSS (SEQ ID NO: 85); and
a light chain variable domain (VL) having an amino acid sequence with at least 80% sequence identity to the amino acid sequence MKLPVRLLVLMFWIPASSSDVVMTQTPLSLPVSLGDQASISCRCTQSLLHSNGDTYLHWYLQKP GQSPNLLIYKVSNRFSGVPDRFSGSGSGTDFTLKINRVEAEDLGVYFCSQSTHVPYTFGGGTNLE IK (SEQ ID NO: 86); or
(5) a heavy chain variable domain (VH) having an amino acid sequence with at least 80% sequence identity to the amino acid sequence MLLGLKWVFFVVFYQGVHCEVQLVESGGGLVQPTGSLRLSCAASGFTFNAYAMNWVRQAPGK GLEWVARIRSKSNDYATYYGDSVKDRFTISRDDSQSMLYLQMNNLKTEDTAMYYCVRGREAYY RYDGGYYAMDYWGQGTSVTVSS (SEQ ID NO: 87); and
a light chain variable domain (VL) having an amino acid sequence with at least 80% sequence identity to the amino acid sequence MRCLAEFLGLLVLWIPGAIGDIVMTQAAPSVSVTPGESVSISCTSSKSLLHSNGNTYLYWFLQRPG QSPQLLIYRMSNLASGVPDRFSGSGSGTAFTLRISRVEAEDVGVYYCMQHREYPFTFGSGTKLEI K (SEQ ID NO: 88); or
(6) a heavy chain variable domain (VH) having an amino acid sequence with at least 80% sequence identity to the amino acid sequence MAVLGLLLCLVTFPSCVLSQVQLKESGPGLVAPSQSLSITCTVSGLSMNDYVVTWIRQPPGKGLE WLGVIWGGGNTYYNSDLKSRLSITKDNSKSQVFFKMSSLQTDDTAVYYCARRHRDYALDYWGQ GISVTVSS (SEQ ID NO: 89); and
a light chain variable domain (VL) having an amino acid sequence with at least 80% sequence identity to the amino acid sequence MKLPVRLLVLMFWIPASSSDVVMTQTPLSLPVSLGDQASISCRSSQSLVYSNGNTYLHWYLQKP GQSPKLLIYKVSNRFSGVPDRFSGSGSGTDFTLKISRVEAEDLGVYFCSQSTYVPPTFGGGTKLEI K (SEQ ID NO: 90); or
(7) a heavy chain variable domain (VH) having an amino acid sequence with at least 80% sequence identity to the amino acid sequence MGWSWIFLFLLSGTAGVLSEVQLQQSGPELLKPGTSVKISCTTSGYTFSDYTLHWVKQSHGKSL EWIGGITPKNGDTRYDPRFKDKATLTIDKSSSAAYMELRSLTSEDSAVYYCARVARFYGVSPYAM DYWGQGASVTVSS (SEQ ID NO: 91); and
a light chain variable domain (VL) having an amino acid sequence with at least 80% sequence identity to the amino acid sequence MKLPVRLLVLMFWIPVSNSDVVMTQTPLSLPVSLGDQASISCRCTQSLLHSNGDTYLHWYLQKP GQSPKLLIYKVSNRFSGVPDRFSGSGSGTDFTLEITRVEAEDLGVYFCSQTTHVPYTFGGGTKLEI K (SEQ ID NO: 92); or
(8) a heavy chain variable domain (VH) having an amino acid sequence with at least 80% sequence identity to the amino acid sequence MELGLSWVFLVALLNGVQCQVHLVETGGGLVRPGNSLKLSCVTSGFTLSNYRMHWLRQPPGKR LEWIAVIKVKSDNYGANYAESVKGRFTISRDDAKSSVYLQMNRLREEDTATYYCSRPDGFSPFVY WGQGTLVTVSA (SEQ ID NO: 93); and
a light chain variable domain (VL) having an amino acid sequence with at least 80% sequence identity to the amino acid sequence MDMRTPAQFLGILLLWFPGIKCDIKMTQSPSSMYASLGERVTITCKASQDINSYLSWFQQKPGKS PKTLIYRANRLVDGVPSRFSGSGSGQDYFLTISSLEYEDMGIYYCLQYGEFPPTFGAGTKLELK (SEQ ID NO: 94); or
(9) a heavy chain variable domain (VH) having an amino acid sequence with at least 80% sequence identity to the amino acid sequence MLLGLKWVFFVVFYQGVHCEVQLVESGGGLVQPKGSLRLSCAASGFTFNAYAMNWVRQAPGK GLEWVARIRSKSNNYATYYGDSVKDRFTISRDDSQTMLYLQMNNLKTEDTAMYYCVRGREAYYR YDGDYYAMDYWGQGTSVTVSS (SEQ ID NO: 95); and
a light chain variable domain (VL) having an amino acid sequence with at least 80% sequence identity to the amino acid sequence MESQTQVLMFLLLWVSGACADIVMTQSPSSLAMSVGQKVTMSCKSSQSLLNSSNQKNYLAWYQ QKPGQSPKLLVYFASTRESGVPDRFIGSGSGTDFTLTISSVQAEDLADYFCQQHYSTPYTFGGGT KLEIK (SEQ ID NO: 96); or
(10) a heavy chain variable domain (VH) having an amino acid sequence with at least 80% sequence identity to the amino acid sequence MLLGLKWVFFVVFYQGVHCEVQLVESGGGLVQPKGSLKLSCAASGFTFNAYAMNWVRQAPGK GLEWLARIRSKSNNYATYYADSVKDRFTISRDDSQSMFYLQMNNLKSEDTAMYYCVRGREAYYR YDGGYYAMDYWGQGTSVTVSS (SEQ ID NO: 97); and
a light chain variable domain (VL) having an amino acid sequence with at least 80% sequence identity to the amino acid sequence MRCLAEFLGLLVLWIPGAIGEIVMTQAALSAPVTPGESVSMSCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 98); or
(11) a heavy chain variable domain (VH) having an amino acid sequence with at least 80% sequence identity to the amino acid sequence MGWSWIFLFLLSGTAGVLSEVQLQQSGPELVKPGASVKISCKTSGYTFTEYTIHWVKQSHGESLE WVGGINPNNGNTRYDQKFKGKATLTIDKSSSPAYMELRSLTSEDSAVYYCARVARSSGSGPYAM DYWGQGTSVTVSS (SEQ ID NO: 99); and
a light chain variable domain (VL) having an amino acid sequence with at least 80% sequence identity to the amino acid sequence MKLPVRLLVLMFWIPVSSSDVVMTQTPLSLPVSLGDQASISCRCTQSLLHTNGDTYLHWYLQKPG QSPKLLIYKVSNRFSGVPDRFSGSGSGTDFTLKISRVEAEDLGVYFCSQSTHVPYTFGGGTKLEIK (SEQ ID NO: 100); or
(12) a heavy chain variable domain (VH) having an amino acid sequence with at least 80% sequence identity to the amino acid sequence MGWSCIILFLVATATDVHSQVQLQQPGAELVKPGASVKMSCKATGYTFTGYNMHWVKQTPGQG LEWIGAISPGKGDTSYNLKFKGKATLTTDKSSSTAYMQLSSLTSADSAVYYCARSGGTPFAYWG QGTLVTVSA (SEQ ID NO: 101); and
a light chain variable domain (VL) having an amino acid sequence with at least 80% sequence identity to the amino acid sequence MKLPVRLLVLMFWIPASSSDVVMTQTPLSLPVSLGDQASISCRSSQSLVHSNGDTYLYWYLQKP GQSPKLLIYKVSNRFSGVPDRFSGSGSGTDFTLKINRVEAEDLGVYFCSQSTHVPYTFGGGTKLE IK (SEQ ID NO: 102); or
(13) a heavy chain variable domain (VH) having an amino acid sequence with at least 80% sequence identity to the amino acid sequence MLLGLKWVFFVVFYQGVHCEVQLVETGGGLVQPKGSLKLSCAASGFTFNANAMNWVRQAPGK GLEWVARIRSKSNNYATYYADSVKDRFTISRDDSQSMLYLQMNNLKTEDTAMYYCVRGSDNYIF YAMDYWGQGTSVTVSS (SEQ ID NO: 103); and
a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGGGTKL EIK (SEQ ID NO: 104); or
(14) a heavy chain variable domain (VH) having an amino acid sequence with at least 80% sequence identity to the amino acid sequence MLLGLKWVFFVVFYQGVHCEVQLVETGGGLVQPKGSLKLSCAASGFTFNTNAMNWVRQAPGK GLEWVARIRSKSNYYATYYADSVKDRFTISRDDSQSMLYLQMNNLKTEDTAMYYCVRGREMGN YYSMDYWGQGTSVTVSS (SEQ ID NO: 105); and
a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
(15) a heavy chain variable domain (VH) having an amino acid sequence with at least 80% sequence identity to the amino acid sequence MELGLSWVFLVALLNGVQCQVQLVETGGGLVRPGNSLKLSCVTSGFTFSNYRMHWLRQPPGKR LEWIAVIKVKSDNYGANYAESVKGRFTISRDDSKSSVYLQVNRLREEDTATYYCSSPTYPGSSGF AYWGQGTLVTVSA (SEQ ID NO: 106); and
a light chain variable domain (VL) having an amino acid sequence with at least 80% sequence identity to the amino acid sequence MMSSAQFLGLLLLCFQGTRCDIQMTQTTSSLSASLGDRVTISCRASQDISNYLNWYQQKPDGTV KLLIYYTSRLHSGVPSRFSGSGSGTDYSLTISNLEQEDIATYFCQQGNKFPPTFGGGTKLEIN (SEQ ID NO: 107); or
(16) a heavy chain variable domain (VH) having an amino acid sequence with at least 80% sequence identity to the amino acid sequence MGWSWIFLFLLSGTAGVLSEVQLQQSGPELVKPGASVKISCKTSGYTFTEYTMHWVKQSHGKSL EWIGGINPNNGDTNYNQKFMGKATLTVDKSSSTAYMELRSLTSEDSAVYYCVRRLLRRGAMDY WGQGTSVTVSS (SEQ ID NO: 108); and
a light chain variable domain (VL) having an amino acid sequence with at least 80% sequence identity to the amino acid sequence MESDTLLLWVLLLWVPGSTGDIVLTQSPASLAVSLGQRATISCRASENVEYYGTSLMQWFQQKP GQPPKLLIYAASNVDSGVPARFSGSGSGTDFSLNIHPVEEDDIAMYFCQQSRKVPWTFGGGTKL DIK (SEQ ID NO: 109); or
(17) a heavy chain variable domain (VH) having an amino acid sequence with at least 80% sequence identity to the amino acid sequence MGWSWIFLFLLSGTAGVLSEVQLQQSGPELVKPGASVKISCKASVYSFTGSYMHWVKQSHVKSL EWIGRINPYNGATSYNQNFKDKASLTVDKSSSTAYMELHSLTSEDSAVYYCATTLLRLLDYWGQ GTTLTVSS (SEQ ID NO: 110); and
a light chain variable domain (VL) having an amino acid sequence with at least 80% sequence identity to the amino acid sequence MRFSAQLLGLLVLWIPGSTADIVMTQAAFSNPVTLGTSASISCRSSKSLLHSNGITYLYWYLQKPG QSPQLLIYQMSNLASGVPDRESSSGSGTDFTLRISRVEAEDVGVYYCAQNLELPWTFGGGTKLEI K (SEQ ID NO: 111); or
(18) a heavy chain variable domain (VH) having an amino acid sequence with at least 80% sequence identity to the amino acid sequence MGWSSIILFLVATASGVHSQVQLQQPGSELVRPGASVKLSCKASGYTFTSYWMHWVKQRPGQG LEWIGNIWPGSASTNYDEKFKNKATLTVDTSSSTAYMQLSSLTSEDSAVYYCIRGGKGAMDYWS QGTSVTVSS (SEQ ID NO: 112); and
a light chain variable domain (VL) having an amino acid sequence with at least 80% sequence identity to the amino acid sequence MKLPVRLLVLMFWIPVSSSDVVMTQIPLSLPVSLGDQASISCRSSQSLENSYGNTYLNWYLQKPG QSPQLLIYRVSNRFSGVLDRFSGSGSGTDFTLKISRVEAEDLGVYFCLQVTHVPPTFGAGTKLEL K (SEQ ID NO: 113); or
(19) a heavy chain variable domain (VH) having an amino acid sequence with at least 80% sequence identity to the amino acid sequence MERHWIFLLLLSVTAGVHSQVQVQQSGAELARPGASVKMSCKASGYTFTTYAMHWVKQRPGQ GLEWIGHINPSSGYSNYNQKFKDKATLTADKSSSTAYMQLSSLTSEDSAVYYCARSEVRRGYFD VWGAGTTVTVSS (SEQ ID NO: 114); and
a light chain variable domain (VL) having an amino acid sequence with at least 80% sequence identity to the amino acid sequence MMSSAQFLGLLLLCFQGTRCDIQMTQTTSSLSASLGDRVTISCRTSQDIRNYLNWYQQKPDGTV KLLISYTSRLHSGVPSRFSGSGSGTDYSLTISNLEQEDFATYFCQQGNTLPPTFGGGTRVEIK (SEQ ID NO: 115); or
(20) a heavy chain variable domain (VH) having an amino acid sequence with at least 80% sequence identity amino to the acid sequence EVOLVESGGGLVQPGRSLRLSCTASGFTFNTYAMNWVRQAPGKGLEWVARIRSKSNNYATYYA ASVKGRFTISRDDSKSIAYLQMNSLKTEDTAVYYCVRGREAYYRYDGGYYAMDVWGQGTTVTV SS (SEQ ID NO: 138); and
a light chain variable domain (VL) having an amino acid sequence with at least 80% sequence identity to the amino acid sequence DIVMTQAAPSLPVTPGESASISCRSSKSLLHSNGNTYLYWFLQKPGQSPQLLIYRMSNLASGVPD RFSGSGSGTAFTLKISRVEAEDVGVYYCMQHLEYPFTFGSGTKLEIK (SEQ ID NO: 139); or
(21) a heavy chain variable domain (VH) having an amino acid sequence with at least 80% sequence identity to the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SS (SEQ ID NO: 140); and
a light chain variable domain (VL) having an amino acid sequence with at least 80% sequence identity to the amino acid sequence DIVMTQSPLSLPVTPGEPASISCRSSKSLLHSNGNTYLYWFLQKPGQSPQLLIYRVSNLASGVPD RFSGSGSGTDFTLKISRVEAEDVGVYYCMQHREYPFTFGQGTKLEIK (SEQ ID NO: 142); or
(22) a heavy chain variable domain (VH) having an amino acid sequence with at least 80% sequence identity to the acid sequence amino QVQLVQSGPEVVKPGASVKVSCKTSGYTFTEYTIHWVRQAPGQSLEWVGGINPNNGNTRYDQK FQGRVTITRDKSASTAYMELRSLTSEDTAVYYCARVARSSGSGPYAMDYWGQGTTVTVSS (SEQ ID NO: 143); and
a light chain variable domain (VL) having an amino acid sequence with at least 80% sequence identity to the amino acid sequence DWVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIK (SEQ ID NO: 144); or
(23) a heavy chain variable domain (VH) having an amino acid sequence with at least 80% sequence identity to the amino acid sequence QVQLQQSGPEVVKPGASVKVSCKTSGYTFTEYTIHWVRQSHGQSLEWVGGINPNNGNTRYDQK FKGRVTITIDKSSSTAYMELRSLTSEDTAVYYCARVARSSGSGPYAMDYWGQGTTVTVSS (SEQ ID NO: 145); and
a light chain variable domain (VL) having an amino acid sequence with at least 80% sequence identity to the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIK (SEQ ID NO: 144); or
(24) a heavy chain variable domain (VH) having an amino acid sequence with at least 80% sequence identity to the amino acid sequence QVQLVESGGGVVQPGRSLRLSCAASGFTFSNYRMHWVRQAPGKGLEWIAVIKVKSDNYGANYA DSVKGRFTISRDNSKNTLYLQMNSLRAEDTAVYYCSSPTYPGSSGFAYWGQGTLVTVSS (SEQ ID NO: 146); and
a light chain variable domain (VL) having an amino acid sequence with at least 80% sequence identity to the amino acid sequence DIQMTQSPSSLSASVGDRVTITCRASQDISNYLNWYQQKPDGTVKLLIYYTSRLHTGVPSRFSGS GSGTDYTLTISNLQQEDIATYFCQQGNKFPPTFGGGTKVEIN (SEQ ID NO: 132); or
(25) a heavy chain variable domain (VH) having an amino acid sequence with at least 80% sequence identity to the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSS (SEQ ID NO: 147); and
a light chain variable domain (VL) having an amino acid sequence with at least 80% sequence identity to the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIK (SEQ ID NO: 148); or
(26) a heavy chain variable domain (VH) having an amino acid sequence with at least 80% sequence identity to the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSS (SEQ ID NO: 149); and
a light chain variable domain (VL) having an amino acid sequence with at least 80% sequence identity to the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIK (SEQ ID NO: 150); or
(27) a heavy chain variable domain (VH) having an amino acid sequence with at least 80% sequence identity to the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSS (SEQ ID NO: 151); and
a light chain variable domain (VL) having an amino acid sequence with at least 80% sequence identity to the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIK (SEQ ID NO: 152); or
(28) a heavy chain variable domain (VH) having an amino acid sequence with at least 80% sequence identity to the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSA (SEQ ID NO: 153); and
a light chain variable domain (VL) having an amino acid sequence with at least 80% sequence identity to the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEIN (SEQ ID NO: 154).

7. The antibody or antigen-binding fragment according to any one of claims 1 to 6, wherein the antibody or antigen-binding fragment comprises:
(1) a heavy chain (HC) having the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPDVFLFP PKPKDTLMISRTPEVTCVVVDVSHEDPEVKFNWYVDGVEVHNAKTKPREEQYNSTYRVVSVLTV LHQDWLNGKEYKCKVSNKALPAPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 163); and
a light chain (LC) having the amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 162); or
(2) a heavy chain (HC) having the amino acid sequence EVOLVESGGGLVQPGRSLRLSCTASGFTFNTYAMNWVRQAPGKGLEWVARIRSKSNNYATYYA ASVKGRFTISRDDSKSIAYLQMNSLKTEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFLFP PKPKDTLMISRTPEVTCWWVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 159); and
a light chain (LC) having the amino acid sequence DIVMTQAAPSLPVTPGESASISCRSSKSLLHSNGNTYLYWFLQKPGQSPQLLIYRMSNLASGVPD RFSGSGSGTAFTLKISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 160); or
(3) a heavy chain (HC) having the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 161); and
a light chain (LC) having the amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIKRTVAAPSVFIFPPSDEQLK SGTASWVCLLNNFYPREAKVQWKVDNALQSGNSQESVTEQDSKDSTYSLSSTLTLSKADYEKHK VYACEVTHQGLSSPVTKSFNRGEC (SEQ ID NO: 162); or
(4) a heavy chain (HC) having the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 161); and
a light chain (LC) having the amino acid sequence DIVMTQSPLSLPVTPGEPASISCRSSKSLLHSNGNTYLYWFLQKPGQSPQLLIYRVSNLASGVPD RFSGSGSGTDFTLKISRVEAEDVGVYYCMQHREYPFTFGQGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 164); or
(5) a heavy chain (HC) having the amino acid sequence QVQLVQSGPEVVKPGASVKVSCKTSGYTFTEYTIHWVRQAPGQSLEWVGGINPNNGNTRYDQK FQGRVTITRDKSASTAYMELRSLTSEDTAVYYCARVARSSGSGPYAMDYWGQGTTVTVSSASTK GPSVFPLAPSSKSTSGGTAALGCLVKDYFPEPVTVSWNSGALTSGVHTFPAVLQSSGLYSLSSV VTVPSSSLGTQTYICNVNHKPSNTKVDKKVEPKSCDKTHTCPPCPAPELLGGPSVFLFPPKPKDT LMISRTPEVTCVVVDVSHEDPEVKFNWYVDGVEVHNAKTKPREEQYNSTYRVVSVLTVLHQDWL NGKEYKCKVSNKALPAPIEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 165); and
a light chain (LC) having the amino acid sequence DWVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIKRTVAAPSVFIFPPSDEQ LKSGTASWVCLLNNFYPREAKVQWKVDNALQSGNSQESVTEQDSKDSTYSLSSTLTLSKADYEK HKVYACEVTHQGLSSPVTKSFNRGEC (SEQ ID NO: 166); or
(6) a heavy chain (HC) having the amino acid sequence QVQLQQSGPEVVKPGASVKVSCKTSGYTFTEYTIHWVRQSHGQSLEWVGGINPNNGNTRYDQK FKGRVTITIDKSSSTAYMELRSLTSEDTAVYYCARVARSSGSGPYAMDYWGQGTTVTVSSASTK GPSVFPLAPSSKSTSGGTAALGCLVKDYFPEPVTVSWNSGALTSGVHTFPAVLQSSGLYSLSSV VTVPSSSLGTQTYICNVNHKPSNTKVDKKVEPKSCDKTHTCPPCPAPELLGGPSVFLFPPKPKDT LMISRTPEVTCVVVDVSHEDPEVKFNWYVDGVEVHNAKTKPREEQYNSTYRVVSVLTVLHQDWL NGKEYKCKVSNKALPAPIEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 167); and
a light chain (LC) having the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO: 166); or
(7) a heavy chain (HC) having the amino acid sequence QVQLVESGGGVVQPGRSLRLSCAASGFTFSNYRMHWVRQAPGKGLEWIAVIKVKSDNYGANYA DSVKGRFTISRDNSKNTLYLQMNSLRAEDTAVYYCSSPTYPGSSGFAYWGQGTLVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPSVFLFPPKPKDTLM ISRTPEVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 168); and
a light chain (LC) having the amino acid sequence DIQMTQSPSSLSASVGDRVTITCRASQDISNYLNWYQQKPDGTVKLLIYYTSRLHTGVPSRFSGS GSGTDYTLTISNLQQEDIATYFCQQGNKFPPTFGGGTKVEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 169); or
(8) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPSVFLF PPKPKDTLMISRTPEVTCVVVDVSHEDPEVKFNWYVDGVEVHNAKTKPREEQYNSTYRVVSVLT VLHQDWLNGKEYKCKVSNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 170); and
a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(9) a heavy chain (HC) having the amino acid sequence EVQLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPAPEEKTISKAKGQPREPQVYTLPPSRDELTKNQVSLTCLVKG FYPSDIAVEWESNGQPENNYKTTPPVLDSDGSFFLYSKLTVDKSRWQQGNVFSCSVMHEALHN HYTQKSLSLSPG (SEQ ID NO: 172); and
a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(10) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPLPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 173); and
a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(11) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSWVTVPSSSLGTQTYICNVNHKPSNTKVDKKVEPKSCDKTHTCPPCPAPELVGGPSVFLL PPKPKDTLMISRTPEVTCWWVDVSHEDPEVKFNWYVDGVEVHNAKTKPPEEQYNSTLRVVSVLT VLHQDWLNGKEYKCKVSNKALPAPIEKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 174); and
a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASWVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(12) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNWALPAPISKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 175); and
a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(13) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPSVFLFPPKPKDTLM ISRTPEVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 176); and
a light chain (LC) having the amino acid sequence DWVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASWVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(14) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPDVFLFPPKPKDTLM ISRTPEVTCVVVDVSHEDPEVKFNWYVDGVEVHNAKTKPREEQYNSTYRVVSVLTVLHQDWLN GKEYKCKVSNKALPAPEEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 178); and
a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(15) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPDVFLFPPKPKDTLM ISRTPEVTCVVVDVSHEDPEVKFNWYVDGVEVHNAKTKPREEQYNSTYRVVSVLTVLHQDWLN GKEYKCKVSNKALPLPEEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 179); and
a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(16) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELVGGPSVFLLPPKPKDTLM ISRTPEVTCVVVDVSHEDPEVKFNWYVDGVEVHNAKTKPPEEQYNSTLRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPLVLDSDGSFFLYSKLTVDKSRWQQGNVFSCSVMHEALHNHYTQKSLSLSP G (SEQ ID NO: 180); and
a light chain (LC) having the amino acid sequence DWVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(17) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELVGGPSVFLLPPKPKDTLM ISRTPEVTCVVVDVSHEDPEVKFNWYVDGVEVHNAKTKPPEEQYNSTLRVVSVLTVLHQDWLNG KEYKCKVSNWALPAPISKTISKAKGQPREPQVYTLPPSRDELTKNQVSLTCLVKGFYPSDIAVEW ESNGQPENNYKTTPLVLDSDGSFFLYSKLTVDKSRWQQGNVFSCSVMHEALHNHYTQKSLSLS PG (SEQ ID NO: 181); and
a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASWVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(18) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPSVFLF PPKPKDTLMISRTPEVTCVVVDVSHEDPEVKFNWYVDGVEVHNAKTKPREEQYNSTYRVVSVLT VLHQDWLNGKEYKCKVSNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 182); and
a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(19) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVTVPSSSLGTQTYICNVNHKPSNTKVDKKVEPKSCDKTHTCPPCPAPELLGGPDVFLF PPKPKDTLMISRTPEVTCVVDVSHEDPEVKFNWYVDGVEVHNAKTKPREEQYNSTYRVVSVLT VLHQDWLNGKEYKCKVSNKALPAPEEKTISKAKGQPREPQVYTLPPSRDELTKNQVSLTCLVKG FYPSDIAVEWESNGQPENNYKTTPPVLDSDGSFFLYSKLTVDKSRWQQGNVFSCSVMHEALHN HYTQKSLSLSPG (SEQ ID NO: 184); and
a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASWVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(20) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPLPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 185); and
a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(21) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNKALPAPIEKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 186); and
a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(22) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNWALPAPISKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 187); and
a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(23) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPSVFLFPPKPKDTLMI SRTPEVTCVVVDVSHEDPEVKFNWYVDGVEVHNAKTKPREEQYNSTYRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPPVLDSDGSFFLYSKLTVDKSRWQQGNVFSCSVMHEALHNHYTQKSLSLSP G (SEQ ID NO: 188); and
a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
(24) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPDVFLFPPKPKDTLMI SRTPEVTCVVVDVSHEDPEVKFNWYVDGVEVHNAKTKPREEQYNSTYRVVSVLTVLHQDWLNG KEYKCKVSNKALPAPEEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 190); and
a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
(25) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPDVFLFPPKPKDTLMI SRTPEVTCVVVDVSHEDPEVKFNWYVDGVEVHNAKTKPREEQYNSTYRVVSVLTVLHQDWLNG KEYKCKVSNKALPLPEEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 191); and
a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
(26) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELVGGPSVFLLPPKPKDTLMI SRTPEVTCVVVDVSHEDPEVKFNWYVDGVEVHNAKTKPPEEQYNSTLRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPLVLDSDGSFFLYSKLTVDKSRWQQGNVFSCSVMHEALHNHYTQKSLSLSP G (SEQ ID NO: 192); and
a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
(27) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELVGGPSVFLLPPKPKDTLMI SRTPEVTCVVVDVSHEDPEVKFNWYVDGVEVHNAKTKPPEEQYNSTLRVVSVLTVLHQDWLNG KEYKCKVSNWALPAPISKTISKAKGQPREPQVYTLPPSRDELTKNQVSLTCLVKGFYPSDIAVEW ESNGQPENNYKTTPLVLDSDGSFFLYSKLTVDKSRWQQGNVFSCSVMHEALHNHYTQKSLSLS PG (SEQ ID NO: 193); and
a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189).

8. A monoclonal antibody or an antigen-binding fragment thereof, wherein said antibody or antigen-binding fragment specifically binds to human CCR8, and wherein said antibody or antigen-binding fragment comprises:
(1) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23); or
(2) a heavy chain variable domain (VH) comprising a CDR-H1 having the amino acid sequence PYAMN (SEQ ID NO: 6), a CDR-H2 having the amino acid sequence RIRSKSNNYATYYADSVKD (SEQ ID NO: 7), and a CDR-H3 having the amino acid sequence GREAYYRYDGDYYAMDY (SEQ ID NO: 8); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(3) a heavy chain variable domain (VH) comprising a CDR-H1 having the amino acid sequence TYAMN (SEQ ID NO: 12), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(4) a heavy chain variable domain (VH) comprising a CDR-H1 having the amino acid sequence EYTIH (SEQ ID NO: 14), a CDR-H2 having the amino acid sequence GINPNGDTRYDQKFKG (SEQ ID NO: 15), and a CDR-H3 having the amino acid sequence VARFYGISPYAMDY (SEQ ID NO: 16); and
a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQSTHVPYT (SEQ ID NO: 19); or
(5) a heavy chain variable domain (VH) comprising a CDR-H1 having the amino acid sequence AYAMN (SEQ ID NO: 20), a CDR-H2 having the amino acid sequence RIRSKSNDYATYYGDSVKD (SEQ ID NO: 21), and a CDR-H3 having the amino acid sequence GREAYYRYDGGYYAMDY (SEQ ID NO: 13); and
a light chain variable domain (VL) comprising a CDR-L1 having the amino acid sequence TSSKSLLHSNGNTYLY (SEQ ID NO: 22), a CDR-L2 having the amino acid sequence RMSNLAS (SEQ ID NO: 10), and a CDR-L3 having the amino acid sequence MQHREYPFT (SEQ ID NO: 23); or
(6) a heavy chain variable domain (VH) comprising a CDR-H1 having the amino acid sequence DYVVT (SEQ ID NO: 24), a CDR-H2 having the amino acid sequence VIWGGGNTYYNSDLKS (SEQ ID NO: 25), and a CDR-H3 having the amino acid sequence RHRDYALDY (SEQ ID NO: 26); and
a light chain variable domain (VL) comprising a CDR-L1 having the amino acid sequence RSSQSLVYSNGNTYLH (SEQ ID NO: 27), a CDR-L2 having the amino acid sequence KVSNRFS (SEQ ID NO: 18), and a CDR-L3 having the amino acid sequence SQSTYVPPT (SEQ ID NO: 28); or
(7) a heavy chain variable domain (VH) comprising a CDR-H1 having the amino acid sequence DYTLH (SEQ ID NO: 29), a CDR-H2 having the amino acid sequence GITPKNGDTRYDPRFKD (SEQ ID NO: 30), and a CDR-H3 having the amino acid sequence VARFYGVSPYAMDY (SEQ ID NO: 31); and
a light chain variable domain (VL) comprising a CDR-L1 having the amino acid sequence RCTQSLLHSNGDTYLH (SEQ ID NO: 17), a CDR-L2 having the amino acid sequence KVSNRFS (SEQ ID NO: 18), and a CDR-L3 having the amino acid sequence SQTTHVPYT (SEQ ID NO: 32); or
(8) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PDGFSPFVY (SEQ ID NO: 35); and
a light chain variable domain (VL) comprising a CDR-L1 having the amino acid sequence KASQDINSYLS (SEQ ID NO: 36), a CDR-L2 having the amino acid sequence RANRLVD (SEQ ID NO: 37), and a CDR-L3 having the amino acid sequence LQYGEFPPT (SEQ ID NO: 38); or
(9) a heavy chain variable domain (VH) comprising a CDR-H1 having the amino acid sequence AYAMN (SEQ ID NO: 20), a CDR-H2 having the amino acid sequence RIRSKSNNYATYYGDSVKD (SEQ ID NO: 39), and a CDR-H3 having the amino acid sequence GREAYYRYDGDYYAMDY (SEQ ID NO: 8); and
a light chain variable domain (VL) comprising a CDR-L1 having the amino acid sequence KSSQSLLNSSNQKNYLA (SEQ ID NO: 40), a CDR-L2 having the amino acid sequence FASTRES (SEQ ID NO: 41), and a CDR-L3 having the amino acid sequence QQHYSTPYT (SEQ ID NO: 42); or
(10) a heavy chain variable domain (VH) comprising a CDR-H1 having the amino acid sequence AYAMN (SEQ ID NO: 20), a CDR-H2 having the amino acid sequence RIRSKSNNYATYYADSVKD (SEQ ID NO: 7), and a CDR-H3 having the amino acid sequence GREAYYRYDGGYYAMDY (SEQ ID NO: 13); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(11) a heavy chain variable domain (VH) comprising a CDR-H1 having the amino acid sequence EYTIH (SEQ ID NO: 14), a CDR-H2 having the amino acid sequence GINPNNGNTRYDQKFKG (SEQ ID NO: 43), and a CDR-H3 having the amino acid sequence VARSSGSGPYAMDY (SEQ ID NO: 44); and
a light chain variable domain (VL) comprising a CDR-L1 having the amino acid sequence RCTQSLLHTNGDTYLH (SEQ ID NO: 45), a CDR-L2 having the amino acid sequence KVSNRFS (SEQ ID NO: 18), and a CDR-L3 having the amino acid sequence SQSTHVPYT (SEQ ID NO: 19); or
(12) a heavy chain variable domain (VH) comprising a CDR-H1 having the amino acid sequence GYNMH (SEQ ID NO: 46), a CDR-H2 having the amino acid sequence AISPGKGDTSYNLKFKG (SEQ ID NO: 47), and a CDR-H3 having the amino acid sequence SGGTPFAY (SEQ ID NO: 48); and
a light chain variable domain (VL) comprising a CDR-L1 having the amino acid sequence RSSQSLVHSNGDTYLY (SEQ ID NO: 49), a CDR-L2 having the amino acid sequence KVSNRFS (SEQ ID NO: 18), and a CDR-L3 having the amino acid sequence SQSTHVPYT (SEQ ID NO: 19); or
(13) a heavy chain variable domain (VH) comprising a CDR-H1 having the amino acid sequence ANAMN (SEQ ID NO: 50), a CDR-H2 having the amino acid sequence RIRSKSNNYATYYADSVKD (SEQ ID NO: 7), and a CDR-H3 having the amino acid sequence GSDNYIFYAMDY (SEQ ID NO: 51); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(14) a heavy chain variable domain (VH) comprising a CDR-H1 having the amino acid sequence TNAMN (SEQ ID NO: 52), a CDR-H2 having the amino acid sequence RIRSKSNYYATYYADSVKD (SEQ ID NO: 53), and a CDR-H3 having the amino acid sequence GREMGNYYSMDY (SEQ ID NO: 54); and
a light chain variable domain (VL) comprising a CDR-L1 having the amino acid sequence RSSKSLLHSNGNTYLY (SEQ ID NO: 9), a CDR-L2 having the amino acid sequence RMSNLAS (SEQ ID NO: 10), and a CDR-L3 having the amino acid sequence MQHLEYPFT (SEQ ID NO: 11); or
(15) a heavy chain variable domain (VH) comprising a CDR-H1 having the amino acid sequence NYRMH (SEQ ID NO: 33), a CDR-H2 having the amino acid sequence VIKVKSDNYGANYAESVKG (SEQ ID NO: 34), and a CDR-H3 having the amino acid sequence PTYPGSSGFAY (SEQ ID NO: 55); and
a light chain variable domain (VL) comprising a CDR-L1 having the amino acid sequence RASQDISNYLN (SEQ ID NO: 56), a CDR-L2 having the amino acid sequence YTSRLHS (SEQ ID NO: 57), and a CDR-L3 having the amino acid sequence QQGNKFPPT (SEQ ID NO: 58); or
(16) a heavy chain variable domain (VH) comprising a CDR-H1 having the amino acid sequence EYTMH (SEQ ID NO: 59), a CDR-H2 having the amino acid sequence GINPNNGDTNYNQKFMG (SEQ ID NO: 60), and a CDR-H3 having the amino acid sequence RLLRRGAMDY (SEQ ID NO: 61); and
a light chain variable domain (VL) comprising a CDR-L1 having the amino acid sequence RASENVEYYGTSLMQ (SEQ ID NO: 62), a CDR-L2 having the amino acid sequence AASNVDS (SEQ ID NO: 63), and a CDR-L3 having the amino acid sequence QQSRKVPWT (SEQ ID NO: 64); or
(17) a heavy chain variable domain (VH) comprising a CDR-H1 having the amino acid sequence GSYMH (SEQ ID NO: 65), a CDR-H2 having the amino acid sequence RINPYNGATSYNQNFKD (SEQ ID NO: 66), and a CDR-H3 having the amino acid sequence TLLRLLDY (SEQ ID NO: 67); and
a light chain variable domain (VL) comprising a CDR-L1 having the amino acid sequence RSSKSLLHSNGITYLY (SEQ ID NO: 68), a CDR-L2 having the amino acid sequence QMSNLAS (SEQ ID NO: 69), and a CDR-L3 having the amino acid sequence AQNLELPWT (SEQ ID NO: 70); or
(18) a heavy chain variable domain (VH) comprising a CDR-H1 having the amino acid sequence SYWMH (SEQ ID NO: 71), a CDR-H2 having the amino acid sequence NIWPGSASTNYDEKFKN (SEQ ID NO: 72), and a CDR-H3 having the amino acid sequence GGKGAMDY (SEQ ID NO: 73); and
a light chain variable domain (VL) comprising a CDR-L1 having the amino acid sequence RSSQSLENSYGNTYLN (SEQ ID NO: 74), a CDR-L2 having the amino acid sequence RVSNRFS (SEQ ID NO: 75), and a CDR-L3 having the amino acid sequence LQVTHVPPT (SEQ ID NO: 76); or
(19) a heavy chain variable domain (VH) comprising a CDR-H1 having the amino acid sequence TYAMH (SEQ ID NO: 77), a CDR-H2 having the amino acid sequence HINPSSGYSNYNQKFKD (SEQ ID NO: 78), and a CDR-H3 having the amino acid sequence SEVRRGYFDV (SEQ ID NO: 79); and
a light chain variable domain (VL) comprising a CDR-L1 having the amino acid sequence RTSQDIRNYLN (SEQ ID NO: 80), a CDR-L2 having the amino acid sequence YTSRLHS (SEQ ID NO: 57), and a CDR-L3 having the amino acid sequence QQGNTLPPT (SEQ ID NO: 81); or
(20) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDV (SEQ ID NO: 124); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11); or
(21) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDV (SEQ ID NO: 124); and
a light chain variable domain (VL) comprising a CDR-L1 having the amino acid sequence KSLLHSNGNTY (SEQ ID NO: 125), a CDR-L2 having the amino acid sequence RVS, and a CDR-L3 having the amino acid sequence MQHREYPFT (SEQ ID NO: 23); or
(22) a heavy chain variable domain (VH) comprising a CDR-H1 having the amino acid sequence GYTFTEYT (SEQ ID NO: 128), a CDR-H2 having the amino acid sequence INPNNGNT (SEQ ID NO: 129), and a CDR-H3 having the amino acid sequence ARVARSSGSGPYAMDY (SEQ ID NO: 130); and
a light chain variable domain (VL) comprising a CDR-L1 having the amino acid sequence QSLLHTNGDTY (SEQ ID NO: 131), a CDR-L2 having the amino acid sequence KVS, and a CDR-L3 having the amino acid sequence SQSTHVPYT (SEQ ID NO: 19); or
(23) a heavy chain variable domain (VH) comprising a CDR-H1 having the amino acid sequence GFTFNAYA (SEQ ID NO: 126), a CDR-H2 having the amino acid sequence IRSKSNDYAT (SEQ ID NO: 127), and a CDR-H3 having the amino acid sequence VRGREAYYRYDGGYYAMDY (SEQ ID NO: 133); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHREYPFT (SEQ ID NO: 23); or
(24) a heavy chain variable domain (VH) comprising a CDR-H1 having the amino acid sequence GFTFNTYA (SEQ ID NO: 122), a CDR-H2 having the amino acid sequence IRSKSNNYAT (SEQ ID NO: 123), and a CDR-H3 having the amino acid sequence VRGREAYYRYDGGYYAMDY (SEQ ID NO: 133); and
a light chain variable domain (VL) comprising a CDR-L1 having the amino acid sequence KSLLHSNGNTY (SEQ ID NO: 125), a CDR-L2 having the amino acid sequence RMS, and a CDR-L3 having the amino acid sequence MQHLEYPFT (SEQ ID NO: 11); or
(25) a heavy chain variable domain (VH) comprising a CDR-H1 having the amino acid sequence GFTFSNYR (SEQ ID NO: 134), a CDR-H2 having the amino acid sequence IKVKSDNYGA (SEQ ID NO: 135), and a CDR-H3 having the amino acid sequence SSPTYPGSSGFAY (SEQ ID NO: 136); and
a light chain variable domain (VL) comprising a CDR-L1 having the amino acid sequence QDISNY (SEQ ID NO: 137), a CDR-L2 having the amino acid sequence YTS, and a CDR-L3 having the amino acid sequence QQGNKFPPT (SEQ ID NO: 58).

9. A monoclonal antibody or an antigen-binding fragment thereof, wherein said antibody or antigen-binding fragment specifically binds to human CCR8, and wherein said antibody or antigen-binding fragment comprises:
(1) a heavy chain variable domain (VH) having an amino acid sequence with at least 80% sequence identity to the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SS (SEQ ID NO: 140); and
a light chain variable domain (VL) having an amino acid sequence with at least 80% sequence identity to the amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIK (SEQ ID NO: 141); or
(2) a heavy chain variable domain (VH) having an amino acid sequence with at least 80% sequence identity to the amino acid sequence MLLGLKWVFFVVFYQGVLCEVQLLESGGGLVQPKGSLKLSCAASGFTFNPYAMNWVRQAPGRG LEWVARIRSKSNNYATYYADSVKDRFTISRDDSQDMLYLQMNNLKTEDTAMYYCVRGREAYYRY DGDYYAMDYWGQGTSVTVSS (SEQ ID NO: 82); and
a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
(3) a heavy chain variable domain (VH) having an amino acid sequence with at least 80% sequence identity to the amino acid sequence MLLGLKWVFFVVFYQGVHCEVQLVESGGGLVQPKGSLKLSCAASGFTFNTYAMNWVRQAPGK GLEWVARIRSKSNNYATYYADSVKDRFTISRDDSQSMLYLQMNNLKTEDTAMYYCVRGREAYYR YDGGYYAMDYWGQGTSVTVSS (SEQ ID NO: 84); and
a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
(4) a heavy chain variable domain (VH) having an amino acid sequence with at least 80% sequence identity to the amino acid sequence MGWSWIFLFLLSGTAGVLSKVQLQQSGPELVKPGASVKISCKTSGYTFTEYTIHWVQQIHGKSPE WIGGINPNGDTRYDQKFKGKATLTIDKSSSTAYMELRSLTSEDSAVYYCARVARFYGISPYAMDY WGQGTSVTVSS (SEQ ID NO: 85); and
a light chain variable domain (VL) having an amino acid sequence with at least 80% sequence identity to the amino acid sequence MKLPVRLLVLMFWIPASSSDVVMTQTPLSLPVSLGDQASISCRCTQSLLHSNGDTYLHWYLQKP GQSPNLLIYKVSNRFSGVPDRFSGSGSGTDFTLKINRVEAEDLGVYFCSQSTHVPYTFGGGTNLE IK (SEQ ID NO: 86); or
(5) a heavy chain variable domain (VH) having an amino acid sequence with at least 80% sequence identity to the amino acid sequence MLLGLKWVFFVVFYQGVHCEVQLVESGGGLVQPTGSLRLSCAASGFTFNAYAMNWVRQAPGK GLEWVARIRSKSNDYATYYGDSVKDRFTISRDDSQSMLYLQMNNLKTEDTAMYYCVRGREAYY RYDGGYYAMDYWGQGTSVTVSS (SEQ ID NO: 87); and
a light chain variable domain (VL) having an amino acid sequence with at least 80% sequence identity to the amino acid sequence MRCLAEFLGLLVLWIPGAIGDIVMTQAAPSVSVTPGESVSISCTSSKSLLHSNGNTYLYWFLQRPG QSPQLLIYRMSNLASGVPDRFSGSGSGTAFTLRISRVEAEDVGVYYCMQHREYPFTFGSGTKLEI K (SEQ ID NO: 88); or
(6) a heavy chain variable domain (VH) having an amino acid sequence with at least 80% sequence identity to the amino acid sequence MAVLGLLLCLVTFPSCVLSQVQLKESGPGLVAPSQSLSITCTVSGLSMNDYVVTWIRQPPGKGLE WLGVIWGGGNTYYNSDLKSRLSITKDNSKSQVFFKMSSLQTDDTAVYYCARRHRDYALDYWGQ GISVTVSS (SEQ ID NO: 89); and
a light chain variable domain (VL) having an amino acid sequence with at least 80% sequence identity to the amino acid sequence MKLPVRLLVLMFWIPASSSDVVMTQTPLSLPVSLGDQASISCRSSQSLVYSNGNTYLHWYLQKP GQSPKLLIYKVSNRFSGVPDRFSGSGSGTDFTLKISRVEAEDLGVYFCSQSTYVPPTFGGGTKLEI K (SEQ ID NO: 90); or
(7) a heavy chain variable domain (VH) having an amino acid sequence with at least 80% sequence identity to the amino acid sequence MGWSWIFLFLLSGTAGVLSEVQLQQSGPELLKPGTSVKISCTTSGYTFSDYTLHWVKQSHGKSL EWIGGITPKNGDTRYDPRFKDKATLTIDKSSSAAYMELRSLTSEDSAVYYCARVARFYGVSPYAM DYWGQGASVTVSS (SEQ ID NO: 91); and
a light chain variable domain (VL) having an amino acid sequence with at least 80% sequence identity to the amino acid sequence MKLPVRLLVLMFWIPVSNSDVVMTQTPLSLPVSLGDQASISCRCTQSLLHSNGDTYLHWYLQKP GQSPKLLIYKVSNRFSGVPDRFSGSGSGTDFTLEITRVEAEDLGVYFCSQTTHVPYTFGGGTKLEI K (SEQ ID NO: 92); or
(8) a heavy chain variable domain (VH) having an amino acid sequence with at least 80% sequence identity to the amino acid sequence MELGLSWVFLVALLNGVQCQVHLVETGGGLVRPGNSLKLSCVTSGFTLSNYRMHWLRQPPGKR LEWIAVIKVKSDNYGANYAESVKGRFTISRDDAKSSVYLQMNRLREEDTATYYCSRPDGFSPFVY WGQGTLVTVSA (SEQ ID NO: 93); and
a light chain variable domain (VL) having an amino acid sequence with at least 80% sequence identity to the amino acid sequence MDMRTPAQFLGILLLWFPGIKCDIKMTQSPSSMYASLGERVTITCKASQDINSYLSWFQQKPGKS PKTLIYRANRLVDGVPSRFSGSGSGQDYFLTISSLEYEDMGIYYCLQYGEFPPTFGAGTKLELK (SEQ ID NO: 94); or
(9) a heavy chain variable domain (VH) having an amino acid sequence with at least 80% sequence identity to the amino acid sequence MLLGLKWVFFVVFYQGVHCEVQLVESGGGLVQPKGSLRLSCAASGFTFNAYAMNWVRQAPGK GLEWVARIRSKSNNYATYYGDSVKDRFTISRDDSQTMLYLQMNNLKTEDTAMYYCVRGREAYYR YDGDYYAMDYWGQGTSVTVSS (SEQ ID NO: 95); and
a light chain variable domain (VL) having an amino acid sequence with at least 80% sequence identity to the amino acid sequence MESQTQVLMFLLLWVSGACADIVMTQSPSSLAMSVGQKVTMSCKSSQSLLNSSNQKNYLAWYQ QKPGQSPKLLVYFASTRESGVPDRFIGSGSGTDFTLTISSVQAEDLADYFCQQHYSTPYTFGGGT KLEIK (SEQ ID NO: 96); or
(10) a heavy chain variable domain (VH) having an amino acid sequence with at least 80% sequence identity to the amino acid sequence MLLGLKWVFFVVFYQGVHCEVQLVESGGGLVQPKGSLKLSCAASGFTFNAYAMNWVRQAPGK GLEWLARIRSKSNNYATYYADSVKDRFTISRDDSQSMFYLQMNNLKSEDTAMYYCVRGREAYYR YDGGYYAMDYWGQGTSVTVSS (SEQ ID NO: 97); and
a light chain variable domain (VL) having an amino acid sequence with at least 80% sequence identity to the amino acid sequence MRCLAEFLGLLVLWIPGAIGEIVMTQAALSAPVTPGESVSMSCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 98); or
(11) a heavy chain variable domain (VH) having an amino acid sequence with at least 80% sequence identity to the amino acid sequence MGWSWIFLFLLSGTAGVLSEVQLQQSGPELVKPGASVKISCKTSGYTFTEYTIHWVKQSHGESLE WVGGINPNNGNTRYDQKFKGKATLTIDKSSSPAYMELRSLTSEDSAVYYCARVARSSGSGPYAM DYWGQGTSVTVSS (SEQ ID NO: 99); and
a light chain variable domain (VL) having an amino acid sequence with at least 80% sequence identity to the amino acid sequence MKLPVRLLVLMFWIPVSSSDVVMTQTPLSLPVSLGDQASISCRCTQSLLHTNGDTYLHWYLQKPG QSPKLLIYKVSNRFSGVPDRFSGSGSGTDFTLKISRVEAEDLGVYFCSQSTHVPYTFGGGTKLEIK (SEQ ID NO: 100); or
(12) a heavy chain variable domain (VH) having an amino acid sequence with at least 80% sequence identity to the amino acid sequence MGWSCIILFLVATATDVHSQVQLQQPGAELVKPGASVKMSCKATGYTFTGYNMHWVKQTPGQG LEWIGAISPGKGDTSYNLKFKGKATLTTDKSSSTAYMQLSSLTSADSAVYYCARSGGTPFAYWG QGTLVTVSA (SEQ ID NO: 101); and
a light chain variable domain (VL) having an amino acid sequence with at least 80% sequence identity to the amino acid sequence MKLPVRLLVLMFWIPASSSDVVMTQTPLSLPVSLGDQASISCRSSQSLVHSNGDTYLYWYLQKP GQSPKLLIYKVSNRFSGVPDRFSGSGSGTDFTLKINRVEAEDLGVYFCSQSTHVPYTFGGGTKLE IK (SEQ ID NO: 102); or
(13) a heavy chain variable domain (VH) having an amino acid sequence with at least 80% sequence identity to the amino acid sequence MLLGLKWVFFVVFYQGVHCEVQLVETGGGLVQPKGSLKLSCAASGFTFNANAMNWVRQAPGK GLEWVARIRSKSNNYATYYADSVKDRFTISRDDSQSMLYLQMNNLKTEDTAMYYCVRGSDNYIF YAMDYWGQGTSVTVSS (SEQ ID NO: 103); and
a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGGGTKL EIK (SEQ ID NO: 104); or
(14) a heavy chain variable domain (VH) having an amino acid sequence with at least 80% sequence identity to the amino acid sequence MLLGLKWVFFVVFYQGVHCEVQLVETGGGLVQPKGSLKLSCAASGFTFNTNAMNWVRQAPGK GLEWVARIRSKSNYYATYYADSVKDRFTISRDDSQSMLYLQMNNLKTEDTAMYYCVRGREMGN YYSMDYWGQGTSVTVSS (SEQ ID NO: 105); and
a light chain variable domain (VL) having an amino acid sequence with at least 80% sequence identity to the amino acid sequence MRCLAEFLGLLVLWIPGAIGDIVMTQAAPSVPVTPGESVSISCRSSKSLLHSNGNTYLYWFLQRP GQSPQLLIYRMSNLASGVPDRFSGSGSGTAFTLRISRVEAEDVGVYYCMQHLEYPFTFGSGTKL EIK (SEQ ID NO: 83); or
(15) a heavy chain variable domain (VH) having an amino acid sequence with at least 80% sequence identity amino to the acid sequence MELGLSWVFLVALLNGVQCQVQLVETGGGLVRPGNSLKLSCVTSGFTFSNYRMHWLRQPPGKR LEWIAVIKVKSDNYGANYAESVKGRFTISRDDSKSSVYLQVNRLREEDTATYYCSSPTYPGSSGF AYWGQGTLVTVSA (SEQ ID NO: 106); and
a light chain variable domain (VL) having an amino acid sequence with at least 80% sequence identity to the amino acid sequence MMSSAQFLGLLLLCFQGTRCDIQMTQTTSSLSASLGDRVTISCRASQDISNYLNWYQQKPDGTV KLLIYYTSRLHSGVPSRFSGSGSGTDYSLTISNLEQEDIATYFCQQGNKFPPTFGGGTKLEIN (SEQ ID NO: 107); or
(16) a heavy chain variable domain (VH) having an amino acid sequence with at least 80% sequence identity to the amino acid sequence MGWSWIFLFLLSGTAGVLSEVQLQQSGPELVKPGASVKISCKTSGYTFTEYTMHWVKQSHGKSL EWIGGINPNNGDTNYNQKFMGKATLTVDKSSSTAYMELRSLTSEDSAVYYCVRRLLRRGAMDY WGQGTSVTVSS (SEQ ID NO: 108); and
a light chain variable domain (VL) having an amino acid sequence with at least 80% sequence identity to the amino acid sequence MESDTLLLWVLLLWVPGSTGDIVLTQSPASLAVSLGQRATISCRASENVEYYGTSLMQWFQQKP GQPPKLLIYAASNVDSGVPARFSGSGSGTDFSLNIHPVEEDDIAMYFCQQSRKVPWTFGGGTKL DIK (SEQ ID NO: 109); or
(17) a heavy chain variable domain (VH) having an amino acid sequence with at least 80% sequence identity to the amino acid sequence MGWSWIFLFLLSGTAGVLSEVQLQQSGPELVKPGASVKISCKASVYSFTGSYMHWVKQSHVKSL EWIGRINPYNGATSYNQNFKDKASLTVDKSSSTAYMELHSLTSEDSAVYYCATTLLRLLDYWGQ GTTLTVSS (SEQ ID NO: 110); and
a light chain variable domain (VL) having an amino acid sequence with at least 80% sequence identity to the amino acid sequence MRFSAQLLGLLVLWIPGSTADIVMTQAAFSNPVTLGTSASISCRSSKSLLHSNGITYLYWYLQKPG QSPQLLIYQMSNLASGVPDRESSSGSGTDFTLRISRVEAEDVGVYYCAQNLELPWTFGGGTKLEI K (SEQ ID NO: 111); or
(18) a heavy chain variable domain (VH) having an amino acid sequence with at least 80% sequence identity to the amino acid sequence MGWSSIILFLVATASGVHSQVQLQQPGSELVRPGASVKLSCKASGYTFTSYWMHWVKQRPGQG LEWIGNIWPGSASTNYDEKFKNKATLTVDTSSSTAYMQLSSLTSEDSAVYYCIRGGKGAMDYWS QGTSVTVSS (SEQ ID NO: 112); and
a light chain variable domain (VL) having an amino acid sequence with at least 80% sequence identity to the amino acid sequence MKLPVRLLVLMFWIPVSSSDVVMTQIPLSLPVSLGDQASISCRSSQSLENSYGNTYLNWYLQKPG QSPQLLIYRVSNRFSGVLDRFSGSGSGTDFTLKISRVEAEDLGVYFCLQVTHVPPTFGAGTKLEL K (SEQ ID NO: 113); or
(19) a heavy chain variable domain (VH) having an amino acid sequence with at least 80% sequence identity to the amino acid sequence MERHWIFLLLLSVTAGVHSQVQVQQSGAELARPGASVKMSCKASGYTFTTYAMHWVKQRPGQ GLEWIGHINPSSGYSNYNQKFKDKATLTADKSSSTAYMQLSSLTSEDSAVYYCARSEVRRGYFD VWGAGTTVTVSS (SEQ ID NO: 114); and
a light chain variable domain (VL) having an amino acid sequence with at least 80% sequence identity to the amino acid sequence MMSSAQFLGLLLLCFQGTRCDIQMTQTTSSLSASLGDRVTISCRTSQDIRNYLNWYQQKPDGTV KLLISYTSRLHSGVPSRFSGSGSGTDYSLTISNLEQEDFATYFCQQGNTLPPTFGGGTRVEIK (SEQ ID NO: 115); or
(20) a heavy chain variable domain (VH) having an amino acid sequence with at least 80% sequence identity to the amino acid sequence EVOLVESGGGLVQPGRSLRLSCTASGFTFNTYAMNWVRQAPGKGLEWVARIRSKSNNYATYYA ASVKGRFTISRDDSKSIAYLQMNSLKTEDTAVYYCVRGREAYYRYDGGYYAMDVWGQGTTVTV SS (SEQ ID NO: 138); and
a light chain variable domain (VL) having an amino acid sequence with at least 80% sequence identity to the amino acid sequence DIVMTQAAPSLPVTPGESASISCRSSKSLLHSNGNTYLYWFLQKPGQSPQLLIYRMSNLASGVPD RFSGSGSGTAFTLKISRVEAEDVGVYYCMQHLEYPFTFGSGTKLEIK (SEQ ID NO: 139); or
(21) a heavy chain variable domain (VH) having an amino acid sequence with at least 80% sequence identity to the acid amino sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SS (SEQ ID NO: 140); and
a light chain variable domain (VL) having an amino acid sequence with at least 80% sequence identity to the amino acid sequence DIVMTQSPLSLPVTPGEPASISCRSSKSLLHSNGNTYLYWFLQKPGQSPQLLIYRVSNLASGVPD RFSGSGSGTDFTLKISRVEAEDVGVYYCMQHREYPFTFGQGTKLEIK (SEQ ID NO: 142); or
(22) a heavy chain variable domain (VH) having an amino acid sequence with at least 80% sequence identity to the amino acid sequence QVQLVQSGPEVVKPGASVKVSCKTSGYTFTEYTIHWVRQAPGQSLEWVGGINPNNGNTRYDQK FQGRVTITRDKSASTAYMELRSLTSEDTAVYYCARVARSSGSGPYAMDYWGQGTTVTVSS (SEQ ID NO: 143); and
a light chain variable domain (VL) having an amino acid sequence with at least 80% sequence identity to the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIK (SEQ ID NO: 144); or
(23) a heavy chain variable domain (VH) having an amino acid sequence with at least 80% sequence identity to the amino acid sequence QVQLQQSGPEVVKPGASVKVSCKTSGYTFTEYTIHWVRQSHGQSLEWVGGINPNNGNTRYDQK FKGRVTITIDKSSSTAYMELRSLTSEDTAVYYCARVARSSGSGPYAMDYWGQGTTVTVSS (SEQ ID NO: 145); and
a light chain variable domain (VL) having an amino acid sequence with at least 80% sequence identity to the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIK (SEQ ID NO: 144); or
(24) a heavy chain variable domain (VH) having an amino acid sequence with at least 80% sequence identity to the amino acid sequence QVQLVESGGGVVQPGRSLRLSCAASGFTFSNYRMHWVRQAPGKGLEWIAVIKVKSDNYGANYA DSVKGRFTISRDNSKNTLYLQMNSLRAEDTAVYYCSSPTYPGSSGFAYWGQGTLVTVSS (SEQ ID NO: 146); and
a light chain variable domain (VL) having an amino acid sequence with at least 80% sequence identity to the amino acid sequence DIQMTQSPSSLSASVGDRVTITCRASQDISNYLNWYQQKPDGTVKLLIYYTSRLHTGVPSRFSGS GSGTDYTLTISNLQQEDIATYFCQQGNKFPPTFGGGTKVEIN (SEQ ID NO: 132); or
(25) a heavy chain variable domain (VH) having an amino acid sequence with at least 80% sequence identity to the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSS (SEQ ID NO: 147); and
a light chain variable domain (VL) having an amino acid sequence with at least 80% sequence identity to the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIK (SEQ ID NO: 148); or
(26) a heavy chain variable domain (VH) having an amino acid sequence with at least 80% sequence identity to the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSS (SEQ ID NO: 149); and
a light chain variable domain (VL) having an amino acid sequence with at least 80% sequence identity to the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIK (SEQ ID NO: 150); or
(27) a heavy chain variable domain (VH) having an amino acid sequence with at least 80% sequence identity to the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSS (SEQ ID NO: 151); and
a light chain variable domain (VL) having an amino acid sequence with at least 80% sequence identity to the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIK (SEQ ID NO: 152); or
(28) a heavy chain variable domain (VH) having an amino acid sequence with at least 80% sequence identity to the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSA (SEQ ID NO: 153); and
a light chain variable domain (VL) having an amino acid sequence with at least 80% sequence identity to the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEIN (SEQ ID NO: 154).

10. A monoclonal antibody or an antigen-binding fragment thereof, wherein said antibody or antigen-binding fragment specifically binds to human CCR8, and wherein said antibody or antigen-binding fragment comprises:
(1) a heavy chain (HC) having the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVTVPSSSLGTQTYICNVNHKPSNTKVDKKVEPKSCDKTHTCPPCPAPELLGGPDVFLFP PKPKDTLMISRTPEVTCVVVDVSHEDPEVKFNWYVDGVEVHNAKTKPREEQYNSTYRVVSVLTV LHQDWLNGKEYKCKVSNKALPAPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 163); and
a light chain (LC) having the amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 162); or
(2) a heavy chain (HC) having the amino acid sequence EVOLVESGGGLVQPGRSLRLSCTASGFTFNTYAMNWVRQAPGKGLEWVARIRSKSNNYATYYA ASVKGRFTISRDDSKSIAYLQMNSLKTEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSWVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 159); and
a light chain (LC) having the amino acid sequence DIVMTQAAPSLPVTPGESASISCRSSKSLLHSNGNTYLYWFLQKPGQSPQLLIYRMSNLASGVPD RFSGSGSGTAFTLKISRVEAEDVGVYYCMQHLEYPFTFGSGTKLEIKRTVAAPSVFIFPPSDEQLK SGTASWVCLLNNFYPREAKVQWKVDNALQSGNSQESVTEQDSKDSTYSLSSTLTLSKADYEKHK VYACEVTHQGLSSPVTKSFNRGEC (SEQ ID NO: 160); or
(3) a heavy chain (HC) having the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 161); and
a light chain (LC) having the amino acid sequence DIVMTQAAPSLSVTPGESASISCTSSKSLLHSNGNTYLYWFLQKPGQSPQLLIYRMSNLASGVPD RFSGSGSGTAFTLKISRVEAEDVGVYYCMQHREYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 162); or
(4) a heavy chain (HC) having the amino acid sequence EVQLLESGGGLVQPGGSLRLSCAASGFTFNAYAMNWVRQAPGKGLEWVARIRSKSNDYATYYG DSVKGRFTISRDNSKNTLYLQMNSLRAEDTAVYYCVRGREAYYRYDGGYYAMDVWGQGTTVTV SSASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEALHNHY TQKSLSLSPG (SEQ ID NO: 161); and
a light chain (LC) having the amino acid sequence DIVMTQSPLSLPVTPGEPASISCRSSKSLLHSNGNTYLYWFLQKPGQSPQLLIYRVSNLASGVPD RFSGSGSGTDFTLKISRVEAEDVGVYYCMQHREYPFTFGQGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 164); or
(5) a heavy chain (HC) having the amino acid sequence QVQLVQSGPEVVKPGASVKVSCKTSGYTFTEYTIHWVRQAPGQSLEWVGGINPNNGNTRYDQK FQGRVTITRDKSASTAYMELRSLTSEDTAVYYCARVARSSGSGPYAMDYWGQGTTVTVSSASTK GPSVFPLAPSSKSTSGGTAALGCLVKDYFPEPVTVSWNSGALTSGVHTFPAVLQSSGLYSLSSV VTVPSSSLGTQTYICNVNHKPSNTKVDKKVEPKSCDKTHTCPPCPAPELLGGPSVFLFPPKPKDT LMISRTPEVTCVVVDVSHEDPEVKFNWYVDGVEVHNAKTKPREEQYNSTYRVVSVLTVLHQDWL NGKEYKCKVSNKALPAPIEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 165); and
a light chain (LC) having the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO: 166); or
(6) a heavy chain (HC) having the amino acid sequence QVQLQQSGPEVVKPGASVKVSCKTSGYTFTEYTIHWVRQSHGQSLEWVGGINPNNGNTRYDQK FKGRVTITIDKSSSTAYMELRSLTSEDTAVYYCARVARSSGSGPYAMDYWGQGTTVTVSSASTK GPSVFPLAPSSKSTSGGTAALGCLVKDYFPEPVTVSWNSGALTSGVHTFPAVLQSSGLYSLSSV VTVPSSSLGTQTYICNVNHKPSNTKVDKKVEPKSCDKTHTCPPCPAPELLGGPSVFLFPPKPKDT LMISRTPEVTCVVVDVSHEDPEVKFNWYVDGVEVHNAKTKPREEQYNSTYRVVSVLTVLHQDWL NGKEYKCKVSNKALPAPIEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 167); and
a ight chain (LC) having the amino acid sequence DVVMTQSPLSLPVTLGDQASISCRCSQSLLHTNGDTYLHWYLQRPGQSPRLLIYKVSNRFSGVP DRFSGSGSGTDFTLKISRVEAEDVGVYFCSQSTHVPYTFGGGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO: 166); or
(7) a heavy chain (HC) having the amino acid sequence QVQLVESGGGVVQPGRSLRLSCAASGFTFSNYRMHWVRQAPGKGLEWIAVIKVKSDNYGANYA DSVKGRFTISRDNSKNTLYLQMNSLRAEDTAVYYCSSPTYPGSSGFAYWGQGTLVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPSVFLFPPKPKDTLM ISRTPEVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 168); and
a light chain (LC) having the amino acid sequence DIQMTQSPSSLSASVGDRVTITCRASQDISNYLNWYQQKPDGTVKLLIYYTSRLHTGVPSRFSGS GSGTDYTLTISNLQQEDIATYFCQQGNKFPPTFGGGTKVEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 169); or
(8) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPSVFLF PPKPKDTLMISRTPEVTCVVVDVSHEDPEVKFNWYVDGVEVHNAKTKPREEQYNSTYRVVSVLT VLHQDWLNGKEYKCKVSNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 170); and
a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(9) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPAPEEKTISKAKGQPREPQVYTLPPSRDELTKNQVSLTCLVKG FYPSDIAVEWESNGQPENNYKTTPPVLDSDGSFFLYSKLTVDKSRWQQGNVFSCSVMHEALHN HYTQKSLSLSPG (SEQ ID NO: 172); and
a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASWVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(10) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPLPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 173); and
a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(11) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNKALPAPIEKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 174); and
a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(12) a heavy chain (HC) having the amino acid sequence EVOLVESGGGLVQPTGSLRLSCAASGFTFNAYAMNWVRQAPGKGLEWVARIRSKSNDYATYYG DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNWALPAPISKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 175); and
a light chain (LC) having the amino acid sequence DIVMTQAAPSVSVTPGESVSISCTSSKSLLHSNGNTYLYWFLQRPGQSPQLLIYRMSNLASGVPD RFSGSGSGTAFTLRISRVEAEDVGVYYCMQHREYPFTFGSGTKLEIKRTVAAPSVFIFPPSDEQL KSGTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 171); or
(13) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPSVFLFPPKPKDTLM ISRTPEVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 176); and
a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(14) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPDVFLFPPKPKDTLM ISRTPEVTCVVVDVSHEDPEVKFNWYVDGVEVHNAKTKPREEQYNSTYRVVSVLTVLHQDWLN GKEYKCKVSNKALPAPEEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 178); and
a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(15) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELLGGPDVFLFPPKPKDTLM ISRTPEVTCVVVDVSHEDPEVKFNWYVDGVEVHNAKTKPREEQYNSTYRVVSVLTVLHQDWLN GKEYKCKVSNKALPLPEEKTISKAKGQPREPQVYTLPPSRDELTKNQVSLTCLVKGFYPSDIAVE WESNGQPENNYKTTPPVLDSDGSFFLYSKLTVDKSRWQQGNVFSCSVMHEALHNHYTQKSLSL SPG (SEQ ID NO: 179); and
a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(16) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELVGGPSVFLLPPKPKDTLM ISRTPEVTCVVVDVSHEDPEVKFNWYVDGVEVHNAKTKPPEEQYNSTLRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPLVLDSDGSFFLYSKLTVDKSRWQQGNVFSCSVMHEALHNHYTQKSLSLSP G (SEQ ID NO: 180); and
a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(17) a heavy chain (HC) having the amino acid sequence EVQLQQSGPELVKPGASVKISCKTSGYTFTEYTIHWVKQSHGESLEWVGGINPNNGNTRYDQKF KGKATLTIDKSSSPAYMELRSLTSEDSAVYYCARVARSSGSGPYAMDYWGQGTSVTVSSASTKG PSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTCPPCPAPELVGGPSVFLLPPKPKDTLM ISRTPEVTCVWVDVSHEDPEVKFNWYVDGVEVHNAKTKPPEEQYNSTLRVVSVLTVLHQDWLNG KEYKCKVSNWALPAPISKTISKAKGQPREPQVYTLPPSRDELTKNQVSLTCLVKGFYPSDIAVEW ESNGQPENNYKTTPLVLDSDGSFFLYSKLTVDKSRWQQGNVFSCSVMHEALHNHYTQKSLSLS PG (SEQ ID NO: 181); and
a light chain (LC) having the amino acid sequence DVVMTQTPLSLPVSLGDQASISCRCTQSLLHTNGDTYLHWYLQKPGQSPKLLIYKVSNRFSGVPD RFSGSGSGTDFTLKISRVEAEDLGVYFCSQSTHVPYTFGGGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 177); or
(18) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVTVPSSSLGTQTYICNVNHKPSNTKVDKKVEPKSCDKTHTCPPCPAPELLGGPSVFLF PPKPKDTLMISRTPEVTCVVVDVSHEDPEVKFNWYVDGVEVHNAKTKPREEQYNSTYRVVSVLT VLHQDWLNGKEYKCKVSNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 182); and
a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(19) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPAPEEKTISKAKGQPREPQVYTLPPSRDELTKNQVSLTCLVKG FYPSDIAVEWESNGQPENNYKTTPPVLDSDGSFFLYSKLTVDKSRWQQGNVFSCSVMHEALHN HYTQKSLSLSPG (SEQ ID NO: 184); and
a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASWVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(20) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPDVFLF PPKPKDTLMISRTPEVTCVVVDVSHEDPEVKFNWYVDGVEVHNAKTKPREEQYNSTYRVVSVLT VLHQDWLNGKEYKCKVSNKALPLPEEKTISKAKGQPREPQVYTLPPSRDELTKNQVSLTCLVKGF YPSDIAVEWESNGQPENNYKTTPPVLDSDGSFFLYSKLTVDKSRWQQGNVFSCSVMHEALHNH YTQKSLSLSPG (SEQ ID NO: 185); and
a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(21) a heavy chain (HC) having the amino acid sequence EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNKALPAPIEKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 186); and
a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASWVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(22) a heavy chain amino acid sequence (HC) having the EVOLVESGGGLVQPKGSLKLSCAASGFTFNTYAMNWVRQAPGKGLEWVARIRSKSNNYATYYA DSVKDRFTISRDDSQSMLYLQMNNLKTEDTAMYYCVRGREAYYRYDGGYYAMDYWGQGTSVT VSSASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELVGGPSVFLL PPKPKDTLMISRTPEVTCVVVDVSHEDPEVKFNWYVDGVEVHNAKTKPPEEQYNSTLRVVSVLT VLHQDWLNGKEYKCKVSNWALPAPISKTISKAKGQPREPQVYTLPPSRDELTKNQVSLTCLVKGF YPSDIAVEWESNGQPENNYKTTPLVLDSDGSFFLYSKLTVDKSRWQQGNVFSCSVMHEALHNH YTQKSLSLSPG (SEQ ID NO: 187); and
a light chain (LC) having the amino acid sequence DIVMTQAAPSVPVTPGESVSISCRSSKSLLHSNGNTYLYWFLQRPGQSPQLLIYRMSNLASGVPD RFSGSGSGTAFTLRISRVEAEDVGVYYCMQHLEYPFTFGSGTKLEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO: 183); or
(23) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPSVFLFPPKPKDTLMI SRTPEVTCVVVDVSHEDPEVKFNWYVDGVEVHNAKTKPREEQYNSTYRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPPVLDSDGSFFLYSKLTVDKSRWQQGNVFSCSVMHEALHNHYTQKSLSLSP G (SEQ ID NO: 188); and
a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
(24) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPDVFLFPPKPKDTLMI SRTPEVTCVVVDVSHEDPEVKFNWYVDGVEVHNAKTKPREEQYNSTYRVVSVLTVLHQDWLNG KEYKCKVSNKALPAPEEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 190); and
a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
(25) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPDVFLFPPKPKDTLMI SRTPEVTCVVVDVSHEDPEVKFNWYVDGVEVHNAKTKPREEQYNSTYRVVSVLTVLHQDWLNG KEYKCKVSNKALPLPEEKTISKAKGQPREPQVYTLPPSRDELTKNQVSLTCLVKGFYPSDIAVEW ESNGQPENNYKTTPPVLDSDGSFFLYSKLTVDKSRWQQGNVFSCSVMHEALHNHYTQKSLSLS PG (SEQ ID NO: 191); and
a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
(26) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELVGGPSVFLLPPKPKDTLMI SRTPEVTCVVVDVSHEDPEVKFNWYVDGVEVHNAKTKPPEEQYNSTLRVVSVLTVLHQDWLNG KEYKCKVSNKALPAPIEKTISKAKGQPREPQVYTLPPSRDELTKNQVSLTCLVKGFYPSDIAVEWE SNGQPENNYKTTPLVLDSDGSFFLYSKLTVDKSRWQQGNVFSCSVMHEALHNHYTQKSLSLSP G (SEQ ID NO: 192); and
a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WVCLLNNFYPREAKVQWKVDNALQSGNSQESVTEQDSKDSTYSLSSTLTLSKADYEKHKVYACE VTHQGLSSPVTKSFNRGEC (SEQ ID NO: 189); or
(27) a heavy chain (HC) having the amino acid sequence QVQLVETGGGLVRPGNSLKLSCVTSGFTFSNYRMHWLRQPPGKRLEWIAVIKVKSDNYGANYAE SVKGRFTISRDDSKSSVYLQVNRLREEDTATYYCSSPTYPGSSGFAYWGQGTLVTVSAASTKGP SVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELVGGPSVFLLPPKPKDTLMI SRTPEVTCVVVDVSHEDPEVKFNWYVDGVEVHNAKTKPPEEQYNSTLRVVSVLTVLHQDWLNG KEYKCKVSNWALPAPISKTISKAKGQPREPQVYTLPPSRDELTKNQVSLTCLVKGFYPSDIAVEW ESNGQPENNYKTTPLVLDSDGSFFLYSKLTVDKSRWQQGNVFSCSVMHEALHNHYTQKSLSLS PG (SEQ ID NO: 193); and
a light chain (LC) having the amino acid sequence DIQMTQTTSSLSASLGDRVTISCRASQDISNYLNWYQQKPDGTVKLLIYYTSRLHSGVPSRFSGS GSGTDYSLTISNLEQEDIATYFCQQGNKFPPTFGGGTKLEINRTVAAPSVFIFPPSDEQLKSGTAS WCLLNNFYPREAKVQWKVDNALQSGNSQESVTEQDSKDSTYSLSSTLTLSKADYEKHKVYACE VTHQGLSSPVTKSFNRGEC (SEQ ID NO: 189).

11. The antibody or antigen-binding fragment according to any one of claims 1 to 10, wherein the antibody or antigen-binding fragment comprises an Fc region, preferably an IgG1 Fc region.

12. The antibody or antigen-binding fragment according to any one of claims 1 to 11, wherein the antibody or antigen-binding fragment has ADCC, CDC and/or ADCP activity.

13. The antibody or antigen-binding fragment according to any one of claims 1 to 12, wherein the antibody or antigen-binding fragment comprises a hypofucosylated or an afucosylated Fc region.

14. The antibody or antigen-binding fragment according to any one of claims 1 to 13, wherein the antibody or antigen-binding fragment comprises an Fc region having one or more mutations enhancing ADCC and/or CDC and/or ADCP activity.

15. The antibody or antigen-binding fragment according to any one of claims 1 to 14, wherein the antibody or antigen-binding fragment has an activity of depleting CCR8-positive cells.

16. The antibody or antigen-binding fragment according to any one of claims 1 to 15, wherein the antibody or antigen-binding fragment has an activity of depleting CCR8-positive immune cells.

17. The antibody or antigen-binding fragment according to any one of claims 1 to 15, wherein the antibody or antigen-binding fragment has an activity of depleting CCR8-positive cancer cells.

18. The antibody or antigen-binding fragment according to any one of claims 1 to 17, wherein the antibody or antigen-binding fragment has an activity of inhibiting the CCL1-induced migration of CCR8-positive cells.

19. The antibody or antigen-binding fragment according to any one of claims 1 to 18, wherein the antibody or antigen-binding fragment has an activity of inhibiting the CCL1-induced activation of CCR8-positive cells.

20. The antibody or antigen-binding fragment according to any one of claims 1 to 19, wherein the antibody or antigen-binding fragment does not bind to peripheral immune cells from a healthy donor.

21. A nucleic acid encoding the heavy chain and/or the light chain of the antibody or antigen-binding fragment according to any one of claims 1 to 20.

22. A vector comprising the nucleic acid according to claim 21.

23. A host cell comprising the nucleic acid according to claim 21 or the vector according to claim 22.

24. A method of producing the antibody or antigen-binding fragment according to any one of claims 1 to 20, the method comprising culturing the host cell according to claim 23 and isolating the antibody or antigen-binding fragment.

25. The method of claim 24, wherein the host cell is a host cell for hypofucosylation or afucosylation.

26. An antibody or antigen-binding fragment as defined in any one of claims 1 to 20, which is obtainable by the method of claim 24 or 25.

27. An antibody or antigen-binding fragment as defined in any one of claims 1 to 20, which is obtainable by using a production cell line for hypofucosylation or afucosylation.

28. A composition comprising the antibody or antigen-binding fragment according to any one of claim 1 to 20, 26 or 27 or the nucleic acid according to claim 21, wherein said composition is preferably a pharmaceutical composition.

29. A lipid particle comprising one or more nucleic acids according to claim 21.

30. The nucleic acid according to claim 21 or the lipid particle according to claim 29, wherein the nucleic acid(s) is/are mRNA.

31. A chimeric antigen receptor (CAR) comprising an antibody or antigen-binding fragment as defined in any one of claim 1 to 6, 8 or 9, preferably comprising a single-chain variable fragment (scFv) according to any one of claim 1 to 6, 8 or 9.

32. An immune cell expressing the chimeric antigen receptor according to claim 31, wherein said immune cell is preferably a T cell or a natural killer cell, more preferably a T cell, even more preferably an alpha-beta-T cell or a gamma-delta-T cell.

33. The antibody or antigen-binding fragment according to any one of claim 1 to 20, 26 or 27, the nucleic acid according to claim 21 or 30, the composition according to claim 28, the lipid particle according to claim 29 or 30, or the immune cell according to claim 32, for use in the treatment of cancer.

34. The antibody or antigen-binding fragment for use according to claim 33, the nucleic acid for use according to claim 33, the composition for use according to claim 33, the lipid particle for use according to claim 33, or the immune cell for use according to claim 33, wherein said cancer is a solid cancer.

35. The antibody or antigen-binding fragment for use according to claim 33, the nucleic acid for use according to claim 33, the composition for use according to claim 33, the lipid particle for use according to claim 33, or the immune cell for use according to claim 33, wherein said cancer is a hematological cancer.

36. The antibody or antigen-binding fragment for use according to claim 33, the nucleic acid for use according to claim 33, the composition for use according to claim 33, the lipid particle for use according to claim 33, or the immune cell for use according to claim 33, wherein said cancer is selected from ovarian cancer, colorectal cancer, colon cancer, gastric cancer, esophageal cancer, breast cancer, lung cancer, bladder cancer, uterine cancer, urothelial cancer, Kaposi's sarcoma, skin cancer, head and/or neck cancer, renal cancer, and lymphoma.

37. The antibody or antigen-binding fragment for use according to any one of claims 33 to 36, the nucleic acid for use according to any one of claims 33 to 36, the composition for use according to any one of claims 33 to 36, the lipid particle for use according to any one of claims 33 to 36, or the immune cell for use according to any one of claims 33 to 36, wherein said treatment comprises the combined administration of one or more immune checkpoint inhibitors; preferably wherein said one or more immune checkpoint inhibitors are selected from anti-CTLA-4 antibodies, anti-PD-1 antibodies, anti-PD-L1 antibodies, anti-TIGIT antibodies, anti-TIM3 antibodies, anti-LAG3 antibodies, anti-OX40 antibodies, and anti-ICOS antibodies; more preferably wherein said one or more immune checkpoint inhibitors are selected from ipilimumab, tremelimumab, nivolumab, pembrolizumab, pidilizumab, cemiplimab, dostarlimab, spartalizumab, camrelizumab, sintilimab, tislelizumab, toripalimab, zimberelimab, AMP-224, AMP-514, JTX-4014, INCMGA00012, APE02058, atezolizumab, avelumab, durvalumab, KN035, CK-301, BMS-936559, MEDI4736, MPDL3280A, MDX-1105, MEDI6469, bintrafusp alfa, tiragolumab, vibostolimab, domvanalimab, etigilimab, BMS-986207, EOS-448, COM902, ASP8374, SEA-TGT, BGB-A1217, IBI939, M6223, cobolimab, sabatolimab, BMS-986258, Sym023, TQB2618, LY3321367, SHR-1702, relatlimab, ieramilimab, encelimab, tebotelimab, REGN3767, FS118, IMP701, IMP731, ivuxolimab, MEDI0562, MEDI6383, MEDI6469, INCAGNO1949, ABBV-368, BAT6026, BGB-A445, YH-002, BMS 9861708, INBRX-106, IBI101, MOXR0916, alomfilimab, feladilimab, vopratelimab, BMS-986226, MEDI-570, and XmAb23104.
